(19) United States
(12) Patent Application Publication
(10) Pub. No.: US 2025/0326866 A1
(43) Pub. Date: Oct. 23, 2025

(54) TRANSFERRIN RECEPTOR TARGETING PEPTIDE OLIGONUCLEOTIDE COMPLEXES AND METHODS OF USE THEREOF

(71) Applicant: Blaze Bioscience, Inc., Seattle, WA (US)

(72) Inventors: Zachary CROOK, Seattle, WA (US); Natalie Winblade NAIRN, Seattle, WA (US); Julia E. NOVAK, Sequim, WA (US)

(21) Appl. No.: 18/683,490

(22) PCT Filed: Aug. 16, 2022
(86) PCT No.: PCT/US2022/040432

§ 371 Date: Feb. 13, 2024

Related U.S. Application Data
(60) Provisional application No. 63/234,150, filed on Aug. 17, 2021.

(51) Int. Cl. C07K 19/00; A61K 47/54; A61K 47/64; A61K 49/00; A61K 51/08

(52) U.S. Cl. CPC C07K 19/00; A61K 47/549; A61K 47/6455; A61K 49/0002; A61K 51/088; A61K 2121/00

(57) ABSTRACT

Described herein are peptide oligonucleotide complexes comprising peptides and variants thereof as well as conjugated to, linked to, or fused to agents, wherein the peptide oligonucleotide complexes are capable of binding TfR and that are capable of modulating RNA or proteins in or on cells. Binding of a peptide oligonucleotide complex as described herein to TfR can enable transcytosis of complexes across an endothelial layer, e.g., the blood brain barrier, or the crossing of a cell membrane, such as by endocytosis. Pharmaceutical compositions and uses of such peptide oligonucleotide complexes, as well as methods of designing and manufacturing such peptide oligonucleotide complexes, to treat a disease or condition are also described herein.

### CROSS-REFERENCE

The present application claims the benefit of U.S. Provisional Application No. 63/234,150, entitled “TRANSFERRIN RECEPTOR TARGETING PEPTIDE OLIGONUCLEOTIDE COMPLEXES AND METHODS OF USE THEREOF,” filed on Aug. 17, 2021, which application is herein incorporated by reference in its entirety for all purposes.

### SEQUENCE LISTING

The instant application contains a Sequence Listing which has been submitted electronically in eXtensible Markup Language (XML file) format and is hereby incorporated by reference in its entirety. Said XML copy, created on Sep. 1, 2022, is named 438542-722021_SL.xml and is 705,319 bytes in size.

### BACKGROUND

Drug delivery of oligonucleotides to their intracellular targets is hampered by the ability to direct the oligonucleotide to the appropriate tissue or cell. The blood brain barrier (BBB) exists to keep toxic metabolites and pathogens out of the brain, but also serves to render diseases of the CNS particularly difficult to treat using conventional medicines. Thus, approaches for an improved delivery of therapeutic and/or diagnostic oligonucleotides into the CNS and other organs and tissues for indications including inflammation, neurodegeneration, oncology, infectious disease, cardiovascular and other areas are needed.

### SUMMARY

The present disclosure relates to compositions and methods for treatment of disorders. Described herein are peptide oligonucleotide complexes comprising nucleic acids as targeted agents against disease, such nucleic acids including a nucleotide antisense RNA, a complementary RNA, an inhibitory RNA, an interfering RNA, a nuclear RNA, an antisense oligonucleotide (ASO), a microRNA (miRNA), an oligonucleotide complementary to a natural antisense transcripts (NATs) sequence, an siRNA, an snRNA, an aptamer, a gapmer, an anti-miR, a splice blocker ASO, or a U1 adapter that selectively homes, targets, is directed to, migrates to, is able to reach, is directed into the lysosomal pathway or other subcellular compartment, is retained by, or accumulates in and/or binds to specific regions, tissues, structures or cells of the central nervous system (CNS), muscle, spleen, bone marrow, GI tract, liver, and many tumors, or that are involved in sensing, modulating, managing, decreasing, ablating or reducing pain, including nociceptive pain, and are useful for delivery of therapeutic and/or diagnostic oligonucleotides into the CNS and other organs and tissues for indications in inflammation, neurodegeneration, oncology, infectious disease, cardiovascular and other therapeutic indications as described herein following administration in a subject. In some embodiments, the peptide complexes of the present disclosure are used to deliver a detection agent to image and/or diagnose cartilage, injury, or disease. In some embodiments, compositions and methods for treatment of using the peptide complexes are described. In other embodiments, the peptide complexes of the present disclosure are used to treat or deliver an active agent to a region, tissue, structure, or cell thereof.

In various aspects, the present disclosure provides a peptide oligonucleotide complex comprising a peptide and an oligonucleotide, wherein the peptide comprises a transferrin receptor-binding peptide capable of binding a transferrin receptor, and wherein the oligonucleotide comprises a target-binding agent capable of binding a target molecule.

In some aspects, the oligonucleotide binds to the target molecule with a melting temperature of not less than 37° C. and not more than 99° C. In some aspects, the oligonucleotide binds to the target molecule with a melting temperature of not less than 40° C. and not more than 85° C., not less than 40° C. and not more than 65° C., not less than 40° C. and not more than 55° C., not less than 50° C. and not more than 85° C., not less than 60° C. and not more than 85° C., or not less than 55° C. and not more than 65° C. In some aspects, the oligonucleotide binds the target molecule with an affinity of: not more than 500 nM, not more than 100 nM, not more than 50 nM, not more than 10 nM, not more than 1 nM, not more than 500 μM, not more than 400 μM, not more than 300 μM, not more than 200 μM, or not more than 100 μM; or not more than 500 nM and not less than 100 μM, not more than 100 nM and not less than 200 μM, not more than 50 nM and not less than 300 μM, not more than 10 nM and not less than 400 μM, or not more than 1 nM and not less than 500 μM.

In some aspects, the oligonucleotide comprises a G/C content of not more than 80%, not more than 75%, not more than 70%, not more than 65%, or not more than 50%. In some aspects, the oligonucleotide comprises a G/C content of not less than 20%, not less than 25%, not less than 30%, not less than 35%, not less than 40%, not less than 45%, or not less than 50%. In some aspects, the oligonucleotide comprises a G/C content of not less than 20% and not more than 80%, not less than 30% and not more than 65%, or not less than 40% and not more than 55%. In some aspects, the oligonucleotide comprises: an A/T content of not less than 20%, not less than 25%, not less than 30%, not less than 35%, not less than 40%, not less than 45%, or not less than 50%; an A/U content of not less than 20%, not less than 25%, not less than 30%, not less than 35%, not less than 40%, not less than 45%, or not less than 50%; or a combination thereof. In some aspects, the oligonucleotide comprises: an A/T content of not more than 80%, not more than 75%, not more than 70%, not more than 65%, or not more than 50%; an A/U content of not more than 80%, not more than 75%, not more than 70%, not more than 65%, or not more than 50%; or a combination thereof. In some aspects, the oligonucleotide comprises: an A/T content of not less than 20% and not more than 80%, not less than 30% and not more than 65%, or not less than 40% and not more than 55%; an A/U content of not less than 20% and not more than 80%, not less than 30% and not more than 65%, or not less than 40% and not more than 55%; or a combination thereof.

In some aspects, a single strand of the oligonucleotide has a length of not more than 500 nt, not more than 300 nt, not more than 100 nt, not more than 50 nt, not more than 30 nt, not more than 28 nt, not more than 26 nt, not more than 25 nt, not more than 24 nt, not more than 23 nt, not more than 22 nt, not more than 21 nt, 20 nt, not more than 19 nt, not more than 18, nt, not more than 17 nt, not more than 16 nt, not more than 15 nt, or not more than 12 nt. In some aspects, a single strand of the oligonucleotide has a length of not less than 12 and not more than 50 nt, not less than 12 and not more than 30 nt, not less than 12 and not more than 25 nt, not less than 18 and not more than 25 nt, not less than 18 and not more than 24 nt, not less than 19 and not more than 23 nt, or not less than 20 and not more than 22 nt. In some aspects, a single strand of the oligonucleotide has a length of 21 nt±2 nt.

In some aspects, the oligonucleotide comprises a nucleotide antisense RNA, a complementary RNA, an inhibitory RNA, an interfering RNA, a nuclear RNA, an antisense oligonucleotide, a microRNA, a sequence complementary to a natural antisense transcript, a small interfering RNA, a small nuclear RNA, an aptamer, a gapmer, an anti-miR sequence, a splice blocker antisense oligonucleotide, or a U1 adapter. In some aspects, the oligonucleotide comprises a small nuclear RNA, an aptamer, a gapmer, an anti-miR sequence, a splice blocker antisense oligonucleotide, or a U1 adapter. In some aspects, the oligonucleotide comprises at least 90%, at least 91%, at least 92%, at least 93%, at least 94%, at least 95%, at least 96%, at least 97%, at least 98%, or at least 99% sequence identity to any one of SEQ ID NO: 364-SEQ ID NO: 394. In some aspects, the oligonucleotide comprises a sequence of any one of SEQ ID NO: 364-SEQ ID NO: 394, any one of SEQ ID NO: 364-SEQ ID NO: 394 wherein U is replaced with T, or any one of SEQ ID NO: 364-SEQ ID NO: 394 wherein T is replaced with U. In some aspects, the oligonucleotide comprises no more than 1, 2, 3, 4, or 5 base changes relative to a sequence of any one of SEQ ID NO: 364-SEQ ID NO: 394. In some aspects, the oligonucleotide comprises a single stranded oligonucleotide. In some aspects, the oligonucleotide comprises a double stranded oligonucleotide.

In some aspects, the oligonucleotide comprises at least one phosphorothioate linkage. In some aspects, the peptide oligonucleotide complex comprises from 1 to 12 phosphorothioate linkages. In some aspects, the oligonucleotide comprises at least one thiophosphoroamidate linkage. In some aspects, the peptide oligonucleotide complex comprises from 1 to 12 thiophosphoroamidate linkages. In some aspects, the peptide oligonucleotide complex comprises a phosphodiester linkage, a phosphorodiamidate linkage, or a combination thereof. In some aspects, the peptide oligonucleotide complex comprises from 1 to 12 phosphodiester linkages, from 1 to 12 phosphorodiamidate linkages, or a combination thereof. In some aspects, the oligonucleotide comprises at least one modified base. In some aspects, the at least modified base comprises a 2′F base, an LNA base, a BNA base, an ENA base, a 2′O-MOE base, a 5′-Me base, a (S)-cEt base, a 2′OMe base, a morpholino base, or combinations thereof.

In some aspects, the oligonucleotide binds to a U1 snRNA. In some aspects, the oligonucleotide comprises a U1 snRNA-binding sequence that is reverse complementary to a U1 snRNA. In some aspects, the U1 snRNA-binding sequence has a length of at least 13 nt. In some aspects, the oligonucleotide comprises a U1 adapter. In some aspects, the U1 adaptor comprises a sequence of any one of SEQ ID NO: 364-SEQ ID NO: 371. In some aspects, the oligonucleotide comprises an anti-miR sequence. In some aspects, the anti-miR sequence comprises a sequence of any one of SEQ ID NO: 372-SEQ ID NO: 379. In some aspects, the oligonucleotide comprises a small interfering RNA. In some aspects, the small interfering RNA comprises a sequence of any one of SEQ ID NO: 387-SEQ ID NO: 394. In some aspects, the oligonucleotide comprises a gapmer. In some aspects, the gapmer comprises a sequence of any one of SEQ ID NO: 381-SEQ ID NO: 386. In some aspects, the oligonucleotide comprises an aptamer. In some aspects, the target molecule is a target protein, and wherein the aptamer binds the target protein.

In some aspects, the target molecule comprises a gene, an open reading frame, an mRNA, a pre-mRNA, or a protein. In some aspects, the target molecule comprises a molecule listed in TABLE 4, TABLE 5, or TABLE 6, a DNA sequence encoding a molecule listed in TABLE 4, TABLE 5, or TABLE 6, an RNA sequence encoding a molecule listed in TABLE 4, TABLE 5, or TABLE 6. In some aspects, the target molecule comprises a sequence of any one of SEQ ID NO: 395-SEQ ID NO: 428 provided in TABLE 3 or an open reading frame listed in TABLE 18, or a fragment thereof. In some aspects, the target molecule encodes a pro-inflammatory cytokine, an extracellular matrix-modifying protein, TNF-α, ICAM-1, a p65 subunit of NF-κB, Smad7, carbohydrate sulfotransferase 15, IL-23, IL-12, IL-17, poly-Q expanded huntingtin, amyloid precursor protein, microtubule associated protein tau, SMN2, SCN1A, ASO, SCN8A, IGF-1, IGF-1 receptor, EGFR, ERBB3, HER2, GRB2, KRAS, MYC, YAP1, a heat shock protein, a hypoxia-sensing protein, MDM2, BCL2, FOXP3, DNMT1, an HDAC, a parasite surface protein, GPX₄, SLC7a11, α-synuclein, a JAK-STAT pathway protein, a viral protein, or LRRK2.

In some aspects, the oligonucleotide is at least 60%, at least 70%, at least 80%, at least 90%, at least 92%, at least 93%, at least 94%, at least 95%, at least 96%, at least 97%, at least 98%, at least 99%, or 100% reverse complementary to the target molecule. In some aspects, the oligonucleotide is 100% reverse complementary to the target molecule. In some aspects, the oligonucleotide is at least 60%, at least 70%, at least 80%, at least 90%, at least 91%, at least 92%, at least 93%, at least 94%, at least 95%, at least 96%, at least 97%, at least 98%, at least 99%, or 100% reverse complementary to a portion of a DNA sequence or RNA sequence encoding a molecule listed in TABLE 4, TABLE 5, or TABLE 6. In some aspects, the oligonucleotide is 100% reverse complementary to a portion of a DNA sequence or RNA sequence encoding a molecule listed in TABLE 4, TABLE 5, or TABLE 6. In some aspects, the oligonucleotide is at least 60%, at least 70%, at least 80%, at least 90%, at least 91%, at least 92%, at least 93%, at least 94%, at least 95%, at least 96%, at least 97%, at least 98%, at least 99%, or 100% reverse complementary to a sequence of any one of SEQ ID NO: 395-SEQ ID NO: 428 provided in TABLE 3 or an open reading frame listed in TABLE 18, or a fragment thereof. In some aspects, the oligonucleotide is 100% reverse complementary to a sequence of any one of SEQ ID NO: 395-SEQ ID NO: 428 provided in TABLE 3 or an open reading frame listed in TABLE 18, or a fragment thereof. In some aspects, the oligonucleotide comprises no more than 1, 2, 3, 4, or 5 base pair mismatches upon binding to the target molecule. In some aspects, the oligonucleotide comprises at least 1, 2, 3, 4, or 5 base pair mismatches upon binding to the target molecule. In some aspects, the oligonucleotide comprises a sequence reverse complementary to a sequence encoding a pro-inflammatory cytokine, an extracellular matrix-modifying protein, TNF-α, ICAM-1, a p65 subunit of NF-κB, Smad7, carbohydrate sulfotransferase 15, IL-23, IL-12, IL-17, poly-Q expanded huntingtin, amyloid precursor protein, microtubule associated protein tau, SMN2, SCN1A, ASO, SCN8A, IGF-1, IGF-1 receptor, EGFR, ERBB3, HER2, GRB2, KRAS, MYC, YAP1, a heat shock protein, a hypoxia-sensing protein, MDM2, BCL2, FOXP3, DNMT1, an HDAC, a parasite surface protein, GPX₄, SLC7a11, α-synuclein, a JAK-STAT pathway protein, a viral protein, or LRRK2, or a fragment thereof.

In some aspects, the target molecule is a pro-inflammatory cytokine, an extracellular matrix-modifying protein, TNF-α, ICAM-1, a p65 subunit of NF-κB, Smad7, carbohydrate sulfotransferase 15, IL-23, IL-12, IL-17, poly-Q expanded huntingtin, amyloid precursor protein, microtubule associated protein tau, SMN2, SCN1A, ASO, SCN8A, IGF-1, IGF-1 receptor, EGFR, ERBB3, HER2, GRB2, KRAS, MYC, YAP1, a heat shock protein, a hypoxia-sensing protein, MDM2, BCL2, FOXP3, DNMT1, an HDAC, a parasite surface protein, GPX₄, SLC7a11, α-synuclein, a JAK-STAT pathway protein, a viral protein, or LRRK2. In some aspects, the oligonucleotide binds a pro-inflammatory cytokine, an extracellular matrix-modifying protein, TNF-α, ICAM-1, a p65 subunit of NF-κB, Smad7, carbohydrate sulfotransferase 15, IL-23, IL-12, IL-17, poly-Q expanded huntingtin, amyloid precursor protein, microtubule associated protein tau, SMN2, SCN1A, ASO, SCN8A, IGF-1, IGF-1 receptor, EGFR, ERBB3, HER2, GRB2, KRAS, MYC, YAP1, a heat shock protein, a hypoxia-sensing protein, MDM2, BCL2, FOXP3, DNMT1, an HDAC, a parasite surface protein, GPX₄, SLC7a11, α-synuclein, a JAK-STAT pathway protein, a viral protein, or LRRK2.

In some aspects, the peptide binds the transferrin receptor with an affinity of no more than 10 nM, 5 nM, 1 nM, 800 μM, 600 μM, 500 μM, 400 μM, 300 μM, 250 μM, or 200 μM. In some aspects, the affinity is lower at pH 7.0 than at pH 7.4, lower at pH 6.5 than at pH 7.4, lower at pH 6.0 than at pH 7.4, or lower at pH 5.5 than at pH 7.4, or lower at pH 5.0 than at pH 7.4. In some aspects, the affinity is higher at pH 7.5 than at pH 5.5. In some aspects, the affinity at pH 7.5 is at least 0.25-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, at least 100-fold, at least 500-fold, at least 1000-fold, at least 5000-fold, or at least 10,000-fold higher than the affinity at pH 5.5.

In some aspects, the peptide comprises a sequence having at least 80%, at least 90%, at least 91%, at least 92%, at least 93%, at least 94%, at least 95%, at least 96%, at least 97%, at least 98%, or at least 99% sequence identity to any one of SEQ ID NO: 1-SEQ ID NO: 134, a fragment thereof, a variant thereof, a homolog thereof, or an analog thereof. In some aspects, the peptide comprises a sequence of any one of or SEQ ID NO: 306-SEQ ID NO: 335. In some aspects, the peptide comprises a sequence of any one of SEQ ID NO: 1-SEQ ID NO: 134, a fragment thereof, a variant thereof, a homolog thereof, or an analog thereof. In some aspects, the peptide comprises a sequence of any one of SEQ ID NO: 1-SEQ ID NO: 134. In some aspects, the peptide comprises a sequence of SEQ ID NO: 32. In some aspects, the peptide comprises a sequence of SEQ ID NO: 2. In some aspects, the peptide comprises a sequence of SEQ ID NO: 64. In some aspects, the peptide comprises a sequence of SEQ ID NO: 34. In some aspects, the peptide comprises a sequence of any one of SEQ ID NO: 129-SEQ ID NO: 134.

In some aspects, the peptide comprises at least one disulfide bond, at least two disulfide bonds, at least three disulfide bonds, at least four disulfide bonds, or at least five disulfide bonds. In some aspects, the peptide comprises at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 amino acid residues. In some aspects, the peptide comprises not more than 49, not more than 50, not more than 51, not more than 52, not more than 53, not more than 54, not more than 55, not more than 60, not more than 65, not more than 70, not more than 75, not more than 80, not more than 85, not more than 90, not more than 95, or not more than 100 amino acid residues.

In some aspects, the oligonucleotide is linked to the peptide via a linker. In some aspects, the linker is a stable linker. In some aspects, the linker is a cleavable linker. In some aspects, the cleavable linker is cleaved in an endosome. In some aspects, the linker is selected from TABLE 10, TABLE 11, any one of SEQ ID NO: 234-SEQ ID NO: 297, or combinations thereof. In some aspects, the linker comprises a triazole linker, a linear linker, a non-cyclic linker, a cyclic linker, a cyclic carboxylic acid linker, ester linkage, linear dicarboxylic acid linker, an amino acid linker, or a combination thereof. In some aspects, the linker comprises a triazole linker. In some aspects, the triazole linker comprises a 1,2,3-triazole or a 1,2,4-triazole.

In some aspects, the peptide oligonucleotide complex further comprising an additional cell penetrating moiety. In some aspects, the additional cell penetrating moiety is fused to or conjugated to the peptide. In some aspects, the additional cell penetrating moiety is conjugated to the oligonucleotide. In some aspects, the additional cell penetrating moiety comprises a polycation, a polyorganic acid, an endosomal releasing polymer, poly(2-propylacrylic acid), poly(2-ethylacrylic acid), a Tat peptide, an Arg patch, a knotted peptide, CysTAT, S19-TAT, R8 (SEQ ID NO: 143), pAntp, Pas-TAT, Pas-R8 (SEQ ID NO: 146), Pas-FHV, Pas-pAntP, F2R4 (SEQ ID NO: 149), B55, aurein, IMT-P8, BR2, OMOTAG1, OMOTAG2, pVEC, SynB3, DPV1047, C105Y, Transportan, MTS, hLF, PFVYLI (SEQ ID NO: 163), maurocalcine, imperatoxin, hadrucalin, hemicalcin, opicalcin-1, opicalcin-2, midkine (62-104), MCoTI-II, chlorotoxin, DRI-TAT, cFΦDR4 (SEQ ID NO: 166), R6W3 (SEQ ID NO: 189), myristate, yBBR, or a fragment or variant thereof, or any combination thereof. In some aspects, the additional cell penetrating moiety comprises a sequence of any one of SEQ ID NO: 141-SEQ ID NO: 233.

In some aspects, the peptide oligonucleotide complex further comprises an active agent. In some aspects, the active agent is conjugated to, linked to, or fused to the peptide. In some aspects, the active agent is conjugated to or linked to the oligonucleotide. In some aspects, the active agent comprises a radionuclide, a radionuclide chelator, a chelator, an immunotherapeutic agent, a CTLA-4 targeting agent, a PD-1 targeting agent, a PDL-1 targeting agent, an IL15 agent, a fused IL-15/IL-15Ra complex agent, an IFNgamma agent, an anti-CD3 agent, an ion channel modulator, a Kv1.3 inhibitor, an auristatin, MMAE, a maytansinoid, DM1, DM4, doxorubicin, a calicheamicin, a platinum compound, cisplatin, a taxane, paclitaxel, SN-38, a BACE inhibitor, a Bcl-xL inhibitor, WEHI-539, venetoclax, ABT-199, navitoclax, AT-101, obatoclax, a pyrrolobenzodiazepine or pyrrolobenzodiazepine dimer, a dolastatin, or a neurotransmitter.

In some aspects, the peptide oligonucleotide further comprises a detectable agent. In some aspects, the detectable agent is a fluorophore, a near-infrared dye, a contrast agent, a nanoparticle, a metal-containing nanoparticle, a metal chelate, an X-ray contrast agent, a PET agent, a radionuclide, or a radionuclide chelator. In some aspects, the detectable agent is linked to the peptide or the oligonucleotide via a linker. In some aspects, the active agent is linked to the peptide or the oligonucleotide via a linker. In some aspects, the linker is a stable linker. In some aspects, the linker is a cleavable linker. In some aspects, the cleavable linker is cleaved in an endosome. In some aspects, the linker is selected from TABLE 10, TABLE 11, any one of SEQ ID NO: 234-SEQ ID NO: 297, or combinations thereof. In some aspects, the linker comprises a triazole linker, a linear linker, a non-cyclic linker, a cyclic linker, a cyclic carboxylic acid linker, ester linkage, linear dicarboxylic acid linker, an amino acid linker, or a combination thereof. In some aspects, the linker comprises a triazole linker. In some aspects, the triazole linker comprises a 1,2,3-triazole or a 1,2,4-triazole.

In some aspects, the peptide oligonucleotide complex further comprises a half-life modifying agent coupled to the peptide or the oligonucleotide. In some aspects, the half-life modifying agent comprises a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, an SA21, or a molecule that binds to albumin. In some aspects, the SA21 comprises a sequence of SEQ ID NO: 357.

In some aspects, the peptide oligonucleotide complex is stable in human serum. In some aspects, at least 50% of the peptide oligonucleotide complex remains intact after incubation in human serum at 37° C. for up to 5 min, 15 min, 30 min, 45 min, 1 hour, 2 hours, 4 hours, 8 hours, 12 hours, or 24 hours.

In various aspects, the present disclosure provides a method of modulating an activity of a target molecule, the method comprising: contacting a cell with a peptide oligonucleotide complex comprising a peptide and an oligonucleotide, binding the peptide to a transferrin receptor; transporting the peptide oligonucleotide complex across a cellular layer of the cell; and binding the oligonucleotide to a target molecule, thereby modulating the activity of the target molecule.

In some aspects, the peptide oligonucleotide complex comprises any peptide oligonucleotide complex described herein. In some aspects, the peptide binds the transferrin receptor with an affinity of no more than 10 nM, 5 nM, 1 nM, 800 μM, 600 μM, 500 μM, 400 μM, 300 μM, 250 μM, or 200 μM.

In some aspects, modulating the activity of the target molecule comprises reducing expression of the target molecule, increasing the expression of the target molecule, reducing translation of the target molecule, degrading the target molecule, reducing a level of the target molecule, modifying the processing of the target molecule, modifying the splicing of the target molecule, inhibiting processing of the target molecule, reducing a level of a protein encoded by the target molecule, blocking an interaction with the target molecule, or combinations thereof. In some aspects, the expression of the target molecule is reduced by at least 10%, 25%, 30%, 40%, 50%, 60%, 75%, 80%, 90%, 95%, 99%, 99.5%, or 99.9%. In some aspects, the translation of the target molecule is reduced by at least 10%, 25%, 30%, 40%, 50%, 60%, 75%, 80%, 90%, 95%, 99%, 99.5%, or 99.9%. In some aspects, the expression of the target molecule is reduced by a factor of at least 2, 4, 8, 10, 15, 16, 20, 32, 50, 64, 100, 128, 200, 256, 500, 512, or 1000. In some aspects, translation of the target molecule is reduced by a factor of at least 2, 4, 8, 10, 15, 16, 20, 32, 50, 64, 100, 128, 200, 256, 500, 512, or 1000. In some aspects, at least 10%, 25%, 30%, 40%, 50%, 60%, 75%, 80%, 90%, 95%, 99%, 99.5%, or 99.9% of the target molecule is degraded. In some aspects, the level of the protein encoded by the target molecule is reduced by at least 10%, 25%, 30%, 40%, 50%, 60%, 75%, 80%, 90%, 95%, 99%, 99.5%, or 99.9%. In some aspects, modifying the splicing of the target molecule increases a level of a protein encoded by the target molecule by at least 10%, 25%, 30%, 40%, 50%, 60%, 75%, 80%, 90%, 95%, 99%, 99.5%, or 99.9%.

In some aspects, the protein encoded by the target molecule comprises a modification. In some aspects, the cellular layer is a plasma membrane, a blood brain barrier, a lysosomal membrane, an endosomal membrane, or a nuclear membrane. In some aspects, the transporting comprises transferrin receptor-mediated endocytosis or receptor-mediated transcytosis. In some aspects, the cell expresses the target molecule. In some aspects, the cell expresses the transferrin receptor. In some aspects, the cell is a cancer cell, a neuronal cell, a hematopoietic cell, a muscle cell, a lymphoid cell, or a gastrointestinal cell. In some aspects, the method further comprises releasing the peptide oligonucleotide complex from the transferrin receptor. In some aspects, at least 50% of the peptide oligonucleotide complex remains intact up to 5 min, 15 min, 30 min, 45 min, 1 hour, 2 hours, 4 hours, 8 hours, 12 hours, or 24 hours after the contacting.

In various aspects, the present disclosure provides a method of treating a condition in a subject in need thereof, the method comprising: administering to the subject a composition comprising a peptide oligonucleotide complex comprising a peptide and a nucleotide; binding the peptide to a transferrin receptor; delivering the peptide oligonucleotide complex across a cellular layer of the subject; binding the nucleotide to a target molecule; and modulating an activity of the target molecule associated with the condition, thereby treating the condition in the subject.

In some aspects, the peptide oligonucleotide complex comprises any peptide oligonucleotide complex described herein. In some aspects, the peptide binds the transferrin receptor with an affinity of no more than 10 nM, 5 nM, 1 nM, 800 μM, 600 μM, 500 μM, 400 μM, 300 μM, 250 μM, or 200 μM.

In some aspects, the condition is a neuronal condition, a gastrointestinal condition, an inflammatory condition, an immune condition, a neurological condition, a muscular condition, an infectious condition, or a cancer. In some aspects, the cancer is ovarian cancer, colon cancer, lung cancer, cancer located in the bone or bone marrow, glioblastoma, astrocytoma, glioma, medulloblastoma, ependymoma, choroid plexus carcinoma, midline glioma, diffuse intrinsic pontine glioma (DIPG), breast cancer, liver cancer, colon cancer, brain cancer, spleen cancer, cancers of the salivary gland, kidney cancer, muscle cancers, bone marrow cell cancers, skin cancer, genitourinary cancer, osteosarcoma, muscle-derived sarcoma, melanoma, head and neck cancer, neuroblastoma, prostate cancer, bladder cancer, acute lymphocytic leukemia, acute myeloid leukemia, chronic lymphocytic leukemia, chronic myeloid leukemia, Hodgkin lymphoma, Non-Hodgkin lymphoma, or a CMYC-overexpressing cancer. In some aspects, the gastrointestinal condition is inflammatory bowel disease, ulcerative colitis, or Crohn's disease. In some aspects, the neuronal condition is a neurodegenerative condition. In some aspects, the neurodegenerative condition is Alzheimer's disease, Parkinson's disease, Huntington's disease, amyotrophic lateral sclerosis, or frontotemporal dementia. In some aspects, the condition is selected from TABLE 4, TABLE 5, or TABLE 6.

In some aspects, treating the condition comprises reducing a phenotype associated with the condition in the subject. In some aspects, the phenotype is reduced by at least 10%, 25%, 30%, 40%, 50%, 60%, 75%, 80%, 90%, 95%, 99%, 99.5%, or 99.9%. In some aspects, the phenotype is tumor growth rate or neurodegenerative disease progression. In some aspects, treating the condition comprises reducing a symptom associated with the condition in the subject. In some aspects, the symptom is reduced by at least 10%, 25%, 30%, 40%, 50%, 60%, 75%, 80%, 90%, 95%, 99%, 99.5%, or 99.9%. In some aspects, the symptom is tumor growth rate or neurodegenerative disease progression.

In some aspects, the cellular layer is a plasma membrane, a blood brain barrier, a lysosomal membrane, an endosomal membrane, or a nuclear membrane. In some aspects, the transporting comprises transferrin receptor-mediated endocytosis or receptor-mediated transcytosis. In some aspects, the peptide oligonucleotide complex is administered to the subject intranasally, orally, topically, intravenously, subcutaneously, intramuscularly administration, intraperitoneally, intratumorally, intrathecally, intravitreally, via inhalation, via suppository, or a combination thereof. In some aspects, the peptide oligonucleotide complex is administered intravenously as a bolus, infusion, or prolonged infusion.

In some aspects, the method further comprises releasing the peptide oligonucleotide complex from the transferrin receptor. In some aspects, the subject is a human or a non-human animal. In some aspects, at least 50% of the peptide oligonucleotide complex remains intact up to 5 min, 15 min, 30 min, 45 min, 1 hour, 2 hours, 4 hours, 8 hours, 12 hours, or 24 hours after the administering. In some aspects, at least 5%, at least 10%, at least 20%, at least 30%, at least 40%, at least 50%, at least 60%, at least 70%, at least 80%, or at least 90% of the oligonucleotide remains intact after the administering.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates oligonucleotide mechanisms of action. Oligonucleotides, which are targeted to a specific sequence for its regulation, complexed with a TfR-binding CDP enter into cells through TfR-binding and natural endocytosis of TfR, resulting in oligonucleotide compartmentalization into endosomes. Oligonucleotides are released from the endocytic compartments into the cytoplasm where they can freely move between the nucleus and the cytoplasm. Upon entry into the nucleus, oligonucleotides can (1) modulate alternative splicing of a targeted sequence, (2) dictate the location of the polyadenylation (polyA) site of a targeted sequence, and (3) recruit RNaseH1 to induce cleavage of a targeted sequence. Oligonucleotides in the cytoplasm can be designed to (4) directly bind to microRNA (miRNA) or messenger (mRNA) sequences. siRNAs, which are targeted to a specific sequence for its regulation, may alternatively be used to (5) bind and regulate a targeted sequence in the cytosol, engaging an RNA-induced silencing complex (RISC) which is a multiprotein complex that incorporates one strand of a small interfering RNA (siRNA) or micro RNA (miRNA), using the siRNA or miRNA as a template for recognizing complementary mRNA of the targeted sequence. When it finds a complementary strand, the RISC complex cleaves the targeted sequence. An aptamer, targeted to a specific sequence for its regulation, may alternatively be used to (6) bind and regulate a target molecule. An aptamer directly binds and inhibits its intracellular or extracellular target.

FIG. 2 illustrates examples of structures of various peptide oligonucleotide complexes (e.g., a CDP-oligonucleotide complexes in which the peptide portion comprises a CDP) containing alternative and nonconventional bases, as represented in single-stranded, double-stranded, and hairpin structures. Examples of oligonucleotides include an aptamer, a gapmer, an anti-miR, an siRNA, a splice blocker ASO, and a U1 adapter. The CDP portion of the CDP-oligonucleotide complex can be used to guide the oligonucleotide sequence to a specific tissue, target, or cell. The legend is as follows: grey circle with black border=2′-H (DNA); white circle with black border=2′-OH (RNA); circle with horizontal stripes and black border=2′-O-ME; circle with vertical stripes=2′-O-MOE; black circle with grey border=2′-F; spotted circle with grey border=LNA; hatched circle with grey border=morpholino (unique phosphorodiamidate linkages not shown); grey angle=PO linkage; black angle=PS linkage.

FIG. 3 illustrates incorporation of the shown groups on RNA or DNA.

FIG. 3A illustrates structures of oligonucleotides containing a 5′-thiol (thiohexyl; C6) modification (left), and a 3′-thiol (C3) modification (right).

FIG. 3B illustrates an MMT-hexylaminolinker phosphoramidite.

FIG. 3C illustrates a TFA-pentylaminolinker phosphoramidite.

FIG. 3D illustrates RNA residues incorporating amine or thiol residues.

FIG. 3E illustrates oligonucleotides with aminohexyl modifications at the 5′ (left) and 3′ ends (right).

FIG. 4 illustrates that TfR-binding peptides are cross-reactive with murine TfR (mTfR) in cell surface binding assays. 293F cells expressing either human or mouse TfR from their surface were stained with soluble TfR-binding peptides that were directly labeled with AlexaFluor 647 dye.

FIG. 4A illustrates the species specificity of the TfR used in these experiments, in this case human TfR. Data is displayed as two topographical density maps and indicates flow cytometry data of transferrin stained with Anti-hTfR (CD71) antibody. The upper density map, oriented diagonally from lower left to upper right, depicts 293ST+SDGF-hTfR. The lower density map, oriented horizontally, depicts 293ST+SDGF-mTfR. The y-axis shows hTfR+Streptavidin from 0 to 10⁷, in increments of 10 on a log scale. The x-axis shows GFP from 0 to 10⁶, in increments of 10 on a log scale.

FIG. 4B illustrates the species specificity of the TfR used in these experiments, in this case murine TfR. Data is displayed as two topographical density maps and indicates flow cytometry data of transferrin stained with Anti-mTfR (CD71) antibody. The upper density map, oriented diagonally from lower left to upper right, depicts 293ST+SDGF-mTfR. The lower density map, having three lobes, depicts 293ST+SDGF-hTfR. The y-axis shows hTfR+Streptavidin from 10⁻⁴ to 10⁷, in increments of 10 on a log scale. The x-axis shows GFP from 0 to 10⁶, in increments of 10 on a log scale.

FIG. 4C illustrates that the peptide having a sequence of SEQ ID NO: 65, the peptide having a sequence of SEQ ID NO: 66, the peptide having a sequence of SEQ ID NO: 94, and the peptide having a sequence of SEQ ID NO: 96 bind human TfR. Data is displayed as four topographical density maps and indicates flow cytometry data using 293ST cells+SDGF-hTFR. Three density maps appear nearly superimposed and are oriented above a fourth density map. The lower density map is oriented horizontally and depicts SEQ ID NO: 65. The upper three density maps are oriented diagonally from lower left to upper right. The density map slightly above the other two corresponds to SEQ ID NO: 96. The density map slightly below the other two corresponds to SEQ ID NO: 66. The third density map corresponds to SEQ ID NO: 94. The y-axis shows hTfR+Streptavidin from 0 to 10⁷, in increments of 10 on a log scale. The x-axis shows GFP from 0 to 10⁶, in increments of 10 on a log scale.

FIG. 4D illustrates that the peptide having a sequence of SEQ ID NO: 65, the peptide having a sequence of SEQ ID NO: 66, the peptide having a sequence of SEQ ID NO: 94, and the peptide having a sequence of SEQ ID NO: 96 bind murine TfR. Data is displayed as four topographical density maps and indicates flow cytometry data using 293ST cells+SDGF-mTFR. Three density maps appear nearly superimposed and are oriented above a fourth density map. The lower density map is oriented horizontally and depicts SEQ ID NO: 65. The upper three density maps are oriented diagonally from lower left to upper right. The density map slightly above the other two corresponds to SEQ ID NO: 96. The density map slightly below the other two corresponds to SEQ ID NO: 66. The third density map corresponds to SEQ ID NO: 94. The y-axis shows hTfR+Streptavidin from 0 to 10⁷, in increments of 10 on a log scale. The x-axis shows GFP from 0 to 10⁶, in increments of 10 on a log scale.

FIG. 5 illustrates whole body autoradiography of mice injected intravenously with ¹⁴C-labeled TfR-binding peptides (SEQ ID NO: 65, SEQ ID NO: 66, SEQ ID NO: 94, and SEQ ID NO: 96) to assess biodistribution. Images were taken 3 hours after a dose of 100 nmol peptide (20 mg/kg, assuming approximately 25 g body weight) was administered to mice bearing xenograft tumors (subcutaneous human astrocytoma U87 cells). The images demonstrate substantial accumulation in spleen, liver, kidney, and also high accumulation in muscle, bone marrow, and skin. CNS accumulation is less than other tissues but still substantial (>25% of serum) compared to historical values of compounds and biologics that do not penetrate the blood-brain barrier (BBB)—background blood level found in brain is 3% that of cardiac blood signal.

FIG. 5A illustrates whole body autoradiography of mice injected intravenously with ¹⁴C-labeled SEQ ID NO: 65.

FIG. 5B illustrates whole body autoradiography of mice injected intravenously with ¹⁴C-labeled SEQ ID NO: 66.

FIG. 5C illustrates whole body autoradiography of mice injected intravenously with ¹⁴C-labeled SEQ ID NO: 94.

FIG. 5D illustrates whole body autoradiography of mice injected intravenously with ¹⁴C-labeled SEQ ID NO: 96.

FIG. 6 illustrates whole body autoradiography of mice injected intravenously with ¹⁴C-labeled peptides (SEQ ID NO: 65 and SEQ ID NO: 96) to assess biodistribution. Substantial accumulation in flank tumors was also seen. Accumulation in tumors is disperse throughout, rather than concentrated at sites rich in blood. This suggests extravasation and dispersal throughout the tumor parenchyma.

FIG. 6A illustrates whole body autoradiography of mice injected intravenously with ¹⁴C-labeled SEQ ID NO: 65.

FIG. 6B illustrates whole body autoradiography of mice injected intravenously with ¹⁴C-labeled SEQ ID NO: 65.

FIG. 6C illustrates whole body autoradiography of mice injected intravenously with ¹⁴C-labeled SEQ ID NO: 96.

FIG. 6D illustrates whole body autoradiography of mice injected intravenously with ¹⁴C-labeled SEQ ID NO: 96.

FIG. 7 illustrates the quantitation of peptide biodistribution and organ accumulation after a single 20 mg/kg IV dose of either a TfR-binding peptide having a sequence of SEQ ID NO: 65 or a peptide having a sequence of SEQ ID NO: 96. This dose achieved levels of >150 nM in the CNS, −1 μM in tumor after 3 hours. The bar graph quantifies the levels of each ¹⁴C-labeled peptides with known radioactivity in the same image and to peptide specific activity in various tissues from mice injected intravenously with peptides, 3 hours post-administration, as measured by whole body autoradiography.

FIG. 8 illustrates the quantitation of peptide biodistribution and organ accumulation for the peptides having a sequence of SEQ ID NO: 65 and SEQ ID NO: 96. The values are shown as levels in the tissue versus levels in the blood, where no brain penetration would give a level of 3% given that brain is 3% blood. The bar graph quantifies the levels of each ¹⁴C-labeled peptides normalized to controls with known radioactivity in the same image and to peptide specific activity in various tissues from mice injected intravenously with peptides, 3 hours post-administration, as measured by whole body autoradiography.

FIG. 9 illustrates scintillation counting over time in various tissues after administration of 20 mg/Kg of TfR-binding peptide having a sequence of SEQ ID NO: 96 with a specific activity of 147 Ci/mol, validating whole body autoradiography (WBA) tissue accumulation.

FIG. 9A illustrates scintillation counting over time in the kidneys. The y-intercept at time 0 (Y0) is 1900 pCi, the half-life (t_{1/2}) is 0.89 hrs, and the R² of the fit is 0.89.

FIG. 9B illustrates scintillation counting over time in the liver. The y-intercept at time 0 (Y0) is 400 pCi, the half-life (t_{1/2}) is 7.22 hrs, and the R² of the fit is 0.97.

FIG. 9C illustrates scintillation counting over time in the brain. The y-intercept at time 0 (Y0) is 7.3 pCi, the half-life (t_{1/2}) is 0.72 hrs, and the R² of the fit is 0.45.

FIG. 9D illustrates scintillation counting over time in the spleen. The y-intercept at time 0 (Y0) is 168 pCi, the half-life (t_{1/2}) is 12.1 hrs, and the R² of the fit is 0.93.

FIG. 9E illustrates scintillation counting over time in the muscle. The y-intercept at time 0 (Y0) is 31.4 pCi, the half-life (t_{1/2}) is 2.54 hrs, and the R² of the fit is 0.85.

FIG. 9F illustrates scintillation counting over time in the skin. The y-intercept at time 0 (Y0) is 68.3 pCi, the half-life (t_{1/2}) is 0.33 hrs, and the R² of the fit is 0.31.

FIG. 10 illustrates a serum elimination plot, including two-phase elimination regression analysis showing fast (95%, 15.6 min t_{1/2}) and slow (5%, 10.3 hr t_{1/2}) phase kinetics in mice intravenously administered 20 mg/kg of peptide having a sequence of SEQ ID NO: 96.

FIG. 11 illustrates multiple time regression analysis and capillary depletion analysis of TfR-binding peptides having a sequence of SEQ ID NO: 65 and SEQ ID NO: 96.

FIG. 11A illustrates multiple regression analysis of SEQ ID NO: 65 and SEQ ID NO: 96 in a single plot, wherein the y-axis indicates the brain to serum ratio.

FIG. 11B illustrates parenchyma and capillary distribution of TfR-binding peptides having a sequence of SEQ ID NO: 65 and SEQ ID NO: 96, wherein the y-axis indicates the tissue-to-serum ratio (μL/g).

FIG. 12 illustrates whole body autoradiography (WBA) of ¹⁴C peptide variant distribution. Histological sections of mice treated with ¹⁴C peptide variant are shown 30 minutes and 180 minutes post-administration. Representative brain and heart (inset) WBA images of three peptide generations, plus that of a control, non-TfR binding CDP are shown. Full body images of the same sections are shown in FIG. 13 and some of the 180 min. sections are also show in FIG. 5.

FIG. 13 illustrates the full body images of the whole-body radiography shown in FIG. 12, at 30 minutes (left column) and 180 minutes (right column) post-administration. Some of the sections from 180 min. are also shown in FIG. 5.

FIG. 14 illustrates CDP-NT (CDP-Neurotensin) peptide constructs which induce an IP₁ response downstream of the neurotensin receptor (NTSR) both in CRE-Luciferase (CRE-Luc) mice and in mammalian cells.

FIG. 14A illustrates the relevant pathways influencing CRE-driven luciferase in the CRE-Luc mice. PLC denotes phospholipase C. AC denotes adenylyl cyclase. CaMK denotes calmodulin-dependent protein kinase. CREB denotes the cAMP response element binding protein. PKA denotes protein kinase A. PDE denotes cAMP phosphodiesterase. FS denotes forskolin. Rol denotes rolipram. GPCR denotes a G-protein-coupled receptor.

FIG. 14B illustrates in vitro neurotensin (NT) receptor engagement showing IP₁ accumulation only in response to NT or NT peptide constructs in HEK-293 cells expressing NTSR1. IP₁ is measured using an assay kit (CisBio 62IPAPEB) with a readout of FRET ratio. N=3 wells for all except vehicle, which had N=36. Horizontal bar indicates sample mean. mTF=murine transferrin. Baseline HEK293=mean assay value for HEK293 cells (N=36 wells) that do not express NTSR1, included as a reference.

FIG. 15 illustrates immunohistochemistry stained tissue samples from mice administered NT peptide constructs. Tissue staining shows enhanced luciferase expression in the cortex, striatum, and thalamus of mice 4 hours after CDP-NT administration of SEQ ID NO: 138, SEQ ID NO: 139, and SEQ ID NO: 140. Mice administered Transferrin-NT (“Transferrin-NT,” SEQ ID NO: 345) or no peptide (“Unstimulated”) are shown as controls. Scale bar (top left panel) is 100 μm; all panels are the same magnification.

FIG. 16 illustrates quantitation of the effects of CDP-NT peptide constructs in CRE-luciferase (CRE-Luc) mice.

FIG. 16A illustrates luminescence (via intraperitoneal luciferin dosage) either before (unstimulated) or four hours after (denoted “NT fusion” or “Parent”) intravenous administration of parent TfR-binding peptides (“Parent”) (e.g., SEQ ID NO: 65, SEQ ID NO: 66, or SEQ ID NO: 96, or SEQ ID NO: 344 as denoted) or cystine dense peptide (CDP)-NT constructs or murine transferrin-NT constructs (e.g., comprising SEQ ID NO: 138-SEQ ID NO: 140 or SEQ ID NO: 345 as denoted) (“NT fusion”), using matched cohorts. Horizontal bar indicates sample mean. Significance was determined using a T-test (unpaired, 2-tailed). *: P<0.05. **: P<0.01. #: P<0.0001. For both the parent (SEQ ID NO: 344) and NT transferrin peptide constructs (SEQ ID NO: 345), murine transferrin was used.

FIG. 16B illustrates images from the SEQ ID NO: 96 cohort quantitated in FIG. 16A with pseudocolored luminescence intensity. All images were equally background-subtracted and contrast-enhanced for ease of viewing only.

FIG. 17 illustrates in vivo CRE-luciferase luminescence four hours after forskolin and rolipram induction. Images show luciferase fluorescence images of mice and serve as positive controls for the experiments depicted in FIG. 16. Forskolin and rolipram activate CRE-dependent luciferase expression independent of the neurotensin receptor pathway. Forskolin and rolipram induce CRE expression via activation of adenylyl cyclase and inhibition of cAMP phosphodiesterase, respectively. N=8 mice. Panels show replicate animals treated identically with rolipram and forskolin. One mouse was censored after the luciferin injection failed to hit the peritoneal cavity.

FIG. 18 illustrates luciferase expression in CRE-Luc mice after intravenous administration of free neurotensin peptide. Intravenous free neurotensin fails to induce luciferase expression in CRE-Luc mice. In vivo luminescence of CRE-luciferase mice 4 hours after intravenous administration of high dose free neurotensin peptide (300 nmol) or vehicle (10% DMSO in PBS) was assessed with intraperitoneal luciferin injection and imaged in an IVIS imager. “ns” indicates that the results were not significant (P=0.48), as calculated by T test (unpaired, 2 tailed).

FIG. 19 illustrates the quantitation of peptide biodistribution and organ accumulation after a single 20 mg/kg IV dose of either a TfR-binding peptide having a sequence of SEQ ID NO: 65 or a peptide having a sequence of SEQ ID NO: 96. Peptide levels are measured at 30 min and 3 hours (3 hour data is also shown in FIG. 7). This dose achieved levels of >150 nM in the CNS, −1 μM in tumor after 3 hours. The bar graph quantifies the levels of each ¹⁴C-labeled peptides with known radioactivity in the same image and to peptide specific activity in various tissues from mice injected intravenously with peptides, 3 hours post-administration, as measured by whole body autoradiography. Tissue data is plotted as seven clusters of four bars each. The 4-bar clusters correspond to different tissue types, from left to right, of Skin, Adipose, Muscle, Spleen, Kidney, Liver, Brain, and Blood, as shown on the x-axis. The four bars in each cluster correspond to, from left to right, SEQ ID NO: 65 30 min (N=10 sections), SEQ ID NO: 65 180 min (N=14 sections), SEQ ID NO: 96 30 min (N=12 sections), and SEQ ID NO: 96 180 min (N=21 sections). The y-axis shows nmol g⁻¹ tissue of peptide from 0.01 to 100 in increments of 10 on a log scale. The values of each bar, from left to right are, Skin: 1.33, 1.12, 1.70, 1.02, Adipose: 0.79, 0.52, 0.52, 0.27, Muscle: 0.63, 0.35, 0.44, 0.35, Spleen: 4.37, 20.03, 1.17, 3.60, Kidney: 29.05, 9.71, 16.91, 3.10, Liver: 4.79, 22.18, 1.80, 8.54, Brain: 0.23, 0.16, 0.21, 0.09, and Blood: 1.58, 0.62, 0.86, 0.34. The bars corresponding to Spleen, Kidney, and Liver are noticeably higher than the other sets. The inset shows the same data from the brain normalized to blood. The four bars correspond to, from left to right, SEQ ID NO: 65 30 min (N=10 sections), SEQ ID NO: 65 180 min (N=14 sections), SEQ ID NO: 96 30 min (N=12 sections), and SEQ ID NO: 96 180 min (N=21 sections). The y-axis shows peptide normalized to blood from 1 to 100 in increments of 10 on a log scale. The values for each bar, from left to right, are: 14%, 26%, 25%, and 27% of blood peptide levels.

FIG. 20 illustrates generation of a cleavable disulfide linkage between a peptide (e.g., a TfR-binding peptide of SEQ ID NO: 32) and a cyclic dinucleotide.

### DETAILED DESCRIPTION

Drug delivery of oligonucleotides to their intracellular targets is hampered by the ability to direct the oligonucleotide to the appropriate tissue or cell. In many cases, if an oligonucleotide is administered to a human or other subject, levels of oligonucleotide reaching the target tissue or cell may be inadequate to have a therapeutic effect. Furthermore, oligonucleotides that enter a target cell may be endocytosed by the target cells, and escape from the endosome may be needed in order to have the desired therapeutic effect. In some cases, complexing the oligonucleotide with sugars such as N-acetylgalactosamine (GalNAc) can enable delivery of adequate levels of oligonucleotide to hepatocyte of the liver. However, there exist few technologies to deliver oligonucleotides to other cells and tissues. Delivery of adequate levels to target cells can be enhanced by utilizing an endocytosing receptor present on the cell, such as transferrin receptor (TfR). TfR is present or upregulated on many cell types including those in muscle, spleen, bone marrow, GI tract, liver, and many tumors. TfR can in particular be used to deliver molecules to the central nervous system (CNS) by transcytosis across the blood-brain barrier (BBB), a term for the vascular endothelial cells in CNS capillaries.

The BBB system exists to prevent toxic metabolites and pathogens from entering the CNS (e.g., the brain), but also serves to render diseases of the CNS particularly difficult to treat using conventional medicines. It can be for this reason that primary CNS tumors (e.g., gliomas) and neuroinflammatory and neurodegenerative diseases such as Multiple Sclerosis and Alzheimer's disease respond particularly poorly to therapeutics that can otherwise be more effective in similar diseases affecting peripheral tissues where drug delivery to target cells can be less hindered. The myriad disorders of the CNS, from brain cancer to neurodegeneration to age-associated inflammatory processes, necessitate varied approaches to CNS drug delivery. While the BBB allows osmolytes and nutrients into the brain from serum, and CNS astrocytes provide many of the growth and survival signals required by neurons, several larger hormones and proteins such as transferrin (an iron chaperone), insulin, and leptin can cross the BBB.

CNS transport of larger molecules such as transferrin can be accomplished by receptor-mediated transcytosis or “vesicular transcytosis” (e.g., transport of cargo from the apical to the basal side, or vice versa, in intracellular vesicles). For example, variants of the endogenous receptors of insulin, leptin, and transferrin—InsR, ObR, and TfR, respectively—that contain the normal ectodomain can be employed by CNS vascular endothelial cells in order to facilitate transport of these molecules into the CNS. In this highly selective way, certain large molecules like transferrin (molecular weight is approximately 75 kDa) can access the brain parenchyma. Of these endogenous receptors, TfR is also highly expressed in certain tissues and tumors; such tissues or tumors are not protected by the BBB, but high TfR expression could permit selective accumulation of therapeutic agents if paired with an entity that binds TfR.

In various embodiments, the present disclosure provides compositions that enable transport of cargo molecules or active agents (e.g., oligonucleotides, small molecules, peptides, or proteins) across cell layers or barriers, including endothelial (e.g., the BBB) or epithelial cell layers and methods of using these compositions. In some embodiments, the present disclosure provides compositions that enable transport of oligonucleotides into cells by endocytosis. In some embodiments, the present disclosure provides compositions and methods that enable delivery of various molecules into the CNS that would otherwise not be able to pass the BBB or other cellular layers. TfR can in particular be used to deliver molecules to the central nervous system (CNS) that may be hampered by BBB, including enabling delivery by transcytosis. In some cases, the present disclosure provides compositions and methods for delivery of therapeutic and/or diagnostic molecules into the CNS, e.g., the brain. Thus, in various embodiments, the present disclosure provides peptides capable of causing endocytosis into cells, as well as peptides capable of crossing the BBB. These peptides can have an affinity and selectively for a transferrin receptor (TfR). The TfR-binding peptides can be cystine-dense peptides (CDPs). In some cases, the peptides of the present disclosure can deliver oligonucleotides into cells via TfR-mediated endocytosis. In some cases, the peptides of the present disclosure can cross the BBB via TfR-mediated transcytosis.

In some embodiments, the presently described peptides can be peptide conjugates, peptide constructs, fusion peptides, or fusion molecules such as linked by chemical conjugation of any molecule type, such as oligonucleotides, small molecules, peptides, or proteins, or by recombinant fusions of peptides or proteins, respectively (e.g., a peptide construct). The terms “fusion peptide” and “peptide fusion” are used interchangeably herein. A peptide of the present disclosure (e.g., a transferrin receptor targeting peptide) may form peptide complexes with another molecule, such as a small molecule, a nucleotide, a peptide, or a protein. For example, a peptide may form a peptide oligonucleotide complex comprising a peptide complexed with a nucleotide (e.g., a DNA or an RNA nucleotide molecule). In some embodiments, the peptide within a peptide oligonucleotide complex can be produced biologically or synthetically. Thus, in some cases, a TfR-binding peptide can comprise a TfR-binding peptide domain linked to another molecule or group of molecules such as nucleotides (e.g., oligonucleotides), small molecules, peptides, or proteins or other macromolecules such as nanoparticles.

In some embodiments, the present disclosure provides methods and compositions that enable transport to cells or tissues of interest or across cellular or molecular barriers. In various embodiments, the present disclosure provides methods and compositions that enable TfR-mediated transport across cellular layers (e.g., endothelial cells or epithelial cells) or cell membranes. In various embodiments, the present disclosure provides methods and compositions that enable TfR-mediated transport into cells, such as by endocytosis. In some cases, the TfR-binding peptides of the present disclosure enable transport across the blood brain barrier (BBB). In various aspects, the peptides of the present disclosure can be used to target any cell expressing TfR. In addition to the BBB, various other cells, tissues, and organs express TfR. Cells expressing TfR can include hepatocytes, erythrocytes and erythrocyte precursors in bone marrow, immune cells, stem cells, and rapidly dividing cells. Tissues and organs expressing TfR can include the brain (e.g., cerebral cortex, hippocampus, caudate, cerebellum), endocrine tissues (e.g., thyroid, parathyroid, and adrenal glands), bone marrow and immune system (e.g., appendix, lymph node, tonsil, spleen), muscle tissues (e.g., heart, skeletal, and smooth muscle), liver, gallbladder, pancreas, gastrointestinal tract (e.g., oral mucosa, esophagus, stomach, duodenum, small intestine, colon, rectum), kidney, urinary bladder, female tissues (e.g., fallopian tube, breast, vagina, cervix, endometrium, ovary, and placenta), adipose and soft tissue, and skin. Thus, the TfR-binding peptides of the present disclosure can be used to target these cells, tissues, and organs and deliver an active agent to these cells, tissues, and organs via, for example, TfR-mediated transcytosis (e.g., across cellular barrier such as the BBB) or TfR-mediated endocytosis (e.g., across cell membranes into cells).

In various embodiments, the present disclosure provides methods and compositions that enable TfR-mediated transport and delivery to cancer cells expressing TfR or CMYC-overexpressing cancers, as, in some cases, CMYC-overexpression can cause TfR-overexpression. Cancers overexpressing TfR can include ovarian cancer, colon cancer, lung cancer, cancer located in the bone or bone marrow, glioblastoma, astrocytoma, glioma, medulloblastoma, ependymoma, choroid plexus carcinoma, midline glioma, diffuse intrinsic pontine glioma (DIPG), breast cancer, liver cancer, colon cancer, brain cancer, spleen cancer, cancers of the salivary gland, kidney cancer, muscle cancers, bone marrow cell cancers, skin cancer, genitourinary cancer, osteosarcoma, muscle-derived sarcoma, melanoma, head and neck cancer, neuroblastoma, prostate cancer, bladder cancer, acute lymphocytic leukemia, acute myeloid leukemia, chronic lymphocytic leukemia, chronic myeloid leukemia, Hodgkin lymphoma, Non-Hodgkin lymphoma, or a CMYC-overexpressing cancer.

In some embodiments, the presently described peptides (e.g., a peptide within the peptide oligonucleotide complex), comprise one or more TfR-binding peptides as described herein conjugated to, linked to, or fused to one or more target-binding agents (e.g., a nucleotide target-binding agent), one or more active agents (e.g., a therapeutic agent), one or more detectable agents, or combinations thereof. Peptide oligonucleotide complexes as described herein can include chemical conjugates and recombinant fusion molecules. In some cases, a chemical conjugate can comprise a TfR-binding peptide as described herein that is chemically conjugated to or linked to another molecule. Molecules can include small molecules, peptides, polypeptides, proteins, or other macromolecules (e.g., nanoparticles) and polymers (e.g., nucleic acids, polylysine, or polyethylene glycol). For example, a peptide oligonucleotide complex may comprise a TfR-binding peptide chemically conjugated or linked to a nucleic acid molecule (e.g., a DNA or RNA molecule). In some cases, a TfR-binding peptide of the present disclosure is conjugated to another molecule via a linker. Linker moieties can include cleavable (e.g., pH sensitive or enzyme-labile linkers) or stable linkers. In some embodiments, a peptide construct is a fusion molecule (e.g., a fusion peptide or fusion protein) that can be recombinantly expressed, and wherein the fusion molecule can comprise one or more TfR-binding peptides fused to one or more other molecules peptides, polypeptides, proteins, or other macromolecules that can be recombinantly expressed.

In some cases, a TfR-binding peptide of the present disclosure is conjugated to, linked to, or fused to a nucleotide of the present disclosure, thereby forming a TfR-binding peptide oligonucleotide complex. In some embodiments, the nucleotide of the peptide oligonucleotide complex may be a target-binding agent capable of exerting a biological effect on a target molecule or functioning as an active, therapeutic, or diagnostic agent. In some embodiments, an additional active agent (e.g., a small molecule, peptide, or protein active agent) cargo molecule may be conjugated to, linked to, or fused to the peptide oligonucleotide complex. An active agent may be a therapeutic agent or a detectable agent. A therapeutic agent may be capable of exerting a certain biological effect, and a detectable agent may function as a diagnostic molecule. The resulting in peptide oligonucleotide-active agent conjugate may be directed to a certain biological effect. In some cases, the therapeutic or diagnostic cargo molecules used herein, or the nucleotide cargo molecules, can be transported across the BBB via TfR-mediated transcytosis, or to other cells via TfR-mediated endocytosis, to reach the CNS or other TfR-rich environment. Once inside the CNS or other TfR-rich environment, the cargo molecules can target a specific cell, cell population, or tissue. In some cases, the therapeutic or diagnostic cargo molecules are known compounds that, when used in combination with the compositions and methods disclosed herein, are able to exert a significantly higher potency inside the CNS due to effective transport across the BBB.

The peptides of the present disclosure, or derivatives, fragments, or variants thereof, can have an affinity and selectively for TfR, or a derivative or analog thereof. In some cases, the peptides of the present disclosure can be engineered using site-saturation mutagenesis (SSM) to exhibit improved TfR-binding properties or promote transcytosis more effectively. In some cases, the peptides of the present disclosure are cystine-dense peptides (CDPs), related to knotted peptides or hitchin-derived peptides or knottin-derived peptides. The TfR-binding peptides can be cystine-dense peptides (CDPs). The terms “peptides”, “CDPs”, “TfR-binding peptides,” “TfR-binding CDPs,” and “TfR-binding peptides” are used interchangeably herein. Hitchins can be a subclass of CDPs wherein six cysteine residues form disulfide bonds according to the connectivity [1-4], 2-5, 3-6 indicating that the first cysteine residue forms a disulfide bond with the fourth residue, the second with the fifth, and the third cysteine residue with the sixth. The brackets in this nomenclature indicate cysteine residues form the knotting disulfide bond. (See e.g., Correnti et al. Screening, large-scale production, and structure-based classification for cystine-dense peptides. Nat Struct Mol Biol. 2018 March; 25(3): 270-278). Knottins can be a subclass of CDPs wherein six cysteine residues form disulfide bonds according to the connectivity 1-4, 2-5, [3-6]. Knottins are a class of peptides, usually ranging from about 20 to about 80 amino acids in length that are often folded into a compact structure. Knottins are typically assembled into a complex tertiary structure that is characterized by a number of intramolecular disulfide crosslinks and may contain beta strands and other secondary structures. The presence of the disulfide bonds gives knottins remarkable environmental stability, allowing them to withstand extremes of temperature and pH and to resist the proteolytic enzymes of the blood stream. In some cases, the peptides described herein can be derived from knotted peptides. The amino acid sequences of peptides as disclosed herein can comprise a plurality of cysteine residues. In some cases, at least cysteine residues of the plurality of cysteine residues present within the amino acid sequence of a peptide participate in the formation of disulfide bonds. In some cases, all cysteine residues of the plurality of cysteine residues present within the amino acid sequence of a peptide participate in the formation of disulfide bonds. As described herein, the term “knotted peptide” can be used interchangeably with the terms “cystine-dense peptide”, “CDP”, or “peptide”.

Provide herein are methods of identification, maturation, characterization, and utilization of CDPs that bind the transferrin receptor and allow accumulation of bioactive molecules, including oligonucleotides, at therapeutically relevant concentrations in a subject (e.g., a human or non-human animal). This disclosure demonstrates the utility of CDPs as a diverse scaffold family that can be screened for applicability to modern drug discovery strategies. CDPs comprise alternatives to existing biologics, primarily antibodies, which may bypass some of the liabilities of the immunoglobulin scaffold, including poor tissue permeability, immunogenicity, proteolytic susceptibility, and long serum half-life that can become problematic if toxicities arise. Peptides of the present disclosure in the 20-80 amino acid range represent medically relevant therapeutics that are mid-sized, with many of the favorable binding specificity and affinity characteristics of antibodies but with improved stability, reduced immunogenicity, and simpler manufacturing methods, including the potential for chemically synthetic production and chemical conjugation. The intramolecular disulfide architecture of CDPs provides particularly high stability metrics, reducing fragmentation and immunogenicity, while their smaller size could improve tissue penetration or cell penetration and facilitate tunable serum half-life. Disclosed herein are peptides representing candidate peptides that can serve as CNS drug delivery vehicles, as oligonucleotide drug delivery vehicle, or both. A peptide of the present disclosure may be a cell-penetrating peptide (CPP). A CPP may be cell-penetrating, tissue-penetrating, or both.

In some embodiments, a CPP may be a CDP. CPPs may comprise peptides that facilitate cellular intake, uptake, endocytosis, or endosomal release of various moieties and agents, and themselves may translocate across a cell membrane. CPPs may contain protein transduction domains, which are a class of short peptide sequences which can translocate across the cell membrane. A cell-penetrating peptide may directly or indirectly enter the endosome of a cell, the cytosol of a cell, the nucleus of a cell, or other subcellular locations of a cell. A cell-penetrating peptide can be used as an appropriate carrier for various cargos including nucleic acids, peptides, proteins, small interfering RNA (siRNA), dsRNA, micro RNA (miRNA, or miR), antisense RNA, antisense oligonucleotides, complementary RNA or DNA, interfering RNA, small nuclear RNA (snRNA), spliceosomal RNA, nucleotide sequence is single stranded (e.g., ssDNA or ssRNA) or double stranded (e.g., dsDNA or dsRNA) or a combination of single and double stranded (for example with a mismatched sequence, hairpin or other structure), an antisense RNA, complementary RNA, inhibitory RNA, interfering RNA, nuclear RNA, oligonucleotides complementary to antisense oligonucleotide (ASO), microRNA (miRNA), an oligonucleotide complementary to natural antisense transcripts (NATs) sequences, siRNA, snRNA, aptamer, gapmer, anti-miR, splice blocker ASO, or U1 Adapter, any of the foregoing nucleic acids with alternative backbone chemistries (e.g., 2′ substitutions of the ribose sugar group, linked nucleic acids (LNAs), peptide nucleic acids (PNAs), or morpholinos), radionuclides, imaging agents, fluorescent agents, additional therapeutic agents, nanoparticles, and the like. A cargo may be a nucleotide target-binding agent, an active agent, a therapeutic agent, a detectable agent, or combinations thereof.

An example of a cell-penetrating peptide functioning as a carrier for a cargo is a peptide oligonucleotide complex comprising TfR-binding peptide carrier and a nucleotide cargo (e.g., an oligonucleotide). In some embodiments, the nucleotide cargo of a TfR-binding peptide oligonucleotide complex may comprise a small interfering RNA (siRNA), dsRNA, micro RNA (miRNA, or miR), antisense RNA, antisense oligonucleotides, complementary RNA or DNA, interfering RNA, small nuclear RNA (snRNA), spliceosomal RNA, nucleotide sequence is single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to natural antisense transcripts (NATs) sequences, siRNA, snRNA, aptamer, gapmer, anti-miR, splice blocker ASO, or U1 Adapter, or any other nucleic acid molecule. In some embodiments, the TfR-binding peptide of the TfR-binding peptide oligonucleotide complex may comprise an amino acid sequence set forth in any one of SEQ ID NO: 1-SEQ ID NO: 134 or SEQ ID NO: 306-SEQ ID NO: 335.

CPPs can be an artificial or engineered sequence (e.g., a synthetic sequence). CPPs may comprise multiple protein sequences, whether such protein sequences are native, synthetic, or a variant (e.g., chimeric). CPPs can be derived from a single protein sequence, whether such protein sequences are native synthetic or variant (e.g., a protein-derived sequence). CPPs can exhibit a variety of physiochemical properties such as cationic, amphipathic, or hydrophobic. Some mechanisms for internalization of CPPs include direct cell penetration, use of the endocytosis pathway, and translocation through the formation of a transitory structure. It is understood that the description and use of a cell-penetrating peptide (CPP) herein is non-limiting. Non-limiting examples of CPPs can be found, for example, in Derakhshankhah and Jafari, “Cell penetrating peptides: A concise review with emphasis on biomedical applications” (Biomedicine & Pharmacotherapy; Volume 108, December 2018, Pages 1090-1096), which is incorporated by reference in its entirety.

Some therapeutic molecules that can be used in combination with the herein disclosed methods and compositions can be able to target one or more specific cells or tissues that are part of the CNS. For example, the neurotensin receptor (NTSR), a G-protein coupled receptor, is targeted with a fusion protein comprising a CDP linked to neurotensin (abbreviated herein as “NT”; ELYENKPRRPYIL (SEQ ID NO: 341)), or a derivative thereof, a neuropeptide that activates the NTSR. In some cases, a CDP-NT peptide construct is used to prevent or treat chronic pain or neuropathic pain in a subject (e.g., a human).

Also described herein are peptides that selectively home, target, are directed to, migrate to, are able to reach, are retained by, or accumulate in and/or bind to specific regions, tissues, structures or cells of the central nervous system (CNS) that are involved in sensing, modulating, managing, decreasing, ablating or reducing pain, including nociceptive pain, or other therapeutic indications as described herein. A peptide that homes, targets, migrates to, is directed to, is retained by, or accumulates in and/or binds to one or more specific regions, tissues, structures or cells of the affected region can have fewer off-target and potentially negative effects, for example, side effects that often limit use and efficacy of pain drugs. In addition, such peptides can deliver active agents to regions, such as the CNS, where those active agents are otherwise unable to reach the CNS at therapeutic levels, such as due to the blood-brain barrier. Such peptides can also reduce need for other pain medication, including opioid medications. In addition, such peptides can reduce dosage and increase the efficacy of existing drugs by directly targeting them to a specific region, tissue, structure or cell of the affected region and helping to contact the affected region or increasing the local concentration of agent. The peptide itself can modulate pain or it can be conjugated to an agent that modulates pain. Such pain modulation may operate by various mechanisms such as modulating inflammation, autoimmune responses, direct or indirect action on pain receptors, cell killing, or programmed cell death (whether via an apoptotic and/or non-apoptotic pathway of diseased cells or tissues, and the like (Tait et al. J Cell Sci 127(Pt 10):2135-44 (2014)).

Also described herein are peptides that selectively home, target, are directed to, migrate to, are able to reach, are retained by, or accumulate in and/or bind to cells expressing TfR. Cells expressing TfR can include hepatocytes, erythrocytes and erythrocyte precursors in bone marrow, immune cells, stem cells, tumor cells, and rapidly dividing cells. Tissues and organs expressing TfR can include the brain (e.g., cerebral cortex, hippocampus, caudate, cerebellum), endocrine tissues (e.g., thyroid, parathyroid, and adrenal glands), bone marrow and immune system (e.g., appendix, lymph node, tonsil, spleen), muscle tissues (e.g., heart, skeletal, and smooth muscle), liver, gallbladder, pancreas, gastrointestinal (GI) tract (e.g., oral mucosa, esophagus, stomach, duodenum, small intestine, colon, rectum), kidney, urinary bladder, female tissues (e.g., fallopian tube, breast, vagina, cervix, endometrium, ovary, and placenta), male tissues (e.g., the testes, conducting tubules and ducts (epididymis, vas deferens, ejaculatory ducts), accessory sex glands (seminal vesicles, prostate, and bulbourethral glands), adipose and soft tissue, liver, skin and tumor cells.

CDPs may be advantageous for delivery to the CNS, or other TfR-expressing tissues, as compared to other molecules such as antibodies due to smaller size, greater tissue or cell penetration, and quicker clearance from serum, and as compared to antibodies or smaller peptides due to resistance to proteases (both for stability and for immunogenicity reduction). In some embodiments, the TfR-binding peptides of the present disclosure (e.g., CDPs, knotted peptides, or hitchins), TfR-binding peptide conjugates (e.g., comprising one or more TfR-binding peptides and one or more active agents, including oligonucleotide active agents), or engineered TfR-binding fusion peptides (e.g., comprising one or more TfR-binding peptides and one or more peptides) may have properties that are superior to TfR-binding antibodies. For example, the peptides described herein (e.g., peptides within a peptide oligonucleotide complex) can provide superior, deeper, and/or faster tissue or cell penetration to cells and targeted tissues (e.g., brain parenchyma penetration, solid tumor penetration) and faster clearance from non-targeted tissues and serum. The TfR-binding peptides, TfR-binding peptide conjugates, or TfR-binding fusion peptides of this disclosure may have lower molecular weights than TfR-binding antibodies. The lower molecular weight may confer advantageous properties on the TfR-binding peptides, TfR-binding peptide conjugates, or TfR-binding fusion peptides of this disclosure as compared to TfR-binding antibodies. For example, the TfR-binding peptides, TfR-binding peptide conjugates, or TfR-binding fusion peptides of this disclosure may penetrate a cell or tissue more readily than an anti-TfR antibody or may have lower molar dose toxicity than an anti-TfR antibody. The TfR-binding peptides, TfR-binding peptide conjugates, or TfR-binding fusion peptides of this disclosure may be advantageous for lacking the Fc function of an antibody. The TfR-binding peptides, TfR-binding peptide conjugates, or TfR-binding fusion peptides of this disclosure may be advantageous for allowing higher concentrations, on a molar basis, of formulations, including lower viscosity formulations or formulations that may be delivered in a smaller volume such as subcutaneously, intramuscular, intravitreally, and intrathecally.

The TfR-binding peptides, TfR-binding peptide conjugates, TfR-binding peptide complexes, or TfR-binding fusion peptides of this disclosure may have a wider therapeutic window (e.g., the dosage above which a therapeutic pharmacodynamic response is observed but below which toxicity is observed) as compared to TfR-binding antibody-based therapeutics. The TfR-binding peptides, TfR-binding peptide conjugates, or TfR-binding fusion peptides of this disclosure may be used at higher molar dosage with less risk of toxicity as compared to TfR-binding antibody-based therapeutics. The TfR-binding peptides, TfR-binding peptide conjugates, or TfR-binding fusion peptides of this disclosure may have fewer epitopes to trigger an adaptive immune response, resulting in reduced immunogenicity as compared to TfR-binding antibody-based therapeutics. The TfR-binding peptides, TfR-binding peptide conjugates, or TfR-binding fusion peptides of this disclosure may exhibit more facile and less disruptive incorporation of active agents into protein fusion when used within the peptide oligonucleotide complex as compared to TfR-binding antibody-based therapeutics. The TfR-binding peptide oligonucleotide complexes of this disclosure may have a smaller surface area, resulting in lower risk for off-target binding, as compared to TfR-binding antibody-based therapeutics. An exemplary comparison is described in EXAMPLE 49.

In some embodiments, the TfR-binding peptide oligonucleotide complexes of this disclosure exhibit lower on-target toxicity than an anti-TfR antibody when administered to a subject at the same molar dose or at a similarly effective dose. In some embodiments, the TfR-binding peptide oligonucleotide complexes exhibit lower off-target toxicity than an antibody when administered to a subject at the same molar dose or a similarly effective dose. For example, the TfR-binding peptides, TfR-binding peptide conjugates, or TfR-binding fusion peptides of this disclosure may be administered to a subject at about 1-fold, 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, 100-fold higher molar dose than an antibody while providing similar or lower observed toxicity. In some embodiments, the TfR-binding peptides, TfR-binding peptide conjugates, or TfR-binding fusion peptides of this disclosure exhibit higher efficacy than an anti-TfR antibody when administered to a subject at the same dose by weight as the anti-TfR antibody. The TfR-binding peptides of the present disclosure, when fused to a half-life extending moiety (e.g., Fc, SA21, PEG), can be delivered at even lower doses while preserving activity and efficacy and, thus, is far superior to administering an anti-TfR antibody.

In some embodiments, the present disclosure provides peptides (e.g., CDPs, knotted peptides, or hitchins), chemical conjugates (e.g., comprising one or more TfR-binding peptides and one or more active agents), or recombinantly expressed fusion molecules (e.g., comprising one or more TfR-binding peptides and one or more active agents, including oligonucleotide active agents) that bind to TfR. The TfR-binding peptides can be cystine-dense peptides (CDPs). The terms “peptides”, “CDPs”, “TfR-binding peptides,” “TfR-binding CDPs,” “TfR-binding peptides,” and “engineered TfR-binding peptides” are used interchangeably herein. The binding of peptides described in the present disclosure to TfR can facilitate transcytosis of the peptide oligonucleotide complex (e.g., a complex comprising an oligonucleotide active agent fused, conjugated, or linked to a TfR-binding peptide) across a cell barrier (e.g., the BBB) or can also facilitate endocytosis of the peptide oligonucleotide complex into a cell. In some embodiments, TfR-binding CDPs are identified and binding can be determined by crystallography. Peptides of the present disclosure can have cross-reactivity across species. For example, the peptides disclosed herein, in some cases, bind to human and murine TfR. Peptide oligonucleotide complexes disclosed herein can accumulate in the CNS and can penetrated the BBB via engagement of the TfR, following intravenous administration, including induction of a cAMP response element signaling cascade using neurotensin peptide constructs. Disclosed herein are TfR-binding CDPs for use within the peptide oligonucleotide complex as therapeutic delivery agents in oncology, autoimmune disease, acute and chronic neurodegeneration, pain management, inflammatory disease, immune disease, infectious disease, cardiovascular disease, or any other disease. Delivery of active or pharmaceutical agents, such as the nucleotide of a peptide oligonucleotide complex, via TfR-binding CDP can be advantageous over conventional anti-TfR antibodies due to simpler manufacturing (peptides can be made via biologic or synthetic means), improved stability, and smaller size (less potential for steric hindrance of cargo activity). Thus, the methods and compositions of the present disclosure can provide a solution to the problem of effectively transporting cargo molecules (e.g., therapeutic and/or diagnostic small molecules, peptides or proteins) into the CNS (e.g., the brain) or into other cells or tissues. For example, the peptides of the present disclosure aid in drug delivery to tumors located in the brain.

In some embodiments of the present disclosure, a diverse library of CDPs, knotted peptides, hitchins, or peptides derived from knotted peptides or hitchins can be used in combination with a mammalian surface display screening platform is used to identify peptides that specifically bind to human TfR. (See e.g., Crook et al. (2017) Mammalian display screening of diverse cystine-dense peptides for difficult to drug targets. Nat Commun 8:2244). In some embodiments, a diverse library of CDPs, knotted peptides, hitchins, or peptides derived from knotted peptides or hitchins is mutagenized from endogenous peptide sequences to provide novel peptide sequences. Once TfR-binding peptides have been identified, affinity maturation (e.g., site-saturation mutagenesis) can be performed to produce an allelic series of binders with varying (e.g., improved) affinities for TfR. These techniques can be used in combination with various other analytical methods (e.g., crystallography or spectroscopy) in order to determine the nature of peptide-receptor interaction (e.g., critical amino acid residues for receptor binding etc.). In some cases, the peptides of the present disclosure are developed to bind human TfR; however, those peptides can show cross-reactivity with TfR derived from other species such as murine TfR.

The peptides of the present disclosure can have varying biodistribution patters in vivo. In some cases, mouse biodistribution and pharmacokinetic studies are performed using, for example, radiolabeled peptides (e.g., ¹⁴C-labeled peptides), in order to determine organ distribution, the uptake and residence times of the peptides in target organs (e.g., brain), and their mode of clearance (e.g., renal or hepatic).

In various embodiments, the present disclosure provides peptides and peptide oligonucleotide complexes that accumulate in the CNS (e.g., the brain). In some cases, CNS accumulation of those peptides or complexes is due to penetration across the BBB. In some cases, engineered TfR-binding peptide variants as described herein cross the BBB by TfR-mediated transcytosis. In some cases, the TfR-binding peptides of the present disclosure accumulate in tumor tissue that is located in the CNS. In some cases, the tumor that the peptide accumulates in is a brain tumor (e.g., glioma). In some cases, the present disclosure provides peptides and peptide oligonucleotide complexes that accumulate in tissues or cells that express TfR, including hepatocytes, erythrocytes and erythrocyte precursors in bone marrow, immune cells, stem cells, and rapidly dividing cells, the brain (e.g., cerebral cortex, hippocampus, caudate, cerebellum), endocrine tissues (e.g., thyroid, parathyroid, and adrenal glands), bone marrow and immune system (e.g., appendix, lymph node, tonsil, spleen), muscle tissues (e.g., heart, skeletal, and smooth muscle), liver, gallbladder, pancreas, gastrointestinal tract (e.g., oral mucosa, esophagus, stomach, duodenum, small intestine, colon, rectum), kidney, urinary bladder, female tissues (e.g., fallopian tube, breast, vagina, cervix, endometrium, ovary, and placenta), adipose and soft tissue, and skin and tumors including solid tumors. As described herein, the terms “accumulates” and “accumulates in” can be used interchangeably and be used to refer to “accumulation in or accumulation on a cell and/or a tissue and can be understood as a gradual increase of concentration of the respective molecule (e.g., a TfR-binding peptide oligonucleotide complex) over time.

In some embodiments, the engineered peptides of the present disclosure (e.g., histidine-containing or histidine-enriched TfR-binding peptides) can have a high TfR binding affinity at physiological pH but a significantly reduced binding affinity at lower pH levels such as endosomal pH of 5.4. In some cases, the TfR-binding peptides of the present disclosure can be optimized for improved intra-vesicular (e.g., intra-endosomal), cytosolic, nuclear, and/or intracellular delivery function while retaining high TfR binding capabilities. In some cases, histidine scans and comparative binding experiments can be performed to develop and screen for such peptides. In addition, some peptides can comprise (e.g., conjugated to, linked to, or fused to) a motif that facilitates low-pH endosomal escape of the peptide oligonucleotide complex, or of the nucleotide after release from the peptide such as upon cleavage of a cleavable linker, for enhanced delivery functions (e.g., intracellular delivery of an oligonucleotide therapeutic agent or additional therapeutic agent). In some embodiments, an amino acid residue in a peptide of the present disclosure is substituted with a different amino acid residue to alter a pH-dependent binding affinity to TfR. The amino acid substitution may increase a binding affinity at low pH, increase a binding affinity at high pH, decrease a binding affinity at low pH, decrease a binding affinity at high pH, or a combination thereof.

Exemplary peptides of the present disclosure are shown in TABLE 1 with amino acid sequences set forth in SEQ ID NO: 1-SEQ ID NO: 128. Additional exemplary peptides of the present disclosure include peptides with amino acid sequences set forth in SEQ ID NO: 129—SEQ ID NO: 134, shown in TABLE 12, and SEQ ID NO: 306-SEQ ID NO: 335.

In various embodiments, the peptides of the present disclosure are part of a peptide oligonucleotide complex, wherein the nucleotide comprises an antisense RNA, complementary RNA, inhibitory RNA, interfering RNA, nuclear RNA, an oligonucleotide complementary to antisense oligonucleotide (ASO), microRNA (miRNA), natural antisense transcripts (NATs) sequences, siRNA, snRNA, aptamer, gapmer, anti-miR, splice blocker ASO, or U1 Adapter, designed to transport nucleotides or additional therapeutic and/or diagnostic molecules (e.g., small molecules, peptides, or proteins) across cell layers or cell barriers such as the BBB. Exemplary peptide oligonucleotide complexes contain a nucleic acid portion that comprises an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter that act as a targeting agent against a target gene or mRNA, for example, to a reference target as shown in TABLE 5 or TABLE 6, EXAMPLE 50-EXAMPLE 54 or as otherwise described, and with or without also comprising a U1 Adapter as shown in TABLE 7.

A peptide within the peptide nucleic acid complex of the present disclosure can comprise a TfR targeting peptide linked (e.g., chemically conjugated or fused) to a therapeutic and/or diagnostic molecule or compound, which may be the nucleotide or may be an additional agent such as a small molecule, peptide, or protein. The TfR-binding peptide oligonucleotide complex can cross the BBB via TfR-mediated transcytosis and hence deliver the therapeutic and/or diagnostic molecule or compound into the CNS. The TfR-binding peptide-oligonucleotide complex construct can bind to TfR and be endocytosed into cells and hence deliver the therapeutic and/or diagnostic molecule or compound into the cell. The methods and compositions of the present disclosure therefore can have a profound impact on the diagnosis, prevention, and treatment of various diseases or conditions. Disease areas for which the methods and compositions of the present disclosure can be used include, but are not limited to, oncology, autoimmune diseases, acute and chronic neurodegeneration, muscle disorders, neuropsychological disorders, epilepsy, schizophrenia, depression, anxiety, bipolar disorder, developmental brain disorders (e.g., autism spectrum), pain, epilepsy, mood disorder, and pain management. Disease areas for which the methods and compositions of the present disclosure can be used also include, immune and inflammatory diseases, cardiovascular diseases, infectious disease, ocular diseases, genetic diseases.

Hence, a subject having a disorder of the CNS or other tissue that is otherwise unable to benefit from a drug or drug class whose limited by poor BBB penetration or unable to benefit from a drug that cannot be delivered to the relevant cell type at therapeutic levels, can benefit from the compositions and methods described herein because therapeutically effective concentrations of the drug, such as the nucleotide of the peptide oligonucleotide complex, in the CNS or other target cell type can be achieved by conjugation to a TfR-binding peptide provided herein. In addition to pharmacological advances, the peptide oligonucleotide complex compositions and methods disclosed herein comprising TfR-binding peptides can be superior over conventional CNS- and TfR-targeting agents (e.g., anti-TfR antibodies) in terms of their production, quality control, and safety. For example, the peptide oligonucleotide complexes offer a more resource-effective manner (e.g., peptides can be synthesized via biologic or synthetic approaches); the peptide oligonucleotide complexes of the present disclosure can show improved ex vivo and in vivo stability, are smaller in size (e.g., less potential for steric hindrance of cargo activity and the ability to penetrate dense tissue such as solid tumors as well as the potential for faster clearance from systemic circulation), exhibit a higher tissue or cell penetration, and have lower immunogenicity. Peptide oligonucleotide complexes of this disclosure can be engineered to have lower immunogenicity by combining crystallographic data with major histocompatibility complex (MHC) or human leukocyte antigen (HLA) peptide fragment binding experiments or computational predictions thereof, or a combination of the foregoing. The peptide oligonucleotide complexes of the present disclosure can be constructed more readily by fully synthetic production or by site specific conjugation that may be use nonnatural amino acids, a single reactive amino acid, or selective deprotection. They can also be constructed more readily by the use of organic solvents or heat or pH that may damage an antibody including its folding structure. The peptide oligonucleotide complexes of the present disclosure may be more readily manufactured to be more pure, more safe, less immunogenic, less toxic, more potent, or have a lower cost of goods.

The identification of therapeutic or diagnostic agents that have the ability to cross cellular layers or barriers such as the BBB have been challenging, as such methods involve demand for high specificity or targeting, high affinity binders for receptors that can promote transcytosis (e.g., TfR), and the ability to target cells and act on target proteins (low off-target adverse effects) after transcytosis. With few exceptions, high throughput screening campaigns with small molecule libraries failed to provide specific compounds capable of crossing endothelial or epithelial layers and/or transporting cargo across those layers.

Described herein are, in some embodiments, peptide oligonucleotide complexes and methods of screening for peptides within the peptide oligonucleotide complexes that target a protein of interest, such as TfR. Compared to wildtype or endogenous molecules such as transferrin, the methods and compositions as described herein can provide peptides within the peptide oligonucleotide complex with improved TfR-binding capabilities, or peptides within the peptide oligonucleotide complex that exhibit improved transport capabilities across the BBB or exhibit improved transport into cells such as by endocytosis, or any combination thereof. In some cases, the presently described peptides within the peptide oligonucleotide complex efficiently transport cargo molecules (e.g., the nucleotide or additional therapeutic or diagnostic small molecules or proteins) across endothelial cell layers (e.g., the BBB) or epithelial layers or into cells by endocytosis. In some embodiments, the TfR-binding peptides within the peptide oligonucleotide complex of the present disclosure bind to a TfR and promote vesicular transcytosis. In some cases, the TfR-binding peptides within the peptide oligonucleotide complex of the present disclosure bind to a cell that overexpress a TfR (e.g., a cancer cell, an immune cell, a hematopoietic cell, an endothelial cell, an epithelial cell, a hepatocyte, a myocyte, a cardiomyocyte, or a retinal cell) and promotes uptake of the peptide by the cell. In some aspects, a TfR binding peptide oligonucleotide complex as described herein promotes vesicular transcytosis and uptake by a TfR-overexpressing cell such as a cancer, or a combination thereof.

The TfR-binding peptides within the peptide oligonucleotide complex of the present disclosure can bind TfR of different species including human, monkey, mouse, and rat TfR. In some cases, variations or mutations in any of the amino acid residues of a TfR-binding peptide within the peptide oligonucleotide complex may influence cross-reactivity. In some cases, variations or mutations in any of the amino acid residues of a TfR-binding peptide within the peptide oligonucleotide complex that interact with the bindings site of TfR may influence cross-reactivity.

Described herein are peptides within the peptide oligonucleotide complex, including, but not limited to, designed or engineered peptides, recombinant peptides, and cystine-dense peptides (CDPs)/small disulfide-knotted peptides (e.g., knotted peptides, hitchins, and peptides derived therefrom), that can be large enough to carry a cargo molecule while retaining the ability to bind a target protein with high affinity (e.g., TfR), but yet small enough to access cellular compartments, such as the cytosol or the nucleus, or tissues, such as the center of cell agglomerates (e.g., solid tumors). In some cases, the peptides as described herein carry cargo molecules across the BBB into the CNS (e.g., the parenchyma) via vascular transcytosis. In some cases, the transcytosis is TfR-mediated.

Further described herein are methods and compositions for determining the nature of peptide-receptor interactions (e.g., using X-ray crystallography) as well as their pharmacodynamic and pharmacokinetic properties in vivo, including accumulation in the CNS (e.g., brain). Some of the peptides within the peptide oligonucleotide complex described herein have the ability to target and accumulate in tumor cells. In some cases, the tumor cells overexpress TfR. In some aspects, the peptides of the present disclosure have high in vivo stabilities, e.g., high protease stability, high tolerability of reducing agents such as glutathione (GSH), and tolerate elevated temperatures (e.g., up to 95° C.).

The present disclosure provides, in some embodiments, a peptide or protein design approach based on the 3D protein or receptor structure for identifying peptides or proteins capable of binding such receptor. In some cases, the receptor is a transferrin receptor.

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R).

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

The terms “peptide”, “polypeptide”, “protein”, “hitchin”, “cystine-dense peptide”, “knotted peptides” or “CDP” can be used interchangeably herein to refer to a polymer of amino acid residues. In various embodiments, “peptides”, “polypeptides”, and “proteins” can be chains of amino acids whose alpha carbons are linked through peptide bonds. The terminal amino acid at one end of the chain (e.g., amino terminal) therefore can have a free amino group, while the terminal amino acid at the other end of the chain (e.g., carboxy terminal) can have a free carboxyl group. As used herein, the term “amino terminus” (e.g., abbreviated N-terminus) can refer to the free α-amino group on an amino acid at the amino terminal of a peptide or to the α-amino group (e.g., imino group when participating in a peptide bond) of an amino acid at any other location within the peptide. Similarly, the term “carboxy terminus” can refer to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether or thioether as opposed to an amide bond.

The terms “nucleotide,” “oligonucleotide,” “polynucleotide,” “polynucleic acid,” or “nucleic acid” may refer to any molecule comprising nucleic acids, such as short single- or double-stranded DNA or RNA molecules. A nucleotide may comprise deoxyribonucleotides, ribonucleotides, modified deoxyribonucleotides or ribonucleotides, derivatives of deoxyribonucleotides or ribonucleotides, synthetic nucleotides, other nucleotides comprising various nucleobases or various sugars, or combinations thereof. As used herein, “nucleotide,” “oligonucleotide,” “polynucleotide,” “polynucleic acid,” or “nucleic acid” include any single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), oligonucleotide complementary to natural antisense transcripts (NATs) sequences, siRNA, snRNA, aptamer, gapmer, anti-miR, splice blocker ASO, or U1 Adapter. Within the peptide oligonucleotide complexes described herein, “nucleotide,” “oligonucleotide,” “polynucleotide,” “polynucleic acid,” or “nucleic acid” may be intended for modulating gene or protein expression, or for modulating intermolecular pr intramolecular interactions, and may each be considered a target-binding agent capable of binding a target molecule. The target may be a protein, nucleic acid, or other non-nucleic acid molecule. When the target is a nucleic acid, the sequence of a target molecule may be derived from an RNA (e.g., an mRNA or a pre-mRNA) or an open reading frame (ORF) of a gene or protein coding sequence. The sequence of a target molecule may be found in or derived from the coding region or the non-coding region of a gene, or it may be found in or derived from the mature mRNA (e.g., an mRNA which has been spliced, polyadenylated, capped, and exported to the cytosol for translation) or the immature pre-mRNA. The target binding agent may be the complement to such target molecule sequence (e.g., an open reading frame, non-coding sequence, or RNA).

As used herein, the term “complement” or “reverse complement” may refer to a nucleotide sequence that is fully or partially reverse complementary to a target or reference sequence. The term “complementary” may be used interchangeably with “reverse complementary” or “antisense” to describe nucleotide sequences that form base-pairing interactions (e.g., A/T, A/U, or C/G interactions) with a target or reference nucleotide sequence.

As used herein, the term “antisense oligonucleotide” includes small, noncoding, and diffusible molecules, containing about 15-35 nucleotides that form a reverse complement of a nucleic acid target sequence (e.g., a transcript or an mRNA molecule). In some embodiments, the antisense molecule may be fully reverse complementary to the target sequence. In some embodiments, the antisense molecule may comprise one or more base mismatches relative to the target sequence. As used herein, “antisense” may refer to nucleotides of varying chemistries, whether natural (RNA and/or DNA) or synthetic (e.g. 2′ pentose sugar modifications, 2′F, 2′OMe, LNA, PNA, and/or morpholino) with natural or synthetic linkages (e.g. phosphodiester, phosphorothioate, phosphorodiamidate, or thiophosphorodiamidate), as the context requires, and can comprise oligonucleotides, ribonucleotides, ribonucleosides, deoxyribonucleotides, deoxyribonucleosides, may be single stranded or double stranded in whole or in part or in any combination, and any of the forgoing in a modified form and in any combination to form a polynucleic acid. Similarly, thiophosphorodiamidate linkages may be used. Such polynucleic acid can further contain modified bases (e.g., synthetic purines or pyrimidines whose chemistries differ from that of adenine, cytosine, guanine, thymine, or uracil) or contain other atypical elements or chemistries. In various embodiments, antisense RNA containing 19-23 nucleotides (nt), or 15-35 nt, that complement target RNA. Antisense RNAs are about 5 to 30 nt in length, 10 to 25 nt in length, 15 to 25 nt in length, 19 to 23 nt in length, or at least 10 nt in length, at least 15 nt in length, at least 20 nt in length, at least 25 nt in length, or at least 30 nt in length, at least 50 nt in length, at least 100 nucleotides in length. Non-limiting examples of antisense oligonucleotides (ASOs) include aptamers, gapmers, anti-miRs, siRNAs, miRNAs, snRNAs, splice blocker ASOs, and U1 adapters.

As used herein, the term “interfering RNA” or “inhibitory RNA” is used interchangeably and includes RNA molecules that are involved in sequence-specific suppression of gene expression by forming a double-stranded RNA. As used herein, “interfering RNA” or “inhibitory RNA” can comprise ribonucleotides, ribonucleosides, deoxyribonucleotides, deoxyribonucleosides, may be single stranded or double stranded in whole or in part or in any combination, and any of the forgoing in a modified form and in any combination to form a polynucleic acid. Such polynucleic acid can further contain modified bases or contain other atypical elements or chemistries. Common forms of “interfering RNA” or “inhibitory RNA” include small inhibitory RNA (siRNA or RNAi), and dsRNA, ssRNA, hairpin RNA and other known structures. In various embodiments, inhibitory RNAs are about 5 to 30 nt in length, 10 to 25 nt in length, 15 to 25 nt in length, 19 to 23 nt in length, or at least 10 nt in length, at least 15 nt in length, at least 20 nt in length, at least 25 nt in length, or at least 30 nt in length, at least 50 nt in length, at least 100 nucleotides in length.

As used herein, the term “nuclear RNA” includes any RNA molecules that are present in the nucleus of a cell. As used herein, “nuclear RNA” can comprise small nuclear RNA (snRNA), spliceosomal RNA, and other known structures.

As used herein, the term “U1 adaptor” includes bifunctional oligonucleotides with a target domain complementary to a site in the vicinity of the target gene's polyadenylation (polyA) site and a U1 domain that binds to the U1 small nuclear RNA component of the U1 small nuclear ribonucleoprotein (U1 snRNP). U1 Adaptors can be used as synthetic oligonucleotides to recruit endogenous U1 snRNP to a target sequence or site. As used herein, U1 adapters can comprise any nucleotide sequence complementary to the ssRNA component of the U1 small nuclear ribonucleoprotein (U1 snRNP). In various embodiments, U1 adapters are about 5 to 30 nt in length, 10 to 25 nt in length, 15 to 25 nt in length, 19 to 23 nt in length, or at least 10 nt in length, at least 15 nt in length, at least 20 nt in length, at least 25 nt in length, or at least 30 nt in length, at least 50 nt in length, at least 100 nucleotides in length. nucleotides in length and complementary to any sequence along the U1 domain or U1 small nuclear ribonucleoprotein (U1 snRNP) splicing factor.

As used herein, the term “peptide construct” or “peptide complex” can refer to a molecule comprising one or more peptides of the present disclosure that can be conjugated to, linked to, or fused to one or more cargo molecules. In some cases, the cargo molecules are nucleotide target-binding agents. In some cases, cargo molecules are active agents. The term “active agent” can refer to any molecule, e.g., any molecule that is capable of eliciting a biological effect and/or a physical effect (e.g., emission of radiation) which can allow the localization, detection, or visualization of the respective peptide construct.

In various embodiments, the term “active agent” refers to a therapeutic and/or diagnostic agent. A peptide construct of the present disclosure can comprise a TfR-binding peptide that is linked to one or more active agents via one or more linker moieties (e.g., cleavable or stable linker) as described herein. An active agent can be an oligonucleotide (also referred to as a nucleotide or nucleic acid) of a peptide oligonucleotide complex, also referred to as a “target-binding agent” or “target-binding nucleotide.” An active agent can also be, but is not limited to, a small molecule, peptide, or protein.

As used herein, the terms “comprising” and “having” can be used interchangeably. For example, the terms “a peptide comprising an amino acid sequence of SEQ ID NO: 96” and “a peptide having an amino acid sequence of SEQ ID NO: 96” can be used interchangeably.

As used herein, and unless otherwise stated, the term “TfR” or “transferrin receptor” is a class of protein used herein and can refer to a transferrin receptor from any species (e.g., human or murine TfR or any human or non-human animal TfR). In some cases, and as used herein, the term “TfR” or “transferrin receptor” refers to human TfR (hTfR) and can include TfR or any of the known TfR homologs or orthologs, including TfR1, TfR2, soluble TfR, or any combination or fragment (e.g., ectodomain) thereof.

The term “engineered,” when applied to a polynucleotide, denotes that the polynucleotide has been removed from its natural genetic milieu and is thus free of other extraneous or unwanted coding sequences, and is in a form suitable for use within genetically engineered protein production systems. Such engineered molecules are those that are separated from their natural environment and include cDNA and genomic clones (e.g., a prokaryotic or eukaryotic cell with a vector containing a fragment of DNA from a different organism).

Engineered DNA molecules of the present disclosure may be free of other genes with which they are ordinarily associated but may include naturally occurring or non-naturally occurring 5′ and 3′ untranslated regions such as enhancers, promoters and terminators.

An “engineered” polypeptide or protein is a polypeptide or protein that is found in a condition other than its native environment, such as apart from blood and animal tissue. In a preferred form, the engineered polypeptide is substantially free of other polypeptides, particularly other polypeptides of animal origin. It is preferred to provide the polypeptides in a highly purified form, e.g., greater than 90% pure, greater than 92% pure, greater than 95% pure, more preferably greater than 98% pure or greater than 99% pure. When used in this context, the term “engineered” does not exclude the presence of the same polypeptide in alternative physical forms, such as dimers, heterodimers and multimers, or alternatively glycosylated, carboxylated, modified, or derivatized forms.

An “engineered” peptide or protein is a polypeptide that is distinct from a naturally occurring polypeptide structure, sequence, or composition. Engineered peptides include non-naturally occurring, artificial, isolated, synthetic, designed, modified, or recombinantly expressed peptides. Provided herein are engineered TfR-binding peptides, variants, or fragments thereof. These engineered TfR-binding peptides can be further linked to an active agent or a detectable agent. The active agent can be a half-life extending moiety.

Polypeptides of the disclosure include polypeptides that have been modified in any way, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties. For example, single or multiple amino acid substitutions (e.g., conservative amino acid substitutions) are made in the naturally occurring sequence (e.g., in the portion of the polypeptide outside the domain(s) forming intermolecular contacts). A “conservative amino acid substitution” can refer to the substitution in a polypeptide of an amino acid with a functionally similar amino acid. The following six groups each contain amino acids that can be conservative substitutions for one another: i) Alanine (A), Serine (S), and Threonine (T); ii) Aspartic acid (D) and Glutamic acid (E); iii) Asparagine (N) and Glutamine (Q); iv) Arginine (R) and Lysine (K); v) Isoleucine (I), Leucine (L), Methionine (M), and Valine (V); vi) Phenylalanine (F), Tyrosine (Y), and Tryptophan (W).

The terms “polypeptide fragment” and “truncated polypeptide” as used herein can refer to a polypeptide that has an amino-terminal and/or carboxy-terminal deletion as compared to a corresponding full-length peptide or protein. In various embodiments, fragments are at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

In various embodiments, the interface residues of the TfR-binding peptides of the present disclosure (e.g., those amino acid residues that interact with TfR for receptor binding) can be divided between two largely helical domains of the peptide. In some cases, the interface residues can comprise residues corresponding to residues 5-25 (e.g., and comprising corresponding residues G5, A7, S8, M11, N14, L17, E18, and E21), with reference to SEQ ID NO: 96, or corresponding to residues 35-51 (e.g., and comprising corresponding residues L38, L41, L42, L45, D46, H47, H49, S50, and Q51), with reference to SEQ ID NO: 96, or both. For example, the interface residues can comprise residues corresponding to residues 5-25 (e.g., and comprising corresponding residues G5, A7, S8, M11, N14, L17, E18, and E21), with reference to SEQ ID NO: 96, or corresponding to residues 35-51 (e.g., and comprising corresponding residues L38, L41, L42, L45, D46, H47, H49, S50, and Q51), with reference to SEQ ID NO: 96. In some embodiments, a TfR-binding peptide can comprise a fragment of a peptide provided herein, wherein the fragment comprises the minimum interface residues for binding, for example residues corresponding to residues 5-25 (e.g., and comprising corresponding residues G5, A7, S8, M11, N14, L17, E18, and E21), with reference to SEQ ID NO: 96, or corresponding to residues 35-51 (e.g., and comprising corresponding residues L38, L41, L42, L45, D46, H47, H49, S50, and Q51), with reference to SEQ ID NO: 96. In some cases, the TfR-binding peptide is a peptide having the sequence set forth in SEQ ID NO: 96 comprising the TfR-binding residues corresponding to residues G5, A7, S8, M11, N14, L17, E18, and E21 of the domain and corresponding to residues L38, L41, L42, L45, D46, H47, H49, S50, and Q51 of the second domain, with reference to SEQ ID NO: 96.

As used herein, the terms “peptide” or “polypeptide” in conjunction with “variant” “mutant” or “enriched mutant” or “permuted enriched mutant” can refer to a peptide or polypeptide that can comprise an amino acid sequence wherein one or more amino acid residues are inserted into, deleted from and/or substituted into the amino acid sequence relative to another polypeptide sequence. In various embodiments, the number of amino acid residues to be inserted, deleted, or substituted is at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Variants of the present disclosure include peptide conjugates or fusion molecules (e.g., peptide constructs).

A “derivative” of a peptide or polypeptide can be a peptide or polypeptide that can have been chemically modified, e.g., conjugation to another chemical moiety such as, for example, polyethylene glycol, albumin (e.g., human serum albumin), phosphorylation, and glycosylation.

A “derivative” of a nucleoside, nucleotide, oligonucleotide, or polynucleotide can include one or more nucleotide/backbone modifications on the referenced nucleoside oligonucleotide or polynucleotide sequence or to the linkage between nucleotides, or to the end groups, such as the 3′ or 5′ end. The (typically phosphodiester-based) linker, the (typically ribose or deoxyribose) sugar, the (typically hydroxyl) 3′ or 5′ end. The phosphonate, the ribose, or the base may be modified in such derivatives.

The term “% sequence identity” can be used interchangeably herein with the term “% identity” and can refer to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 95%, or at least 99% or more sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The terms “% sequence homology” or “percent sequence homology” or “percent sequence identity” can be used interchangeably herein with the terms “% homology,” “% sequence identity,” or “% identity” and can refer to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 95%, or at least 99% or more sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

A protein, polypeptide, oligonucleotide, or peptide oligonucleotide complex can be “substantially pure,” “substantially homogeneous”, or “substantially purified” when at least about 60% to 75% of a sample exhibits a single species of polypeptide. The polypeptide or protein can be monomeric or multimeric. A substantially pure polypeptide or protein can typically comprise about 50%, 60%, 70%, 80% or 90% W/W of a protein sample, more usually about 95%, and e.g., will be over 99% pure. Protein purity or homogeneity can be indicated by a number of means well known in the art, such as polyacrylamide gel electrophoresis of a protein sample, followed by visualizing a single polypeptide band upon staining the gel with a stain well known in the art. For certain purposes, higher resolution is provided by using high-pressure liquid chromatography (e.g., HPLC) or other high-resolution analytical techniques (e.g., LC-mass spectrometry).

A protein or polypeptide or oligo nucleotide or peptide-oligonucleotide complex may be manufactured to be at least 90% pure, 91% pure, 92% pure, 94% pure, 94% pure, 95% pure, 96% pure, 97% pure, 98% pure, 99% pure, or more than 99% pure.

As used herein, the term “pharmaceutical composition” can generally refer to a composition suitable for pharmaceutical use in a subject such as an animal (e.g., human or mouse). A pharmaceutical composition can comprise a pharmacologically effective amount of an active agent and a pharmaceutically acceptable carrier. The term “pharmacologically effective amount” can refer to that amount of an agent effective to produce the intended biological or pharmacological result.

As used herein, the term “pharmaceutically acceptable carrier” can refer to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. A “pharmaceutically acceptable carrier” can include a solution buffered by phosphate, histidine, citrate, or other buffers or made isoosmotic by sodium chloride, dextrose, mannitol, trehalose, or other components. Suitable pharmaceutical carriers and formulations are described in Remington's Pharmaceutical Sciences, 21st Ed. 2005, Mack Publishing Co, Easton. A “pharmaceutically acceptable salt” can be a salt that can be formulated into a compound for pharmaceutical use including, e.g., metal salts (sodium, potassium, magnesium, calcium, etc.) and salts of ammonia or organic amines.

As used herein, the terms “treat”, “treating” and “treatment” can refer to a method of alleviating or abrogating a biological disorder and/or at least one of its attendant symptoms. As used herein, to “alleviate” a disease, disorder or condition, for example, means reducing the severity and/or occurrence frequency of the symptoms of the disease, disorder, or condition. Further, references herein to “treatment” can include references to curative, palliative, and prophylactic or diagnostic treatment.

Generally, a cell of the present disclosure can be a eukaryotic cell or a prokaryotic cell. A cell can be an epithelial cell. A cell can be an animal cell or a plant cell. An animal cell can include a cell from a marine invertebrate, fish, insects, amphibian, reptile, or mammal. A mammalian cell can be obtained from a primate, ape, equine, bovine, porcine, canine, feline, or rodent. A mammal can be a primate, ape, dog, cat, rabbit, ferret, or the like. A rodent can be a mouse, rat, hamster, gerbil, hamster, chinchilla, or guinea pig. A bird cell can be from a canary, parakeet or parrots. A reptile cell can be from a turtles, lizard or snake. A fish cell can be from a tropical fish. For example, the fish cell can be from a zebrafish (e.g., Danino rerio). A worm cell can be from a nematode (e.g., C. elegans). An amphibian cell can be from a frog. An arthropod cell can be from a tarantula or hermit crab.

A mammalian cell can also include cells obtained from a primate (e.g., a human or a non-human primate). A mammalian cell can include a blood cell, a stem cell, an epithelial cell, connective tissue cell, hormone secreting cell, a nerve cell, a skeletal muscle cell, a cardiovascular muscle cell, or an immune system cell. In preferred embodiments, the methods and compositions of the present disclosure are used in combination with one or more mammalian cells.

As used herein, the term “vector,” generally refers to a DNA molecule capable of replication in a host cell and/or to which another DNA segment can be operatively linked so as to bring about replication of the attached segment. A plasmid is an exemplary vector.

As used herein, the term “subject,” generally refers to a human or to another animal. A subject can be of any age, for example, a subject can be prenatal, an infant, a toddler, a child, a pre-adolescent, an adolescent, an adult, or an elderly individual.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are in relation to the other endpoint, and independently of the other endpoint. The term “about” as used herein refers to a range that is 15% plus or minus from a stated numerical value within the context of the particular usage. For example, about 10 can include a range from 8.5 to 11.5.

### Peptides

Disclosed herein are peptide sequences, such as those listed in TABLE 1, that may form the peptide portion of a peptide oligonucleotide complex. The peptides may be capable of binding to TfR or any of the known TfR homologs, including TfR1, TfR2, soluble TfR, or any combination or fragment (e.g., ectodomain) thereof. A peptide capable of binding a transferrin receptor or a TfR homolog may be referred to herein as a transferrin receptor-binding peptide or a TfR-binding peptide. In some embodiments, peptides disclosed herein can penetrate, cross, or enter target cells in a TfR-mediated manner. These cell layers or cells can include TfR-expressing endothelial cells, epithelial cells, and TfR-expressing cells of various tissues or organs such as tumor cells, brain cells, cancerous or tumor cells, liver cells, pancreas cells, colon cells, ovarian cells, breast cells, and/or lung cells, or any combination thereof. As disclosed herein peptide sequences, variants, and properties of the peptides that form the TfR-binding peptide portion of the peptide oligonucleotide complex may be referred to as TfR-binding peptides of the present disclosure, or peptides of the present disclosure. It understood that such peptides are described in the context of the peptide oligonucleotide complexes disclosed, such as a peptide or TfR-binding peptide within the peptide oligonucleotide complex, with the accorded alterations, functions and uses described.

The peptide oligonucleotide complexes of the present disclosure include TfR-binding peptides and peptide variants within the peptide oligonucleotide complex that enable TfR-mediated transport across cellular layers as described herein. In various embodiments, the present disclosure provides methods and compositions that enable TfR-mediated transport across cellular layers (e.g., endothelial cells or epithelial cells) or cell membranes. In addition to the BBB, various other cells, tissues, and organs express TfR. Single cells expressing TfR can include hepatocytes, erythrocytes and erythrocyte precursors in bone marrow, immune cells, stem cells, and rapidly dividing cells. Tissues and organs expressing TfR can include the brain (e.g., cerebral cortex, hippocampus, caudate, cerebellum), endocrine tissues (e.g., thyroid, parathyroid, and adrenal glands), bone marrow and immune system (e.g., appendix, lymph node, tonsil, spleen), muscle tissues (e.g., heart, skeletal, and smooth muscle), liver, gallbladder, pancreas, gastrointestinal tract (e.g., oral mucosa, esophagus, stomach, duodenum, small intestine, colon, rectum), kidney, urinary bladder, female tissues (e.g., fallopian tube, breast, vagina, cervix, endometrium, ovary, and placenta), adipose and soft tissue, and skin. Thus, the TfR-binding peptides of the present disclosure can be used to target these cells, tissues, and organs and deliver an active agent to these cells, tissues, and organs via, for example, TfR-mediated transcytosis (e.g., across cellular barrier such as the BBB) or TfR-mediated endocytosis (e.g., across cell membranes into cells) or TfR-mediated accumulation in tissues to treat and/or prevent a disease or condition in one or more of these cells, tissues, or organs.

In various embodiments, the present disclosure provides methods and compositions that enable TfR-mediated transport and delivery to cancer cells expressing TfR. Cancers overexpressing TfR can include ovarian cancer, colon cancer, lung cancer, cancer located in the bone or bone marrow, glioblastoma, astrocytoma, glioma, medulloblastoma, ependymoma, choroid plexus carcinoma, midline glioma, diffuse intrinsic pontine glioma (DIPG), breast cancer, liver cancer, colon cancer, brain cancer, spleen cancer, cancers of the salivary gland, kidney cancer, muscle cancers, bone marrow cell cancers, skin cancer, genitourinary cancer, osteosarcoma, muscle-derived sarcoma, melanoma, head and neck cancer, neuroblastoma, prostate cancer, bladder cancer, acute lymphocytic leukemia, acute myeloid leukemia, chronic lymphocytic leukemia, chronic myeloid leukemia, Hodgkin lymphoma, Non-Hodgkin lymphoma, or a CMYC-overexpressing cancer. Cancer cells may be from a primary cancer or from cancer metastases.

TfR-binding peptide oligonucleotide complexes that can be used to prevent and/or treat a cancer include those comprising a TfR binding peptide and an active agent with anti-tumor activity such as a fused IL15/IL15Ra complex, IFNgamma, and anti-CD3 agents. Examples of such peptide constructs (e.g., fusion peptides) are those having an amino acid sequence set forth in any one of SEQ ID NO: 135-SEQ ID NO: 140. In some embodiments, TfR-binding peptide constructs are fused to IL-15 to recruit and stimulated the immune T cells or NK cells. In some embodiments, TfR-binding peptide constructs are fused to INFg to activate macrophages and upregulated MHC in tumor cells. In some embodiments, TfR-binding peptide constructs are fused to CD3 to bind T cells in addition to binding TfR on cancer cells to create an immune synapse and activate T cells to target the cancer.

Generally, the TfR-binding peptides of the present disclosure can be used in combination with various classes of active agent. The TfR-binding peptides of the present disclosure can be conjugated to, linked to, or fused to one or more of those active agents. In various aspects, an active agent is an immunotherapeutic agent, a CTLA-4 targeting agent, a PD-1 targeting agent, a PDL-1 targeting agent, an IL15 agent, a fused IL15/IL15Ra complex agent, an IFNgamma agent, an anti-CD3 agent, an ion channel modulator, a Kv1.3 inhibitor, an auristatin, MMAE, a maytansinoid, DM1, DM4, doxorubicin, a calicheamicin, a platinum compound, cisplatin, a taxane, paclitaxel, SN-38, a BACE inhibitor, a Bcl-xL inhibitor, WEHI-539, venetoclax, ABT-199, navitoclax, AT-101, obatoclax, a pyrrolobenzodiazepine or pyrrolobenzodiazepine dimer, a dolastatin, or a neurotransmitter such as neurotensin.

In some embodiments, the peptides as discloses herein can cross cellular layers or barriers (e.g., BBB) or cell membranes via, for example, TfR-mediated vesicular transcytosis and TfR-mediated endocytosis, respectively. In addition to binding TfR and promote transcytosis and/or endocytosis, the peptides of the present disclosure can also bind to additional target proteins on cells such as cancer cells. In some cases, a peptide is a peptide oligonucleotide complex comprising a TfR-binding peptide conjugated to, linked to, or fused to a nucleotide target-binding agent. In some case, a peptide oligonucleotide complex may further comprise a targeting moiety or an active agent (e.g., a therapeutic or diagnostic agent) such as a small molecule or a peptide that has an affinity for an additional target protein (e.g., receptor or enzyme) or the nucleotide of the peptide oligonucleotide complex. In some cases, the TfR-binding peptide is linked to a cargo molecule and enables or promotes TfR-mediated transcytosis of the cargo molecule across the BBB or TfR-mediated endocytosis into a cell. In some instances, and subsequent to transcytosis, a peptide construct comprising the TfR-binding peptide and a cargo moiety can target a specific cell or tissue in the CNS and exert a biological effect (e.g., binding a target protein or binding a target nucleic acid sequence) upon reaching said cell or tissue. In some cases, a peptide construct of the present disclosure exerts a biological effect that is mediated by the TfR-binding peptide, the cargo molecule or active agent, or a combination thereof. In some cases, a TfR-binding peptide construct of the present disclosure comprising one or more active agents (e.g., therapeutic agents) can transport and/or deliver the one or more active agents into cells that express TfR. In some cases, the TfR-binding peptide accumulates in tissues in the CNS. In some cases, off-target effects are reduced due to CNS-specific accumulation. In some cases, the TfR-binding peptide accumulates in tissue outside of the CNS (e.g., liver, kidney, spleen, or skin). In some cases, the cells expressing TfR are tumor cells and the TfR-binding peptide construct delivers anti-tumor agents to these tumor cells. In some cases, the anti-tumor agents alone show no or only very limited therapeutic efficacy against the tumor cells; however, when the anti-tumor agents are combined with the TfR-binding peptides of the present disclosure as, for example, a peptide construct, the therapeutic efficacy of these anti-tumor agents is significantly improved.

The peptide oligonucleotide complexes of the present disclosure include TfR-binding peptide and peptide variants within the peptide oligonucleotide complex that enable TfR-mediated transport across cellular layers as described herein for use in treating pain, cancer and neurological and immunological disorder, amongst other diseases described. In some embodiments, the TfR-binding peptide oligonucleotide complexes of the present disclosure can induce a biologically relevant response. In some embodiments, the biologically relevant response can be induced after intravenous dose, and in some embodiments, after a single intravenous dose. In some embodiments, the TfR-binding peptides can be used in combination with various other classes of therapeutic compounds used to treat and/or prevent pain, neuropathic pain or other neurological disorders such as neurodegenerative disorders, infectious diseases, immunological disorders (e.g., autoimmune diseases). Binding of the herein described peptides and peptide constructs and peptide complexes (e.g., peptide conjugates, fusion peptides, or recombinantly produced peptide constructs) to TfR and subsequent transport across a cell layer or barrier such as the BBB (e.g., via TfR-mediated vesicular transcytosis) or a cell membrane (e.g., via TfR-mediated endocytosis) can have implications in a number of diseases, conditions, or disorders associated with chronic pain (e.g., headaches or migraine), neuropathic pain, obesity, insulin resistance, opioid addiction, or other neurologic or psychiatric disorders in a subject (e.g., a human).

Binding of the herein described peptides and peptide constructs and peptide complexes (e.g., peptide conjugates, fusion peptides, or recombinantly produced peptide constructs) to TfR and subsequent transport across a cell layer or barrier such as the BBB (e.g., via vesicular transcytosis) or a cell membrane (e.g., via endocytosis) can have implications in a number of diseases, conditions, or disorders associated with neurodegeneration. Neurodegenerative diseases that can treated, prevented, or diagnosed with the herein described TfR-binding peptides can include Alzheimer's disease, Amyotrophic lateral sclerosis, Friedreich's ataxia, Huntington's disease, Lewy body disease, Parkinson's disease, Spinal muscular atrophy, Motor neuron disease, Lyme disease, Ataxia-telangiectasia, Autosomal dominant cerebellar ataxia, Batten disease, Corticobasal syndrome, Creutzfeldt-Jakob disease, Fragile X-associated tremor/ataxia syndrome, Kufor-Rakeb syndrome, Machado-Joseph disease, multiple sclerosis, chronic traumatic encephalopathy, or frontotemporal dementia.

Binding of the herein described peptides, peptide constructs, and peptide complexes (e.g., peptide conjugates, fusion peptides, or recombinantly produced peptide constructs) to TfR and subsequent transport across a cell layer or barrier such as the BBB (e.g., via vesicular transcytosis) or a cell membrane (e.g., via endocytosis) can have implications in a number of autoimmune diseases to therapeutically address auto-immune or inflammatory disorders in the brain. In some case, an autoimmune disease can be treated and/or prevented by conjugating, linking, or fusing the TfR-binding peptide to an active agent that can act on a nucleotide sequence encoding an ion channel modulator such as Kv1.3 potassium channel inhibitor. Additional diseases that can be treated and/or prevented using ion channel modulator such as Kv1.3 potassium channel inhibitors can include psoriasis and other non-brain autoimmune diseases due to its effect on effector T cells, and for neuroinflammatory and neurodegenerative diseases, for example, multiple sclerosis, Alzheimer's, Parkinson's, traumatic brain injury, or radiation therapy toxicity. In some cases, Kv1.3 potassium channel inhibitors can be an oligonucleotide gene modulator, a small molecule (e.g., domatinostat tosylate), or a peptide (e.g., Vm24 or an ShK peptide, such as ShK-170, ShK-186, or ShK-192, or any fragments or derivatives thereof) or any combinations thereof. (See e.g., Bartok et al. An engineered scorpion toxin analogue with improved Kv1.3 selectivity displays reduced conformational flexibility, Sci Rep. 2015; 5: 18397).

In some embodiments, the TfR-binding peptides of the present disclosure can be used for the treatment and prevention of various neurological diseases including but not limited to epilepsy, schizophrenia, depression, anxiety, bipolar disorder, developmental brain disorders (e.g., autism spectrum), or mood disorder.

In some embodiments, the TfR-binding peptides of the present disclosure can be used for the treatment and prevention of Crohn's disease or, more generally, inflammatory bowel diseases. In some cases, the TfR-binding peptides of the present disclosure show high uptake and retention in glandular cells of the intestine, which can express high amounts of TfR.

In various embodiments, the therapeutic efficacy of these drugs can be significantly improved when used in combination with the TfR-binding peptides of the present disclosure compared to administration without conjugation to the TfR-binding peptides as described herein. In various embodiments, the efficacy can be improved due to higher delivery and achieved levels of drug in the tissue or cell of interest. In various embodiments, the efficacy can be improved by increasing the relative level of the drug in the tissue or cell or interest and reducing the level of the drug in other tissues or compartments, thereby improving the therapeutic window or reducing toxic side effects.

The peptide oligonucleotide complexes of the present disclosure include TfR binding peptide and peptide variants within the peptide oligonucleotide complex that enable competition on TfR with endogenous molecules. In other embodiments, peptides as described herein compete with endogenous molecules for binding to TfR. As described herein, “compete” or peptide competition for binding to target protein such as TfR encompasses, but is not limited to, steric hindrance, occupying binding sites of the target protein, non-covalent interactions, such as salt bridges or hydrophobic interactions, crosslinking, covalent interactions, sequestration, allosteric modulation, or any combination thereof.

In some embodiments, peptides of the present disclosure can bind to any of the known TfR homologs, including TfR1, TfR2, soluble TfR, or any combination or fragment (e.g., ectodomain) thereof. Thus, as used herein, “TfR” can refer to any known homolog, derivative, fragment, or member of the TfR family including TfR1, TfR2, and a soluble TfR. In other embodiments, peptides are capable of binding to one, one or more, or all TfR homologs. In some embodiments, peptides of the present disclosure can bind to a TfR and promote a particular biological effect such as vesicular transcytosis. In some embodiments, peptides of the present disclosure, including peptides, peptide complexes, and peptide constructs with amino acid sequences set forth in SEQ ID NO: 1-SEQ ID NO: 134 and SEQ ID NO: 306-SEQ ID NO: 335, and any derivatives or variant thereof, prevent or decrease the binding of endogenous TfR binders (e.g., transferrin or any derivatives such as apo-transferrin or holo-transferrin) to TfR. In some embodiments, peptides of the present disclosure comprise derivatives and variants with at least 70% homology, at least 75% homology, at least 80% homology, at least 85% homology, at least 90% homology, at least 95% homology, at least 96% homology, at least 97% homology, at least 98% homology, or at least 99% homology or at least 100% homology to amino acid sequences set forth in SEQ ID NO: 1-SEQ ID NO: 134.

In some embodiments, peptides bind to TfR with equal, similar, or greater affinity (e.g., lower dissociation constant K_{D}) as compared to endogenous molecules (e.g., transferrin, holotransferrin (iron-bound transferrin), apotransferrin (transferrin not bound to iron), or any other endogenous TfR ligands) or other exogenous molecules. In some embodiments, the peptide can have a K_{D} of less than 50 μM, less than 5 μM, less than 500 nM, less than 100 nM, less than 40 nM, less than 30 nM, less than 20 nM, less than 10 nM, less than 1 nM, or less than 0.1 nM. In some embodiments, peptide transport by TfR is improved by having a lower affinity (e.g., a higher dissociation constant K_{D}) as compared to endogenous molecules. In some embodiments, peptide transport by TfR is improved by having a faster off rate or higher k_{off} than endogenous molecules. In some embodiments, the off rate or k_{off} is similar to that of transferrin. In some embodiments, peptide transport is improved by having a faster on rate or a higher kₒₙ, optionally such as higher than that of transferrin. In other embodiments, one or more conserved residues at the transferrin (Tf)-TfR-binding interface are also present in the amino acid sequences of the peptides described herein.

In some embodiments, peptides that exhibit an improved TfR receptor binding show improved transcytosis function. In some embodiments, peptides that exhibit an improved TfR receptor binding show no or small changes in transcytosis function. In some embodiments, peptides that exhibit an improved TfR receptor binding show reduced transcytosis function. In some embodiments, the peptide binds at a site of high homology between human and murine TfR, including one or more, or all, of the amino acid domains corresponding to residues 506-510, 523-531, and 611-662 of the human TfR (SEQ ID NO: 349, MMDQARSAFSNLFGGEPLSYTRFSL ARQVDGDNSHVEMKLAVDEEENADNNTKANVT KPK). In some embodiments, the regions of TfR to which the peptides disclosed herein or variants thereof bind all or in part to such TfR domains. In some embodiments, the peptides disclosed herein bind to any one, any two, or all three of the TfR regions of high homology including the amino acid domains corresponding to residues 506-510, 523-531, and 611-662 of the human TfR (SEQ ID NO: 349). In some embodiments the peptides disclosed herein bind at least to the domain corresponding to residues 611-662 of the human TfR.

The peptide oligonucleotide complexes of the present disclosure include TfR binding peptide and peptide variants within the peptide oligonucleotide complex that enable the TfR-binding peptide portion to be modulated and optimized. In some embodiments, the association constant (kₐ) and dissociation constant (k_{d})values of a TfR-binding peptide can be modulated and optimized (e.g., via amino acid substitutions) to provide an optimal ratio of TfR-binding affinity and efficient transcytosis function.

In some embodiments, peptides disclosed herein or variants thereof bind to TfR at residues found in the binding interface (e.g., the binding domain or the binding pocket) of TfR with other exogenous or endogenous ligands (e.g., transferrin (Tf), Tf derivatives, or Tf-like peptides or proteins). In some embodiments, a peptide disclosed herein or a variant thereof, which binds to TfR, comprises at least 70% homology, at least 75% homology, at least 80% homology, at least 85% homology, at least 90% homology, at least 95% homology, at least 96% homology, at least 97% homology, at least 98% homology, or at least 99% homology or at least 100% homology to a sequence that binds residues of TfR, which makeup the binding pocket. In some embodiments, a peptide disclosed herein or a variant thereof, which binds to TfR, comprises at least 70% homology, at least 75% homology, at least 80% homology, at least 85% homology, at least 90% homology, at least 95% homology, at least 96% homology, at least 97% homology, at least 98% homology, or at least 99% homology or at least 100% homology to an endogenous or exogenous polypeptide known to bind TfR, for example, endogenous Transferrin or any one of the peptides listed in TABLE 1. In other embodiments, a peptide described herein binds to a protein of interest, which comprises at least 70% homology, at least 75% homology, at least 80% homology, at least 85% homology, at least 90% homology, at least 95% homology, at least 96% homology, at least 97% homology, at least 98% homology, or at least 99% homology or at least 100% homology to TfR, a fragment, homolog, or a variant thereof.

In some embodiments, peptides disclosed herein or variants thereof bind regions of TfR that comprise the amino acid residues corresponding to residues 506-510, 523-531, and 611-662 (the numbering of these amino acid residues is based on the following Uniprot reference protein sequence of endogenous human TFRC UniProtKB—P02786 (SEQ ID NO: 349, TFR1_HUMAN)). In some embodiments, the regions of TfR to which the peptides disclosed herein or variants thereof bind overlap with those of Tf, a fragment, homolog, or a variant thereof.

The peptide oligonucleotide complexes of the present disclosure include TfR-binding peptide and peptide variants within the peptide oligonucleotide complex wherein the TfR-binding peptide portion contains conserved TfR-binding motifs as described. In other embodiments, a nucleic acid, vector, plasmid, or donor DNA comprises a sequence that encodes a peptide, peptide construct, or variant or functional fragment thereof, as described in the present disclosure. In further embodiments, certain parts or fragments of TfR-binding motifs (e.g., conserved binding motifs) can be grafted onto a peptide with a sequence of any one of SEQ ID NO: 1-SEQ ID NO: 134 or SEQ ID NO: 306-SEQ ID NO: 335.

In some embodiments, peptides inhibit binding between TfR and Tf, or between TfR and any other protein. In some embodiments, peptides prevent TfR from protein-protein interaction and/or prevent TfR localization to a cell's nucleus. In some cases, peptides deactivate TfR. In some embodiments, peptides can cause TfR to be degraded, or prevent TfR from localization to a cell's nucleus, or prevent TfR from interacting with Tf or Tf-like proteins.

In some embodiments, peptides competitively bind to TfR as compared to endogenous Tf or any other endogenous or exogenous TfR binder by binding to a certain amino acid residue or motif of amino acid residues in TfR. Furthermore, a peptide can be selected for further testing or use based upon its ability to bind to the certain amino acid residue or motif of amino acid residues. The certain amino acid residue or motif of amino acid residues in TfR can be identified an amino acid residue or sequence of amino acid residues that are involved in the binding of TfR to Tf. A certain amino acid residue or motif of amino acid residues can be identified from a crystal structure of the TfR:Tf complex. In some embodiments, peptides (e.g., CDPs) demonstrate the resistance to heat, protease (pepsin), and reduction.

The peptide constructs and peptide complexes (e.g., peptide conjugates or fusion peptides) comprising one or more of the amino acid sequences set forth in SEQ ID NO: 1-SEQ ID NO: 134 or SEQ ID NO: 306-SEQ ID NO: 335 can bind to a protein of interest. In some embodiments, the protein of interest is a TfR. In some embodiments, the peptide constructs and peptide complexes (e.g., peptide conjugates or fusion peptides) that bind to a TfR comprise at least one of the amino acid sequences set forth in SEQ ID NO: 1-SEQ ID NO: 134 or SEQ ID NO: 306-SEQ ID NO: 335. In some embodiments, peptides, peptide constructs, and peptide complexes (e.g., peptide conjugates and fusion molecules) of the present disclosure that bind to a TfR comprise peptide derivatives or variants having at least 70% homology, at least 75% homology, at least 80% homology, at least 85% homology, at least 90% homology, at least 95% homology, at least 96% homology, at least 97% homology, at least 98% homology, or at least 99% homology or at least 100% homology to amino acid sequences set forth in SEQ ID NO: 1-SEQ ID NO: 134.

In some embodiments, a peptide or a library of peptides is designed in silico without derivation from a naturally occurring scaffold of a knotted peptide. In other embodiments, a peptide or a library of peptides is designed in silico by derivation, grafting relevant protein-binding residues, or conserved residues in the protein-binding interface a naturally occurring peptide or protein known to bind to a protein or receptor of interest. In some embodiments, the peptide (e.g., SEQ ID NO: 1-SEQ ID NO: 134 or SEQ ID NO: 306-SEQ ID NO: 335) is a simple helix-turn-helix. In some embodiments, the helix-turn-helix can be used for pharmacophore transfer onto other scaffolds, for example engraftment of the required TfR-engaging surface onto the helix-turn-helix scaffold using fusion tagging.

In some embodiments, a peptide comprising SEQ ID NO: 65 is used as a scaffold or base sequence for further modifications, including addition, deletion, or amino acid substitution. In some embodiments, short sequences of amino acid residues such as GS are added at the N-terminus of a peptide. In some embodiments, peptides lack GS at the N-terminus. In some instances, peptides undergo one or more post-translational modifications.

TABLE 1 lists exemplary peptide sequences according to the methods and compositions of the present disclosure.

TABLE 1
Exemplary Peptide Sequences

| SEQ ID NO | Amino Acid Sequence |
|---|---|
| SEQ ID NO: 1 | REGCASRCTKYNAELEKCEARVSSMSNTEETCVQELFDLLHCVDHCVSQ |
| SEQ ID NO: 2 | REGCASRCTKYNAELEKCEARVMSMSNTEEDCEQELEDLLHCLDHCHSQ |
| SEQ ID NO: 3 | REGCASRCTKYNAELEKCEARVVSMSNTEEDCEQELEDLLHCLDHCHSQ |
| SEQ ID NO: 4 | REGCASRCTKYNAELEKCEARVMSMSNTEETCEQELEDLLHCLDHCHSQ |
| SEQ ID NO: 5 | REGCASRCTKYNAELEKCEARVMSMSNTEEDCVQELEDLLHCLDHCHSQ |
| SEQ ID NO: 6 | REGCASRCTKYNAELEKCEARVMSMSNTEEDCEQELFDLLHCLDHCHSQ |
| SEQ ID NO: 7 | REGCASRCTKYNAELEKCEARVMSMSNTEEDCEQELEDLLHCVDHCHSQ |
| SEQ ID NO: 8 | REGCASRCTKYNAELEKCEARVMSMSNTEEDCEQELEDLLHCLDHCVSQ |
| SEQ ID NO: 9 | REGCASRCTKYNAELEKCEARVVSMSNTEETCEQELEDLLHCLDHCHSQ |
| SEQ ID NO: 10 | REGCASRCTKYNAELEKCEARVVSMSNTEEDCVQELEDLLHCLDHCHSQ |
| SEQ ID NO: 11 | REGCASRCTKYNAELEKCEARVVSMSNTEEDCEQELFDLLHCLDHCHSQ |
| SEQ ID NO: 12 | REGCASRCTKYNAELEKCEARVVSMSNTEEDCEQELEDLLHCVDHCHSQ |
| SEQ ID NO: 13 | REGCASRCTKYNAELEKCEARVVSMSNTEEDCEQELEDLLHCLDHCVSQ |
| SEQ ID NO: 14 | REGCASRCTKYNAELEKCEARVMSMSNTEETCVQELEDLLHCLDHCHSQ |
| SEQ ID NO: 15 | REGCASRCTKYNAELEKCEARVMSMSNTEETCEQELFDLLHCLDHCHSQ |
| SEQ ID NO: 16 | REGCASRCTKYNAELEKCEARVMSMSNTEETCEQELEDLLHCVDHCHSQ |
| SEQ ID NO: 17 | REGCASRCTKYNAELEKCEARVMSMSNTEETCEQELEDLLHCLDHCVSQ |
| SEQ ID NO: 18 | REGCASRCTKYNAELEKCEARVMSMSNTEEDCVQELFDLLHCLDHCHSQ |
| SEQ ID NO: 19 | REGCASRCTKYNAELEKCEARVMSMSNTEEDCVQELEDLLHCVDHCHSQ |
| SEQ ID NO: 20 | REGCASRCTKYNAELEKCEARVMSMSNTEEDCVQELEDLLHCLDHCVSQ |
| SEQ ID NO: 21 | REGCASRCTKYNAELEKCEARVMSMSNTEEDCEQELFDLLHCVDHCHSQ |
| SEQ ID NO: 22 | REGCASRCTKYNAELEKCEARVMSMSNTEEDCEQELFDLLHCLDHCVSQ |
| SEQ ID NO: 23 | REGCASRCTKYNAELEKCEARVMSMSNTEEDCEQELEDLLHCVDHCVSQ |
| SEQ ID NO: 24 | REGCASRCMKYNAELEKCEARVMSMSNTEEDCEQELEDLLHCLDHCHSQ |
| SEQ ID NO: 25 | REGCASRCTKYNDELEKCEARVMSMSNTEEDCEQELEDLLHCLDHCHSQ |
| SEQ ID NO: 26 | REGCASRCTKYNAELEKCEARMMSMSNTEEDCEQELEDLLHCLDHCHSQ |
| SEQ ID NO: 27 | REGCASRCTKYNAELEKCEARVMSMSNTEEDCEQELEDLLYCLDHCHSQ |
| SEQ ID NO: 28 | REGCASRCMKYNDELEKCEARMMSMSNTEEDCEQELEDLLHCLDHCHSQ |
| SEQ ID NO: 29 | REGCASRCMKYNDELEKCEARVMSMSNTEEDCEQELEDLLYCLDHCHSQ |
| SEQ ID NO: 30 | REGCASRCMKYNAELEKCEARMMSMSNTEEDCEQELEDLLYCLDHCHSQ |
| SEQ ID NO: 31 | REGCASRCTKYNDELEKCEARMMSMSNTEEDCEQELEDLLYCLDHCHSQ |
| SEQ ID NO: 32 | REGCASRCMKYNDELEKCEARMMSMSNTEEDCEQELEDLLYCLDHCHSQ |
| SEQ ID NO: 33 | REGCASRCTRYNAELERCEARVSSMSNTEETCVQELFDLLHCVDHCVSQ |
| SEQ ID NO: 34 | REGCASRCTRYNAELERCEARVMSMSNTEEDCEQELEDLLHCLDHCHSQ |
| SEQ ID NO: 35 | REGCASRCTRYNAELERCEARVVSMSNTEEDCEQELEDLLHCLDHCHSQ |
| SEQ ID NO: 36 | REGCASRCTRYNAELERCEARVMSMSNTEETCEQELEDLLHCLDHCHSQ |
| SEQ ID NO: 37 | REGCASRCTRYNAELERCEARVMSMSNTEEDCVQELEDLLHCLDHCHSQ |
| SEQ ID NO: 38 | REGCASRCTRYNAELERCEARVMSMSNTEEDCEQELFDLLHCLDHCHSQ |
| SEQ ID NO: 39 | REGCASRCTRYNAELERCEARVMSMSNTEEDCEQELEDLLHCVDHCHSQ |
| SEQ ID NO: 40 | REGCASRCTRYNAELERCEARVMSMSNTEEDCEQELEDLLHCLDHCVSQ |
| SEQ ID NO: 41 | REGCASRCTRYNAELERCEARVVSMSNTEETCEQELEDLLHCLDHCHSQ |
| SEQ ID NO: 42 | REGCASRCTRYNAELERCEARVVSMSNTEEDCVQELEDLLHCLDHCHSQ |
| SEQ ID NO: 43 | REGCASRCTRYNAELERCEARVVSMSNTEEDCEQELFDLLHCLDHCHSQ |
| SEQ ID NO: 44 | REGCASRCTRYNAELERCEARVVSMSNTEEDCEQELEDLLHCVDHCHSQ |
| SEQ ID NO: 45 | REGCASRCTRYNAELERCEARVVSMSNTEEDCEQELEDLLHCLDHCVSQ |
| SEQ ID NO: 46 | REGCASRCTRYNAELERCEARVMSMSNTEETCVQELEDLLHCLDHCHSQ |
| SEQ ID NO: 47 | REGCASRCTRYNAELERCEARVMSMSNTEETCEQELFDLLHCLDHCHSQ |
| SEQ ID NO: 48 | REGCASRCTRYNAELERCEARVMSMSNTEETCEQELEDLLHCVDHCHSQ |
| SEQ ID NO: 49 | REGCASRCTRYNAELERCEARVMSMSNTEETCEQELEDLLHCLDHCVSQ |
| SEQ ID NO: 50 | REGCASRCTRYNAELERCEARVMSMSNTEEDCVQELFDLLHCLDHCHSQ |
| SEQ ID NO: 51 | REGCASRCTRYNAELERCEARVMSMSNTEEDCVQELEDLLHCVDHCHSQ |
| SEQ ID NO: 52 | REGCASRCTRYNAELERCEARVMSMSNTEEDCVQELEDLLHCLDHCVSQ |
| SEQ ID NO: 53 | REGCASRCTRYNAELERCEARVMSMSNTEEDCEQELFDLLHCVDHCHSQ |
| SEQ ID NO: 54 | REGCASRCTRYNAELERCEARVMSMSNTEEDCEQELFDLLHCLDHCVSQ |
| SEQ ID NO: 55 | REGCASRCTRYNAELERCEARVMSMSNTEEDCEQELEDLLHCVDHCVSQ |
| SEQ ID NO: 56 | REGCASRCMRYNAELERCEARVMSMSNTEEDCEQELEDLLHCLDHCHSQ |
| SEQ ID NO: 57 | REGCASRCTRYNDELERCEARVMSMSNTEEDCEQELEDLLHCLDHCHSQ |
| SEQ ID NO: 58 | REGCASRCTRYNAELERCEARMMSMSNTEEDCEQELEDLLHCLDHCHSQ |
| SEQ ID NO: 59 | REGCASRCTRYNAELERCEARVMSMSNTEEDCEQELEDLLYCLDHCHSQ |
| SEQ ID NO: 60 | REGCASRCMRYNDELERCEARMMSMSNTEEDCEQELEDLLHCLDHCHSQ |
| SEQ ID NO: 61 | REGCASRCMRYNDELERCEARVMSMSNTEEDCEQELEDLLYCLDHCHSQ |
| SEQ ID NO: 62 | REGCASRCMRYNAELERCEARMMSMSNTEEDCEQELEDLLYCLDHCHSQ |
| SEQ ID NO: 63 | REGCASRCTRYNDELERCEARMMSMSNTEEDCEQELEDLLYCLDHCHSQ |
| SEQ ID NO: 64 | REGCASRCMRYNDELERCEARMMSMSNTEEDCEQELEDLLYCLDHCHSQ |
| SEQ ID NO: 65 | GSREGCASRCTKYNAELEKCEARVSSMSNTEETCVQELFDLLHCVDHCVSQ |
| SEQ ID NO: 66 | GSREGCASRCTKYNAELEKCEARVMSMSNTEEDCEQELEDLLHCLDHCHSQ |
| SEQ ID NO: 67 | GSREGCASRCTKYNAELEKCEARVVSMSNTEEDCEQELEDLLHCLDHCHSQ |
| SEQ ID NO: 68 | GSREGCASRCTKYNAELEKCEARVMSMSNTEETCEQELEDLLHCLDHCHSQ |
| SEQ ID NO: 69 | GSREGCASRCTKYNAELEKCEARVMSMSNTEEDCVQELEDLLHCLDHCHSQ |
| SEQ ID NO: 70 | GSREGCASRCTKYNAELEKCEARVMSMSNTEEDCEQELFDLLHCLDHCHSQ |
| SEQ ID NO: 71 | GSREGCASRCTKYNAELEKCEARVMSMSNTEEDCEQELEDLLHCVDHCHSQ |
| SEQ ID NO: 72 | GSREGCASRCTKYNAELEKCEARVMSMSNTEEDCEQELEDLLHCLDHCVSQ |
| SEQ ID NO: 73 | GSREGCASRCTKYNAELEKCEARVVSMSNTEETCEQELEDLLHCLDHCHSQ |
| SEQ ID NO: 74 | GSREGCASRCTKYNAELEKCEARVVSMSNTEEDCVQELEDLLHCLDHCHSQ |
| SEQ ID NO: 75 | GSREGCASRCTKYNAELEKCEARVVSMSNTEEDCEQELFDLLHCLDHCHSQ |
| SEQ ID NO: 76 | GSREGCASRCTKYNAELEKCEARVVSMSNTEEDCEQELEDLLHCVDHCHSQ |
| SEQ ID NO: 77 | GSREGCASRCTKYNAELEKCEARVVSMSNTEEDCEQELEDLLHCLDHCVSQ |
| SEQ ID NO: 78 | GSREGCASRCTKYNAELEKCEARVMSMSNTEETCVQELEDLLHCLDHCHSQ |
| SEQ ID NO: 79 | GSREGCASRCTKYNAELEKCEARVMSMSNTEETCEQELFDLLHCLDHCHSQ |
| SEQ ID NO: 80 | GSREGCASRCTKYNAELEKCEARVMSMSNTEETCEQELEDLLHCVDHCHSQ |
| SEQ ID NO: 81 | GSREGCASRCTKYNAELEKCEARVMSMSNTEETCEQELEDLLHCLDHCVSQ |
| SEQ ID NO: 82 | GSREGCASRCTKYNAELEKCEARVMSMSNTEEDCVQELFDLLHCLDHCHSQ |
| SEQ ID NO: 83 | GSREGCASRCTKYNAELEKCEARVMSMSNTEEDCVQELEDLLHCVDHCHSQ |
| SEQ ID NO: 84 | GSREGCASRCTKYNAELEKCEARVMSMSNTEEDCVQELEDLLHCLDHCVSQ |
| SEQ ID NO: 85 | GSREGCASRCTKYNAELEKCEARVMSMSNTEEDCEQELFDLLHCVDHCHSQ |
| SEQ ID NO: 86 | GSREGCASRCTKYNAELEKCEARVMSMSNTEEDCEQELFDLLHCLDHCVSQ |
| SEQ ID NO: 87 | GSREGCASRCTKYNAELEKCEARVMSMSNTEEDCEQELEDLLHCVDHCVSQ |
| SEQ ID NO: 88 | GSREGCASRCMKYNAELEKCEARVMSMSNTEEDCEQELEDLLHCLDHCHSQ |
| SEQ ID NO: 89 | GSREGCASRCTKYNDELEKCEARVMSMSNTEEDCEQELEDLLHCLDHCHSQ |
| SEQ ID NO: 90 | GSREGCASRCTKYNAELEKCEARMMSMSNTEEDCEQELEDLLHCLDHCHSQ |
| SEQ ID NO: 91 | GSREGCASRCTKYNAELEKCEARVMSMSNTEEDCEQELEDLLYCLDHCHSQ |
| SEQ ID NO: 92 | GSREGCASRCMKYNDELEKCEARMMSMSNTEEDCEQELEDLLHCLDHCHSQ |
| SEQ ID NO: 93 | GSREGCASRCMKYNDELEKCEARVMSMSNTEEDCEQELEDLLYCLDHCHSQ |
| SEQ ID NO: 94 | GSREGCASRCMKYNAELEKCEARMMSMSNTEEDCEQELEDLLYCLDHCHSQ |
| SEQ ID NO: 95 | GSREGCASRCTKYNDELEKCEARMMSMSNTEEDCEQELEDLLYCLDHCHSQ |
| SEQ ID NO: 96 | GSREGCASRCMKYNDELEKCEARMMSMSNTEEDCEQELEDLLYCLDHCHSQ |
| SEQ ID NO: 97 | GSREGCASRCTRYNAELERCEARVSSMSNTEETCVQELFDLLHCVDHCVSQ |
| SEQ ID NO: 98 | GSREGCASRCTRYNAELERCEARVMSMSNTEEDCEQELEDLLHCLDHCHSQ |
| SEQ ID NO: 99 | GSREGCASRCTRYNAELERCEARVVSMSNTEEDCEQELEDLLHCLDHCHSQ |
| SEQ ID NO: 100 | GSREGCASRCTRYNAELERCEARVMSMSNTEETCEQELEDLLHCLDHCHSQ |
| SEQ ID NO: 101 | GSREGCASRCTRYNAELERCEARVMSMSNTEEDCVQELEDLLHCLDHCHSQ |
| SEQ ID NO: 102 | GSREGCASRCTRYNAELERCEARVMSMSNTEEDCEQELFDLLHCLDHCHSQ |
| SEQ ID NO: 103 | GSREGCASRCTRYNAELERCEARVMSMSNTEEDCEQELEDLLHCVDHCHSQ |
| SEQ ID NO: 104 | GSREGCASRCTRYNAELERCEARVMSMSNTEEDCEQELEDLLHCLDHCVSQ |
| SEQ ID NO: 105 | GSREGCASRCTRYNAELERCEARVVSMSNTEETCEQELEDLLHCLDHCHSQ |
| SEQ ID NO: 106 | GSREGCASRCTRYNAELERCEARVVSMSNTEEDCVQELEDLLHCLDHCHSQ |
| SEQ ID NO: 107 | GSREGCASRCTRYNAELERCEARVVSMSNTEEDCEQELFDLLHCLDHCHSQ |
| SEQ ID NO: 108 | GSREGCASRCTRYNAELERCEARVVSMSNTEEDCEQELEDLLHCVDHCHSQ |
| SEQ ID NO: 109 | GSREGCASRCTRYNAELERCEARVVSMSNTEEDCEQELEDLLHCLDHCVSQ |
| SEQ ID NO: 110 | GSREGCASRCTRYNAELERCEARVMSMSNTEETCVQELEDLLHCLDHCHSQ |
| SEQ ID NO: 111 | GSREGCASRCTRYNAELERCEARVMSMSNTEETCEQELFDLLHCLDHCHSQ |
| SEQ ID NO: 112 | GSREGCASRCTRYNAELERCEARVMSMSNTEETCEQELEDLLHCVDHCHSQ |
| SEQ ID NO: 113 | GSREGCASRCTRYNAELERCEARVMSMSNTEETCEQELEDLLHCLDHCVSQ |
| SEQ ID NO: 114 | GSREGCASRCTRYNAELERCEARVMSMSNTEEDCVQELFDLLHCLDHCHSQ |
| SEQ ID NO: 115 | GSREGCASRCTRYNAELERCEARVMSMSNTEEDCVQELEDLLHCVDHCHSQ |
| SEQ ID NO: 116 | GSREGCASRCTRYNAELERCEARVMSMSNTEEDCVQELEDLLHCLDHCVSQ |
| SEQ ID NO: 117 | GSREGCASRCTRYNAELERCEARVMSMSNTEEDCEQELFDLLHCVDHCHSQ |
| SEQ ID NO: 118 | GSREGCASRCTRYNAELERCEARVMSMSNTEEDCEQELFDLLHCLDHCVSQ |
| SEQ ID NO: 119 | GSREGCASRCTRYNAELERCEARVMSMSNTEEDCEQELEDLLHCVDHCVSQ |
| SEQ ID NO: 120 | GSREGCASRCMRYNAELERCEARVMSMSNTEEDCEQELEDLLHCLDHCHSQ |
| SEQ ID NO: 121 | GSREGCASRCTRYNDELERCEARVMSMSNTEEDCEQELEDLLHCLDHCHSQ |
| SEQ ID NO: 122 | GSREGCASRCTRYNAELERCEARMMSMSNTEEDCEQELEDLLHCLDHCHSQ |
| SEQ ID NO: 123 | GSREGCASRCTRYNAELERCEARVMSMSNTEEDCEQELEDLLYCLDHCHSQ |
| SEQ ID NO: 124 | GSREGCASRCMRYNDELERCEARMMSMSNTEEDCEQELEDLLHCLDHCHSQ |
| SEQ ID NO: 125 | GSREGCASRCMRYNDELERCEARVMSMSNTEEDCEQELEDLLYCLDHCHSQ |
| SEQ ID NO: 126 | GSREGCASRCMRYNAELERCEARMMSMSNTEEDCEQELEDLLYCLDHCHSQ |
| SEQ ID NO: 127 | GSREGCASRCTRYNDELERCEARMMSMSNTEEDCEQELEDLLYCLDHCHSQ |
| SEQ ID NO: 128 | GSREGCASRCMRYNDELERCEARMMSMSNTEEDCEQELEDLLYCLDHCHSQ |

In some embodiments, a TfR-binding peptide disclosed herein comprises
GSREGCAX₁RCX₂KYX₄DEX₂X₃KCX₃ARMMSMSNTEEDCEQEX₂EDX₂X₂YCX₂X₃X₅CX₅X₁X₄(SEQ ID NO: 306) or
REGCAX₁RCX₂KYX₄DEX₂X₃KCX₃ARMMSMSNTEEDCEQEX₂EDX₂X₂YCX₂X₃X₅CX₅X₁X₄ (SEQ ID NO: 325), wherein X₁ can be independently selected from S, T, D, or N, X₂ can be independently selected from A, M, I, L, or V, X₃ can be independently selected from D, E, N, Q, S, or T, X₄ can be independently selected from D, E, H, K, R, N, Q, S, or T, and X₅ can be independently selected from H, K, R, N, Q, S, or T.

In some embodiments, a TfR-binding peptide disclosed herein comprises
GSREX₁CX₂X₃RCX₄KYX₅DEX₆X₇KCX₈ARMMSMSNTEEDCEQELEDLLYCLDHCHSQ (SEQ ID NO: 307) or
REX₁CX₂X₃RCX₄KYX₅DEX₆X₇KCX₈ARMMSMSNTEEDCEQELEDLLYCLDHCHSQ (SEQ ID NO: 326), wherein X₁, X₂, X₃, X₄, X₅, X₆, X₇ and X₈ are TfR binding interface residues and can independently be any amino acid. In some embodiments, a TfR-binding peptide disclosed herein comprises
GSREGCASRCMKYNDELEKCEARMMSMSNTEEDCEQEXEDX₂X₃YCX₄XSX₆CX₇X₈X₉ (SEQ ID NO: 308) or
REGCASRCMKYNDELEKCEARMMSMSNTEEDCEQEXEDX₂X₃YCX₄XX₆CX₇X₈X₉ (SEQ ID NO: 327), wherein X₁, X₂, X₃, X₄, X₅, X₆, X₇, X₈, and X₉ are TfR binding interface residues and can independently be any amino acid. In some embodiments, a TfR-binding peptide disclosed herein comprises
GSREX₁CX₂X₃RCX₄KYX₅DEX₆X₇KCX₈ARMMSMSNTEEDCEQEX₉EDX₁₀X₁₁YCX₁₂X₁₃X₁₃CX₁₅X₁₆X₁₇ (SEQ ID NO: 309) or
REX₁CX₂X₃RCX₄KYX₅DEX₆X₇KCX₈ARMMSMSNTEEDCEQEX₉EDX₁₀X₁₁YCX₁₂X₁₃X₁₃C X₁₅X₁₆X₁₇ (SEQ ID NO: 328), wherein X₁, X₂, X₃, X₄, X₅, X₆, X₇, X₈, X₉, X₁₀, X₁₁, X₁₂, X₁₃, X₁₄, X₁₅, X₁₆ and X₁₇ are TfR binding interface residues and can independently be any amino acid. In some embodiments, a TfR-binding peptide disclosed herein comprises
GSREGCASRCMKYNDELEKCEARMMSMSNTEEDCEQELEDLLYCLDHCHSQ (SEQ ID NO: 96).

In some embodiments, a TfR-binding peptide disclosed herein comprises
X₁X₂X₃X₄GX₅ASX₆X₇MX₈X₉NX₁₀X₁₁LEX₁₂X₁₃EX₁₄X₁₅X₁₆X₁₇X₁₈X₁₉X₂₀X₂₁X₂₂X₂₃X₂₄X₂₅X₂₆X₂X₂₈X₂₉X₃₀X₃₁X₃₂X₃₃X₃₄X₃₅X₃₆X₃₇X₃₈X₃₉X₄₀X₄₁X₄₂X₄₃ (SEQ ID NO: 310), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, X₄₂, and X₄₃ can independently be any amino acid.

In some embodiments, a TfR-binding peptide disclosed herein comprises
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇LX₃₈X₃₉LLX₄₀X₄₁LDHX₄₂HSQ (SEQ ID NO: 311), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, and X₄₂ can independently be any amino acid.

In some embodiments, a TfR-binding peptide disclosed herein comprises
X₁X₂X₃X₄GX₅ASX₆X₇MX₈X₉NX₁₀X₁₁LEX₁₂X₁₃EX₁₄X₁₅X₁₆X₁₇X₁₈X₁₉X₂₀X₂₁X₂₂X₂₃X₂₄X₂₅X₂₆X₂₇X₂₈X₂₉LX₃₀X₃₁LLX₃₂X₃₃LDHX₃₄HSQ (SEQ ID NO: 312), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, and X₃₄ can independently be any amino acid.

In some embodiments, a TfR-binding peptide disclosed herein comprises at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% sequence homology to any one of SEQ ID NO: 1-SEQ ID NO: 134, or any variant, homolog, or functional fragment thereof. In some embodiments, a TfR-binding peptide disclosed herein comprises any one of SEQ ID NO: 1-SEQ ID NO: 134 or SEQ ID NO: 306-SEQ ID NO: 335, or any variant, homolog, or functional fragment thereof. In some embodiments, a peptide that binds to a TfR comprises the amino acid sequence set forth in SEQ ID NO: 96.

In some embodiments, a TfR-binding peptide comprises canonical amino acid residues as surface interface residues at any one of the corresponding positions 5, 7, 8, 14, 17, 18, 21, 38, 42, 45, 46, 47, 50, 51, with reference to SEQ ID NO: 96 or a combination thereof. In some embodiments, a TfR-binding peptide comprises canonical amino acid residues as surface interface residues at any one of the corresponding positions G5, A7, S8, N14, L17, E18, E21, L38, L42, L45, D46, H47, 550, Q51, with reference to SEQ ID NO: 96 or a combination thereof. In some embodiments, the peptide of the present disclosure comprises at least one or more of these corresponding residues in SEQ ID NO: 1-SEQ ID NO: 134. Such peptides can accordingly be engineered with enhanced binding to TfR. In some embodiments, a TfR-binding peptide disclosed herein comprises
X₁X₂X₃X₄GX₅ASX₆X₇X₈X₉X₁₀NX₁₁X₁₂LEX₁₃X₁₄EX₁₅X₁₆X₁₇X₁₈X₁₉X₂₀X₂₁X₂₂X₂₃X₂₄X₂₅X₂₆X₂₇X₂₈X₂₉X₃₀LX₃₁X₃₂X₃₃LX₃₄X₃₅LDHX₃₆X₃₇SQ (SEQ ID NO: 313), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, and X₃₇ can independently be any amino acid.

In some embodiments, surface-distal hydrophilic amino acid residues (e.g., D, E, H, K, R, N, Q, S, or T) present in the amino acid sequence of a peptide contribute to peptide solubility. In some embodiments, a peptide as disclosed herein comprises a hydrophilic amino acid residue at any one of the corresponding positions 3, 4, 9, 11, 15, 16, 19, 23, 26, 28, 29, 30, 31, 32, 33, 35, 36, 37, 39, 40, with reference to SEQ ID NO: 96, or any combination thereof. In some instances, a peptide of the present disclosure comprises hydrophilic amino acid residues at the following corresponding positions: R3, E4, R9, K12, D15, E16, K19, R23, S26, S28, N29, T30, E31, E32, D33, E35, Q36, E37, E39, D40, with reference to SEQ ID NO: 96, or any combination thereof. In some embodiments, any one of or any combination of corresponding positions R3, E4, R9, K12, D15, E16, K19, R23, S26, S28, N29, T30, E31, E32, D33, E35, Q36, E37, E39, D40 with reference to SEQ ID NO: 96, can be mutated to another hydrophilic residue without significantly impacting solubility or TfR-binding. In some embodiments, a TfR-binding peptide disclosed herein comprises
X₁X₂REX₃X₄X₅X₆RX₇X₈KX₉X₁₀DEX₁₁X₁₂KX₁₃X₁₄X₁₅RX₁₆X₁₇SX₁₈SNTEEDX₁₉EQEX₂₀EDX₂₁X₂₂X₂₃X₂₄X₂₅X₂₆X₂₇X₂₈X₂₉X₃₀X₃₁ (SEQ ID NO: 314), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, and X₃₁ can independently be any amino acid. In some embodiments, a TfR-binding peptide disclosed herein comprises
GSX₁X₂GCASX₃CMX₄YNX₅X₆LEX₇CEAX₈MMX₉MX₁₀X₁₁X₁₂X₁₃X₁₄X₁₅CX₁₆X₁₇X₁₈LX₁₉X₂₀LLYCLDHCHSQ (SEQ ID NO: 315) or
X₁X₂GCASX₃CMX₄YNX₅X₆LEX₇CEAX₈MMX₉MX₁₀X₁₁X₁₂X₁₃X₁₄X₁₅CX₁₆X₁₇X₁₈LX₁₉X₂₀L LYCLDHCHSQ (SEQ ID NO: 329), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, and X₂₀ can be independently selected from D, E, H, K, R, N, Q, S, or T.

In some embodiments, a peptide of the present disclosure comprises hydrophilic residues (e.g., D, E, H, K, R, N, Q, S, or T) at corresponding positions 15, 35, 39, 49, with reference to SEQ ID NO: 96, or any combination thereof. In some instances, a peptide of the present disclosure comprises hydrophilic amino acid residues at the following corresponding positions: D15, E35, E39, H49, with reference to SEQ ID NO: 96, or any combination thereof. In some embodiments, any one of or any combination of corresponding positions D15, E35, E39, H49 with reference to SEQ ID NO: 96, can be mutated to another hydrophilic residue without significantly impacting solubility or TfR-binding. In some embodiments, a TfR-binding peptide disclosed herein comprises. In some embodiments, a TfR-binding peptide disclosed herein comprises
X₁X₂X₃X₄X₅X₆X₇X₈X₉X₁₀X₁₁X₁₂X₁₃X₁₄DX₁₅X₁₆X₁₇X₁₈X₁₉X₂₀X₂₁X₂₂X₂₃X₂₄X₂₅X₂₆X₂₇X₂₈X₂₉X₃₀X₃₁X₃₂X₃₃EX₃₄X₃₅X₃₆EX₃₇X₃₈X₃₉X₄₀X₄₁X₄₂X₄₃X₄₄X₄₅HX₄₆X₄₇ (SEQ ID NO: 316), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, X₄₂, X₄₃, X₄₄, X₄₅, X₄₆, and X₄₇ can independently be any amino acid. In some embodiments, a TfR-binding peptide disclosed herein comprises
GSREGCASRCMKYNX₁ELEKCEARMMSMSNTEEDCX₂QELX₃DLLYCLDHCX₄SQ (SEQ ID NO: 317) or
REGCASRCMKYNX₁ELEKCEARMMSMSNTEEDCX₂QELX₃DLLYCLDHCX₄SQ (SEQ ID NO: 330), wherein X₁, X₂, X₃, and X₄ can be independently selected from D, E, H, K, R, N, Q, S, or T.

In some embodiments, a peptide of the present disclosure comprises hydrophobic residues (e.g., A, M, I, L, V, F, W, or Y) at corresponding positions 15, 35, 39, 49, with reference to SEQ ID NO: 96, or any combination thereof. In some embodiments, a TfR-binding peptide disclosed herein comprises
GSREGCASRCMKYNX₁ELEKCEARMMSMSNTEEDCX₂QELX₃DLLYCLDHCX₄SQ (SEQ ID NO: 318) or
REGCASRCMKYNX₁ELEKCEARMMSMSNTEEDCX₂QELX₃DLLYCLDHCX₄SQ (SEQ ID NO: 331), wherein X₁, X₂, X₃, and X₄ can be independently selected from A, M, I, L, V, F, W, or Y. In some embodiments, hydrophilic amino acid residues at any one of the corresponding positions 15, 35, 39, and 49, with reference to SEQ ID NO: 96, are associated with higher binding affinity for TfR (e.g., target engagement) and higher solubility. In some embodiments, mutation of an amino acid residue at any one of the corresponding positions 15, 35, 39, and 49, with reference to SEQ ID NO: 96, from a hydrophobic to a hydrophilic residue can lead to higher binding affinity for TfR (e.g., target engagement) and higher solubility.

In some embodiments, a peptide of the present disclosure comprises hydrophobic residues (e.g., A, M, I, L, V, F, W, or Y) at corresponding positions 11, 25, 27, with reference to SEQ ID NO: 96, or any combination thereof. In some embodiments, a peptide of the present disclosure comprises hydrophilic residues (e.g., D, E, H, K, R, N, Q, S, or T) at corresponding positions 11, 25, 27, with reference to SEQ ID NO: 96, or any combination thereof. In some embodiments, hydrophobic amino acid residues at any one of the corresponding positions 11, 25, and 27, with reference to SEQ ID NO: 96, are associated with higher binding affinity for TfR (e.g., target engagement) and higher solubility. In some embodiments, mutation of an amino acid residue at any one of the corresponding positions 11, 25, and 27, with reference to SEQ ID NO: 96, from a hydrophilic residue to a hydrophobic residue can lead to higher binding affinity for TfR (e.g., target engagement) and higher solubility. In some embodiments, a peptide of the present disclosure comprises hydrophobic amino acid residues at the corresponding positions M11, M25, M27, with reference to SEQ ID NO: 96, or any combination thereof. In some instances, a peptide comprises the hydrophobic amino acid residues at the corresponding positions M11, M25, and M27, with reference to SEQ ID NO: 96. In some embodiments, any combination of the corresponding positions M11, M25, and M27, with reference to SEQ ID NO: 96, can be mutated to another hydrophobic residue without significantly impacting solubility or TfR-binding. In some embodiments, a TfR-binding peptide disclosed herein comprises
X₁X₂X₃X₄X₅X₆X₇X₈X₉X₁₀MX₁₁X₁₂X₁₃X₁₄X₁₅X₁₆X₁₇X₁₈X₁₉X₂₀X₂₁X₂₂X₂₃MX₂₄MX₂₅X₂₆X₂X₂₈X₂₉X₃₀X₃₁X₃₂X₃₃X₃₄X₃₅X₃₆X₃₇X₃₈X₃₉X₄₀X₄₁X₄₂X₄₃X₄₄X₄₅X₄₆X₄₇X₄₈ (SEQ ID NO: 319),
wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, X₄₂, X₄₃, X₄₄, X₄₅, X₄₆, X₄₇, and X₄₈ can independently be any amino acid. In some embodiments, a TfR-binding peptide disclosed herein comprises
GSREGCASRCX₁KYNDELEKCEARMX₂SX₃SNTEEDCEQELEDLLYCLDHCHSQ (SEQ ID NO: 320) or
REGCASRCX₁KYNDELEKCEARMX₂SX₃SNTEEDCEQELEDLLYCLDHCHSQ (SEQ ID NO: 332), wherein X₁, X₂, and X₃ can be independently selected from A, M, I, L, V, F, W, or Y.

In some embodiments, a TfR-binding peptide disclosed herein comprises
GSREGCASRCX₁KYNDELEKCEARMX₂SX₃SNTEEDCEQELEDLLYCLDHCHSQ (SEQ ID NO: 321) or
REGCASRCX₁KYNDELEKCEARMX₂SX₃SNTEEDCEQELEDLLYCLDHCHSQ (SEQ ID NO: 333), wherein X₁, X₂, and X₃ can be independently selected from D, E, H, K, R, N, Q, S, or T.

In some embodiments, a peptide of the present disclosure comprises an aliphatic amino acid residue (e.g., A, M, I, L, or V) at corresponding position 45, with reference to SEQ ID NO: 96. In some embodiments, a peptide of the present disclosure comprises an aromatic amino acid residue (e.g., F, W, or Y) at corresponding position 45. In some embodiments, an aliphatic amino acid residue at corresponding position 45 is associated with higher binding affinity to TfR. In some instances, a peptide comprises the aliphatic amino acid residue corresponding to L45, with reference to SEQ ID NO: 96. In some embodiments, mutation of an amino acid residue at corresponding position 45 from an aromatic residue to an aliphatic reside can lead to higher binding affinity for TfR (e.g., target engagement) and higher solubility. In some embodiments, mutating corresponding position L45 to another aliphatic residue may not significantly impact solubility or TfR-binding. In some embodiments, a TfR-binding peptide disclosed herein comprises
X₁X₂X₃X₄X₅X₆X₇X₈X₉X₁₀X₁₁X₁₂X₁₃X₁₄X₁₅X₁₆X₁₇X₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇X₃₈X₃₉X₄₀X₄₁X₄₂X₄₃X₄₄LX₄₅X₄₆X₄₇X₄₈X₄₉X₅₀ (SEQ ID NO: 322),
wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, X₄₂, X₄₃, X₄₄, X₄₅, X₄₆, X₄₇, X₄₈, X₄₉, and X₅₀ can independently be any amino acid. In some embodiments, a TfR-binding peptide disclosed herein comprises
GSREGCASRCMKYNDELEKCEARMMSMSNTEEDCEQELEDLLYCX₁DHCHSQ (SEQ ID NO: 323) or REGCASRCMKYNDELEKCEARMMSMSNTEEDCEQELEDLLYCX₁DHCHSQ (SEQ ID NO: 334), wherein X₁ can be independently selected from A, M, I, L, or V.

In some embodiments, a peptide of the present disclosure comprises
GSREGCASRCMX₁YNDELEX₂CEARMMSMSNTEEDCEQELEDLLYCLDHCHSQ (SEQ ID NO: 324) or
REGCASRCMX₁YNDELEX₂CEARMMSMSNTEEDCEQELEDLLYCLDHCHSQ (SEQ ID NO: 335), wherein X₁ and X₂ can be independently selected from K or R. In some embodiments, these residues at corresponding position 12 and 19, with reference to SEQ ID NO: 96, can be used for chemical conjugation to another molecule (e.g., an active or a detectable agent). In some embodiments, X₁ and X₂ are both R and chemical conjugation occurs at the N-terminus of the peptide.

In some embodiments, mutations in any one or more of the amino acid residues of a peptide of the present disclosure can improve binding affinity of the peptide to TfR. In some embodiments, mutations in 5-80% of amino acid residues of a peptide of the present disclosure improve the binding affinity of the peptide to TfR. In some embodiments, mutations in 1-100%, 5-100%, or 5-50% of amino acid residues of a peptide of the present disclosure improve binding affinity of the peptide to TfR. In some embodiments, mutations in 15-50% of amino acid residues of a peptide of the present disclosure improve binding affinity of the peptide to TfR. In some embodiments, mutations in 15-30% of amino acid residues of a peptide of the present disclosure improve binding affinity of the peptide to TfR. In some embodiments, mutations in 25-30% of amino acid residues of a peptide of the present disclosure improve binding affinity of the peptide to TfR. For example, mutations in 14 of the 51 amino acid residues (27.5%) of a peptide having a sequence of SEQ ID NO: 96 can improve binding affinity of the peptide to TfR.

In some embodiments, mutations in any one or more of the amino acid residues of a peptide of the present disclosure can lie at the binding interface of TfR. In some embodiments, a mutation to a peptide can improve binding affinity, which can be beneficial to binding and transcytosis of a peptide disclosed herein. In some embodiments, the peptides provided herein can have many mutations or few mutations to obtain optimal activity, wherein optimal activity is sufficient binding for engagement of the TfR, but not necessarily binding that is so strong as to preclude release of the peptide after transcytosis. Thus, peptides of the present disclosure can comprise a number of mutations (also referred to as % mutated amino acid residues) that tune binding affinity and off rate to obtain optimal binding, function (e.g., transcytosis, BBB-penetration, cell membrane penetration, transport across a biological barrier), and release of the peptide. Thus, mutations that result in the highest possible affinity may not necessarily correlate to a superior peptide having optimal binding and transcytosis.

In some embodiments, 1-100% or 5-100% of amino acid residues of a peptide of the present disclosure lie at the binding interface of TfR. In some embodiments, 10-90% of amino acid residues of a peptide of the present disclosure lie at the binding interface of TfR. In some embodiments, 20-80% of amino acid residues of a peptide of the present disclosure lie at the binding interface of TfR. In some embodiments, 30-70% of amino acid residues of a peptide of the present disclosure lie at the binding interface of TfR. In some embodiments, 40-60% of amino acid residues of a peptide of the present disclosure lie at the binding interface of TfR. In some embodiments, 30-35% of amino acid residues of a peptide of the present disclosure lie at the binding interface of TfR. For example, 17 of the 51 amino acid residues (33%) of a peptide having a sequence of SEQ ID NO: 96 can lie at the binding interface of TfR.

In some embodiments, mutations in any one or more of the amino acid residues of a peptide of the present disclosure that lie at the binding interface of TfR can improve binding affinity of the peptide to TfR. In some embodiments, mutations in 1-100% or 5-100% of amino acid residues of a peptide of the present disclosure that lie at the binding interface of TfR improve binding affinity of the peptide to TfR. In some embodiments, mutations in 5-80% of amino acid residues of a peptide of the present disclosure that lie at the binding interface of TfR improve binding affinity of the peptide to TfR. In some embodiments, mutations in 10-70% of amino acid residues of a peptide of the present disclosure that lie at the binding interface of TfR improve binding affinity of the peptide to TfR. In some embodiments, mutations in 15-60% of amino acid residues of a peptide of the present disclosure that lie at the binding interface of TfR improve binding affinity of the peptide to TfR. In some embodiments, mutations in 20-50% of amino acid residues of a peptide of the present disclosure that lie at the binding interface of TfR improve binding affinity of the peptide to TfR. In some embodiments, mutations in 25-30% of amino acid residues of a peptide of the present disclosure that lie at the binding interface of TfR improve binding affinity of the peptide to TfR. For example, mutations in 5 of the 17 amino acid residues (29%) of a peptide having a sequence of SEQ ID NO: 96 that lie at the binding interface of TfR and can improve binding affinity of the peptide to TfR.

In some embodiments, mutations in any one or more of the amino acid residues of a peptide of the present disclosure are distal to the binding interface of TfR. In some embodiments, 1-100% or 5-100% of amino acid residues of a peptide of the present disclosure are distal to the binding interface of TfR. In some embodiments, 10-90% of amino acid residues of a peptide of the present disclosure are distal to the binding interface of TfR. In some embodiments, 20-80% of amino acid residues of a peptide of the present disclosure are distal to the binding interface of TfR. In some embodiments, 30-70% of amino acid residues of a peptide of the present disclosure are distal to the binding interface of TfR. In some embodiments, 40-60% of amino acid residues of a peptide of the present disclosure are distal to the binding interface of TfR. In some embodiments, 65-70% of amino acid residues of a peptide of the present disclosure are distal to the binding interface of TfR. For example, 34 of the 51 amino acid residues (66%) of a peptide having a sequence of SEQ ID NO: 96 can lie at the binding interface of TfR.

In some embodiments, mutations in any one or more of the amino acid residues of a peptide of the present disclosure are distal to the binding interface of TfR improve binding affinity of the peptide to TfR. In some embodiments, mutations in 1-100% or 5-100% of amino acid residues of a peptide of the present disclosure that are distal to the binding interface of TfR improve binding affinity of the peptide to TfR. In some embodiments, mutations in 5-80% of amino acid residues of a peptide of the present disclosure that are distal to the binding interface of TfR improve binding affinity of the peptide to TfR. In some embodiments, mutations in 10-70% of amino acid residues of a peptide of the present disclosure that are distal to the binding interface of TfR improve binding affinity of the peptide to TfR. In some embodiments, mutations in 15-60% of amino acid residues of a peptide of the present disclosure that are distal to the binding interface of TfR improve binding affinity of the peptide to TfR. In some embodiments, mutations in 20-50% of amino acid residues of a peptide of the present disclosure that are distal to the binding interface of TfR improve binding affinity of the peptide to TfR. In some embodiments, mutations in 25-30% of amino acid residues of a peptide of the present disclosure that are distal to the binding interface of TfR improve binding affinity of the peptide to TfR. For example, mutations in 5 of the 17 amino acid residues that are distal to the binding interface of TfR can improve binding affinity of the peptide to TfR. For example, mutations in 9 of the 34 amino acid residues (26.5%) of a peptide having a sequence of SEQ ID NO: 96 that are distal to the binding interface of TfR can improve binding affinity of the peptide to TfR. In some embodiments, and without being bound to any theory, one or more mutations in the amino acid residues of the peptide that are distal to the binding interface of TfR can improve protein folding, enhance protein solubility, and/or alter the backbone geometry that can improve binding through an optimized interface shape complementarity.

In some embodiments, a peptide of the present disclosure can comprise a sequence having cysteine residues at one or more of positions 11, 12, 13, 14, 19, 20, 21, 22, 36, 38, 39, 41. In some embodiments, a peptide comprises Cys at positions 11, 12, 19, 20, 36, 39, or any combination thereof. For example, in certain embodiments, a peptide can comprise a sequence having a cysteine residue at position 11. In certain embodiments, a peptide can comprise a sequence having a cysteine residue at position 12. In certain embodiments, a peptide can comprise a sequence having a cysteine residue at position 13. In certain embodiments, a peptide can comprise a sequence having a cysteine residue at position 14. In certain embodiments, a peptide can comprise a sequence having a cysteine residue at position 19. In certain embodiments, a peptide can comprise a sequence having a cysteine residue at position 20. In certain embodiments, a peptide can comprise a sequence having a cysteine residue at position 21. In certain embodiments, a peptide can comprise a sequence having a cysteine residue at position 22. In certain embodiments, a peptide can comprise a sequence having a cysteine residue at position 36. In certain embodiments, a peptide can comprise a sequence having a cysteine residue at position 38. In certain embodiments, a peptide can comprise a sequence having a cysteine residue at position 39. In certain embodiments, a peptide can comprise a sequence having a cysteine residue at position 41. In some embodiments, the first cysteine residue in the sequence can be disulfide bonded with the 4th cysteine residue in the sequence, the 2nd cysteine residue in the sequence can be disulfide bonded to the 5th cysteine residue in the sequence, and the 3rd cysteine residue in the sequence can be disulfide bonded to the 6th cysteine residue in the sequence. Optionally, a peptide can comprise one disulfide bridge that passes through a ring formed by two other disulfide bridges, also known as a “two-and-through” structure system. In some embodiments, the peptides disclosed herein can have one or more cysteines mutated to serine.

In some embodiments, peptides of the present disclosure comprise at least one cysteine residue. In some embodiments, peptides of the present disclosure comprise at least two cysteine residues. In some embodiments, peptides of the present disclosure comprise at least three cysteine residues. In some embodiments, peptides of the present disclosure comprise at least four cysteine residues. In some embodiments, peptides of the present disclosure comprise at least five cysteine residues. In some embodiments, peptides of the present disclosure comprise at least six cysteine residues. In some embodiments, peptides of the present disclosure comprise at least ten cysteine residues. In some embodiments, a peptide of the present disclosure comprises six cysteine residues.

In some embodiments, a peptide of the present disclosure comprises an amino acid sequence having cysteine residues at one or more positions. In some embodiments, the one or more cysteine residues are located at any one of the amino acid positions 6, 10, 20, 34, 44, 48, or any combination thereof. In some aspects of the present disclosure, the one or more cysteine (C) residues participate in disulfide bonds with various pairing patterns (e.g., C10-C20). In some embodiments, the pairing patterns are C6-C48, C10-C44, and C20-C34. In some embodiments, the peptides as described herein comprise at least one, at least two, or at least three disulfide bonds. In some embodiments, at least one, at least two, or at least three disulfide bonds are arranges according to the C6-C48, C10-C44, and C20-C34 pairing patterns, or a combination thereof. In some embodiments, peptides as described herein comprise three disulfide bonds with the pairing patterns C6-C48, C10-C44, and C20-C34.

In certain embodiments, a peptide comprises a sequence having a cysteine residue at position 6. In certain embodiments, a peptide comprises a sequence having a cysteine residue at position 10. In certain embodiments, a peptide comprises a sequence having a cysteine residue at position 20. In certain embodiments, a peptide comprises a sequence having a cysteine residue at position 34. In certain embodiments, a peptide comprises a sequence having a cysteine residue at position 44. In certain embodiments, a peptide comprises a sequence having a cysteine residue at position 50. In some embodiments, the first cysteine residue in the sequence is disulfide bonded with the last cysteine residue in the sequence. In some embodiments, the second cysteine residue in the sequence is disulfide bonded with the second to the last cysteine residue in the sequence. In some embodiments, the third cysteine residue in the sequence is disulfide bonded with the third to the last cysteine residue in the sequence and so forth.

In some embodiments, the first cysteine residue in the sequence is disulfide bonded with the 6th cysteine residue in the sequence, the 2nd cysteine residue in the sequence is disulfide bonded to the 5th cysteine residue in the sequence, and the 3rd cysteine residue in the sequence is disulfide bonded to the 4th cysteine residue in the sequence. Optionally, a peptide can comprise one disulfide bridge that passes through a ring formed by two other disulfide bridges, also known as a “two-and-through” structure system. In some embodiments, the peptides disclosed herein have one or more cysteines mutated to serine.

In some embodiments, a peptide comprises no cysteine or disulfides. In some embodiments, a peptide comprises 1 or more, 2 or more, 3 or more, 4 or more, 5 or more, 6 or more, 7 or more, 8 or more, 9 or more, 10 or more, 11 or more, 12 or more, 13 or more, 14 or more, or 15 or more cysteine or disulfides. In other embodiments, 1 or more, 2 or more, 3 or more, 4 or more, 5 or more, 6 or more, 7 or more, 8 or more, 9 or more, 10 or more cysteine residues have been replaced with serine residues. In some embodiments, 1 or more, 2 or more, 3 or more, 4 or more, 5 or more, 6 or more, 7 or more, 8 or more, 9 or more, 10 or more cysteine residues have been replaced with threonine residues.

In some embodiments, a peptide comprises no Cys or disulfides. In some embodiments, a peptide comprises 1 or more, 2 or more, 3 or more, 4 or more, 5 or more, 6 or more, 7 or more, 8 or more, 9 or more, 10 or more, 11 or more, 12 or more, 13 or more, 14 or more, or 15 or more Cys or disulfides. In other embodiments, 1 or more, 2 or more, 3 or more, 4 or more, 5 or more, 6 or more, 7 or more, 8 or more, 9 or more, 10 or more Cys residues have been replaced with Ser residues. In some embodiments, 1 or more, 2 or more, 3 or more, 4 or more, 5 or more, 6 or more, 7 or more, 8 or more, 9 or more, 10 or more Cys residues have been replaced with Thr residues.

In some instances, one or more or all of the methionine residues in the peptide are replaced by leucine or isoleucine. In some instances, one or more or all of the tryptophan residues in the peptide are replaced by phenylalanine or tyrosine. In some instances, one or more or all of the asparagine residues in the peptide are replaced by glutamine. In some embodiments, the N-terminus of the peptide is blocked, such as by an acetyl group. Alternatively or in combination, in some instances, the C-terminus of the peptide is blocked, such as by an amide group. In some embodiments, the peptide is modified by methylation on free amines.

For example, full methylation can be accomplished through the use of reductive methylation with formaldehyde and sodium cyanoborohydride.

The peptide oligonucleotide complexes of the present disclosure include TfR-binding peptides and peptide variants within the peptide oligonucleotide complex. In some embodiments, a TfR-binding peptide is a CDP comprising six cysteine residues. In some instances, the six cysteines of a TfR-binding CDP correspond to residues C6, C10, C20, C34, C44, and C48, with reference to SEQ ID NO: 96 (C4, C8, C18, C32, C42, and C46, with reference to 32), participate in disulfides, and thus contribute to peptide stability.

The surface interface residues of a TfR-binding CDP may correspond to residues G5, A7, S8, N14, L17, E18, E21, L38, L42, L45, D46, H47, S50, Q51, with reference to SEQ ID NO: 96 (G3, A5, S6, N12, L15, E16, E19, L36, L40, L43, D44, H45, S48, Q49, with reference to SEQ ID NO: 32), and may contribute to TfR-binding. In some embodiments, the peptide of the present disclosure comprises at least one or more of these corresponding residues in SEQ ID NO: 1-SEQ ID NO: 134. Such peptides can accordingly be engineered with enhanced binding to TfR.

Hydrophilic surface-distal residues such as D, E, H, K, R, N, Q, S, or T may contribute to peptide solubility corresponding to the following amino acid residues R3, E4, R9, K12, D14, E15, K19, R23, S26, S28, N29, T30, E31, E32, D33, E35, Q36, E37, E39, and D40, with reference to SEQ ID NO: 96 (R1, E3, R7, K10, D12, E13, K17, R21, S24, S26, N27, T28, E29, E30, D31, E33, Q34, E35, E37, and D38, with reference to SEQ ID NO: 32). In some embodiments, the peptide of the present disclosure comprises at least one or more of these corresponding residues in SEQ ID NO: 1-SEQ ID NO: 134. Such peptides can accordingly be engineered with enhanced solubility.

Higher binding affinity may be associated with the presence of hydrophilic residues such as D, E, H, K, R, N, Q, S, or T as shown by improved binding from a mutation away from a nonpolar or hydrophobic residue such as A, M, I, L, V, F, W, or Y at the residues corresponding to D15, E35, E39, and H49, with reference to SEQ ID NO: 96 (D13, E33, E37, and H47, with reference to SEQ ID NO: 32). In some embodiments, the peptide of the present disclosure comprises at least one or more of these corresponding residues in SEQ ID NO: 1-SEQ ID NO: 134. Such peptides can accordingly be engineered with modified binding affinity.

Higher binding affinity to TfR may be associated with nonpolar or hydrophobic residues such as A, M, I, L, V, F, W, or Y as shown by improved binding from a mutation away from a hydrophilic residue such as D, E, H, K, R, N, Q, S, or T at the amino acid residues corresponding to M11, M25, and M27, with reference to SEQ ID NO: 96 (M9, M23, and M25, with reference to SEQ ID NO: 32). In some embodiments, the peptide of the present disclosure comprises at least one or more of these corresponding residues in SEQ ID NO: 1-SEQ ID NO: 134. Such peptides can accordingly be engineered with modified binding affinity.

A higher TfR-binding affinity may be associated with aliphatic residues such as A, M, I, L, or V as shown by improved binding from a mutation away from a large, aromatic residues such as F, W, or Y at the amino acid residue corresponding to L45 with reference to SEQ ID NO: 96 (L43 with reference to SEQ ID NO: 32). Substitutions of any one or more F, W, or Y in a peptide of the present disclosure to an aliphatic residue comprising A, M, I, L, or V can be sued to enhance the binding affinity of the peptide to TfR.

Any of peptides of the present disclosure (e.g., any one of SEQ ID NO: 1-SEQ ID NO: 134 or SEQ ID NO: 306-SEQ ID NO: 335) can be modified at one or more of the corresponding residues described herein, to generate peptide variants with improved properties including enhanced stability and increased (or decreased) binding properties or modified TfR-binding affinity and increased (or decreased) transcytosis properties, including modified kₐ(association) and k_{d} (dissociation) rate constants.

Sequence alignments of certain TfR-binding peptides are shown in TABLE 2. Certain residues involved in the interaction with TfR are shown in bold. Surface interacting residues include but are not limited to those indicated. In some embodiments the TfR-binding peptides within the peptide oligonucleotide complexes are conserved in one or more of the residues, and up to all such residues, involved in the interaction with TfR as are shown in bold in TABLE 2.

TABLE 2
Corresponding Residues in TfR-Binding Peptides within Peptide
oligonucleotide Complexes

| SEQ ID NO | Sequence |
|---|---|
| SEQ ID NO: 1 | REGCASRCTKYNAELEKCEARVSSMSNTEETCVQELFDLLHCVDHCVSQ |
| SEQ ID NO: 2 | REGCASRCTKYNAELEKCEARVMSMSNTEEDCEQELEDLLHCLDHCHSQ |
| SEQ ID NO: 30 | REGCASRCMKYNAELEKCEARMMSMSNTEEDCEQELEDLLYCLDHCHSQ |
| SEQ ID NO: 32 | REGCASRCMKYNDELEKCEARMMSMSNTEEDCEQELEDLLYCLDHCHSQ |
| SEQ ID NO: 33 | REGCASRCTRYNAELERCEARVSSMSNTEETCVQELFDLLHCVDHCVSQ |
| SEQ ID NO: 34 | REGCASRCTRYNAELERCEARVMSMSNTEEDCEQELEDLLHCLDHCHSQ |
| SEQ ID NO: 62 | REGCASRCMRYNAELERCEARMMSMSNTEEDCEQELEDLLYCLDHCHSQ |
| SEQ ID NO: 64 | REGCASRCMRYNDELERCEARMMSMSNTEEDCEQELEDLLYCLDHCHSQ |
| SEQ ID NO: 65 | GSREGCASRCTKYNAELEKCEARVSSMSNTEETCVQELFDLLHCVDHCVSQ |
| SEQ ID NO: 66 | GSREGCASRCTKYNAELEKCEARVMSMSNTEEDCEQELEDLLHCLDHCHSQ |
| SEQ ID NO: 94 | GSREGCASRCMKYNAELEKCEARMMSMSNTEEDCEQELEDLLYCLDHCHSQ |
| SEQ ID NO: 96 | GSREGCASRCMKYNDELEKCEARMMSMSNTEEDCEQELEDLLYCLDHCHSQ |
| SEQ ID NO: 97 | GSREGCASRCTRYNAELERCEARVSSMSNTEETCVQELFDLLHCVDHCVSQ |
| SEQ ID NO: 98 | GSREGCASRCTRYNAELERCEARVMSMSNTEEDCEQELEDLLHCLDHCHSQ |
| SEQ ID NO: 126 | GSREGCASRCMRYNAELERCEARMMSMSNTEEDCEQELEDLLYCLDHCHSQ |
| SEQ ID NO: 128 | GSREGCASRCMRYNDELERCEARMMSMSNTEEDCEQELEDLLYCLDHCHSQ |

The peptide oligonucleotide complexes of the present disclosure include TfR binding peptide and peptide variants within the peptide oligonucleotide complex wherein the TfR-binding peptide portion is targeted to the CNS or to other TfR-expressing cells or tissues, and further contains an active agent or cargo moiety (e.g., a target-binding agent capable of binding a target molecule). In some embodiments, the peptide as described herein target and/or penetrate a Tf-expressing cellular layer or barrier and/or the membrane of a Tf-expressing cell. In some embodiments, a peptide targets and/or penetrates a cell membrane of a cell, wherein said cell is located in the CNS such as the brain. For example, a peptide construct comprising a TfR-binding peptide and one or more active agents (e.g., an oligonucleotide or other therapeutic or diagnostic compound) crosses a cellular barrier (e.g., BBB) via vesicular transcytosis, and subsequently targets and/or penetrates the cell membrane of a cell located within the CNS to deliver said one or more active agents to that cell.

In various embodiments, a peptide construct comprising a TfR-binding peptide as described herein and one or more active agents (e.g., a therapeutic or diagnostic compound) targets and/or penetrates the cell membrane of a TfR-expressing cell located in the gastrointestinal tract, spleen, liver, kidney, muscle, bone marrow, brain, or skin. In some cases, the TfR-expressing cell is a tumor cell, an immune cell, an erythrocyte, an erythrocyte precursor cell, a stem cell, a bone marrow cell, or stem cell. In some cases, the TfR-binding peptide is responsible for targeting the cell, e.g., in cases where the cell is overexpressing a TfR. In various embodiments, a peptide construct as described herein comprising a TfR-binding peptide conjugated to, linked to, or fused to one or more cargo molecules (e.g., an active and/or detectable agent) targets and/or penetrates the cell membrane of a cell located within various organs such as the spleen, brain, liver, kidney, muscle, bone marrow, gastrointestinal tract, or skin.

In some cases, the cargo molecule promotes the targeting of a specific cell, cell population, or tissue. In some cases, it is a combination of TfR-mediated cell targeting and cargo molecule promoted cell targeting. In some aspects, a peptide (e.g., peptide conjugate, fusion peptide, or a peptide within a peptide oligonucleotide complex) of the present disclosure is used to target said cell, cell population, or tissue in order to exert a certain biological (e.g., therapeutic) effect. In some aspects, a peptide of the present disclosure is used to deliver the cargo molecule into said cell to exert a certain biological effect.

The peptide oligonucleotide complexes of the present disclosure include TfR-binding peptide and peptide variants within the peptide oligonucleotide complex wherein the TfR-binding peptide portion further contains a tag that further enhances cell penetration or enhances delivery of the nucleotide to the target tissue or subcellular compartment. In some embodiments, peptides can comprise at least one or more tag peptide sequences for improved cell penetration. For example, peptides can comprise at least one or multiple Arg residues or residues from Tat protein for improved cell penetration property. Additional tag peptide sequences can include CysTat (CYRKKRRQRRR; SEQ ID NO: 141), S19-TAT (PFVIGAGVLGALGTGIGGIGRKKRRQRRR; SEQ ID NO: 142), R8 (RRRRRRRR; SEQ ID NO: 143), pAntp (RQIKIWFQNRRMKWKK; SEQ ID NO: 144), Pas-TAT (FFLIPKGGRKKRRQRRR; SEQ ID NO: 145), Pas-R8 (FFLIPKGRRRRRRRR; SEQ ID NO: 146), PasFHV (FFLIPKGRRRRNRTRRNRRRVR; SEQ ID NO: 147), Pas-pAntP (FFLIPKGRQIKIWFQNRRMKWKK; SEQ ID NO: 148), F2R4 (FFRRRR; SEQ ID NO: 149), B55 (KAVLGATKIDLPVDINDPYDLGLLLRHLRHHSNLLANIGDPAVREQVLSAMQEEE; SEQ ID NO: 150), auzurin (LSTAADMQGVVTDGMASGLDKDYLKPDD; SEQ ID NO: 151), IMT-P8 (RRWRRWNRFNRRRCR; SEQ ID NO: 152), BR2 (RAGLQFPVGRLLRRLLR; SEQ ID NO: 153), OMOTAG1 (KRAHHNALERKRR; SEQ ID NO: 154), OMOTAG2 (RRMKANARERNRM; SEQ ID NO: 155), pVEC (LLIILRRRIRKQAHAHSK; SEQ ID NO: 156), SynB3 (RRLSYSRRRF; SEQ ID NO: 157), DPV1047 (VKRGLKLRHVRPRVTRMDV; SEQ ID NO: 158), CY105Y (CSIPPEVKFNKPFVYLI; SEQ ID NO: 159), Transportan (GWTLNSAGYLLGKINLKALAALAKKIL; SEQ ID NO: 160), MTS (KGEGAAVLLPVLLAAPG; SEQ ID NO: 161), hLF (KCFQWQRNMRKVRGPPVSCIKR; SEQ ID NO: 162), PFVYLI (PFVYLI; SEQ ID NO: 163), yBBR (VLDSLEFIASKL, SEQ ID NO: 164), DRI-TAT31 (rrrqrrkkrgy, wherein the lowercase notation indicates D-amino acids; SEQ ID NO: 165), cyclic heptapeptide cyclo (cFΦR₄) (FΦRRRRQ, where Φ is 1-2-naphthylalanine, the entire moiety is cyclized, and Gln serves as a conjugation handle and can be substituted for other functional groups such as Lys (for amine coupling) or Cys (for sulfhydryl coupling); SEQ ID NO: 166), DPV3 (RKKRRRESRKKRRRES; SEQ ID NO: 171), C10H (RKGFYKRKQCKPSRGRKR; SEQ ID NO: 172), VP22 (NAATATRGRSAASRPTQRPRAPARSASRPRRPVQ; SEQ ID NO: 173), TP10 (AGYLLGKINLKALAALAKKIL; SEQ ID NO: 174), MAP (KLALKLALKALKAALKLA; SEQ ID NO: 175), BPrPp (MVKSKIGSWILVLFVAMWSDVGLCKKRP; SEQ ID NO: 176), ARF (MVRRFLVTLRIRRACGPPRVRV; SEQ ID NO: 177), GALA (WEAALAEALAEALAEHLAEALAEALEALAA; SEQ ID NO: 178), SAP (VRLPPPVRLPPPVRLPPP; SEQ ID NO: 179), MPG (GLAFLGFLGAAGSTMGAWSQPKKKRKV; SEQ ID NO: 180), Pep-1 (KETWWETWWTEWSQPKKKRKV; SEQ ID NO: 181), [WR]4 (WRWRWRWR; SEQ ID NO: 182), Ig(v) (MGLGLHLLVLAAALQGAKKKRKV; SEQ ID NO: 183), K-FGF (AAVALLPAVLLAHLLAP; SEQ ID NO: 184), Melittin (GIGAVLKVLTTGLPALISWIKRKRQQ; SEQ ID NO: 185), gH625 (HGLASTLTRWAHYNALIRAF; SEQ ID NO: 186), HIV-1 TAT protein (48-60) (GRKKRRQRRRPPQ; SEQ ID NO: 187), MPG HIV-gp41/SV40 T-antigen (GALFLGFLGAAGSTMGAWSQPKKKRKV; SEQ ID NO: 188), R6W3 (RRWWRRWRR; SEQ ID NO: 189), NLS (CGYGPKKKRKVGG; SEQ ID NO: 190), 8-lysines (KKKKKKKK; SEQ ID NO: 191), HRSV (RRIPNRRPRR; SEQ ID NO: 192), AIP6 (RLRWR; SEQ ID NO: 193), Pep-1 (KETWWETWWTEWSQPKKRKV; SEQ ID NO: 194), MAP17 (QLALQLALQALQAALQLA; SEQ ID NO: 195), VT5 (DPKGDPKGVTVTVTVTVTGKGDPKPD; SEQ ID NO: 196), Bac7 (RRIRPRPPRLPRPRPRPLPFPRPG; SEQ ID NO: 197), (PPR)n ((PPRPPRPPR; SEQ ID NO: 198), (PPRPPRPPRPPR; SEQ ID NO: 199), (PPRPPRPPRPPRPPR; SEQ ID NO: 200), (PPRPPRPPRPPRPPRPPR; SEQ ID NO: 201)), INF7 (GLFEAIEGFIENGWEGMIDGWYGC; SEQ ID NO: 202), CADY (GLWRALWRLLRSLWRLLWRA; SEQ ID NO: 203), Pep-7 (SDLWEMMMVSLACQY; SEQ ID NO: 204), TGN (TGNYKALHPHNG; SEQ ID NO: 205), Ku-70 (VPMLK; SEQ ID NO: 206), CPP (RRRRRGGRRRRRG; SEQ ID NO: 220) (RRRRRRGGRRRRRG; SEQ ID NO: 207), SVS-1 (KVKVKVKVDPPTKVKVKVK; SEQ ID NO: 208), L-CPP (LAGRRRRRRRRRK; SEQ ID NO: 209), RLW (RLWMRWYSPRTRAYG; SEQ ID NO: 210), K16ApoE (KKKKKKKKKKKKKKKKLRVRLASHLRKLRKRLLRDA; SEQ ID NO: 211), Angiopep-2 (TFFYGGSRGKRNNFKTEEY; SEQ ID NO: 212), ACPP (EEEEEEEEPLGLAGRRRRRRRRN; SEQ ID NO: 213), KAFAK (KAFAKLAARLYRKALARQLGVAA; SEQ ID NO: 214), hCT (9-32) (LGTYTQDFNKFHTFPQTAIGVGAP; SEQ ID NO: 215), VP22(version2) (DAATATRGRSAASRPTQRPRAPARSASRPRRPVE; SEQ ID NO: 216), MPG (GALFLGFLGAAGSTMGAWSQPKSKRKV; SEQ ID NO: 217), hPP3 (KPKRKRRKKKGHGWSR; SEQ ID NO: 218), PepNeg (SGTQEEY; SEQ ID NO: 219), CM18-TAT (KWKLFKKIGAVLKVLTTG; SEQ ID NO: 221), PTD4 (YARAAARQARA; SEQ ID NO: 222), or WaTx
(MKYFTLALTLLFLLLINPCKDMNFAWAESSEKVERASPQQAKYCYEQCNVNKVPFDQ CYQMCSPLERS; SEQ ID NO: 223). For example, in some embodiments, the peptide can comprise an Arginine patch (Arg patch), for example, an RRRRRRRR (SEQ ID NO: 143), or a variant or fragment thereof, sequence can be appended to either the N-terminus or the C-terminus of a peptide. In some embodiments, the Arg patch comprises two or more Arg residues, or Argₙ wherein n is a whole number and can be 2, 3, 4, 5, 6, 7, 8, 9, or 10 (SEQ ID NO: 167). In other embodiments, the peptide can comprise a Tat peptide (Tat proteins are reviewed in Gump et al. TAT transduction: the molecular mechanism and therapeutic prospects. Trends Mol Med. 2007 October; 13(10):443-8 and Harada et al. Antitumor protein therapy; application of the protein transduction domain to the development of a protein drug for cancer treatment. Breast Cancer. 2006; 13(1):16-26). The Tat peptide can have a sequence of, for example, YGRKKRRQRRR (SEQ ID NO: 168), GRKKRRQRRR (SEQ ID NO: 169), or any modification, variant, or fragment thereof, can be appended to the N-terminus or C-terminus of any TfR-binding peptide of the present disclosure. In some embodiments, the Tat peptide sequence can be GRKKRRQRRRPQ (SEQ ID NO: 170), GRKKRRQRRR (SEQ ID NO: 169), or a fragment or variant thereof. In some embodiments, the Tat peptide can be appended to the N-terminus of any TfR-binding peptide of the present disclosure following an N-terminal GS dipeptide and preceding, for example, a GGGS (SEQ ID NO: 234) spacer. In some embodiments, a cell-penetrating tag peptides, such as any one of SEQ ID NO: 141-SEQ ID NO: 233, can be appended to either the N-terminus or C-terminus of any peptide disclosed herein using a peptide linker such as GₓS_{y} (SEQ ID NO: 235) peptide linker, wherein x and y can be any whole number, such as 1, 2, 3, 4, 5, 6, 7, 8, 9, and 10. In other embodiments, a cell-penetrating peptide, such as a Tat peptide or an Arg patch, or any other moiety, can be appended to either the N-terminus or C-terminus of any peptide disclosed herein using a peptide linker such as GₓS_{y} (SEQ ID NO: 235) peptide linker, wherein x and y can be any whole number, such as 1, 2, 3, 4, 5, 6, 7, 8, 9, and 10. In some embodiments, the peptide linker comprises (GS)x (SEQ ID NO: 236), wherein x can be any whole number, such as 1, 2, 3, 4, 5, 6, 7, 8, 9, and 10. In some embodiments, the peptide linker comprises GGSSG (SEQ ID NO: 237), GGGGG (SEQ ID NO: 238), GSGSGSGS (SEQ ID NO: 239), GSGG (SEQ ID NO: 240), GGGGS (SEQ ID NO: 241), GGGS (SEQ ID NO: 234), GGS (SEQ ID NO: 242), GGGSGGGSGGGS (SEQ ID NO: 243), or a variant or fragment thereof. Additionally, KKYKPYVPVTTN (SEQ ID NO: 244) from DkTx, and EPKSSDKTHT (SEQ ID NO: 245) from human IgG3 can be used as a peptide linker. In other embodiments, the tag peptide can be appended to the peptide at any amino acid residue. In further embodiments, the tag peptide can be appended to the peptide at any amino acid residue without interfering with TfR-binding activity. In some embodiments, the tag peptide is appended via conjugation, linking, or fusion techniques. In other embodiments, the Tat peptide can be appended to the peptide at any amino acid residue. In further embodiments, the Tat peptide can be appended to the peptide at any amino acid residue without interfering with TfR-binding activity. In some embodiments, the Tat peptide is appended via conjugation, linking, or fusion techniques to a TfR-binding peptide to obtain a TfR-binding Cell Penetrating Peptide fusion (CPP fusion).

Cell-penetrating peptides include, but are not limited to, short amphipathic or cationic short peptides with a positive net charge and are capable of penetrating cellular membrane and transferring a molecular or cargo either covalently or non-covalently attached to the peptides into a cell. Such cell-penetrating peptides can be synthesized or derived from known proteins, such as penetratin, Tat peptide, pVEC, or chimeric peptides, such as transportan, MPG, Pep-1, or synthetic peptides, such as polyarginines, MAP, and R₆W₃.

In some embodiments, peptides can comprise at least one or more cell penetrating peptide sequences for improved cell penetration. For example, a cell penetrating peptide can include maurocaline (GDCLPHLKLCKENKDCCSKKCKRRGTNIEKRCR; SEQ ID NO: 224), imperatoxin (GDCLPHLKRCKADNDCCGKKCKRRGTNAEKRCR; SEQ ID NO: 225), hadrucalcin (SEKDCIKHLQRCRENKDCCSKKCSRRGTNPEKRCR; SEQ ID NO: 226), hemicalcin (GDCLPHLKLCKADKDCCSKKCKRRGTNPEKRCR; SEQ ID NO: 227), opicalcin-1 (GDCLPHLKRCKENNDCCSKKCKRRGTNPEKRCR; SEQ ID NO: 228), opicalcin-2 (GDCLPHLKRCKENNDCCSKKCKRRGANPEKRCR; SEQ ID NO: 229, midkine (62-104) (CKYKFENWGACDGGTGTKVRQGTLKKARYNAQCQETIRVTKPC; SEQ ID NO: 230), MCoTI-II (SGSDGGVCPKILKKCRRDSDCPGACICRGNGYCG; SEQ ID NO: 231), or chlorotoxin (MCMPCFTTDHQMARKCDDCCGGKGRGKCYGPQCLCR; SEQ ID NO: 232). In some embodiments, the cell penetrating peptide can have at least 80%, 90%, 95%, or 99% sequence identity with any sequence of SEQ ID NO: 141-SEQ ID NO: 233.

In some embodiments, a peptide is conjugated to, linked to, or fused to one or more cell-penetrating peptides, such as arginine-rich, amphipathic and lysine-rich, and hydrophobic residues or peptides capable of penetrating plasma membrane or nucleus for in vivo delivery of a protein or macromolecular cargo. Conjugation or fusion can be direct or with a spacer in between (chemical or peptide-based). A spacer can be any peptide linker. For example, a spacer can be GGGSGGSGGGS (SEQ ID NO: 246), KKYKPYVPVTTN (SEQ ID NO: 244) from DkTx, EPKSSDKTHT (SEQ ID NO: 245) from human IgG3 or any variant or fragment thereof. In some embodiments, the cell penetrating peptide sequence can be appended to either the N-terminus or the C-terminus of a peptide. In some embodiments, the cell penetrating peptide can be appended to the N-terminus of any TfR-binding peptide of the present disclosure following an N-terminal GS dipeptide and preceding, for example, a GGGS (SEQ ID NO: 234) spacer. In some embodiments, a cell-penetrating tag peptide, such as any one of SEQ ID NO: 141-SEQ ID NO: 233, can be appended to either the N-terminus or C-terminus of any peptide disclosed herein using a peptide linker such as GₓS_{y} (SEQ ID NO: 235) peptide linker, wherein x and y can be any whole number, such as 1, 2, 3, 4, 5, 6, 7, 8, 9, and 10. In some embodiments, the peptide linker comprises GGSSG (SEQ ID NO: 237), GGGGG (SEQ ID NO: 238), GSGSGSGS (SEQ ID NO: 239), GSGG (SEQ ID NO: 240), GGGGS (SEQ ID NO: 241), GGGS (SEQ ID NO: 234), GGS (SEQ ID NO: 242), GGGSGGGSGGGS (SEQ ID NO: 243), GGGSGGSGGGS (SEQ ID NO: 246), or a variant or fragment thereof. Additionally, KKYKPYVPVTTN (SEQ ID NO: 244) from DkTx, and EPKSSDKTHT (SEQ ID NO: 245) from human IgG3 can be used as a peptide linker. In other embodiments, the cell penetrating peptide can be appended to the peptide at any amino acid residue. In further embodiments, the cell penetrating peptide can be appended to the peptide at any amino acid residue without interfering with TfR-binding activity. In some embodiments, the cell penetrating peptide is appended via conjugation, linking, or fusion techniques. In other embodiments, the cell penetrating peptide can be appended to the peptide at any amino acid residue.

In other embodiments, cell penetration can be increased by using high dosage of a peptide described here, such as up to 10 μM, or 10 μM or more of the peptide. In some cases, an Arg patch can be fused, conjugated to, linked to, or co-delivered with a peptide. Up 10 μM, or 10 μM or more Arg patch can be co-delivered with a peptide to facilitate cell penetration. Protein transfection agents can also be used to increase cell penetration of a peptide. In some embodiments, direct cytosolic expression of a peptide can be used. In other embodiments, physical disruption methods such as electroporation can be used to improve delivery of a peptide into a cell.

In some embodiments, cell penetrance of a peptide described herein can be improved. For example, the binding interface of a peptide described herein can be grafted on a scaffold that is known to be cell-penetrant, such as a calcine. Similarly, sequences that are known to be cell penetrant can be grafted onto peptides of this disclosure. Some non-limiting examples of calcines can be imperatoxin-A, maurocalcine, hemicalcin, opiclacin 1, opicalcin 2, and hadrucalcin. The scaffold can comprise at least 60%, 70%, 80%, 90%, 95%, or 98% with any one of SEQ ID NO: 224-SEQ ID NO: 232. In some embodiments, the cell penetrance peptide can be calcines, modified calcines, derivatives of calcines, or fragments thereof, which can be used to increase cell penetration. Modified calcines, derivatives of calcines, or fragments can be screened for cell penetration activity such as activation of sarcoplasmic reticulum ryanodine receptors, activity on ryanodine-sensitive Ca²⁺ channels RyR1, Ryr2, or both, or as a selective agonist of the foregoing. Moreover, modified calcines can include substitution, addition or reduction of Lysine residues, or other charged residues, within a calcine in order to modify activity and optimize such calcine cell-penetration activity or activity on the RyR1 or RyR2 receptors. As an example, the six amino acid portion of helix 3 (MLICLF; SEQ ID NO: 233) can be transplanted onto a calcine or modified calcine scaffold to produce a bi-functional peptide that retains the cell penetration of the calcine with the novel TfR-binding function of the TfR-binding peptides. As another example, a peptide as described herein can have improved cell penetrating capabilities using cis-acting elements, including inclusion of K/R-rich sequences like TAT or octa-arginine, intra-helical arginine patches, or fusion to larger fragments of proteins identified in cell penetration screening like penetratin or melittin.

The peptide oligonucleotide complexes of the present disclosure include TfR-binding peptide and peptide variants within the peptide oligonucleotide complex wherein the TfR-binding peptide portion further contains a nuclear localization signal that further enhances cell penetration or enhances delivery of the nucleotide to the target tissue or subcellular compartment (e.g., the nucleus). In some embodiments, nuclear localization signals can be couple to, conjugated to, linked to, or fused to a peptide described herein to promote nuclear localization. In some embodiments, TfR-binding peptides are conjugated to, linked to, or fused to a nuclear localization signal, such as a four-residue sequence of K-K/R-X-K/R (SEQ ID NO: 299), wherein X can be any amino acid, or a variant thereof. In some embodiments, TfR-binding peptides are conjugated to, linked to, or fused to a nuclear localization signal as described in Lange et al, J Biol Chem. 2007 Feb. 23; 282(8):5101-5, such as PKKKRRV (SEQ ID NO: 300) or KRPAATKKAGQAKKKK (SEQ ID NO: 301). In some embodiments, a peptide described herein is conjugated to, linked to, or fused to a nuclear localization signal comprising KxRy (SEQ ID NO: 302), wherein x and y independently can be 0, 1, 2, 3, 4, 5, 6, 7, 8, 9, or 10, such as KKRR (SEQ ID NO: 303), KKKRR (SEQ ID NO: 304), or KKKK (SEQ ID NO: 305). Other cell penetrating moieties can also be linked to, conjugated to, linked to, or fused to the peptides described herein, including, but not limited to, polycations, polyorganic acids, endosomal releasing polymers, poly(2-propylacrylic acid), poly(2-ethylacrylic acid), or any combination thereof.

In some embodiments, the peptides of the present disclosure (e.g., histidine-containing or histidine-enriched TfR-binding peptides) can have a high TfR binding affinity at physiological pH but a significantly reduced binding affinity at lower pH levels such as endosomal pH of 5.4. In some cases, the TfR-binding peptides of the present disclosure can be optimized for improved intra-vesicular (e.g., intra-endosomal) and/or intracellular delivery function while retaining high TfR binding capabilities. In some cases, histidine scans and comparative binding experiments can be performed to develop and screen for such peptides. In addition, some peptides can comprise (e.g., conjugated to, linked to, or fused to) a motif that facilitates low-pH endosomal escape of the peptide for enhanced delivery functions (e.g., intracellular delivery of a therapeutic agent). In some embodiments, an amino acid residue in a peptide of the present disclosure is substituted with a different amino acid residue to alter a pH-dependent binding affinity to TfR. The amino acid substitution may increase a binding affinity at low pH, increase a binding affinity at high pH, decrease a binding affinity at low pH, decrease a binding affinity at high pH, or a combination thereof.

In some embodiments, peptides of the present disclosure are capable of vesicular transcytosis across a cell layer or cell barrier such as the BBB. In some embodiments, the peptides target and/or penetrate into a cell or a nucleus of a cell (e.g., a brain cell). Examples of cells or tissues that can be targeted include cells or tissues associated with a disease or condition such as cells or tissues of the CNS, brain cells, cancerous cells, and other cell types, wherein certain biological pathway can be dysregulated in said cells. Further examples of cells or tissues that can be targeted include cells or tissues of the spleen, liver, kidney, muscle, bone marrow, or skin. A cell that can be targeted with the peptides of the present disclosure can be a human cell, a mammalian cell, a human or mammalian cell line, a cancer cell line, a cell extracted from a subject, in vivo, or in vitro.

In some instances, a peptide as disclosed herein can contain only one lysine residue, or no lysine residues. In some instances, one or more or all of the lysine residues in the peptide are replaced with arginine residues. In some instances, one or more or all of the methionine residues in the peptide are replaced by leucine or isoleucine. One or more or all of the tryptophan residues in the peptide can be replaced by phenylalanine or tyrosine. In some instances, one or more or all of the asparagine residues in the peptide are replaced by glutamine. In some embodiments, one or more or all of the aspartic acid residues can be replaced by glutamic acid residues. In some instances, one or more or all of the lysine residues in the peptide are replaced by alanine or arginine. In some embodiments, the N-terminus of the peptide is blocked or protected, such as by an acetyl group or a tert-butyloxycarbonyl group. Alternately or in combination, the C-terminus of the peptide can be blocked or protected, such as by an amide group or by the formation of an ester (e.g., a butyl or a benzyl ester). In some embodiments, the peptide is modified by methylation on free amines. For example, full methylation is accomplished through the use of reductive methylation with formaldehyde and sodium cyanoborohydride.

In some embodiments, the dipeptide GS can be added as the first two N-terminal amino acids of a peptide of the disclosure, as shown in SEQ ID NO: 65-SEQ ID NO: 128, or such N-terminal dipeptide GS can be absent as shown in SEQ ID NO: 1-SEQ ID NO: 64 and SEQ ID NO: 129-SEQ ID NO: 134, or can be substituted by any other one or two amino acids. In some embodiments, the dipeptide GS is used as a linker or used to couple to a linker to form a peptide conjugate or fusion molecules such as a peptide construct. In some embodiments, the linker comprises a GₓS_{y} (SEQ ID NO: 235) peptide, wherein x and y independently are any whole number, such as 1, 2, 3, 4, 5, 6, 7, 8, 9, and 10. In some embodiments, the peptide linker comprises (GS)x (SEQ ID NO: 236), wherein x can be any whole number, such as 1, 2, 3, 4, 5, 6, 7, 8, 9, and 10. In some embodiments, the peptide linker comprises GGSSG (SEQ ID NO: 237), GGGGG (SEQ ID NO: 238), GSGSGSGS (SEQ ID NO: 239), GGGGS (SEQ ID NO: 241), GGGS (SEQ ID NO: 234), or a variant or fragment thereof.

The peptide oligonucleotide complexes of the present disclosure include TfR-binding peptide and peptide variants within the peptide oligonucleotide complex wherein the TfR-binding peptide is a fragment (e.g., a functional fragment that retains TfR-binding function) or comprises variations relative to a reference sequence. In some embodiments of the present disclosure, a peptide as described herein comprises an amino acid sequence set forth in any one of SEQ ID NO: 1-SEQ ID NO: 134 or SEQ ID NO: 306-SEQ ID NO: 335. A peptide as disclosed herein can be a fragment comprising a contiguous fragment of any one of SEQ ID NO: 1-SEQ ID NO: 134 or SEQ ID NO: 306-SEQ ID NO: 335 that is at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36 at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 residues long, wherein the peptide fragment is selected from any portion of the peptide. In some embodiments, the peptide sequence is flanked by additional amino acids. One or more additional amino acids, for example, confer a particular in vivo charge, isoelectric point, chemical conjugation site, stability, or physiologic property to a peptide.

In some embodiments, the peptide of the peptide oligonucleotide complex may comprise not more than 49, not more than 50, not more than 51, not more than 52, not more than 53, not more than 54, not more than 55, not more than 60, not more than 65, not more than 70, not more than 75, not more than 80, not more than 85, not more than 90, not more than 95, not more than 100, not more than 110, not more than 115, not more than 120, not more than 125, not more than 130, not more than 135, not more than 140, not more than 145, or not more than 150 amino acid residues.

In some instances, the peptides as described herein that are capable of targeting and binding to a TfR comprise no more than 80 amino acids in length, or no more than 70, no more than 60, no more than 40, no more than 35, no more than 30, no more than 25, no more than 20, no more than 15, or no more than 10 amino acids in length.

In other embodiments, peptides can be conjugated to, linked to, or fused to a carrier or a molecule with targeting or homing function for a cell of interest or a target cell. In other embodiments, peptides can be conjugated to, linked to, or fused to a molecule that extends half-life or modifies the pharmacodynamic and/or pharmacokinetic properties of the peptides, or any combination thereof.

The peptide oligonucleotide complexes of the present disclosure include TfR-binding peptide and peptide variants within the peptide oligonucleotide complex wherein the TfR-binding peptide portion further contains an arginine patch or a limited number of neutral or charged residues. In some instances, a peptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 positively charged residues, such as Arg or Lys, or any combination thereof. In some instances, one or more lysine residues in the peptide are replaced with arginine residues. In some embodiments, peptides comprise one or more Arg patches. In some embodiments, an Arg patch is positioned in the N-terminus of a peptide. In other aspects, an Arg patch is positioned in the C-terminus of a peptide. In some embodiments, an Arg patch comprises 8 consecutive Arg residues (SEQ ID NO: 143). In some embodiments, 1 or more, 2 or more, 3 or more, 4 or more, 5 or more, 6 or more, 7 or more, 8 or more, 9 or more, or 10 or more Arg or Lys residues are solvent exposed on a peptide. In some embodiments, an Arg patch can be two or more consecutive Arg residues. In some embodiments, an Arg patch comprises one or more Arg substituted with Lys.

The peptides of the present disclosure can further comprise neutral amino acid residues. In some embodiments, the peptide has 35 or fewer neutral amino acid residues. In other embodiments, the peptide has 81 or fewer neutral amino acid residues, 70 or fewer neutral amino acid residues, 60 or fewer neutral amino acid residues, 50 or fewer neutral amino acid residues, 40 or fewer neutral amino acid residues, 36 or fewer neutral amino acid residues, 33 or fewer neutral amino acid residues, 30 or fewer neutral amino acid residues, 25 or fewer neutral amino acid residues, or 10 or fewer neutral amino acid residues.

The peptides of the present disclosure can further comprise negative amino acid residues. In some embodiments the peptide has 6 or fewer negative amino acid residues, 5 or fewer negative amino acid residues, 4 or fewer negative amino acid residues, 3 or fewer negative amino acid residues, 2 or fewer negative amino acid residues, or 1 or fewer negative amino acid residues. While negative amino acid residues can be selected from any negatively charged amino acid residues, in some embodiments, the negative amino acid residues are either E, or D, or a combination of both E and D.

The peptide oligonucleotide complexes of the present disclosure include TfR-binding peptide and peptide variants within the peptide oligonucleotide complex wherein the TfR-binding peptide portion has a tertiary structure. A tertiary structure may enable binding to TfR and transcytosis across a cell membrane or endocytosing into a cell. In some embodiments of the present disclosure, a three-dimensional or tertiary structure of a peptide is primarily comprised of beta-sheets and/or alpha-helix structures. In some embodiments, designed or engineered TfR-binding peptides of the present disclosure are small, compact peptides or polypeptides stabilized by intra-chain disulfide bonds (e.g., mediated by cysteines) to form cystine and a hydrophobic core. In some embodiments, engineered TfR-binding peptides have structures comprising helical bundles with at least one disulfide bridge between each of the alpha helices, thereby stabilizing the peptides. In other embodiments, the engineered TfR-binding peptides comprise structures with three alpha helices and three intra-chain disulfide bonds, one between each of the three alpha helices in the bundle of alpha helices.

In other embodiments, peptides can be conjugated to, linked to, or fused to a molecule (e.g., small molecule, peptide, or protein) with targeting or homing function for a cell of interest or a target protein located on the surface or inside said cell. In other embodiments, peptides can be conjugated to, linked to, or fused to a molecule that extends the plasma and/or biological half-life, or modifies the pharmacodynamic (e.g., enhanced binding to a target protein) and/or pharmacokinetic properties (e.g., rate and mode of clearance) of the peptides, or any combination thereof.

Generally, the nuclear magnetic resonance (NMR) solution structures or X-ray crystallography structures of related structural homologs can be used to inform mutational strategies that can improve the folding, stability, and manufacturability of the peptides as described herein, while maintaining a particular biological function (e.g., binding to TfR). These techniques can be used to predict the 3D pharmacophore of a group of structurally homologous scaffolds, as wells as to predict possible graft regions of related proteins to create chimeras with improved properties (e.g., binding properties). For example, this strategy is used to identify critical amino acid positions and loops that are used to design peptides with improved TfR receptor binding and transcytosis properties, high expression, high stability in vivo, or any combination of these properties.

In some embodiments, a peptide capable of binding TfR and transcytosis across a cell membrane or endocytosis into a cell comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with any one of the exemplary peptide sequences listed in TABLE 1 (SEQ ID NO: 1-SEQ ID NO: 128) or TABLE 12 (SEQ ID NO: 129-SEQ ID NO: 134), or a functional fragment thereof. In some embodiments, a peptide capable of binding TfR and transcytosis across a cell membrane or endocytosis into a cell comprises a sequence of any one of SEQ ID NO: 306-SEQ ID NO: 335. Two or more peptides can share a degree of sequence identity or homology and share similar properties in vivo. For instance, a peptide can share a degree of sequence identity or homology with any one of the peptides of SEQ ID NO: 1-SEQ ID NO: 134. In some embodiments, one or more peptides of the present disclosure have up to about 20% pairwise sequence identity or homology, up to about 25% pairwise sequence identity or homology, up to about 30% pairwise sequence identity or homology, up to about 35% pairwise sequence identity or homology, up to about 40% pairwise sequence identity or homology, up to about 45% pairwise sequence identity or homology, up to about 50% pairwise sequence identity or homology, up to about 55% pairwise sequence identity or homology, up to about 60% pairwise sequence identity or homology, up to about 65% pairwise sequence identity or homology, up to about 70% pairwise sequence identity or homology, up to about 75% pairwise sequence identity or homology, up to about 80% pairwise sequence identity or homology, up to about 85% pairwise sequence identity or homology, up to about 90% pairwise sequence identity or homology, up to about 95% pairwise sequence identity or homology, up to about 96% pairwise sequence identity or homology, up to about 97% pairwise sequence identity or homology, up to about 98% pairwise sequence identity or homology, up to about 99% pairwise sequence identity or homology, up to about 99.5% pairwise sequence identity or homology, or up to about 99.9% pairwise sequence identity or homology. In some embodiments, one or more peptides of the disclosure have at least about 20% pairwise sequence identity or homology, at least about 25% pairwise sequence identity or homology, at least about 30% pairwise sequence identity or homology, at least about 35% pairwise sequence identity or homology, at least about 40% pairwise sequence identity or homology, at least about 45% pairwise sequence identity or homology, at least about 50% pairwise sequence identity or homology, at least about 55% pairwise sequence identity or homology, at least about 60% pairwise sequence identity or homology, at least about 65% pairwise sequence identity or homology, at least about 70% pairwise sequence identity or homology, at least about 75% pairwise sequence identity or homology, at least about 80% pairwise sequence identity or homology, at least about 85% pairwise sequence identity or homology, at least about 90% pairwise sequence identity or homology, at least about 95% pairwise sequence identity or homology, at least about 96% pairwise sequence identity or homology, at least about 97% pairwise sequence identity or homology, at least about 98% pairwise sequence identity or homology, at least about 99% pairwise sequence identity or homology, at least about 99.5% pairwise sequence identity or homology, at least about 99.9% pairwise sequence identity or homology with a second peptide.

In some embodiments, peptides that exhibit an improved TfR receptor binding show improved transcytosis function. In some cases, peptides that exhibit an improved TfR receptor binding show no or small changes in transcytosis function. In some cases, peptides that exhibit an improved TfR receptor binding show reduced transcytosis function. In some embodiments, the K_{A} and K_{D} values of a TfR-binding peptide can be modulated and optimized (e.g., via amino acid substitutions) to provide an optimal ratio of TfR-binding affinity and efficient transcytosis function.

Various methods and software programs can be used to determine the homology between two or more peptides, such as NCBI BLAST, Clustal W, MAFFT, Clustal Omega, AlignMe, Praline, or another suitable method or algorithm. Pairwise sequence alignment can be used to identify regions of similarity that can indicate functional, structural and/or evolutionary relationships between two biological sequences (e.g., amino acid or nucleic acid sequences). In addition, multiple sequence alignment (MSA) is the alignment of three or more biological sequences. From the output of MSA applications, homology can be inferred and the evolutionary relationship between the sequences assessed. As used herein, “sequence homology” and “sequence identity” and “percent (%) sequence identity” and “percent (%) sequence homology” are used interchangeably to mean the sequence relatedness or variation, as appropriate, to a reference polynucleotide or amino acid sequence.

In some instances, the peptide is any one of SEQ ID NO: 1-SEQ ID NO: 134 or SEQ ID NO: 306-SEQ ID NO: 335, or a functional fragment thereof. In other embodiments, the peptide of the disclosure further comprises a peptide with 99%, 95%, 90%, 85%, or 80% sequence identity or homology to any one of SEQ ID NO: 1-SEQ ID NO: 134 or functional fragment thereof.

In other instances, the peptide can be a peptide that is homologous to any one of SEQ ID NO: 1-SEQ ID NO: 134, or a functional fragment thereof. As further described herein, the term “homologous” can be used herein to denote peptide having at least 70%, at least 80%, at least 90%, at least 95%, or greater than 95% sequence identity or homology to a sequence of any one of SEQ ID NO: 1-SEQ ID NO: 134 or a functional fragment thereof.

In still other instances, the nucleic acid molecules that encode a peptide of any one of SEQ ID NO: 1-SEQ ID NO: 134 can be identified by either a determination of the sequence identity or homology of the encoded peptide amino acid sequence with the amino acid sequence of any one of SEQ ID NO: 1-SEQ ID NO: 134, or by a nucleic acid hybridization assay. Such peptide variants of any one of SEQ ID NO: 1-SEQ ID NO: 134 can be characterized as nucleic acid molecules (1) that remain hybridized with a nucleic acid molecule having the nucleotide sequence of any one of SEQ ID NO: 1-SEQ ID NO: 134 (or its complement) under highly stringent washing conditions, in which the wash stringency is equivalent to 0.1×-0.2×SSC with 0.10% SDS at 50-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 134.

### Knotted Peptides

The peptide oligonucleotide complexes of the present disclosure may include peptide and peptide variants within the peptide oligonucleotide complex, wherein the peptide portion comprises a knotted peptide or cystine dense peptide (CDP). In some embodiments, TfR-binding peptides of the present disclosure comprise one or more Cys, or one or more disulfide bond. In some embodiments, the TfR-binding peptides are derived from cystine-dense peptides (CDPs), knotted peptides, or hitchins. As used herein, the term “peptide” is considered to be interchangeable with the terms “knotted peptide”, “cystine-dense peptide”, “CDP”, and “hitchin”. (See e.g., Correnti et al. Screening, large-scale production, and structure-based classification for cystine-dense peptides. Nat Struct Mol Biol. 2018 March; 25(3): 270-278).

In some embodiments, the TfR-binding peptides of the present disclosure can bind TfR, thereby preventing TfR interactions such as interactions of TfR with other exogenous or endogenous ligands (e.g., Tf or homologs or fragments thereof). In some embodiments, the TfR-binding peptides of the present disclosure can bind TfR without impacting the binding of other exogenous or endogenous ligands (e.g., Tf or homologs or fragments thereof) with TfR. In some embodiments, TfR-binding peptides may be engineered peptides. An engineered peptide may be a peptide that is non-naturally occurring, artificial, isolated, synthetic, designed, or recombinantly expressed. In some embodiments, the TfR-binding peptides of the present disclosure comprise one or more properties of CDPs, knotted peptides, or hitchins, such as stability, resistance to proteolysis, resistance to reducing conditions, and/or ability to cross the blood brain barrier.

In some embodiments, CDPs or knotted peptides, including engineered, non-naturally occurring CDPs and those found in nature, can be conjugated to, linked to, or fused to the TfR-binding peptides of the present disclosure, such as those described in TABLE 1, to provide additional homing or targeting function to a target cell, such as a cancer cell, pancreatic cell, liver cell, colon cell, ovarian cell, breast cell, lung cell, or any combination thereof. In some embodiments, CDPs or knotted peptides, including engineered CDPs, isolated CDPs, and CDPs found in nature, can be conjugated to, linked to, or fused to the TfR-binding peptides of the present disclosure, such as those described in TABLE 1, to provide additional homing or targeting function to a target cell, such as a cancer cell, pancreatic cell, liver cell, colon cell, ovarian cell, breast cell, lung cell, or any combination thereof. An engineered peptide may be a peptide that is non-naturally occurring, artificial, synthetic, designed, or recombinantly expressed. In some embodiments, a TfR-binding peptide of the present disclosure enables TfR-mediated transcytosis and/or cellular endocytosis, and the additional CDP or knotted peptide that is conjugated to, linked to, or fused to TfR-binding peptide can selectively target an enzyme or other protein of interest in a cell associated with a disease or condition. In some cases, the cell is a cancer cell. Cancers can include breast cancer, liver cancer, colon cancer, brain cancer, spleen cancer, cancers of the salivary gland, kidney cancer, muscle cancers, ovarian cancer, glioblastoma, astrocytoma, glioma, medulloblastoma, ependymoma, choroid plexus carcinoma, midline glioma, diffuse intrinsic pontine glioma, lung cancer, bone marrow cell cancers, or skin cancer, genitourinary cancer, osteosarcoma, muscle-derived sarcoma, melanoma, head and neck cancer, a neuroblastoma, glioblastoma, astrocytoma, glioma, medulloblastoma, ependymoma, choroid plexus carcinoma, midline glioma, and diffuse intrinsic pontine glioma (DIPG), or a CMYC-overexpressing cancer. In some cases, other CDP or knotted peptides (e.g., those found in nature) are conjugated to, linked to, or fused to TfR-binding peptides and are capable of localizing TfR-binding peptides across the blood brain barrier to deliver TfR-binding peptides to target cells in the central nervous system.

CDPs (e.g., knotted peptides) are a class of peptides, usually ranging from about 11 to about 81 amino acids in length that are often folded into a compact structure. Knotted peptides are typically assembled into a complex tertiary structure that is characterized by a number of intramolecular disulfide crosslinks and may contain beta strands, alpha helices, and other secondary structures. The presence of the disulfide bonds gives knotted peptides remarkable environmental stability, allowing them to withstand extremes of temperature and pH and to resist the proteolytic enzymes of the blood stream. The presence of a disulfide knot may provide resistance to reduction by reducing agents. The rigidity of knotted peptides also allows them to bind to targets without paying the “entropic penalty” that a floppy peptide accrues upon binding a target. For example, binding is adversely affected by the loss of entropy that occurs when a peptide binds a target to form a complex. Therefore, “entropic penalty” is the adverse effect on binding, and the greater the entropic loss that occurs upon this binding, the greater the “entropic penalty.” Furthermore, unbound molecules that are flexible lose more entropy when forming a complex than molecules that are rigidly structured, because of the loss of flexibility when bound up in a complex. However, rigidity in the unbound molecule also generally increases specificity by limiting the number of complexes that molecule can form. The peptides can bind targets with antibody-like affinity, or with nanomolar or picomolar affinity. A wider examination of the sequence structure and sequence identity or homology of knotted peptides reveals that they have arisen by convergent evolution in all kinds of animals and plants. In animals, they are often found in venoms, for example, the venoms of spiders and scorpions and have been implicated in the modulation of ion channels. The knotted proteins of plants can inhibit the proteolytic enzymes of animals or have antimicrobial activity, suggesting that knotted peptides can function in molecular defense systems found in plants.

The peptides of the present disclosure comprise cysteine amino acid residues. In some embodiments, the peptide has at least 1, at least 2, at least 3, at least 4, at least 5, at least 6, at least 7, at least 8, at least 9, or at least 10 cysteine amino acid residues. In some embodiments, the peptide has at least 8 cysteine amino acid residues. In other embodiments, the peptide has at least 10 cysteine amino acid residues, at least 12 cysteine amino acid residues, at least 14 cysteine amino acid residues or at least 16 cysteine amino acid residues.

A knotted peptide can comprise disulfide bridges. A knotted peptide can be a peptide wherein 5% or more of the residues are cysteines forming intramolecular disulfide bonds. A disulfide-linked peptide can be a drug scaffold. In some embodiments, the disulfide bridges form a knot. A disulfide bridge can be formed between cysteine residues, for example, between cysteines 1 and 4, 2 and 5, or, 3 and 6. In some embodiments, one disulfide bridge passes through a loop formed by the other two disulfide bridges, for example, to form the knot. In other embodiments, the disulfide bridges can be formed between any two cysteine residues.

The present disclosure further includes peptide scaffolds that, e.g., can be used as a starting point for generating additional peptides. In some embodiments, these scaffolds can be derived from a variety of knotted peptides (such as CDPs or knotted peptides). In certain embodiments, CDPs (e.g., knotted peptides) are assembled into a complex tertiary structure that is characterized by a number of intramolecular disulfide crosslinks, and optionally contain beta strands and other secondary structures such as an alpha helix. For example, CDPs (e.g., knotted peptides) include, in some embodiments, small disulfide-rich proteins characterized by a disulfide through disulfide knot. This knot can be, e.g., obtained when one disulfide bridge crosses the macrocycle formed by two other disulfides and the interconnecting backbone. In some embodiments, the knotted peptides can include growth factor cysteine knots or inhibitor cysteine knots. Other possible peptide structures include peptide having two parallel helices linked by two disulfide bridges without β-sheets (e.g., hefutoxin).

Some peptides of the present disclosure can comprise at least one amino acid residue in an L configuration. A peptide can comprise at least one amino acid residue in D configuration. In some embodiments, a peptide is 15-75 amino acid residues long. In other embodiments, a peptide is 11-55 amino acid residues long. In still other embodiments, a peptide is 11-65 amino acid residues long. In further embodiments, a peptide is at least 20 amino acid residues long.

Some CDPs (e.g., knotted peptides) can be derived or isolated from a class of proteins known to be present or associated with toxins or venoms. In some cases, the peptide can be derived from toxins or venoms associated with scorpions or spiders. The peptide can be derived from venoms and toxins of spiders and scorpions of various genus and species. For example, the peptide can be derived from a venom or toxin of the Leiurus quinquestriatus hebraeus, Buthus occitanus tunetanus, Hottentotta judaicus, Mesobuthus eupeus, Buthus occitanus israelis, Hadrurus gertschi, Androctonus australis, Centruroides noxius, Heterometrus laoticus, Opistophthalmus carinatus, Haplopelma schmidti, Isometrus maculatus, Haplopelma huwenum, Haplopelma hainanum, Haplopelma schmidti, Agelenopsis aperta, Haydronyche versuta, Selenocosmia huwena, Heteropoda venatoria, Grammostola rosea, Omithoctonus huwena, Hadronyche versuta, Atrax robustus, Angelenopsis aperta, Psalmopoeus cambridgei, Hadronyche infensa, Paracoelotes luctosus, and Chilobrachys jingzhaoor another suitable genus or species of scorpion or spider. In some cases, a peptide can be derived from a Buthus martensii Karsh (scorpion) toxin.

### Sequence Identity and Homology

Percent (%) sequence identity or homology is determined by conventional methods. (See e.g., Altschul et al. (1986), Bull. Math. Bio. 48:603 (1986), and Henikoff and Henikoff (1992), Proc. Natl. Acad. Sci. USA 89:10915). Briefly, two amino acid sequences can be aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Additionally, there are several established algorithms available to align two amino acid sequences. For example, the “FASTA” similarity search algorithm of Pearson and Lipman can be a suitable protein alignment method for examining the level of sequence identity or homology shared by an amino acid sequence of a peptide disclosed herein and the amino acid sequence of a peptide variant. The FASTA algorithm is described, for example, by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 65) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. For example, illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid sequences or molecules using a ratio as disclosed above. For nucleic acid sequence comparisons, the ktup value can range between one to six, preferably from three to six, most preferably three, with other parameters set as described herein.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that can be introduced into the amino acid sequences of the present disclosure. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II.RTM. viewer and homology modeling tools; MSI, San Diego, Calif), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments, determination of structure can typically be accompanied by evaluating activity of modified molecules.

### Physicochemical Properties of Peptides

The peptide oligonucleotide complexes of the present disclosure may include TfR-binding peptide and peptide variants within the peptide oligonucleotide complex wherein the TfR-binding peptide portion has physiochemical properties as described. In some embodiments, the TfR-binding peptides of the present disclosure can comprise a wide range of physicochemical properties such as molecular size and structure, pH, isoelectric point, and overall molecular net charge. These parameters can have an effect on the peptides ability to bind TfR, promote transcytosis, and transport of cargo molecules across cell barrier such as the BBB.

A peptide of the present disclosure can comprise at least one amino acid residue in D configuration. In some embodiments, a peptide is about 5-100 amino acid residues long. In some embodiments, a peptide is about 10-90 amino acid residues long. In some embodiments, a peptide is about 15-80 amino acid residues long. In some embodiments, a peptide is about 15-75 amino acid residues long. In some embodiments, a peptide is about 15-70 amino acid residues long. In some embodiments, a peptide is about 20-65 amino acid residues long. In some embodiments, a peptide is about 20-60 amino acid residues long. In some embodiments, a peptide is about 25-55 amino acid residues long. In some embodiments, a peptide is about 25-50 amino acid residues long. In some embodiments, a peptide is about 25-40 amino acid residues long. In some embodiments, a peptide is about 11-35 amino acid residues long. In some embodiments, a peptide is about 10⁻²⁵ amino acid residues long.

In some embodiments, a peptide is at least 5 amino acid residues long. In some embodiments, a peptide is at least 10 amino acid residues long. In some embodiments, a peptide is at least 15 amino acid residues long. In some embodiments, a peptide is at least 20 amino acid residues long. In some embodiments, a peptide is at least 25 amino acid residues long. In some embodiments, a peptide is at least 30 amino acid residues long. In some embodiments, a peptide is at least 35 amino acid residues long. In some embodiments, a peptide is at least 40 amino acid residues long. In some embodiments, a peptide is at least 45 amino acid residues long. In some embodiments, a peptide is at least 50 amino acid residues long. In some embodiments, a peptide is at least 55 amino acid residues long. In some embodiments, a peptide is at least 60 amino acid residues long. In some embodiments, a peptide is at least 65 amino acid residues long. In some embodiments, a peptide is at least 70 amino acid residues long. In some embodiments, a peptide is at least 75 amino acid residues long.

In some embodiments, an amino acid sequence of a peptide as described herein comprises at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 amino acid residues.

In some embodiments of the present disclosure, a three-dimensional or tertiary structure of a peptide is primarily comprised of beta-sheets and/or alpha-helix structures. In some embodiments, designed or engineered TfR-binding peptides of the present disclosure are small, compact peptides or polypeptides stabilized by intra-chain disulfide bonds (e.g., mediated by cysteines) and a hydrophobic core. In some embodiments, engineered TfR-binding peptides have structures comprising helical bundles with at least one disulfide bridge between each of the alpha helices, thereby stabilizing the peptides. In other embodiments, the engineered TfR-binding peptides comprise structures with three alpha helices and three intra-chain disulfide bonds, one between each of the three alpha helices in the bundle of alpha helices.

At physiological pH, peptides as described herein can have an overall molecular net charge, for example, of −5, −4, −3, −2, −1, 0, +1, +2, +3, +4, or +5. When the net charge is zero, the peptide can be uncharged or zwitterionic. In some embodiments, a peptide contains one or more disulfide bonds and has a positive net charge at physiological pH where the net charge can be +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10. In some embodiments, a peptide has a negative net charge at physiological pH where the net charge can be −0.5 or less than −0.5, −1 or less than −1, −1.5 or less than −1.5, −2 or less than −2, −2.5 or less than −2.5, −3 or less than −3, −3.5 or less than −3.5, −4 or less than −4, −4.5 or less than −4.5, −5 or less than −5, −5.5 or less than −5.5, −6 or less than −6, −6.5 or less than −6.5, −7 or less than −7, −7.5 or less than −7.5, −8 or less than −8, −8.5 or less than −8.5, −9 or less than −9.5, −10 or less than −10.

In some embodiments, peptides of the present disclosure can have an isoelectric point (pI) value from 3 and 10. In other embodiments, peptides of the present disclosure can have a pI value from 4.3 and 8.9. In some embodiments, peptides of the present disclosure can have a pI value from 3-4. In some embodiments, peptides of the present disclosure can have a pI value from 3-5. In some embodiments, peptides of the present disclosure can have a pI value from 3-6. In some embodiments, peptides of the present disclosure can have a pI value from 3-7. In some embodiments, peptides of the present disclosure can have a pI value from 3-8. In some embodiments, peptides of the present disclosure can have a pI value from 3-9. In some embodiments, peptides of the present disclosure can have a pI value from 4-5. In some embodiments, peptides of the present disclosure can have a pI value from 4-6. In some embodiments, peptides of the present disclosure can have a pI value from 4-7. In some embodiments, peptides of the present disclosure can have a pI value from 4-8. In some embodiments, peptides of the present disclosure can have a pI value from 4-9. In some embodiments, peptides of the present disclosure can have a pI value from 4-10. In some embodiments, peptides of the present disclosure can have a pI value from 5-6. In some embodiments, peptides of the present disclosure can have a pI value from 5-7. In some embodiments, peptides of the present disclosure can have a pI value from 5-8. In some embodiments, peptides of the present disclosure can have a pI value from 5-9. In some embodiments, peptides of the present disclosure can have a pI value from 5-10. In some embodiments, peptides of the present disclosure can have a pI value from 6-7. In some embodiments, peptides of the present disclosure can have a pI value from 6-8. In some embodiments, peptides of the present disclosure can have a pI value from 6-9. In some embodiments, peptides of the present disclosure can have a pI value from 6-10. In some embodiments, peptides of the present disclosure can have a pI value from 7-8. In some embodiments, peptides of the present disclosure can have a pI value from 7-9. In some embodiments, peptides of the present disclosure can have a pI value from 7-10. In some embodiments, peptides of the present disclosure can have a pI value from 8-9. In some embodiments, peptides of the present disclosure can have a pI value from 8-10. In some embodiments, peptides of the present disclosure can have a pI value from 9-10.

In some cases, the engineering of one or more mutations within a peptide of the present disclosure (e.g., a TfR-binding peptide) yields a peptide with an altered isoelectric point, charge, surface charge, or rheology at physiological pH. Such engineering of a mutation to a peptide that can be derived from a scorpion or spider complex can change the net charge of the peptide, for example, by decreasing the net charge by 1, 2, 3, 4, or 5, or by increasing the net charge by 1, 2, 3, 4, or 5. In such cases, the engineered mutation can facilitate the ability of the peptide to bind a target protein, promote transcytosis, and penetrate a cell, an endosome, or the nucleus. Suitable amino acid modifications for improving the rheology and potency of a peptide can include conservative or non-conservative mutations.

A peptide can comprise at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to the sequence of the venom or toxin component that the peptide is derived from. In other embodiments, a peptide, or a functional fragment thereof, comprises at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, or another suitable number as compared to the sequence of the venom or toxin component that the peptide is derived from. In some embodiments, mutations can be engineered within a peptide to provide a peptide that has a desired charge or stability at physiological pH.

Generally, the NMR solution structures, the X-ray crystal structures, as well as the primary structure sequence alignment of related structural peptide or protein homologs or in silico design can be used to generate mutational strategies that can improve the folding, stability, and/or manufacturability, while maintaining a particular biological function (e.g., TfR affinity/binding). A general strategy for producing homologs or in silico designed peptides or polypeptides can include identification of a charged surface patch or conserved residues of a protein, mutation of critical amino acid positions and loops, followed by in vitro and in vivo testing of the peptides. The overall peptide optimization process can be of iterative nature to the extent that, for example, information obtained during in vitro or in vivo testing is used for the design of the next generation of peptides. Hence, the herein disclosed methods can be used to design peptides with improved properties or to correct deleterious mutations that complicate folding and manufacturability. Key amino acid positions and loops can be retained while other residues in the peptide sequences can be mutated to improve, change, remove, or otherwise modify function, such as binding, transcytosis, or the ability to penetrate a cell, endosome, or nucleus in a cell, homing, or another activity of the peptide.

The present disclosure also encompasses multimers of the various peptides described herein. Examples of multimers include dimers, trimers, tetramers, pentamers, hexamers, heptamers, and so on. A multimer may be a homomer formed from a plurality of identical subunits or a heteromer formed from a plurality of different subunits. In some embodiments, a peptide of the present disclosure is arranged in a multimeric structure with at least one other peptide, or two, three, four, five, six, seven, eight, nine, ten, or more other peptides. In certain embodiments, the peptides of a multimeric structure each have the same sequence. In other embodiments, one or more or all of the peptides of a multimeric structure have different sequences.

In some embodiments, the present disclosure provides peptide scaffolds that can be used as a starting point for generating additional, next-generation peptides with more specific or improved properties. In some embodiments, these scaffolds are derived from a variety of CDPs or knotted peptides. Some suitable peptides for scaffolds can include, but are not limited to, chlorotoxin, brazzein, circulin, stecrisp, hanatoxin, midkine, hefutoxin, potato carboxypeptidase inhibitor, bubble protein, attractin, μ-GI, μ-GID, μ-PIIIA, ω-MVIIA, ω-CVID, χ-MrIA, ρ-TIA, conantokin G, contulakin G, GsMTx4, margatoxin, shK, toxin K, chymotrypsin inhibitor (CTI), and EGF epiregulin core. In some embodiments, the peptide sequence is flanked by additional amino acids. One or more additional amino acids can confer a desired in vivo charge, isoelectric point, chemical conjugation site, stability, or physiologic property to a peptide.

### Chemical Modification of Peptide Complexes

The peptide oligonucleotide complexes of the present disclosure may include TfR-binding peptide and peptide variants within the peptide oligonucleotide complex wherein the TfR-binding peptide portion, the oligonucleotide portion, the linker, or any other portion of the peptide complex has chemical modifications as described. A peptide oligonucleotide complex can be chemically modified one or more of a variety of ways. In some embodiments, the peptide can be mutated to add function, delete function, or modify the in vivo behavior. For example, in some embodiments, peptides of the presenting disclosure may be chemically modified with a molecule that would lead to proteasomal degradation of TfR (e.g., ubiquitin ligase engaging conjugate or fusion or cereblon-binding molecule). One or more loops between the disulfide linkages can be modified or replaced to include active elements from other peptides (such as described in Moore and Cochran, Methods in Enzymology, 503, p. 223-251, 2012). Amino acids can also be mutated, such as to increase half-life, modify, add or delete binding behavior in vivo, add new targeting function, modify surface charge and hydrophobicity, or allow conjugation sites. N-methylation is one example of methylation that can occur in a peptide of the disclosure. In some embodiments, the peptide is modified by methylation on free amines. For example, full methylation may be accomplished through the use of reductive methylation with formaldehyde and sodium cyanoborohydride.

The peptides can be modified to add function, such as to graft loops or sequences from other proteins or peptides onto peptides of this disclosure. Likewise, domains, loops, or sequences from this disclosure can be grafted onto other peptides or proteins such as antibodies that have additional function.

A chemical modification can, for instance, extend the half-life of a peptide oligonucleotide complex or change the biodistribution or pharmacokinetic profile. A chemical modification can comprise a polymer, a polyether, polyethylene glycol, a biopolymer, a polyamino acid, a fatty acid, a dendrimer, an Fc region, a simple saturated carbon chain such as palmitate or myristolate, or albumin. A polyamino acid can include, for example, a poly amino acid sequence with repeated single amino acids (e.g., poly glycine), and a poly amino acid sequence with mixed poly amino acid sequences (e.g., gly-ala-gly-ala) that may or may not follow a pattern, or any combination of the foregoing.

The peptides of the present disclosure can be modified such that the modification increases the stability and/or the half-life of the peptides. The attachment of a hydrophobic moiety, such as to the N-terminus, the C-terminus, or an internal amino acid, can be used to extend half-life of a peptide of the present disclosure. The peptide oligonucleotide complexes can also be modified to increase or decrease the gut permeability or cellular permeability of the complex. In some cases, the peptides of the present disclosure show high accumulation in glandular cells of the intestine, demonstrating applicability in the treatment and-or prevention of diseases or conditions of the intestines, such as Crohn's disease or more generally inflammatory bowel diseases. The peptide of the present disclosure can include post-translational modifications (e.g., methylation and/or amidation and/or glycosylation), which can affect, e.g., serum half-life. In some embodiments, simple carbon chains (e.g., by myristoylation and/or palmitoylation) can be conjugated to, linked to, the peptide oligonucleotide complexes. The simple carbon chains can render the fusion proteins or peptides easily separable from the unconjugated material. For example, methods that can be used to separate the fusion proteins or peptides from the unconjugated material include, but are not limited to, solvent extraction and reverse phase chromatography. Lipophilic moieties can extend half-life through reversible binding to serum albumin. Conjugated moieties can, e.g., be lipophilic moieties that extend half-life of the peptide oligonucleotide complex through reversible binding to serum albumin. In some embodiments, the lipophilic moiety can be cholesterol or a cholesterol derivative including cholestenes, cholestanes, cholestadienes and oxysterols. In some embodiments, the peptide oligonucleotide complexes can be conjugated to, linked to, myristic acid (tetradecanoic acid) or a derivative thereof. In other embodiments, the peptide oligonucleotide complexes of the present disclosure can be coupled (e.g., conjugated, linked, or fused) to a half-life modifying agent.

Examples of half-life modifying agents can include, but is not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, the half-life modifying agent may be a serum albumin binding peptide, for example SA21 (SEQ ID NO: 357, RLIEDICLPRWGCLWEDD). In some embodiments, a SA21 peptide may be conjugated or fused to the peptide oligonucleotide complexes of the present disclosure (e.g., to a TfR-binding peptide of any of SEQ ID NO: 1-SEQ ID NO: 134 or SEQ ID NO: 306-SEQ ID NO: 335). The SA21 peptide may comprise a linker sequence for conjugation to, or fusion between, one or more peptides (e.g., SEQ ID NO: 358, GGGGSGGGGSRLIEDICLPRWGCLWEDDGGGGSGGGGS). Additionally, conjugation of the peptide oligonucleotide complex to a near infrared dye, such as Cy5.5, or to an albumin binder such as Albu-tag can extend serum half-life of any peptide as described herein. In some embodiments, immunogenicity is reduced by using minimal non-human protein sequences to extend serum half-life of the peptide.

In some embodiments, the first two N-terminal amino acids (GS) of SEQ ID NO: 65-SEQ ID NO: 128 serve as a spacer or linker in order to facilitate conjugation or fusion to another molecule, such as the nucleotide of a peptide oligonucleotide complex, as well as to facilitate cleavage of the peptide from such conjugated to, linked to, or fused molecules. In some embodiments, the fusion proteins or peptides of the present disclosure can be conjugated to, linked to, or fused to other moieties that, e.g., can modify or effect changes to the properties of the peptides.

### Peptide Oligonucleotide Complexes

A peptide oligonucleotide complex, also referred to as a peptide-nucleotide agent conjugate, a peptide oligonucleotide complex, or a peptide target-binding agent complex, may comprise a peptide complexed with a nucleotide (e.g., an oligonucleotide). The peptide of the peptide oligonucleotide complex may comprise a cystine-dense peptide (CDP) (e.g., a CDP-oligonucleotide complex), as described herein. In some embodiments, the peptide may be a TfR-binding peptide (e.g., any one of SEQ ID NO: 1-SEQ ID NO: 134 or SEQ ID NO: 306-SEQ ID NO: 335). A TfR-binding peptide of a peptide oligonucleotide complex may mediate binding of the peptide oligonucleotide complex to TfR, which may facilitate transcytosis of the peptide oligonucleotide complex across a cell barrier (e.g., a BBB, a cell membrane, or a nuclear membrane). For example, a peptide oligonucleotide complex comprising a TfR-binding peptide may cross a cellular membrane, enabling interactions between the nucleotide of the peptide oligonucleotide complex and various cytosolic or nuclear components (e.g., genomic DNA, an ORF, mRNA, pre-mRNA, or DNA). In some embodiments, a peptide oligonucleotide complex comprising a TfR-binding peptide may cross a a cellular membrane by being endocytosed into a cell.

The nucleotide of the peptide oligonucleotide complex may be a target-binding agent comprising single stranded DNA, single stranded RNA, double stranded DNA, double stranded RNA, or a combination thereof. As used herein, the term “nucleotide” may refer to an oligonucleotide or polynucleotide molecule or to a single nucleotide base. For example, a nucleotide of a peptide complex may comprise a DNA or RNA oligonucleotide. In some embodiments, the nucleotide may be a small interfering RNA (siRNA), a micro RNA (miRNA, or miR), an anti-miR, an antisense RNA, an antisense oligonucleotide (ASO), a complementary RNA, a complementary DNA, an interfering RNA, a small nuclear RNA (snRNA), a spliceosomal RNA, an inhibitory RNA, a nuclear RNA, an oligonucleotide complementary to a natural antisense transcript (NAT), an aptamer, a gapmer, a splice blocker ASO, or a U1 adapter. For example, a nucleotide of the peptide oligonucleotide complex may comprise a sequence of any one of any one of SEQ ID NO: 364-SEQ ID NO: 394 or a sequence complementary to a portion of any sequence provided in TABLE 3 or an open reading frame listed in TABLE 18. In some embodiments, the nucleotide may be an siRNA that inhibits translation of a target mRNA by promoting degradation of the target mRNA. In some embodiments, the nucleotide may be an miRNA that inhibits translation of a target mRNA by promoting cleavage or destabilization of the target mRNA. In some embodiments, the nucleotide may be an aptamer that binds to a target protein, thereby inhibiting protein-protein interactions with the target protein, inhibiting enzymatic activity of the target protein, or activating the target protein.

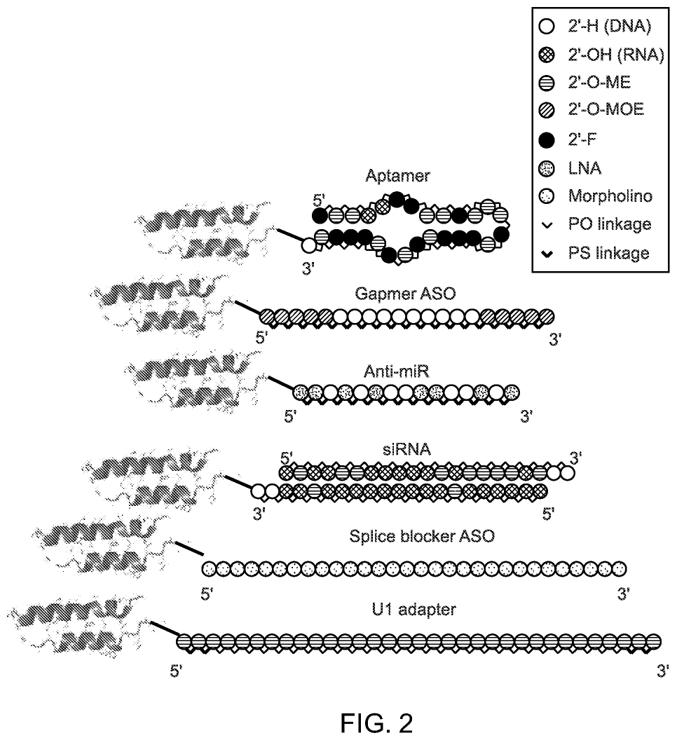

Examples of structures of various peptide oligonucleotide complexes (e.g., CDP-oligonucleotide complexes containing alternative and nonconventional bases) are illustrated in FIG. 2. Examples of APOs include an aptamer, a gapmer, an anti-miR, an siRNA, a splice blocker ASO, and a U1 adapter. The peptide portion of the peptide oligonucleotide complex (e.g., a CDP of a CDP-oligonucleotide complex) can be used to guide the nucleotide sequence (e.g., an oligonucleotide of a CDP-oligonucleotide complex) to a specific tissue, target, or cell.

In some embodiments, a peptide oligonucleotide complex binds the transferrin receptor with an affinity of no more than 10 nM, 5 nM, 1 nM, 800 pM, 600 pM, 500 pM, 400 pM, 300 PM, 250 pM, or 200 pM. In some embodiments, the affinity is identical or similar at pH 7.0 as at pH 7.4, identical or similar at pH 6.5 as at pH 7.4, identical or similar at pH 6.0 as at pH 7.4, or identical or similar at pH 5.5 as at pH 7.4. In some embodiments, the affinity is within ±1 nM, ±3 nM, ±5 nM, ±10 nM, ±30 pM, ±50 pM, ±100 pM, ±300 pM, ±500 pM, or ±1000 pM, when compared at pH 7.0 and pH 7.4, pH 6.5 and pH 7.4, pH 6.0 and pH 7.4, or pH 5.5 and pH 7.4. In some embodiments, the affinity is within 1-fold, 2-fold, 3-fold, 5-fold or 10-fold relative difference when compared at pH 7.0 and pH 7.4, pH 6.5 and pH 7.4, pH 6.0 and pH 7.4, or pH 5.5 and pH 7.4. In some aspects, the affinity of the peptide oligonucleotide complex binds the transferrin receptor is higher at a higher pH than at a lower pH. In some aspects, the higher pH is pH 7.4, pH 7.2, pH 7.0, or pH 6.8. In some aspects, the lower pH is pH 6.5, pH 6.0, pH 5.5, pH 5.0, or pH 4.5. In some aspects, the affinity of the peptide oligonucleotide complex binds the transferrin receptor is higher at pH 7.4 than at pH 6.0. In some aspects, the affinity of the peptide oligonucleotide complex binds the transferrin receptor is higher at pH 7.4 than at pH 5.5. In some aspects, the target binding peptide is capable of binding the target molecule with a dissociation constant (KD) of no more than 100 nM, no more than 20 nM, no more than 10 nM, no more than 5 nM, no more than 2 nM, no more than 1 nM, no more than 0.5 nM, no more than 0.2 nM, no more than 1 nM, or no more than 0.1 nM at pH 7.4. In some aspects, the target binding peptide is capable of binding the target molecule with a dissociation constant (KD) of no less than 1 nM, no less than 2 nM, no less than 5 nM, no less than 10 nM, no less than 20 nM, no less than 50 nM, no less than 100 nM, no less than 200 nM, or no less than 500 nM at pH 5.5. In some aspects, the affinity of the peptide oligonucleotide complex binds the transferrin receptor at pH 7.4 is at least 1.25-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, at least 100-fold, at least 500-fold, at least 1000-fold, at least 5000-fold, or at least 10,000-fold greater than the affinity of the peptide oligonucleotide complex binds the transferrin receptor at pH 5.5.

### Nucleotides

The peptide oligonucleotide complexes of the present disclosure may include nucleotide and nucleotide variants within the peptide oligonucleotide complex wherein the nucleotide portion is targeted to specific target molecule for modulation. Modulation of a target molecule may comprise degradation, inhibiting translation, decreasing expression, increasing expression, enhancing a binding interaction (e.g., a protein-protein interaction), or inhibiting a binding interaction (e.g., a protein-protein interaction). Disclosed herein are nucleotide sequences that may be used in the nucleotide portion of the peptide oligonucleotide complex, such as those targeting or complementary to nucleotides (e.g., DNA or RNA molecules) listed in TABLE 3, TABLE 18, TABLE 4, TABLE 5, TABLE 6, and EXAMPLE 50-EXAMPLE 54, or to nucleotides (e.g., DNA or RNA molecules) encoding the proteins listed in TABLE 3, TABLE 18, TABLE 4, TABLE 5, TABLE 6, and EXAMPLE 50-EXAMPLE 54, or otherwise described herein. Disclosed herein are nucleic acid sequences that may be used in the nucleotide portion of the peptide oligonucleotide complex, such as those listed in TABLE 7, and EXAMPLE 50-EXAMPLE 54. As disclosed herein, nucleic acid sequences, variants, and properties of the nucleic acids that are used in the nucleic acid portion of the peptide oligonucleotide complex may be referred to as nucleic acids of the present disclosure, nucleotides of the present disclosure, or like terminology. It may be understood that such nucleic acids or nucleotides are described in the context of the peptide oligonucleotide complexes disclosed, such as a nucleotide sequence comprising single stranded (ssDNA, ssRNA), double stranded (dsDNA, dsRNA), or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), oligonucleotide complementary to a natural antisense transcripts (NATs) sequences, siRNA, snRNA, aptamer, gapmer, anti-miR, splice blocker ASO, or U1 Adapter within the peptide oligonucleotide complex, with the accorded alterations, functions and uses described.

In some embodiments, the nucleotide sequence (e.g., a target binding agent capable of binding a target molecule) is single stranded (ssDNA, ssRNA), double stranded (dsDNA, dsRNA), or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter. Peptides according to the present disclosure can be conjugated to, linked to, or fused to such nucleotide sequences to make a peptide oligonucleotide complex. In addition, other active agents (e.g., small molecule, protein, or peptide active agents) as described herein can be conjugated to, linked to, complexed with, or fused to such nucleotide sequences, peptides or peptide oligonucleotide complex to form peptide oligonucleotide complex conjugates.

A nucleotide (e.g., a nucleotide of a peptide oligonucleotide complex) may be fully or partially reverse complementary to all or a portion of a target molecule (e.g., a target DNA or RNA sequence). In some embodiments, a target molecule expresses or encodes a protein (e.g., an mRNA encoding a protein associated with a disease). In some embodiments, a nucleotide may be fully or partially reverse complementary to a portion of an open reading frame encoding a gene or protein of interest. In some embodiments, a nucleotide may be reverse complementary to any portion of an RNA or open reading frame encoding a transcript or protein of interest. Examples of sequences that may serve as target molecules for the target binding nucleotides described herein are provided in TABLE 3 along any portion of its length. In some embodiments, a target molecule may comprise a fragment of any of the sequences provided in TABLE 18 along any portion of its length. In some embodiments, a target molecule may comprise a fragment of any of the sequences provided in TABLE 3. In some embodiments, a target molecule may comprise a sequence with one or more T residues replaced with U or one or more U residues replaced with T.

TABLE 3
Examples of Target Molecule Sequences

| SEQ | | |
|---|---|---|
| ID NO | Name | Sequence |
| SEQ | miR- | UAGCUUAUCAGACUGAUGUUGA |
| ID | 21 | |
| NO: | | |
| 395 | | |
| SEQ | IL23R | AGGCAGTGAGGAAAGAAGACATGACACAGCCAACAAGGGTGGCAGCCTGGCTC |
| ID | | TGAAGTGGAATTATGTGCTTCAAACAGGTTGAAAGAGGGAAACAGTCTTTTCCTG |
| NO: | | CTTCCAGACATGAATCAGGTCACTATTCAATGGGATGCAGTAATAGCCCTTTACA |
| 396 | | TACTCTTCAGCTGGTGTCATGGAGGAATTACAAATATAAACTGCTCTGGCCACAT |
| | | CTGGGTAGAACCAGCCACAATTTTTAAGATGGGTATGAATATCTCTATATATTGC |
| | | CAAGCAGCAATTAAGAACTGCCAACCAAGGAAACTTCATTTTTATAAAAATGGC |
| | | ATCAAAGAAAGATTTCAAATCACAAGGATTAATAAAACAACAGCTCGGCTTTGG |
| | | TATAAAAACTTTCTGGAACCACATGCTTCTATGTACTGCACTGCTGAATGTCCCA |
| | | AACATTTTCAAGAGACACTGATATGTGGAAAAGACATTTCTTCTGGATATCCGCC |
| | | AGATATTCCTGATGAAGTAACCTGTGTCATTTATGAATATTCAGGCAACATGACT |
| | | TGCACCTGGAATGCTGGGAAGCTCACCTACATAGACACAAAATACGTGGTACAT |
| | | GTGAAGAGTTTAGAGACAGAAGAAGAGCAACAGTATCTCACCTCAAGCTATATT |
| | | AACATCTCCACTGATTCATTACAAGGTGGCAAGAAGTACTTGGTTTGGGTCCAAG |
| | | CAGCAAACGCACTAGGCATGGAAGAGTCAAAACAACTGCAAATTCACCTGGATG |
| | | ATATAGTGATACCTTCTGCAGCCGTCATTTCCAGGGCTGAGACTATAAATGCTAC |
| | | AGTGCCCAAGACCATAATTTATTGGGATAGTCAAACAACAATTGAAAAGGTTTC |
| | | CTGTGAAATGAGATACAAGGCTACAACAAACCAAACTTGGAATGTTAAAGAATT |
| | | TGACACCAATTTTACATATGTGCAACAGTCAGAATTCTACTTGGAGCCAAACATT |
| | | AAGTACGTATTTCAAGTGAGATGTCAAGAAACAGGCAAAAGGTACTGGCAGCCT |
| | | TGGAGTTCACTGTTTTTTCATAAAACACCTGAAACAGTTCCCCAGGTCACATCAA |
| | | AAGCATTCCAACATGACACATGGAATTCTGGGCTAACAGTTGCTTCCATCTCTAC |
| | | AGGGCACCTTACTTCTGACAACAGAGGAGACATTGGACTTTTATTGGGAATGATC |
| | | GTCTTTGCTGTTATGTTGTCAATTCTTTCTTTGATTGGGATATTTAACAGATCATT |
| | | CCGAACTGGGATTAAAAGAAGGATCTTATTGTTAATACCAAAGTGGCTTTATGAA |
| | | GATATTCCTAATATGAAAAACAGCAATGTTGTGAAAATGCTACAGGAAAATAGT |
| | | GAACTTATGAATAATAATTCCAGTGAGCAGGTCCTATATGTTGATCCCATGATTA |
| | | CAGAGATAAAAGAAATCTTCATCCCAGAACACAAGCCTACAGACTACAAGAAGG |
| | | AGAATACAGGACCCCTGGAGACAAGAGACTACCCGCAAAACTCGCTATTCGACA |
| | | ATACTACAGTTGTATATATTCCTGATCTCAACACTGGATATAAACCCCAAATTTC |
| | | AAATTTTCTGCCTGAGGGAAGCCATCTCAGCAATAATAATGAAATTACTTCCTTA |
| | | ACACTTAAACCACCAGTTGATTCCTTAGACTCAGGAAATAATCCCAGGTTACAAA |
| | | AGCATCCTAATTTTGCTTTTTCTGTTTCAAGTGTGAATTCACTAAGCAACACAATA |
| | | TTTCTTGGAGAATTAAGCCTCATATTAAATCAAGGAGAATGCAGTTCTCCTGACA |
| | | TACAAAACTCAGTAGAGGAGGAAACCACCATGCTTTTGGAAAATGATTCACCCA |
| | | GTGAAACTATTCCAGAACAGACCCTGCTTCCTGATGAATTTGTCTCCTGTTTGGG |
| | | GATCGTGAATGAGGAGTTGCCATCTATTAATACTTATTTTCCACAAAATATTTTG |
| | | GAAAGCCACTTCAATAGGATTTCACTCTTGGAAAAGTAGAGCTGTGTGGTCAAA |
| | | ATCAATATGAGAAAGCTGCCTTGCAATCTGAACTTGGGTTTTCCCTGCAATAGAA |
| | | ATTGAATTCTGCCTCTTTTTGAAAAAAATGTATTCACATACAAATCTTCACATGG |
| | | ACACATGTTTTCATTTCCCTTGGATAAATACCTAGGTAGGGGATTGCTGGGCCAT |
| | | ATGATAAGCATATGTTTCAGTTCTACCAATCTTGTTTCCAGAGTAGTGACATTTCT |
| | | GTGCTCCTACCATCACCATGTAAGAATTCCCGGGAGCTCCATGCCTTTTTAATTTT |
| | | AGCCATTCTTCTGCCTCATTTCTTAAAATTAGAGAATTAAGGTCCCGAAGGTGGA |
| | | ACATGCTTCATGGTCACACATACAGGCACAAAAACAGCATTATGTGGACGCCTC |
| | | ATGTATTTTTTATAGAGTCAACTATTTCCTCTTTATTTTCCCTCATTGAAAGATGC |
| | | AAAACAGCTCTCTATTGTGTACAGAAAGGGTAAATAATGCAAAATACCTGGTAG |
| | | TAAAATAAATGCTGAAAATTTTCCTTTAAAATAGAATCATTAGGCCAGGCGTGGT |
| | | GGCTCATGCTTGTAATCCCAGCACTTTGGTAGGCTGAGGTAGGTGGATCACCTGA |
| | | GGTCAGGAGTTCGAGTCCAGCCTGGCCAATATGCTGAAACCCTGTCTCTACTAAA |
| | | ATTACAAAAATTAGCCGGCCATGGTGGCAGGTGCTTGTAATCCCAGCTACTTGGG |
| | | AGGCTGAGGCAGGAGAATCACTTGAACCAGGAAGGCAGAGGTTGCACTGAGCTG |
| | | AGATTGTGCCACTGCACTCCAGCCTGGGCAACAAGAGCAAAACTCTGTCTGGAA |
| | | AAAAAAAAAAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 397 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGAATCTCCCCTGCAGACCCCCACTGAGGACGGATCTGAGGAACC |
| | | GGGCTCTGAAACCTCTGATGCTAAGAGCACTCCAACAGCGGAAGATGTGACAGC |
| | | ACCCTTAGTGGATGAGGGAGCTCCCGGCAAGCAGGCTGCCGCGCAGCCCCACAC |
| | | GGAGATCCCAGAAGGAACCACAGCTGAAGAAGCAGGCATTGGAGACACCCCCA |
| | | GCCTGGAAGACGAAGCTGCTGGTCACGTGACCCAAGAGCCTGAAAGTGGTAAGG |
| | | TGGTCCAGGAAGGCTTCCTCCGAGAGCCAGGCCCCCCAGGTCTGAGCCACCAGC |
| | | TCATGTCCGGCATGCCTGGGGCTCCCCTCCTGCCTGAGGGCCCCAGAGAGGCCAC |
| | | ACGCCAACCTTCGGGGACAGGACCTGAGGACACAGAGGGCGGCCGCCACGCCCC |
| | | TGAGCTGCTCAAGCACCAGCTTCTAGGAGACCTGCACCAGGAGGGGCCGCCGCT |
| | | GAAGGGGGCAGGGGGCAAAGAGAGGCCGGGGAGCAAGGAGGAGGTGGATGAA |
| | | GACCGCGACGTCGATGAGTCCTCCCCCCAAGACTCCCCTCCCTCCAAGGCCTCCC |
| | | CAGCCCAAGATGGGCGGCCTCCCCAGACAGCCGCCAGAGAAGCCACCAGCATCC |
| | | CAGGCTTCCCAGCGGAGGGTGCCATCCCCCTCCCTGTGGATTTCCTCTCCAAAGT |
| | | TTCCACAGAGATCCCAGCCTCAGAGCCCGACGGGCCCAGTGTAGGGCGGGCCAA |
| | | AGGGCAGGATGCCCCCCTGGAGTTCACGTTTCACGTGGAAATCACACCCAACGT |
| | | GCAGAAGGAGCAGGCGCACTCGGAGGAGCATTTGGGAAGGGCTGCATTTCCAGG |
| | | GGCCCCTGGAGAGGGGCCAGAGGCCCGGGGCCCCTCTTTGGGAGAGGACACAAA |
| | | AGAGGCTGACCTTCCAGAGCCCTCTGAAAAGCAGCCTGCTGCTGCTCCGCGGGG |
| | | GAAGCCCGTCAGCCGGGTCCCTCAACTCAAAGCTCGCATGGTCAGTAAAAGCAA |
| | | AGACGGGACTGGAAGCGATGACAAAAAAGCCAAGACATCCACACGTTCCTCTGC |
| | | TAAAACCTTGAAAAATAGGCCTTGCCTTAGCCCCAAACACCCCACTCCTGGTAGC |
| | | TCAGACCCTCTGATCCAACCCTCCAGCCCTGCTGTGTGCCCAGAGCCACCTTCCT |
| | | CTCCTAAATACGTCTCTTCTGTCACTTCCCGAACTGGCAGTTCTGGAGCAAAGGA |
| | | GATGAAACTCAAGGGGGCTGATGGTAAAACGAAGATCGCCACACCGCGGGGAG |
| | | CAGCCCCTCCAGGCCAGAAGGGCCAGGCCAACGCCACCAGGATTCCAGCAAAAA |
| | | CCCCGCCCGCTCCAAAGACACCACCCAGCTCTGGTGAACCTCCAAAATCAGGGG |
| | | ATCGCAGCGGCTACAGCAGCCCCGGCTCCCCAGGCACTCCCGGCAGCCGCTCCC |
| | | GCACCCCGTCCCTTCCAACCCCACCCACCCGGGAGCCCAAGAAGGTGGCAGTGG |
| | | TCCGTACTCCACCCAAGTCGCCGTCTTCCGCCAAGAGCCGCCTGCAGACAGCCCC |
| | | CGTGCCCATGCCAGACCTGAAGAATGTCAAGTCCAAGATCGGCTCCACTGAGAA |
| | | CCTGAAGCACCAGCCGGGAGGCGGGAAGGTGCAGATAATTAATAAGAAGCTGG |
| | | ATCTTAGCAACGTCCAGTCCAAGTGTGGCTCAAAGGATAATATCAAACACGTCCC |
| | | GGGAGGCGGCAGTGTGCAAATAGTCTACAAACCAGTTGACCTGAGCAAGGTGAC |
| | | CTCCAAGTGTGGCTCATTAGGCAACATCCATCATAAACCAGGAGGTGGCCAGGT |
| | | GGAAGTAAAATCTGAGAAGCTTGACTTCAAGGACAGAGTCCAGTCGAAGATTGG |
| | | GTCCCTGGACAATATCACCCACGTCCCTGGCGGAGGAAATAAAAAGATTGAAAC |
| | | CCACAAGCTGACCTTCCGCGAGAACGCCAAAGCCAAGACAGACCACGGGGCGG |
| | | AGATCGTGTACAAGTCGCCAGTGGTGTCTGGGGACACGTCTCCACGGCATCTCAG |
| | | CAATGTCTCCTCCACCGGCAGCATCGACATGGTAGACTCGCCCCAGCTCGCCACG |
| | | CTAGCTGACGAGGTGTCTGCCTCCCTGGCCAAGCAGGGTTTGTGATCAGGCCCCT |
| | | GGGGCGGTCAATAATTGTGGAGAGGAGAGAATGAGAGAGTGTGGAAAAAAAAA |
| | | GAATAATGACCCGGCCCCCGCCCTCTGCCCCCAGCTGCTCCTCGCAGTTCGGTTA |
| | | ATTGGTTAATCACTTAACCTGCTTTTGTCACTCGGCTTTGGCTCGGGACTTCAAAA |
| | | TCAGTGATGGGAGTAAGAGCAAATTTCATCTTTCCAAATTGATGGGTGGGCTAGT |
| | | AATAAAATATTTAAAAAAAAACATTCAAAAACATGGCCACATCCAACATTTCCT |
| | | CAGGCAATTCCTTTTGATTCTTTTTTCTTCCCCCTCCATGTAGAAGAGGGAGAAG |
| | | GAGAGGCTCTGAAAGCTGCTTCTGGGGGATTTCAAGGGACTGGGGGTGCCAACC |
| | | ACCTCTGGCCCTGTTGTGGGGGTGTCACAGAGGCAGTGGCAGCAACAAAGGATT |
| | | TGAAACTTGGTGTGTTCGTGGAGCCACAGGCAGACGATGTCAACCTTGTGTGAGT |
| | | GTGACGGGGGTTGGGGTGGGGGGGGAGGCCACGGGGGAGGCCGAGGCAGGGGC |
| | | TGGGCAGAGGGGAGAGGAAGCACAAGAAGTGGGAGTGGGAGAGGAAGCCACGT |
| | | GCTGGAGAGTAGACATCCCCCTCCTTGCCGCTGGGAGAGCCAAGGCCTATGCCA |
| | | CCTGCAGCGTCTGAGCGGCCGCCTGTCCTTGGTGGCCGGGGGTGGGGGCCTGCTG |
| | | TGGGTCAGTGTGCCACCCTCTGCAGGGCAGCCTGTGGGAGAAGGGACAGCGGGT |
| | | AAAAAGAGAAGGCAAGCTGGCAGGAGGGTGGCACTTCGTGGATGACCTCCTTAG |
| | | AAAAGACTGACCTTGATGTCTTGAGAGCGCTGGCCTCTTCCTCCCTCCCTGCAGG |
| | | GTAGGGGGCCTGAGTTGAGGGGCTTCCCTCTGCTCCACAGAAACCCTGTTTTATT |
| | | GAGTTCTGAAGGTTGGAACTGCTGCCATGATTTTGGCCACTTTGCAGACCTGGGA |
| | | CTTTAGGGCTAACCAGTTCTCTTTGTAAGGACTTGTGCCTCTTGGGAGACGTCCA |
| | | CCCGTTTCCAAGCCTGGGCCACTGGCATCTCTGGAGTGTGTGGGGGTCTGGGAGG |
| | | CAGGTCCCGAGCCCCCTGTCCTTCCCACGGCCACTGCAGTCACCCCGTCTGCGCC |
| | | GCTGTGCTGTTGTCTGCCGTGAGAGCCCAATCACTGCCTATACCCCTCATCACAC |
| | | GTCACAATGTCCCGAATTCCCAGCCTCACCACCCCTTCTCAGTAATGACCCTGGT |
| | | TGGTTGCAGGAGGTACCTACTCCATACTGAGGGTGAAATTAAGGGAAGGCAAAG |
| | | TCCAGGCACAAGAGTGGGACCCCAGCCTCTCACTCTCAGTTCCACTCATCCAACT |
| | | GGGACCCTCACCACGAATCTCATGATCTGATTCGGTTCCCTGTCTCCTCCTCCCGT |
| | | CACAGATGTGAGCCAGGGCACTGCTCAGCTGTGACCCTAGGTGTTTCTGCCTTGT |
| | | TGACATGGAGAGAGCCCTTTCCCCTGAGAAGGCCTGGCCCCTTCCTGTGCTGAGC |
| | | CCACAGCAGCAGGCTGGGTGTCTTGGTTGTCAGTGGTGGCACCAGGATGGAAGG |
| | | GCAAGGCACCCAGGGCAGGCCCACAGTCCCGCTGTCCCCCACTTGCACCCTAGCT |
| | | TGTAGCTGCCAACCTCCCAGACAGCCCAGCCCGCTGCTCAGCTCCACATGCATAG |
| | | TATCAGCCCTCCACACCCGACAAAGGGGAACACACCCCCTTGGAAATGGTTCTTT |
| | | TCCCCCAGTCCCAGCTGGAAGCCATGCTGTCTGTTCTGCTGGAGCAGCTGAACAT |
| | | ATACATAGATGTTGCCCTGCCCTCCCCATCTGCACCCTGTTGAGTTGTAGTTGGAT |
| | | TTGTCTGTTTATGCTTGGATTCACCAGAGTGACTATGATAGTGAAAAGAAAAAAA |
| | | AAAAAAAAAAAGGACGCATGTATCTTGAAATGCTTGTAAAGAGGTTTCTAACCC |
| | | ACCCTCACGAGGTGTCTCTCACCCCCACACTGGGACTCGTGTGGCCTGTGTGGTG |
| | | CCACCCTGCTGGGGCCTCCCAAGTTTTGAAAGGCTTTCCTCAGCACCTGGGACCC |
| | | AACAGAGACCAGCTTCTAGCAGCTAAGGAGGCCGTTCAGCTGTGACGAAGGCCT |
| | | GAAGCACAGGATTAGGACTGAAGCGATGATGTCCCCTTCCCTACTTCCCCTTGGG |
| | | GCTCCCTGTGTCAGGGCACAGACTAGGTCTTGTGGCTGGTCTGGCTTGCGGCGCG |
| | | AGGATGGTTCTCTCTGGTCATAGCCCGAAGTCTCATGGCAGTCCCAAAGGAGGCT |
| | | TACAACTCCTGCATCACAAGAAAAAGGAAGCCACTGCCAGCTGGGGGGATCTGC |
| | | AGCTCCCAGAAGCTCCGTGAGCCTCAGCCACCCCTCAGACTGGGTTCCTCTCCAA |
| | | GCTCGCCCTCTGGAGGGGCAGCGCAGCCTCCCACCAAGGGCCCTGCGACCACAG |
| | | CAGGGATTGGGATGAATTGCCTGTCCTGGATCTGCTCTAGAGGCCCAAGCTGCCT |
| | | GCCTGAGGAAGGATGACTTGACAAGTCAGGAGACACTGTTCCCAAAGCCTTGAC |
| | | CAGAGCACCTCAGCCCGCTGACCTTGCACAAACTCCATCTGCTGCCATGAGAAA |
| | | AGGGAAGCCGCCTTTGCAAAACATTGCTGCCTAAAGAAACTCAGCAGCCTCAGG |
| | | CCCAATTCTGCCACTTCTGGTTTGGGTACAGTTAAAGGCAACCCTGAGGGACTTG |
| | | GCAGTAGAAATCCAGGGCCTCCCCTGGGGCTGGCAGCTTCGTGTGCAGCTAGAG |
| | | CTTTACCTGAAAGGAAGTCTCTGGGCCCAGAACTCTCCACCAAGAGCCTCCCTGC |
| | | CGTTCGCTGAGTCCCAGCAATTCTCCTAAGTTGAAGGGATCTGAGAAGGAGAAG |
| | | GAAATGTGGGGTAGATTTGGTGGTGGTTAGAGATATGCCCCCCTCATTACTGCCA |
| | | ACAGTTTCGGCTGCATTTCTTCACGCACCTCGGTTCCTCTTCCTGAAGTTCTTGTG |
| | | CCCTGCTCTTCAGCACCATGGGCCTTCTTATACGGAAGGCTCTGGGATCTCCCCC |
| | | TTGTGGGGCAGGCTCTTGGGGCCAGCCTAAGATCATGGTTTAGGGTGATCAGTGC |
| | | TGGCAGATAAATTGAAAAGGCACGCTGGCTTGTGATCTTAAATGAGGACAATCC |
| | | CCCCAGGGCTGGGCACTCCTCCCCTCCCCTCACTTCTCCCACCTGCAGAGCCAGT |
| | | GTCCTTGGGTGGGCTAGATAGGATATACTGTATGCCGGCTCCTTCAAGCTGCTGA |
| | | CTCACTTTATCAATAGTTCCATTTAAATTGACTTCAGTGGTGAGACTGTATCCTGT |
| | | TTGCTATTGCTTGTTGTGCTATGGGGGGAGGGGGGAGGAATGTGTAAGATAGTTA |
| | | ACATGGGCAAAGGGAGATCTTGGGGTGCAGCACTTAAACTGCCTCGTAACCCTTT |
| | | TCATGATTTCAACCACATTTGCTAGAGGGAGGGAGCAGCCACGGAGTTAGAGGC |
| | | CCTTGGGGTTTCTCTTTTCCACTGACAGGCTTTCCCAGGCAGCTGGCTAGTTCATT |
| | | CCCTCCCCAGCCAGGTGCAGGCGTAGGAATATGGACATCTGGTTGCTTTGGCCTG |
| | | CTGCCCTCTTTCAGGGGTCCTAAGCCCACAATCATGCCTCCCTAAGACCTTGGCA |
| | | TCCTTCCCTCTAAGCCGTTGGCACCTCTGTGCCACCTCTCACACTGGCTCCAGACA |
| | | CACAGCCTGTGCTTTTGGAGCTGAGATCACTCGCTTCACCCTCCTCATCTTTGTTC |
| | | TCCAAGTAAAGCCACGAGGTCGGGGCGAGGGCAGAGGTGATCACCTGCGTGTCC |
| | | CATCTACAGACCTGCAGCTTCATAAAACTTCTGATTTCTCTTCAGCTTTGAAAAG |
| | | GGTTACCCTGGGCACTGGCCTAGAGCCTCACCTCCTAATAGACTTAGCCCCATGA |
| | | GTTTGCCATGTTGAGCAGGACTATTTCTGGCACTTGCAAGTCCCATGATTTCTTCG |
| | | GTAATTCTGAGGGTGGGGGGAGGGACATGAAATCATCTTAGCTTAGCTTTCTGTC |
| | | TGTGAATGTCTATATAGTGTATTGTGTGTTTTAACAAATGATTTACACTGACTGTT |
| | | GCTGTAAAAGTGAATTTGGAAATAAAGTTATTACTCTGATTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 398 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGAATCTCCCCTGCAGACCCCCACTGAGGACGGATCTGAGGAACC |
| | | GGGCTCTGAAACCTCTGATGCTAAGAGCACTCCAACAGCGGAAGATGTGACAGC |
| | | ACCCTTAGTGGATGAGGGAGCTCCCGGCAAGCAGGCTGCCGCGCAGCCCCACAC |
| | | GGAGATCCCAGAAGGAACCACAGCTGAAGAAGCAGGCATTGGAGACACCCCCA |
| | | GCCTGGAAGACGAAGCTGCTGGTCACGTGACCCAAGCTCGCATGGTCAGTAAAA |
| | | GCAAAGACGGGACTGGAAGCGATGACAAAAAAGCCAAGGGGGCTGATGGTAAA |
| | | ACGAAGATCGCCACACCGCGGGGAGCAGCCCCTCCAGGCCAGAAGGGCCAGGC |
| | | CAACGCCACCAGGATTCCAGCAAAAACCCCGCCCGCTCCAAAGACACCACCCAG |
| | | CTCTGGTGAACCTCCAAAATCAGGGGATCGCAGCGGCTACAGCAGCCCCGGCTC |
| | | CCCAGGCACTCCCGGCAGCCGCTCCCGCACCCCGTCCCTTCCAACCCCACCCACC |
| | | CGGGAGCCCAAGAAGGTGGCAGTGGTCCGTACTCCACCCAAGTCGCCGTCTTCC |
| | | GCCAAGAGCCGCCTGCAGACAGCCCCCGTGCCCATGCCAGACCTGAAGAATGTC |
| | | AAGTCCAAGATCGGCTCCACTGAGAACCTGAAGCACCAGCCGGGAGGCGGGAA |
| | | GGTGCAGATAATTAATAAGAAGCTGGATCTTAGCAACGTCCAGTCCAAGTGTGG |
| | | CTCAAAGGATAATATCAAACACGTCCCGGGAGGCGGCAGTGTGCAAATAGTCTA |
| | | CAAACCAGTTGACCTGAGCAAGGTGACCTCCAAGTGTGGCTCATTAGGCAACAT |
| | | CCATCATAAACCAGGAGGTGGCCAGGTGGAAGTAAAATCTGAGAAGCTTGACTT |
| | | CAAGGACAGAGTCCAGTCGAAGATTGGGTCCCTGGACAATATCACCCACGTCCC |
| | | TGGCGGAGGAAATAAAAAGATTGAAACCCACAAGCTGACCTTCCGCGAGAACGC |
| | | CAAAGCCAAGACAGACCACGGGGCGGAGATCGTGTACAAGTCGCCAGTGGTGTC |
| | | TGGGGACACGTCTCCACGGCATCTCAGCAATGTCTCCTCCACCGGCAGCATCGAC |
| | | ATGGTAGACTCGCCCCAGCTCGCCACGCTAGCTGACGAGGTGTCTGCCTCCCTGG |
| | | CCAAGCAGGGTTTGTGATCAGGCCCCTGGGGCGGTCAATAATTGTGGAGAGGAG |
| | | AGAATGAGAGAGTGTGGAAAAAAAAAGAATAATGACCCGGCCCCCGCCCTCTGC |
| | | CCCCAGCTGCTCCTCGCAGTTCGGTTAATTGGTTAATCACTTAACCTGCTTTTGTC |
| | | ACTCGGCTTTGGCTCGGGACTTCAAAATCAGTGATGGGAGTAAGAGCAAATTTC |
| | | ATCTTTCCAAATTGATGGGTGGGCTAGTAATAAAATATTTAAAAAAAAACATTCA |
| | | AAAACATGGCCACATCCAACATTTCCTCAGGCAATTCCTTTTGATTCTTTTTTCTT |
| | | CCCCCTCCATGTAGAAGAGGGAGAAGGAGAGGCTCTGAAAGCTGCTTCTGGGGG |
| | | ATTTCAAGGGACTGGGGGTGCCAACCACCTCTGGCCCTGTTGTGGGGGTGTCACA |
| | | GAGGCAGTGGCAGCAACAAAGGATTTGAAACTTGGTGTGTTCGTGGAGCCACAG |
| | | GCAGACGATGTCAACCTTGTGTGAGTGTGACGGGGGTTGGGGTGGGGCGGGAGG |
| | | CCACGGGGGAGGCCGAGGCAGGGGCTGGGCAGAGGGGAGAGGAAGCACAAGA |
| | | AGTGGGAGTGGGAGAGGAAGCCACGTGCTGGAGAGTAGACATCCCCCTCCTTGC |
| | | CGCTGGGAGAGCCAAGGCCTATGCCACCTGCAGCGTCTGAGCGGCCGCCTGTCC |
| | | TTGGTGGCCGGGGGTGGGGGCCTGCTGTGGGTCAGTGTGCCACCCTCTGCAGGG |
| | | CAGCCTGTGGGAGAAGGGACAGCGGGTAAAAAGAGAAGGCAAGCTGGCAGGAG |
| | | GGTGGCACTTCGTGGATGACCTCCTTAGAAAAGACTGACCTTGATGTCTTGAGAG |
| | | CGCTGGCCTCTTCCTCCCTCCCTGCAGGGTAGGGGGCCTGAGTTGAGGGGCTTCC |
| | | CTCTGCTCCACAGAAACCCTGTTTTATTGAGTTCTGAAGGTTGGAACTGCTGCCA |
| | | TGATTTTGGCCACTTTGCAGACCTGGGACTTTAGGGCTAACCAGTTCTCTTTGTAA |
| | | GGACTTGTGCCTCTTGGGAGACGTCCACCCGTTTCCAAGCCTGGGCCACTGGCAT |
| | | CTCTGGAGTGTGTGGGGGTCTGGGAGGCAGGTCCCGAGCCCCCTGTCCTTCCCAC |
| | | GGCCACTGCAGTCACCCCGTCTGCGCCGCTGTGCTGTTGTCTGCCGTGAGAGCCC |
| | | AATCACTGCCTATACCCCTCATCACACGTCACAATGTCCCGAATTCCCAGCCTCA |
| | | CCACCCCTTCTCAGTAATGACCCTGGTTGGTTGCAGGAGGTACCTACTCCATACT |
| | | GAGGGTGAAATTAAGGGAAGGCAAAGTCCAGGCACAAGAGTGGGACCCCAGCC |
| | | TCTCACTCTCAGTTCCACTCATCCAACTGGGACCCTCACCACGAATCTCATGATCT |
| | | GATTCGGTTCCCTGTCTCCTCCTCCCGTCACAGATGTGAGCCAGGGCACTGCTCA |
| | | GCTGTGACCCTAGGTGTTTCTGCCTTGTTGACATGGAGAGAGCCCTTTCCCCTGA |
| | | GAAGGCCTGGCCCCTTCCTGTGCTGAGCCCACAGCAGCAGGCTGGGTGTCTTGGT |
| | | TGTCAGTGGTGGCACCAGGATGGAAGGGCAAGGCACCCAGGGCAGGCCCACAGT |
| | | CCCGCTGTCCCCCACTTGCACCCTAGCTTGTAGCTGCCAACCTCCCAGACAGCCC |
| | | AGCCCGCTGCTCAGCTCCACATGCATAGTATCAGCCCTCCACACCCGACAAAGG |
| | | GGAACACACCCCCTTGGAAATGGTTCTTTTCCCCCAGTCCCAGCTGGAAGCCATG |
| | | CTGTCTGTTCTGCTGGAGCAGCTGAACATATACATAGATGTTGCCCTGCCCTCCC |
| | | CATCTGCACCCTGTTGAGTTGTAGTTGGATTTGTCTGTTTATGCTTGGATTCACCA |
| | | GAGTGACTATGATAGTGAAAAGAAAAAAAAAAAAAAAAAAGGACGCATGTATC |
| | | TTGAAATGCTTGTAAAGAGGTTTCTAACCCACCCTCACGAGGTGTCTCTCACCCC |
| | | CACACTGGGACTCGTGTGGCCTGTGTGGTGCCACCCTGCTGGGGCCTCCCAAGTT |
| | | TTGAAAGGCTTTCCTCAGCACCTGGGACCCAACAGAGACCAGCTTCTAGCAGCTA |
| | | AGGAGGCCGTTCAGCTGTGACGAAGGCCTGAAGCACAGGATTAGGACTGAAGCG |
| | | ATGATGTCCCCTTCCCTACTTCCCCTTGGGGCTCCCTGTGTCAGGGCACAGACTA |
| | | GGTCTTGTGGCTGGTCTGGCTTGCGGCGCGAGGATGGTTCTCTCTGGTCATAGCC |
| | | CGAAGTCTCATGGCAGTCCCAAAGGAGGCTTACAACTCCTGCATCACAAGAAAA |
| | | AGGAAGCCACTGCCAGCTGGGGGGATCTGCAGCTCCCAGAAGCTCCGTGAGCCT |
| | | CAGCCACCCCTCAGACTGGGTTCCTCTCCAAGCTCGCCCTCTGGAGGGGCAGCGC |
| | | AGCCTCCCACCAAGGGCCCTGCGACCACAGCAGGGATTGGGATGAATTGCCTGT |
| | | CCTGGATCTGCTCTAGAGGCCCAAGCTGCCTGCCTGAGGAAGGATGACTTGACA |
| | | AGTCAGGAGACACTGTTCCCAAAGCCTTGACCAGAGCACCTCAGCCCGCTGACC |
| | | TTGCACAAACTCCATCTGCTGCCATGAGAAAAGGGAAGCCGCCTTTGCAAAACA |
| | | TTGCTGCCTAAAGAAACTCAGCAGCCTCAGGCCCAATTCTGCCACTTCTGGTTTG |
| | | GGTACAGTTAAAGGCAACCCTGAGGGACTTGGCAGTAGAAATCCAGGGCCTCCC |
| | | CTGGGGCTGGCAGCTTCGTGTGCAGCTAGAGCTTTACCTGAAAGGAAGTCTCTGG |
| | | GCCCAGAACTCTCCACCAAGAGCCTCCCTGCCGTTCGCTGAGTCCCAGCAATTCT |
| | | CCTAAGTTGAAGGGATCTGAGAAGGAGAAGGAAATGTGGGGTAGATTTGGTGGT |
| | | GGTTAGAGATATGCCCCCCTCATTACTGCCAACAGTTTCGGCTGCATTTCTTCAC |
| | | GCACCTCGGTTCCTCTTCCTGAAGTTCTTGTGCCCTGCTCTTCAGCACCATGGGCC |
| | | TTCTTATACGGAAGGCTCTGGGATCTCCCCCTTGTGGGGCAGGCTCTTGGGGCCA |
| | | GCCTAAGATCATGGTTTAGGGTGATCAGTGCTGGCAGATAAATTGAAAAGGCAC |
| | | GCTGGCTTGTGATCTTAAATGAGGACAATCCCCCCAGGGCTGGGCACTCCTCCCC |
| | | TCCCCTCACTTCTCCCACCTGCAGAGCCAGTGTCCTTGGGTGGGCTAGATAGGAT |
| | | ATACTGTATGCCGGCTCCTTCAAGCTGCTGACTCACTTTATCAATAGTTCCATTTA |
| | | AATTGACTTCAGTGGTGAGACTGTATCCTGTTTGCTATTGCTTGTTGTGCTATGGG |
| | | GGGAGGGGGGAGGAATGTGTAAGATAGTTAACATGGGCAAAGGGAGATCTTGG |
| | | GGTGCAGCACTTAAACTGCCTCGTAACCCTTTTCATGATTTCAACCACATTTGCTA |
| | | GAGGGAGGGAGCAGCCACGGAGTTAGAGGCCCTTGGGGTTTCTCTTTTCCACTG |
| | | ACAGGCTTTCCCAGGCAGCTGGCTAGTTCATTCCCTCCCCAGCCAGGTGCAGGCG |
| | | TAGGAATATGGACATCTGGTTGCTTTGGCCTGCTGCCCTCTTTCAGGGGTCCTAA |
| | | GCCCACAATCATGCCTCCCTAAGACCTTGGCATCCTTCCCTCTAAGCCGTTGGCA |
| | | CCTCTGTGCCACCTCTCACACTGGCTCCAGACACACAGCCTGTGCTTTTGGAGCT |
| | | GAGATCACTCGCTTCACCCTCCTCATCTTTGTTCTCCAAGTAAAGCCACGAGGTC |
| | | GGGGCGAGGGCAGAGGTGATCACCTGCGTGTCCCATCTACAGACCTGCAGCTTC |
| | | ATAAAACTTCTGATTTCTCTTCAGCTTTGAAAAGGGTTACCCTGGGCACTGGCCT |
| | | AGAGCCTCACCTCCTAATAGACTTAGCCCCATGAGTTTGCCATGTTGAGCAGGAC |
| | | TATTTCTGGCACTTGCAAGTCCCATGATTTCTTCGGTAATTCTGAGGGTGGGGGG |
| | | AGGGACATGAAATCATCTTAGCTTAGCTTTCTGTCTGTGAATGTCTATATAGTGT |
| | | ATTGTGTGTTTTAACAAATGATTTACACTGACTGTTGCTGTAAAAGTGAATTTGG |
| | | AAATAAAGTTATTACTCTGATTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 399 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGCTGAAGAAGCAGGCATTGGAGACACCCCCAGCCTGGAAGACGA |
| | | AGCTGCTGGTCACGTGACCCAAGCTCGCATGGTCAGTAAAAGCAAAGACGGGAC |
| | | TGGAAGCGATGACAAAAAAGCCAAGGGGGCTGATGGTAAAACGAAGATCGCCA |
| | | CACCGCGGGGAGCAGCCCCTCCAGGCCAGAAGGGCCAGGCCAACGCCACCAGG |
| | | ATTCCAGCAAAAACCCCGCCCGCTCCAAAGACACCACCCAGCTCTGGTGAACCT |
| | | CCAAAATCAGGGGATCGCAGCGGCTACAGCAGCCCCGGCTCCCCAGGCACTCCC |
| | | GGCAGCCGCTCCCGCACCCCGTCCCTTCCAACCCCACCCACCCGGGAGCCCAAG |
| | | AAGGTGGCAGTGGTCCGTACTCCACCCAAGTCGCCGTCTTCCGCCAAGAGCCGCC |
| | | TGCAGACAGCCCCCGTGCCCATGCCAGACCTGAAGAATGTCAAGTCCAAGATCG |
| | | GCTCCACTGAGAACCTGAAGCACCAGCCGGGAGGCGGGAAGGTGCAGATAATTA |
| | | ATAAGAAGCTGGATCTTAGCAACGTCCAGTCCAAGTGTGGCTCAAAGGATAATA |
| | | TCAAACACGTCCCGGGAGGCGGCAGTGTGCAAATAGTCTACAAACCAGTTGACC |
| | | TGAGCAAGGTGACCTCCAAGTGTGGCTCATTAGGCAACATCCATCATAAACCAG |
| | | GAGGTGGCCAGGTGGAAGTAAAATCTGAGAAGCTTGACTTCAAGGACAGAGTCC |
| | | AGTCGAAGATTGGGTCCCTGGACAATATCACCCACGTCCCTGGCGGAGGAAATA |
| | | AAAAGATTGAAACCCACAAGCTGACCTTCCGCGAGAACGCCAAAGCCAAGACAG |
| | | ACCACGGGGCGGAGATCGTGTACAAGTCGCCAGTGGTGTCTGGGGACACGTCTC |
| | | CACGGCATCTCAGCAATGTCTCCTCCACCGGCAGCATCGACATGGTAGACTCGCC |
| | | CCAGCTCGCCACGCTAGCTGACGAGGTGTCTGCCTCCCTGGCCAAGCAGGGTTTG |
| | | TGATCAGGCCCCTGGGGCGGTCAATAATTGTGGAGAGGAGAGAATGAGAGAGTG |
| | | TGGAAAAAAAAAGAATAATGACCCGGCCCCCGCCCTCTGCCCCCAGCTGCTCCT |
| | | CGCAGTTCGGTTAATTGGTTAATCACTTAACCTGCTTTTGTCACTCGGCTTTGGCT |
| | | CGGGACTTCAAAATCAGTGATGGGAGTAAGAGCAAATTTCATCTTTCCAAATTGA |
| | | TGGGTGGGCTAGTAATAAAATATTTAAAAAAAAACATTCAAAAACATGGCCACA |
| | | TCCAACATTTCCTCAGGCAATTCCTTTTGATTCTTTTTTCTTCCCCCTCCATGTAGA |
| | | AGAGGGAGAAGGAGAGGCTCTGAAAGCTGCTTCTGGGGGATTTCAAGGGACTGG |
| | | GGGTGCCAACCACCTCTGGCCCTGTTGTGGGGGTGTCACAGAGGCAGTGGCAGC |
| | | AACAAAGGATTTGAAACTTGGTGTGTTCGTGGAGCCACAGGCAGACGATGTCAA |
| | | CCTTGTGTGAGTGTGACGGGGGTTGGGGTGGGGCGGGAGGCCACGGGGGAGGCC |
| | | GAGGCAGGGGCTGGGCAGAGGGGAGAGGAAGCACAAGAAGTGGGAGTGGGAG |
| | | AGGAAGCCACGTGCTGGAGAGTAGACATCCCCCTCCTTGCCGCTGGGAGAGCCA |
| | | AGGCCTATGCCACCTGCAGCGTCTGAGCGGCCGCCTGTCCTTGGTGGCCGGGGGT |
| | | GGGGGCCTGCTGTGGGTCAGTGTGCCACCCTCTGCAGGGCAGCCTGTGGGAGAA |
| | | GGGACAGCGGGTAAAAAGAGAAGGCAAGCTGGCAGGAGGGTGGCACTTCGTGG |
| | | ATGACCTCCTTAGAAAAGACTGACCTTGATGTCTTGAGAGCGCTGGCCTCTTCCT |
| | | CCCTCCCTGCAGGGTAGGGGGCCTGAGTTGAGGGGCTTCCCTCTGCTCCACAGAA |
| | | ACCCTGTTTTATTGAGTTCTGAAGGTTGGAACTGCTGCCATGATTTTGGCCACTTT |
| | | GCAGACCTGGGACTTTAGGGCTAACCAGTTCTCTTTGTAAGGACTTGTGCCTCTT |
| | | GGGAGACGTCCACCCGTTTCCAAGCCTGGGCCACTGGCATCTCTGGAGTGTGTGG |
| | | GGGTCTGGGAGGCAGGTCCCGAGCCCCCTGTCCTTCCCACGGCCACTGCAGTCAC |
| | | CCCGTCTGCGCCGCTGTGCTGTTGTCTGCCGTGAGAGCCCAATCACTGCCTATAC |
| | | CCCTCATCACACGTCACAATGTCCCGAATTCCCAGCCTCACCACCCCTTCTCAGT |
| | | AATGACCCTGGTTGGTTGCAGGAGGTACCTACTCCATACTGAGGGTGAAATTAA |
| | | GGGAAGGCAAAGTCCAGGCACAAGAGTGGGACCCCAGCCTCTCACTCTCAGTTC |
| | | CACTCATCCAACTGGGACCCTCACCACGAATCTCATGATCTGATTCGGTTCCCTG |
| | | TCTCCTCCTCCCGTCACAGATGTGAGCCAGGGCACTGCTCAGCTGTGACCCTAGG |
| | | TGTTTCTGCCTTGTTGACATGGAGAGAGCCCTTTCCCCTGAGAAGGCCTGGCCCC |
| | | TTCCTGTGCTGAGCCCACAGCAGCAGGCTGGGTGTCTTGGTTGTCAGTGGTGGCA |
| | | CCAGGATGGAAGGGCAAGGCACCCAGGGCAGGCCCACAGTCCCGCTGTCCCCCA |
| | | CTTGCACCCTAGCTTGTAGCTGCCAACCTCCCAGACAGCCCAGCCCGCTGCTCAG |
| | | CTCCACATGCATAGTATCAGCCCTCCACACCCGACAAAGGGGAACACACCCCCTT |
| | | GGAAATGGTTCTTTTCCCCCAGTCCCAGCTGGAAGCCATGCTGTCTGTTCTGCTG |
| | | GAGCAGCTGAACATATACATAGATGTTGCCCTGCCCTCCCCATCTGCACCCTGTT |
| | | GAGTTGTAGTTGGATTTGTCTGTTTATGCTTGGATTCACCAGAGTGACTATGATA |
| | | GTGAAAAGAAAAAAAAAAAAAAAAAAGGACGCATGTATCTTGAAATGCTTGTA |
| | | AAGAGGTTTCTAACCCACCCTCACGAGGTGTCTCTCACCCCCACACTGGGACTCG |
| | | TGTGGCCTGTGTGGTGCCACCCTGCTGGGGCCTCCCAAGTTTTGAAAGGCTTTCC |
| | | TCAGCACCTGGGACCCAACAGAGACCAGCTTCTAGCAGCTAAGGAGGCCGTTCA |
| | | GCTGTGACGAAGGCCTGAAGCACAGGATTAGGACTGAAGCGATGATGTCCCCTT |
| | | CCCTACTTCCCCTTGGGGCTCCCTGTGTCAGGGCACAGACTAGGTCTTGTGGCTG |
| | | GTCTGGCTTGCGGCGCGAGGATGGTTCTCTCTGGTCATAGCCCGAAGTCTCATGG |
| | | CAGTCCCAAAGGAGGCTTACAACTCCTGCATCACAAGAAAAAGGAAGCCACTGC |
| | | CAGCTGGGGGGATCTGCAGCTCCCAGAAGCTCCGTGAGCCTCAGCCACCCCTCA |
| | | GACTGGGTTCCTCTCCAAGCTCGCCCTCTGGAGGGGCAGCGCAGCCTCCCACCAA |
| | | GGGCCCTGCGACCACAGCAGGGATTGGGATGAATTGCCTGTCCTGGATCTGCTCT |
| | | AGAGGCCCAAGCTGCCTGCCTGAGGAAGGATGACTTGACAAGTCAGGAGACACT |
| | | GTTCCCAAAGCCTTGACCAGAGCACCTCAGCCCGCTGACCTTGCACAAACTCCAT |
| | | CTGCTGCCATGAGAAAAGGGAAGCCGCCTTTGCAAAACATTGCTGCCTAAAGAA |
| | | ACTCAGCAGCCTCAGGCCCAATTCTGCCACTTCTGGTTTGGGTACAGTTAAAGGC |
| | | AACCCTGAGGGACTTGGCAGTAGAAATCCAGGGCCTCCCCTGGGGCTGGCAGCT |
| | | TCGTGTGCAGCTAGAGCTTTACCTGAAAGGAAGTCTCTGGGCCCAGAACTCTCCA |
| | | CCAAGAGCCTCCCTGCCGTTCGCTGAGTCCCAGCAATTCTCCTAAGTTGAAGGGA |
| | | TCTGAGAAGGAGAAGGAAATGTGGGGTAGATTTGGTGGTGGTTAGAGATATGCC |
| | | CCCCTCATTACTGCCAACAGTTTCGGCTGCATTTCTTCACGCACCTCGGTTCCTCT |
| | | TCCTGAAGTTCTTGTGCCCTGCTCTTCAGCACCATGGGCCTTCTTATACGGAAGG |
| | | CTCTGGGATCTCCCCCTTGTGGGGCAGGCTCTTGGGGCCAGCCTAAGATCATGGT |
| | | TTAGGGTGATCAGTGCTGGCAGATAAATTGAAAAGGCACGCTGGCTTGTGATCTT |
| | | AAATGAGGACAATCCCCCCAGGGCTGGGCACTCCTCCCCTCCCCTCACTTCTCCC |
| | | ACCTGCAGAGCCAGTGTCCTTGGGTGGGCTAGATAGGATATACTGTATGCCGGCT |
| | | CCTTCAAGCTGCTGACTCACTTTATCAATAGTTCCATTTAAATTGACTTCAGTGGT |
| | | GAGACTGTATCCTGTTTGCTATTGCTTGTTGTGCTATGGGGGGAGGGGGGAGGAA |
| | | TGTGTAAGATAGTTAACATGGGCAAAGGGAGATCTTGGGGTGCAGCACTTAAAC |
| | | TGCCTCGTAACCCTTTTCATGATTTCAACCACATTTGCTAGAGGGAGGGAGCAGC |
| | | CACGGAGTTAGAGGCCCTTGGGGTTTCTCTTTTCCACTGACAGGCTTTCCCAGGC |
| | | AGCTGGCTAGTTCATTCCCTCCCCAGCCAGGTGCAGGCGTAGGAATATGGACATC |
| | | TGGTTGCTTTGGCCTGCTGCCCTCTTTCAGGGGTCCTAAGCCCACAATCATGCCTC |
| | | CCTAAGACCTTGGCATCCTTCCCTCTAAGCCGTTGGCACCTCTGTGCCACCTCTCA |
| | | CACTGGCTCCAGACACACAGCCTGTGCTTTTGGAGCTGAGATCACTCGCTTCACC |
| | | CTCCTCATCTTTGTTCTCCAAGTAAAGCCACGAGGTCGGGGCGAGGGCAGAGGT |
| | | GATCACCTGCGTGTCCCATCTACAGACCTGCAGCTTCATAAAACTTCTGATTTCTC |
| | | TTCAGCTTTGAAAAGGGTTACCCTGGGCACTGGCCTAGAGCCTCACCTCCTAATA |
| | | GACTTAGCCCCATGAGTTTGCCATGTTGAGCAGGACTATTTCTGGCACTTGCAAG |
| | | TCCCATGATTTCTTCGGTAATTCTGAGGGTGGGGGGAGGGACATGAAATCATCTT |
| | | AGCTTAGCTTTCTGTCTGTGAATGTCTATATAGTGTATTGTGTGTTTTAACAAATG |
| | | ATTTACACTGACTGTTGCTGTAAAAGTGAATTTGGAAATAAAGTTATTACTCTGA |
| | | TTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 400 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGCTGAAGAAGCAGGCATTGGAGACACCCCCAGCCTGGAAGACGA |
| | | AGCTGCTGGTCACGTGACCCAAGCTCGCATGGTCAGTAAAAGCAAAGACGGGAC |
| | | TGGAAGCGATGACAAAAAAGCCAAGGGGGCTGATGGTAAAACGAAGATCGCCA |
| | | CACCGCGGGGAGCAGCCCCTCCAGGCCAGAAGGGCCAGGCCAACGCCACCAGG |
| | | ATTCCAGCAAAAACCCCGCCCGCTCCAAAGACACCACCCAGCTCTGGTGAACCT |
| | | CCAAAATCAGGGGATCGCAGCGGCTACAGCAGCCCCGGCTCCCCAGGCACTCCC |
| | | GGCAGCCGCTCCCGCACCCCGTCCCTTCCAACCCCACCCACCCGGGAGCCCAAG |
| | | AAGGTGGCAGTGGTCCGTACTCCACCCAAGTCGCCGTCTTCCGCCAAGAGCCGCC |
| | | TGCAGACAGCCCCCGTGCCCATGCCAGACCTGAAGAATGTCAAGTCCAAGATCG |
| | | GCTCCACTGAGAACCTGAAGCACCAGCCGGGAGGCGGGAAGGTGCAAATAGTCT |
| | | ACAAACCAGTTGACCTGAGCAAGGTGACCTCCAAGTGTGGCTCATTAGGCAACA |
| | | TCCATCATAAACCAGGAGGTGGCCAGGTGGAAGTAAAATCTGAGAAGCTTGACT |
| | | TCAAGGACAGAGTCCAGTCGAAGATTGGGTCCCTGGACAATATCACCCACGTCC |
| | | CTGGCGGAGGAAATAAAAAGATTGAAACCCACAAGCTGACCTTCCGCGAGAACG |
| | | CCAAAGCCAAGACAGACCACGGGGCGGAGATCGTGTACAAGTCGCCAGTGGTGT |
| | | CTGGGGACACGTCTCCACGGCATCTCAGCAATGTCTCCTCCACCGGCAGCATCGA |
| | | CATGGTAGACTCGCCCCAGCTCGCCACGCTAGCTGACGAGGTGTCTGCCTCCCTG |
| | | GCCAAGCAGGGTTTGTGATCAGGCCCCTGGGGCGGTCAATAATTGTGGAGAGGA |
| | | GAGAATGAGAGAGTGTGGAAAAAAAAAGAATAATGACCCGGCCCCCGCCCTCTG |
| | | CCCCCAGCTGCTCCTCGCAGTTCGGTTAATTGGTTAATCACTTAACCTGCTTTTGT |
| | | CACTCGGCTTTGGCTCGGGACTTCAAAATCAGTGATGGGAGTAAGAGCAAATTTC |
| | | ATCTTTCCAAATTGATGGGTGGGCTAGTAATAAAATATTTAAAAAAAAACATTCA |
| | | AAAACATGGCCACATCCAACATTTCCTCAGGCAATTCCTTTTGATTCTTTTTTCTT |
| | | CCCCCTCCATGTAGAAGAGGGAGAAGGAGAGGCTCTGAAAGCTGCTTCTGGGGG |
| | | ATTTCAAGGGACTGGGGGTGCCAACCACCTCTGGCCCTGTTGTGGGGGTGTCACA |
| | | GAGGCAGTGGCAGCAACAAAGGATTTGAAACTTGGTGTGTTCGTGGAGCCACAG |
| | | GCAGACGATGTCAACCTTGTGTGAGTGTGACGGGGGTTGGGGTGGGGCGGGAGG |
| | | CCACGGGGGAGGCCGAGGCAGGGGCTGGGCAGAGGGGAGAGGAAGCACAAGA |
| | | AGTGGGAGTGGGAGAGGAAGCCACGTGCTGGAGAGTAGACATCCCCCTCCTTGC |
| | | CGCTGGGAGAGCCAAGGCCTATGCCACCTGCAGCGTCTGAGCGGCCGCCTGTCC |
| | | TTGGTGGCCGGGGGTGGGGGCCTGCTGTGGGTCAGTGTGCCACCCTCTGCAGGG |
| | | CAGCCTGTGGGAGAAGGGACAGCGGGTAAAAAGAGAAGGCAAGCTGGCAGGAG |
| | | GGTGGCACTTCGTGGATGACCTCCTTAGAAAAGACTGACCTTGATGTCTTGAGAG |
| | | CGCTGGCCTCTTCCTCCCTCCCTGCAGGGTAGGGGGCCTGAGTTGAGGGGCTTCC |
| | | CTCTGCTCCACAGAAACCCTGTTTTATTGAGTTCTGAAGGTTGGAACTGCTGCCA |
| | | TGATTTTGGCCACTTTGCAGACCTGGGACTTTAGGGCTAACCAGTTCTCTTTGTAA |
| | | GGACTTGTGCCTCTTGGGAGACGTCCACCCGTTTCCAAGCCTGGGCCACTGGCAT |
| | | CTCTGGAGTGTGTGGGGGTCTGGGAGGCAGGTCCCGAGCCCCCTGTCCTTCCCAC |
| | | GGCCACTGCAGTCACCCCGTCTGCGCCGCTGTGCTGTTGTCTGCCGTGAGAGCCC |
| | | AATCACTGCCTATACCCCTCATCACACGTCACAATGTCCCGAATTCCCAGCCTCA |
| | | CCACCCCTTCTCAGTAATGACCCTGGTTGGTTGCAGGAGGTACCTACTCCATACT |
| | | GAGGGTGAAATTAAGGGAAGGCAAAGTCCAGGCACAAGAGTGGGACCCCAGCC |
| | | TCTCACTCTCAGTTCCACTCATCCAACTGGGACCCTCACCACGAATCTCATGATCT |
| | | GATTCGGTTCCCTGTCTCCTCCTCCCGTCACAGATGTGAGCCAGGGCACTGCTCA |
| | | GCTGTGACCCTAGGTGTTTCTGCCTTGTTGACATGGAGAGAGCCCTTTCCCCTGA |
| | | GAAGGCCTGGCCCCTTCCTGTGCTGAGCCCACAGCAGCAGGCTGGGTGTCTTGGT |
| | | TGTCAGTGGTGGCACCAGGATGGAAGGGCAAGGCACCCAGGGCAGGCCCACAGT |
| | | CCCGCTGTCCCCCACTTGCACCCTAGCTTGTAGCTGCCAACCTCCCAGACAGCCC |
| | | AGCCCGCTGCTCAGCTCCACATGCATAGTATCAGCCCTCCACACCCGACAAAGG |
| | | GGAACACACCCCCTTGGAAATGGTTCTTTTCCCCCAGTCCCAGCTGGAAGCCATG |
| | | CTGTCTGTTCTGCTGGAGCAGCTGAACATATACATAGATGTTGCCCTGCCCTCCC |
| | | CATCTGCACCCTGTTGAGTTGTAGTTGGATTTGTCTGTTTATGCTTGGATTCACCA |
| | | GAGTGACTATGATAGTGAAAAGAAAAAAAAAAAAAAAAAAGGACGCATGTATC |
| | | TTGAAATGCTTGTAAAGAGGTTTCTAACCCACCCTCACGAGGTGTCTCTCACCCC |
| | | CACACTGGGACTCGTGTGGCCTGTGTGGTGCCACCCTGCTGGGGCCTCCCAAGTT |
| | | TTGAAAGGCTTTCCTCAGCACCTGGGACCCAACAGAGACCAGCTTCTAGCAGCTA |
| | | AGGAGGCCGTTCAGCTGTGACGAAGGCCTGAAGCACAGGATTAGGACTGAAGCG |
| | | ATGATGTCCCCTTCCCTACTTCCCCTTGGGGCTCCCTGTGTCAGGGCACAGACTA |
| | | GGTCTTGTGGCTGGTCTGGCTTGCGGCGCGAGGATGGTTCTCTCTGGTCATAGCC |
| | | CGAAGTCTCATGGCAGTCCCAAAGGAGGCTTACAACTCCTGCATCACAAGAAAA |
| | | AGGAAGCCACTGCCAGCTGGGGGGATCTGCAGCTCCCAGAAGCTCCGTGAGCCT |
| | | CAGCCACCCCTCAGACTGGGTTCCTCTCCAAGCTCGCCCTCTGGAGGGGCAGCGC |
| | | AGCCTCCCACCAAGGGCCCTGCGACCACAGCAGGGATTGGGATGAATTGCCTGT |
| | | CCTGGATCTGCTCTAGAGGCCCAAGCTGCCTGCCTGAGGAAGGATGACTTGACA |
| | | AGTCAGGAGACACTGTTCCCAAAGCCTTGACCAGAGCACCTCAGCCCGCTGACC |
| | | TTGCACAAACTCCATCTGCTGCCATGAGAAAAGGGAAGCCGCCTTTGCAAAACA |
| | | TTGCTGCCTAAAGAAACTCAGCAGCCTCAGGCCCAATTCTGCCACTTCTGGTTTG |
| | | GGTACAGTTAAAGGCAACCCTGAGGGACTTGGCAGTAGAAATCCAGGGCCTCCC |
| | | CTGGGGCTGGCAGCTTCGTGTGCAGCTAGAGCTTTACCTGAAAGGAAGTCTCTGG |
| | | GCCCAGAACTCTCCACCAAGAGCCTCCCTGCCGTTCGCTGAGTCCCAGCAATTCT |
| | | CCTAAGTTGAAGGGATCTGAGAAGGAGAAGGAAATGTGGGGTAGATTTGGTGGT |
| | | GGTTAGAGATATGCCCCCCTCATTACTGCCAACAGTTTCGGCTGCATTTCTTCAC |
| | | GCACCTCGGTTCCTCTTCCTGAAGTTCTTGTGCCCTGCTCTTCAGCACCATGGGCC |
| | | TTCTTATACGGAAGGCTCTGGGATCTCCCCCTTGTGGGGCAGGCTCTTGGGGCCA |
| | | GCCTAAGATCATGGTTTAGGGTGATCAGTGCTGGCAGATAAATTGAAAAGGCAC |
| | | GCTGGCTTGTGATCTTAAATGAGGACAATCCCCCCAGGGCTGGGCACTCCTCCCC |
| | | TCCCCTCACTTCTCCCACCTGCAGAGCCAGTGTCCTTGGGTGGGCTAGATAGGAT |
| | | ATACTGTATGCCGGCTCCTTCAAGCTGCTGACTCACTTTATCAATAGTTCCATTTA |
| | | AATTGACTTCAGTGGTGAGACTGTATCCTGTTTGCTATTGCTTGTTGTGCTATGGG |
| | | GGGAGGGGGGAGGAATGTGTAAGATAGTTAACATGGGCAAAGGGAGATCTTGG |
| | | GGTGCAGCACTTAAACTGCCTCGTAACCCTTTTCATGATTTCAACCACATTTGCTA |
| | | GAGGGAGGGAGCAGCCACGGAGTTAGAGGCCCTTGGGGTTTCTCTTTTCCACTG |
| | | ACAGGCTTTCCCAGGCAGCTGGCTAGTTCATTCCCTCCCCAGCCAGGTGCAGGCG |
| | | TAGGAATATGGACATCTGGTTGCTTTGGCCTGCTGCCCTCTTTCAGGGGTCCTAA |
| | | GCCCACAATCATGCCTCCCTAAGACCTTGGCATCCTTCCCTCTAAGCCGTTGGCA |
| | | CCTCTGTGCCACCTCTCACACTGGCTCCAGACACACAGCCTGTGCTTTTGGAGCT |
| | | GAGATCACTCGCTTCACCCTCCTCATCTTTGTTCTCCAAGTAAAGCCACGAGGTC |
| | | GGGGCGAGGGCAGAGGTGATCACCTGCGTGTCCCATCTACAGACCTGCAGCTTC |
| | | ATAAAACTTCTGATTTCTCTTCAGCTTTGAAAAGGGTTACCCTGGGCACTGGCCT |
| | | AGAGCCTCACCTCCTAATAGACTTAGCCCCATGAGTTTGCCATGTTGAGCAGGAC |
| | | TATTTCTGGCACTTGCAAGTCCCATGATTTCTTCGGTAATTCTGAGGGTGGGGGG |
| | | AGGGACATGAAATCATCTTAGCTTAGCTTTCTGTCTGTGAATGTCTATATAGTGT |
| | | ATTGTGTGTTTTAACAAATGATTTACACTGACTGTTGCTGTAAAAGTGAATTTGG |
| | | AAATAAAGTTATTACTCTGATTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 401 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGAATCTCCCCTGCAGACCCCCACTGAGGACGGATCTGAGGAACC |
| | | GGGCTCTGAAACCTCTGATGCTAAGAGCACTCCAACAGCGGAAGCTGAAGAAGC |
| | | AGGCATTGGAGACACCCCCAGCCTGGAAGACGAAGCTGCTGGTCACGTGACCCA |
| | | AGCTCGCATGGTCAGTAAAAGCAAAGACGGGACTGGAAGCGATGACAAAAAAG |
| | | CCAAGGGGGCTGATGGTAAAACGAAGATCGCCACACCGCGGGGAGCAGCCCCTC |
| | | CAGGCCAGAAGGGCCAGGCCAACGCCACCAGGATTCCAGCAAAAACCCCGCCCG |
| | | CTCCAAAGACACCACCCAGCTCTGGTGAACCTCCAAAATCAGGGGATCGCAGCG |
| | | GCTACAGCAGCCCCGGCTCCCCAGGCACTCCCGGCAGCCGCTCCCGCACCCCGTC |
| | | CCTTCCAACCCCACCCACCCGGGAGCCCAAGAAGGTGGCAGTGGTCCGTACTCC |
| | | ACCCAAGTCGCCGTCTTCCGCCAAGAGCCGCCTGCAGACAGCCCCCGTGCCCATG |
| | | CCAGACCTGAAGAATGTCAAGTCCAAGATCGGCTCCACTGAGAACCTGAAGCAC |
| | | CAGCCGGGAGGCGGGAAGGTGCAGATAATTAATAAGAAGCTGGATCTTAGCAAC |
| | | GTCCAGTCCAAGTGTGGCTCAAAGGATAATATCAAACACGTCCCGGGAGGCGGC |
| | | AGTGTGCAAATAGTCTACAAACCAGTTGACCTGAGCAAGGTGACCTCCAAGTGT |
| | | GGCTCATTAGGCAACATCCATCATAAACCAGGAGGTGGCCAGGTGGAAGTAAAA |
| | | TCTGAGAAGCTTGACTTCAAGGACAGAGTCCAGTCGAAGATTGGGTCCCTGGAC |
| | | AATATCACCCACGTCCCTGGCGGAGGAAATAAAAAGATTGAAACCCACAAGCTG |
| | | ACCTTCCGCGAGAACGCCAAAGCCAAGACAGACCACGGGGCGGAGATCGTGTAC |
| | | AAGTCGCCAGTGGTGTCTGGGGACACGTCTCCACGGCATCTCAGCAATGTCTCCT |
| | | CCACCGGCAGCATCGACATGGTAGACTCGCCCCAGCTCGCCACGCTAGCTGACG |
| | | AGGTGTCTGCCTCCCTGGCCAAGCAGGGTTTGTGATCAGGCCCCTGGGGCGGTCA |
| | | ATAATTGTGGAGAGGAGAGAATGAGAGAGTGTGGAAAAAAAAAGAATAATGAC |
| | | CCGGCCCCCGCCCTCTGCCCCCAGCTGCTCCTCGCAGTTCGGTTAATTGGTTAATC |
| | | ACTTAACCTGCTTTTGTCACTCGGCTTTGGCTCGGGACTTCAAAATCAGTGATGG |
| | | GAGTAAGAGCAAATTTCATCTTTCCAAATTGATGGGTGGGCTAGTAATAAAATAT |
| | | TTAAAAAAAAACATTCAAAAACATGGCCACATCCAACATTTCCTCAGGCAATTCC |
| | | TTTTGATTCTTTTTTCTTCCCCCTCCATGTAGAAGAGGGAGAAGGAGAGGCTCTG |
| | | AAAGCTGCTTCTGGGGGATTTCAAGGGACTGGGGGTGCCAACCACCTCTGGCCCT |
| | | GTTGTGGGGGTGTCACAGAGGCAGTGGCAGCAACAAAGGATTTGAAACTTGGTG |
| | | TGTTCGTGGAGCCACAGGCAGACGATGTCAACCTTGTGTGAGTGTGACGGGGGT |
| | | TGGGGTGGGGCGGGAGGCCACGGGGGAGGCCGAGGCAGGGGCTGGGCAGAGGG |
| | | GAGAGGAAGCACAAGAAGTGGGAGTGGGAGAGGAAGCCACGTGCTGGAGAGTA |
| | | GACATCCCCCTCCTTGCCGCTGGGAGAGCCAAGGCCTATGCCACCTGCAGCGTCT |
| | | GAGCGGCCGCCTGTCCTTGGTGGCCGGGGGTGGGGGCCTGCTGTGGGTCAGTGT |
| | | GCCACCCTCTGCAGGGCAGCCTGTGGGAGAAGGGACAGCGGGTAAAAAGAGAA |
| | | GGCAAGCTGGCAGGAGGGTGGCACTTCGTGGATGACCTCCTTAGAAAAGACTGA |
| | | CCTTGATGTCTTGAGAGCGCTGGCCTCTTCCTCCCTCCCTGCAGGGTAGGGGGCC |
| | | TGAGTTGAGGGGCTTCCCTCTGCTCCACAGAAACCCTGTTTTATTGAGTTCTGAA |
| | | GGTTGGAACTGCTGCCATGATTTTGGCCACTTTGCAGACCTGGGACTTTAGGGCT |
| | | AACCAGTTCTCTTTGTAAGGACTTGTGCCTCTTGGGAGACGTCCACCCGTTTCCA |
| | | AGCCTGGGCCACTGGCATCTCTGGAGTGTGTGGGGGTCTGGGAGGCAGGTCCCG |
| | | AGCCCCCTGTCCTTCCCACGGCCACTGCAGTCACCCCGTCTGCGCCGCTGTGCTG |
| | | TTGTCTGCCGTGAGAGCCCAATCACTGCCTATACCCCTCATCACACGTCACAATG |
| | | TCCCGAATTCCCAGCCTCACCACCCCTTCTCAGTAATGACCCTGGTTGGTTGCAG |
| | | GAGGTACCTACTCCATACTGAGGGTGAAATTAAGGGAAGGCAAAGTCCAGGCAC |
| | | AAGAGTGGGACCCCAGCCTCTCACTCTCAGTTCCACTCATCCAACTGGGACCCTC |
| | | ACCACGAATCTCATGATCTGATTCGGTTCCCTGTCTCCTCCTCCCGTCACAGATGT |
| | | GAGCCAGGGCACTGCTCAGCTGTGACCCTAGGTGTTTCTGCCTTGTTGACATGGA |
| | | GAGAGCCCTTTCCCCTGAGAAGGCCTGGCCCCTTCCTGTGCTGAGCCCACAGCAG |
| | | CAGGCTGGGTGTCTTGGTTGTCAGTGGTGGCACCAGGATGGAAGGGCAAGGCAC |
| | | CCAGGGCAGGCCCACAGTCCCGCTGTCCCCCACTTGCACCCTAGCTTGTAGCTGC |
| | | CAACCTCCCAGACAGCCCAGCCCGCTGCTCAGCTCCACATGCATAGTATCAGCCC |
| | | TCCACACCCGACAAAGGGGAACACACCCCCTTGGAAATGGTTCTTTTCCCCCAGT |
| | | CCCAGCTGGAAGCCATGCTGTCTGTTCTGCTGGAGCAGCTGAACATATACATAGA |
| | | TGTTGCCCTGCCCTCCCCATCTGCACCCTGTTGAGTTGTAGTTGGATTTGTCTGTT |
| | | TATGCTTGGATTCACCAGAGTGACTATGATAGTGAAAAGAAAAAAAAAAAAAAA |
| | | AAAGGACGCATGTATCTTGAAATGCTTGTAAAGAGGTTTCTAACCCACCCTCACG |
| | | AGGTGTCTCTCACCCCCACACTGGGACTCGTGTGGCCTGTGTGGTGCCACCCTGC |
| | | TGGGGCCTCCCAAGTTTTGAAAGGCTTTCCTCAGCACCTGGGACCCAACAGAGAC |
| | | CAGCTTCTAGCAGCTAAGGAGGCCGTTCAGCTGTGACGAAGGCCTGAAGCACAG |
| | | GATTAGGACTGAAGCGATGATGTCCCCTTCCCTACTTCCCCTTGGGGCTCCCTGT |
| | | GTCAGGGCACAGACTAGGTCTTGTGGCTGGTCTGGCTTGCGGCGCGAGGATGGTT |
| | | CTCTCTGGTCATAGCCCGAAGTCTCATGGCAGTCCCAAAGGAGGCTTACAACTCC |
| | | TGCATCACAAGAAAAAGGAAGCCACTGCCAGCTGGGGGGATCTGCAGCTCCCAG |
| | | AAGCTCCGTGAGCCTCAGCCACCCCTCAGACTGGGTTCCTCTCCAAGCTCGCCCT |
| | | CTGGAGGGGCAGCGCAGCCTCCCACCAAGGGCCCTGCGACCACAGCAGGGATTG |
| | | GGATGAATTGCCTGTCCTGGATCTGCTCTAGAGGCCCAAGCTGCCTGCCTGAGGA |
| | | AGGATGACTTGACAAGTCAGGAGACACTGTTCCCAAAGCCTTGACCAGAGCACC |
| | | TCAGCCCGCTGACCTTGCACAAACTCCATCTGCTGCCATGAGAAAAGGGAAGCC |
| | | GCCTTTGCAAAACATTGCTGCCTAAAGAAACTCAGCAGCCTCAGGCCCAATTCTG |
| | | CCACTTCTGGTTTGGGTACAGTTAAAGGCAACCCTGAGGGACTTGGCAGTAGAA |
| | | ATCCAGGGCCTCCCCTGGGGCTGGCAGCTTCGTGTGCAGCTAGAGCTTTACCTGA |
| | | AAGGAAGTCTCTGGGCCCAGAACTCTCCACCAAGAGCCTCCCTGCCGTTCGCTGA |
| | | GTCCCAGCAATTCTCCTAAGTTGAAGGGATCTGAGAAGGAGAAGGAAATGTGGG |
| | | GTAGATTTGGTGGTGGTTAGAGATATGCCCCCCTCATTACTGCCAACAGTTTCGG |
| | | CTGCATTTCTTCACGCACCTCGGTTCCTCTTCCTGAAGTTCTTGTGCCCTGCTCTTC |
| | | AGCACCATGGGCCTTCTTATACGGAAGGCTCTGGGATCTCCCCCTTGTGGGGCAG |
| | | GCTCTTGGGGCCAGCCTAAGATCATGGTTTAGGGTGATCAGTGCTGGCAGATAA |
| | | ATTGAAAAGGCACGCTGGCTTGTGATCTTAAATGAGGACAATCCCCCCAGGGCT |
| | | GGGCACTCCTCCCCTCCCCTCACTTCTCCCACCTGCAGAGCCAGTGTCCTTGGGT |
| | | GGGCTAGATAGGATATACTGTATGCCGGCTCCTTCAAGCTGCTGACTCACTTTAT |
| | | CAATAGTTCCATTTAAATTGACTTCAGTGGTGAGACTGTATCCTGTTTGCTATTGC |
| | | TTGTTGTGCTATGGGGGGAGGGGGGAGGAATGTGTAAGATAGTTAACATGGGCA |
| | | AAGGGAGATCTTGGGGTGCAGCACTTAAACTGCCTCGTAACCCTTTTCATGATTT |
| | | CAACCACATTTGCTAGAGGGAGGGAGCAGCCACGGAGTTAGAGGCCCTTGGGGT |
| | | TTCTCTTTTCCACTGACAGGCTTTCCCAGGCAGCTGGCTAGTTCATTCCCTCCCCA |
| | | GCCAGGTGCAGGCGTAGGAATATGGACATCTGGTTGCTTTGGCCTGCTGCCCTCT |
| | | TTCAGGGGTCCTAAGCCCACAATCATGCCTCCCTAAGACCTTGGCATCCTTCCCT |
| | | CTAAGCCGTTGGCACCTCTGTGCCACCTCTCACACTGGCTCCAGACACACAGCCT |
| | | GTGCTTTTGGAGCTGAGATCACTCGCTTCACCCTCCTCATCTTTGTTCTCCAAGTA |
| | | AAGCCACGAGGTCGGGGCGAGGGCAGAGGTGATCACCTGCGTGTCCCATCTACA |
| | | GACCTGCAGCTTCATAAAACTTCTGATTTCTCTTCAGCTTTGAAAAGGGTTACCCT |
| | | GGGCACTGGCCTAGAGCCTCACCTCCTAATAGACTTAGCCCCATGAGTTTGCCAT |
| | | GTTGAGCAGGACTATTTCTGGCACTTGCAAGTCCCATGATTTCTTCGGTAATTCTG |
| | | AGGGTGGGGGGAGGGACATGAAATCATCTTAGCTTAGCTTTCTGTCTGTGAATGT |
| | | CTATATAGTGTATTGTGTGTTTTAACAAATGATTTACACTGACTGTTGCTGTAAAA |
| | | GTGAATTTGGAAATAAAGTTATTACTCTGATTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 402 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGAATCTCCCCTGCAGACCCCCACTGAGGACGGATCTGAGGAACC |
| | | GGGCTCTGAAACCTCTGATGCTAAGAGCACTCCAACAGCGGAAGATGTGACAGC |
| | | ACCCTTAGTGGATGAGGGAGCTCCCGGCAAGCAGGCTGCCGCGCAGCCCCACAC |
| | | GGAGATCCCAGAAGGAACCACAGCTGAAGAAGCAGGCATTGGAGACACCCCCA |
| | | GCCTGGAAGACGAAGCTGCTGGTCACGTGACCCAAGAGCCTGAAAGTGGTAAGG |
| | | TGGTCCAGGAAGGCTTCCTCCGAGAGCCAGGCCCCCCAGGTCTGAGCCACCAGC |
| | | TCATGTCCGGCATGCCTGGGGCTCCCCTCCTGCCTGAGGGCCCCAGAGAGGCCAC |
| | | ACGCCAACCTTCGGGGACAGGACCTGAGGACACAGAGGGCGGCCGCCACGCCCC |
| | | TGAGCTGCTCAAGCACCAGCTTCTAGGAGACCTGCACCAGGAGGGGCCGCCGCT |
| | | GAAGGGGGCAGGGGGCAAAGAGAGGCCGGGGAGCAAGGAGGAGGTGGATGAA |
| | | GACCGCGACGTCGATGAGTCCTCCCCCCAAGACTCCCCTCCCTCCAAGGCCTCCC |
| | | CAGCCCAAGATGGGCGGCCTCCCCAGACAGCCGCCAGAGAAGCCACCAGCATCC |
| | | CAGGCTTCCCAGCGGAGGGTGCCATCCCCCTCCCTGTGGATTTCCTCTCCAAAGT |
| | | TTCCACAGAGATCCCAGCCTCAGAGCCCGACGGGCCCAGTGTAGGGCGGGCCAA |
| | | AGGGCAGGATGCCCCCCTGGAGTTCACGTTTCACGTGGAAATCACACCCAACGT |
| | | GCAGAAGGAGCAGGCGCACTCGGAGGAGCATTTGGGAAGGGCTGCATTTCCAGG |
| | | GGCCCCTGGAGAGGGGCCAGAGGCCCGGGGCCCCTCTTTGGGAGAGGACACAAA |
| | | AGAGGCTGACCTTCCAGAGCCCTCTGAAAAGCAGCCTGCTGCTGCTCCGCGGGG |
| | | GAAGCCCGTCAGCCGGGTCCCTCAACTCAAAGCTCGCATGGTCAGTAAAAGCAA |
| | | AGACGGGACTGGAAGCGATGACAAAAAAGCCAAGACATCCACACGTTCCTCTGC |
| | | TAAAACCTTGAAAAATAGGCCTTGCCTTAGCCCCAAACACCCCACTCCTGGTAGC |
| | | TCAGACCCTCTGATCCAACCCTCCAGCCCTGCTGTGTGCCCAGAGCCACCTTCCT |
| | | CTCCTAAATACGTCTCTTCTGTCACTTCCCGAACTGGCAGTTCTGGAGCAAAGGA |
| | | GATGAAACTCAAGGGGGCTGATGGTAAAACGAAGATCGCCACACCGCGGGGAG |
| | | CAGCCCCTCCAGGCCAGAAGGGCCAGGCCAACGCCACCAGGATTCCAGCAAAAA |
| | | CCCCGCCCGCTCCAAAGACACCACCCAGCTCTGCGACTAAGCAAGTCCAGAGAA |
| | | GACCACCCCCTGCAGGGCCCAGATCTGAGAGAGGTGAACCTCCAAAATCAGGGG |
| | | ATCGCAGCGGCTACAGCAGCCCCGGCTCCCCAGGCACTCCCGGCAGCCGCTCCC |
| | | GCACCCCGTCCCTTCCAACCCCACCCACCCGGGAGCCCAAGAAGGTGGCAGTGG |
| | | TCCGTACTCCACCCAAGTCGCCGTCTTCCGCCAAGAGCCGCCTGCAGACAGCCCC |
| | | CGTGCCCATGCCAGACCTGAAGAATGTCAAGTCCAAGATCGGCTCCACTGAGAA |
| | | CCTGAAGCACCAGCCGGGAGGCGGGAAGGTGCAGATAATTAATAAGAAGCTGG |
| | | ATCTTAGCAACGTCCAGTCCAAGTGTGGCTCAAAGGATAATATCAAACACGTCCC |
| | | GGGAGGCGGCAGTGTGCAAATAGTCTACAAACCAGTTGACCTGAGCAAGGTGAC |
| | | CTCCAAGTGTGGCTCATTAGGCAACATCCATCATAAACCAGGAGGTGGCCAGGT |
| | | GGAAGTAAAATCTGAGAAGCTTGACTTCAAGGACAGAGTCCAGTCGAAGATTGG |
| | | GTCCCTGGACAATATCACCCACGTCCCTGGCGGAGGAAATAAAAAGATTGAAAC |
| | | CCACAAGCTGACCTTCCGCGAGAACGCCAAAGCCAAGACAGACCACGGGGCGG |
| | | AGATCGTGTACAAGTCGCCAGTGGTGTCTGGGGACACGTCTCCACGGCATCTCAG |
| | | CAATGTCTCCTCCACCGGCAGCATCGACATGGTAGACTCGCCCCAGCTCGCCACG |
| | | CTAGCTGACGAGGTGTCTGCCTCCCTGGCCAAGCAGGGTTTGTGATCAGGCCCCT |
| | | GGGGCGGTCAATAATTGTGGAGAGGAGAGAATGAGAGAGTGTGGAAAAAAAAA |
| | | GAATAATGACCCGGCCCCCGCCCTCTGCCCCCAGCTGCTCCTCGCAGTTCGGTTA |
| | | ATTGGTTAATCACTTAACCTGCTTTTGTCACTCGGCTTTGGCTCGGGACTTCAAAA |
| | | TCAGTGATGGGAGTAAGAGCAAATTTCATCTTTCCAAATTGATGGGTGGGCTAGT |
| | | AATAAAATATTTAAAAAAAAACATTCAAAAACATGGCCACATCCAACATTTCCT |
| | | CAGGCAATTCCTTTTGATTCTTTTTTCTTCCCCCTCCATGTAGAAGAGGGAGAAG |
| | | GAGAGGCTCTGAAAGCTGCTTCTGGGGGATTTCAAGGGACTGGGGGTGCCAACC |
| | | ACCTCTGGCCCTGTTGTGGGGGTGTCACAGAGGCAGTGGCAGCAACAAAGGATT |
| | | TGAAACTTGGTGTGTTCGTGGAGCCACAGGCAGACGATGTCAACCTTGTGTGAGT |
| | | GTGACGGGGGTTGGGGTGGGGGGGGAGGCCACGGGGGAGGCCGAGGCAGGGGC |
| | | TGGGCAGAGGGGAGAGGAAGCACAAGAAGTGGGAGTGGGAGAGGAAGCCACGT |
| | | GCTGGAGAGTAGACATCCCCCTCCTTGCCGCTGGGAGAGCCAAGGCCTATGCCA |
| | | CCTGCAGCGTCTGAGCGGCCGCCTGTCCTTGGTGGCCGGGGGTGGGGGCCTGCTG |
| | | TGGGTCAGTGTGCCACCCTCTGCAGGGCAGCCTGTGGGAGAAGGGACAGCGGGT |
| | | AAAAAGAGAAGGCAAGCTGGCAGGAGGGTGGCACTTCGTGGATGACCTCCTTAG |
| | | AAAAGACTGACCTTGATGTCTTGAGAGCGCTGGCCTCTTCCTCCCTCCCTGCAGG |
| | | GTAGGGGGCCTGAGTTGAGGGGCTTCCCTCTGCTCCACAGAAACCCTGTTTTATT |
| | | GAGTTCTGAAGGTTGGAACTGCTGCCATGATTTTGGCCACTTTGCAGACCTGGGA |
| | | CTTTAGGGCTAACCAGTTCTCTTTGTAAGGACTTGTGCCTCTTGGGAGACGTCCA |
| | | CCCGTTTCCAAGCCTGGGCCACTGGCATCTCTGGAGTGTGTGGGGGTCTGGGAGG |
| | | CAGGTCCCGAGCCCCCTGTCCTTCCCACGGCCACTGCAGTCACCCCGTCTGCGCC |
| | | GCTGTGCTGTTGTCTGCCGTGAGAGCCCAATCACTGCCTATACCCCTCATCACAC |
| | | GTCACAATGTCCCGAATTCCCAGCCTCACCACCCCTTCTCAGTAATGACCCTGGT |
| | | TGGTTGCAGGAGGTACCTACTCCATACTGAGGGTGAAATTAAGGGAAGGCAAAG |
| | | TCCAGGCACAAGAGTGGGACCCCAGCCTCTCACTCTCAGTTCCACTCATCCAACT |
| | | GGGACCCTCACCACGAATCTCATGATCTGATTCGGTTCCCTGTCTCCTCCTCCCGT |
| | | CACAGATGTGAGCCAGGGCACTGCTCAGCTGTGACCCTAGGTGTTTCTGCCTTGT |
| | | TGACATGGAGAGAGCCCTTTCCCCTGAGAAGGCCTGGCCCCTTCCTGTGCTGAGC |
| | | CCACAGCAGCAGGCTGGGTGTCTTGGTTGTCAGTGGTGGCACCAGGATGGAAGG |
| | | GCAAGGCACCCAGGGCAGGCCCACAGTCCCGCTGTCCCCCACTTGCACCCTAGCT |
| | | TGTAGCTGCCAACCTCCCAGACAGCCCAGCCCGCTGCTCAGCTCCACATGCATAG |
| | | TATCAGCCCTCCACACCCGACAAAGGGGAACACACCCCCTTGGAAATGGTTCTTT |
| | | TCCCCCAGTCCCAGCTGGAAGCCATGCTGTCTGTTCTGCTGGAGCAGCTGAACAT |
| | | ATACATAGATGTTGCCCTGCCCTCCCCATCTGCACCCTGTTGAGTTGTAGTTGGAT |
| | | TTGTCTGTTTATGCTTGGATTCACCAGAGTGACTATGATAGTGAAAAGAAAAAAA |
| | | AAAAAAAAAAAGGACGCATGTATCTTGAAATGCTTGTAAAGAGGTTTCTAACCC |
| | | ACCCTCACGAGGTGTCTCTCACCCCCACACTGGGACTCGTGTGGCCTGTGTGGTG |
| | | CCACCCTGCTGGGGCCTCCCAAGTTTTGAAAGGCTTTCCTCAGCACCTGGGACCC |
| | | AACAGAGACCAGCTTCTAGCAGCTAAGGAGGCCGTTCAGCTGTGACGAAGGCCT |
| | | GAAGCACAGGATTAGGACTGAAGCGATGATGTCCCCTTCCCTACTTCCCCTTGGG |
| | | GCTCCCTGTGTCAGGGCACAGACTAGGTCTTGTGGCTGGTCTGGCTTGCGGCGCG |
| | | AGGATGGTTCTCTCTGGTCATAGCCCGAAGTCTCATGGCAGTCCCAAAGGAGGCT |
| | | TACAACTCCTGCATCACAAGAAAAAGGAAGCCACTGCCAGCTGGGGGGATCTGC |
| | | AGCTCCCAGAAGCTCCGTGAGCCTCAGCCACCCCTCAGACTGGGTTCCTCTCCAA |
| | | GCTCGCCCTCTGGAGGGGCAGCGCAGCCTCCCACCAAGGGCCCTGCGACCACAG |
| | | CAGGGATTGGGATGAATTGCCTGTCCTGGATCTGCTCTAGAGGCCCAAGCTGCCT |
| | | GCCTGAGGAAGGATGACTTGACAAGTCAGGAGACACTGTTCCCAAAGCCTTGAC |
| | | CAGAGCACCTCAGCCCGCTGACCTTGCACAAACTCCATCTGCTGCCATGAGAAA |
| | | AGGGAAGCCGCCTTTGCAAAACATTGCTGCCTAAAGAAACTCAGCAGCCTCAGG |
| | | CCCAATTCTGCCACTTCTGGTTTGGGTACAGTTAAAGGCAACCCTGAGGGACTTG |
| | | GCAGTAGAAATCCAGGGCCTCCCCTGGGGCTGGCAGCTTCGTGTGCAGCTAGAG |
| | | CTTTACCTGAAAGGAAGTCTCTGGGCCCAGAACTCTCCACCAAGAGCCTCCCTGC |
| | | CGTTCGCTGAGTCCCAGCAATTCTCCTAAGTTGAAGGGATCTGAGAAGGAGAAG |
| | | GAAATGTGGGGTAGATTTGGTGGTGGTTAGAGATATGCCCCCCTCATTACTGCCA |
| | | ACAGTTTCGGCTGCATTTCTTCACGCACCTCGGTTCCTCTTCCTGAAGTTCTTGTG |
| | | CCCTGCTCTTCAGCACCATGGGCCTTCTTATACGGAAGGCTCTGGGATCTCCCCC |
| | | TTGTGGGGCAGGCTCTTGGGGCCAGCCTAAGATCATGGTTTAGGGTGATCAGTGC |
| | | TGGCAGATAAATTGAAAAGGCACGCTGGCTTGTGATCTTAAATGAGGACAATCC |
| | | CCCCAGGGCTGGGCACTCCTCCCCTCCCCTCACTTCTCCCACCTGCAGAGCCAGT |
| | | GTCCTTGGGTGGGCTAGATAGGATATACTGTATGCCGGCTCCTTCAAGCTGCTGA |
| | | CTCACTTTATCAATAGTTCCATTTAAATTGACTTCAGTGGTGAGACTGTATCCTGT |
| | | TTGCTATTGCTTGTTGTGCTATGGGGGGAGGGGGGAGGAATGTGTAAGATAGTTA |
| | | ACATGGGCAAAGGGAGATCTTGGGGTGCAGCACTTAAACTGCCTCGTAACCCTTT |
| | | TCATGATTTCAACCACATTTGCTAGAGGGAGGGAGCAGCCACGGAGTTAGAGGC |
| | | CCTTGGGGTTTCTCTTTTCCACTGACAGGCTTTCCCAGGCAGCTGGCTAGTTCATT |
| | | CCCTCCCCAGCCAGGTGCAGGCGTAGGAATATGGACATCTGGTTGCTTTGGCCTG |
| | | CTGCCCTCTTTCAGGGGTCCTAAGCCCACAATCATGCCTCCCTAAGACCTTGGCA |
| | | TCCTTCCCTCTAAGCCGTTGGCACCTCTGTGCCACCTCTCACACTGGCTCCAGACA |
| | | CACAGCCTGTGCTTTTGGAGCTGAGATCACTCGCTTCACCCTCCTCATCTTTGTTC |
| | | TCCAAGTAAAGCCACGAGGTCGGGGCGAGGGCAGAGGTGATCACCTGCGTGTCC |
| | | CATCTACAGACCTGCAGCTTCATAAAACTTCTGATTTCTCTTCAGCTTTGAAAAG |
| | | GGTTACCCTGGGCACTGGCCTAGAGCCTCACCTCCTAATAGACTTAGCCCCATGA |
| | | GTTTGCCATGTTGAGCAGGACTATTTCTGGCACTTGCAAGTCCCATGATTTCTTCG |
| | | GTAATTCTGAGGGTGGGGGGAGGGACATGAAATCATCTTAGCTTAGCTTTCTGTC |
| | | TGTGAATGTCTATATAGTGTATTGTGTGTTTTAACAAATGATTTACACTGACTGTT |
| | | GCTGTAAAAGTGAATTTGGAAATAAAGTTATTACTCTGATTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 403 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGAATCTCCCCTGCAGACCCCCACTGAGGACGGATCTGAGGAACC |
| | | GGGCTCTGAAACCTCTGATGCTAAGAGCACTCCAACAGCGGAAGCTGAAGAAGC |
| | | AGGCATTGGAGACACCCCCAGCCTGGAAGACGAAGCTGCTGGTCACGTGACCCA |
| | | AGCTCGCATGGTCAGTAAAAGCAAAGACGGGACTGGAAGCGATGACAAAAAAG |
| | | CCAAGGGGGCTGATGGTAAAACGAAGATCGCCACACCGCGGGGAGCAGCCCCTC |
| | | CAGGCCAGAAGGGCCAGGCCAACGCCACCAGGATTCCAGCAAAAACCCCGCCCG |
| | | CTCCAAAGACACCACCCAGCTCTGGTGAACCTCCAAAATCAGGGGATCGCAGCG |
| | | GCTACAGCAGCCCCGGCTCCCCAGGCACTCCCGGCAGCCGCTCCCGCACCCCGTC |
| | | CCTTCCAACCCCACCCACCCGGGAGCCCAAGAAGGTGGCAGTGGTCCGTACTCC |
| | | ACCCAAGTCGCCGTCTTCCGCCAAGAGCCGCCTGCAGACAGCCCCCGTGCCCATG |
| | | CCAGACCTGAAGAATGTCAAGTCCAAGATCGGCTCCACTGAGAACCTGAAGCAC |
| | | CAGCCGGGAGGCGGGAAGGTGCAAATAGTCTACAAACCAGTTGACCTGAGCAAG |
| | | GTGACCTCCAAGTGTGGCTCATTAGGCAACATCCATCATAAACCAGGAGGTGGC |
| | | CAGGTGGAAGTAAAATCTGAGAAGCTTGACTTCAAGGACAGAGTCCAGTCGAAG |
| | | ATTGGGTCCCTGGACAATATCACCCACGTCCCTGGCGGAGGAAATAAAAAGATT |
| | | GAAACCCACAAGCTGACCTTCCGCGAGAACGCCAAAGCCAAGACAGACCACGG |
| | | GGCGGAGATCGTGTACAAGTCGCCAGTGGTGTCTGGGGACACGTCTCCACGGCA |
| | | TCTCAGCAATGTCTCCTCCACCGGCAGCATCGACATGGTAGACTCGCCCCAGCTC |
| | | GCCACGCTAGCTGACGAGGTGTCTGCCTCCCTGGCCAAGCAGGGTTTGTGATCAG |
| | | GCCCCTGGGGCGGTCAATAATTGTGGAGAGGAGAGAATGAGAGAGTGTGGAAA |
| | | AAAAAAGAATAATGACCCGGCCCCCGCCCTCTGCCCCCAGCTGCTCCTCGCAGTT |
| | | CGGTTAATTGGTTAATCACTTAACCTGCTTTTGTCACTCGGCTTTGGCTCGGGACT |
| | | TCAAAATCAGTGATGGGAGTAAGAGCAAATTTCATCTTTCCAAATTGATGGGTGG |
| | | GCTAGTAATAAAATATTTAAAAAAAAACATTCAAAAACATGGCCACATCCAACA |
| | | TTTCCTCAGGCAATTCCTTTTGATTCTTTTTTCTTCCCCCTCCATGTAGAAGAGGG |
| | | AGAAGGAGAGGCTCTGAAAGCTGCTTCTGGGGGATTTCAAGGGACTGGGGGTGC |
| | | CAACCACCTCTGGCCCTGTTGTGGGGGTGTCACAGAGGCAGTGGCAGCAACAAA |
| | | GGATTTGAAACTTGGTGTGTTCGTGGAGCCACAGGCAGACGATGTCAACCTTGTG |
| | | TGAGTGTGACGGGGGTTGGGGTGGGGGGGGAGGCCACGGGGGAGGCCGAGGCA |
| | | GGGGCTGGGCAGAGGGGAGAGGAAGCACAAGAAGTGGGAGTGGGAGAGGAAG |
| | | CCACGTGCTGGAGAGTAGACATCCCCCTCCTTGCCGCTGGGAGAGCCAAGGCCT |
| | | ATGCCACCTGCAGCGTCTGAGCGGCCGCCTGTCCTTGGTGGCCGGGGGTGGGGG |
| | | CCTGCTGTGGGTCAGTGTGCCACCCTCTGCAGGGCAGCCTGTGGGAGAAGGGAC |
| | | AGCGGGTAAAAAGAGAAGGCAAGCTGGCAGGAGGGTGGCACTTCGTGGATGAC |
| | | CTCCTTAGAAAAGACTGACCTTGATGTCTTGAGAGCGCTGGCCTCTTCCTCCCTC |
| | | CCTGCAGGGTAGGGGGCCTGAGTTGAGGGGCTTCCCTCTGCTCCACAGAAACCCT |
| | | GTTTTATTGAGTTCTGAAGGTTGGAACTGCTGCCATGATTTTGGCCACTTTGCAG |
| | | ACCTGGGACTTTAGGGCTAACCAGTTCTCTTTGTAAGGACTTGTGCCTCTTGGGA |
| | | GACGTCCACCCGTTTCCAAGCCTGGGCCACTGGCATCTCTGGAGTGTGTGGGGGT |
| | | CTGGGAGGCAGGTCCCGAGCCCCCTGTCCTTCCCACGGCCACTGCAGTCACCCCG |
| | | TCTGCGCCGCTGTGCTGTTGTCTGCCGTGAGAGCCCAATCACTGCCTATACCCCT |
| | | CATCACACGTCACAATGTCCCGAATTCCCAGCCTCACCACCCCTTCTCAGTAATG |
| | | ACCCTGGTTGGTTGCAGGAGGTACCTACTCCATACTGAGGGTGAAATTAAGGGA |
| | | AGGCAAAGTCCAGGCACAAGAGTGGGACCCCAGCCTCTCACTCTCAGTTCCACT |
| | | CATCCAACTGGGACCCTCACCACGAATCTCATGATCTGATTCGGTTCCCTGTCTC |
| | | CTCCTCCCGTCACAGATGTGAGCCAGGGCACTGCTCAGCTGTGACCCTAGGTGTT |
| | | TCTGCCTTGTTGACATGGAGAGAGCCCTTTCCCCTGAGAAGGCCTGGCCCCTTCC |
| | | TGTGCTGAGCCCACAGCAGCAGGCTGGGTGTCTTGGTTGTCAGTGGTGGCACCAG |
| | | GATGGAAGGGCAAGGCACCCAGGGCAGGCCCACAGTCCCGCTGTCCCCCACTTG |
| | | CACCCTAGCTTGTAGCTGCCAACCTCCCAGACAGCCCAGCCCGCTGCTCAGCTCC |
| | | ACATGCATAGTATCAGCCCTCCACACCCGACAAAGGGGAACACACCCCCTTGGA |
| | | AATGGTTCTTTTCCCCCAGTCCCAGCTGGAAGCCATGCTGTCTGTTCTGCTGGAG |
| | | CAGCTGAACATATACATAGATGTTGCCCTGCCCTCCCCATCTGCACCCTGTTGAG |
| | | TTGTAGTTGGATTTGTCTGTTTATGCTTGGATTCACCAGAGTGACTATGATAGTGA |
| | | AAAGAAAAAAAAAAAAAAAAAAGGACGCATGTATCTTGAAATGCTTGTAAAGA |
| | | GGTTTCTAACCCACCCTCACGAGGTGTCTCTCACCCCCACACTGGGACTCGTGTG |
| | | GCCTGTGTGGTGCCACCCTGCTGGGGCCTCCCAAGTTTTGAAAGGCTTTCCTCAG |
| | | CACCTGGGACCCAACAGAGACCAGCTTCTAGCAGCTAAGGAGGCCGTTCAGCTG |
| | | TGACGAAGGCCTGAAGCACAGGATTAGGACTGAAGCGATGATGTCCCCTTCCCT |
| | | ACTTCCCCTTGGGGCTCCCTGTGTCAGGGCACAGACTAGGTCTTGTGGCTGGTCT |
| | | GGCTTGCGGCGCGAGGATGGTTCTCTCTGGTCATAGCCCGAAGTCTCATGGCAGT |
| | | CCCAAAGGAGGCTTACAACTCCTGCATCACAAGAAAAAGGAAGCCACTGCCAGC |
| | | TGGGGGGATCTGCAGCTCCCAGAAGCTCCGTGAGCCTCAGCCACCCCTCAGACT |
| | | GGGTTCCTCTCCAAGCTCGCCCTCTGGAGGGGCAGCGCAGCCTCCCACCAAGGG |
| | | CCCTGCGACCACAGCAGGGATTGGGATGAATTGCCTGTCCTGGATCTGCTCTAGA |
| | | GGCCCAAGCTGCCTGCCTGAGGAAGGATGACTTGACAAGTCAGGAGACACTGTT |
| | | CCCAAAGCCTTGACCAGAGCACCTCAGCCCGCTGACCTTGCACAAACTCCATCTG |
| | | CTGCCATGAGAAAAGGGAAGCCGCCTTTGCAAAACATTGCTGCCTAAAGAAACT |
| | | CAGCAGCCTCAGGCCCAATTCTGCCACTTCTGGTTTGGGTACAGTTAAAGGCAAC |
| | | CCTGAGGGACTTGGCAGTAGAAATCCAGGGCCTCCCCTGGGGCTGGCAGCTTCG |
| | | TGTGCAGCTAGAGCTTTACCTGAAAGGAAGTCTCTGGGCCCAGAACTCTCCACCA |
| | | AGAGCCTCCCTGCCGTTCGCTGAGTCCCAGCAATTCTCCTAAGTTGAAGGGATCT |
| | | GAGAAGGAGAAGGAAATGTGGGGTAGATTTGGTGGTGGTTAGAGATATGCCCCC |
| | | CTCATTACTGCCAACAGTTTCGGCTGCATTTCTTCACGCACCTCGGTTCCTCTTCC |
| | | TGAAGTTCTTGTGCCCTGCTCTTCAGCACCATGGGCCTTCTTATACGGAAGGCTCT |
| | | GGGATCTCCCCCTTGTGGGGCAGGCTCTTGGGGCCAGCCTAAGATCATGGTTTAG |
| | | GGTGATCAGTGCTGGCAGATAAATTGAAAAGGCACGCTGGCTTGTGATCTTAAA |
| | | TGAGGACAATCCCCCCAGGGCTGGGCACTCCTCCCCTCCCCTCACTTCTCCCACC |
| | | TGCAGAGCCAGTGTCCTTGGGTGGGCTAGATAGGATATACTGTATGCCGGCTCCT |
| | | TCAAGCTGCTGACTCACTTTATCAATAGTTCCATTTAAATTGACTTCAGTGGTGA |
| | | GACTGTATCCTGTTTGCTATTGCTTGTTGTGCTATGGGGGGAGGGGGGAGGAATG |
| | | TGTAAGATAGTTAACATGGGCAAAGGGAGATCTTGGGGTGCAGCACTTAAACTG |
| | | CCTCGTAACCCTTTTCATGATTTCAACCACATTTGCTAGAGGGAGGGAGCAGCCA |
| | | CGGAGTTAGAGGCCCTTGGGGTTTCTCTTTTCCACTGACAGGCTTTCCCAGGCAG |
| | | CTGGCTAGTTCATTCCCTCCCCAGCCAGGTGCAGGCGTAGGAATATGGACATCTG |
| | | GTTGCTTTGGCCTGCTGCCCTCTTTCAGGGGTCCTAAGCCCACAATCATGCCTCCC |
| | | TAAGACCTTGGCATCCTTCCCTCTAAGCCGTTGGCACCTCTGTGCCACCTCTCACA |
| | | CTGGCTCCAGACACACAGCCTGTGCTTTTGGAGCTGAGATCACTCGCTTCACCCT |
| | | CCTCATCTTTGTTCTCCAAGTAAAGCCACGAGGTCGGGGCGAGGGCAGAGGTGA |
| | | TCACCTGCGTGTCCCATCTACAGACCTGCAGCTTCATAAAACTTCTGATTTCTCTT |
| | | CAGCTTTGAAAAGGGTTACCCTGGGCACTGGCCTAGAGCCTCACCTCCTAATAGA |
| | | CTTAGCCCCATGAGTTTGCCATGTTGAGCAGGACTATTTCTGGCACTTGCAAGTC |
| | | CCATGATTTCTTCGGTAATTCTGAGGGTGGGGGGAGGGACATGAAATCATCTTAG |
| | | CTTAGCTTTCTGTCTGTGAATGTCTATATAGTGTATTGTGTGTTTTAACAAATGAT |
| | | TTACACTGACTGTTGCTGTAAAAGTGAATTTGGAAATAAAGTTATTACTCTGATT |
| | | AAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 404 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGAATCTCCCCTGCAGACCCCCACTGAGGACGGATCTGAGGAACC |
| | | GGGCTCTGAAACCTCTGATGCTAAGAGCACTCCAACAGCGGAAGATGTGACAGC |
| | | ACCCTTAGTGGATGAGGGAGCTCCCGGCAAGCAGGCTGCCGCGCAGCCCCACAC |
| | | GGAGATCCCAGAAGGAACCACAGCTGAAGAAGCAGGCATTGGAGACACCCCCA |
| | | GCCTGGAAGACGAAGCTGCTGGTCACGTGACCCAAGCTCGCATGGTCAGTAAAA |
| | | GCAAAGACGGGACTGGAAGCGATGACAAAAAAGCCAAGGGGGCTGATGGTAAA |
| | | ACGAAGATCGCCACACCGCGGGGAGCAGCCCCTCCAGGCCAGAAGGGCCAGGC |
| | | CAACGCCACCAGGATTCCAGCAAAAACCCCGCCCGCTCCAAAGACACCACCCAG |
| | | CTCTGGTGAACCTCCAAAATCAGGGGATCGCAGCGGCTACAGCAGCCCCGGCTC |
| | | CCCAGGCACTCCCGGCAGCCGCTCCCGCACCCCGTCCCTTCCAACCCCACCCACC |
| | | CGGGAGCCCAAGAAGGTGGCAGTGGTCCGTACTCCACCCAAGTCGCCGTCTTCC |
| | | GCCAAGAGCCGCCTGCAGACAGCCCCCGTGCCCATGCCAGACCTGAAGAATGTC |
| | | AAGTCCAAGATCGGCTCCACTGAGAACCTGAAGCACCAGCCGGGAGGCGGGAA |
| | | GGTGCAAATAGTCTACAAACCAGTTGACCTGAGCAAGGTGACCTCCAAGTGTGG |
| | | CTCATTAGGCAACATCCATCATAAACCAGGAGGTGGCCAGGTGGAAGTAAAATC |
| | | TGAGAAGCTTGACTTCAAGGACAGAGTCCAGTCGAAGATTGGGTCCCTGGACAA |
| | | TATCACCCACGTCCCTGGCGGAGGAAATAAAAAGATTGAAACCCACAAGCTGAC |
| | | CTTCCGCGAGAACGCCAAAGCCAAGACAGACCACGGGGCGGAGATCGTGTACAA |
| | | GTCGCCAGTGGTGTCTGGGGACACGTCTCCACGGCATCTCAGCAATGTCTCCTCC |
| | | ACCGGCAGCATCGACATGGTAGACTCGCCCCAGCTCGCCACGCTAGCTGACGAG |
| | | GTGTCTGCCTCCCTGGCCAAGCAGGGTTTGTGATCAGGCCCCTGGGGCGGTCAAT |
| | | AATTGTGGAGAGGAGAGAATGAGAGAGTGTGGAAAAAAAAAGAATAATGACCC |
| | | GGCCCCCGCCCTCTGCCCCCAGCTGCTCCTCGCAGTTCGGTTAATTGGTTAATCA |
| | | CTTAACCTGCTTTTGTCACTCGGCTTTGGCTCGGGACTTCAAAATCAGTGATGGG |
| | | AGTAAGAGCAAATTTCATCTTTCCAAATTGATGGGTGGGCTAGTAATAAAATATT |
| | | TAAAAAAAAACATTCAAAAACATGGCCACATCCAACATTTCCTCAGGCAATTCCT |
| | | TTTGATTCTTTTTTCTTCCCCCTCCATGTAGAAGAGGGAGAAGGAGAGGCTCTGA |
| | | AAGCTGCTTCTGGGGGATTTCAAGGGACTGGGGGTGCCAACCACCTCTGGCCCTG |
| | | TTGTGGGGGTGTCACAGAGGCAGTGGCAGCAACAAAGGATTTGAAACTTGGTGT |
| | | GTTCGTGGAGCCACAGGCAGACGATGTCAACCTTGTGTGAGTGTGACGGGGGTT |
| | | GGGGTGGGGGGGGAGGCCACGGGGGAGGCCGAGGCAGGGGCTGGGCAGAGGGG |
| | | AGAGGAAGCACAAGAAGTGGGAGTGGGAGAGGAAGCCACGTGCTGGAGAGTAG |
| | | ACATCCCCCTCCTTGCCGCTGGGAGAGCCAAGGCCTATGCCACCTGCAGCGTCTG |
| | | AGCGGCCGCCTGTCCTTGGTGGCCGGGGGTGGGGGCCTGCTGTGGGTCAGTGTG |
| | | CCACCCTCTGCAGGGCAGCCTGTGGGAGAAGGGACAGCGGGTAAAAAGAGAAG |
| | | GCAAGCTGGCAGGAGGGTGGCACTTCGTGGATGACCTCCTTAGAAAAGACTGAC |
| | | CTTGATGTCTTGAGAGCGCTGGCCTCTTCCTCCCTCCCTGCAGGGTAGGGGGCCT |
| | | GAGTTGAGGGGCTTCCCTCTGCTCCACAGAAACCCTGTTTTATTGAGTTCTGAAG |
| | | GTTGGAACTGCTGCCATGATTTTGGCCACTTTGCAGACCTGGGACTTTAGGGCTA |
| | | ACCAGTTCTCTTTGTAAGGACTTGTGCCTCTTGGGAGACGTCCACCCGTTTCCAA |
| | | GCCTGGGCCACTGGCATCTCTGGAGTGTGTGGGGGTCTGGGAGGCAGGTCCCGA |
| | | GCCCCCTGTCCTTCCCACGGCCACTGCAGTCACCCCGTCTGCGCCGCTGTGCTGTT |
| | | GTCTGCCGTGAGAGCCCAATCACTGCCTATACCCCTCATCACACGTCACAATGTC |
| | | CCGAATTCCCAGCCTCACCACCCCTTCTCAGTAATGACCCTGGTTGGTTGCAGGA |
| | | GGTACCTACTCCATACTGAGGGTGAAATTAAGGGAAGGCAAAGTCCAGGCACAA |
| | | GAGTGGGACCCCAGCCTCTCACTCTCAGTTCCACTCATCCAACTGGGACCCTCAC |
| | | CACGAATCTCATGATCTGATTCGGTTCCCTGTCTCCTCCTCCCGTCACAGATGTGA |
| | | GCCAGGGCACTGCTCAGCTGTGACCCTAGGTGTTTCTGCCTTGTTGACATGGAGA |
| | | GAGCCCTTTCCCCTGAGAAGGCCTGGCCCCTTCCTGTGCTGAGCCCACAGCAGCA |
| | | GGCTGGGTGTCTTGGTTGTCAGTGGTGGCACCAGGATGGAAGGGCAAGGCACCC |
| | | AGGGCAGGCCCACAGTCCCGCTGTCCCCCACTTGCACCCTAGCTTGTAGCTGCCA |
| | | ACCTCCCAGACAGCCCAGCCCGCTGCTCAGCTCCACATGCATAGTATCAGCCCTC |
| | | CACACCCGACAAAGGGGAACACACCCCCTTGGAAATGGTTCTTTTCCCCCAGTCC |
| | | CAGCTGGAAGCCATGCTGTCTGTTCTGCTGGAGCAGCTGAACATATACATAGATG |
| | | TTGCCCTGCCCTCCCCATCTGCACCCTGTTGAGTTGTAGTTGGATTTGTCTGTTTA |
| | | TGCTTGGATTCACCAGAGTGACTATGATAGTGAAAAGAAAAAAAAAAAAAAAA |
| | | AAGGACGCATGTATCTTGAAATGCTTGTAAAGAGGTTTCTAACCCACCCTCACGA |
| | | GGTGTCTCTCACCCCCACACTGGGACTCGTGTGGCCTGTGTGGTGCCACCCTGCT |
| | | GGGGCCTCCCAAGTTTTGAAAGGCTTTCCTCAGCACCTGGGACCCAACAGAGAC |
| | | CAGCTTCTAGCAGCTAAGGAGGCCGTTCAGCTGTGACGAAGGCCTGAAGCACAG |
| | | GATTAGGACTGAAGCGATGATGTCCCCTTCCCTACTTCCCCTTGGGGCTCCCTGT |
| | | GTCAGGGCACAGACTAGGTCTTGTGGCTGGTCTGGCTTGCGGCGCGAGGATGGTT |
| | | CTCTCTGGTCATAGCCCGAAGTCTCATGGCAGTCCCAAAGGAGGCTTACAACTCC |
| | | TGCATCACAAGAAAAAGGAAGCCACTGCCAGCTGGGGGGATCTGCAGCTCCCAG |
| | | AAGCTCCGTGAGCCTCAGCCACCCCTCAGACTGGGTTCCTCTCCAAGCTCGCCCT |
| | | CTGGAGGGGCAGCGCAGCCTCCCACCAAGGGCCCTGCGACCACAGCAGGGATTG |
| | | GGATGAATTGCCTGTCCTGGATCTGCTCTAGAGGCCCAAGCTGCCTGCCTGAGGA |
| | | AGGATGACTTGACAAGTCAGGAGACACTGTTCCCAAAGCCTTGACCAGAGCACC |
| | | TCAGCCCGCTGACCTTGCACAAACTCCATCTGCTGCCATGAGAAAAGGGAAGCC |
| | | GCCTTTGCAAAACATTGCTGCCTAAAGAAACTCAGCAGCCTCAGGCCCAATTCTG |
| | | CCACTTCTGGTTTGGGTACAGTTAAAGGCAACCCTGAGGGACTTGGCAGTAGAA |
| | | ATCCAGGGCCTCCCCTGGGGCTGGCAGCTTCGTGTGCAGCTAGAGCTTTACCTGA |
| | | AAGGAAGTCTCTGGGCCCAGAACTCTCCACCAAGAGCCTCCCTGCCGTTCGCTGA |
| | | GTCCCAGCAATTCTCCTAAGTTGAAGGGATCTGAGAAGGAGAAGGAAATGTGGG |
| | | GTAGATTTGGTGGTGGTTAGAGATATGCCCCCCTCATTACTGCCAACAGTTTCGG |
| | | CTGCATTTCTTCACGCACCTCGGTTCCTCTTCCTGAAGTTCTTGTGCCCTGCTCTTC |
| | | AGCACCATGGGCCTTCTTATACGGAAGGCTCTGGGATCTCCCCCTTGTGGGGCAG |
| | | GCTCTTGGGGCCAGCCTAAGATCATGGTTTAGGGTGATCAGTGCTGGCAGATAA |
| | | ATTGAAAAGGCACGCTGGCTTGTGATCTTAAATGAGGACAATCCCCCCAGGGCT |
| | | GGGCACTCCTCCCCTCCCCTCACTTCTCCCACCTGCAGAGCCAGTGTCCTTGGGT |
| | | GGGCTAGATAGGATATACTGTATGCCGGCTCCTTCAAGCTGCTGACTCACTTTAT |
| | | CAATAGTTCCATTTAAATTGACTTCAGTGGTGAGACTGTATCCTGTTTGCTATTGC |
| | | TTGTTGTGCTATGGGGGGAGGGGGGAGGAATGTGTAAGATAGTTAACATGGGCA |
| | | AAGGGAGATCTTGGGGTGCAGCACTTAAACTGCCTCGTAACCCTTTTCATGATTT |
| | | CAACCACATTTGCTAGAGGGAGGGAGCAGCCACGGAGTTAGAGGCCCTTGGGGT |
| | | TTCTCTTTTCCACTGACAGGCTTTCCCAGGCAGCTGGCTAGTTCATTCCCTCCCCA |
| | | GCCAGGTGCAGGCGTAGGAATATGGACATCTGGTTGCTTTGGCCTGCTGCCCTCT |
| | | TTCAGGGGTCCTAAGCCCACAATCATGCCTCCCTAAGACCTTGGCATCCTTCCCT |
| | | CTAAGCCGTTGGCACCTCTGTGCCACCTCTCACACTGGCTCCAGACACACAGCCT |
| | | GTGCTTTTGGAGCTGAGATCACTCGCTTCACCCTCCTCATCTTTGTTCTCCAAGTA |
| | | AAGCCACGAGGTCGGGGCGAGGGCAGAGGTGATCACCTGCGTGTCCCATCTACA |
| | | GACCTGCAGCTTCATAAAACTTCTGATTTCTCTTCAGCTTTGAAAAGGGTTACCCT |
| | | GGGCACTGGCCTAGAGCCTCACCTCCTAATAGACTTAGCCCCATGAGTTTGCCAT |
| | | GTTGAGCAGGACTATTTCTGGCACTTGCAAGTCCCATGATTTCTTCGGTAATTCTG |
| | | AGGGTGGGGGGAGGGACATGAAATCATCTTAGCTTAGCTTTCTGTCTGTGAATGT |
| | | CTATATAGTGTATTGTGTGTTTTAACAAATGATTTACACTGACTGTTGCTGTAAAA |
| | | GTGAATTTGGAAATAAAGTTATTACTCTGATTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 405 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGAATCTCCCCTGCAGACCCCCACTGAGGACGGATCTGAGGAACC |
| | | GGGCTCTGAAACCTCTGATGCTAAGAGCACTCCAACAGCGGAAGCTGAAGAAGC |
| | | AGGCATTGGAGACACCCCCAGCCTGGAAGACGAAGCTGCTGGTCACGTGACCCA |
| | | AGAGGAGTTGAGAGTTCCGGGCCGGCAGAGGAAGGCGCCTGAAAGGCCCCTGG |
| | | CCAATGAGATTAGCGCCCACGTCCAGCCTGGACCCTGCGGAGAGGCCTCTGGGG |
| | | TCTCTGGGCCGTGCCTCGGGGAGAAAGAGCCAGAAGCTCCCGTCCCGCTGACCG |
| | | CGAGCCTTCCTCAGCACCGTCCCGTTTGCCCAGCGCCTCCTCCAACAGGAGGCCC |
| | | TCAGGAGCCCTCCCTGGAGTGGGGACAAAAAGGCGGGGACTGGGCCGAGAAGG |
| | | GTCCGGCCTTTCCGAAGCCCGCCACCACTGCGTATCTCCACACAGAGCCTGAAAG |
| | | TGGTAAGGTGGTCCAGGAAGGCTTCCTCCGAGAGCCAGGCCCCCCAGGTCTGAG |
| | | CCACCAGCTCATGTCCGGCATGCCTGGGGCTCCCCTCCTGCCTGAGGGCCCCAGA |
| | | GAGGCCACACGCCAACCTTCGGGGACAGGACCTGAGGACACAGAGGGCGGCCG |
| | | CCACGCCCCTGAGCTGCTCAAGCACCAGCTTCTAGGAGACCTGCACCAGGAGGG |
| | | GCCGCCGCTGAAGGGGGCAGGGGGCAAAGAGAGGCCGGGGAGCAAGGAGGAG |
| | | GTGGATGAAGACCGCGACGTCGATGAGTCCTCCCCCCAAGACTCCCCTCCCTCCA |
| | | AGGCCTCCCCAGCCCAAGATGGGCGGCCTCCCCAGACAGCCGCCAGAGAAGCCA |
| | | CCAGCATCCCAGGCTTCCCAGCGGAGGGTGCCATCCCCCTCCCTGTGGATTTCCT |
| | | CTCCAAAGTTTCCACAGAGATCCCAGCCTCAGAGCCCGACGGGCCCAGTGTAGG |
| | | GCGGGCCAAAGGGCAGGATGCCCCCCTGGAGTTCACGTTTCACGTGGAAATCAC |
| | | ACCCAACGTGCAGAAGGAGCAGGCGCACTCGGAGGAGCATTTGGGAAGGGCTG |
| | | CATTTCCAGGGGCCCCTGGAGAGGGGCCAGAGGCCCGGGGCCCCTCTTTGGGAG |
| | | AGGACACAAAAGAGGCTGACCTTCCAGAGCCCTCTGAAAAGCAGCCTGCTGCTG |
| | | CTCCGCGGGGGAAGCCCGTCAGCCGGGTCCCTCAACTCAAAGCTCGCATGGTCA |
| | | GTAAAAGCAAAGACGGGACTGGAAGCGATGACAAAAAAGCCAAGACATCCACA |
| | | CGTTCCTCTGCTAAAACCTTGAAAAATAGGCCTTGCCTTAGCCCCAAACACCCCA |
| | | CTCCTGGTAGCTCAGACCCTCTGATCCAACCCTCCAGCCCTGCTGTGTGCCCAGA |
| | | GCCACCTTCCTCTCCTAAATACGTCTCTTCTGTCACTTCCCGAACTGGCAGTTCTG |
| | | GAGCAAAGGAGATGAAACTCAAGGGGGCTGATGGTAAAACGAAGATCGCCACA |
| | | CCGCGGGGAGCAGCCCCTCCAGGCCAGAAGGGCCAGGCCAACGCCACCAGGATT |
| | | CCAGCAAAAACCCCGCCCGCTCCAAAGACACCACCCAGCTCTGGTGAACCTCCA |
| | | AAATCAGGGGATCGCAGCGGCTACAGCAGCCCCGGCTCCCCAGGCACTCCCGGC |
| | | AGCCGCTCCCGCACCCCGTCCCTTCCAACCCCACCCACCCGGGAGCCCAAGAAG |
| | | GTGGCAGTGGTCCGTACTCCACCCAAGTCGCCGTCTTCCGCCAAGAGCCGCCTGC |
| | | AGACAGCCCCCGTGCCCATGCCAGACCTGAAGAATGTCAAGTCCAAGATCGGCT |
| | | CCACTGAGAACCTGAAGCACCAGCCGGGAGGCGGGAAGGTGCAGATAATTAATA |
| | | AGAAGCTGGATCTTAGCAACGTCCAGTCCAAGTGTGGCTCAAAGGATAATATCA |
| | | AACACGTCCCGGGAGGCGGCAGTGTGCAAATAGTCTACAAACCAGTTGACCTGA |
| | | GCAAGGTGACCTCCAAGTGTGGCTCATTAGGCAACATCCATCATAAACCAGGAG |
| | | GTGGCCAGGTGGAAGTAAAATCTGAGAAGCTTGACTTCAAGGACAGAGTCCAGT |
| | | CGAAGATTGGGTCCCTGGACAATATCACCCACGTCCCTGGCGGAGGAAATAAAA |
| | | AGATTGAAACCCACAAGCTGACCTTCCGCGAGAACGCCAAAGCCAAGACAGACC |
| | | ACGGGGCGGAGATCGTGTACAAGTCGCCAGTGGTGTCTGGGGACACGTCTCCAC |
| | | GGCATCTCAGCAATGTCTCCTCCACCGGCAGCATCGACATGGTAGACTCGCCCCA |
| | | GCTCGCCACGCTAGCTGACGAGGTGTCTGCCTCCCTGGCCAAGCAGGGTTTGTGA |
| | | TCAGGCCCCTGGGGCGGTCAATAATTGTGGAGAGGAGAGAATGAGAGAGTGTGG |
| | | AAAAAAAAAGAATAATGACCCGGCCCCCGCCCTCTGCCCCCAGCTGCTCCTCGC |
| | | AGTTCGGTTAATTGGTTAATCACTTAACCTGCTTTTGTCACTCGGCTTTGGCTCGG |
| | | GACTTCAAAATCAGTGATGGGAGTAAGAGCAAATTTCATCTTTCCAAATTGATGG |
| | | GTGGGCTAGTAATAAAATATTTAAAAAAAAACATTCAAAAACATGGCCACATCC |
| | | AACATTTCCTCAGGCAATTCCTTTTGATTCTTTTTTCTTCCCCCTCCATGTAGAAG |
| | | AGGGAGAAGGAGAGGCTCTGAAAGCTGCTTCTGGGGGATTTCAAGGGACTGGGG |
| | | GTGCCAACCACCTCTGGCCCTGTTGTGGGGGTGTCACAGAGGCAGTGGCAGCAA |
| | | CAAAGGATTTGAAACTTGGTGTGTTCGTGGAGCCACAGGCAGACGATGTCAACC |
| | | TTGTGTGAGTGTGACGGGGGTTGGGGTGGGGGGGGAGGCCACGGGGGAGGCCG |
| | | AGGCAGGGGCTGGGCAGAGGGGAGAGGAAGCACAAGAAGTGGGAGTGGGAGA |
| | | GGAAGCCACGTGCTGGAGAGTAGACATCCCCCTCCTTGCCGCTGGGAGAGCCAA |
| | | GGCCTATGCCACCTGCAGCGTCTGAGCGGCCGCCTGTCCTTGGTGGCCGGGGGTG |
| | | GGGGCCTGCTGTGGGTCAGTGTGCCACCCTCTGCAGGGCAGCCTGTGGGAGAAG |
| | | GGACAGCGGGTAAAAAGAGAAGGCAAGCTGGCAGGAGGGTGGCACTTCGTGGA |
| | | TGACCTCCTTAGAAAAGACTGACCTTGATGTCTTGAGAGCGCTGGCCTCTTCCTC |
| | | CCTCCCTGCAGGGTAGGGGGCCTGAGTTGAGGGGCTTCCCTCTGCTCCACAGAAA |
| | | CCCTGTTTTATTGAGTTCTGAAGGTTGGAACTGCTGCCATGATTTTGGCCACTTTG |
| | | CAGACCTGGGACTTTAGGGCTAACCAGTTCTCTTTGTAAGGACTTGTGCCTCTTG |
| | | GGAGACGTCCACCCGTTTCCAAGCCTGGGCCACTGGCATCTCTGGAGTGTGTGGG |
| | | GGTCTGGGAGGCAGGTCCCGAGCCCCCTGTCCTTCCCACGGCCACTGCAGTCACC |
| | | CCGTCTGCGCCGCTGTGCTGTTGTCTGCCGTGAGAGCCCAATCACTGCCTATACC |
| | | CCTCATCACACGTCACAATGTCCCGAATTCCCAGCCTCACCACCCCTTCTCAGTA |
| | | ATGACCCTGGTTGGTTGCAGGAGGTACCTACTCCATACTGAGGGTGAAATTAAG |
| | | GGAAGGCAAAGTCCAGGCACAAGAGTGGGACCCCAGCCTCTCACTCTCAGTTCC |
| | | ACTCATCCAACTGGGACCCTCACCACGAATCTCATGATCTGATTCGGTTCCCTGT |
| | | CTCCTCCTCCCGTCACAGATGTGAGCCAGGGCACTGCTCAGCTGTGACCCTAGGT |
| | | GTTTCTGCCTTGTTGACATGGAGAGAGCCCTTTCCCCTGAGAAGGCCTGGCCCCT |
| | | TCCTGTGCTGAGCCCACAGCAGCAGGCTGGGTGTCTTGGTTGTCAGTGGTGGCAC |
| | | CAGGATGGAAGGGCAAGGCACCCAGGGCAGGCCCACAGTCCCGCTGTCCCCCAC |
| | | TTGCACCCTAGCTTGTAGCTGCCAACCTCCCAGACAGCCCAGCCCGCTGCTCAGC |
| | | TCCACATGCATAGTATCAGCCCTCCACACCCGACAAAGGGGAACACACCCCCTT |
| | | GGAAATGGTTCTTTTCCCCCAGTCCCAGCTGGAAGCCATGCTGTCTGTTCTGCTG |
| | | GAGCAGCTGAACATATACATAGATGTTGCCCTGCCCTCCCCATCTGCACCCTGTT |
| | | GAGTTGTAGTTGGATTTGTCTGTTTATGCTTGGATTCACCAGAGTGACTATGATA |
| | | GTGAAAAGAAAAAAAAAAAAAAAAAAGGACGCATGTATCTTGAAATGCTTGTA |
| | | AAGAGGTTTCTAACCCACCCTCACGAGGTGTCTCTCACCCCCACACTGGGACTCG |
| | | TGTGGCCTGTGTGGTGCCACCCTGCTGGGGCCTCCCAAGTTTTGAAAGGCTTTCC |
| | | TCAGCACCTGGGACCCAACAGAGACCAGCTTCTAGCAGCTAAGGAGGCCGTTCA |
| | | GCTGTGACGAAGGCCTGAAGCACAGGATTAGGACTGAAGCGATGATGTCCCCTT |
| | | CCCTACTTCCCCTTGGGGCTCCCTGTGTCAGGGCACAGACTAGGTCTTGTGGCTG |
| | | GTCTGGCTTGCGGCGCGAGGATGGTTCTCTCTGGTCATAGCCCGAAGTCTCATGG |
| | | CAGTCCCAAAGGAGGCTTACAACTCCTGCATCACAAGAAAAAGGAAGCCACTGC |
| | | CAGCTGGGGGGATCTGCAGCTCCCAGAAGCTCCGTGAGCCTCAGCCACCCCTCA |
| | | GACTGGGTTCCTCTCCAAGCTCGCCCTCTGGAGGGGCAGCGCAGCCTCCCACCAA |
| | | GGGCCCTGCGACCACAGCAGGGATTGGGATGAATTGCCTGTCCTGGATCTGCTCT |
| | | AGAGGCCCAAGCTGCCTGCCTGAGGAAGGATGACTTGACAAGTCAGGAGACACT |
| | | GTTCCCAAAGCCTTGACCAGAGCACCTCAGCCCGCTGACCTTGCACAAACTCCAT |
| | | CTGCTGCCATGAGAAAAGGGAAGCCGCCTTTGCAAAACATTGCTGCCTAAAGAA |
| | | ACTCAGCAGCCTCAGGCCCAATTCTGCCACTTCTGGTTTGGGTACAGTTAAAGGC |
| | | AACCCTGAGGGACTTGGCAGTAGAAATCCAGGGCCTCCCCTGGGGCTGGCAGCT |
| | | TCGTGTGCAGCTAGAGCTTTACCTGAAAGGAAGTCTCTGGGCCCAGAACTCTCCA |
| | | CCAAGAGCCTCCCTGCCGTTCGCTGAGTCCCAGCAATTCTCCTAAGTTGAAGGGA |
| | | TCTGAGAAGGAGAAGGAAATGTGGGGTAGATTTGGTGGTGGTTAGAGATATGCC |
| | | CCCCTCATTACTGCCAACAGTTTCGGCTGCATTTCTTCACGCACCTCGGTTCCTCT |
| | | TCCTGAAGTTCTTGTGCCCTGCTCTTCAGCACCATGGGCCTTCTTATACGGAAGG |
| | | CTCTGGGATCTCCCCCTTGTGGGGCAGGCTCTTGGGGCCAGCCTAAGATCATGGT |
| | | TTAGGGTGATCAGTGCTGGCAGATAAATTGAAAAGGCACGCTGGCTTGTGATCTT |
| | | AAATGAGGACAATCCCCCCAGGGCTGGGCACTCCTCCCCTCCCCTCACTTCTCCC |
| | | ACCTGCAGAGCCAGTGTCCTTGGGTGGGCTAGATAGGATATACTGTATGCCGGCT |
| | | CCTTCAAGCTGCTGACTCACTTTATCAATAGTTCCATTTAAATTGACTTCAGTGGT |
| | | GAGACTGTATCCTGTTTGCTATTGCTTGTTGTGCTATGGGGGGAGGGGGGAGGAA |
| | | TGTGTAAGATAGTTAACATGGGCAAAGGGAGATCTTGGGGTGCAGCACTTAAAC |
| | | TGCCTCGTAACCCTTTTCATGATTTCAACCACATTTGCTAGAGGGAGGGAGCAGC |
| | | CACGGAGTTAGAGGCCCTTGGGGTTTCTCTTTTCCACTGACAGGCTTTCCCAGGC |
| | | AGCTGGCTAGTTCATTCCCTCCCCAGCCAGGTGCAGGCGTAGGAATATGGACATC |
| | | TGGTTGCTTTGGCCTGCTGCCCTCTTTCAGGGGTCCTAAGCCCACAATCATGCCTC |
| | | CCTAAGACCTTGGCATCCTTCCCTCTAAGCCGTTGGCACCTCTGTGCCACCTCTCA |
| | | CACTGGCTCCAGACACACAGCCTGTGCTTTTGGAGCTGAGATCACTCGCTTCACC |
| | | CTCCTCATCTTTGTTCTCCAAGTAAAGCCACGAGGTCGGGGCGAGGGCAGAGGT |
| | | GATCACCTGCGTGTCCCATCTACAGACCTGCAGCTTCATAAAACTTCTGATTTCTC |
| | | TTCAGCTTTGAAAAGGGTTACCCTGGGCACTGGCCTAGAGCCTCACCTCCTAATA |
| | | GACTTAGCCCCATGAGTTTGCCATGTTGAGCAGGACTATTTCTGGCACTTGCAAG |
| | | TCCCATGATTTCTTCGGTAATTCTGAGGGTGGGGGGAGGGACATGAAATCATCTT |
| | | AGCTTAGCTTTCTGTCTGTGAATGTCTATATAGTGTATTGTGTGTTTTAACAAATG |
| | | ATTTACACTGACTGTTGCTGTAAAAGTGAATTTGGAAATAAAGTTATTACTCTGA |
| | | TTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 406 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGAATCTCCCCTGCAGACCCCCACTGAGGACGGATCTGAGGAACC |
| | | GGGCTCTGAAACCTCTGATGCTAAGAGCACTCCAACAGCGGAAGCTGAAGAAGC |
| | | AGGCATTGGAGACACCCCCAGCCTGGAAGACGAAGCTGCTGGTCACGTGACCCA |
| | | AGAGGAGTTGAGAGTTCCGGGCCGGCAGAGGAAGGCGCCTGAAAGGCCCCTGG |
| | | CCAATGAGATTAGCGCCCACGTCCAGCCTGGACCCTGCGGAGAGGCCTCTGGGG |
| | | TCTCTGGGCCGTGCCTCGGGGAGAAAGAGCCAGAAGCTCCCGTCCCGCTGACCG |
| | | CGAGCCTTCCTCAGCACCGTCCCGTTTGCCCAGCGCCTCCTCCAACAGGAGGCCC |
| | | TCAGGAGCCCTCCCTGGAGTGGGGACAAAAAGGCGGGGACTGGGCCGAGAAGG |
| | | GTCCGGCCTTTCCGAAGCCCGCCACCACTGCGTATCTCCACACAGAGCCTGAAAG |
| | | TGGTAAGGTGGTCCAGGAAGGCTTCCTCCGAGAGCCAGGCCCCCCAGGTCTGAG |
| | | CCACCAGCTCATGTCCGGCATGCCTGGGGCTCCCCTCCTGCCTGAGGGCCCCAGA |
| | | GAGGCCACACGCCAACCTTCGGGGACAGGACCTGAGGACACAGAGGGCGGCCG |
| | | CCACGCCCCTGAGCTGCTCAAGCACCAGCTTCTAGGAGACCTGCACCAGGAGGG |
| | | GCCGCCGCTGAAGGGGGCAGGGGGCAAAGAGAGGCCGGGGAGCAAGGAGGAG |
| | | GTGGATGAAGACCGCGACGTCGATGAGTCCTCCCCCCAAGACTCCCCTCCCTCCA |
| | | AGGCCTCCCCAGCCCAAGATGGGCGGCCTCCCCAGACAGCCGCCAGAGAAGCCA |
| | | CCAGCATCCCAGGCTTCCCAGCGGAGGGTGCCATCCCCCTCCCTGTGGATTTCCT |
| | | CTCCAAAGTTTCCACAGAGATCCCAGCCTCAGAGCCCGACGGGCCCAGTGTAGG |
| | | GCGGGCCAAAGGGCAGGATGCCCCCCTGGAGTTCACGTTTCACGTGGAAATCAC |
| | | ACCCAACGTGCAGAAGGAGCAGGCGCACTCGGAGGAGCATTTGGGAAGGGCTG |
| | | CATTTCCAGGGGCCCCTGGAGAGGGGCCAGAGGCCCGGGGCCCCTCTTTGGGAG |
| | | AGGACACAAAAGAGGCTGACCTTCCAGAGCCCTCTGAAAAGCAGCCTGCTGCTG |
| | | CTCCGCGGGGGAAGCCCGTCAGCCGGGTCCCTCAACTCAAAGCTCGCATGGTCA |
| | | GTAAAAGCAAAGACGGGACTGGAAGCGATGACAAAAAAGCCAAGGGGGCTGAT |
| | | GGTAAAACGAAGATCGCCACACCGCGGGGAGCAGCCCCTCCAGGCCAGAAGGG |
| | | CCAGGCCAACGCCACCAGGATTCCAGCAAAAACCCCGCCCGCTCCAAAGACACC |
| | | ACCCAGCTCTGGTGAACCTCCAAAATCAGGGGATCGCAGCGGCTACAGCAGCCC |
| | | CGGCTCCCCAGGCACTCCCGGCAGCCGCTCCCGCACCCCGTCCCTTCCAACCCCA |
| | | CCCACCCGGGAGCCCAAGAAGGTGGCAGTGGTCCGTACTCCACCCAAGTCGCCG |
| | | TCTTCCGCCAAGAGCCGCCTGCAGACAGCCCCCGTGCCCATGCCAGACCTGAAG |
| | | AATGTCAAGTCCAAGATCGGCTCCACTGAGAACCTGAAGCACCAGCCGGGAGGC |
| | | GGGAAGGTGCAAATAGTCTACAAACCAGTTGACCTGAGCAAGGTGACCTCCAAG |
| | | TGTGGCTCATTAGGCAACATCCATCATAAACCAGGAGGTGGCCAGGTGGAAGTA |
| | | AAATCTGAGAAGCTTGACTTCAAGGACAGAGTCCAGTCGAAGATTGGGTCCCTG |
| | | GACAATATCACCCACGTCCCTGGCGGAGGAAATAAAAAGATTGAAACCCACAAG |
| | | CTGACCTTCCGCGAGAACGCCAAAGCCAAGACAGACCACGGGGCGGAGATCGTG |
| | | TACAAGTCGCCAGTGGTGTCTGGGGACACGTCTCCACGGCATCTCAGCAATGTCT |
| | | CCTCCACCGGCAGCATCGACATGGTAGACTCGCCCCAGCTCGCCACGCTAGCTGA |
| | | CGAGGTGTCTGCCTCCCTGGCCAAGCAGGGTTTGTGATCAGGCCCCTGGGGCGGT |
| | | CAATAATTGTGGAGAGGAGAGAATGAGAGAGTGTGGAAAAAAAAAGAATAATG |
| | | ACCCGGCCCCCGCCCTCTGCCCCCAGCTGCTCCTCGCAGTTCGGTTAATTGGTTA |
| | | ATCACTTAACCTGCTTTTGTCACTCGGCTTTGGCTCGGGACTTCAAAATCAGTGAT |
| | | GGGAGTAAGAGCAAATTTCATCTTTCCAAATTGATGGGTGGGCTAGTAATAAAA |
| | | TATTTAAAAAAAAACATTCAAAAACATGGCCACATCCAACATTTCCTCAGGCAAT |
| | | TCCTTTTGATTCTTTTTTCTTCCCCCTCCATGTAGAAGAGGGAGAAGGAGAGGCT |
| | | CTGAAAGCTGCTTCTGGGGGATTTCAAGGGACTGGGGGTGCCAACCACCTCTGG |
| | | CCCTGTTGTGGGGGTGTCACAGAGGCAGTGGCAGCAACAAAGGATTTGAAACTT |
| | | GGTGTGTTCGTGGAGCCACAGGCAGACGATGTCAACCTTGTGTGAGTGTGACGG |
| | | GGGTTGGGGTGGGGGGGGAGGCCACGGGGGAGGCCGAGGCAGGGGCTGGGCAG |
| | | AGGGGAGAGGAAGCACAAGAAGTGGGAGTGGGAGAGGAAGCCACGTGCTGGAG |
| | | AGTAGACATCCCCCTCCTTGCCGCTGGGAGAGCCAAGGCCTATGCCACCTGCAGC |
| | | GTCTGAGCGGCCGCCTGTCCTTGGTGGCCGGGGGTGGGGGCCTGCTGTGGGTCA |
| | | GTGTGCCACCCTCTGCAGGGCAGCCTGTGGGAGAAGGGACAGCGGGTAAAAAGA |
| | | GAAGGCAAGCTGGCAGGAGGGTGGCACTTCGTGGATGACCTCCTTAGAAAAGAC |
| | | TGACCTTGATGTCTTGAGAGCGCTGGCCTCTTCCTCCCTCCCTGCAGGGTAGGGG |
| | | GCCTGAGTTGAGGGGCTTCCCTCTGCTCCACAGAAACCCTGTTTTATTGAGTTCT |
| | | GAAGGTTGGAACTGCTGCCATGATTTTGGCCACTTTGCAGACCTGGGACTTTAGG |
| | | GCTAACCAGTTCTCTTTGTAAGGACTTGTGCCTCTTGGGAGACGTCCACCCGTTTC |
| | | CAAGCCTGGGCCACTGGCATCTCTGGAGTGTGTGGGGGTCTGGGAGGCAGGTCC |
| | | CGAGCCCCCTGTCCTTCCCACGGCCACTGCAGTCACCCCGTCTGCGCCGCTGTGC |
| | | TGTTGTCTGCCGTGAGAGCCCAATCACTGCCTATACCCCTCATCACACGTCACAA |
| | | TGTCCCGAATTCCCAGCCTCACCACCCCTTCTCAGTAATGACCCTGGTTGGTTGC |
| | | AGGAGGTACCTACTCCATACTGAGGGTGAAATTAAGGGAAGGCAAAGTCCAGGC |
| | | ACAAGAGTGGGACCCCAGCCTCTCACTCTCAGTTCCACTCATCCAACTGGGACCC |
| | | TCACCACGAATCTCATGATCTGATTCGGTTCCCTGTCTCCTCCTCCCGTCACAGAT |
| | | GTGAGCCAGGGCACTGCTCAGCTGTGACCCTAGGTGTTTCTGCCTTGTTGACATG |
| | | GAGAGAGCCCTTTCCCCTGAGAAGGCCTGGCCCCTTCCTGTGCTGAGCCCACAGC |
| | | AGCAGGCTGGGTGTCTTGGTTGTCAGTGGTGGCACCAGGATGGAAGGGCAAGGC |
| | | ACCCAGGGCAGGCCCACAGTCCCGCTGTCCCCCACTTGCACCCTAGCTTGTAGCT |
| | | GCCAACCTCCCAGACAGCCCAGCCCGCTGCTCAGCTCCACATGCATAGTATCAGC |
| | | CCTCCACACCCGACAAAGGGGAACACACCCCCTTGGAAATGGTTCTTTTCCCCCA |
| | | GTCCCAGCTGGAAGCCATGCTGTCTGTTCTGCTGGAGCAGCTGAACATATACATA |
| | | GATGTTGCCCTGCCCTCCCCATCTGCACCCTGTTGAGTTGTAGTTGGATTTGTCTG |
| | | TTTATGCTTGGATTCACCAGAGTGACTATGATAGTGAAAAGAAAAAAAAAAAAA |
| | | AAAAAGGACGCATGTATCTTGAAATGCTTGTAAAGAGGTTTCTAACCCACCCTCA |
| | | CGAGGTGTCTCTCACCCCCACACTGGGACTCGTGTGGCCTGTGTGGTGCCACCCT |
| | | GCTGGGGCCTCCCAAGTTTTGAAAGGCTTTCCTCAGCACCTGGGACCCAACAGAG |
| | | ACCAGCTTCTAGCAGCTAAGGAGGCCGTTCAGCTGTGACGAAGGCCTGAAGCAC |
| | | AGGATTAGGACTGAAGCGATGATGTCCCCTTCCCTACTTCCCCTTGGGGCTCCCT |
| | | GTGTCAGGGCACAGACTAGGTCTTGTGGCTGGTCTGGCTTGCGGCGCGAGGATG |
| | | GTTCTCTCTGGTCATAGCCCGAAGTCTCATGGCAGTCCCAAAGGAGGCTTACAAC |
| | | TCCTGCATCACAAGAAAAAGGAAGCCACTGCCAGCTGGGGGGATCTGCAGCTCC |
| | | CAGAAGCTCCGTGAGCCTCAGCCACCCCTCAGACTGGGTTCCTCTCCAAGCTCGC |
| | | CCTCTGGAGGGGCAGCGCAGCCTCCCACCAAGGGCCCTGCGACCACAGCAGGGA |
| | | TTGGGATGAATTGCCTGTCCTGGATCTGCTCTAGAGGCCCAAGCTGCCTGCCTGA |
| | | GGAAGGATGACTTGACAAGTCAGGAGACACTGTTCCCAAAGCCTTGACCAGAGC |
| | | ACCTCAGCCCGCTGACCTTGCACAAACTCCATCTGCTGCCATGAGAAAAGGGAA |
| | | GCCGCCTTTGCAAAACATTGCTGCCTAAAGAAACTCAGCAGCCTCAGGCCCAATT |
| | | CTGCCACTTCTGGTTTGGGTACAGTTAAAGGCAACCCTGAGGGACTTGGCAGTAG |
| | | AAATCCAGGGCCTCCCCTGGGGCTGGCAGCTTCGTGTGCAGCTAGAGCTTTACCT |
| | | GAAAGGAAGTCTCTGGGCCCAGAACTCTCCACCAAGAGCCTCCCTGCCGTTCGCT |
| | | GAGTCCCAGCAATTCTCCTAAGTTGAAGGGATCTGAGAAGGAGAAGGAAATGTG |
| | | GGGTAGATTTGGTGGTGGTTAGAGATATGCCCCCCTCATTACTGCCAACAGTTTC |
| | | GGCTGCATTTCTTCACGCACCTCGGTTCCTCTTCCTGAAGTTCTTGTGCCCTGCTC |
| | | TTCAGCACCATGGGCCTTCTTATACGGAAGGCTCTGGGATCTCCCCCTTGTGGGG |
| | | CAGGCTCTTGGGGCCAGCCTAAGATCATGGTTTAGGGTGATCAGTGCTGGCAGAT |
| | | AAATTGAAAAGGCACGCTGGCTTGTGATCTTAAATGAGGACAATCCCCCCAGGG |
| | | CTGGGCACTCCTCCCCTCCCCTCACTTCTCCCACCTGCAGAGCCAGTGTCCTTGGG |
| | | TGGGCTAGATAGGATATACTGTATGCCGGCTCCTTCAAGCTGCTGACTCACTTTA |
| | | TCAATAGTTCCATTTAAATTGACTTCAGTGGTGAGACTGTATCCTGTTTGCTATTG |
| | | CTTGTTGTGCTATGGGGGGAGGGGGGAGGAATGTGTAAGATAGTTAACATGGGC |
| | | AAAGGGAGATCTTGGGGTGCAGCACTTAAACTGCCTCGTAACCCTTTTCATGATT |
| | | TCAACCACATTTGCTAGAGGGAGGGAGCAGCCACGGAGTTAGAGGCCCTTGGGG |
| | | TTTCTCTTTTCCACTGACAGGCTTTCCCAGGCAGCTGGCTAGTTCATTCCCTCCCC |
| | | AGCCAGGTGCAGGCGTAGGAATATGGACATCTGGTTGCTTTGGCCTGCTGCCCTC |
| | | TTTCAGGGGTCCTAAGCCCACAATCATGCCTCCCTAAGACCTTGGCATCCTTCCC |
| | | TCTAAGCCGTTGGCACCTCTGTGCCACCTCTCACACTGGCTCCAGACACACAGCC |
| | | TGTGCTTTTGGAGCTGAGATCACTCGCTTCACCCTCCTCATCTTTGTTCTCCAAGT |
| | | AAAGCCACGAGGTCGGGGCGAGGGCAGAGGTGATCACCTGCGTGTCCCATCTAC |
| | | AGACCTGCAGCTTCATAAAACTTCTGATTTCTCTTCAGCTTTGAAAAGGGTTACC |
| | | CTGGGCACTGGCCTAGAGCCTCACCTCCTAATAGACTTAGCCCCATGAGTTTGCC |
| | | ATGTTGAGCAGGACTATTTCTGGCACTTGCAAGTCCCATGATTTCTTCGGTAATTC |
| | | TGAGGGTGGGGGGAGGGACATGAAATCATCTTAGCTTAGCTTTCTGTCTGTGAAT |
| | | GTCTATATAGTGTATTGTGTGTTTTAACAAATGATTTACACTGACTGTTGCTGTAA |
| | | AAGTGAATTTGGAAATAAAGTTATTACTCTGATTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGGTGAACTTTGAACCAGGATGGCTGAGCCCCGC |
| 407 | | CAGGAGTTCGAAGTGATGGAAGATCACGCTGGGACGTACGGGTTGGGGGACAGG |
| | | AAAGATCAGGGGGGCTACACCATGCACCAAGACCAAGAGGGTGACACGGACGC |
| | | TGGCCTGAAAGAATCTCCCCTGCAGACCCCCACTGAGGACGGATCTGAGGAACC |
| | | GGGCTCTGAAACCTCTGATGCTAAGAGCACTCCAACAGCGGAAGCTGAAGAAGC |
| | | AGGCATTGGAGACACCCCCAGCCTGGAAGACGAAGCTGCTGGTCACGTGACCCA |
| | | AGCTCGCATGGTCAGTAAAAGCAAAGACGGGACTGGAAGCGATGACAAAAAAG |
| | | CCAAGGGGGCTGATGGTAAAACGAAGATCGCCACACCGCGGGGAGCAGCCCCTC |
| | | CAGGCCAGAAGGGCCAGGCCAACGCCACCAGGATTCCAGCAAAAACCCCGCCCG |
| | | CTCCAAAGACACCACCCAGCTCTGGTGAACCTCCAAAATCAGGGGATCGCAGCG |
| | | GCTACAGCAGCCCCGGCTCCCCAGGCACTCCCGGCAGCCGCTCCCGCACCCCGTC |
| | | CCTTCCAACCCCACCCACCCGGGAGCCCAAGAAGGTGGCAGTGGTCCGTACTCC |
| | | ACCCAAGTCGCCGTCTTCCGCCAAGAGCCGCCTGCAGACAGCCCCCGTGCCCATG |
| | | CCAGACCTGAAGAATGTCAAGTCCAAGATCGGCTCCACTGAGAACCTGAAGCAC |
| | | CAGCCGGGAGGCGGGAAGGTGCAAATAGTCTACAAACCAGTTGACCTGAGCAAG |
| | | GTTGGAACTGCTGCCATGATTTTGGCCACTTTGCAGACCTGGGACTTTAGGGCTA |
| | | ACCAGTTCTCTTTGTAAGGACTTGTGCCTCTTGGGAGACGTCCACCCGTTTCCAA |
| | | GCCTGGGCCACTGGCATCTCTGGAGTGTGTGGGGGTCTGGGAGGCAGGTCCCGA |
| | | GCCCCCTGTCCTTCCCACGGCCACTGCAGTCACCCCGTCTGCGCCGCTGTGCTGTT |
| | | GTCTGCCGTGAGAGCCCAATCACTGCCTATACCCCTCATCACACGTCACAATGTC |
| | | CCGAATTCCCAGCCTCACCACCCCTTCTCAGTAATGACCCTGGTTGGTTGCAGGA |
| | | GGTACCTACTCCATACTGAGGGTGAAATTAAGGGAAGGCAAAGTCCAGGCACAA |
| | | GAGTGGGACCCCAGCCTCTCACTCTCAGTTCCACTCATCCAACTGGGACCCTCAC |
| | | CACGAATCTCATGATCTGATTCGGTTCCCTGTCTCCTCCTCCCGTCACAGATGTGA |
| | | GCCAGGGCACTGCTCAGCTGTGACCCTAGGTGTTTCTGCCTTGTTGACATGGAGA |
| | | GAGCCCTTTCCCCTGAGAAGGCCTGGCCCCTTCCTGTGCTGAGCCCACAGCAGCA |
| | | GGCTGGGTGTCTTGGTTGTCAGTGGTGGCACCAGGATGGAAGGGCAAGGCACCC |
| | | AGGGCAGGCCCACAGTCCCGCTGTCCCCCACTTGCACCCTAGCTTGTAGCTGCCA |
| | | ACCTCCCAGACAGCCCAGCCCGCTGCTCAGCTCCACATGCATAGTATCAGCCCTC |
| | | CACACCCGACAAAGGGGAACACACCCCCTTGGAAATGGTTCTTTTCCCCCAGTCC |
| | | CAGCTGGAAGCCATGCTGTCTGTTCTGCTGGAGCAGCTGAACATATACATAGATG |
| | | TTGCCCTGCCCTCCCCATCTGCACCCTGTTGAGTTGTAGTTGGATTTGTCTGTTTA |
| | | TGCTTGGATTCACCAGAGTGACTATGATAGTGAAAAGAAAAAAAAAAAAAAAA |
| | | AAGGACGCATGTATCTTGAAATGCTTGTAAAGAGGTTTCTAACCCACCCTCACGA |
| | | GGTGTCTCTCACCCCCACACTGGGACTCGTGTGGCCTGTGTGGTGCCACCCTGCT |
| | | GGGGCCTCCCAAGTTTTGAAAGGCTTTCCTCAGCACCTGGGACCCAACAGAGAC |
| | | CAGCTTCTAGCAGCTAAGGAGGCCGTTCAGCTGTGACGAAGGCCTGAAGCACAG |
| | | GATTAGGACTGAAGCGATGATGTCCCCTTCCCTACTTCCCCTTGGGGCTCCCTGT |
| | | GTCAGGGCACAGACTAGGTCTTGTGGCTGGTCTGGCTTGCGGCGCGAGGATGGTT |
| | | CTCTCTGGTCATAGCCCGAAGTCTCATGGCAGTCCCAAAGGAGGCTTACAACTCC |
| | | TGCATCACAAGAAAAAGGAAGCCACTGCCAGCTGGGGGGATCTGCAGCTCCCAG |
| | | AAGCTCCGTGAGCCTCAGCCACCCCTCAGACTGGGTTCCTCTCCAAGCTCGCCCT |
| | | CTGGAGGGGCAGCGCAGCCTCCCACCAAGGGCCCTGCGACCACAGCAGGGATTG |
| | | GGATGAATTGCCTGTCCTGGATCTGCTCTAGAGGCCCAAGCTGCCTGCCTGAGGA |
| | | AGGATGACTTGACAAGTCAGGAGACACTGTTCCCAAAGCCTTGACCAGAGCACC |
| | | TCAGCCCGCTGACCTTGCACAAACTCCATCTGCTGCCATGAGAAAAGGGAAGCC |
| | | GCCTTTGCAAAACATTGCTGCCTAAAGAAACTCAGCAGCCTCAGGCCCAATTCTG |
| | | CCACTTCTGGTTTGGGTACAGTTAAAGGCAACCCTGAGGGACTTGGCAGTAGAA |
| | | ATCCAGGGCCTCCCCTGGGGCTGGCAGCTTCGTGTGCAGCTAGAGCTTTACCTGA |
| | | AAGGAAGTCTCTGGGCCCAGAACTCTCCACCAAGAGCCTCCCTGCCGTTCGCTGA |
| | | GTCCCAGCAATTCTCCTAAGTTGAAGGGATCTGAGAAGGAGAAGGAAATGTGGG |
| | | GTAGATTTGGTGGTGGTTAGAGATATGCCCCCCTCATTACTGCCAACAGTTTCGG |
| | | CTGCATTTCTTCACGCACCTCGGTTCCTCTTCCTGAAGTTCTTGTGCCCTGCTCTTC |
| | | AGCACCATGGGCCTTCTTATACGGAAGGCTCTGGGATCTCCCCCTTGTGGGGCAG |
| | | GCTCTTGGGGCCAGCCTAAGATCATGGTTTAGGGTGATCAGTGCTGGCAGATAA |
| | | ATTGAAAAGGCACGCTGGCTTGTGATCTTAAATGAGGACAATCCCCCCAGGGCT |
| | | GGGCACTCCTCCCCTCCCCTCACTTCTCCCACCTGCAGAGCCAGTGTCCTTGGGT |
| | | GGGCTAGATAGGATATACTGTATGCCGGCTCCTTCAAGCTGCTGACTCACTTTAT |
| | | CAATAGTTCCATTTAAATTGACTTCAGTGGTGAGACTGTATCCTGTTTGCTATTGC |
| | | TTGTTGTGCTATGGGGGGAGGGGGGAGGAATGTGTAAGATAGTTAACATGGGCA |
| | | AAGGGAGATCTTGGGGTGCAGCACTTAAACTGCCTCGTAACCCTTTTCATGATTT |
| | | CAACCACATTTGCTAGAGGGAGGGAGCAGCCACGGAGTTAGAGGCCCTTGGGGT |
| | | TTCTCTTTTCCACTGACAGGCTTTCCCAGGCAGCTGGCTAGTTCATTCCCTCCCCA |
| | | GCCAGGTGCAGGCGTAGGAATATGGACATCTGGTTGCTTTGGCCTGCTGCCCTCT |
| | | TTCAGGGGTCCTAAGCCCACAATCATGCCTCCCTAAGACCTTGGCATCCTTCCCT |
| | | CTAAGCCGTTGGCACCTCTGTGCCACCTCTCACACTGGCTCCAGACACACAGCCT |
| | | GTGCTTTTGGAGCTGAGATCACTCGCTTCACCCTCCTCATCTTTGTTCTCCAAGTA |
| | | AAGCCACGAGGTCGGGGCGAGGGCAGAGGTGATCACCTGCGTGTCCCATCTACA |
| | | GACCTGCAGCTTCATAAAACTTCTGATTTCTCTTCAGCTTTGAAAAGGGTTACCCT |
| | | GGGCACTGGCCTAGAGCCTCACCTCCTAATAGACTTAGCCCCATGAGTTTGCCAT |
| | | GTTGAGCAGGACTATTTCTGGCACTTGCAAGTCCCATGATTTCTTCGGTAATTCTG |
| | | AGGGTGGGGGGAGGGACATGAAATCATCTTAGCTTAGCTTTCTGTCTGTGAATGT |
| | | CTATATAGTGTATTGTGTGTTTTAACAAATGATTTACACTGACTGTTGCTGTAAAA |
| | | GTGAATTTGGAAATAAAGTTATTACTCTGATTAAA |
| SEQ | MAPT | GCAGTCACCGCCACCCACCAGCTCCGGCACCAACAGCAGCGCCGCTGCCACCGC |
| ID | | CCACCTTCTGCCGCCGCCACCACAGCCACCTTCTCCTCCTCCGCTGTCCTCTCCCG |
| NO: | | TCCTCGCCTCTGTCGACTATCAGACAGGCTCTGCTGATGCTGTCCCTCTCCTGTTC |
| 408 | | AGTCGTGCCCTCACCGTTAAAGAGAAAGAGCAAACTGCTGGGCAGCAGCATTGA |
| | | TTTTTTTAATGAAGTGGAAAGAGAGCTGGGAATAACAAGTCGGGCCCACCTCAC |
| | | CTGCCTCACCTGGTGAACTTTGAACCAGGATGGCTGAGCCCCGCCAGGAGTTCGA |
| | | AGTGATGGAAGATCACGCTGGGACGTACGGGTTGGGGGACAGGAAAGATCAGG |
| | | GGGGCTACACCATGCACCAAGACCAAGAGGGTGACACGGACGCTGGCCTGAAA |
| | | GCTGAAGAAGCAGGCATTGGAGACACCCCCAGCCTGGAAGACGAAGCTGCTGGT |
| | | CACGTGACCCAAGCTCGCATGGTCAGTAAAAGCAAAGACGGGACTGGAAGCGAT |
| | | GACAAAAAAGCCAAGGGGGCTGATGGTAAAACGAAGATCGCCACACCGCGGGG |
| | | AGCAGCCCCTCCAGGCCAGAAGGGCCAGGCCAACGCCACCAGGATTCCAGCAAA |
| | | AACCCCGCCCGCTCCAAAGACACCACCCAGCTCTGGTGAACCTCCAAAATCAGG |
| | | GGATCGCAGCGGCTACAGCAGCCCCGGCTCCCCAGGCACTCCCGGCAGCCGCTC |
| | | CCGCACCCCGTCCCTTCCAACCCCACCCACCCGGGAGCCCAAGAAGGTGGCAGT |
| | | GGTCCGTACTCCACCCAAGTCGCCGTCTTCCGCCAAGAGCCGCCTGCAGACAGCC |
| | | CCCGTGCCCATGCCAGACCTGAAGAATGTCAAGTCCAAGATCGGCTCCACTGAG |
| | | AACCTGAAGCACCAGCCGGGAGGCGGGAAGGTGCAAATAGTCTACAAACCAGTT |
| | | GACCTGAGCAAGGTGACCTCCAAGTGTGGCTCATTAGGCAACATCCATCATAAA |
| | | CCAGGAGGTGGCCAGGTGGAAGTAAAATCTGAGAAGCTTGACTTCAAGGACAGA |
| | | GTCCAGTCGAAGATTGGGTCCCTGGACAATATCACCCACGTCCCTGGCGGAGGA |
| | | AATAAAAAGATTGAAACCCACAAGCTGACCTTCCGCGAGAACGCCAAAGCCAAG |
| | | ACAGACCACGGGGCGGAGATCGTGTACAAGTCGCCAGTGGTGTCTGGGGACACG |
| | | TCTCCACGGCATCTCAGCAATGTCTCCTCCACCGGCAGCATCGACATGGTAGACT |
| | | CGCCCCAGCTCGCCACGCTAGCTGACGAGGTGTCTGCCTCCCTGGCCAAGCAGG |
| | | GTTTGTGATCAGGCCCCTGGGGCGGTCAATAATTGTGGAGAGGAGAGAATGAGA |
| | | GAGTGTGGAAAAAAAAAGAATAATGACCCGGCCCCCGCCCTCTGCCCCCAGCTG |
| | | CTCCTCGCAGTTCGGTTAATTGGTTAATCACTTAACCTGCTTTTGTCACTCGGCTT |
| | | TGGCTCGGGACTTCAAAATCAGTGATGGGAGTAAGAGCAAATTTCATCTTTCCAA |
| | | ATTGATGGGTGGGCTAGTAATAAAATATTTAAAAAAAAACATTCAAAAACATGG |
| | | CCACATCCAACATTTCCTCAGGCAATTCCTTTTGATTCTTTTTTCTTCCCCCTCCAT |
| | | GTAGAAGAGGGAGAAGGAGAGGCTCTGAAAGCTGCTTCTGGGGGATTTCAAGGG |
| | | ACTGGGGGTGCCAACCACCTCTGGCCCTGTTGTGGGGGTGTCACAGAGGCAGTG |
| | | GCAGCAACAAAGGATTTGAAACTTGGTGTGTTCGTGGAGCCACAGGCAGACGAT |
| | | GTCAACCTTGTGTGAGTGTGACGGGGGTTGGGGTGGGGCGGGAGGCCACGGGGG |
| | | AGGCCGAGGCAGGGGCTGGGCAGAGGGGAGAGGAAGCACAAGAAGTGGGAGTG |
| | | GGAGAGGAAGCCACGTGCTGGAGAGTAGACATCCCCCTCCTTGCCGCTGGGAGA |
| | | GCCAAGGCCTATGCCACCTGCAGCGTCTGAGCGGCCGCCTGTCCTTGGTGGCCGG |
| | | GGGTGGGGGCCTGCTGTGGGTCAGTGTGCCACCCTCTGCAGGGCAGCCTGTGGG |
| | | AGAAGGGACAGCGGGTAAAAAGAGAAGGCAAGCTGGCAGGAGGGTGGCACTTC |
| | | GTGGATGACCTCCTTAGAAAAGACTGACCTTGATGTCTTGAGAGCGCTGGCCTCT |
| | | TCCTCCCTCCCTGCAGGGTAGGGGGCCTGAGTTGAGGGGCTTCCCTCTGCTCCAC |
| | | AGAAACCCTGTTTTATTGAGTTCTGAAGGTTGGAACTGCTGCCATGATTTTGGCC |
| | | ACTTTGCAGACCTGGGACTTTAGGGCTAACCAGTTCTCTTTGTAAGGACTTGTGC |
| | | CTCTTGGGAGACGTCCACCCGTTTCCAAGCCTGGGCCACTGGCATCTCTGGAGTG |
| | | TGTGGGGGTCTGGGAGGCAGGTCCCGAGCCCCCTGTCCTTCCCACGGCCACTGCA |
| | | GTCACCCCGTCTGCGCCGCTGTGCTGTTGTCTGCCGTGAGAGCCCAATCACTGCC |
| | | TATACCCCTCATCACACGTCACAATGTCCCGAATTCCCAGCCTCACCACCCCTTCT |
| | | CAGTAATGACCCTGGTTGGTTGCAGGAGGTACCTACTCCATACTGAGGGTGAAAT |
| | | TAAGGGAAGGCAAAGTCCAGGCACAAGAGTGGGACCCCAGCCTCTCACTCTCAG |
| | | TTCCACTCATCCAACTGGGACCCTCACCACGAATCTCATGATCTGATTCGGTTCC |
| | | CTGTCTCCTCCTCCCGTCACAGATGTGAGCCAGGGCACTGCTCAGCTGTGACCCT |
| | | AGGTGTTTCTGCCTTGTTGACATGGAGAGAGCCCTTTCCCCTGAGAAGGCCTGGC |
| | | CCCTTCCTGTGCTGAGCCCACAGCAGCAGGCTGGGTGTCTTGGTTGTCAGTGGTG |
| | | GCACCAGGATGGAAGGGCAAGGCACCCAGGGCAGGCCCACAGTCCCGCTGTCCC |
| | | CCACTTGCACCCTAGCTTGTAGCTGCCAACCTCCCAGACAGCCCAGCCCGCTGCT |
| | | CAGCTCCACATGCATAGTATCAGCCCTCCACACCCGACAAAGGGGAACACACCC |
| | | CCTTGGAAATGGTTCTTTTCCCCCAGTCCCAGCTGGAAGCCATGCTGTCTGTTCTG |
| | | CTGGAGCAGCTGAACATATACATAGATGTTGCCCTGCCCTCCCCATCTGCACCCT |
| | | GTTGAGTTGTAGTTGGATTTGTCTGTTTATGCTTGGATTCACCAGAGTGACTATGA |
| | | TAGTGAAAAGAAAAAAAAAAAAAAAAAAGGACGCATGTATCTTGAAATGCTTGT |
| | | AAAGAGGTTTCTAACCCACCCTCACGAGGTGTCTCTCACCCCCACACTGGGACTC |
| | | GTGTGGCCTGTGTGGTGCCACCCTGCTGGGGCCTCCCAAGTTTTGAAAGGCTTTC |
| | | CTCAGCACCTGGGACCCAACAGAGACCAGCTTCTAGCAGCTAAGGAGGCCGTTC |
| | | AGCTGTGACGAAGGCCTGAAGCACAGGATTAGGACTGAAGCGATGATGTCCCCT |
| | | TCCCTACTTCCCCTTGGGGCTCCCTGTGTCAGGGCACAGACTAGGTCTTGTGGCT |
| | | GGTCTGGCTTGCGGCGCGAGGATGGTTCTCTCTGGTCATAGCCCGAAGTCTCATG |
| | | GCAGTCCCAAAGGAGGCTTACAACTCCTGCATCACAAGAAAAAGGAAGCCACTG |
| | | CCAGCTGGGGGGATCTGCAGCTCCCAGAAGCTCCGTGAGCCTCAGCCACCCCTC |
| | | AGACTGGGTTCCTCTCCAAGCTCGCCCTCTGGAGGGGCAGCGCAGCCTCCCACCA |
| | | AGGGCCCTGCGACCACAGCAGGGATTGGGATGAATTGCCTGTCCTGGATCTGCTC |
| | | TAGAGGCCCAAGCTGCCTGCCTGAGGAAGGATGACTTGACAAGTCAGGAGACAC |
| | | TGTTCCCAAAGCCTTGACCAGAGCACCTCAGCCCGCTGACCTTGCACAAACTCCA |
| | | TCTGCTGCCATGAGAAAAGGGAAGCCGCCTTTGCAAAACATTGCTGCCTAAAGA |
| | | AACTCAGCAGCCTCAGGCCCAATTCTGCCACTTCTGGTTTGGGTACAGTTAAAGG |
| | | CAACCCTGAGGGACTTGGCAGTAGAAATCCAGGGCCTCCCCTGGGGCTGGCAGC |
| | | TTCGTGTGCAGCTAGAGCTTTACCTGAAAGGAAGTCTCTGGGCCCAGAACTCTCC |
| | | ACCAAGAGCCTCCCTGCCGTTCGCTGAGTCCCAGCAATTCTCCTAAGTTGAAGGG |
| | | ATCTGAGAAGGAGAAGGAAATGTGGGGTAGATTTGGTGGTGGTTAGAGATATGC |
| | | CCCCCTCATTACTGCCAACAGTTTCGGCTGCATTTCTTCACGCACCTCGGTTCCTC |
| | | TTCCTGAAGTTCTTGTGCCCTGCTCTTCAGCACCATGGGCCTTCTTATACGGAAGG |
| | | CTCTGGGATCTCCCCCTTGTGGGGCAGGCTCTTGGGGCCAGCCTAAGATCATGGT |
| | | TTAGGGTGATCAGTGCTGGCAGATAAATTGAAAAGGCACGCTGGCTTGTGATCTT |
| | | AAATGAGGACAATCCCCCCAGGGCTGGGCACTCCTCCCCTCCCCTCACTTCTCCC |
| | | ACCTGCAGAGCCAGTGTCCTTGGGTGGGCTAGATAGGATATACTGTATGCCGGCT |
| | | CCTTCAAGCTGCTGACTCACTTTATCAATAGTTCCATTTAAATTGACTTCAGTGGT |
| | | GAGACTGTATCCTGTTTGCTATTGCTTGTTGTGCTATGGGGGGGGGGGGAGGAA |
| | | TGTGTAAGATAGTTAACATGGGCAAAGGGAGATCTTGGGGTGCAGCACTTAAAC |
| | | TGCCTCGTAACCCTTTTCATGATTTCAACCACATTTGCTAGAGGGAGGGAGCAGC |
| | | CACGGAGTTAGAGGCCCTTGGGGTTTCTCTTTTCCACTGACAGGCTTTCCCAGGC |
| | | AGCTGGCTAGTTCATTCCCTCCCCAGCCAGGTGCAGGCGTAGGAATATGGACATC |
| | | TGGTTGCTTTGGCCTGCTGCCCTCTTTCAGGGGTCCTAAGCCCACAATCATGCCTC |
| | | CCTAAGACCTTGGCATCCTTCCCTCTAAGCCGTTGGCACCTCTGTGCCACCTCTCA |
| | | CACTGGCTCCAGACACACAGCCTGTGCTTTTGGAGCTGAGATCACTCGCTTCACC |
| | | CTCCTCATCTTTGTTCTCCAAGTAAAGCCACGAGGTCGGGGCGAGGGCAGAGGT |
| | | GATCACCTGCGTGTCCCATCTACAGACCTGCAGCTTCATAAAACTTCTGATTTCTC |
| | | TTCAGCTTTGAAAAGGGTTACCCTGGGCACTGGCCTAGAGCCTCACCTCCTAATA |
| | | GACTTAGCCCCATGAGTTTGCCATGTTGAGCAGGACTATTTCTGGCACTTGCAAG |
| | | TCCCATGATTTCTTCGGTAATTCTGAGGGTGGGGGGAGGGACATGAAATCATCTT |
| | | AGCTTAGCTTTCTGTCTGTGAATGTCTATATAGTGTATTGTGTGTTTTAACAAATG |
| | | ATTTACACTGACTGTTGCTGTAAAAGTGAATTTGGAAATAAAGTTATTACTCTGA |
| | | TTAAA |
| SEQ | BCL2 | ACCCCTCGCCGCACCACACACAGCGCGGGCTTCTAGCGCTCGGCACCGGCGGGC |
| ID | | CAGGCGCGTCCTGCCTTCATTTATCCAGCAGCTTTTCGGAAAATGCATTTGCTGTT |
| NO: | | CGGAGTTTAATCAGAAGAGGATTCCTGCCTCCGTCCCCGGCTCCTTCATCGTCCC |
| 409 | | CTCTCCCCTGTCTCTCTCCTGGGGAGGCGTGAAGCGGTCCCGTGGATAGAGATTC |
| | | ATGCCTGTGCCCGCGCGTGTGTGCGCGCGTGTAAATTGCCGAGAAGGGGAAAAC |
| | | ATCACAGGACTTCTGCGAATACCGGACTGAAAATTGTAATTCATCTGCCGCCGCC |
| | | GCTGCCTTTTTTTTTTCTCGAGCTCTTGAGATCTCCGGTTGGGATTCCTGCGGATT |
| | | GACATTTCTGTGAAGCAGAAGTCTGGGAATCGATCTGGAAATCCTCCTAATTTTT |
| | | ACTCCCTCTCCCCGCGACTCCTGATTCATTGGGAAGTTTCAAATCAGCTATAACT |
| | | GGAGAGTGCTGAAGATTGATGGGATCGTTGCCTTATGCATTTGTTTTGGTTTTAC |
| | | AAAAAGGAAACTTGACAGAGGATCATGCTGTACTTAAAAAATACAACATCACAG |
| | | AGGAAGTAGACTGATATTAACAATACTTACTAATAATAACGTGCCTCATGAAAT |
| | | AAAGATCCGAAAGGAATTGGAATAAAAATTTCCTGCATCTCATGCCAAGGGGGA |
| | | AACACCAGAATCAAGTGTTCCGCGTGATTGAAGACACCCCCTCGTCCAAGAATG |
| | | CAAAGCACATCCAATAAAATAGCTGGATTATAACTCCTCTTCTTTCTCTGGGGGC |
| | | CGTGGGGTGGGAGCTGGGGCGAGAGGTGCCGTTGGCCCCCGTTGCTTTTCCTCTG |
| | | GGAAGGATGGCGCACGCTGGGAGAACAGGGTACGATAACCGGGAGATAGTGAT |
| | | GAAGTACATCCATTATAAGCTGTCGCAGAGGGGCTACGAGTGGGATGCGGGAGA |
| | | TGTGGGCGCCGCGCCCCCGGGGGCCGCCCCCGCACCGGGCATCTTCTCCTCCCAG |
| | | CCCGGGCACACGCCCCATCCAGCCGCATCCCGGGACCCGGTCGCCAGGACCTCG |
| | | CCGCTGCAGACCCCGGCTGCCCCCGGCGCCGCCGCGGGGCCTGCGCTCAGCCCG |
| | | GTGCCACCTGTGGTCCACCTGACCCTCCGCCAGGCCGGCGACGACTTCTCCCGCC |
| | | GCTACCGCCGCGACTTCGCCGAGATGTCCAGCCAGCTGCACCTGACGCCCTTCAC |
| | | CGCGCGGGGACGCTTTGCCACGGTGGTGGAGGAGCTCTTCAGGGACGGGGTGAA |
| | | CTGGGGGAGGATTGTGGCCTTCTTTGAGTTCGGTGGGGTCATGTGTGTGGAGAGC |
| | | GTCAACCGGGAGATGTCGCCCCTGGTGGACAACATCGCCCTGTGGATGACTGAG |
| | | TACCTGAACCGGCACCTGCACACCTGGATCCAGGATAACGGAGGCTGGGATGCC |
| | | TTTGTGGAACTGTACGGCCCCAGCATGCGGCCTCTGTTTGATTTCTCCTGGCTGTC |
| | | TCTGAAGACTCTGCTCAGTTTGGCCCTGGTGGGAGCTTGCATCACCCTGGGTGCC |
| | | TATCTGGGCCACAAGTGAAGTCAACATGCCTGCCCCAAACAAATATGCAAAAGG |
| | | TTCACTAAAGCAGTAGAAATAATATGCATTGTCAGTGATGTACCATGAAACAAA |
| | | GCTGCAGGCTGTTTAAGAAAAAATAACACACATATAAACATCACACACACAGAC |
| | | AGACACACACACACACAACAATTAACAGTCTTCAGGCAAAACGTCGAATCAGCT |
| | | ATTTACTGCCAAAGGGAAATATCATTTATTTTTTACATTATTAAGAAAAAAAGAT |
| | | TTATTTATTTAAGACAGTCCCATCAAAACTCCTGTCTTTGGAAATCCGACCACTA |
| | | ATTGCCAAGCACCGCTTCGTGTGGCTCCACCTGGATGTTCTGTGCCTGTAAACAT |
| | | AGATTCGCTTTCCATGTTGTTGGCCGGATCACCATCTGAAGAGCAGACGGATGGA |
| | | AAAAGGACCTGATCATTGGGGAAGCTGGCTTTCTGGCTGCTGGAGGCTGGGGAG |
| | | AAGGTGTTCATTCACTTGCATTTCTTTGCCCTGGGGGCTGTGATATTAACAGAGG |
| | | GAGGGTTCCTGTGGGGGGAAGTCCATGCCTCCCTGGCCTGAAGAAGAGACTCTTT |
| | | GCATATGACTCACATGATGCATACCTGGTGGGAGGAAAAGAGTTGGGAACTTCA |
| | | GATGGACCTAGTACCCACTGAGATTTCCACGCCGAAGGACAGCGATGGGAAAAA |
| | | TGCCCTTAAATCATAGGAAAGTATTTTTTTAAGCTACCAATTGTGCCGAGAAAAG |
| | | CATTTTAGCAATTTATACAATATCATCCAGTACCTTAAGCCCTGATTGTGTATATT |
| | | CATATATTTTGGATACGCACCCCCCAACTCCCAATACTGGCTCTGTCTGAGTAAG |
| | | AAACAGAATCCTCTGGAACTTGAGGAAGTGAACATTTCGGTGACTTCCGCATCA |
| | | GGAAGGCTAGAGTTACCCAGAGCATCAGGCCGCCACAAGTGCCTGCTTTTAGGA |
| | | GACCGAAGTCCGCAGAACCTGCCTGTGTCCCAGCTTGGAGGCCTGGTCCTGGAA |
| | | CTGAGCCGGGGCCCTCACTGGCCTCCTCCAGGGATGATCAACAGGGCAGTGTGG |
| | | TCTCCGAATGTCTGGAAGCTGATGGAGCTCAGAATTCCACTGTCAAGAAAGAGC |
| | | AGTAGAGGGGTGTGGCTGGGCCTGTCACCCTGGGGCCCTCCAGGTAGGCCCGTTT |
| | | TCACGTGGAGCATGGGAGCCACGACCCTTCTTAAGACATGTATCACTGTAGAGG |
| | | GAAGGAACAGAGGCCCTGGGCCCTTCCTATCAGAAGGACATGGTGAAGGCTGGG |
| | | AACGTGAGGAGAGGCAATGGCCACGGCCCATTTTGGCTGTAGCACATGGCACGT |
| | | TGGCTGTGTGGCCTTGGCCCACCTGTGAGTTTAAAGCAAGGCTTTAAATGACTTT |
| | | GGAGAGGGTCACAAATCCTAAAAGAAGCATTGAAGTGAGGTGTCATGGATTAAT |
| | | TGACCCCTGTCTATGGAATTACATGTAAAACATTATCTTGTCACTGTAGTTTGGTT |
| | | TTATTTGAAAACCTGACAAAAAAAAAGTTCCAGGTGTGGAATATGGGGGTTATC |
| | | TGTACATCCTGGGGCATTAAAAAAAAAATCAATGGTGGGGAACTATAAAGAAGT |
| | | AACAAAAGAAGTGACATCTTCAGCAAATAAACTAGGAAATTTTTTTTTCTTCCAG |
| | | TTTAGAATCAGCCTTGAAACATTGATGGAATAACTCTGTGGCATTATTGCATTAT |
| | | ATACCATTTATCTGTATTAACTTTGGAATGTACTCTGTTCAATGTTTAATGCTGTG |
| | | GTTGATATTTCGAAAGCTGCTTTAAAAAAATACATGCATCTCAGCGTTTTTTTGTT |
| | | TTTAATTGTATTTAGTTATGGCCTATACACTATTTGTGAGCAAAGGTGATCGTTTT |
| | | CTGTTTGAGATTTTTATCTCTTGATTCTTCAAAAGCATTCTGAGAAGGTGAGATA |
| | | AGCCCTGAGTCTCAGCTACCTAAGAAAAACCTGGATGTCACTGGCCACTGAGGA |
| | | GCTTTGTTTCAACCAAGTCATGTGCATTTCCACGTCAACAGAATTGTTTATTGTGA |
| | | CAGTTATATCTGTTGTCCCTTTGACCTTGTTTCTTGAAGGTTTCCTCGTCCCTGGG |
| | | CAATTCCGCATTTAATTCATGGTATTCAGGATTACATGCATGTTTGGTTAAACCC |
| | | ATGAGATTCATTCAGTTAAAAATCCAGATGGCAAATGACCAGCAGATTCAAATC |
| | | TATGGTGGTTTGACCTTTAGAGAGTTGCTTTACGTGGCCTGTTTCAACACAGACC |
| | | CACCCAGAGCCCTCCTGCCCTCCTTCCGCGGGGGCTTTCTCATGGCTGTCCTTCAG |
| | | GGTCTTCCTGAAATGCAGTGGTGCTTACGCTCCACCAAGAAAGCAGGAAACCTG |
| | | TGGTATGAAGCCAGACCTCCCCGGCGGGCCTCAGGGAACAGAATGATCAGACCT |
| | | TTGAATGATTCTAATTTTTAAGCAAAATATTATTTTATGAAAGGTTTACATTGTCA |
| | | AAGTGATGAATATGGAATATCCAATCCTGTGCTGCTATCCTGCCAAAATCATTTT |
| | | AATGGAGTCAGTTTGCAGTATGCTCCACGTGGTAAGATCCTCCAAGCTGCTTTAG |
| | | AAGTAACAATGAAGAACGTGGACGTTTTTAATATAAAGCCTGTTTTGTCTTTTGT |
| | | TGTTGTTCAAACGGGATTCACAGAGTATTTGAAAAATGTATATATATTAAGAGGT |
| | | CACGGGGGCTAATTGCTGGCTGGCTGCCTTTTGCTGTGGGGTTTTGTTACCTGGTT |
| | | TTAATAACAGTAAATGTGCCCAGCCTCTTGGCCCCAGAACTGTACAGTATTGTGG |
| | | CTGCACTTGCTCTAAGAGTAGTTGATGTTGCATTTTCCTTATTGTTAAAAACATGT |
| | | TAGAAGCAATGAATGTATATAAAAGCCTCAACTAGTCATTTTTTTCTCCTCTTCTT |
| | | TTTTTTCATTATATCTAATTATTTTGCAGTTGGGCAACAGAGAACCATCCCTATTT |
| | | TGTATTGAAGAGGGATTCACATCTGCATCTTAACTGCTCTTTATGAATGAAAAAA |
| | | CAGTCCTCTGTATGTACTCCTCTTTACACTGGCCAGGGTCAGAGTTAAATAGAGT |
| | | ATATGCACTTTCCAAATTGGGGACAAGGGCTCTAAAAAAAGCCCCAAAAGGAGA |
| | | AGAACATCTGAGAACCTCCTCGGCCCTCCCAGTCCCTCGCTGCACAAATACTCCG |
| | | CAAGAGAGGCCAGAATGACAGCTGACAGGGTCTATGGCCATCGGGTCGTCTCCG |
| | | AAGATTTGGCAGGGGCAGAAAACTCTGGCAGGCTTAAGATTTGGAATAAAGTCA |
| | | CAGAATTAAGGAAGCACCTCAATTTAGTTCAAACAAGACGCCAACATTCTCTCCA |
| | | CAGCTCACTTACCTCTCTGTGTTCAGATGTGGCCTTCCATTTATATGTGATCTTTG |
| | | TTTTATTAGTAAATGCTTATCATCTAAAGATGTAGCTCTGGCCCAGTGGGAAAAA |
| | | TTAGGAAGTGATTATAAATCGAGAGGAGTTATAATAATCAAGATTAAATGTAAA |
| | | TAATCAGGGCAATCCCAACACATGTCTAGCTTTCACCTCCAGGATCTATTGAGTG |
| | | AACAGAATTGCAAATAGTCTCTATTTGTAATTGAACTTATCCTAAAACAAATAGT |
| | | TTATAAATGTGAACTTAAACTCTAATTAATTCCAACTGTACTTTTAAGGCAGTGG |
| | | CTGTTTTTAGACTTTCTTATCACTTATAGTTAGTAATGTACACCTACTCTATCAGA |
| | | GAAAAACAGGAAAGGCTCGAAATACAAGCCATTCTAAGGAAATTAGGGAGTCA |
| | | GTTGAAATTCTATTCTGATCTTATTCTGTGGTGTCTTTTGCAGCCCAGACAAATGT |
| | | GGTTACACACTTTTTAAGAAATACAATTCTACATTGTCAAGCTTATGAAGGTTCC |
| | | AATCAGATCTTTATTGTTATTCAATTTGGATCTTTCAGGGATTTTTTTTTTAAATTA |
| | | TTATGGGACAAAGGACATTTGTTGGAGGGGTGGGAGGGAGGAAGAATTTTTAAA |
| | | TGTAAAACATTCCCAAGTTTGGATCAGGGAGTTGGAAGTTTTCAGAATAACCAG |
| | | AACTAAGGGTATGAAGGACCTGTATTGGGGTCGATGTGATGCCTCTGCGAAGAA |
| | | CCTTGTGTGACAAATGAGAAACATTTTGAAGTTTGTGGTACGACCTTTAGATTCC |
| | | AGAGACATCAGCATGGCTCAAAGTGCAGCTCCGTTTGGCAGTGCAATGGTATAA |
| | | ATTTCAAGCTGGATATGTCTAATGGGTATTTAAACAATAAATGTGCAGTTTTAAC |
| | | TAACAGGATATTTAATGACAACCTTCTGGTTGGTAGGGACATCTGTTTCTAAATG |
| | | TTTATTATGTACAATACAGAAAAAAATTTTATAAAATTAAGCAATGTGAAACTGA |
| | | ATTGGAGAGTGATAATACAAGTCCTTTAGTCTTACCCAGTGAATCATTCTGTTCC |
| | | ATGTCTTTGGACAACCATGACCTTGGACAATCATGAAATATGCATCTCACTGGAT |
| | | GCAAAGAAAATCAGATGGAGCATGAATGGTACTGTACCGGTTCATCTGGACTGC |
| | | CCCAGAAAAATAACTTCAAGCAAACATCCTATCAACAACAAGGTTGTTCTGCAT |
| | | ACCAAGCTGAGCACAGAAGATGGGAACACTGGTGGAGGATGGAAAGGCTCGCT |
| | | CAATCAAGAAAATTCTGAGACTATTAATAAATAAGACTGTAGTGTAGATACTGA |
| | | GTAAATCCATGCACCTAAACCTTTTGGAAAATCTGCCGTGGGCCCTCCAGATAGC |
| | | TCATTTCATTAAGTTTTTCCCTCCAAGGTAGAATTTGCAAGAGTGACAGTGGATT |
| | | GCATTTCTTTTGGGGAAGCTTTCTTTTGGTGGTTTTGTTTATTATACCTTCTTAAGT |
| | | TTTCAACCAAGGTTTGCTTTTGTTTTGAGTTACTGGGGTTATTTTTGTTTTAAATA |
| | | AAAATAAGTGTACAATAAGTGTTTTTGTATTGAAAGCTTTTGTTATCAAGATTTTC |
| | | ATACTTTTACCTTCCATGGCTCTTTTTAAGATTGATACTTTTAAGAGGTGGCTGAT |
| | | ATTCTGCAACACTGTACACATAAAAAATACGGTAAGGATACTTTACATGGTTAAG |
| | | GTAAAGTAAGTCTCCAGTTGGCCACCATTAGCTATAATGGCACTTTGTTTGTGTT |
| | | GTTGGAAAAAGTCACATTGCCATTAAACTTTCCTTGTCTGTCTAGTTAATATTGTG |
| | | AAGAAAAATAAAGTACAGTGTGAGATACTG |
| SEQ | BCL2 | ACCCCTCGCCGCACCACACACAGCGCGGGCTTCTAGCGCTCGGCACCGGCGGGC |
| ID | | CAGGCGCGTCCTGCCTTCATTTATCCAGCAGCTTTTCGGAAAATGCATTTGCTGTT |
| NO: | | CGGAGTTTAATCAGAAGAGGATTCCTGCCTCCGTCCCCGGCTCCTTCATCGTCCC |
| 410 | | CTCTCCCCTGTCTCTCTCCTGGGGAGGCGTGAAGCGGTCCCGTGGATAGAGATTC |
| | | ATGCCTGTGCCCGCGCGTGTGTGCGCGCGTGTAAATTGCCGAGAAGGGGAAAAC |
| | | ATCACAGGACTTCTGCGAATACCGGACTGAAAATTGTAATTCATCTGCCGCCGCC |
| | | GCTGCCTTTTTTTTTTCTCGAGCTCTTGAGATCTCCGGTTGGGATTCCTGCGGATT |
| | | GACATTTCTGTGAAGCAGAAGTCTGGGAATCGATCTGGAAATCCTCCTAATTTTT |
| | | ACTCCCTCTCCCCGCGACTCCTGATTCATTGGGAAGTTTCAAATCAGCTATAACT |
| | | GGAGAGTGCTGAAGATTGATGGGATCGTTGCCTTATGCATTTGTTTTGGTTTTAC |
| | | AAAAAGGAAACTTGACAGAGGATCATGCTGTACTTAAAAAATACAACATCACAG |
| | | AGGAAGTAGACTGATATTAACAATACTTACTAATAATAACGTGCCTCATGAAAT |
| | | AAAGATCCGAAAGGAATTGGAATAAAAATTTCCTGCATCTCATGCCAAGGGGGA |
| | | AACACCAGAATCAAGTGTTCCGCGTGATTGAAGACACCCCCTCGTCCAAGAATG |
| | | CAAAGCACATCCAATAAAATAGCTGGATTATAACTCCTCTTCTTTCTCTGGGGGC |
| | | CGTGGGGTGGGAGCTGGGGCGAGAGGTGCCGTTGGCCCCCGTTGCTTTTCCTCTG |
| | | GGAAGGATGGCGCACGCTGGGAGAACAGGGTACGATAACCGGGAGATAGTGAT |
| | | GAAGTACATCCATTATAAGCTGTCGCAGAGGGGCTACGAGTGGGATGCGGGAGA |
| | | TGTGGGCGCCGCGCCCCCGGGGGCCGCCCCCGCACCGGGCATCTTCTCCTCCCAG |
| | | CCCGGGCACACGCCCCATCCAGCCGCATCCCGGGACCCGGTCGCCAGGACCTCG |
| | | CCGCTGCAGACCCCGGCTGCCCCCGGCGCCGCCGCGGGGCCTGCGCTCAGCCCG |
| | | GTGCCACCTGTGGTCCACCTGACCCTCCGCCAGGCCGGCGACGACTTCTCCCGCC |
| | | GCTACCGCCGCGACTTCGCCGAGATGTCCAGCCAGCTGCACCTGACGCCCTTCAC |
| | | CGCGCGGGGACGCTTTGCCACGGTGGTGGAGGAGCTCTTCAGGGACGGGGTGAA |
| | | CTGGGGGAGGATTGTGGCCTTCTTTGAGTTCGGTGGGGTCATGTGTGTGGAGAGC |
| | | GTCAACCGGGAGATGTCGCCCCTGGTGGACAACATCGCCCTGTGGATGACTGAG |
| | | TACCTGAACCGGCACCTGCACACCTGGATCCAGGATAACGGAGGCTGGGTAGGT |
| | | GCACTTGGTGATGTGAGTCTGGGCTGAGGCCACAGGTCCGAGATGCGGGGGTTG |
| | | GAGTGCGGGTGGGCTCCTGGGGCAATGGGAGGCTGTGGAGCCGGCGAAATAAA |
| | | ATCAGAGTTGTTGCT |
| SEQ | miR- | TGTCGGGTAGCTTATCAGACTGATGTTGACTGTTGAATCTCATGGCAACACCAGT |
| ID | 21 | CGATGGGCTGTCTGACA |
| NO: | | |
| 411 | | |
| SEQ | CCR5 | CTTCAGATAGATTATATCTGGAGTGAAGAATCCTGCCACCTATGTATCTGGCATA |
| ID | | GTATTCTGTGTAGTGGGATGAGCAGAGAACAAAAACAAAATAATCCAGTGAGAA |
| NO: | | AAGCCCGTAAATAAACCTTCAGACCAGAGATCTATTCTCTAGCTTATTTTAAGCT |
| 412 | | CAACTTAAAAAGAAGAACTGTTCTCTGATTCTTTTCGCCTTCAATACACTTAATG |
| | | ATTTAACTCCACCCTCCTTCAAAAGAAACAGCATTTCCTACTTTTATACTGTCTAT |
| | | ATGATTGATTTGCACAGCTCATCTGGCCAGAAGAGCTGAGACATCCGTTCCCCTA |
| | | CAAGAAACTCTCCCCGGGTGGAACAAGATGGATTATCAAGTGTCAAGTCCAATC |
| | | TATGACATCAATTATTATACATCGGAGCCCTGCCAAAAAATCAATGTGAAGCAA |
| | | ATCGCAGCCCGCCTCCTGCCTCCGCTCTACTCACTGGTGTTCATCTTTGGTTTTGT |
| | | GGGCAACATGCTGGTCATCCTCATCCTGATAAACTGCAAAAGGCTGAAGAGCAT |
| | | GACTGACATCTACCTGCTCAACCTGGCCATCTCTGACCTGTTTTTCCTTCTTACTG |
| | | TCCCCTTCTGGGCTCACTATGCTGCCGCCCAGTGGGACTTTGGAAATACAATGTG |
| | | TCAACTCTTGACAGGGCTCTATTTTATAGGCTTCTTCTCTGGAATCTTCTTCATCA |
| | | TCCTCCTGACAATCGATAGGTACCTGGCTGTCGTCCATGCTGTGTTTGCTTTAAAA |
| | | GCCAGGACGGTCACCTTTGGGGTGGTGACAAGTGTGATCACTTGGGTGGTGGCT |
| | | GTGTTTGCGTCTCTCCCAGGAATCATCTTTACCAGATCTCAAAAAGAAGGTCTTC |
| | | ATTACACCTGCAGCTCTCATTTTCCATACAGTCAGTATCAATTCTGGAAGAATTTC |
| | | CAGACATTAAAGATAGTCATCTTGGGGCTGGTCCTGCCGCTGCTTGTCATGGTCA |
| | | TCTGCTACTCGGGAATCCTAAAAACTCTGCTTCGGTGTCGAAATGAGAAGAAGA |
| | | GGCACAGGGCTGTGAGGCTTATCTTCACCATCATGATTGTTTATTTTCTCTTCTGG |
| | | GCTCCCTACAACATTGTCCTTCTCCTGAACACCTTCCAGGAATTCTTTGGCCTGAA |
| | | TAATTGCAGTAGCTCTAACAGGTTGGACCAAGCTATGCAGGTGACAGAGACTCTT |
| | | GGGATGACGCACTGCTGCATCAACCCCATCATCTATGCCTTTGTCGGGGAGAAGT |
| | | TCAGAAACTACCTCTTAGTCTTCTTCCAAAAGCACATTGCCAAACGCTTCTGCAA |
| | | ATGCTGTTCTATTTTCCAGCAAGAGGCTCCCGAGCGAGCAAGCTCAGTTTACACC |
| | | CGATCCACTGGGGAGCAGGAAATATCTGTGGGCTTGTGACACGGACTCAAGTGG |
| | | GCTGGTGACCCAGTCAGAGTTGTGCACATGGCTTAGTTTTCATACACAGCCTGGG |
| | | CTGGGGGTGGGGTGGGAGAGGTCTTTTTTAAAAGGAAGTTACTGTTATAGAGGG |
| | | TCTAAGATTCATCCATTTATTTGGCATCTGTTTAAAGTAGATTAGATCTTTTAAGC |
| | | CCATCAATTATAGAAAGCCAAATCAAAATATGTTGATGAAAAATAGCAACCTTTT |
| | | TATCTCCCCTTCACATGCATCAAGTTATTGACAAACTCTCCCTTCACTCCGAAAGT |
| | | TCCTTATGTATATTTAAAAGAAAGCCTCAGAGAATTGCTGATTCTTGAGTTTAGT |
| | | GATCTGAACAGAAATACCAAAATTATTTCAGAAATGTACAACTTTTTACCTAGTA |
| | | CAAGGCAACATATAGGTTGTAAATGTGTTTAAAACAGGTCTTTGTCTTGCTATGG |
| | | GGAGAAAAGACATGAATATGATTAGTAAAGAAATGACACTTTTCATGTGTGATT |
| | | TCCCCTCCAAGGTATGGTTAATAAGTTTCACTGACTTAGAACCAGGCGAGAGACT |
| | | TGTGGCCTGGGAGAGCTGGGGAAGCTTCTTAAATGAGAAGGAATTTGAGTTGGA |
| | | TCATCTATTGCTGGCAAAGACAGAAGCCTCACTGCAAGCACTGCATGGGCAAGC |
| | | TTGGCTGTAGAAGGAGACAGAGCTGGTTGGGAAGACATGGGGAGGAAGGACAA |
| | | GGCTAGATCATGAAGAACCTTGACGGCATTGCTCCGTCTAAGTCATGAGCTGAGC |
| | | AGGGAGATCCTGGTTGGTGTTGCAGAAGGTTTACTCTGTGGCCAAAGGAGGGTC |
| | | AGGAAGGATGAGCATTTAGGGCAAGGAGACCACCAACAGCCCTCAGGTCAGGGT |
| | | GAGGATGGCCTCTGCTAAGCTCAAGGCGTGAGGATGGGAAGGAGGGAGGTATTC |
| | | GTAAGGATGGGAAGGAGGGAGGTATTCGTGCAGCATATGAGGATGCAGAGTCA |
| | | GCAGAACTGGGGTGGATTTGGGTTGGAAGTGAGGGTCAGAGAGGAGTCAGAGA |
| | | GAATCCCTAGTCTTCAAGCAGATTGGAGAAACCCTTGAAAAGACATCAAGCACA |
| | | GAAGGAGGAGGAGGAGGTTTAGGTCAAGAAGAAGATGGATTGGTGTAAAAGGA |
| | | TGGGTCTGGTTTGCAGAGCTTGAACACAGTCTCACCCAGACTCCAGGCTGTCTTT |
| | | CACTGAATGCTTCTGACTTCATAGATTTCCTTCCCATCCCAGCTGAAATACTGAG |
| | | GGGTCTCCAGGAGGAGACTAGATTTATGAATACACGAGGTATGAGGTCTAGGAA |
| | | CATACTTCAGCTCACACATGAGATCTAGGTGAGGATTGATTACCTAGTAGTCATT |
| | | TCATGGGTTGTTGGGAGGATTCTATGAGGCAACCACAGGCAGCATTTAGCACAT |
| | | ACTACACATTCAATAAGCATCAAACTCTTAGTTACTCATTCAGGGATAGCACTGA |
| | | GCAAAGCATTGAGCAAAGGGGTCCCATAGAGGTGAGGGAAGCCTGAAAAACTA |
| | | AGATGCTGCCTGCCCAGTGCACACAAGTGTAGGTATCATTTTCTGCATTTAACCG |
| | | TCAATAGGCAAAGGGGGGAAGGGACATATTCATTTGGAAATAAGCTGCCTTGAG |
| | | CCTTAAAACCCACAAAAGTACAATTTACCAGCCTCCGTATTTCAGACTGAATGGG |
| | | GGTGGGGGGGGCGCCTTAGGTACTTATTCCAGATGCCTTCTCCAGACAAACCAG |
| | | AAGCAACAGAAAAAATCGTCTCTCCCTCCCTTTGAAATGAATATACCCCTTAGTG |
| | | TTTGGGTATATTCATTTCAAAGGGAGAGAGAGAGGTTTTTTTCTGTTCTGTCTCAT |
| | | ATGATTGTGCACATACTTGAGACTGTTTTGAATTTGGGGGATGGCTAAAACCATC |
| | | ATAGTACAGGTAAGGTGAGGGAATAGTAAGTGGTGAGAACTACTCAGGGAATGA |
| | | AGGTGTCAGAATAATAAGAGGTGCTACTGACTTTCTCAGCCTCTGAATATGAACG |
| | | GTGAGCATTGTGGCTGTCAGCAGGAAGCAACGAAGGGAAATGTCTTTCCTTTTGC |
| | | TCTTAAGTTGTGGAGAGTGCAACAGTAGCATAGGACCCTACCCTCTGGGCCAAGT |
| | | CAAAGACATTCTGACATCTTAGTATTTGCATATTCTTATGTATGTGAAAGTTACA |
| | | AATTGCTTGAAAGAAAATATGCATCTAATAAAAAACACCTTCTAAAATAA |
| SEQ | SOD1 | GCGTCGTAGTCTCCTGCAGCGTCTGGGGTTTCCGTTGCAGTCCTCGGAACCAGGA |
| ID | | CCTCGGCGTGGCCTAGCGAGTTATGGCGACGAAGGCCGTGTGCGTGCTGAAGGG |
| NO: | | CGACGGCCCAGTGCAGGGCATCATCAATTTCGAGCAGAAGGAAAGTAATGGACC |
| 413 | | AGTGAAGGTGTGGGGAAGCATTAAAGGACTGACTGAAGGCCTGCATGGATTCCA |
| | | TGTTCATGAGTTTGGAGATAATACAGCAGGCTGTACCAGTGCAGGTCCTCACTTT |
| | | AATCCTCTATCCAGAAAACACGGTGGGCCAAAGGATGAAGAGAGGCATGTTGGA |
| | | GACTTGGGCAATGTGACTGCTGACAAAGATGGTGTGGCCGATGTGTCTATTGAA |
| | | GATTCTGTGATCTCACTCTCAGGAGACCATTGCATCATTGGCCGCACACTGGTGG |
| | | TCCATGAAAAAGCAGATGACTTGGGCAAAGGTGGAAATGAAGAAAGTACAAAG |
| | | ACAGGAAACGCTGGAAGTCGTTTGGCTTGTGGTGTAATTGGGATCGCCCAATAA |
| | | ACATTCCCTTGGATGTAGTCTGAGGCCCCTTAACTCATCTGTTATCCTGCTAGCTG |
| | | TAGAAATGTATCCTGATAAACATTAAACACTGTAATCTTAAAAGTGTAATTGTGT |
| | | GACTTTTTCAGAGTTGCTTTAAAGTACCTGTAGTGAGAAACTGATTTATGATCAC |
| | | TTGGAAGATTTGTATAGTTTTATAAAACTCAGTTAAAATGTCTGTTTCAATGACCT |
| | | GTATTTTGCCAGACTTAAATCACAGATGGGTATTAAACTTGTCAGAATTTCTTTGT |
| | | CATTCAAGCCTGTGAATAAAAACCCTGTATGGCACTTATTATGAGGCTATTAAAA |
| | | GAATCCAAATTCAAACTAAA |
| SEQ | EGFR | AGACGTCCGGGCAGCCCCCGGCGCAGCGCGGCCGCAGCAGCCTCCGCCCCCCGC |
| ID | | ACGGTGTGAGCGCCCGACGCGGCCGAGGCGGCCGGAGTCCCGAGCTAGCCCCGG |
| NO: | | CGGCCGCCGCCGCCCAGACCGGACGACAGGCCACCTCGTCGGCGTCCGCCCGAG |
| 414 | | TCCCCGCCTCGCCGCCAACGCCACAACCACCGCGCACGGCCCCCTGACTCCGTCC |
| | | AGTATTGATCGGGAGAGCCGGAGCGAGCTCTTCGGGGAGCAGCGATGCGACCCT |
| | | CCGGGACGGCCGGGGCAGCGCTCCTGGCGCTGCTGGCTGCGCTCTGCCCGGCGA |
| | | GTCGGGCTCTGGAGGAAAAGAAAGTTTGCCAAGGCACGAGTAACAAGCTCACGC |
| | | AGTTGGGCACTTTTGAAGATCATTTTCTCAGCCTCCAGAGGATGTTCAATAACTG |
| | | TGAGGTGGTCCTTGGGAATTTGGAAATTACCTATGTGCAGAGGAATTATGATCTT |
| | | TCCTTCTTAAAGACCATCCAGGAGGTGGCTGGTTATGTCCTCATTGCCCTCAACA |
| | | CAGTGGAGCGAATTCCTTTGGAAAACCTGCAGATCATCAGAGGAAATATGTACT |
| | | ACGAAAATTCCTATGCCTTAGCAGTCTTATCTAACTATGATGCAAATAAAACCGG |
| | | ACTGAAGGAGCTGCCCATGAGAAATTTACAGGAAATCCTGCATGGCGCCGTGCG |
| | | GTTCAGCAACAACCCTGCCCTGTGCAACGTGGAGAGCATCCAGTGGCGGGACAT |
| | | AGTCAGCAGTGACTTTCTCAGCAACATGTCGATGGACTTCCAGAACCACCTGGGC |
| | | AGCTGCCAAAAGTGTGATCCAAGCTGTCCCAATGGGAGCTGCTGGGGTGCAGGA |
| | | GAGGAGAACTGCCAGAAACTGACCAAAATCATCTGTGCCCAGCAGTGCTCCGGG |
| | | CGCTGCCGTGGCAAGTCCCCCAGTGACTGCTGCCACAACCAGTGTGCTGCAGGCT |
| | | GCACAGGCCCCCGGGAGAGCGACTGCCTGGTCTGCCGCAAATTCCGAGACGAAG |
| | | CCACGTGCAAGGACACCTGCCCCCCACTCATGCTCTACAACCCCACCACGTACCA |
| | | GATGGATGTGAACCCCGAGGGCAAATACAGCTTTGGTGCCACCTGCGTGAAGAA |
| | | GTGTCCCCGTAATTATGTGGTGACAGATCACGGCTCGTGCGTCCGAGCCTGTGGG |
| | | GCCGACAGCTATGAGATGGAGGAAGACGGCGTCCGCAAGTGTAAGAAGTGCGA |
| | | AGGGCCTTGCCGCAAAGTGTGTAACGGAATAGGTATTGGTGAATTTAAAGACTC |
| | | ACTCTCCATAAATGCTACGAATATTAAACACTTCAAAAACTGCACCTCCATCAGT |
| | | GGCGATCTCCACATCCTGCCGGTGGCATTTAGGGGTGACTCCTTCACACATACTC |
| | | CTCCTCTGGATCCACAGGAACTGGATATTCTGAAAACCGTAAAGGAAATCACAG |
| | | GGTTTTTGCTGATTCAGGCTTGGCCTGAAAACAGGACGGACCTCCATGCCTTTGA |
| | | GAACCTAGAAATCATACGCGGCAGGACCAAGCAACATGGTCAGTTTTCTCTTGC |
| | | AGTCGTCAGCCTGAACATAACATCCTTGGGATTACGCTCCCTCAAGGAGATAAGT |
| | | GATGGAGATGTGATAATTTCAGGAAACAAAAATTTGTGCTATGCAAATACAATA |
| | | AACTGGAAAAAACTGTTTGGGACCTCCGGTCAGAAAACCAAAATTATAAGCAAC |
| | | AGAGGTGAAAACAGCTGCAAGGCCACAGGCCAGGTCTGCCATGCCTTGTGCTCC |
| | | CCCGAGGGCTGCTGGGGCCCGGAGCCCAGGGACTGCGTCTCTTGCCGGAATGTC |
| | | AGCCGAGGCAGGGAATGCGTGGACAAGTGCAACCTTCTGGAGGGTGAGCCAAG |
| | | GGAGTTTGTGGAGAACTCTGAGTGCATACAGTGCCACCCAGAGTGCCTGCCTCA |
| | | GGCCATGAACATCACCTGCACAGGACGGGGACCAGACAACTGTATCCAGTGTGC |
| | | CCACTACATTGACGGCCCCCACTGCGTCAAGACCTGCCCGGCAGGAGTCATGGG |
| | | AGAAAACAACACCCTGGTCTGGAAGTACGCAGACGCCGGCCATGTGTGCCACCT |
| | | GTGCCATCCAAACTGCACCTACGGATGCACTGGGCCAGGTCTTGAAGGCTGTCCA |
| | | ACGAATGGGCCTAAGATCCCGTCCATCGCCACTGGGATGGTGGGGGCCCTCCTCT |
| | | TGCTGCTGGTGGTGGCCCTGGGGATCGGCCTCTTCATGCGAAGGCGCCACATCGT |
| | | TCGGAAGCGCACGCTGCGGAGGCTGCTGCAGGAGAGGGAGCTTGTGGAGCCTCT |
| | | TACACCCAGTGGAGAAGCTCCCAACCAAGCTCTCTTGAGGATCTTGAAGGAAAC |
| | | TGAATTCAAAAAGATCAAAGTGCTGGGCTCCGGTGCGTTCGGCACGGTGTATAA |
| | | GGGACTCTGGATCCCAGAAGGTGAGAAAGTTAAAATTCCCGTCGCTATCAAGGA |
| | | ATTAAGAGAAGCAACATCTCCGAAAGCCAACAAGGAAATCCTCGATGAAGCCTA |
| | | CGTGATGGCCAGCGTGGACAACCCCCACGTGTGCCGCCTGCTGGGCATCTGCCTC |
| | | ACCTCCACCGTGCAGCTCATCACGCAGCTCATGCCCTTCGGCTGCCTCCTGGACT |
| | | ATGTCCGGGAACACAAAGACAATATTGGCTCCCAGTACCTGCTCAACTGGTGTGT |
| | | GCAGATCGCAAAGGGCATGAACTACTTGGAGGACCGTCGCTTGGTGCACCGCGA |
| | | CCTGGCAGCCAGGAACGTACTGGTGAAAACACCGCAGCATGTCAAGATCACAGA |
| | | TTTTGGGCTGGCCAAACTGCTGGGTGCGGAAGAGAAAGAATACCATGCAGAAGG |
| | | AGGCAAAGTGCCTATCAAGTGGATGGCATTGGAATCAATTTTACACAGAATCTAT |
| | | ACCCACCAGAGTGATGTCTGGAGCTACGGGGTGACTGTTTGGGAGTTGATGACCT |
| | | TTGGATCCAAGCCATATGACGGAATCCCTGCCAGCGAGATCTCCTCCATCCTGGA |
| | | GAAAGGAGAACGCCTCCCTCAGCCACCCATATGTACCATCGATGTCTACATGATC |
| | | ATGGTCAAGTGCTGGATGATAGACGCAGATAGTCGCCCAAAGTTCCGTGAGTTG |
| | | ATCATCGAATTCTCCAAAATGGCCCGAGACCCCCAGCGCTACCTTGTCATTCAGG |
| | | GGGATGAAAGAATGCATTTGCCAAGTCCTACAGACTCCAACTTCTACCGTGCCCT |
| | | GATGGATGAAGAAGACATGGACGACGTGGTGGATGCCGACGAGTACCTCATCCC |
| | | ACAGCAGGGCTTCTTCAGCAGCCCCTCCACGTCACGGACTCCCCTCCTGAGCTCT |
| | | CTGAGTGCAACCAGCAACAATTCCACCGTGGCTTGCATTGATAGAAATGGGCTG |
| | | CAAAGCTGTCCCATCAAGGAAGACAGCTTCTTGCAGCGATACAGCTCAGACCCC |
| | | ACAGGCGCCTTGACTGAGGACAGCATAGACGACACCTTCCTCCCAGTGCCTGAA |
| | | TACATAAACCAGTCCGTTCCCAAAAGGCCCGCTGGCTCTGTGCAGAATCCTGTCT |
| | | ATCACAATCAGCCTCTGAACCCCGCGCCCAGCAGAGACCCACACTACCAGGACC |
| | | CCCACAGCACTGCAGTGGGCAACCCCGAGTATCTCAACACTGTCCAGCCCACCTG |
| | | TGTCAACAGCACATTCGACAGCCCTGCCCACTGGGCCCAGAAAGGCAGCCACCA |
| | | AATTAGCCTGGACAACCCTGACTACCAGCAGGACTTCTTTCCCAAGGAAGCCAA |
| | | GCCAAATGGCATCTTTAAGGGCTCCACAGCTGAAAATGCAGAATACCTAAGGGT |
| | | CGCGCCACAAAGCAGTGAATTTATTGGAGCATGACCACGGAGGATAGTATGAGC |
| | | CCTAAAAATCCAGACTCTTTCGATACCCAGGACCAAGCCACAGCAGGTCCTCCAT |
| | | CCCAACAGCCATGCCCGCATTAGCTCTTAGACCCACAGACTGGTTTTGCAACGTT |
| | | TACACCGACTAGCCAGGAAGTACTTCCACCTCGGGCACATTTTGGGAAGTTGCAT |
| | | TCCTTTGTCTTCAAACTGTGAAGCATTTACAGAAACGCATCCAGCAAGAATATTG |
| | | TCCCTTTGAGCAGAAATTTATCTTTCAAAGAGGTATATTTGAAAAAAAAAAAAA |
| | | GTATATGTGAGGATTTTTATTGATTGGGGATCTTGGAGTTTTTCATTGTCGCTATT |
| | | GATTTTTACTTCAATGGGCTCTTCCAACAAGGAAGAAGCTTGCTGGTAGCACTTG |
| | | CTACCCTGAGTTCATCCAGGCCCAACTGTGAGCAAGGAGCACAAGCCACAAGTC |
| | | TTCCAGAGGATGCTTGATTCCAGTGGTTCTGCTTCAAGGCTTCCACTGCAAAACA |
| | | CTAAAGATCCAAGAAGGCCTTCATGGCCCCAGCAGGCCGGATCGGTACTGTATC |
| | | AAGTCATGGCAGGTACAGTAGGATAAGCCACTCTGTCCCTTCCTGGGCAAAGAA |
| | | GAAACGGAGGGGATGGAATTCTTCCTTAGACTTACTTTTGTAAAAATGTCCCCAC |
| | | GGTACTTACTCCCCACTGATGGACCAGTGGTTTCCAGTCATGAGCGTTAGACTGA |
| | | CTTGTTTGTCTTCCATTCCATTGTTTTGAAACTCAGTATGCTGCCCCTGTCTTGCTG |
| | | TCATGAAATCAGCAAGAGAGGATGACACATCAAATAATAACTCGGATTCCAGCC |
| | | CACATTGGATTCATCAGCATTTGGACCAATAGCCCACAGCTGAGAATGTGGAAT |
| | | ACCTAAGGATAGCACCGCTTTTGTTCTCGCAAAAACGTATCTCCTAATTTGAGGC |
| | | TCAGATGAAATGCATCAGGTCCTTTGGGGCATAGATCAGAAGACTACAAAAATG |
| | | AAGCTGCTCTGAAATCTCCTTTAGCCATCACCCCAACCCCCCAAAATTAGTTTGT |
| | | GTTACTTATGGAAGATAGTTTTCTCCTTTTACTTCACTTCAAAAGCTTTTTACTCA |
| | | AAGAGTATATGTTCCCTCCAGGTCAGCTGCCCCCAAACCCCCTCCTTACGCTTTG |
| | | TCACACAAAAAGTGTCTCTGCCTTGAGTCATCTATTCAAGCACTTACAGCTCTGG |
| | | CCACAACAGGGCATTTTACAGGTGCGAATGACAGTAGCATTATGAGTAGTGTGG |
| | | AATTCAGGTAGTAAATATGAAACTAGGGTTTGAAATTGATAATGCTTTCACAACA |
| | | TTTGCAGATGTTTTAGAAGGAAAAAAGTTCCTTCCTAAAATAATTTCTCTACAAT |
| | | TGGAAGATTGGAAGATTCAGCTAGTTAGGAGCCCACCTTTTTTCCTAATCTGTGT |
| | | GTGCCCTGTAACCTGACTGGTTAACAGCAGTCCTTTGTAAACAGTGTTTTAAACT |
| | | CTCCTAGTCAATATCCACCCCATCCAATTTATCAAGGAAGAAATGGTTCAGAAAA |
| | | TATTTTCAGCCTACAGTTATGTTCAGTCACACACACATACAAAATGTTCCTTTTGC |
| | | TTTTAAAGTAATTTTTGACTCCCAGATCAGTCAGAGCCCCTACAGCATTGTTAAG |
| | | AAAGTATTTGATTTTTGTCTCAATGAAAATAAAACTATATTCATTTCCACTCTATT |
| | | ATGCTCTCAAATACCCCTAAGCATCTATACTAGCCTGGTATGGGTATGAAAGATA |
| | | CAAAGATAAATAAAACATAGTCCCTGATTCTAAGAAATTCACAATTTAGCAAAG |
| | | GAAATGGACTCATAGATGCTAACCTTAAAACAACGTGACAAATGCCAGACAGGA |
| | | CCCATCAGCCAGGCACTGTGAGAGCACAGAGCAGGGAGGTTGGGTCCTGCCTGA |
| | | GGAGACCTGGAAGGGAGGCCTCACAGGAGGATGACCAGGTCTCAGTCAGCGGG |
| | | GAGGTGGAAAGTGCAGGTGCATCAGGGGCACCCTGACCGAGGAAACAGCTGCC |
| | | AGAGGCCTCCACTGCTAAAGTCCACATAAGGCTGAGGTCAGTCACCCTAAACAA |
| | | CCTGCTCCCTCTAAGCCAGGGGATGAGCTTGGAGCATCCCACAAGTTCCCTAAAA |
| | | GTTGCAGCCCCCAGGGGGATTTTGAGCTATCATCTCTGCACATGCTTAGTGAGAA |
| | | GACTACACAACATTTCTAAGAATCTGAGATTTTATATTGTCAGTTAACCACTTTC |
| | | ATTATTCATTCACCTCAGGACATGCAGAAATATTTCAGTCAGAACTGGGAAACAG |
| | | AAGGACCTACATTCTGCTGTCACTTATGTGTCAAGAAGCAGATGATCGATGAGGC |
| | | AGGTCAGTTGTAAGTGAGTCACATTGTAGCATTAAATTCTAGTATTTTTGTAGTTT |
| | | GAAACAGTAACTTAATAAAAGAGCAAAAGCTATTCTAGCTTTCTTCTTCATATTT |
| | | TAATTTTCCACCATAAAGTTTAGTTGCTAAATTCTATTAATTTTAAGATTGTGCTT |
| | | CCCAAAATAGTTCTCACTTCATCTGTCCAGGGAGGCACAGTTCTGTCTGGTAGAA |
| | | GCCGCAAAGCCCTTAGCCTCTTCACGGATCTGGCGACTGTGATGGGCAGGTCAG |
| | | GAGAGGAGCTGCCCAAAGTCCCATGATTTTCACCTAACAGCCCTGATCAGTCAGT |
| | | ACTCAAAGCTTGGACTCCATCCCTGAAGGTCTTCCTGATTGATAGCCTGGCCTTA |
| | | ATACCCTACAGAAAGCCTGTCCATTGGCTGTTTCTTCCTCAGTCAGTTCCTGGAA |
| | | GACCTTACCCCATGACCCCAGCTTCAGATGTGGTCTTTGGAAACAGAGGTCGAAG |
| | | GAAAGTAAGGAGCTGAGAGCTCACATTCATAGGTGCCGCCAGCCTTCGTGCATC |
| | | TTCTTGCATCATCTCTAAGGAGCTCCTCTAATTACACCATGCCCGTCACCCCATGA |
| | | GGGATCAGAGAAGGGATGAGTCTTCTAAACTCTATATTCGCTGTGAGTCCAGGTT |
| | | GTAAGGGGGAGCACTGTGGATGCATCCTATTGCACTCCAGCTGATGACACCAAA |
| | | GCTTAGGTGTTTGCTGAAAGTTCTTGATGTTGTGACTTACCACCCCTGCCTCACAA |
| | | CTGCAGACATAAGGGGACTATGGATTGCTTAGCAGGAAAGGCACTGGTTCTCAA |
| | | GGGCGGCTGCCCTTGGGAATCTTCTGGTCCCAACCAGAAAGACTGTGGCTTGATT |
| | | TTCTCAGGTGCAGCCCAGCCGTAGGGCCTTTTCAGAGCACCCCCTGGTTATTGCA |
| | | ACATTCATCAAAGTTTCTAGAACCTCTGGCCTAAAGGAAGGGCCTGGTGGGATCT |
| | | ACTTGGCACTCGCTGGGGGGCCACCCCCCAGTGCCACTCTCACTAGGCCTCTGAT |
| | | TGCACTTGTGTAGGATGAAGCTGGTGGGTGATGGGAACTCAGCACCTCCCCTCAG |
| | | GCAGAAAAGAATCATCTGTGGAGCTTCAAAAGAAGGGGCCTGGAGTCTCTGCAG |
| | | ACCAATTCAACCCAAATCTCGGGGGCTCTTTCATGATTCTAATGGGCAACCAGGG |
| | | TTGAAACCCTTATTTCTAGGGTCTTCAGTTGTACAAGACTGTGGGTCTGTACCAG |
| | | AGCCCCCGTCAGAGTAGAATAAAAGGCTGGGTAGGGTAGAGATTCCCATGTGCA |
| | | GTGGAGAGAACAATCTGCAGTCACTGATAAGCCTGAGACTTGGCTCATTTCAAA |
| | | AGCGTTCAATTCATCCTCACCAGCAGTTCAGCTGGAAAGGGGCAAATACCCCCA |
| | | CCTGAGCTTTGAAAACGCCCTGGGACCCTCTGCATTCTCTAAGTAAGTTATAGAA |
| | | ACCAGTCTCTTCCCTCCTTTGTGAGTGAGCTGCTATTCCACGTAGGCAACACCTGT |
| | | TGAAATTGCCCTCAATGTCTACTCTGCATTTCTTTCTTGTGATAAGCACACACTTT |
| | | TATTGCAACATAATGATCTGCTCACATTTCCTTGCCTGGGGGCTGTAAAACCTTA |
| | | CAGAACAGAAATCCTTGCCTCTTTCACCAGCCACACCTGCCATACCAGGGGTACA |
| | | GCTTTGTACTATTGAAGACACAGACAGGATTTTTAAATGTAAATCTATTTTTGTA |
| | | ACTTTGTTGCGGGATATAGTTCTCTTTATGTAGCACTGAACTTTGTACAATATATT |
| | | TTTAGAAACTCATTTTTCTACTAAAACAAACACAGTTTACTTTAGAGAGACTGCA |
| | | ATAGAATCAAAATTTGAAACTGAAATCTTTGTTTAAAAGGGTTAAGTTGAGGCA |
| | | AGAGGAAAGCCCTTTCTCTCTCTTATAAAAAGGCACAACCTCATTGGGGAGCTAA |
| | | GCTAGGTCATTGTCATGGTGAAGAAGAGAAGCATCGTTTTTATATTTAGGAAATT |
| | | TTAAAAGATGATGGAAAGCACATTTAGCTTGGTCTGAGGCAGGTTCTGTTGGGGC |
| | | AGTGTTAATGGAAAGGGCTCACTGTTGTTACTACTAGAAAAATCCAGTTGCATGC |
| | | CATACTCTCATCATCTGCCAGTGTAACCCTGTACATGTAAGAAAAGCAATAACAT |
| | | AGCACTTTGTTGGTTTATATATATAATGTGACTTCAATGCAAATTTTATTTTTATA |
| | | TTTACAATTGATATGCATTTACCAGTATAAACTAGACATGTCTGGAGAGCCTAAT |
| | | AATGTTCAGCACACTTTGGTTAGTTCACCAACAGTCTTACCAAGCCTGGGCCCAG |
| | | CCACCCTAGAGAAGTTATTCAGCCCTGGCTGCAGTGACATCACCTGAGGAGCTTT |
| | | TAAAAGCTTGAAGCCCAGCTACACCTCAGACCGATTAAACGCAAATCTCTGGGG |
| | | CTGAAACCCAAGCATTCGTAGTTTTTAAAGCTCCTGAGGTCATTCCAATGTGCGG |
| | | CCAAAGTTGAGAACTACTGGCCTAGGGATTAGCCACAAGGACATGGACTTGGAG |
| | | GCAAATTCTGCAGGTGTATGTGATTCTCAGGCCTAGAGAGCTAAGACACAAAGA |
| | | CCTCCACATCTGTCGCTGAGAGTCAAGAACCTGAACAGAGTTTCCATGAAGGTTC |
| | | TCCAAGCACTAGAAGGGAGAGTGTCTAAACAATGGTTGAAAAGCAAAGGAAAT |
| | | ATAAAACAGACACCTCTTTCCATTTCCTAAGGTTTCTCTCTTTATTAAGGGTGGAC |
| | | TAGTAATAAAATATAATATTCTTGCTGCTTATGCAGCTGACATTGTTGCCCTCCCT |
| | | AAAGCAACCAAGTAGCCTTTATTTCCCACAGTGAAAGAAAACGCTGGCCTATCA |
| | | GTTACATTACAAAAGGCAGATTTCAAGAGGATTGAGTAAGTAGTTGGATGGCTTT |
| | | CATAAAAACAAGAATTCAAGAAGAGGATTCATGCTTTAAGAAACATTTGTTATA |
| | | CATTCCTCACAAATTATACCTGGGATAAAAACTATGTAGCAGGCAGTGTGTTTTC |
| | | CTTCCATGTCTCTCTGCACTACCTGCAGTGTGTCCTCTGAGGCTGCAAGTCTGTCC |
| | | TATCTGAATTCCCAGCAGAAGCACTAAGAAGCTCCACCCTATCACCTAGCAGATA |
| | | AAACTATGGGGAAAACTTAAATCTGTGCATACATTTCTGGATGCATTTACTTATC |
| | | TTTAAAAAAAAAGGAATCCTATGACCTGATTTGGCCACAAAAATAATCTTGCTGT |
| | | ACAATACAATCTCTTGGAAATTAAGAGATCCTATGGATTTGATGACTGGTATTAG |
| | | AGGTGACAATGTAACCGATTAACAACAGACAGCAATAACTTCGTTTTAGAAACA |
| | | TTCAAGCAATAGCTTTATAGCTTCAACATATGGTACGTTTTAACCTTGAAAGTTTT |
| | | GCAATGATGAAAGCAGTATTTGTACAAATGAAAAGCAGAATTCTCTTTTATATGG |
| | | TTTATACTGTTGATCAGAAATGTTGATTGTGCATTGAGTATTAAAAAATTAGATG |
| | | TATATTATTCATTGTTCTTTACTCCTGAGTACCTTATAATAATAATAATGTATTCT |
| | | TTGTTAACAA |
| SEQ | EGFR | AGACGTCCGGGCAGCCCCCGGCGCAGCGCGGCCGCAGCAGCCTCCGCCCCCCGC |
| ID | | ACGGTGTGAGCGCCCGACGCGGCCGAGGCGGCCGGAGTCCCGAGCTAGCCCCGG |
| NO: | | CGGCCGCCGCCGCCCAGACCGGACGACAGGCCACCTCGTCGGCGTCCGCCCGAG |
| 415 | | TCCCCGCCTCGCCGCCAACGCCACAACCACCGCGCACGGCCCCCTGACTCCGTCC |
| | | AGTATTGATCGGGAGAGCCGGAGCGAGCTCTTCGGGGAGCAGCGATGCGACCCT |
| | | CCGGGACGGCCGGGGCAGCGCTCCTGGCGCTGCTGGCTGCGCTCTGCCCGGCGA |
| | | GTCGGGCTCTGGAGGAAAAGAAAGTTTGCCAAGGCACGAGTAACAAGCTCACGC |
| | | AGTTGGGCACTTTTGAAGATCATTTTCTCAGCCTCCAGAGGATGTTCAATAACTG |
| | | TGAGGTGGTCCTTGGGAATTTGGAAATTACCTATGTGCAGAGGAATTATGATCTT |
| | | TCCTTCTTAAAGACCATCCAGGAGGTGGCTGGTTATGTCCTCATTGCCCTCAACA |
| | | CAGTGGAGCGAATTCCTTTGGAAAACCTGCAGATCATCAGAGGAAATATGTACT |
| | | ACGAAAATTCCTATGCCTTAGCAGTCTTATCTAACTATGATGCAAATAAAACCGG |
| | | ACTGAAGGAGCTGCCCATGAGAAATTTACAGGGCCAAAAGTGTGATCCAAGCTG |
| | | TCCCAATGGGAGCTGCTGGGGTGCAGGAGAGGAGAACTGCCAGAAACTGACCAA |
| | | AATCATCTGTGCCCAGCAGTGCTCCGGGCGCTGCCGTGGCAAGTCCCCCAGTGAC |
| | | TGCTGCCACAACCAGTGTGCTGCAGGCTGCACAGGCCCCCGGGAGAGCGACTGC |
| | | CTGGTCTGCCGCAAATTCCGAGACGAAGCCACGTGCAAGGACACCTGCCCCCCA |
| | | CTCATGCTCTACAACCCCACCACGTACCAGATGGATGTGAACCCCGAGGGCAAA |
| | | TACAGCTTTGGTGCCACCTGCGTGAAGAAGTGTCCCCGTAATTATGTGGTGACAG |
| | | ATCACGGCTCGTGCGTCCGAGCCTGTGGGGCCGACAGCTATGAGATGGAGGAAG |
| | | ACGGCGTCCGCAAGTGTAAGAAGTGCGAAGGGCCTTGCCGCAAAGTGTGTAACG |
| | | GAATAGGTATTGGTGAATTTAAAGACTCACTCTCCATAAATGCTACGAATATTAA |
| | | ACACTTCAAAAACTGCACCTCCATCAGTGGCGATCTCCACATCCTGCCGGTGGCA |
| | | TTTAGGGGTGACTCCTTCACACATACTCCTCCTCTGGATCCACAGGAACTGGATA |
| | | TTCTGAAAACCGTAAAGGAAATCACAGGGTTTTTGCTGATTCAGGCTTGGCCTGA |
| | | AAACAGGACGGACCTCCATGCCTTTGAGAACCTAGAAATCATACGCGGCAGGAC |
| | | CAAGCAACATGGTCAGTTTTCTCTTGCAGTCGTCAGCCTGAACATAACATCCTTG |
| | | GGATTACGCTCCCTCAAGGAGATAAGTGATGGAGATGTGATAATTTCAGGAAAC |
| | | AAAAATTTGTGCTATGCAAATACAATAAACTGGAAAAAACTGTTTGGGACCTCC |
| | | GGTCAGAAAACCAAAATTATAAGCAACAGAGGTGAAAACAGCTGCAAGGCCAC |
| | | AGGCCAGGTCTGCCATGCCTTGTGCTCCCCCGAGGGCTGCTGGGGCCCGGAGCCC |
| | | AGGGACTGCGTCTCTTGCCGGAATGTCAGCCGAGGCAGGGAATGCGTGGACAAG |
| | | TGCAACCTTCTGGAGGGTGAGCCAAGGGAGTTTGTGGAGAACTCTGAGTGCATA |
| | | CAGTGCCACCCAGAGTGCCTGCCTCAGGCCATGAACATCACCTGCACAGGACGG |
| | | GGACCAGACAACTGTATCCAGTGTGCCCACTACATTGACGGCCCCCACTGCGTCA |
| | | AGACCTGCCCGGCAGGAGTCATGGGAGAAAACAACACCCTGGTCTGGAAGTACG |
| | | CAGACGCCGGCCATGTGTGCCACCTGTGCCATCCAAACTGCACCTACGGATGCAC |
| | | TGGGCCAGGTCTTGAAGGCTGTCCAACGAATGGGCCTAAGATCCCGTCCATCGCC |
| | | ACTGGGATGGTGGGGGCCCTCCTCTTGCTGCTGGTGGTGGCCCTGGGGATCGGCC |
| | | TCTTCATGCGAAGGCGCCACATCGTTCGGAAGCGCACGCTGCGGAGGCTGCTGC |
| | | AGGAGAGGGAGCTTGTGGAGCCTCTTACACCCAGTGGAGAAGCTCCCAACCAAG |
| | | CTCTCTTGAGGATCTTGAAGGAAACTGAATTCAAAAAGATCAAAGTGCTGGGCT |
| | | CCGGTGCGTTCGGCACGGTGTATAAGGGACTCTGGATCCCAGAAGGTGAGAAAG |
| | | TTAAAATTCCCGTCGCTATCAAGGAATTAAGAGAAGCAACATCTCCGAAAGCCA |
| | | ACAAGGAAATCCTCGATGAAGCCTACGTGATGGCCAGCGTGGACAACCCCCACG |
| | | TGTGCCGCCTGCTGGGCATCTGCCTCACCTCCACCGTGCAGCTCATCACGCAGCT |
| | | CATGCCCTTCGGCTGCCTCCTGGACTATGTCCGGGAACACAAAGACAATATTGGC |
| | | TCCCAGTACCTGCTCAACTGGTGTGTGCAGATCGCAAAGGGCATGAACTACTTGG |
| | | AGGACCGTCGCTTGGTGCACCGCGACCTGGCAGCCAGGAACGTACTGGTGAAAA |
| | | CACCGCAGCATGTCAAGATCACAGATTTTGGGCTGGCCAAACTGCTGGGTGCGG |
| | | AAGAGAAAGAATACCATGCAGAAGGAGGCAAAGTGCCTATCAAGTGGATGGCA |
| | | TTGGAATCAATTTTACACAGAATCTATACCCACCAGAGTGATGTCTGGAGCTACG |
| | | GGGTGACTGTTTGGGAGTTGATGACCTTTGGATCCAAGCCATATGACGGAATCCC |
| | | TGCCAGCGAGATCTCCTCCATCCTGGAGAAAGGAGAACGCCTCCCTCAGCCACC |
| | | CATATGTACCATCGATGTCTACATGATCATGGTCAAGTGCTGGATGATAGACGCA |
| | | GATAGTCGCCCAAAGTTCCGTGAGTTGATCATCGAATTCTCCAAAATGGCCCGAG |
| | | ACCCCCAGCGCTACCTTGTCATTCAGGGGGATGAAAGAATGCATTTGCCAAGTCC |
| | | TACAGACTCCAACTTCTACCGTGCCCTGATGGATGAAGAAGACATGGACGACGT |
| | | GGTGGATGCCGACGAGTACCTCATCCCACAGCAGGGCTTCTTCAGCAGCCCCTCC |
| | | ACGTCACGGACTCCCCTCCTGAGCTCTCTGAGTGCAACCAGCAACAATTCCACCG |
| | | TGGCTTGCATTGATAGAAATGGGCTGCAAAGCTGTCCCATCAAGGAAGACAGCT |
| | | TCTTGCAGCGATACAGCTCAGACCCCACAGGCGCCTTGACTGAGGACAGCATAG |
| | | ACGACACCTTCCTCCCAGTGCCTGAATACATAAACCAGTCCGTTCCCAAAAGGCC |
| | | CGCTGGCTCTGTGCAGAATCCTGTCTATCACAATCAGCCTCTGAACCCCGCGCCC |
| | | AGCAGAGACCCACACTACCAGGACCCCCACAGCACTGCAGTGGGCAACCCCGAG |
| | | TATCTCAACACTGTCCAGCCCACCTGTGTCAACAGCACATTCGACAGCCCTGCCC |
| | | ACTGGGCCCAGAAAGGCAGCCACCAAATTAGCCTGGACAACCCTGACTACCAGC |
| | | AGGACTTCTTTCCCAAGGAAGCCAAGCCAAATGGCATCTTTAAGGGCTCCACAG |
| | | CTGAAAATGCAGAATACCTAAGGGTCGCGCCACAAAGCAGTGAATTTATTGGAG |
| | | CATGACCACGGAGGATAGTATGAGCCCTAAAAATCCAGACTCTTTCGATACCCA |
| | | GGACCAAGCCACAGCAGGTCCTCCATCCCAACAGCCATGCCCGCATTAGCTCTTA |
| | | GACCCACAGACTGGTTTTGCAACGTTTACACCGACTAGCCAGGAAGTACTTCCAC |
| | | CTCGGGCACATTTTGGGAAGTTGCATTCCTTTGTCTTCAAACTGTGAAGCATTTAC |
| | | AGAAACGCATCCAGCAAGAATATTGTCCCTTTGAGCAGAAATTTATCTTTCAAAG |
| | | AGGTATATTTGAAAAAAAAAAAAAGTATATGTGAGGATTTTTATTGATTGGGGA |
| | | TCTTGGAGTTTTTCATTGTCGCTATTGATTTTTACTTCAATGGGCTCTTCCAACAA |
| | | GGAAGAAGCTTGCTGGTAGCACTTGCTACCCTGAGTTCATCCAGGCCCAACTGTG |
| | | AGCAAGGAGCACAAGCCACAAGTCTTCCAGAGGATGCTTGATTCCAGTGGTTCT |
| | | GCTTCAAGGCTTCCACTGCAAAACACTAAAGATCCAAGAAGGCCTTCATGGCCC |
| | | CAGCAGGCCGGATCGGTACTGTATCAAGTCATGGCAGGTACAGTAGGATAAGCC |
| | | ACTCTGTCCCTTCCTGGGCAAAGAAGAAACGGAGGGGATGGAATTCTTCCTTAG |
| | | ACTTACTTTTGTAAAAATGTCCCCACGGTACTTACTCCCCACTGATGGACCAGTG |
| | | GTTTCCAGTCATGAGCGTTAGACTGACTTGTTTGTCTTCCATTCCATTGTTTTGAA |
| | | ACTCAGTATGCTGCCCCTGTCTTGCTGTCATGAAATCAGCAAGAGAGGATGACAC |
| | | ATCAAATAATAACTCGGATTCCAGCCCACATTGGATTCATCAGCATTTGGACCAA |
| | | TAGCCCACAGCTGAGAATGTGGAATACCTAAGGATAGCACCGCTTTTGTTCTCGC |
| | | AAAAACGTATCTCCTAATTTGAGGCTCAGATGAAATGCATCAGGTCCTTTGGGGC |
| | | ATAGATCAGAAGACTACAAAAATGAAGCTGCTCTGAAATCTCCTTTAGCCATCAC |
| | | CCCAACCCCCCAAAATTAGTTTGTGTTACTTATGGAAGATAGTTTTCTCCTTTTAC |
| | | TTCACTTCAAAAGCTTTTTACTCAAAGAGTATATGTTCCCTCCAGGTCAGCTGCCC |
| | | CCAAACCCCCTCCTTACGCTTTGTCACACAAAAAGTGTCTCTGCCTTGAGTCATCT |
| | | ATTCAAGCACTTACAGCTCTGGCCACAACAGGGCATTTTACAGGTGCGAATGAC |
| | | AGTAGCATTATGAGTAGTGTGGAATTCAGGTAGTAAATATGAAACTAGGGTTTG |
| | | AAATTGATAATGCTTTCACAACATTTGCAGATGTTTTAGAAGGAAAAAAGTTCCT |
| | | TCCTAAAATAATTTCTCTACAATTGGAAGATTGGAAGATTCAGCTAGTTAGGAGC |
| | | CCACCTTTTTTCCTAATCTGTGTGTGCCCTGTAACCTGACTGGTTAACAGCAGTCC |
| | | TTTGTAAACAGTGTTTTAAACTCTCCTAGTCAATATCCACCCCATCCAATTTATCA |
| | | AGGAAGAAATGGTTCAGAAAATATTTTCAGCCTACAGTTATGTTCAGTCACACAC |
| | | ACATACAAAATGTTCCTTTTGCTTTTAAAGTAATTTTTGACTCCCAGATCAGTCAG |
| | | AGCCCCTACAGCATTGTTAAGAAAGTATTTGATTTTTGTCTCAATGAAAATAAAA |
| | | CTATATTCATTTCCACTCTATTATGCTCTCAAATACCCCTAAGCATCTATACTAGC |
| | | CTGGTATGGGTATGAAAGATACAAAGATAAATAAAACATAGTCCCTGATTCTAA |
| | | GAAATTCACAATTTAGCAAAGGAAATGGACTCATAGATGCTAACCTTAAAACAA |
| | | CGTGACAAATGCCAGACAGGACCCATCAGCCAGGCACTGTGAGAGCACAGAGCA |
| | | GGGAGGTTGGGTCCTGCCTGAGGAGACCTGGAAGGGAGGCCTCACAGGAGGATG |
| | | ACCAGGTCTCAGTCAGCGGGGAGGTGGAAAGTGCAGGTGCATCAGGGGCACCCT |
| | | GACCGAGGAAACAGCTGCCAGAGGCCTCCACTGCTAAAGTCCACATAAGGCTGA |
| | | GGTCAGTCACCCTAAACAACCTGCTCCCTCTAAGCCAGGGGATGAGCTTGGAGC |
| | | ATCCCACAAGTTCCCTAAAAGTTGCAGCCCCCAGGGGGATTTTGAGCTATCATCT |
| | | CTGCACATGCTTAGTGAGAAGACTACACAACATTTCTAAGAATCTGAGATTTTAT |
| | | ATTGTCAGTTAACCACTTTCATTATTCATTCACCTCAGGACATGCAGAAATATTTC |
| | | AGTCAGAACTGGGAAACAGAAGGACCTACATTCTGCTGTCACTTATGTGTCAAG |
| | | AAGCAGATGATCGATGAGGCAGGTCAGTTGTAAGTGAGTCACATTGTAGCATTA |
| | | AATTCTAGTATTTTTGTAGTTTGAAACAGTAACTTAATAAAAGAGCAAAAGCTAT |
| | | TCTAGCTTTCTTCTTCATATTTTAATTTTCCACCATAAAGTTTAGTTGCTAAATTCT |
| | | ATTAATTTTAAGATTGTGCTTCCCAAAATAGTTCTCACTTCATCTGTCCAGGGAG |
| | | GCACAGTTCTGTCTGGTAGAAGCCGCAAAGCCCTTAGCCTCTTCACGGATCTGGC |
| | | GACTGTGATGGGCAGGTCAGGAGAGGAGCTGCCCAAAGTCCCATGATTTTCACC |
| | | TAACAGCCCTGATCAGTCAGTACTCAAAGCTTGGACTCCATCCCTGAAGGTCTTC |
| | | CTGATTGATAGCCTGGCCTTAATACCCTACAGAAAGCCTGTCCATTGGCTGTTTC |
| | | TTCCTCAGTCAGTTCCTGGAAGACCTTACCCCATGACCCCAGCTTCAGATGTGGT |
| | | CTTTGGAAACAGAGGTCGAAGGAAAGTAAGGAGCTGAGAGCTCACATTCATAGG |
| | | TGCCGCCAGCCTTCGTGCATCTTCTTGCATCATCTCTAAGGAGCTCCTCTAATTAC |
| | | ACCATGCCCGTCACCCCATGAGGGATCAGAGAAGGGATGAGTCTTCTAAACTCT |
| | | ATATTCGCTGTGAGTCCAGGTTGTAAGGGGGAGCACTGTGGATGCATCCTATTGC |
| | | ACTCCAGCTGATGACACCAAAGCTTAGGTGTTTGCTGAAAGTTCTTGATGTTGTG |
| | | ACTTACCACCCCTGCCTCACAACTGCAGACATAAGGGGACTATGGATTGCTTAGC |
| | | AGGAAAGGCACTGGTTCTCAAGGGCGGCTGCCCTTGGGAATCTTCTGGTCCCAAC |
| | | CAGAAAGACTGTGGCTTGATTTTCTCAGGTGCAGCCCAGCCGTAGGGCCTTTTCA |
| | | GAGCACCCCCTGGTTATTGCAACATTCATCAAAGTTTCTAGAACCTCTGGCCTAA |
| | | AGGAAGGGCCTGGTGGGATCTACTTGGCACTCGCTGGGGGGCCACCCCCCAGTG |
| | | CCACTCTCACTAGGCCTCTGATTGCACTTGTGTAGGATGAAGCTGGTGGGTGATG |
| | | GGAACTCAGCACCTCCCCTCAGGCAGAAAAGAATCATCTGTGGAGCTTCAAAAG |
| | | AAGGGGCCTGGAGTCTCTGCAGACCAATTCAACCCAAATCTCGGGGGCTCTTTCA |
| | | TGATTCTAATGGGCAACCAGGGTTGAAACCCTTATTTCTAGGGTCTTCAGTTGTA |
| | | CAAGACTGTGGGTCTGTACCAGAGCCCCCGTCAGAGTAGAATAAAAGGCTGGGT |
| | | AGGGTAGAGATTCCCATGTGCAGTGGAGAGAACAATCTGCAGTCACTGATAAGC |
| | | CTGAGACTTGGCTCATTTCAAAAGCGTTCAATTCATCCTCACCAGCAGTTCAGCT |
| | | GGAAAGGGGCAAATACCCCCACCTGAGCTTTGAAAACGCCCTGGGACCCTCTGC |
| | | ATTCTCTAAGTAAGTTATAGAAACCAGTCTCTTCCCTCCTTTGTGAGTGAGCTGCT |
| | | ATTCCACGTAGGCAACACCTGTTGAAATTGCCCTCAATGTCTACTCTGCATTTCTT |
| | | TCTTGTGATAAGCACACACTTTTATTGCAACATAATGATCTGCTCACATTTCCTTG |
| | | CCTGGGGGCTGTAAAACCTTACAGAACAGAAATCCTTGCCTCTTTCACCAGCCAC |
| | | ACCTGCCATACCAGGGGTACAGCTTTGTACTATTGAAGACACAGACAGGATTTTT |
| | | AAATGTAAATCTATTTTTGTAACTTTGTTGCGGGATATAGTTCTCTTTATGTAGCA |
| | | CTGAACTTTGTACAATATATTTTTAGAAACTCATTTTTCTACTAAAACAAACACA |
| | | GTTTACTTTAGAGAGACTGCAATAGAATCAAAATTTGAAACTGAAATCTTTGTTT |
| | | AAAAGGGTTAAGTTGAGGCAAGAGGAAAGCCCTTTCTCTCTCTTATAAAAAGGC |
| | | ACAACCTCATTGGGGAGCTAAGCTAGGTCATTGTCATGGTGAAGAAGAGAAGCA |
| | | TCGTTTTTATATTTAGGAAATTTTAAAAGATGATGGAAAGCACATTTAGCTTGGT |
| | | CTGAGGCAGGTTCTGTTGGGGCAGTGTTAATGGAAAGGGCTCACTGTTGTTACTA |
| | | CTAGAAAAATCCAGTTGCATGCCATACTCTCATCATCTGCCAGTGTAACCCTGTA |
| | | CATGTAAGAAAAGCAATAACATAGCACTTTGTTGGTTTATATATATAATGTGACT |
| | | TCAATGCAAATTTTATTTTTATATTTACAATTGATATGCATTTACCAGTATAAACT |
| | | AGACATGTCTGGAGAGCCTAATAATGTTCAGCACACTTTGGTTAGTTCACCAACA |
| | | GTCTTACCAAGCCTGGGCCCAGCCACCCTAGAGAAGTTATTCAGCCCTGGCTGCA |
| | | GTGACATCACCTGAGGAGCTTTTAAAAGCTTGAAGCCCAGCTACACCTCAGACC |
| | | GATTAAACGCAAATCTCTGGGGCTGAAACCCAAGCATTCGTAGTTTTTAAAGCTC |
| | | CTGAGGTCATTCCAATGTGCGGCCAAAGTTGAGAACTACTGGCCTAGGGATTAG |
| | | CCACAAGGACATGGACTTGGAGGCAAATTCTGCAGGTGTATGTGATTCTCAGGC |
| | | CTAGAGAGCTAAGACACAAAGACCTCCACATCTGTCGCTGAGAGTCAAGAACCT |
| | | GAACAGAGTTTCCATGAAGGTTCTCCAAGCACTAGAAGGGAGAGTGTCTAAACA |
| | | ATGGTTGAAAAGCAAAGGAAATATAAAACAGACACCTCTTTCCATTTCCTAAGG |
| | | TTTCTCTCTTTATTAAGGGTGGACTAGTAATAAAATATAATATTCTTGCTGCTTAT |
| | | GCAGCTGACATTGTTGCCCTCCCTAAAGCAACCAAGTAGCCTTTATTTCCCACAG |
| | | TGAAAGAAAACGCTGGCCTATCAGTTACATTACAAAAGGCAGATTTCAAGAGGA |
| | | TTGAGTAAGTAGTTGGATGGCTTTCATAAAAACAAGAATTCAAGAAGAGGATTC |
| | | ATGCTTTAAGAAACATTTGTTATACATTCCTCACAAATTATACCTGGGATAAAAA |
| | | CTATGTAGCAGGCAGTGTGTTTTCCTTCCATGTCTCTCTGCACTACCTGCAGTGTG |
| | | TCCTCTGAGGCTGCAAGTCTGTCCTATCTGAATTCCCAGCAGAAGCACTAAGAAG |
| | | CTCCACCCTATCACCTAGCAGATAAAACTATGGGGAAAACTTAAATCTGTGCATA |
| | | CATTTCTGGATGCATTTACTTATCTTTAAAAAAAAAGGAATCCTATGACCTGATT |
| | | TGGCCACAAAAATAATCTTGCTGTACAATACAATCTCTTGGAAATTAAGAGATCC |
| | | TATGGATTTGATGACTGGTATTAGAGGTGACAATGTAACCGATTAACAACAGAC |
| | | AGCAATAACTTCGTTTTAGAAACATTCAAGCAATAGCTTTATAGCTTCAACATAT |
| | | GGTACGTTTTAACCTTGAAAGTTTTGCAATGATGAAAGCAGTATTTGTACAAATG |
| | | AAAAGCAGAATTCTCTTTTATATGGTTTATACTGTTGATCAGAAATGTTGATTGT |
| | | GCATTGAGTATTAAAAAATTAGATGTATATTATTCATTGTTCTTTACTCCTGAGTA |
| | | CCTTATAATAATAATAATGTATTCTTTGTTAACAA |
| SEQ | EGFR | AGACGTCCGGGCAGCCCCCGGCGCAGCGCGGCCGCAGCAGCCTCCGCCCCCCGC |
| ID | | ACGGTGTGAGCGCCCGACGCGGCCGAGGCGGCCGGAGTCCCGAGCTAGCCCCGG |
| NO: | | CGGCCGCCGCCGCCCAGACCGGACGACAGGCCACCTCGTCGGCGTCCGCCCGAG |
| 416 | | TCCCCGCCTCGCCGCCAACGCCACAACCACCGCGCACGGCCCCCTGACTCCGTCC |
| | | AGTATTGATCGGGAGAGCCGGAGCGAGCTCTTCGGGGAGCAGCGATGCGACCCT |
| | | CCGGGACGGCCGGGGCAGCGCTCCTGGCGCTGCTGGCTGCGCTCTGCCCGGCGA |
| | | GTCGGGCTCTGGAGGAAAAGAAAGGTAATTATGTGGTGACAGATCACGGCTCGT |
| | | GCGTCCGAGCCTGTGGGGCCGACAGCTATGAGATGGAGGAAGACGGCGTCCGCA |
| | | AGTGTAAGAAGTGCGAAGGGCCTTGCCGCAAAGTGTGTAACGGAATAGGTATTG |
| | | GTGAATTTAAAGACTCACTCTCCATAAATGCTACGAATATTAAACACTTCAAAAA |
| | | CTGCACCTCCATCAGTGGCGATCTCCACATCCTGCCGGTGGCATTTAGGGGTGAC |
| | | TCCTTCACACATACTCCTCCTCTGGATCCACAGGAACTGGATATTCTGAAAACCG |
| | | TAAAGGAAATCACAGGGTTTTTGCTGATTCAGGCTTGGCCTGAAAACAGGACGG |
| | | ACCTCCATGCCTTTGAGAACCTAGAAATCATACGCGGCAGGACCAAGCAACATG |
| | | GTCAGTTTTCTCTTGCAGTCGTCAGCCTGAACATAACATCCTTGGGATTACGCTCC |
| | | CTCAAGGAGATAAGTGATGGAGATGTGATAATTTCAGGAAACAAAAATTTGTGC |
| | | TATGCAAATACAATAAACTGGAAAAAACTGTTTGGGACCTCCGGTCAGAAAACC |
| | | AAAATTATAAGCAACAGAGGTGAAAACAGCTGCAAGGCCACAGGCCAGGTCTGC |
| | | CATGCCTTGTGCTCCCCCGAGGGCTGCTGGGGCCCGGAGCCCAGGGACTGCGTCT |
| | | CTTGCCGGAATGTCAGCCGAGGCAGGGAATGCGTGGACAAGTGCAACCTTCTGG |
| | | AGGGTGAGCCAAGGGAGTTTGTGGAGAACTCTGAGTGCATACAGTGCCACCCAG |
| | | AGTGCCTGCCTCAGGCCATGAACATCACCTGCACAGGACGGGGACCAGACAACT |
| | | GTATCCAGTGTGCCCACTACATTGACGGCCCCCACTGCGTCAAGACCTGCCCGGC |
| | | AGGAGTCATGGGAGAAAACAACACCCTGGTCTGGAAGTACGCAGACGCCGGCCA |
| | | TGTGTGCCACCTGTGCCATCCAAACTGCACCTACGGATGCACTGGGCCAGGTCTT |
| | | GAAGGCTGTCCAACGAATGGGCCTAAGATCCCGTCCATCGCCACTGGGATGGTG |
| | | GGGGCCCTCCTCTTGCTGCTGGTGGTGGCCCTGGGGATCGGCCTCTTCATGCGAA |
| | | GGCGCCACATCGTTCGGAAGCGCACGCTGCGGAGGCTGCTGCAGGAGAGGGAGC |
| | | TTGTGGAGCCTCTTACACCCAGTGGAGAAGCTCCCAACCAAGCTCTCTTGAGGAT |
| | | CTTGAAGGAAACTGAATTCAAAAAGATCAAAGTGCTGGGCTCCGGTGCGTTCGG |
| | | CACGGTGTATAAGGGACTCTGGATCCCAGAAGGTGAGAAAGTTAAAATTCCCGT |
| | | CGCTATCAAGGAATTAAGAGAAGCAACATCTCCGAAAGCCAACAAGGAAATCCT |
| | | CGATGAAGCCTACGTGATGGCCAGCGTGGACAACCCCCACGTGTGCCGCCTGCT |
| | | GGGCATCTGCCTCACCTCCACCGTGCAGCTCATCACGCAGCTCATGCCCTTCGGC |
| | | TGCCTCCTGGACTATGTCCGGGAACACAAAGACAATATTGGCTCCCAGTACCTGC |
| | | TCAACTGGTGTGTGCAGATCGCAAAGGGCATGAACTACTTGGAGGACCGTCGCT |
| | | TGGTGCACCGCGACCTGGCAGCCAGGAACGTACTGGTGAAAACACCGCAGCATG |
| | | TCAAGATCACAGATTTTGGGCTGGCCAAACTGCTGGGTGCGGAAGAGAAAGAAT |
| | | ACCATGCAGAAGGAGGCAAAGTGCCTATCAAGTGGATGGCATTGGAATCAATTT |
| | | TACACAGAATCTATACCCACCAGAGTGATGTCTGGAGCTACGGGGTGACTGTTTG |
| | | GGAGTTGATGACCTTTGGATCCAAGCCATATGACGGAATCCCTGCCAGCGAGAT |
| | | CTCCTCCATCCTGGAGAAAGGAGAACGCCTCCCTCAGCCACCCATATGTACCATC |
| | | GATGTCTACATGATCATGGTCAAGTGCTGGATGATAGACGCAGATAGTCGCCCA |
| | | AAGTTCCGTGAGTTGATCATCGAATTCTCCAAAATGGCCCGAGACCCCCAGCGCT |
| | | ACCTTGTCATTCAGGGGGATGAAAGAATGCATTTGCCAAGTCCTACAGACTCCAA |
| | | CTTCTACCGTGCCCTGATGGATGAAGAAGACATGGACGACGTGGTGGATGCCGA |
| | | CGAGTACCTCATCCCACAGCAGGGCTTCTTCAGCAGCCCCTCCACGTCACGGACT |
| | | CCCCTCCTGAGCTCTCTGAGTGCAACCAGCAACAATTCCACCGTGGCTTGCATTG |
| | | ATAGAAATGGGCTGCAAAGCTGTCCCATCAAGGAAGACAGCTTCTTGCAGCGAT |
| | | ACAGCTCAGACCCCACAGGCGCCTTGACTGAGGACAGCATAGACGACACCTTCC |
| | | TCCCAGTGCCTGAATACATAAACCAGTCCGTTCCCAAAAGGCCCGCTGGCTCTGT |
| | | GCAGAATCCTGTCTATCACAATCAGCCTCTGAACCCCGCGCCCAGCAGAGACCC |
| | | ACACTACCAGGACCCCCACAGCACTGCAGTGGGCAACCCCGAGTATCTCAACAC |
| | | TGTCCAGCCCACCTGTGTCAACAGCACATTCGACAGCCCTGCCCACTGGGCCCAG |
| | | AAAGGCAGCCACCAAATTAGCCTGGACAACCCTGACTACCAGCAGGACTTCTTT |
| | | CCCAAGGAAGCCAAGCCAAATGGCATCTTTAAGGGCTCCACAGCTGAAAATGCA |
| | | GAATACCTAAGGGTCGCGCCACAAAGCAGTGAATTTATTGGAGCATGACCACGG |
| | | AGGATAGTATGAGCCCTAAAAATCCAGACTCTTTCGATACCCAGGACCAAGCCA |
| | | CAGCAGGTCCTCCATCCCAACAGCCATGCCCGCATTAGCTCTTAGACCCACAGAC |
| | | TGGTTTTGCAACGTTTACACCGACTAGCCAGGAAGTACTTCCACCTCGGGCACAT |
| | | TTTGGGAAGTTGCATTCCTTTGTCTTCAAACTGTGAAGCATTTACAGAAACGCAT |
| | | CCAGCAAGAATATTGTCCCTTTGAGCAGAAATTTATCTTTCAAAGAGGTATATTT |
| | | GAAAAAAAAAAAAAGTATATGTGAGGATTTTTATTGATTGGGGATCTTGGAGTTT |
| | | TTCATTGTCGCTATTGATTTTTACTTCAATGGGCTCTTCCAACAAGGAAGAAGCTT |
| | | GCTGGTAGCACTTGCTACCCTGAGTTCATCCAGGCCCAACTGTGAGCAAGGAGC |
| | | ACAAGCCACAAGTCTTCCAGAGGATGCTTGATTCCAGTGGTTCTGCTTCAAGGCT |
| | | TCCACTGCAAAACACTAAAGATCCAAGAAGGCCTTCATGGCCCCAGCAGGCCGG |
| | | ATCGGTACTGTATCAAGTCATGGCAGGTACAGTAGGATAAGCCACTCTGTCCCTT |
| | | CCTGGGCAAAGAAGAAACGGAGGGGATGGAATTCTTCCTTAGACTTACTTTTGTA |
| | | AAAATGTCCCCACGGTACTTACTCCCCACTGATGGACCAGTGGTTTCCAGTCATG |
| | | AGCGTTAGACTGACTTGTTTGTCTTCCATTCCATTGTTTTGAAACTCAGTATGCTG |
| | | CCCCTGTCTTGCTGTCATGAAATCAGCAAGAGAGGATGACACATCAAATAATAA |
| | | CTCGGATTCCAGCCCACATTGGATTCATCAGCATTTGGACCAATAGCCCACAGCT |
| | | GAGAATGTGGAATACCTAAGGATAGCACCGCTTTTGTTCTCGCAAAAACGTATCT |
| | | CCTAATTTGAGGCTCAGATGAAATGCATCAGGTCCTTTGGGGCATAGATCAGAA |
| | | GACTACAAAAATGAAGCTGCTCTGAAATCTCCTTTAGCCATCACCCCAACCCCCC |
| | | AAAATTAGTTTGTGTTACTTATGGAAGATAGTTTTCTCCTTTTACTTCACTTCAAA |
| | | AGCTTTTTACTCAAAGAGTATATGTTCCCTCCAGGTCAGCTGCCCCCAAACCCCC |
| | | TCCTTACGCTTTGTCACACAAAAAGTGTCTCTGCCTTGAGTCATCTATTCAAGCAC |
| | | TTACAGCTCTGGCCACAACAGGGCATTTTACAGGTGCGAATGACAGTAGCATTAT |
| | | GAGTAGTGTGGAATTCAGGTAGTAAATATGAAACTAGGGTTTGAAATTGATAAT |
| | | GCTTTCACAACATTTGCAGATGTTTTAGAAGGAAAAAAGTTCCTTCCTAAAATAA |
| | | TTTCTCTACAATTGGAAGATTGGAAGATTCAGCTAGTTAGGAGCCCACCTTTTTT |
| | | CCTAATCTGTGTGTGCCCTGTAACCTGACTGGTTAACAGCAGTCCTTTGTAAACA |
| | | GTGTTTTAAACTCTCCTAGTCAATATCCACCCCATCCAATTTATCAAGGAAGAAA |
| | | TGGTTCAGAAAATATTTTCAGCCTACAGTTATGTTCAGTCACACACACATACAAA |
| | | ATGTTCCTTTTGCTTTTAAAGTAATTTTTGACTCCCAGATCAGTCAGAGCCCCTAC |
| | | AGCATTGTTAAGAAAGTATTTGATTTTTGTCTCAATGAAAATAAAACTATATTCA |
| | | TTTCCACTCTATTATGCTCTCAAATACCCCTAAGCATCTATACTAGCCTGGTATGG |
| | | GTATGAAAGATACAAAGATAAATAAAACATAGTCCCTGATTCTAAGAAATTCAC |
| | | AATTTAGCAAAGGAAATGGACTCATAGATGCTAACCTTAAAACAACGTGACAAA |
| | | TGCCAGACAGGACCCATCAGCCAGGCACTGTGAGAGCACAGAGCAGGGAGGTTG |
| | | GGTCCTGCCTGAGGAGACCTGGAAGGGAGGCCTCACAGGAGGATGACCAGGTCT |
| | | CAGTCAGCGGGGAGGTGGAAAGTGCAGGTGCATCAGGGGCACCCTGACCGAGG |
| | | AAACAGCTGCCAGAGGCCTCCACTGCTAAAGTCCACATAAGGCTGAGGTCAGTC |
| | | ACCCTAAACAACCTGCTCCCTCTAAGCCAGGGGATGAGCTTGGAGCATCCCACA |
| | | AGTTCCCTAAAAGTTGCAGCCCCCAGGGGGATTTTGAGCTATCATCTCTGCACAT |
| | | GCTTAGTGAGAAGACTACACAACATTTCTAAGAATCTGAGATTTTATATTGTCAG |
| | | TTAACCACTTTCATTATTCATTCACCTCAGGACATGCAGAAATATTTCAGTCAGA |
| | | ACTGGGAAACAGAAGGACCTACATTCTGCTGTCACTTATGTGTCAAGAAGCAGA |
| | | TGATCGATGAGGCAGGTCAGTTGTAAGTGAGTCACATTGTAGCATTAAATTCTAG |
| | | TATTTTTGTAGTTTGAAACAGTAACTTAATAAAAGAGCAAAAGCTATTCTAGCTT |
| | | TCTTCTTCATATTTTAATTTTCCACCATAAAGTTTAGTTGCTAAATTCTATTAATTT |
| | | TAAGATTGTGCTTCCCAAAATAGTTCTCACTTCATCTGTCCAGGGAGGCACAGTT |
| | | CTGTCTGGTAGAAGCCGCAAAGCCCTTAGCCTCTTCACGGATCTGGCGACTGTGA |
| | | TGGGCAGGTCAGGAGAGGAGCTGCCCAAAGTCCCATGATTTTCACCTAACAGCC |
| | | CTGATCAGTCAGTACTCAAAGCTTGGACTCCATCCCTGAAGGTCTTCCTGATTGA |
| | | TAGCCTGGCCTTAATACCCTACAGAAAGCCTGTCCATTGGCTGTTTCTTCCTCAGT |
| | | CAGTTCCTGGAAGACCTTACCCCATGACCCCAGCTTCAGATGTGGTCTTTGGAAA |
| | | CAGAGGTCGAAGGAAAGTAAGGAGCTGAGAGCTCACATTCATAGGTGCCGCCAG |
| | | CCTTCGTGCATCTTCTTGCATCATCTCTAAGGAGCTCCTCTAATTACACCATGCCC |
| | | GTCACCCCATGAGGGATCAGAGAAGGGATGAGTCTTCTAAACTCTATATTCGCTG |
| | | TGAGTCCAGGTTGTAAGGGGGAGCACTGTGGATGCATCCTATTGCACTCCAGCTG |
| | | ATGACACCAAAGCTTAGGTGTTTGCTGAAAGTTCTTGATGTTGTGACTTACCACC |
| | | CCTGCCTCACAACTGCAGACATAAGGGGACTATGGATTGCTTAGCAGGAAAGGC |
| | | ACTGGTTCTCAAGGGCGGCTGCCCTTGGGAATCTTCTGGTCCCAACCAGAAAGAC |
| | | TGTGGCTTGATTTTCTCAGGTGCAGCCCAGCCGTAGGGCCTTTTCAGAGCACCCC |
| | | CTGGTTATTGCAACATTCATCAAAGTTTCTAGAACCTCTGGCCTAAAGGAAGGGC |
| | | CTGGTGGGATCTACTTGGCACTCGCTGGGGGGCCACCCCCCAGTGCCACTCTCAC |
| | | TAGGCCTCTGATTGCACTTGTGTAGGATGAAGCTGGTGGGTGATGGGAACTCAGC |
| | | ACCTCCCCTCAGGCAGAAAAGAATCATCTGTGGAGCTTCAAAAGAAGGGGCCTG |
| | | GAGTCTCTGCAGACCAATTCAACCCAAATCTCGGGGGCTCTTTCATGATTCTAAT |
| | | GGGCAACCAGGGTTGAAACCCTTATTTCTAGGGTCTTCAGTTGTACAAGACTGTG |
| | | GGTCTGTACCAGAGCCCCCGTCAGAGTAGAATAAAAGGCTGGGTAGGGTAGAGA |
| | | TTCCCATGTGCAGTGGAGAGAACAATCTGCAGTCACTGATAAGCCTGAGACTTG |
| | | GCTCATTTCAAAAGCGTTCAATTCATCCTCACCAGCAGTTCAGCTGGAAAGGGGC |
| | | AAATACCCCCACCTGAGCTTTGAAAACGCCCTGGGACCCTCTGCATTCTCTAAGT |
| | | AAGTTATAGAAACCAGTCTCTTCCCTCCTTTGTGAGTGAGCTGCTATTCCACGTA |
| | | GGCAACACCTGTTGAAATTGCCCTCAATGTCTACTCTGCATTTCTTTCTTGTGATA |
| | | AGCACACACTTTTATTGCAACATAATGATCTGCTCACATTTCCTTGCCTGGGGGC |
| | | TGTAAAACCTTACAGAACAGAAATCCTTGCCTCTTTCACCAGCCACACCTGCCAT |
| | | ACCAGGGGTACAGCTTTGTACTATTGAAGACACAGACAGGATTTTTAAATGTAA |
| | | ATCTATTTTTGTAACTTTGTTGCGGGATATAGTTCTCTTTATGTAGCACTGAACTT |
| | | TGTACAATATATTTTTAGAAACTCATTTTTCTACTAAAACAAACACAGTTTACTTT |
| | | AGAGAGACTGCAATAGAATCAAAATTTGAAACTGAAATCTTTGTTTAAAAGGGT |
| | | TAAGTTGAGGCAAGAGGAAAGCCCTTTCTCTCTCTTATAAAAAGGCACAACCTCA |
| | | TTGGGGAGCTAAGCTAGGTCATTGTCATGGTGAAGAAGAGAAGCATCGTTTTTAT |
| | | ATTTAGGAAATTTTAAAAGATGATGGAAAGCACATTTAGCTTGGTCTGAGGCAG |
| | | GTTCTGTTGGGGCAGTGTTAATGGAAAGGGCTCACTGTTGTTACTACTAGAAAAA |
| | | TCCAGTTGCATGCCATACTCTCATCATCTGCCAGTGTAACCCTGTACATGTAAGA |
| | | AAAGCAATAACATAGCACTTTGTTGGTTTATATATATAATGTGACTTCAATGCAA |
| | | ATTTTATTTTTATATTTACAATTGATATGCATTTACCAGTATAAACTAGACATGTC |
| | | TGGAGAGCCTAATAATGTTCAGCACACTTTGGTTAGTTCACCAACAGTCTTACCA |
| | | AGCCTGGGCCCAGCCACCCTAGAGAAGTTATTCAGCCCTGGCTGCAGTGACATC |
| | | ACCTGAGGAGCTTTTAAAAGCTTGAAGCCCAGCTACACCTCAGACCGATTAAAC |
| | | GCAAATCTCTGGGGCTGAAACCCAAGCATTCGTAGTTTTTAAAGCTCCTGAGGTC |
| | | ATTCCAATGTGCGGCCAAAGTTGAGAACTACTGGCCTAGGGATTAGCCACAAGG |
| | | ACATGGACTTGGAGGCAAATTCTGCAGGTGTATGTGATTCTCAGGCCTAGAGAG |
| | | CTAAGACACAAAGACCTCCACATCTGTCGCTGAGAGTCAAGAACCTGAACAGAG |
| | | TTTCCATGAAGGTTCTCCAAGCACTAGAAGGGAGAGTGTCTAAACAATGGTTGA |
| | | AAAGCAAAGGAAATATAAAACAGACACCTCTTTCCATTTCCTAAGGTTTCTCTCT |
| | | TTATTAAGGGTGGACTAGTAATAAAATATAATATTCTTGCTGCTTATGCAGCTGA |
| | | CATTGTTGCCCTCCCTAAAGCAACCAAGTAGCCTTTATTTCCCACAGTGAAAGAA |
| | | AACGCTGGCCTATCAGTTACATTACAAAAGGCAGATTTCAAGAGGATTGAGTAA |
| | | GTAGTTGGATGGCTTTCATAAAAACAAGAATTCAAGAAGAGGATTCATGCTTTA |
| | | AGAAACATTTGTTATACATTCCTCACAAATTATACCTGGGATAAAAACTATGTAG |
| | | CAGGCAGTGTGTTTTCCTTCCATGTCTCTCTGCACTACCTGCAGTGTGTCCTCTGA |
| | | GGCTGCAAGTCTGTCCTATCTGAATTCCCAGCAGAAGCACTAAGAAGCTCCACCC |
| | | TATCACCTAGCAGATAAAACTATGGGGAAAACTTAAATCTGTGCATACATTTCTG |
| | | GATGCATTTACTTATCTTTAAAAAAAAAGGAATCCTATGACCTGATTTGGCCACA |
| | | AAAATAATCTTGCTGTACAATACAATCTCTTGGAAATTAAGAGATCCTATGGATT |
| | | TGATGACTGGTATTAGAGGTGACAATGTAACCGATTAACAACAGACAGCAATAA |
| | | CTTCGTTTTAGAAACATTCAAGCAATAGCTTTATAGCTTCAACATATGGTACGTTT |
| | | TAACCTTGAAAGTTTTGCAATGATGAAAGCAGTATTTGTACAAATGAAAAGCAG |
| | | AATTCTCTTTTATATGGTTTATACTGTTGATCAGAAATGTTGATTGTGCATTGAGT |
| | | ATTAAAAAATTAGATGTATATTATTCATTGTTCTTTACTCCTGAGTACCTTATAAT |
| | | AATAATAATGTATTCTTTGTTAACAA |
| SEQ | EGFR | AGACGTCCGGGCAGCCCCCGGCGCAGCGCGGCCGCAGCAGCCTCCGCCCCCCGC |
| ID | | ACGGTGTGAGCGCCCGACGCGGCCGAGGCGGCCGGAGTCCCGAGCTAGCCCCGG |
| NO: | | CGGCCGCCGCCGCCCAGACCGGACGACAGGCCACCTCGTCGGCGTCCGCCCGAG |
| 417 | | TCCCCGCCTCGCCGCCAACGCCACAACCACCGCGCACGGCCCCCTGACTCCGTCC |
| | | AGTATTGATCGGGAGAGCCGGAGCGAGCTCTTCGGGGAGCAGCGATGCGACCCT |
| | | CCGGGACGGCCGGGGCAGCGCTCCTGGCGCTGCTGGCTGCGCTCTGCCCGGCGA |
| | | GTCGGGCTCTGGAGGAAAAGAAAGTTTGCCAAGGCACGAGTAACAAGCTCACGC |
| | | AGTTGGGCACTTTTGAAGATCATTTTCTCAGCCTCCAGAGGATGTTCAATAACTG |
| | | TGAGGTGGTCCTTGGGAATTTGGAAATTACCTATGTGCAGAGGAATTATGATCTT |
| | | TCCTTCTTAAAGACCATCCAGGAGGTGGCTGGTTATGTCCTCATTGCCCTCAACA |
| | | CAGTGGAGCGAATTCCTTTGGAAAACCTGCAGATCATCAGAGGAAATATGTACT |
| | | ACGAAAATTCCTATGCCTTAGCAGTCTTATCTAACTATGATGCAAATAAAACCGG |
| | | ACTGAAGGAGCTGCCCATGAGAAATTTACAGGAAATCCTGCATGGCGCCGTGCG |
| | | GTTCAGCAACAACCCTGCCCTGTGCAACGTGGAGAGCATCCAGTGGCGGGACAT |
| | | AGTCAGCAGTGACTTTCTCAGCAACATGTCGATGGACTTCCAGAACCACCTGGGC |
| | | AGCTGCCAAAAGTGTGATCCAAGCTGTCCCAATGGGAGCTGCTGGGGTGCAGGA |
| | | GAGGAGAACTGCCAGAAACTGACCAAAATCATCTGTGCCCAGCAGTGCTCCGGG |
| | | CGCTGCCGTGGCAAGTCCCCCAGTGACTGCTGCCACAACCAGTGTGCTGCAGGCT |
| | | GCACAGGCCCCCGGGAGAGCGACTGCCTGGTCTGCCGCAAATTCCGAGACGAAG |
| | | CCACGTGCAAGGACACCTGCCCCCCACTCATGCTCTACAACCCCACCACGTACCA |
| | | GATGGATGTGAACCCCGAGGGCAAATACAGCTTTGGTGCCACCTGCGTGAAGAA |
| | | GTGTCCCCGTAATTATGTGGTGACAGATCACGGCTCGTGCGTCCGAGCCTGTGGG |
| | | GCCGACAGCTATGAGATGGAGGAAGACGGCGTCCGCAAGTGTAAGAAGTGCGA |
| | | AGGGCCTTGCCGCAAAGTGTGTAACGGAATAGGTATTGGTGAATTTAAAGACTC |
| | | ACTCTCCATAAATGCTACGAATATTAAACACTTCAAAAACTGCACCTCCATCAGT |
| | | GGCGATCTCCACATCCTGCCGGTGGCATTTAGGGGTGACTCCTTCACACATACTC |
| | | CTCCTCTGGATCCACAGGAACTGGATATTCTGAAAACCGTAAAGGAAATCACAG |
| | | GGTTTTTGCTGATTCAGGCTTGGCCTGAAAACAGGACGGACCTCCATGCCTTTGA |
| | | GAACCTAGAAATCATACGCGGCAGGACCAAGCAACATGGTCAGTTTTCTCTTGC |
| | | AGTCGTCAGCCTGAACATAACATCCTTGGGATTACGCTCCCTCAAGGAGATAAGT |
| | | GATGGAGATGTGATAATTTCAGGAAACAAAAATTTGTGCTATGCAAATACAATA |
| | | AACTGGAAAAAACTGTTTGGGACCTCCGGTCAGAAAACCAAAATTATAAGCAAC |
| | | AGAGGTGAAAACAGCTGCAAGGCCACAGGCCAGGTCTGCCATGCCTTGTGCTCC |
| | | CCCGAGGGCTGCTGGGGCCCGGAGCCCAGGGACTGCGTCTCTTGCCGGAATGTC |
| | | AGCCGAGGCAGGGAATGCGTGGACAAGTGCAACCTTCTGGAGGGTGAGCCAAG |
| | | GGAGTTTGTGGAGAACTCTGAGTGCATACAGTGCCACCCAGAGTGCCTGCCTCA |
| | | GGCCATGAACATCACCTGCACAGGACGGGGACCAGACAACTGTATCCAGTGTGC |
| | | CCACTACATTGACGGCCCCCACTGCGTCAAGACCTGCCCGGCAGGAGTCATGGG |
| | | AGAAAACAACACCCTGGTCTGGAAGTACGCAGACGCCGGCCATGTGTGCCACCT |
| | | GTGCCATCCAAACTGCACCTACGGATGCACTGGGCCAGGTCTTGAAGGCTGTCCA |
| | | ACGAATGGGCCTAAGATCCCGTCCATCGCCACTGGGATGGTGGGGGCCCTCCTCT |
| | | TGCTGCTGGTGGTGGCCCTGGGGATCGGCCTCTTCATGCGAAGGCGCCACATCGT |
| | | TCGGAAGCGCACGCTGCGGAGGCTGCTGCAGGAGAGGGAGCTTGTGGAGCCTCT |
| | | TACACCCAGTGGAGAAGCTCCCAACCAAGCTCTCTTGAGGATCTTGAAGGAAAC |
| | | TGAATTCAAAAAGATCAAAGTGCTGGGCTCCGGTGCGTTCGGCACGGTGTATAA |
| | | GGGACTCTGGATCCCAGAAGGTGAGAAAGTTAAAATTCCCGTCGCTATCAAGGA |
| | | ATTAAGAGAAGCAACATCTCCGAAAGCCAACAAGGAAATCCTCGATGAAGCCTA |
| | | CGTGATGGCCAGCGTGGACAACCCCCACGTGTGCCGCCTGCTGGGCATCTGCCTC |
| | | ACCTCCACCGTGCAGCTCATCACGCAGCTCATGCCCTTCGGCTGCCTCCTGGACT |
| | | ATGTCCGGGAACACAAAGACAATATTGGCTCCCAGTACCTGCTCAACTGGTGTGT |
| | | GCAGATCGCAAAGGGCATGAACTACTTGGAGGACCGTCGCTTGGTGCACCGCGA |
| | | CCTGGCAGCCAGGAACGTACTGGTGAAAACACCGCAGCATGTCAAGATCACAGA |
| | | TTTTGGGCTGGCCAAACTGCTGGGTGCGGAAGAGAAAGAATACCATGCAGAAGG |
| | | AGGCAAAGTGCCTATCAAGTGGATGGCATTGGAATCAATTTTACACAGAATCTAT |
| | | ACCCACCAGAGTGATGTCTGGAGCTACGGGGTGACTGTTTGGGAGTTGATGACCT |
| | | TTGGATCCAAGCCATATGACGGAATCCCTGCCAGCGAGATCTCCTCCATCCTGGA |
| | | GAAAGGAGAACGCCTCCCTCAGCCACCCATATGTACCATCGATGTCTACATGATC |
| | | ATGGTCAAGTGCTGGATGATAGACGCAGATAGTCGCCCAAAGTTCCGTGAGTTG |
| | | ATCATCGAATTCTCCAAAATGGCCCGAGACCCCCAGCGCTACCTTGTCATTCAGG |
| | | GGGATGAAAGAATGCATTTGCCAAGTCCTACAGACTCCAACTTCTACCGTGCCCT |
| | | GATGGATGAAGAAGACATGGACGACGTGGTGGATGCCGACGAGTACCTCATCCC |
| | | ACAGCAGGGCTTCTTCAGCAGCCCCTCCACGTCACGGACTCCCCTCCTGAGCTCT |
| | | CTGAGTGCAACCAGCAACAATTCCACCGTGGCTTGCATTGATAGAAATGGGCTG |
| | | CAAAGCTGTCCCATCAAGGAAGACAGCTTCTTGCAGCGATACAGCTCAGACCCC |
| | | ACAGGCGCCTTGACTGAGGACAGCATAGACGACACCTTCCTCCCAGTGCCTGGT |
| | | GAGTGGCTTGTCTGGAAACAGTCCTGCTCCTCAACCTCCTCGACCCACTCAGCAG |
| | | CAGCCAGTCTCCAGTGTCCAAGCCAGGTGCTCCCTCCAGCATCTCCAGAGGGGG |
| | | AAACAGTGGCAGATTTGCAGACACAGTGAAGGGCGTAAGGAGCAGATAAACAC |
| | | ATGACCGAGCCTGCACAAGCTCTTTGTTGTGTCTGGTTGTTTGCTGTACCTCTGTT |
| | | GTAAGAATGAATCTGCAAAATTTCTAGCTTATGAAGCAAATCACGGACATACAC |
| | | ATCTGTGTGTGTGAGTGTTCATGATGTGTGTACATCTGTGTATGTGTGTGTGTGTA |
| | | TGTGTGTGTTTGTGACAGATTTGATCCCTGTTCTCTCTGCTGGCTCTATCTTGACC |
| | | TGTGAAACGTATATTTAACTAATTAAATATTAGTTAATATTAATAAATTTTAAGC |
| | | TTTATCCAGA |
| SEQ | EGFR | AGACGTCCGGGCAGCCCCCGGCGCAGCGCGGCCGCAGCAGCCTCCGCCCCCCGC |
| ID | | ACGGTGTGAGCGCCCGACGCGGCCGAGGCGGCCGGAGTCCCGAGCTAGCCCCGG |
| NO: | | CGGCCGCCGCCGCCCAGACCGGACGACAGGCCACCTCGTCGGCGTCCGCCCGAG |
| 418 | | TCCCCGCCTCGCCGCCAACGCCACAACCACCGCGCACGGCCCCCTGACTCCGTCC |
| | | AGTATTGATCGGGAGAGCCGGAGCGAGCTCTTCGGGGAGCAGCGATGCGACCCT |
| | | CCGGGACGGCCGGGGCAGCGCTCCTGGCGCTGCTGGCTGCGCTCTGCCCGGCGA |
| | | GTCGGGCTCTGGAGGAAAAGAAAGTTTGCCAAGGCACGAGTAACAAGCTCACGC |
| | | AGTTGGGCACTTTTGAAGATCATTTTCTCAGCCTCCAGAGGATGTTCAATAACTG |
| | | TGAGGTGGTCCTTGGGAATTTGGAAATTACCTATGTGCAGAGGAATTATGATCTT |
| | | TCCTTCTTAAAGACCATCCAGGAGGTGGCTGGTTATGTCCTCATTGCCCTCAACA |
| | | CAGTGGAGCGAATTCCTTTGGAAAACCTGCAGATCATCAGAGGAAATATGTACT |
| | | ACGAAAATTCCTATGCCTTAGCAGTCTTATCTAACTATGATGCAAATAAAACCGG |
| | | ACTGAAGGAGCTGCCCATGAGAAATTTACAGGGCCAAAAGTGTGATCCAAGCTG |
| | | TCCCAATGGGAGCTGCTGGGGTGCAGGAGAGGAGAACTGCCAGAAACTGACCAA |
| | | AATCATCTGTGCCCAGCAGTGCTCCGGGCGCTGCCGTGGCAAGTCCCCCAGTGAC |
| | | TGCTGCCACAACCAGTGTGCTGCAGGCTGCACAGGCCCCCGGGAGAGCGACTGC |
| | | CTGGTCTGCCGCAAATTCCGAGACGAAGCCACGTGCAAGGACACCTGCCCCCCA |
| | | CTCATGCTCTACAACCCCACCACGTACCAGATGGATGTGAACCCCGAGGGCAAA |
| | | TACAGCTTTGGTGCCACCTGCGTGAAGAAGTGTCCCCGTAATTATGTGGTGACAG |
| | | ATCACGGCTCGTGCGTCCGAGCCTGTGGGGCCGACAGCTATGAGATGGAGGAAG |
| | | ACGGCGTCCGCAAGTGTAAGAAGTGCGAAGGGCCTTGCCGCAAAGTGTGTAACG |
| | | GAATAGGTATTGGTGAATTTAAAGACTCACTCTCCATAAATGCTACGAATATTAA |
| | | ACACTTCAAAAACTGCACCTCCATCAGTGGCGATCTCCACATCCTGCCGGTGGCA |
| | | TTTAGGGGTGACTCCTTCACACATACTCCTCCTCTGGATCCACAGGAACTGGATA |
| | | TTCTGAAAACCGTAAAGGAAATCACAGGGTTTTTGCTGATTCAGGCTTGGCCTGA |
| | | AAACAGGACGGACCTCCATGCCTTTGAGAACCTAGAAATCATACGCGGCAGGAC |
| | | CAAGCAACATGGTCAGTTTTCTCTTGCAGTCGTCAGCCTGAACATAACATCCTTG |
| | | GGATTACGCTCCCTCAAGGAGATAAGTGATGGAGATGTGATAATTTCAGGAAAC |
| | | AAAAATTTGTGCTATGCAAATACAATAAACTGGAAAAAACTGTTTGGGACCTCC |
| | | GGTCAGAAAACCAAAATTATAAGCAACAGAGGTGAAAACAGCTGCAAGGCCAC |
| | | AGGCCAGGTCTGCCATGCCTTGTGCTCCCCCGAGGGCTGCTGGGGCCCGGAGCCC |
| | | AGGGACTGCGTCTCTTGCCGGAATGTCAGCCGAGGCAGGGAATGCGTGGACAAG |
| | | TGCAACCTTCTGGAGGGTGAGCCAAGGGAGTTTGTGGAGAACTCTGAGTGCATA |
| | | CAGTGCCACCCAGAGTGCCTGCCTCAGGCCATGAACATCACCTGCACAGGACGG |
| | | GGACCAGACAACTGTATCCAGTGTGCCCACTACATTGACGGCCCCCACTGCGTCA |
| | | AGACCTGCCCGGCAGGAGTCATGGGAGAAAACAACACCCTGGTCTGGAAGTACG |
| | | CAGACGCCGGCCATGTGTGCCACCTGTGCCATCCAAACTGCACCTACGGATGCAC |
| | | TGGGCCAGGTCTTGAAGGCTGTCCAACGAATGGGCCTAAGATCCCGTCCATCGCC |
| | | ACTGGGATGGTGGGGGCCCTCCTCTTGCTGCTGGTGGTGGCCCTGGGGATCGGCC |
| | | TCTTCATGCGAAGGCGCCACATCGTTCGGAAGCGCACGCTGCGGAGGCTGCTGC |
| | | AGGAGAGGGAGCTTGTGGAGCCTCTTACACCCAGTGGAGAAGCTCCCAACCAAG |
| | | CTCTCTTGAGGATCTTGAAGGAAACTGAATTCAAAAAGATCAAAGTGCTGGGCT |
| | | CCGGTGCGTTCGGCACGGTGTATAAGGGACTCTGGATCCCAGAAGGTGAGAAAG |
| | | TTAAAATTCCCGTCGCTATCAAGGAATTAAGAGAAGCAACATCTCCGAAAGCCA |
| | | ACAAGGAAATCCTCGATGAAGCCTACGTGATGGCCAGCGTGGACAACCCCCACG |
| | | TGTGCCGCCTGCTGGGCATCTGCCTCACCTCCACCGTGCAGCTCATCACGCAGCT |
| | | CATGCCCTTCGGCTGCCTCCTGGACTATGTCCGGGAACACAAAGACAATATTGGC |
| | | TCCCAGTACCTGCTCAACTGGTGTGTGCAGATCGCAAAGGGCATGAACTACTTGG |
| | | AGGACCGTCGCTTGGTGCACCGCGACCTGGCAGCCAGGAACGTACTGGTGAAAA |
| | | CACCGCAGCATGTCAAGATCACAGATTTTGGGCTGGCCAAACTGCTGGGTGCGG |
| | | AAGAGAAAGAATACCATGCAGAAGGAGGCAAAGTGCCTATCAAGTGGATGGCA |
| | | TTGGAATCAATTTTACACAGAATCTATACCCACCAGAGTGATGTCTGGAGCTACG |
| | | GGGTGACTGTTTGGGAGTTGATGACCTTTGGATCCAAGCCATATGACGGAATCCC |
| | | TGCCAGCGAGATCTCCTCCATCCTGGAGAAAGGAGAACGCCTCCCTCAGCCACC |
| | | CATATGTACCATCGATGTCTACATGATCATGGTCAAGTGCTGGATGATAGACGCA |
| | | GATAGTCGCCCAAAGTTCCGTGAGTTGATCATCGAATTCTCCAAAATGGCCCGAG |
| | | ACCCCCAGCGCTACCTTGTCATTCAGGGGGATGAAAGAATGCATTTGCCAAGTCC |
| | | TACAGACTCCAACTTCTACCGTGCCCTGATGGATGAAGAAGACATGGACGACGT |
| | | GGTGGATGCCGACGAGTACCTCATCCCACAGCAGGGCTTCTTCAGCAGCCCCTCC |
| | | ACGTCACGGACTCCCCTCCTGAGCTCTCTGAGTGCAACCAGCAACAATTCCACCG |
| | | TGGCTTGCATTGATAGAAATGGGCTGCAAAGCTGTCCCATCAAGGAAGACAGCT |
| | | TCTTGCAGCGATACAGCTCAGACCCCACAGGCGCCTTGACTGAGGACAGCATAG |
| | | ACGACACCTTCCTCCCAGTGCCTGGTGAGTGGCTTGTCTGGAAACAGTCCTGCTC |
| | | CTCAACCTCCTCGACCCACTCAGCAGCAGCCAGTCTCCAGTGTCCAAGCCAGGTG |
| | | CTCCCTCCAGCATCTCCAGAGGGGGAAACAGTGGCAGATTTGCAGACACAGTGA |
| | | AGGGCGTAAGGAGCAGATAAACACATGACCGAGCCTGCACAAGCTCTTTGTTGT |
| | | GTCTGGTTGTTTGCTGTACCTCTGTTGTAAGAATGAATCTGCAAAATTTCTAGCTT |
| | | ATGAAGCAAATCACGGACATACACATCTGTGTGTGTGAGTGTTCATGATGTGTGT |
| | | ACATCTGTGTATGTGTGTGTGTGTATGTGTGTGTTTGTGACAGATTTGATCCCTGT |
| | | TCTCTCTGCTGGCTCTATCTTGACCTGTGAAACGTATATTTAACTAATTAAATATT |
| | | AGTTAATATTAATAAATTTTAAGCTTTATCCAGA |
| SEQ | EGFR | AGACGTCCGGGCAGCCCCCGGCGCAGCGCGGCCGCAGCAGCCTCCGCCCCCCGC |
| ID | | ACGGTGTGAGCGCCCGACGCGGCCGAGGCGGCCGGAGTCCCGAGCTAGCCCCGG |
| NO: | | CGGCCGCCGCCGCCCAGACCGGACGACAGGCCACCTCGTCGGCGTCCGCCCGAG |
| 419 | | TCCCCGCCTCGCCGCCAACGCCACAACCACCGCGCACGGCCCCCTGACTCCGTCC |
| | | AGTATTGATCGGGAGAGCCGGAGCGAGCTCTTCGGGGAGCAGCGATGCGACCCT |
| | | CCGGGACGGCCGGGGCAGCGCTCCTGGCGCTGCTGGCTGCGCTCTGCCCGGCGA |
| | | GTCGGGCTCTGGAGGAAAAGAAAGTTTGCCAAGGCACGAGTAACAAGCTCACGC |
| | | AGTTGGGCACTTTTGAAGATCATTTTCTCAGCCTCCAGAGGATGTTCAATAACTG |
| | | TGAGGTGGTCCTTGGGAATTTGGAAATTACCTATGTGCAGAGGAATTATGATCTT |
| | | TCCTTCTTAAAGACCATCCAGGAGGTGGCTGGTTATGTCCTCATTGCCCTCAACA |
| | | CAGTGGAGCGAATTCCTTTGGAAAACCTGCAGATCATCAGAGGAAATATGTACT |
| | | ACGAAAATTCCTATGCCTTAGCAGTCTTATCTAACTATGATGCAAATAAAACCGG |
| | | ACTGAAGGAGCTGCCCATGAGAAATTTACAGGAAATCCTGCATGGCGCCGTGCG |
| | | GTTCAGCAACAACCCTGCCCTGTGCAACGTGGAGAGCATCCAGTGGCGGGACAT |
| | | AGTCAGCAGTGACTTTCTCAGCAACATGTCGATGGACTTCCAGAACCACCTGGGC |
| | | AGCTGCCAAAAGTGTGATCCAAGCTGTCCCAATGGGAGCTGCTGGGGTGCAGGA |
| | | GAGGAGAACTGCCAGAAACTGACCAAAATCATCTGTGCCCAGCAGTGCTCCGGG |
| | | CGCTGCCGTGGCAAGTCCCCCAGTGACTGCTGCCACAACCAGTGTGCTGCAGGCT |
| | | GCACAGGCCCCCGGGAGAGCGACTGCCTGGTCTGCCGCAAATTCCGAGACGAAG |
| | | CCACGTGCAAGGACACCTGCCCCCCACTCATGCTCTACAACCCCACCACGTACCA |
| | | GATGGATGTGAACCCCGAGGGCAAATACAGCTTTGGTGCCACCTGCGTGAAGAA |
| | | GTGTCCCCGTAATTATGTGGTGACAGATCACGGCTCGTGCGTCCGAGCCTGTGGG |
| | | GCCGACAGCTATGAGATGGAGGAAGACGGCGTCCGCAAGTGTAAGAAGTGCGA |
| | | AGGGCCTTGCCGCAAAGTGTGTAACGGAATAGGTATTGGTGAATTTAAAGACTC |
| | | ACTCTCCATAAATGCTACGAATATTAAACACTTCAAAAACTGCACCTCCATCAGT |
| | | GGCGATCTCCACATCCTGCCGGTGGCATTTAGGGGTGACTCCTTCACACATACTC |
| | | CTCCTCTGGATCCACAGGAACTGGATATTCTGAAAACCGTAAAGGAAATCACAG |
| | | GGTTTTTGCTGATTCAGGCTTGGCCTGAAAACAGGACGGACCTCCATGCCTTTGA |
| | | GAACCTAGAAATCATACGCGGCAGGACCAAGCAACATGGTCAGTTTTCTCTTGC |
| | | AGTCGTCAGCCTGAACATAACATCCTTGGGATTACGCTCCCTCAAGGAGATAAGT |
| | | GATGGAGATGTGATAATTTCAGGAAACAAAAATTTGTGCTATGCAAATACAATA |
| | | AACTGGAAAAAACTGTTTGGGACCTCCGGTCAGAAAACCAAAATTATAAGCAAC |
| | | AGAGGTGAAAACAGCTGCAAGGCCACAGGCCAGGTCTGCCATGCCTTGTGCTCC |
| | | CCCGAGGGCTGCTGGGGCCCGGAGCCCAGGGACTGCGTCTCTTGCCGGAATGTC |
| | | AGCCGAGGCAGGGAATGCGTGGACAAGTGCAACCTTCTGGAGGGTGAGCCAAG |
| | | GGAGTTTGTGGAGAACTCTGAGTGCATACAGTGCCACCCAGAGTGCCTGCCTCA |
| | | GGCCATGAACATCACCTGCACAGGACGGGGACCAGACAACTGTATCCAGTGTGC |
| | | CCACTACATTGACGGCCCCCACTGCGTCAAGACCTGCCCGGCAGGAGTCATGGG |
| | | AGAAAACAACACCCTGGTCTGGAAGTACGCAGACGCCGGCCATGTGTGCCACCT |
| | | GTGCCATCCAAACTGCACCTACGGGCCAGGAAATGAGAGTCTCAAAGCCATGTT |
| | | ATTCTGCCTTTTTAAACTATCATCCTGTAATCAAAGTAATGATGGCAGCGTGTCC |
| | | CACCAGAGCGGGAGCCCAGCTGCTCAGGAGTCATGCTTAGGATGGATCCCTTCTC |
| | | TTCTGCCGTCAGAGTTTCAGCTGGGTTGGGGTGGATGCAGCCACCTCCATGCCTG |
| | | GCCTTCTGCATCTGTGATCATCACGGCCTCCTCCTGCCACTGAGCCTCATGCCTTC |
| | | ACGTGTCTGTTCCCCCCGCTTTTCCTTTCTGCCACCCCTGCACGTGGGCCGCCAGG |
| | | TTCCCAAGAGTATCCTACCCATTTCCTTCCTTCCACTCCCTTTGCCAGTGCCTCTC |
| | | ACCCCAACTAGTAGCTAACCATCACCCCCAGGACTGACCTCTTCCTCCTCGCTGC |
| | | CAGATGATTGTTCAAAGCACAGAATTTGTCAGAAACCTGCAGGGACTCCATGCT |
| | | GCCAGCCTTCTCCGTAATTAGCATGGCCCCAGTCCATGCTTCTAGCCTTGGTTCCT |
| | | TCTGCCCCTCTGTTTGAAATTCTAGAGCCAGCTGTGGGACAATTATCTGTGTCAA |
| | | AAGCCAGATGTGAAAACATCTCAATAACAAACTGGCTGCTTTGTTCAATGCTAGA |
| | | ACAACGCCTGTCACAGAGTAGAAACTCAAAAATATTTGCTGAGTGAATGAACAA |
| | | ATGAATAAATGCATAATAAATAATTAACCACCAATCCAA |
| SEQ | EGFR | AGACGTCCGGGCAGCCCCCGGCGCAGCGCGGCCGCAGCAGCCTCCGCCCCCCGC |
| ID | | ACGGTGTGAGCGCCCGACGCGGCCGAGGCGGCCGGAGTCCCGAGCTAGCCCCGG |
| NO: | | CGGCCGCCGCCGCCCAGACCGGACGACAGGCCACCTCGTCGGCGTCCGCCCGAG |
| 420 | | TCCCCGCCTCGCCGCCAACGCCACAACCACCGCGCACGGCCCCCTGACTCCGTCC |
| | | AGTATTGATCGGGAGAGCCGGAGCGAGCTCTTCGGGGAGCAGCGATGCGACCCT |
| | | CCGGGACGGCCGGGGCAGCGCTCCTGGCGCTGCTGGCTGCGCTCTGCCCGGCGA |
| | | GTCGGGCTCTGGAGGAAAAGAAAGTTTGCCAAGGCACGAGTAACAAGCTCACGC |
| | | AGTTGGGCACTTTTGAAGATCATTTTCTCAGCCTCCAGAGGATGTTCAATAACTG |
| | | TGAGGTGGTCCTTGGGAATTTGGAAATTACCTATGTGCAGAGGAATTATGATCTT |
| | | TCCTTCTTAAAGACCATCCAGGAGGTGGCTGGTTATGTCCTCATTGCCCTCAACA |
| | | CAGTGGAGCGAATTCCTTTGGAAAACCTGCAGATCATCAGAGGAAATATGTACT |
| | | ACGAAAATTCCTATGCCTTAGCAGTCTTATCTAACTATGATGCAAATAAAACCGG |
| | | ACTGAAGGAGCTGCCCATGAGAAATTTACAGGAAATCCTGCATGGCGCCGTGCG |
| | | GTTCAGCAACAACCCTGCCCTGTGCAACGTGGAGAGCATCCAGTGGCGGGACAT |
| | | AGTCAGCAGTGACTTTCTCAGCAACATGTCGATGGACTTCCAGAACCACCTGGGC |
| | | AGCTGCCAAAAGTGTGATCCAAGCTGTCCCAATGGGAGCTGCTGGGGTGCAGGA |
| | | GAGGAGAACTGCCAGAAACTGACCAAAATCATCTGTGCCCAGCAGTGCTCCGGG |
| | | CGCTGCCGTGGCAAGTCCCCCAGTGACTGCTGCCACAACCAGTGTGCTGCAGGCT |
| | | GCACAGGCCCCCGGGAGAGCGACTGCCTGGTCTGCCGCAAATTCCGAGACGAAG |
| | | CCACGTGCAAGGACACCTGCCCCCCACTCATGCTCTACAACCCCACCACGTACCA |
| | | GATGGATGTGAACCCCGAGGGCAAATACAGCTTTGGTGCCACCTGCGTGAAGAA |
| | | GTGTCCCCGTAATTATGTGGTGACAGATCACGGCTCGTGCGTCCGAGCCTGTGGG |
| | | GCCGACAGCTATGAGATGGAGGAAGACGGCGTCCGCAAGTGTAAGAAGTGCGA |
| | | AGGGCCTTGCCGCAAAGTGTGTAACGGAATAGGTATTGGTGAATTTAAAGACTC |
| | | ACTCTCCATAAATGCTACGAATATTAAACACTTCAAAAACTGCACCTCCATCAGT |
| | | GGCGATCTCCACATCCTGCCGGTGGCATTTAGGGGTGACTCCTTCACACATACTC |
| | | CTCCTCTGGATCCACAGGAACTGGATATTCTGAAAACCGTAAAGGAAATCACAG |
| | | GGTTTTTGCTGATTCAGGCTTGGCCTGAAAACAGGACGGACCTCCATGCCTTTGA |
| | | GAACCTAGAAATCATACGCGGCAGGACCAAGCAACATGGTCAGTTTTCTCTTGC |
| | | AGTCGTCAGCCTGAACATAACATCCTTGGGATTACGCTCCCTCAAGGAGATAAGT |
| | | GATGGAGATGTGATAATTTCAGGAAACAAAAATTTGTGCTATGCAAATACAATA |
| | | AACTGGAAAAAACTGTTTGGGACCTCCGGTCAGAAAACCAAAATTATAAGCAAC |
| | | AGAGGTGAAAACAGCTGCAAGGCCACAGGCCAGGTCTGCCATGCCTTGTGCTCC |
| | | CCCGAGGGCTGCTGGGGCCCGGAGCCCAGGGACTGCGTCTCTTGCCGGAATGTC |
| | | AGCCGAGGCAGGGAATGCGTGGACAAGTGCAACCTTCTGGAGGGTGAGCCAAG |
| | | GGAGTTTGTGGAGAACTCTGAGTGCATACAGTGCCACCCAGAGTGCCTGCCTCA |
| | | GGCCATGAACATCACCTGCACAGGACGGGGACCAGACAACTGTATCCAGTGTGC |
| | | CCACTACATTGACGGCCCCCACTGCGTCAAGACCTGCCCGGCAGGAGTCATGGG |
| | | AGAAAACAACACCCTGGTCTGGAAGTACGCAGACGCCGGCCATGTGTGCCACCT |
| | | GTGCCATCCAAACTGCACCTACGGGTCCTAATAAATCTTCACTGTCTGACTTTAG |
| | | TCTCCCACTAAAACTGCATTTCCTTTCTACAATTTCAATTTCTCCCTTTGCTTCAA |
| | | ATAAAGTCCTGACACTATTCATTTGA |
| SEQ | EGFR | AGACGTCCGGGCAGCCCCCGGCGCAGCGCGGCCGCAGCAGCCTCCGCCCCCCGC |
| ID | | ACGGTGTGAGCGCCCGACGCGGCCGAGGCGGCCGGAGTCCCGAGCTAGCCCCGG |
| NO: | | CGGCCGCCGCCGCCCAGACCGGACGACAGGCCACCTCGTCGGCGTCCGCCCGAG |
| 421 | | TCCCCGCCTCGCCGCCAACGCCACAACCACCGCGCACGGCCCCCTGACTCCGTCC |
| | | AGTATTGATCGGGAGAGCCGGAGCGAGCTCTTCGGGGAGCAGCGATGCGACCCT |
| | | CCGGGACGGCCGGGGCAGCGCTCCTGGCGCTGCTGGCTGCGCTCTGCCCGGCGA |
| | | GTCGGGCTCTGGAGGAAAAGAAAGTTTGCCAAGGCACGAGTAACAAGCTCACGC |
| | | AGTTGGGCACTTTTGAAGATCATTTTCTCAGCCTCCAGAGGATGTTCAATAACTG |
| | | TGAGGTGGTCCTTGGGAATTTGGAAATTACCTATGTGCAGAGGAATTATGATCTT |
| | | TCCTTCTTAAAGACCATCCAGGAGGTGGCTGGTTATGTCCTCATTGCCCTCAACA |
| | | CAGTGGAGCGAATTCCTTTGGAAAACCTGCAGATCATCAGAGGAAATATGTACT |
| | | ACGAAAATTCCTATGCCTTAGCAGTCTTATCTAACTATGATGCAAATAAAACCGG |
| | | ACTGAAGGAGCTGCCCATGAGAAATTTACAGGAAATCCTGCATGGCGCCGTGCG |
| | | GTTCAGCAACAACCCTGCCCTGTGCAACGTGGAGAGCATCCAGTGGCGGGACAT |
| | | AGTCAGCAGTGACTTTCTCAGCAACATGTCGATGGACTTCCAGAACCACCTGGGC |
| | | AGCTGCCAAAAGTGTGATCCAAGCTGTCCCAATGGGAGCTGCTGGGGTGCAGGA |
| | | GAGGAGAACTGCCAGAAACTGACCAAAATCATCTGTGCCCAGCAGTGCTCCGGG |
| | | CGCTGCCGTGGCAAGTCCCCCAGTGACTGCTGCCACAACCAGTGTGCTGCAGGCT |
| | | GCACAGGCCCCCGGGAGAGCGACTGCCTGGTCTGCCGCAAATTCCGAGACGAAG |
| | | CCACGTGCAAGGACACCTGCCCCCCACTCATGCTCTACAACCCCACCACGTACCA |
| | | GATGGATGTGAACCCCGAGGGCAAATACAGCTTTGGTGCCACCTGCGTGAAGAA |
| | | GTGTCCCCGTAATTATGTGGTGACAGATCACGGCTCGTGCGTCCGAGCCTGTGGG |
| | | GCCGACAGCTATGAGATGGAGGAAGACGGCGTCCGCAAGTGTAAGAAGTGCGA |
| | | AGGGCCTTGCCGCAAAGTGTGTAACGGAATAGGTATTGGTGAATTTAAAGACTC |
| | | ACTCTCCATAAATGCTACGAATATTAAACACTTCAAAAACTGCACCTCCATCAGT |
| | | GGCGATCTCCACATCCTGCCGGTGGCATTTAGGGGTGACTCCTTCACACATACTC |
| | | CTCCTCTGGATCCACAGGAACTGGATATTCTGAAAACCGTAAAGGAAATCACAG |
| | | GTTTGAGCTGAATTATCACATGAATATAAATGGGAAATCAGTGTTTTAGAGAGA |
| | | GAACTTTTCGACATATTTCCTGTTCCCTTGGAATAAAAACATTTCTTCTGAAA |
| SEQ | EGFR | AGTTCTCCACTGGACTTCAGAACAAGAGGGAGCTCTGGGCTGCTGGCTGGTTGTG |
| ID | | CATTTGCTGTGGGTTCCCTCCGGCAGGCGACCTCTCCGCGCTGAGAAGGTTATCC |
| NO: | | GGATAACCAATTTGCCAAGGCACGAGTAACAAGCTCACGCAGTTGGGCACTTTT |
| 422 | | GAAGATCATTTTCTCAGCCTCCAGAGGATGTTCAATAACTGTGAGGTGGTCCTTG |
| | | GGAATTTGGAAATTACCTATGTGCAGAGGAATTATGATCTTTCCTTCTTAAAGAC |
| | | CATCCAGGAGGTGGCTGGTTATGTCCTCATTGCCCTCAACACAGTGGAGCGAATT |
| | | CCTTTGGAAAACCTGCAGATCATCAGAGGAAATATGTACTACGAAAATTCCTATG |
| | | CCTTAGCAGTCTTATCTAACTATGATGCAAATAAAACCGGACTGAAGGAGCTGCC |
| | | CATGAGAAATTTACAGGAAATCCTGCATGGCGCCGTGCGGTTCAGCAACAACCC |
| | | TGCCCTGTGCAACGTGGAGAGCATCCAGTGGCGGGACATAGTCAGCAGTGACTT |
| | | TCTCAGCAACATGTCGATGGACTTCCAGAACCACCTGGGCAGCTGCCAAAAGTG |
| | | TGATCCAAGCTGTCCCAATGGGAGCTGCTGGGGTGCAGGAGAGGAGAACTGCCA |
| | | GAAACTGACCAAAATCATCTGTGCCCAGCAGTGCTCCGGGCGCTGCCGTGGCAA |
| | | GTCCCCCAGTGACTGCTGCCACAACCAGTGTGCTGCAGGCTGCACAGGCCCCCG |
| | | GGAGAGCGACTGCCTGGTCTGCCGCAAATTCCGAGACGAAGCCACGTGCAAGGA |
| | | CACCTGCCCCCCACTCATGCTCTACAACCCCACCACGTACCAGATGGATGTGAAC |
| | | CCCGAGGGCAAATACAGCTTTGGTGCCACCTGCGTGAAGAAGTGTCCCCGTAATT |
| | | ATGTGGTGACAGATCACGGCTCGTGCGTCCGAGCCTGTGGGGCCGACAGCTATG |
| | | AGATGGAGGAAGACGGCGTCCGCAAGTGTAAGAAGTGCGAAGGGCCTTGCCGC |
| | | AAAGTGTGTAACGGAATAGGTATTGGTGAATTTAAAGACTCACTCTCCATAAATG |
| | | CTACGAATATTAAACACTTCAAAAACTGCACCTCCATCAGTGGCGATCTCCACAT |
| | | CCTGCCGGTGGCATTTAGGGGTGACTCCTTCACACATACTCCTCCTCTGGATCCA |
| | | CAGGAACTGGATATTCTGAAAACCGTAAAGGAAATCACAGGGTTTTTGCTGATTC |
| | | AGGCTTGGCCTGAAAACAGGACGGACCTCCATGCCTTTGAGAACCTAGAAATCA |
| | | TACGCGGCAGGACCAAGCAACATGGTCAGTTTTCTCTTGCAGTCGTCAGCCTGAA |
| | | CATAACATCCTTGGGATTACGCTCCCTCAAGGAGATAAGTGATGGAGATGTGAT |
| | | AATTTCAGGAAACAAAAATTTGTGCTATGCAAATACAATAAACTGGAAAAAACT |
| | | GTTTGGGACCTCCGGTCAGAAAACCAAAATTATAAGCAACAGAGGTGAAAACAG |
| | | CTGCAAGGCCACAGGCCAGGTCTGCCATGCCTTGTGCTCCCCCGAGGGCTGCTGG |
| | | GGCCCGGAGCCCAGGGACTGCGTCTCTTGCCGGAATGTCAGCCGAGGCAGGGAA |
| | | TGCGTGGACAAGTGCAACCTTCTGGAGGGTGAGCCAAGGGAGTTTGTGGAGAAC |
| | | TCTGAGTGCATACAGTGCCACCCAGAGTGCCTGCCTCAGGCCATGAACATCACCT |
| | | GCACAGGACGGGGACCAGACAACTGTATCCAGTGTGCCCACTACATTGACGGCC |
| | | CCCACTGCGTCAAGACCTGCCCGGCAGGAGTCATGGGAGAAAACAACACCCTGG |
| | | TCTGGAAGTACGCAGACGCCGGCCATGTGTGCCACCTGTGCCATCCAAACTGCAC |
| | | CTACGGATGCACTGGGCCAGGTCTTGAAGGCTGTCCAACGAATGGGCCTAAGAT |
| | | CCCGTCCATCGCCACTGGGATGGTGGGGGCCCTCCTCTTGCTGCTGGTGGTGGCC |
| | | CTGGGGATCGGCCTCTTCATGCGAAGGCGCCACATCGTTCGGAAGCGCACGCTG |
| | | CGGAGGCTGCTGCAGGAGAGGGAGCTTGTGGAGCCTCTTACACCCAGTGGAGAA |
| | | GCTCCCAACCAAGCTCTCTTGAGGATCTTGAAGGAAACTGAATTCAAAAAGATC |
| | | AAAGTGCTGGGCTCCGGTGCGTTCGGCACGGTGTATAAGGGACTCTGGATCCCA |
| | | GAAGGTGAGAAAGTTAAAATTCCCGTCGCTATCAAGGAATTAAGAGAAGCAACA |
| | | TCTCCGAAAGCCAACAAGGAAATCCTCGATGAAGCCTACGTGATGGCCAGCGTG |
| | | GACAACCCCCACGTGTGCCGCCTGCTGGGCATCTGCCTCACCTCCACCGTGCAGC |
| | | TCATCACGCAGCTCATGCCCTTCGGCTGCCTCCTGGACTATGTCCGGGAACACAA |
| | | AGACAATATTGGCTCCCAGTACCTGCTCAACTGGTGTGTGCAGATCGCAAAGGG |
| | | CATGAACTACTTGGAGGACCGTCGCTTGGTGCACCGCGACCTGGCAGCCAGGAA |
| | | CGTACTGGTGAAAACACCGCAGCATGTCAAGATCACAGATTTTGGGCTGGCCAA |
| | | ACTGCTGGGTGCGGAAGAGAAAGAATACCATGCAGAAGGAGGCAAAGTGCCTA |
| | | TCAAGTGGATGGCATTGGAATCAATTTTACACAGAATCTATACCCACCAGAGTGA |
| | | TGTCTGGAGCTACGGGGTGACTGTTTGGGAGTTGATGACCTTTGGATCCAAGCCA |
| | | TATGACGGAATCCCTGCCAGCGAGATCTCCTCCATCCTGGAGAAAGGAGAACGC |
| | | CTCCCTCAGCCACCCATATGTACCATCGATGTCTACATGATCATGGTCAAGTGCT |
| | | GGATGATAGACGCAGATAGTCGCCCAAAGTTCCGTGAGTTGATCATCGAATTCTC |
| | | CAAAATGGCCCGAGACCCCCAGCGCTACCTTGTCATTCAGGGGGATGAAAGAAT |
| | | GCATTTGCCAAGTCCTACAGACTCCAACTTCTACCGTGCCCTGATGGATGAAGAA |
| | | GACATGGACGACGTGGTGGATGCCGACGAGTACCTCATCCCACAGCAGGGCTTC |
| | | TTCAGCAGCCCCTCCACGTCACGGACTCCCCTCCTGAGCTCTCTGAGTGCAACCA |
| | | GCAACAATTCCACCGTGGCTTGCATTGATAGAAATGGGCTGCAAAGCTGTCCCAT |
| | | CAAGGAAGACAGCTTCTTGCAGCGATACAGCTCAGACCCCACAGGCGCCTTGAC |
| | | TGAGGACAGCATAGACGACACCTTCCTCCCAGTGCCTGAATACATAAACCAGTC |
| | | CGTTCCCAAAAGGCCCGCTGGCTCTGTGCAGAATCCTGTCTATCACAATCAGCCT |
| | | CTGAACCCCGCGCCCAGCAGAGACCCACACTACCAGGACCCCCACAGCACTGCA |
| | | GTGGGCAACCCCGAGTATCTCAACACTGTCCAGCCCACCTGTGTCAACAGCACAT |
| | | TCGACAGCCCTGCCCACTGGGCCCAGAAAGGCAGCCACCAAATTAGCCTGGACA |
| | | ACCCTGACTACCAGCAGGACTTCTTTCCCAAGGAAGCCAAGCCAAATGGCATCTT |
| | | TAAGGGCTCCACAGCTGAAAATGCAGAATACCTAAGGGTCGCGCCACAAAGCAG |
| | | TGAATTTATTGGAGCATGACCACGGAGGATAGTATGAGCCCTAAAAATCCAGAC |
| | | TCTTTCGATACCCAGGACCAAGCCACAGCAGGTCCTCCATCCCAACAGCCATGCC |
| | | CGCATTAGCTCTTAGACCCACAGACTGGTTTTGCAACGTTTACACCGACTAGCCA |
| | | GGAAGTACTTCCACCTCGGGCACATTTTGGGAAGTTGCATTCCTTTGTCTTCAAA |
| | | CTGTGAAGCATTTACAGAAACGCATCCAGCAAGAATATTGTCCCTTTGAGCAGA |
| | | AATTTATCTTTCAAAGAGGTATATTTGAAAAAAAAAAAAAGTATATGTGAGGAT |
| | | TTTTATTGATTGGGGATCTTGGAGTTTTTCATTGTCGCTATTGATTTTTACTTCAAT |
| | | GGGCTCTTCCAACAAGGAAGAAGCTTGCTGGTAGCACTTGCTACCCTGAGTTCAT |
| | | CCAGGCCCAACTGTGAGCAAGGAGCACAAGCCACAAGTCTTCCAGAGGATGCTT |
| | | GATTCCAGTGGTTCTGCTTCAAGGCTTCCACTGCAAAACACTAAAGATCCAAGAA |
| | | GGCCTTCATGGCCCCAGCAGGCCGGATCGGTACTGTATCAAGTCATGGCAGGTA |
| | | CAGTAGGATAAGCCACTCTGTCCCTTCCTGGGCAAAGAAGAAACGGAGGGGATG |
| | | GAATTCTTCCTTAGACTTACTTTTGTAAAAATGTCCCCACGGTACTTACTCCCCAC |
| | | TGATGGACCAGTGGTTTCCAGTCATGAGCGTTAGACTGACTTGTTTGTCTTCCATT |
| | | CCATTGTTTTGAAACTCAGTATGCTGCCCCTGTCTTGCTGTCATGAAATCAGCAA |
| | | GAGAGGATGACACATCAAATAATAACTCGGATTCCAGCCCACATTGGATTCATC |
| | | AGCATTTGGACCAATAGCCCACAGCTGAGAATGTGGAATACCTAAGGATAGCAC |
| | | CGCTTTTGTTCTCGCAAAAACGTATCTCCTAATTTGAGGCTCAGATGAAATGCAT |
| | | CAGGTCCTTTGGGGCATAGATCAGAAGACTACAAAAATGAAGCTGCTCTGAAAT |
| | | CTCCTTTAGCCATCACCCCAACCCCCCAAAATTAGTTTGTGTTACTTATGGAAGA |
| | | TAGTTTTCTCCTTTTACTTCACTTCAAAAGCTTTTTACTCAAAGAGTATATGTTCC |
| | | CTCCAGGTCAGCTGCCCCCAAACCCCCTCCTTACGCTTTGTCACACAAAAAGTGT |
| | | CTCTGCCTTGAGTCATCTATTCAAGCACTTACAGCTCTGGCCACAACAGGGCATT |
| | | TTACAGGTGCGAATGACAGTAGCATTATGAGTAGTGTGGAATTCAGGTAGTAAA |
| | | TATGAAACTAGGGTTTGAAATTGATAATGCTTTCACAACATTTGCAGATGTTTTA |
| | | GAAGGAAAAAAGTTCCTTCCTAAAATAATTTCTCTACAATTGGAAGATTGGAAG |
| | | ATTCAGCTAGTTAGGAGCCCACCTTTTTTCCTAATCTGTGTGTGCCCTGTAACCTG |
| | | ACTGGTTAACAGCAGTCCTTTGTAAACAGTGTTTTAAACTCTCCTAGTCAATATC |
| | | CACCCCATCCAATTTATCAAGGAAGAAATGGTTCAGAAAATATTTTCAGCCTACA |
| | | GTTATGTTCAGTCACACACACATACAAAATGTTCCTTTTGCTTTTAAAGTAATTTT |
| | | TGACTCCCAGATCAGTCAGAGCCCCTACAGCATTGTTAAGAAAGTATTTGATTTT |
| | | TGTCTCAATGAAAATAAAACTATATTCATTTCCACTCTATTATGCTCTCAAATACC |
| | | CCTAAGCATCTATACTAGCCTGGTATGGGTATGAAAGATACAAAGATAAATAAA |
| | | ACATAGTCCCTGATTCTAAGAAATTCACAATTTAGCAAAGGAAATGGACTCATA |
| | | GATGCTAACCTTAAAACAACGTGACAAATGCCAGACAGGACCCATCAGCCAGGC |
| | | ACTGTGAGAGCACAGAGCAGGGAGGTTGGGTCCTGCCTGAGGAGACCTGGAAGG |
| | | GAGGCCTCACAGGAGGATGACCAGGTCTCAGTCAGCGGGGAGGTGGAAAGTGC |
| | | AGGTGCATCAGGGGCACCCTGACCGAGGAAACAGCTGCCAGAGGCCTCCACTGC |
| | | TAAAGTCCACATAAGGCTGAGGTCAGTCACCCTAAACAACCTGCTCCCTCTAAGC |
| | | CAGGGGATGAGCTTGGAGCATCCCACAAGTTCCCTAAAAGTTGCAGCCCCCAGG |
| | | GGGATTTTGAGCTATCATCTCTGCACATGCTTAGTGAGAAGACTACACAACATTT |
| | | CTAAGAATCTGAGATTTTATATTGTCAGTTAACCACTTTCATTATTCATTCACCTC |
| | | AGGACATGCAGAAATATTTCAGTCAGAACTGGGAAACAGAAGGACCTACATTCT |
| | | GCTGTCACTTATGTGTCAAGAAGCAGATGATCGATGAGGCAGGTCAGTTGTAAG |
| | | TGAGTCACATTGTAGCATTAAATTCTAGTATTTTTGTAGTTTGAAACAGTAACTTA |
| | | ATAAAAGAGCAAAAGCTATTCTAGCTTTCTTCTTCATATTTTAATTTTCCACCATA |
| | | AAGTTTAGTTGCTAAATTCTATTAATTTTAAGATTGTGCTTCCCAAAATAGTTCTC |
| | | ACTTCATCTGTCCAGGGAGGCACAGTTCTGTCTGGTAGAAGCCGCAAAGCCCTTA |
| | | GCCTCTTCACGGATCTGGCGACTGTGATGGGCAGGTCAGGAGAGGAGCTGCCCA |
| | | AAGTCCCATGATTTTCACCTAACAGCCCTGATCAGTCAGTACTCAAAGCTTGGAC |
| | | TCCATCCCTGAAGGTCTTCCTGATTGATAGCCTGGCCTTAATACCCTACAGAAAG |
| | | CCTGTCCATTGGCTGTTTCTTCCTCAGTCAGTTCCTGGAAGACCTTACCCCATGAC |
| | | CCCAGCTTCAGATGTGGTCTTTGGAAACAGAGGTCGAAGGAAAGTAAGGAGCTG |
| | | AGAGCTCACATTCATAGGTGCCGCCAGCCTTCGTGCATCTTCTTGCATCATCTCTA |
| | | AGGAGCTCCTCTAATTACACCATGCCCGTCACCCCATGAGGGATCAGAGAAGGG |
| | | ATGAGTCTTCTAAACTCTATATTCGCTGTGAGTCCAGGTTGTAAGGGGGAGCACT |
| | | GTGGATGCATCCTATTGCACTCCAGCTGATGACACCAAAGCTTAGGTGTTTGCTG |
| | | AAAGTTCTTGATGTTGTGACTTACCACCCCTGCCTCACAACTGCAGACATAAGGG |
| | | GACTATGGATTGCTTAGCAGGAAAGGCACTGGTTCTCAAGGGCGGCTGCCCTTG |
| | | GGAATCTTCTGGTCCCAACCAGAAAGACTGTGGCTTGATTTTCTCAGGTGCAGCC |
| | | CAGCCGTAGGGCCTTTTCAGAGCACCCCCTGGTTATTGCAACATTCATCAAAGTT |
| | | TCTAGAACCTCTGGCCTAAAGGAAGGGCCTGGTGGGATCTACTTGGCACTCGCTG |
| | | GGGGGCCACCCCCCAGTGCCACTCTCACTAGGCCTCTGATTGCACTTGTGTAGGA |
| | | TGAAGCTGGTGGGTGATGGGAACTCAGCACCTCCCCTCAGGCAGAAAAGAATCA |
| | | TCTGTGGAGCTTCAAAAGAAGGGGCCTGGAGTCTCTGCAGACCAATTCAACCCA |
| | | AATCTCGGGGGCTCTTTCATGATTCTAATGGGCAACCAGGGTTGAAACCCTTATT |
| | | TCTAGGGTCTTCAGTTGTACAAGACTGTGGGTCTGTACCAGAGCCCCCGTCAGAG |
| | | TAGAATAAAAGGCTGGGTAGGGTAGAGATTCCCATGTGCAGTGGAGAGAACAAT |
| | | CTGCAGTCACTGATAAGCCTGAGACTTGGCTCATTTCAAAAGCGTTCAATTCATC |
| | | CTCACCAGCAGTTCAGCTGGAAAGGGGCAAATACCCCCACCTGAGCTTTGAAAA |
| | | CGCCCTGGGACCCTCTGCATTCTCTAAGTAAGTTATAGAAACCAGTCTCTTCCCT |
| | | CCTTTGTGAGTGAGCTGCTATTCCACGTAGGCAACACCTGTTGAAATTGCCCTCA |
| | | ATGTCTACTCTGCATTTCTTTCTTGTGATAAGCACACACTTTTATTGCAACATAAT |
| | | GATCTGCTCACATTTCCTTGCCTGGGGGCTGTAAAACCTTACAGAACAGAAATCC |
| | | TTGCCTCTTTCACCAGCCACACCTGCCATACCAGGGGTACAGCTTTGTACTATTG |
| | | AAGACACAGACAGGATTTTTAAATGTAAATCTATTTTTGTAACTTTGTTGCGGGA |
| | | TATAGTTCTCTTTATGTAGCACTGAACTTTGTACAATATATTTTTAGAAACTCATT |
| | | TTTCTACTAAAACAAACACAGTTTACTTTAGAGAGACTGCAATAGAATCAAAATT |
| | | TGAAACTGAAATCTTTGTTTAAAAGGGTTAAGTTGAGGCAAGAGGAAAGCCCTT |
| | | TCTCTCTCTTATAAAAAGGCACAACCTCATTGGGGAGCTAAGCTAGGTCATTGTC |
| | | ATGGTGAAGAAGAGAAGCATCGTTTTTATATTTAGGAAATTTTAAAAGATGATG |
| | | GAAAGCACATTTAGCTTGGTCTGAGGCAGGTTCTGTTGGGGCAGTGTTAATGGAA |
| | | AGGGCTCACTGTTGTTACTACTAGAAAAATCCAGTTGCATGCCATACTCTCATCA |
| | | TCTGCCAGTGTAACCCTGTACATGTAAGAAAAGCAATAACATAGCACTTTGTTGG |
| | | TTTATATATATAATGTGACTTCAATGCAAATTTTATTTTTATATTTACAATTGATA |
| | | TGCATTTACCAGTATAAACTAGACATGTCTGGAGAGCCTAATAATGTTCAGCACA |
| | | CTTTGGTTAGTTCACCAACAGTCTTACCAAGCCTGGGCCCAGCCACCCTAGAGAA |
| | | GTTATTCAGCCCTGGCTGCAGTGACATCACCTGAGGAGCTTTTAAAAGCTTGAAG |
| | | CCCAGCTACACCTCAGACCGATTAAACGCAAATCTCTGGGGCTGAAACCCAAGC |
| | | ATTCGTAGTTTTTAAAGCTCCTGAGGTCATTCCAATGTGCGGCCAAAGTTGAGAA |
| | | CTACTGGCCTAGGGATTAGCCACAAGGACATGGACTTGGAGGCAAATTCTGCAG |
| | | GTGTATGTGATTCTCAGGCCTAGAGAGCTAAGACACAAAGACCTCCACATCTGTC |
| | | GCTGAGAGTCAAGAACCTGAACAGAGTTTCCATGAAGGTTCTCCAAGCACTAGA |
| | | AGGGAGAGTGTCTAAACAATGGTTGAAAAGCAAAGGAAATATAAAACAGACAC |
| | | CTCTTTCCATTTCCTAAGGTTTCTCTCTTTATTAAGGGTGGACTAGTAATAAAATA |
| | | TAATATTCTTGCTGCTTATGCAGCTGACATTGTTGCCCTCCCTAAAGCAACCAAG |
| | | TAGCCTTTATTTCCCACAGTGAAAGAAAACGCTGGCCTATCAGTTACATTACAAA |
| | | AGGCAGATTTCAAGAGGATTGAGTAAGTAGTTGGATGGCTTTCATAAAAACAAG |
| | | AATTCAAGAAGAGGATTCATGCTTTAAGAAACATTTGTTATACATTCCTCACAAA |
| | | TTATACCTGGGATAAAAACTATGTAGCAGGCAGTGTGTTTTCCTTCCATGTCTCTC |
| | | TGCACTACCTGCAGTGTGTCCTCTGAGGCTGCAAGTCTGTCCTATCTGAATTCCC |
| | | AGCAGAAGCACTAAGAAGCTCCACCCTATCACCTAGCAGATAAAACTATGGGGA |
| | | AAACTTAAATCTGTGCATACATTTCTGGATGCATTTACTTATCTTTAAAAAAAAA |
| | | GGAATCCTATGACCTGATTTGGCCACAAAAATAATCTTGCTGTACAATACAATCT |
| | | CTTGGAAATTAAGAGATCCTATGGATTTGATGACTGGTATTAGAGGTGACAATGT |
| | | AACCGATTAACAACAGACAGCAATAACTTCGTTTTAGAAACATTCAAGCAATAG |
| | | CTTTATAGCTTCAACATATGGTACGTTTTAACCTTGAAAGTTTTGCAATGATGAA |
| | | AGCAGTATTTGTACAAATGAAAAGCAGAATTCTCTTTTATATGGTTTATACTGTT |
| | | GATCAGAAATGTTGATTGTGCATTGAGTATTAAAAAATTAGATGTATATTATTCA |
| | | TTGTTCTTTACTCCTGAGTACCTTATAATAATAATAATGTATTCTTTGTTAACAA |
| SEQ | KRAS | CTAGGCGGCGGCCGCGGCGGCGGAGGCAGCAGCGGCGGCGGCAGTGGCGGCGG |
| ID | | CGAAGGTGGCGGCGGCTCGGCCAGTACTCCCGGCCCCCGCCATTTCGGACTGGG |
| NO: | | AGCGAGCGCGGCGCAGGCACTGAAGGCGGCGGCGGGGCCAGAGGCTCAGCGGC |
| 423 | | TCCCAGGCCTGCTGAAAATGACTGAATATAAACTTGTGGTAGTTGGAGCTGGTGG |
| | | CGTAGGCAAGAGTGCCTTGACGATACAGCTAATTCAGAATCATTTTGTGGACGA |
| | | ATATGATCCAACAATAGAGGATTCCTACAGGAAGCAAGTAGTAATTGATGGAGA |
| | | AACCTGTCTCTTGGATATTCTCGACACAGCAGGTCAAGAGGAGTACAGTGCAAT |
| | | GAGGGACCAGTACATGAGGACTGGGGAGGGCTTTCTTTGTGTATTTGCCATAAAT |
| | | AATACTAAATCATTTGAAGATATTCACCATTATAGAGAACAAATTAAAAGAGTT |
| | | AAGGACTCTGAAGATGTACCTATGGTCCTAGTAGGAAATAAATGTGATTTGCCTT |
| | | CTAGAACAGTAGACACAAAACAGGCTCAGGACTTAGCAAGAAGTTATGGAATTC |
| | | CTTTTATTGAAACATCAGCAAAGACAAGACAGGGTGTTGATGATGCCTTCTATAC |
| | | ATTAGTTCGAGAAATTCGAAAACATAAAGAAAAGATGAGCAAAGATGGTAAAA |
| | | AGAAGAAAAAGAAGTCAAAGACAAAGTGTGTAATTATGTAAATACAATTTGTAC |
| | | TTTTTTCTTAAGGCATACTAGTACAAGTGGTAATTTTTGTACATTACACTAAATTA |
| | | TTAGCATTTGTTTTAGCATTACCTAATTTTTTTCCTGCTCCATGCAGACTGTTAGC |
| | | TTTTACCTTAAATGCTTATTTTAAAATGACAGTGGAAGTTTTTTTTTCCTCTAAGT |
| | | GCCAGTATTCCCAGAGTTTTGGTTTTTGAACTAGCAATGCCTGTGAAAAAGAAAC |
| | | TGAATACCTAAGATTTCTGTCTTGGGGCTTTTGGTGCATGCAGTTGATTACTTCTT |
| | | ATTTTTCTTACCAATTGTGAATGTTGGTGTGAAACAAATTAATGAAGCTTTTGAA |
| | | TCATCCCTATTCTGTGTTTTATCTAGTCACATAAATGGATTAATTACTAATTTCAG |
| | | TTGAGACCTTCTAATTGGTTTTTACTGAAACATTGAGGGAACACAAATTTATGGG |
| | | CTTCCTGATGATGATTCTTCTAGGCATCATGTCCTATAGTTTGTCATCCCTGATGA |
| | | ATGTAAAGTTACACTGTTCACAAAGGTTTTGTCTCCTTTCCACTGCTATTAGTCAT |
| | | GGTCACTCTCCCCAAAATATTATATTTTTTCTATAAAAAGAAAAAAATGGAAAAA |
| | | AATTACAAGGCAATGGAAACTATTATAAGGCCATTTCCTTTTCACATTAGATAAA |
| | | TTACTATAAAGACTCCTAATAGCTTTTCCTGTTAAGGCAGACCCAGTATGAAATG |
| | | GGGATTATTATAGCAACCATTTTGGGGCTATATTTACATGCTACTAAATTTTTATA |
| | | ATAATTGAAAAGATTTTAACAAGTATAAAAAATTCTCATAGGAATTAAATGTAGT |
| | | CTCCCTGTGTCAGACTGCTCTTTCATAGTATAACTTTAAATCTTTTCTTCAACTTG |
| | | AGTCTTTGAAGATAGTTTTAATTCTGCTTGTGACATTAAAAGATTATTTGGGCCA |
| | | GTTATAGCTTATTAGGTGTTGAAGAGACCAAGGTTGCAAGGCCAGGCCCTGTGTG |
| | | AACCTTTGAGCTTTCATAGAGAGTTTCACAGCATGGACTGTGTCCCCACGGTCAT |
| | | CCAGTGTTGTCATGCATTGGTTAGTCAAAATGGGGAGGGACTAGGGCAGTTTGG |
| | | ATAGCTCAACAAGATACAATCTCACTCTGTGGTGGTCCTGCTGACAAATCAAGAG |
| | | CATTGCTTTTGTTTCTTAAGAAAACAAACTCTTTTTTAAAAATTACTTTTAAATAT |
| | | TAACTCAAAAGTTGAGATTTTGGGGTGGTGGTGTGCCAAGACATTAATTTTTTTT |
| | | TTAAACAATGAAGTGAAAAAGTTTTACAATCTCTAGGTTTGGCTAGTTCTCTTAA |
| | | CACTGGTTAAATTAACATTGCATAAACACTTTTCAAGTCTGATCCATATTTAATA |
| | | ATGCTTTAAAATAAAAATAAAAACAATCCTTTTGATAAATTTAAAATGTTACTTA |
| | | TTTTAAAATAAATGAAGTGAGATGGCATGGTGAGGTGAAAGTATCACTGGACTA |
| | | GGAAGAAGGTGACTTAGGTTCTAGATAGGTGTCTTTTAGGACTCTGATTTTGAGG |
| | | ACATCACTTACTATCCATTTCTTCATGTTAAAAGAAGTCATCTCAAACTCTTAGTT |
| | | TTTTTTTTTTACAACTATGTAATTTATATTCCATTTACATAAGGATACACTTATTTG |
| | | TCAAGCTCAGCACAATCTGTAAATTTTTAACCTATGTTACACCATCTTCAGTGCC |
| | | AGTCTTGGGCAAAATTGTGCAAGAGGTGAAGTTTATATTTGAATATCCATTCTCG |
| | | TTTTAGGACTCTTCTTCCATATTAGTGTCATCTTGCCTCCCTACCTTCCACATGCC |
| | | CCATGACTTGATGCAGTTTTAATACTTGTAATTCCCCTAACCATAAGATTTACTGC |
| | | TGCTGTGGATATCTCCATGAAGTTTTCCCACTGAGTCACATCAGAAATGCCCTAC |
| | | ATCTTATTTCCTCAGGGCTCAAGAGAATCTGACAGATACCATAAAGGGATTTGAC |
| | | CTAATCACTAATTTTCAGGTGGTGGCTGATGCTTTGAACATCTCTTTGCTGCCCAA |
| | | TCCATTAGCGACAGTAGGATTTTTCAAACCTGGTATGAATAGACAGAACCCTATC |
| | | CAGTGGAAGGAGAATTTAATAAAGATAGTGCTGAAAGAATTCCTTAGGTAATCT |
| | | ATAACTAGGACTACTCCTGGTAACAGTAATACATTCCATTGTTTTAGTAACCAGA |
| | | AATCTTCATGCAATGAAAAATACTTTAATTCATGAAGCTTACTTTTTTTTTTTGGT |
| | | GTCAGAGTCTCGCTCTTGTCACCCAGGCTGGAATGCAGTGGCGCCATCTCAGCTC |
| | | ACTGCAACCTCCATCTCCCAGGTTCAAGCGATTCTCGTGCCTCGGCCTCCTGAGT |
| | | AGCTGGGATTACAGGCGTGTGCCACTACACTCAACTAATTTTTGTATTTTTAGGA |
| | | GAGACGGGGTTTCACCCTGTTGGCCAGGCTGGTCTCGAACTCCTGACCTCAAGTG |
| | | ATTCACCCACCTTGGCCTCATAAACCTGTTTTGCAGAACTCATTTATTCAGCAAAT |
| | | ATTTATTGAGTGCCTACCAGATGCCAGTCACCACACAAGGCACTGGGTATATGGT |
| | | ATCCCCAAACAAGAGACATAATCCCGGTCCTTAGGTAGTGCTAGTGTGGTCTGTA |
| | | ATATCTTACTAAGGCCTTTGGTATACGACCCAGAGATAACACGATGCGTATTTTA |
| | | GTTTTGCAAAGAAGGGGTTTGGTCTCTGTGCCAGCTCTATAATTGTTTTGCTACG |
| | | ATTCCACTGAAACTCTTCGATCAAGCTACTTTATGTAAATCACTTCATTGTTTTAA |
| | | AGGAATAAACTTGATTATATTGTTTTTTTATTTGGCATAACTGTGATTCTTTTAGG |
| | | ACAATTACTGTACACATTAAGGTGTATGTCAGATATTCATATTGACCCAAATGTG |
| | | TAATATTCCAGTTTTCTCTGCATAAGTAATTAAAATATACTTAAAAATTAATAGTT |
| | | TTATCTGGGTACAAATAAACAGGTGCCTGAACTAGTTCACAGACAAGGAAACTT |
| | | CTATGTAAAAATCACTATGATTTCTGAATTGCTATGTGAAACTACAGATCTTTGG |
| | | AACACTGTTTAGGTAGGGTGTTAAGACTTACACAGTACCTCGTTTCTACACAGAG |
| | | AAAGAAATGGCCATACTTCAGGAACTGCAGTGCTTATGAGGGGATATTTAGGCC |
| | | TCTTGAATTTTTGATGTAGATGGGCATTTTTTTAAGGTAGTGGTTAATTACCTTTA |
| | | TGTGAACTTTGAATGGTTTAACAAAAGATTTGTTTTTGTAGAGATTTTAAAGGGG |
| | | GAGAATTCTAGAAATAAATGTTACCTAATTATTACAGCCTTAAAGACAAAAATCC |
| | | TTGTTGAAGTTTTTTTAAAAAAAGCTAAATTACATAGACTTAGGCATTAACATGT |
| | | TTGTGGAAGAATATAGCAGACGTATATTGTATCATTTGAGTGAATGTTCCCAAGT |
| | | AGGCATTCTAGGCTCTATTTAACTGAGTCACACTGCATAGGAATTTAGAACCTAA |
| | | CTTTTATAGGTTATCAAAACTGTTGTCACCATTGCACAATTTTGTCCTAATATATA |
| | | CATAGAAACTTTGTGGGGCATGTTAAGTTACAGTTTGCACAAGTTCATCTCATTT |
| | | GTATTCCATTGATTTTTTTTTTCTTCTAAACATTTTTTCTTCAAACAGTATATAACT |
| | | TTTTTTAGGGGATTTTTTTTTAGACAGCAAAAACTATCTGAAGATTTCCATTTGTC |
| | | AAAAAGTAATGATTTCTTGATAATTGTGTAGTAATGTTTTTTAGAACCCAGCAGT |
| | | TACCTTAAAGCTGAATTTATATTTAGTAACTTCTGTGTTAATACTGGATAGCATG |
| | | AATTCTGCATTGAGAAACTGAATAGCTGTCATAAAATGAAACTTTCTTTCTAAAG |
| | | AAAGATACTCACATGAGTTCTTGAAGAATAGTCATAACTAGATTAAGATCTGTGT |
| | | TTTAGTTTAATAGTTTGAAGTGCCTGTTTGGGATAATGATAGGTAATTTAGATGA |
| | | ATTTAGGGGAAAAAAAAGTTATCTGCAGATATGTTGAGGGCCCATCTCTCCCCCC |
| | | ACACCCCCACAGAGCTAACTGGGTTACAGTGTTTTATCCGAAAGTTTCCAATTCC |
| | | ACTGTCTTGTGTTTTCATGTTGAAAATACTTTTGCATTTTTCCTTTGAGTGCCAATT |
| | | TCTTACTAGTACTATTTCTTAATGTAACATGTTTACCTGGAATGTATTTTAACTAT |
| | | TTTTGTATAGTGTAAACTGAAACATGCACATTTTGTACATTGTGCTTTCTTTTGTG |
| | | GGACATATGCAGTGTGATCCAGTTGTTTTCCATCATTTGGTTGCGCTGACCTAGG |
| | | AATGTTGGTCATATCAAACATTAAAAATGACCACTCTTTTAATTGAAATTAACTT |
| | | TTAAATGTTTATAGGAGTATGTGCTGTGAAGTGATCTAAAATTTGTAATATTTTTG |
| | | TCATGAACTGTACTACTCCTAATTATTGTAATGTAATAAAAATAGTTACAGTGAC |
| SEQ | KRAS | CTAGGCGGCGGCCGCGGCGGCGGAGGCAGCAGCGGCGGCGGCAGTGGCGGCGG |
| ID | | CGAAGGTGGCGGCGGCTCGGCCAGTACTCCCGGCCCCCGCCATTTCGGACTGGG |
| NO: | | AGCGAGCGCGGCGCAGGCACTGAAGGCGGCGGCGGGGCCAGAGGCTCAGCGGC |
| 424 | | TCCCAGGCCTGCTGAAAATGACTGAATATAAACTTGTGGTAGTTGGAGCTGGTGG |
| | | CGTAGGCAAGAGTGCCTTGACGATACAGCTAATTCAGAATCATTTTGTGGACGA |
| | | ATATGATCCAACAATAGAGGATTCCTACAGGAAGCAAGTAGTAATTGATGGAGA |
| | | AACCTGTCTCTTGGATATTCTCGACACAGCAGGTCAAGAGGAGTACAGTGCAAT |
| | | GAGGGACCAGTACATGAGGACTGGGGAGGGCTTTCTTTGTGTATTTGCCATAAAT |
| | | AATACTAAATCATTTGAAGATATTCACCATTATAGAGAACAAATTAAAAGAGTT |
| | | AAGGACTCTGAAGATGTACCTATGGTCCTAGTAGGAAATAAATGTGATTTGCCTT |
| | | CTAGAACAGTAGACACAAAACAGGCTCAGGACTTAGCAAGAAGTTATGGAATTC |
| | | CTTTTATTGAAACATCAGCAAAGACAAGACAGAGAGTGGAGGATGCTTTTTATA |
| | | CATTGGTGAGAGAGATCCGACAATACAGATTGAAAAAAATCAGCAAAGAAGAA |
| | | AAGACTCCTGGCTGTGTGAAAATTAAAAAATGCATTATAATGTAATCTGGGTGTT |
| | | GATGATGCCTTCTATACATTAGTTCGAGAAATTCGAAAACATAAAGAAAAGATG |
| | | AGCAAAGATGGTAAAAAGAAGAAAAAGAAGTCAAAGACAAAGTGTGTAATTAT |
| | | GTAAATACAATTTGTACTTTTTTCTTAAGGCATACTAGTACAAGTGGTAATTTTTG |
| | | TACATTACACTAAATTATTAGCATTTGTTTTAGCATTACCTAATTTTTTTCCTGCTC |
| | | CATGCAGACTGTTAGCTTTTACCTTAAATGCTTATTTTAAAATGACAGTGGAAGT |
| | | TTTTTTTTCCTCTAAGTGCCAGTATTCCCAGAGTTTTGGTTTTTGAACTAGCAATG |
| | | CCTGTGAAAAAGAAACTGAATACCTAAGATTTCTGTCTTGGGGCTTTTGGTGCAT |
| | | GCAGTTGATTACTTCTTATTTTTCTTACCAATTGTGAATGTTGGTGTGAAACAAAT |
| | | TAATGAAGCTTTTGAATCATCCCTATTCTGTGTTTTATCTAGTCACATAAATGGAT |
| | | TAATTACTAATTTCAGTTGAGACCTTCTAATTGGTTTTTACTGAAACATTGAGGG |
| | | AACACAAATTTATGGGCTTCCTGATGATGATTCTTCTAGGCATCATGTCCTATAG |
| | | TTTGTCATCCCTGATGAATGTAAAGTTACACTGTTCACAAAGGTTTTGTCTCCTTT |
| | | CCACTGCTATTAGTCATGGTCACTCTCCCCAAAATATTATATTTTTTCTATAAAAA |
| | | GAAAAAAATGGAAAAAAATTACAAGGCAATGGAAACTATTATAAGGCCATTTCC |
| | | TTTTCACATTAGATAAATTACTATAAAGACTCCTAATAGCTTTTCCTGTTAAGGCA |
| | | GACCCAGTATGAAATGGGGATTATTATAGCAACCATTTTGGGGCTATATTTACAT |
| | | GCTACTAAATTTTTATAATAATTGAAAAGATTTTAACAAGTATAAAAAATTCTCA |
| | | TAGGAATTAAATGTAGTCTCCCTGTGTCAGACTGCTCTTTCATAGTATAACTTTAA |
| | | ATCTTTTCTTCAACTTGAGTCTTTGAAGATAGTTTTAATTCTGCTTGTGACATTAA |
| | | AAGATTATTTGGGCCAGTTATAGCTTATTAGGTGTTGAAGAGACCAAGGTTGCAA |
| | | GGCCAGGCCCTGTGTGAACCTTTGAGCTTTCATAGAGAGTTTCACAGCATGGACT |
| | | GTGTCCCCACGGTCATCCAGTGTTGTCATGCATTGGTTAGTCAAAATGGGGAGGG |
| | | ACTAGGGCAGTTTGGATAGCTCAACAAGATACAATCTCACTCTGTGGTGGTCCTG |
| | | CTGACAAATCAAGAGCATTGCTTTTGTTTCTTAAGAAAACAAACTCTTTTTTAAA |
| | | AATTACTTTTAAATATTAACTCAAAAGTTGAGATTTTGGGGTGGTGGTGTGCCAA |
| | | GACATTAATTTTTTTTTTAAACAATGAAGTGAAAAAGTTTTACAATCTCTAGGTTT |
| | | GGCTAGTTCTCTTAACACTGGTTAAATTAACATTGCATAAACACTTTTCAAGTCT |
| | | GATCCATATTTAATAATGCTTTAAAATAAAAATAAAAACAATCCTTTTGATAAAT |
| | | TTAAAATGTTACTTATTTTAAAATAAATGAAGTGAGATGGCATGGTGAGGTGAA |
| | | AGTATCACTGGACTAGGAAGAAGGTGACTTAGGTTCTAGATAGGTGTCTTTTAGG |
| | | ACTCTGATTTTGAGGACATCACTTACTATCCATTTCTTCATGTTAAAAGAAGTCAT |
| | | CTCAAACTCTTAGTTTTTTTTTTTTACAACTATGTAATTTATATTCCATTTACATAA |
| | | GGATACACTTATTTGTCAAGCTCAGCACAATCTGTAAATTTTTAACCTATGTTAC |
| | | ACCATCTTCAGTGCCAGTCTTGGGCAAAATTGTGCAAGAGGTGAAGTTTATATTT |
| | | GAATATCCATTCTCGTTTTAGGACTCTTCTTCCATATTAGTGTCATCTTGCCTCCC |
| | | TACCTTCCACATGCCCCATGACTTGATGCAGTTTTAATACTTGTAATTCCCCTAAC |
| | | CATAAGATTTACTGCTGCTGTGGATATCTCCATGAAGTTTTCCCACTGAGTCACA |
| | | TCAGAAATGCCCTACATCTTATTTCCTCAGGGCTCAAGAGAATCTGACAGATACC |
| | | ATAAAGGGATTTGACCTAATCACTAATTTTCAGGTGGTGGCTGATGCTTTGAACA |
| | | TCTCTTTGCTGCCCAATCCATTAGCGACAGTAGGATTTTTCAAACCTGGTATGAA |
| | | TAGACAGAACCCTATCCAGTGGAAGGAGAATTTAATAAAGATAGTGCTGAAAGA |
| | | ATTCCTTAGGTAATCTATAACTAGGACTACTCCTGGTAACAGTAATACATTCCAT |
| | | TGTTTTAGTAACCAGAAATCTTCATGCAATGAAAAATACTTTAATTCATGAAGCT |
| | | TACTTTTTTTTTTTGGTGTCAGAGTCTCGCTCTTGTCACCCAGGCTGGAATGCAGT |
| | | GGCGCCATCTCAGCTCACTGCAACCTCCATCTCCCAGGTTCAAGCGATTCTCGTG |
| | | CCTCGGCCTCCTGAGTAGCTGGGATTACAGGCGTGTGCCACTACACTCAACTAAT |
| | | TTTTGTATTTTTAGGAGAGACGGGGTTTCACCCTGTTGGCCAGGCTGGTCTCGAA |
| | | CTCCTGACCTCAAGTGATTCACCCACCTTGGCCTCATAAACCTGTTTTGCAGAAC |
| | | TCATTTATTCAGCAAATATTTATTGAGTGCCTACCAGATGCCAGTCACCACACAA |
| | | GGCACTGGGTATATGGTATCCCCAAACAAGAGACATAATCCCGGTCCTTAGGTA |
| | | GTGCTAGTGTGGTCTGTAATATCTTACTAAGGCCTTTGGTATACGACCCAGAGAT |
| | | AACACGATGCGTATTTTAGTTTTGCAAAGAAGGGGTTTGGTCTCTGTGCCAGCTC |
| | | TATAATTGTTTTGCTACGATTCCACTGAAACTCTTCGATCAAGCTACTTTATGTAA |
| | | ATCACTTCATTGTTTTAAAGGAATAAACTTGATTATATTGTTTTTTTATTTGGCAT |
| | | AACTGTGATTCTTTTAGGACAATTACTGTACACATTAAGGTGTATGTCAGATATT |
| | | CATATTGACCCAAATGTGTAATATTCCAGTTTTCTCTGCATAAGTAATTAAAATA |
| | | TACTTAAAAATTAATAGTTTTATCTGGGTACAAATAAACAGGTGCCTGAACTAGT |
| | | TCACAGACAAGGAAACTTCTATGTAAAAATCACTATGATTTCTGAATTGCTATGT |
| | | GAAACTACAGATCTTTGGAACACTGTTTAGGTAGGGTGTTAAGACTTACACAGTA |
| | | CCTCGTTTCTACACAGAGAAAGAAATGGCCATACTTCAGGAACTGCAGTGCTTAT |
| | | GAGGGGATATTTAGGCCTCTTGAATTTTTGATGTAGATGGGCATTTTTTTAAGGT |
| | | AGTGGTTAATTACCTTTATGTGAACTTTGAATGGTTTAACAAAAGATTTGTTTTTG |
| | | TAGAGATTTTAAAGGGGGAGAATTCTAGAAATAAATGTTACCTAATTATTACAGC |
| | | CTTAAAGACAAAAATCCTTGTTGAAGTTTTTTTAAAAAAAGCTAAATTACATAGA |
| | | CTTAGGCATTAACATGTTTGTGGAAGAATATAGCAGACGTATATTGTATCATTTG |
| | | AGTGAATGTTCCCAAGTAGGCATTCTAGGCTCTATTTAACTGAGTCACACTGCAT |
| | | AGGAATTTAGAACCTAACTTTTATAGGTTATCAAAACTGTTGTCACCATTGCACA |
| | | ATTTTGTCCTAATATATACATAGAAACTTTGTGGGGCATGTTAAGTTACAGTTTG |
| | | CACAAGTTCATCTCATTTGTATTCCATTGATTTTTTTTTTCTTCTAAACATTTTTTC |
| | | TTCAAACAGTATATAACTTTTTTTAGGGGATTTTTTTTTAGACAGCAAAAACTATC |
| | | TGAAGATTTCCATTTGTCAAAAAGTAATGATTTCTTGATAATTGTGTAGTAATGTT |
| | | TTTTAGAACCCAGCAGTTACCTTAAAGCTGAATTTATATTTAGTAACTTCTGTGTT |
| | | AATACTGGATAGCATGAATTCTGCATTGAGAAACTGAATAGCTGTCATAAAATG |
| | | AAACTTTCTTTCTAAAGAAAGATACTCACATGAGTTCTTGAAGAATAGTCATAAC |
| | | TAGATTAAGATCTGTGTTTTAGTTTAATAGTTTGAAGTGCCTGTTTGGGATAATG |
| | | ATAGGTAATTTAGATGAATTTAGGGGAAAAAAAAGTTATCTGCAGATATGTTGA |
| | | GGGCCCATCTCTCCCCCCACACCCCCACAGAGCTAACTGGGTTACAGTGTTTTAT |
| | | CCGAAAGTTTCCAATTCCACTGTCTTGTGTTTTCATGTTGAAAATACTTTTGCATT |
| | | TTTCCTTTGAGTGCCAATTTCTTACTAGTACTATTTCTTAATGTAACATGTTTACCT |
| | | GGAATGTATTTTAACTATTTTTGTATAGTGTAAACTGAAACATGCACATTTTGTAC |
| | | ATTGTGCTTTCTTTTGTGGGACATATGCAGTGTGATCCAGTTGTTTTCCATCATTT |
| | | GGTTGCGCTGACCTAGGAATGTTGGTCATATCAAACATTAAAAATGACCACTCTT |
| | | TTAATTGAAATTAACTTTTAAATGTTTATAGGAGTATGTGCTGTGAAGTGATCTA |
| | | AAATTTGTAATATTTTTGTCATGAACTGTACTACTCCTAATTATTGTAATGTAATA |
| | | AAAATAGTTACAGTGAC |
| SEQ | KRAS | CTAGGCGGCGGCCGCGGCGGCGGAGGCAGCAGCGGCGGCGGCAGTGGCGGCGG |
| ID | | CGAAGGTGGCGGCGGCTCGGCCAGTACTCCCGGCCCCCGCCATTTCGGACTGGG |
| NO: | | AGCGAGCGCGGCGCAGGCACTGAAGGCGGCGGCGGGGCCAGAGGCTCAGCGGC |
| 425 | | TCCCAGGTGCGGGAGAGAGGCCTGCTGAAAATGACTGAATATAAACTTGTGGTA |
| | | GTTGGAGCTGGTGGCGTAGGCAAGAGTGCCTTGACGATACAGCTAATTCAGAAT |
| | | CATTTTGTGGACGAATATGATCCAACAATAGAGGATTCCTACAGGAAGCAAGTA |
| | | GTAATTGATGGAGAAACCTGTCTCTTGGATATTCTCGACACAGCAGGTCAAGAG |
| | | GAGTACAGTGCAATGAGGGACCAGTACATGAGGACTGGGGAGGGCTTTCTTTGT |
| | | GTATTTGCCATAAATAATACTAAATCATTTGAAGATATTCACCATTATAGAGAAC |
| | | AAATTAAAAGAGTTAAGGACTCTGAAGATGTACCTATGGTCCTAGTAGGAAATA |
| | | AATGTGATTTGCCTTCTAGAACAGTAGACACAAAACAGGCTCAGGACTTAGCAA |
| | | GAAGTTATGGAATTCCTTTTATTGAAACATCAGCAAAGACAAGACAGGGTGTTG |
| | | ATGATGCCTTCTATACATTAGTTCGAGAAATTCGAAAACATAAAGAAAAGATGA |
| | | GCAAAGATGGTAAAAAGAAGAAAAAGAAGTCAAAGACAAAGTGTGTAATTATG |
| | | TAAATACAATTTGTACTTTTTTCTTAAGGCATACTAGTACAAGTGGTAATTTTTGT |
| | | ACATTACACTAAATTATTAGCATTTGTTTTAGCATTACCTAATTTTTTTCCTGCTC |
| | | CATGCAGACTGTTAGCTTTTACCTTAAATGCTTATTTTAAAATGACAGTGGAAGT |
| | | TTTTTTTTCCTCTAAGTGCCAGTATTCCCAGAGTTTTGGTTTTTGAACTAGCAATG |
| | | CCTGTGAAAAAGAAACTGAATACCTAAGATTTCTGTCTTGGGGCTTTTGGTGCAT |
| | | GCAGTTGATTACTTCTTATTTTTCTTACCAATTGTGAATGTTGGTGTGAAACAAAT |
| | | TAATGAAGCTTTTGAATCATCCCTATTCTGTGTTTTATCTAGTCACATAAATGGAT |
| | | TAATTACTAATTTCAGTTGAGACCTTCTAATTGGTTTTTACTGAAACATTGAGGG |
| | | AACACAAATTTATGGGCTTCCTGATGATGATTCTTCTAGGCATCATGTCCTATAG |
| | | TTTGTCATCCCTGATGAATGTAAAGTTACACTGTTCACAAAGGTTTTGTCTCCTTT |
| | | CCACTGCTATTAGTCATGGTCACTCTCCCCAAAATATTATATTTTTTCTATAAAAA |
| | | GAAAAAAATGGAAAAAAATTACAAGGCAATGGAAACTATTATAAGGCCATTTCC |
| | | TTTTCACATTAGATAAATTACTATAAAGACTCCTAATAGCTTTTCCTGTTAAGGCA |
| | | GACCCAGTATGAAATGGGGATTATTATAGCAACCATTTTGGGGCTATATTTACAT |
| | | GCTACTAAATTTTTATAATAATTGAAAAGATTTTAACAAGTATAAAAAATTCTCA |
| | | TAGGAATTAAATGTAGTCTCCCTGTGTCAGACTGCTCTTTCATAGTATAACTTTAA |
| | | ATCTTTTCTTCAACTTGAGTCTTTGAAGATAGTTTTAATTCTGCTTGTGACATTAA |
| | | AAGATTATTTGGGCCAGTTATAGCTTATTAGGTGTTGAAGAGACCAAGGTTGCAA |
| | | GGCCAGGCCCTGTGTGAACCTTTGAGCTTTCATAGAGAGTTTCACAGCATGGACT |
| | | GTGTCCCCACGGTCATCCAGTGTTGTCATGCATTGGTTAGTCAAAATGGGGAGGG |
| | | ACTAGGGCAGTTTGGATAGCTCAACAAGATACAATCTCACTCTGTGGTGGTCCTG |
| | | CTGACAAATCAAGAGCATTGCTTTTGTTTCTTAAGAAAACAAACTCTTTTTTAAA |
| | | AATTACTTTTAAATATTAACTCAAAAGTTGAGATTTTGGGGTGGTGGTGTGCCAA |
| | | GACATTAATTTTTTTTTTAAACAATGAAGTGAAAAAGTTTTACAATCTCTAGGTTT |
| | | GGCTAGTTCTCTTAACACTGGTTAAATTAACATTGCATAAACACTTTTCAAGTCT |
| | | GATCCATATTTAATAATGCTTTAAAATAAAAATAAAAACAATCCTTTTGATAAAT |
| | | TTAAAATGTTACTTATTTTAAAATAAATGAAGTGAGATGGCATGGTGAGGTGAA |
| | | AGTATCACTGGACTAGGAAGAAGGTGACTTAGGTTCTAGATAGGTGTCTTTTAGG |
| | | ACTCTGATTTTGAGGACATCACTTACTATCCATTTCTTCATGTTAAAAGAAGTCAT |
| | | CTCAAACTCTTAGTTTTTTTTTTTTACAACTATGTAATTTATATTCCATTTACATAA |
| | | GGATACACTTATTTGTCAAGCTCAGCACAATCTGTAAATTTTTAACCTATGTTAC |
| | | ACCATCTTCAGTGCCAGTCTTGGGCAAAATTGTGCAAGAGGTGAAGTTTATATTT |
| | | GAATATCCATTCTCGTTTTAGGACTCTTCTTCCATATTAGTGTCATCTTGCCTCCC |
| | | TACCTTCCACATGCCCCATGACTTGATGCAGTTTTAATACTTGTAATTCCCCTAAC |
| | | CATAAGATTTACTGCTGCTGTGGATATCTCCATGAAGTTTTCCCACTGAGTCACA |
| | | TCAGAAATGCCCTACATCTTATTTCCTCAGGGCTCAAGAGAATCTGACAGATACC |
| | | ATAAAGGGATTTGACCTAATCACTAATTTTCAGGTGGTGGCTGATGCTTTGAACA |
| | | TCTCTTTGCTGCCCAATCCATTAGCGACAGTAGGATTTTTCAAACCTGGTATGAA |
| | | TAGACAGAACCCTATCCAGTGGAAGGAGAATTTAATAAAGATAGTGCTGAAAGA |
| | | ATTCCTTAGGTAATCTATAACTAGGACTACTCCTGGTAACAGTAATACATTCCAT |
| | | TGTTTTAGTAACCAGAAATCTTCATGCAATGAAAAATACTTTAATTCATGAAGCT |
| | | TACTTTTTTTTTTTGGTGTCAGAGTCTCGCTCTTGTCACCCAGGCTGGAATGCAGT |
| | | GGCGCCATCTCAGCTCACTGCAACCTCCATCTCCCAGGTTCAAGCGATTCTCGTG |
| | | CCTCGGCCTCCTGAGTAGCTGGGATTACAGGCGTGTGCCACTACACTCAACTAAT |
| | | TTTTGTATTTTTAGGAGAGACGGGGTTTCACCCTGTTGGCCAGGCTGGTCTCGAA |
| | | CTCCTGACCTCAAGTGATTCACCCACCTTGGCCTCATAAACCTGTTTTGCAGAAC |
| | | TCATTTATTCAGCAAATATTTATTGAGTGCCTACCAGATGCCAGTCACCACACAA |
| | | GGCACTGGGTATATGGTATCCCCAAACAAGAGACATAATCCCGGTCCTTAGGTA |
| | | GTGCTAGTGTGGTCTGTAATATCTTACTAAGGCCTTTGGTATACGACCCAGAGAT |
| | | AACACGATGCGTATTTTAGTTTTGCAAAGAAGGGGTTTGGTCTCTGTGCCAGCTC |
| | | TATAATTGTTTTGCTACGATTCCACTGAAACTCTTCGATCAAGCTACTTTATGTAA |
| | | ATCACTTCATTGTTTTAAAGGAATAAACTTGATTATATTGTTTTTTTATTTGGCAT |
| | | AACTGTGATTCTTTTAGGACAATTACTGTACACATTAAGGTGTATGTCAGATATT |
| | | CATATTGACCCAAATGTGTAATATTCCAGTTTTCTCTGCATAAGTAATTAAAATA |
| | | TACTTAAAAATTAATAGTTTTATCTGGGTACAAATAAACAGGTGCCTGAACTAGT |
| | | TCACAGACAAGGAAACTTCTATGTAAAAATCACTATGATTTCTGAATTGCTATGT |
| | | GAAACTACAGATCTTTGGAACACTGTTTAGGTAGGGTGTTAAGACTTACACAGTA |
| | | CCTCGTTTCTACACAGAGAAAGAAATGGCCATACTTCAGGAACTGCAGTGCTTAT |
| | | GAGGGGATATTTAGGCCTCTTGAATTTTTGATGTAGATGGGCATTTTTTTAAGGT |
| | | AGTGGTTAATTACCTTTATGTGAACTTTGAATGGTTTAACAAAAGATTTGTTTTTG |
| | | TAGAGATTTTAAAGGGGGAGAATTCTAGAAATAAATGTTACCTAATTATTACAGC |
| | | CTTAAAGACAAAAATCCTTGTTGAAGTTTTTTTAAAAAAAGCTAAATTACATAGA |
| | | CTTAGGCATTAACATGTTTGTGGAAGAATATAGCAGACGTATATTGTATCATTTG |
| | | AGTGAATGTTCCCAAGTAGGCATTCTAGGCTCTATTTAACTGAGTCACACTGCAT |
| | | AGGAATTTAGAACCTAACTTTTATAGGTTATCAAAACTGTTGTCACCATTGCACA |
| | | ATTTTGTCCTAATATATACATAGAAACTTTGTGGGGCATGTTAAGTTACAGTTTG |
| | | CACAAGTTCATCTCATTTGTATTCCATTGATTTTTTTTTTCTTCTAAACATTTTTTC |
| | | TTCAAACAGTATATAACTTTTTTTAGGGGATTTTTTTTTAGACAGCAAAAACTATC |
| | | TGAAGATTTCCATTTGTCAAAAAGTAATGATTTCTTGATAATTGTGTAGTAATGTT |
| | | TTTTAGAACCCAGCAGTTACCTTAAAGCTGAATTTATATTTAGTAACTTCTGTGTT |
| | | AATACTGGATAGCATGAATTCTGCATTGAGAAACTGAATAGCTGTCATAAAATG |
| | | AAACTTTCTTTCTAAAGAAAGATACTCACATGAGTTCTTGAAGAATAGTCATAAC |
| | | TAGATTAAGATCTGTGTTTTAGTTTAATAGTTTGAAGTGCCTGTTTGGGATAATG |
| | | ATAGGTAATTTAGATGAATTTAGGGGAAAAAAAAGTTATCTGCAGATATGTTGA |
| | | GGGCCCATCTCTCCCCCCACACCCCCACAGAGCTAACTGGGTTACAGTGTTTTAT |
| | | CCGAAAGTTTCCAATTCCACTGTCTTGTGTTTTCATGTTGAAAATACTTTTGCATT |
| | | TTTCCTTTGAGTGCCAATTTCTTACTAGTACTATTTCTTAATGTAACATGTTTACCT |
| | | GGAATGTATTTTAACTATTTTTGTATAGTGTAAACTGAAACATGCACATTTTGTAC |
| | | ATTGTGCTTTCTTTTGTGGGACATATGCAGTGTGATCCAGTTGTTTTCCATCATTT |
| | | GGTTGCGCTGACCTAGGAATGTTGGTCATATCAAACATTAAAAATGACCACTCTT |
| | | TTAATTGAAATTAACTTTTAAATGTTTATAGGAGTATGTGCTGTGAAGTGATCTA |
| | | AAATTTGTAATATTTTTGTCATGAACTGTACTACTCCTAATTATTGTAATGTAATA |
| | | AAAATAGTTACAGTGAC |
| SEQ | KRAS | CTAGGCGGCGGCCGCGGCGGCGGAGGCAGCAGCGGCGGCGGCAGTGGCGGCGG |
| ID | | CGAAGGTGGCGGCGGCTCGGCCAGTACTCCCGGCCCCCGCCATTTCGGACTGGG |
| NO: | | AGCGAGCGCGGCGCAGGCACTGAAGGCGGCGGCGGGGCCAGAGGCTCAGCGGC |
| 426 | | TCCCAGGTGCGGGAGAGAGGCCTGCTGAAAATGACTGAATATAAACTTGTGGTA |
| | | GTTGGAGCTGGTGGCGTAGGCAAGAGTGCCTTGACGATACAGCTAATTCAGAAT |
| | | CATTTTGTGGACGAATATGATCCAACAATAGAGGATTCCTACAGGAAGCAAGTA |
| | | GTAATTGATGGAGAAACCTGTCTCTTGGATATTCTCGACACAGCAGGTCAAGAG |
| | | GAGTACAGTGCAATGAGGGACCAGTACATGAGGACTGGGGAGGGCTTTCTTTGT |
| | | GTATTTGCCATAAATAATACTAAATCATTTGAAGATATTCACCATTATAGAGAAC |
| | | AAATTAAAAGAGTTAAGGACTCTGAAGATGTACCTATGGTCCTAGTAGGAAATA |
| | | AATGTGATTTGCCTTCTAGAACAGTAGACACAAAACAGGCTCAGGACTTAGCAA |
| | | GAAGTTATGGAATTCCTTTTATTGAAACATCAGCAAAGACAAGACAGAGAGTGG |
| | | AGGATGCTTTTTATACATTGGTGAGAGAGATCCGACAATACAGATTGAAAAAAA |
| | | TCAGCAAAGAAGAAAAGACTCCTGGCTGTGTGAAAATTAAAAAATGCATTATAA |
| | | TGTAATCTGGGTGTTGATGATGCCTTCTATACATTAGTTCGAGAAATTCGAAAAC |
| | | ATAAAGAAAAGATGAGCAAAGATGGTAAAAAGAAGAAAAAGAAGTCAAAGACA |
| | | AAGTGTGTAATTATGTAAATACAATTTGTACTTTTTTCTTAAGGCATACTAGTACA |
| | | AGTGGTAATTTTTGTACATTACACTAAATTATTAGCATTTGTTTTAGCATTACCTA |
| | | ATTTTTTTCCTGCTCCATGCAGACTGTTAGCTTTTACCTTAAATGCTTATTTTAAA |
| | | ATGACAGTGGAAGTTTTTTTTTCCTCTAAGTGCCAGTATTCCCAGAGTTTTGGTTT |
| | | TTGAACTAGCAATGCCTGTGAAAAAGAAACTGAATACCTAAGATTTCTGTCTTGG |
| | | GGCTTTTGGTGCATGCAGTTGATTACTTCTTATTTTTCTTACCAATTGTGAATGTT |
| | | GGTGTGAAACAAATTAATGAAGCTTTTGAATCATCCCTATTCTGTGTTTTATCTAG |
| | | TCACATAAATGGATTAATTACTAATTTCAGTTGAGACCTTCTAATTGGTTTTTACT |
| | | GAAACATTGAGGGAACACAAATTTATGGGCTTCCTGATGATGATTCTTCTAGGCA |
| | | TCATGTCCTATAGTTTGTCATCCCTGATGAATGTAAAGTTACACTGTTCACAAAG |
| | | GTTTTGTCTCCTTTCCACTGCTATTAGTCATGGTCACTCTCCCCAAAATATTATAT |
| | | TTTTTCTATAAAAAGAAAAAAATGGAAAAAAATTACAAGGCAATGGAAACTATT |
| | | ATAAGGCCATTTCCTTTTCACATTAGATAAATTACTATAAAGACTCCTAATAGCT |
| | | TTTCCTGTTAAGGCAGACCCAGTATGAAATGGGGATTATTATAGCAACCATTTTG |
| | | GGGCTATATTTACATGCTACTAAATTTTTATAATAATTGAAAAGATTTTAACAAG |
| | | TATAAAAAATTCTCATAGGAATTAAATGTAGTCTCCCTGTGTCAGACTGCTCTTT |
| | | CATAGTATAACTTTAAATCTTTTCTTCAACTTGAGTCTTTGAAGATAGTTTTAATT |
| | | CTGCTTGTGACATTAAAAGATTATTTGGGCCAGTTATAGCTTATTAGGTGTTGAA |
| | | GAGACCAAGGTTGCAAGGCCAGGCCCTGTGTGAACCTTTGAGCTTTCATAGAGA |
| | | GTTTCACAGCATGGACTGTGTCCCCACGGTCATCCAGTGTTGTCATGCATTGGTT |
| | | AGTCAAAATGGGGAGGGACTAGGGCAGTTTGGATAGCTCAACAAGATACAATCT |
| | | CACTCTGTGGTGGTCCTGCTGACAAATCAAGAGCATTGCTTTTGTTTCTTAAGAA |
| | | AACAAACTCTTTTTTAAAAATTACTTTTAAATATTAACTCAAAAGTTGAGATTTTG |
| | | GGGTGGTGGTGTGCCAAGACATTAATTTTTTTTTTAAACAATGAAGTGAAAAAGT |
| | | TTTACAATCTCTAGGTTTGGCTAGTTCTCTTAACACTGGTTAAATTAACATTGCAT |
| | | AAACACTTTTCAAGTCTGATCCATATTTAATAATGCTTTAAAATAAAAATAAAAA |
| | | CAATCCTTTTGATAAATTTAAAATGTTACTTATTTTAAAATAAATGAAGTGAGAT |
| | | GGCATGGTGAGGTGAAAGTATCACTGGACTAGGAAGAAGGTGACTTAGGTTCTA |
| | | GATAGGTGTCTTTTAGGACTCTGATTTTGAGGACATCACTTACTATCCATTTCTTC |
| | | ATGTTAAAAGAAGTCATCTCAAACTCTTAGTTTTTTTTTTTTACAACTATGTAATT |
| | | TATATTCCATTTACATAAGGATACACTTATTTGTCAAGCTCAGCACAATCTGTAA |
| | | ATTTTTAACCTATGTTACACCATCTTCAGTGCCAGTCTTGGGCAAAATTGTGCAA |
| | | GAGGTGAAGTTTATATTTGAATATCCATTCTCGTTTTAGGACTCTTCTTCCATATT |
| | | AGTGTCATCTTGCCTCCCTACCTTCCACATGCCCCATGACTTGATGCAGTTTTAAT |
| | | ACTTGTAATTCCCCTAACCATAAGATTTACTGCTGCTGTGGATATCTCCATGAAG |
| | | TTTTCCCACTGAGTCACATCAGAAATGCCCTACATCTTATTTCCTCAGGGCTCAA |
| | | GAGAATCTGACAGATACCATAAAGGGATTTGACCTAATCACTAATTTTCAGGTGG |
| | | TGGCTGATGCTTTGAACATCTCTTTGCTGCCCAATCCATTAGCGACAGTAGGATT |
| | | TTTCAAACCTGGTATGAATAGACAGAACCCTATCCAGTGGAAGGAGAATTTAAT |
| | | AAAGATAGTGCTGAAAGAATTCCTTAGGTAATCTATAACTAGGACTACTCCTGGT |
| | | AACAGTAATACATTCCATTGTTTTAGTAACCAGAAATCTTCATGCAATGAAAAAT |
| | | ACTTTAATTCATGAAGCTTACTTTTTTTTTTTGGTGTCAGAGTCTCGCTCTTGTCAC |
| | | CCAGGCTGGAATGCAGTGGCGCCATCTCAGCTCACTGCAACCTCCATCTCCCAGG |
| | | TTCAAGCGATTCTCGTGCCTCGGCCTCCTGAGTAGCTGGGATTACAGGCGTGTGC |
| | | CACTACACTCAACTAATTTTTGTATTTTTAGGAGAGACGGGGTTTCACCCTGTTG |
| | | GCCAGGCTGGTCTCGAACTCCTGACCTCAAGTGATTCACCCACCTTGGCCTCATA |
| | | AACCTGTTTTGCAGAACTCATTTATTCAGCAAATATTTATTGAGTGCCTACCAGA |
| | | TGCCAGTCACCACACAAGGCACTGGGTATATGGTATCCCCAAACAAGAGACATA |
| | | ATCCCGGTCCTTAGGTAGTGCTAGTGTGGTCTGTAATATCTTACTAAGGCCTTTG |
| | | GTATACGACCCAGAGATAACACGATGCGTATTTTAGTTTTGCAAAGAAGGGGTTT |
| | | GGTCTCTGTGCCAGCTCTATAATTGTTTTGCTACGATTCCACTGAAACTCTTCGAT |
| | | CAAGCTACTTTATGTAAATCACTTCATTGTTTTAAAGGAATAAACTTGATTATATT |
| | | GTTTTTTTATTTGGCATAACTGTGATTCTTTTAGGACAATTACTGTACACATTAAG |
| | | GTGTATGTCAGATATTCATATTGACCCAAATGTGTAATATTCCAGTTTTCTCTGCA |
| | | TAAGTAATTAAAATATACTTAAAAATTAATAGTTTTATCTGGGTACAAATAAACA |
| | | GGTGCCTGAACTAGTTCACAGACAAGGAAACTTCTATGTAAAAATCACTATGATT |
| | | TCTGAATTGCTATGTGAAACTACAGATCTTTGGAACACTGTTTAGGTAGGGTGTT |
| | | AAGACTTACACAGTACCTCGTTTCTACACAGAGAAAGAAATGGCCATACTTCAG |
| | | GAACTGCAGTGCTTATGAGGGGATATTTAGGCCTCTTGAATTTTTGATGTAGATG |
| | | GGCATTTTTTTAAGGTAGTGGTTAATTACCTTTATGTGAACTTTGAATGGTTTAAC |
| | | AAAAGATTTGTTTTTGTAGAGATTTTAAAGGGGGAGAATTCTAGAAATAAATGTT |
| | | ACCTAATTATTACAGCCTTAAAGACAAAAATCCTTGTTGAAGTTTTTTTAAAAAA |
| | | AGCTAAATTACATAGACTTAGGCATTAACATGTTTGTGGAAGAATATAGCAGAC |
| | | GTATATTGTATCATTTGAGTGAATGTTCCCAAGTAGGCATTCTAGGCTCTATTTAA |
| | | CTGAGTCACACTGCATAGGAATTTAGAACCTAACTTTTATAGGTTATCAAAACTG |
| | | TTGTCACCATTGCACAATTTTGTCCTAATATATACATAGAAACTTTGTGGGGCAT |
| | | GTTAAGTTACAGTTTGCACAAGTTCATCTCATTTGTATTCCATTGATTTTTTTTTTC |
| | | TTCTAAACATTTTTTCTTCAAACAGTATATAACTTTTTTTAGGGGATTTTTTTTTAG |
| | | ACAGCAAAAACTATCTGAAGATTTCCATTTGTCAAAAAGTAATGATTTCTTGATA |
| | | ATTGTGTAGTAATGTTTTTTAGAACCCAGCAGTTACCTTAAAGCTGAATTTATATT |
| | | TAGTAACTTCTGTGTTAATACTGGATAGCATGAATTCTGCATTGAGAAACTGAAT |
| | | AGCTGTCATAAAATGAAACTTTCTTTCTAAAGAAAGATACTCACATGAGTTCTTG |
| | | AAGAATAGTCATAACTAGATTAAGATCTGTGTTTTAGTTTAATAGTTTGAAGTGC |
| | | CTGTTTGGGATAATGATAGGTAATTTAGATGAATTTAGGGGAAAAAAAAGTTAT |
| | | CTGCAGATATGTTGAGGGCCCATCTCTCCCCCCACACCCCCACAGAGCTAACTGG |
| | | GTTACAGTGTTTTATCCGAAAGTTTCCAATTCCACTGTCTTGTGTTTTCATGTTGA |
| | | AAATACTTTTGCATTTTTCCTTTGAGTGCCAATTTCTTACTAGTACTATTTCTTAAT |
| | | GTAACATGTTTACCTGGAATGTATTTTAACTATTTTTGTATAGTGTAAACTGAAAC |
| | | ATGCACATTTTGTACATTGTGCTTTCTTTTGTGGGACATATGCAGTGTGATCCAGT |
| | | TGTTTTCCATCATTTGGTTGCGCTGACCTAGGAATGTTGGTCATATCAAACATTAA |
| | | AAATGACCACTCTTTTAATTGAAATTAACTTTTAAATGTTTATAGGAGTATGTGCT |
| | | GTGAAGTGATCTAAAATTTGTAATATTTTTGTCATGAACTGTACTACTCCTAATTA |
| | | TTGTAATGTAATAAAAATAGTTACAGTGAC |
| SEQ | ICAM1 | GAGCTCCTCTGCTACTCAGAGTTGCAACCTCAGCCTCGCTATGGCTCCCAGCAGC |
| ID | | CCCCGGCCCGCGCTGCCCGCACTCCTGGTCCTGCTCGGGGCTCTGTTCCCAGGAC |
| NO: | | CTGGCAATGCCCAGACATCTGTGTCCCCCTCAAAAGTCATCCTGCCCCGGGGAGG |
| 427 | | CTCCGTGCTGGTGACATGCAGCACCTCCTGTGACCAGCCCAAGTTGTTGGGCATA |
| | | GAGACCCCGTTGCCTAAAAAGGAGTTGCTCCTGCCTGGGAACAACCGGAAGGTG |
| | | TATGAACTGAGCAATGTGCAAGAAGATAGCCAACCAATGTGCTATTCAAACTGC |
| | | CCTGATGGGCAGTCAACAGCTAAAACCTTCCTCACCGTGTACTGGACTCCAGAAC |
| | | GGGTGGAACTGGCACCCCTCCCCTCTTGGCAGCCAGTGGGCAAGAACCTTACCCT |
| | | ACGCTGCCAGGTGGAGGGTGGGGCACCCCGGGCCAACCTCACCGTGGTGCTGCT |
| | | CCGTGGGGAGAAGGAGCTGAAACGGGAGCCAGCTGTGGGGGAGCCCGCTGAGG |
| | | TCACGACCACGGTGCTGGTGAGGAGAGATCACCATGGAGCCAATTTCTCGTGCC |
| | | GCACTGAACTGGACCTGCGGCCCCAAGGGCTGGAGCTGTTTGAGAACACCTCGG |
| | | CCCCCTACCAGCTCCAGACCTTTGTCCTGCCAGCGACTCCCCCACAACTTGTCAG |
| | | CCCCCGGGTCCTAGAGGTGGACACGCAGGGGACCGTGGTCTGTTCCCTGGACGG |
| | | GCTGTTCCCAGTCTCGGAGGCCCAGGTCCACCTGGCACTGGGGGACCAGAGGTT |
| | | GAACCCCACAGTCACCTATGGCAACGACTCCTTCTCGGCCAAGGCCTCAGTCAGT |
| | | GTGACCGCAGAGGACGAGGGCACCCAGCGGCTGACGTGTGCAGTAATACTGGGG |
| | | AACCAGAGCCAGGAGACACTGCAGACAGTGACCATCTACAGCTTTCCGGCGCCC |
| | | AACGTGATTCTGACGAAGCCAGAGGTCTCAGAAGGGACCGAGGTGACAGTGAAG |
| | | TGTGAGGCCCACCCTAGAGCCAAGGTGACGCTGAATGGGGTTCCAGCCCAGCCA |
| | | CTGGGCCCGAGGGCCCAGCTCCTGCTGAAGGCCACCCCAGAGGACAACGGGCGC |
| | | AGCTTCTCCTGCTCTGCAACCCTGGAGGTGGCCGGCCAGCTTATACACAAGAACC |
| | | AGACCCGGGAGCTTCGTGTCCTGTATGGCCCCCGACTGGACGAGAGGGATTGTC |
| | | CGGGAAACTGGACGTGGCCAGAAAATTCCCAGCAGACTCCAATGTGCCAGGCTT |
| | | GGGGGAACCCATTGCCCGAGCTCAAGTGTCTAAAGGATGGCACTTTCCCACTGCC |
| | | CATCGGGGAATCAGTGACTGTCACTCGAGATCTTGAGGGCACCTACCTCTGTCGG |
| | | GCCAGGAGCACTCAAGGGGAGGTCACCCGCAAGGTGACCGTGAATGTGCTCTCC |
| | | CCCCGGTATGAGATTGTCATCATCACTGTGGTAGCAGCCGCAGTCATAATGGGCA |
| | | CTGCAGGCCTCAGCACGTACCTCTATAACCGCCAGCGGAAGATCAAGAAATACA |
| | | GACTACAACAGGCCCAAAAAGGGACCCCCATGAAACCGAACACACAAGCCACG |
| | | CCTCCCTGAACCTATCCCGGGACAGGGCCTCTTCCTCGGCCTTCCCATATTGGTG |
| | | GCAGTGGTGCCACACTGAACAGAGTGGAAGACATATGCCATGCAGCTACACCTA |
| | | CCGGCCCTGGGACGCCGGAGGACAGGGCATTGTCCTCAGTCAGATACAACAGCA |
| | | TTTGGGGCCATGGTACCTGCACACCTAAAACACTAGGCCACGCATCTGATCTGTA |
| | | GTCACATGACTAAGCCAAGAGGAAGGAGCAAGACTCAAGACATGATTGATGGAT |
| | | GTTAAAGTCTAGCCTGATGAGAGGGGAAGTGGTGGGGGAGACATAGCCCCACCA |
| | | TGAGGACATACAACTGGGAAATACTGAAACTTGCTGCCTATTGGGTATGCTGAG |
| | | GCCCCACAGACTTACAGAAGAAGTGGCCCTCCATAGACATGTGTAGCATCAAAA |
| | | CACAAAGGCCCACACTTCCTGACGGATGCCAGCTTGGGCACTGCTGTCTACTGAC |
| | | CCCAACCCTTGATGATATGTATTTATTCATTTGTTATTTTACCAGCTATTTATTGA |
| | | GTGTCTTTTATGTAGGCTAAATGAACATAGGTCTCTGGCCTCACGGAGCTCCCAG |
| | | TCCTAATCACATTCAAGGTCACCAGGTACAGTTGTACAGGTTGTACACTGCAGGA |
| | | GAGTGCCTGGCAAAAAGATCAAATGGGGCTGGGACTTCTCATTGGCCAACCTGC |
| | | CTTTCCCCAGAAGGAGTGATTTTTCTATCGGCACAAAAGCACTATATGGACTGGT |
| | | AATGGTTACAGGTTCAGAGATTACCCAGTGAGGCCTTATTCCTCCCTTCCCCCCA |
| | | AAACTGACACCTTTGTTAGCCACCTCCCCACCCACATACATTTCTGCCAGTGTTC |
| | | ACAATGACACTCAGCGGTCATGTCTGGACATGAGTGCCCAGGGAATATGCCCAA |
| | | GCTATGCCTTGTCCTCTTGTCCTGTTTGCATTTCACTGGGAGCTTGCACTATGCAG |
| | | CTCCAGTTTCCTGCAGTGATCAGGGTCCTGCAAGCAGTGGGGAAGGGGGCCAAG |
| | | GTATTGGAGGACTCCCTCCCAGCTTTGGAAGCCTCATCCGCGTGTGTGTGTGTGT |
| | | GTATGTGTAGACAAGCTCTCGCTCTGTCACCCAGGCTGGAGTGCAGTGGTGCAAT |
| | | CATGGTTCACTGCAGTCTTGACCTTTTGGGCTCAAGTGATCCTCCCACCTCAGCCT |
| | | CCTGAGTAGCTGGGACCATAGGCTCACAACACCACACCTGGCAAATTTGATTTTT |
| | | TTTTTTTTTCCAGAGACGGGGTCTCGCAACATTGCCCAGACTTCCTTTGTGTTAGT |
| | | TAATAAAGCTTTCTCAACTGCC |
| SEQ | IFNG | ACATTGTTCTGATCATCTGAAGATCAGCTATTAGAAGAGAAAGATCAGTTAAGTC |
| ID | | CTTTGGACCTGATCAGCTTGATACAAGAACTACTGATTTCAACTTCTTTGGCTTAA |
| NO: | | TTCTCTCGGAAACGATGAAATATACAAGTTATATCTTGGCTTTTCAGCTCTGCATC |
| 428 | | GTTTTGGGTTCTCTTGGCTGTTACTGCCAGGACCCATATGTAAAAGAAGCAGAAA |
| | | ACCTTAAGAAATATTTTAATGCAGGTCATTCAGATGTAGCGGATAATGGAACTCT |
| | | TTTCTTAGGCATTTTGAAGAATTGGAAAGAGGAGAGTGACAGAAAAATAATGCA |
| | | GAGCCAAATTGTCTCCTTTTACTTCAAACTTTTTAAAAACTTTAAAGATGACCAG |
| | | AGCATCCAAAAGAGTGTGGAGACCATCAAGGAAGACATGAATGTCAAGTTTTTC |
| | | AATAGCAACAAAAAGAAACGAGATGACTTCGAAAAGCTGACTAATTATTCGGTA |
| | | ACTGACTTGAATGTCCAACGCAAAGCAATACATGAACTCATCCAAGTGATGGCT |
| | | GAACTGTCGCCAGCAGCTAAAACAGGGAAGCGAAAAAGGAGTCAGATGCTGTTT |
| | | CGAGGTCGAAGAGCATCCCAGTAATGGTTGTCCTGCCTGCAATATTTGAATTTTA |
| | | AATCTAAATCTATTTATTAATATTTAACATTATTTATATGGGGAATATATTTTTAG |
| | | ACTCATCAATCAAATAAGTATTTATAATAGCAACTTTTGTGTAATGAAAATGAAT |
| | | ATCTATTAATATATGTATTATTTATAATTCCTATATCCTGTGACTGTCTCACTTAA |
| | | TCCTTTGTTTTCTGACTAATTAGGCAAGGCTATGTGATTACAAGGCTTTATCTCAG |
| | | GGGCCAACTAGGCAGCCAACCTAAGCAAGATCCCATGGGTTGTGTGTTTATTTCA |
| | | CTTGATGATACAATGAACACTTATAAGTGAAGTGATACTATCCAGTTACTGCCGG |
| | | TTTGAAAATATGCCTGCAATCTGAGCCAGTGCTTTAATGGCATGTCAGACAGAAC |
| | | TTGAATGTGTCAGGTGACCCTGATGAAAACATAGCATCTCAGGAGATTTCATGCC |
| | | TGGTGCTTCCAAATATTGTTGACAACTGTGACTGTACCCAAATGGAAAGTAACTC |
| | | ATTTGTTAAAATTATCAATATCTAATATATATGAATAAAGTGTAAGTTCACAACT |
| | | A |

### Oligonucleotide and Target-Binding Agent Technologies

A number of technologies can be used to generate therapeutically active nucleotide sequences for use in peptide oligonucleotide complexes disclosed herein. Several have examples of molecules in the clinic or advanced clinical development and can be employed for the nucleotide portion within the peptide oligonucleotide complexes described herein. A nucleotide of a peptide oligonucleotide complex may bind to a target molecule (e.g., a target DNA, RNA, or protein) and modulate an activity of the target molecule. In this way, the nucleotide may function as a target-binding agent, also referred to as a targeted agent. Examples of nucleotides that may function as target-binding agents include nucleotide antisense RNAs, complementary RNAs, inhibitory RNAs, interfering RNAs, nuclear RNAs, antisense oligonucleotides, microRNAs, oligonucleotides complementary to natural antisense transcripts, small interfering RNAs, small nuclear RNAs, aptamers, gapmers, anti-miRs, splice blocker antisense oligonucleotides, and U1 adapters.

Nucleotides (e.g., oligonucleotides targeted to a specific sequence for its regulation) may enter into cells through complexation with a TfR-binding peptide to form a TfR-binding peptide oligonucleotide complex. The TfR-binding peptide oligonucleotide complex may then be endocytosed by TfR or may enter the cell by other mechanisms. The oligonucleotide, with or without complex to the TfR-binding peptide (e.g., after linker cleavage), may exit the endosome or lysosome slowly over time through no active mechanism or through mechanisms of endosomal escape or through other mechanisms. The peptide oligonucleotide complex may exit the endosome or lysosome. A fragment or cleavage product of the peptide oligonucleotide complex may exit the endosome or lysosome. The oligonucleotide, the peptide oligonucleotide complex, or any fragment thereof may enter the cytosol and may enter the nucleus.

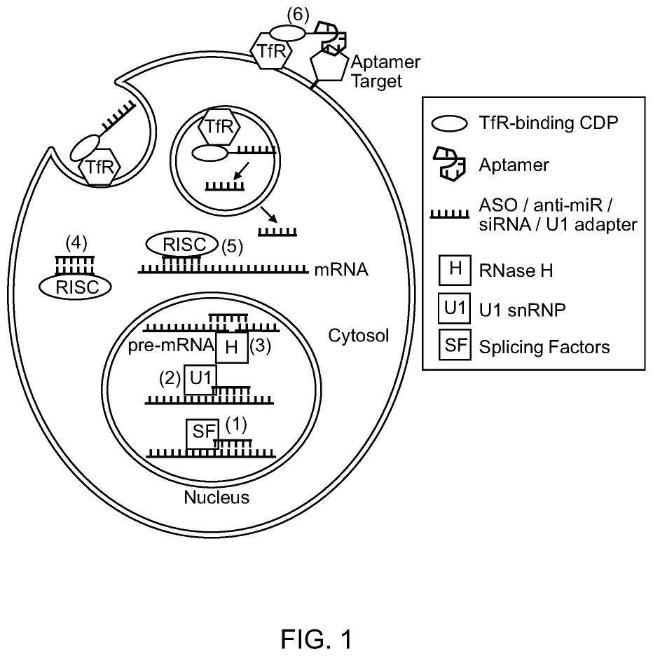

Possible mechanisms of action of oligonucleotides are illustrated in FIG. 1. In one embodiment, upon entry into the nucleus, oligonucleotides can (1) bind directly to mRNA structures and prevent the maturation (e.g., capping or splicing) of the targeted sequence, (2) modulate alternative splicing of a targeted sequence, (3) and recruit RNaseH1 to induce cleavage of a targeted sequence. In another embodiment, oligonucleotides in the cytoplasm can bind directly to the target mRNA and sterically block the ribosomal subunits from attaching and/or running along the mRNA transcript during translation hence resulting in lack of translation of the target sequence. In another embodiment, oligonucleotides can also be designed to (4) directly bind to microRNA (miRNA) sequences or natural antisense transcripts (NATs) sequences, each of the foregoing thereby prohibiting miRNAs and NATs from inhibiting their own specific RNA targets, which ultimately leads to reduced degradation or increased translation of one or more sequences themselves targeted by the miRNA or NAT. In another embodiment, siRNA (which may be targeted to a specific sequence and regulate expression of the target sequence) may alternatively be used to bind and regulate a targeted sequence in the cytoplasm (5), engaging an RNA-induced silencing complex (RISC), which is a multiprotein complex that incorporates one strand of a small interfering RNA (siRNA) or micro RNA (miRNA), using the siRNA or miRNA as a template for recognizing complementary mRNA of the targeted sequence. When it finds a complementary strand, its RNase domain cleaves the targeted sequence. In another embodiment, an aptamer (e.g., a nucleotide that modulates a specific protein or other target) may alternatively be used to bind and regulate a target (6). An aptamer (e.g., extracellular or intracellular) may function by directly binding and modulating activity of a protein target, for example by forming aptamer-protein interactions rather than through base pairing or hybridization interactions.

For example, conventional ASO, or antisense oligonucleotides, are typically 18-30 nucleotides (nt) in length. Several ASO therapeutic strategies exist, two of which (differing in their mechanism of target RNA interference) are further described. The first ASOs are sometimes called “Gapmers” because they have a central region with DNA-based-sugar nucleotides that are often (but not always) flanked by non-DNA-sugar nucleotides with greater resistance to nucleases. The DNA region, at least 4 nt in length but typically >6, causes a DNA/RNA hybrid that engages RNase H endonuclease to cleave the target RNA. Among clinically approved gapmers are fomivirsen and mipomersen. In some embodiments, a DNA region of a gapmer may comprise from about 4 to about 30, from about 4 to about 25, from about 4 to about 20, from about 4 to about 15, from about 4 to about 10, from about 6 to about 30, from about 6 to about 25, from about 6 to about 20, from about 6 to about 15, or from about 6 to about 10 nucleotide residues. In some embodiments, a non-DNA region of a gapmer may comprise about 1, 2, 3, 4, 5, 6, 7, 8, 9, or 10 nucleotide residues 3′ of the DNA region. In some embodiments, a non-DNA region of a gapmer may comprise about 1, 2, 3, 4, 5, 6, 7, 8, 9, or 10 nucleotide residues 5′ of the DNA region.

The second conventional ASO simply serves to bind to the target transcript, but not induce RNase degradation, so no DNA-based-sugars are used. Instead, binding is designed to disrupt processing into mature mRNA. One such activity relies on binding to the mRNA at or near splice sites to drive particular splice isoforms in the target RNA, resulting in modulating target RNA by disrupting mRNA splicing and resulting in exon skipping. These are commonly called “splice blocking” or “splice blocker” ASOs amongst other known names. One example is eteplirsen, designed to alter splicing of DMD (dystrophin) gene in Duchenne Muscular Dystrophy patients, correcting a mutation that would otherwise create a truncated and non-functional dystrophin by splicing out the mutant exons and creating a different truncated (but functional) protein to appear.

Another example is siRNA molecules which specifically interact with the canonical RNAi pathway (the RISC complex) to drive cleavage or steric blocking of hybridized transcripts; cleavage-vs-blocking depends on whether the match is perfect (cleavage) or imperfect but still stable (blocking). Length is typically a double-stranded RNA where the overlapping region is 19-22 and each strand has two extra nt at their 3′ ends. Chemistry is largely RNA-based-sugars, with some DNA-based sugars at the 3′ overhangs. Clinical examples include patisiran (targets TTR) and givosiran (targets ALAS1). In some embodiments, an overlapping region of a siRNA may comprise from about 10 to about 40, from about 10 to about 35, from about 10 to about 30, from about 10 to about 25, from about 10 to about 22, from about 10 to about 21, from about 10 to about 20, from about 15 to about 40, from about 15 to about 35, from about 15 to about 30, from about 15 to about 25, from about 15 to about 22, from about 15 to about 21, from about 15 to about 20, from about 17 to about 40, from about 17 to about 35, from about 17 to about 30, from about 17 to about 25, from about 17 to about 22, from about 17 to about 21, from about 17 to about 20, from about 18 to about 40, from about 18 to about 35, from about 18 to about 30, from about 18 to about 25, from about 18 to about 22, from about 18 to about 21, from about 18 to about 20, from about 19 to about 40, from about 19 to about 35, from about 19 to about 30, from about 19 to about 25, from about 19 to about 22, from about 19 to about 21, or from about 19 to about 20 nucleotide residues. In some embodiments, an overhang region may comprise about 1, 2, 3, 4, 5, 6, 7, 8, 9, or 10 nucleotide residues.

Another example are anti-miRs. Anti-miRs may function as steric blockers designed against miRNAs that would block a RISC complex loaded with a specific disease-associated miRNA without being subject to cleavage by the RISC complex RNase subunit. One clinical example is miravirsen, a 15-base oligo with a mixture of DNA and LNA sugars that targets miR-122 in hepatitis C patients. An anti-miR nucleotide may be of sufficient length to anneal specifically and stably to the target miR, but the length of the sequence may vary. For example, an anti-miR may have a length of up to about 21 nt, corresponding to the maximum size loaded into RISC. In some embodiments, an anti-miR nucleotide may comprise from about 10 to about 25, from about 10 to about 23, from about 10 to about 21, from about 10 to about 20, from about 10 to about 19, from about 10 to about 18, from about 13 to about 25, from about 13 to about 23, from about 13 to about 21, from about 13 to about 20, from about 13 to about 19, from about 13 to about 18, from about 15 to about 25, from about 15 to about 23, from about 15 to about 21, from about 15 to about 20, from about 15 to about 19, from about 15 to about 18, from about 16 to about 25, from about 16 to about 23, from about 16 to about 21, from about 16 to about 20, from about 16 to about 19, or from about 16 to about 18 nucleotide residues.

Another example is U1 adapters which have two parts. One anneals to the U1-snRNA of the U1-snRNP complex, and the other binds to the target RNA, bringing the U1-snRNP to the polyA site and inhibiting polyadenylation; absence of a polyA tail causes the mRNA to be degraded. The U1-binding region is at least 10 nt but up to 19 nt. Target binding region can be from about 15 nt to about 25 nt. Chemistry in early studies made heavy use of LNA and 2′-O-Methyl sugars. In some embodiments, a U1 binding region may comprise from about 10 to about 25, from about 10 to about 23, from about 10 to about 21, from about 10 to about 20, from about 10 to about 19, from about 10 to about 18, from about 13 to about 25, from about 13 to about 23, from about 13 to about 21, from about 13 to about 20, from about 13 to about 19, from about 13 to about 18, from about 15 to about 25, from about 15 to about 23, from about 15 to about 21, from about 15 to about 20, from about 15 to about 19, from about 15 to about 18, from about 16 to about 25, from about 16 to about 23, from about 16 to about 21, from about 16 to about 20, from about 16 to about 19, or from about 16 to about 18 nucleotide residues. In some embodiments, a target binding region may comprise from about 10 to about 40, from about 10 to about 35, from about 10 to about 30, from about 10 to about 25, from about 10 to about 22, from about 10 to about 21, from about 10 to about 20, from about 15 to about 40, from about 15 to about 35, from about 15 to about 30, from about 15 to about 25, from about 15 to about 22, from about 15 to about 21, from about 15 to about 20, from about 17 to about 40, from about 17 to about 35, from about 17 to about 30, from about 17 to about 25, from about 17 to about 22, from about 17 to about 21, from about 17 to about 20, from about 18 to about 40, from about 18 to about 35, from about 18 to about 30, from about 18 to about 25, from about 18 to about 22, from about 18 to about 21, from about 18 to about 20, from about 19 to about 40, from about 19 to about 35, from about 19 to about 30, from about 19 to about 25, from about 19 to about 22, from about 19 to about 21, or from about 19 to about 20 nucleotide residues.

Another example of a nucleotide of the present disclosure is an aptamer. Aptamers disrupt target activity using a mechanism that differs from other nucleotides described herein that form base pairing interactions with a target nucleotide. Aptamers are nucleic acids that form secondary structures (e.g., where a single strand of nucleic acid base-pairs with itself upon folding, creating loops in various locations). Aptamers may be screened for interaction with target proteins. Aptamers may have varied nucleotide chemistry and may include a mixture of conventional RNA and/or DNA sugars and modified sugars (e.g., 2′-O-Methyl (2′-O-Me) RNA or 2′-Fluoro (2′-F) RNA sugars). For example, one clinically approved aptamer, pegaptanib (a VEGF-binding aptamer), has a mixture of 2′-O-Methyl (2′-O-Me) RNA and 2′-Fluoro (2′-F) RNA sugars and regular RNA and DNA sugars. An aptamer sequence may be long enough to form a stable secondary structure (e.g., through intramolecular base pairing), but the length may vary. In some embodiments, an aptamer sequence may comprise from about 20 nt to about 40 nt. For example, experiments that identified pegaptanib used oligos of 20-40 nt in length. Shorter nucleotides (e.g., sequences shorter than about 40 nt) may be advantageous, as longer oligonucleotides may complicate nucleotide synthesis or engage the interferon response pathway. In some embodiments, an aptamer may comprise from about 15 to about 60, from about 15 to about 50, from about 15 to about 40, from about 15 to about 35, from about 15 to about 30, from about 20 to about 60, from about 20 to about 50, from about 20 to about 40, from about 20 to about 35, from about 20 to about 30, from about 25 to about 60, from about 25 to about 50, from about 25 to about 40, from about 25 to about 35, or from about 25 to about 30 nucleotide residues.

### Nucleotide and Oligo Delivery

A number of delivery methodologies can be used to link, fuse, complex or conjugate the nucleotide portion to the peptide portion of the peptide oligonucleotide complexes to generate therapeutically active peptide oligonucleotide complexes described herein. The nucleotide sequence in the complex may be single stranded (ssDNA, ssRNA), double stranded (dsDNA, dsRNA), a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter.

For example, the peptide (e.g., CDP, or TfR-binding peptide) may be linked, conjugated, complexed with, or fused to the nucleotide via various chemistries resulting in peptide oligonucleotide complexes that may form either a cleavable or stable linkage. For example, in some embodiments, a TfR-binding peptide may bind to TfR on the surface of cells, which may then be taken up via endocytosis into the early endosome. The nucleotide and peptide in the TfR-binding peptide oligonucleotide complex may either remain together (stable) or be cleaved apart (cleavable). If the linkage is stable, the TfR-binding peptide oligonucleotide complex may recycle back to the cell surface. For example, CNS access via transcytosis across the BBB may be performed by binding the peptide oligonucleotide complex to TfR followed by recycling the complex to the cell surface. Some of the TfR-binding peptide oligonucleotide complex may access low pH early endosomes. Once the nucleotides within the peptide oligonucleotide complex are exposed to endosome, they may remain and not be degraded due to stabilization chemistry such as by the oligonucleotide (backbone, sugar, linkage, etc.) variations described herein. If the TfR-binding peptide includes additional cell penetration capabilities, the peptide may facilitate accelerated escape of the oligonucleotide from the endosomal compartment into the cytosol. Even without added cell penetration capabilities, the oligonucleotides may slowly leak out of endosomes and access the cytosol.

Cleavage away from the TfR binder peptide within the peptide oligonucleotide complex may be advantageous in order to avoid repeated cycling to the cell surface or to facilitate endosomal escape of the oligonucleotide, in which case cleavable linkers may be used between the oligonucleotide and the peptide. The nucleotides within or cleaved from the peptide oligonucleotide complex may traffic, either actively or by passive diffusion, between the cytosol and nucleus. Some of the nucleotides within the peptide oligonucleotide complex can function within the nucleus of a cell, including gapmers, ASO splice blockers, and U1 adapters. Others function within the cytosol, including siRNA and anti-miRs. Aptamers are unique in that they do not function through hybridization or base paring interactions with nucleic acid targets. Instead, aptamers form secondary structures to bind to proteins or other macromolecules. Aptamers may function wherever the target protein or macromolecule is located. For example, if the target is on the surface of cells, cell penetration via endosomal accumulation may not be necessary, and it may be advantageous for linkers to be cleavable or non-cleavable depending on TfR-binding peptide trafficking and stability.

### Nucleotide Sequences

Nucleotides may be designed for use in the peptide nucleotide complexes of the present disclosure. In some embodiments, nucleotides that modify processing, translation, or other RNA functions (e.g., a gapmer, splice blocker, siRNA, anti-miR, or U1 adapter), have one or more of the following properties: (a) 8-50 nt in length, but preferably 12-30 nt in length. It is understood that any length of a nucleotide (nt) can be used within the foregoing ranges; (b) cross-species homology (e.g., by targeting highly-conserved motifs) is often a desirable feature but is not necessary for activity or clinical development; (c) avoidance of common SNPs in humans unless that SNP is involved in disease pathology (e.g., an allele-specific oligo) is often a desirable feature but is not necessary for activity or clinical development; (d) gene specificity (they have minimal homology to other sequences; for example, a sequence may have 3 or more mismatches to every other sequence). (e) avoid predicted secondary structures in both the oligo and the target region (there are software tools available to screen in silico for such secondary structure formation); (f) higher G/C content may be preferable, as G/C-rich sequences (e.g. CCAC, TCCC, GCCA) may be helpful for increasing affinity of the nucleotide to its target, whereas A/T-rich sequences (e.g. TAA) or runs of 4+ G (GGGG) may exhibit low or result in structural (G-quadruplex) formation. An oligonucleotide sequence can be 60%, 70%, 75%, 80%, 85%, 90%, 91%, 92%, 93%, 94%, 95%, 96%, 97%, 98%, 99%, or 100% complementarity or match to the target sequence. In some situations, an oligonucleotide with 100% complementarity will result in the target RNA being degraded. In some situations, an oligonucleotide that is less than 100% complementarity may not lead to degradation of the target RNA but may prevent translation and production of the encoded protein.

In some embodiments, gapmers have one or more of the following properties: (a) 12-30 nt in length. It is understood that any length of a nucleotide (nt) can be used within the foregoing range. (b) target sites are anywhere in the pre-mRNA, including UTRs, exons, or introns (c) central DNA region: minimum of 4 contiguous DNA nucleotides, often 10 or more are used. No artificial substitutions at 2′ site (e.g., 2′-O-methyl [2′-O-ME] or 2′-O-methoxyethyl [2′-O-MOE]) are tolerated due to requirements of RNase H recognition. (d) flanking region: can be DNA- or RNA-based-sugars. 2′ substitutions such as 2′-O-ME or 2′-O-MOE are tolerated. Linked nucleic acids (LNA) and morpholino (phosphorodiamidate morpholino oligo) chemistry are also acceptable in flanking region. (e) Backbone can be natural phosphodiester (PO) or non-natural phosphorothioate (PS) linkages. A clinical example is fomivirsen, a 21 nt gapmer wherein the whole oligo is PS-backbone DNA. Another example is mipomersen, a 20 nt gapmer wherein the entire backbone is PS linkages, and the central region uses DNA sugar flanked by 2′-O-MOE modified RNA. For these two examples, all C bases are 5-methyl-C, though this is not a strict requirement for engagement of RNase H1. Similarly, thiophosphorodiamidate chemistries may be used.

In some embodiments, steric blockers have one or more of the following properties; (a) as the molecule does not need to engage RNase H or any other enzyme, backbone and sugar chemistry can be more varied, (g) target sites for the nucleotide are complementary to one or more splice sites in the target RNA. A clinical example is eteplirsen, a 30 nt splice blocking ASO wherein whole oligonucleotide uses morpholino (Phosphorodiamidate morpholino oligo) chemistry. Another clinical example is nusinersen, an 18 nt ASO, whose backbone is entirely PS linked and uses 2′-O-MOE RNA chemistry. All C bases are 5-methyl-C, though this is not a strict requirement for engagement of RNase H1. Similarly, thiophosphorodiamidate chemistries may be used.

In some embodiments, siRNA have one or more of the following properties: (a) can be between 15 and 25 nt in length (between 13 to 23 nt overlap respectively), or up to 25 nt (23 nt overlap) per strand, but 21 nt (19 nt overlap) is common. It is understood that any length of a nucleotide (nt) overlap can be used within the foregoing ranges; (b) complements a sequence typically but not exclusively of 21-nt length in the target mRNA that typically but not exclusively begins with “AA” (c) target sites are ideally found in the mature spliced mRNA as the RISC complex for RNA cleavage is primarily cytosolic; (d) preferably but not exclusively avoids sequences within 100 nt of the mRNA start site, as the transcript at start site is more likely to be occupied by RNA polymerase, (e) successful siRNA constructs typically have more G/C at 5′ end of sense strand, more A/T at 3′ end of sense strand, and are roughly 30-60% in G/C content.

In some embodiments, anti-miR (anti-miRNA) have one or more of the following properties: (a) a perfect match to target sequence (specifically the 5′ end of the guide strand of the miRNA); (b) length can vary and can even be greater than the length of the mature guide strand. Screening for effective anti-miR constructs may begin with the shortest sequence that achieves specificity (no off-target homology) and increase length from there to empirically determine ideal minimal length for strong miRNA inhibition; (c) 2′ sugar modifications (2′-O-Me, 2′-O-MOE, 2′-F) and LNA sugars are commonly used. Sugars can be a mixture. A clinical example of an anti-miR is miravirsen, which uses a mixture of DNA and LNA sugars (d) PS linkages in backbone are common. PS linkages may reduce affinity, but sugar modifications may increase affinity.

In some embodiments, aptamers have one or more of the following properties: (a) length of aptamers can vary widely, as there is no biological complex (e.g., RISC) they interact with to function. Although composed of nucleic acids, they are more protein-like in function (e.g., bind to a target protein, etc.). The minimum length may be determined empirically to maintain sufficient stability of intra-strand hybridization to fold into a secondary structure, the upper limit on size is limited only by pharmacology, as longer sequences have a higher risk of engaging inflammatory pathways. Aptamer screening typically begins with libraries of 20-40 nt in length (not including flanking regions required for library amplification during screening); (b) as they form interactions via secondary structure rather than base pairing interactions, there are few limitations for their base patterns, since secondary structures are not only desirable but essential to their function. Design may be empirical for each target; (c) selection is typically via Systematic Evolution of Ligands by EXponential Enrichment (SELEX): random or semi-random sequences between primer-binding flanking regions are exposed to a target of interest on a solid substrate. The pooled oligonucleotide mixture is rinsed from the substrate, leaving only sequences that interact with the target remaining, and then binding sequences are eluted and amplified by PCR. (d) Sugar modifications commonly used include 2′-fluoro (2′-F), 2′-O-MOE, and 2′-O-Me, though other chemistries including (but not limited to) LNA and unlocked-nucleic-acids (UNA) are also possible; (e) backbones are typically PO or PS, but other linkages such as methylphosphonate are possible. A clinical aptamer example, pegaptanib, is entirely PO backbone, but others in development use other linkages. (f) aptamer termini are typically capped with unnatural nucleotide chemistries (e.g. 3′ inverted thymidine) or biotinylated nucleotides to reduce susceptibility to nucleases; (g) because activity is not based on base-pairing, aptamers can be much more creative with chemical modifications of the bases themselves; these can include bases designed to induce covalent bonds with target proteins to permanently disable them; (h) such modifications are tested after selection of an active, high affinity aptamer, as unmodified bases are required for nucleic acid amplification during SELEX (i) if the target protein is extracellular, less considerations are necessary than for cell penetration capabilities.

In some embodiments, other general design considerations aimed at enhancing pharmacokinetic (PK) properties of the nucleotide, peptide, or peptide oligonucleotide complex include one or more of the following properties: (a) building in conjugation to moieties that reduce clearance or increase cellular uptake including cholesterol or other lipids, diacylglycerol, GalNAc, palmitoyl, PEG, an RGD motif, cell penetrating peptides or moieties (e.g., a TfR-binding peptide or cell penetrating peptide as described herein). Adding cholesterol to the peptide oligonucleotide complex can improve biodistribution to the target tissue, increase cellular uptake by endocytosis, and alter the serum pharmacokinetics.

### Target Selection

The therapeutic activity and molecular method of the peptide oligonucleotide complex may depend on which target molecule (e.g., a DNA or RNA) that the nucleic acid complements, or in the case of an aptamer, which target molecule (e.g., protein or other macromolecule) it binds. Target choice can fall into one or more non-mutually exclusive categories such as tissue-target-based or disease-selective. Known targets have known mRNA and genomic sequences that can be used to design a variety of complementary nucleic acids for use in the peptide nucleotide complexes described herein depending on the activity (e.g., gene regulation, protein degradation, reduction of cancer cell activators) desired. Examples of targets are provided in TABLE 4, TABLE 5, TABLE 6, TABLE 3 and TABLE 18. For example, tissue-targeting may comprise selecting targets acting in the tissues where a TfR-binding peptide portion of the peptide oligonucleotide complex would preferentially access or accumulate. For example, serum proteins produced in the liver may be targeted, such as by a TTR to treat transthyretin-related amyloid diseases, or by various apolipoproteins to treat hypercholesterolemia or cardiovascular disease.

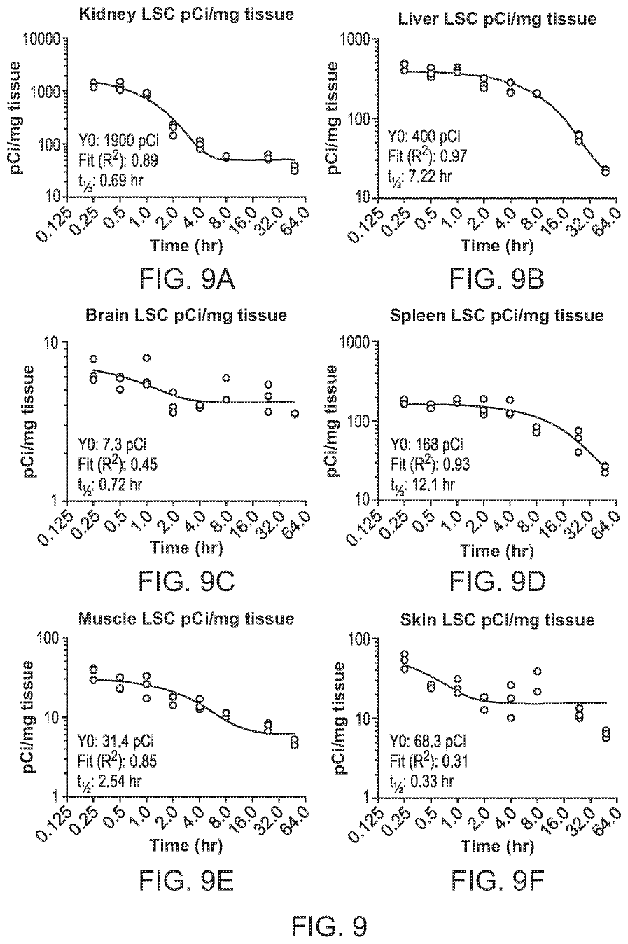

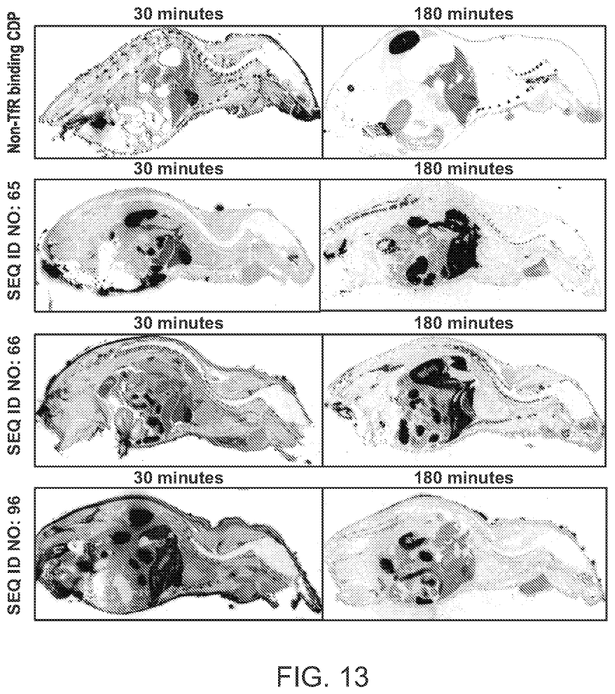

Alternatively, targets can be selected that act in areas where the TfR binding peptide accumulates (e.g., those based on WBA studies, for example FIG. 9, FIG. 12, and FIG. 13 and EXAMPLE 16), based on known expression of TfR in the tissue or target cell type, or that act where an oligonucleotide that is not complexed with the TfR binding peptide would otherwise be excluded, including such tissues as skin, bone marrow, tumors, and the CNS (brain and spinal cord). Skin diseases are often autoimmune and so molecules involved in the inflammatory pathway may be targeted by peptide oligonucleotide complexes, where preferential accumulation to skin may reduce toxicity by delivering less of the oligonucleotide to other parts of the body. Bone marrow, the site of hematopoiesis, may be targeted to correct hematopoietic diseases such as hereditary immunodeficiency, HIV (or other marrow-resident intracellular pathogens), or leukemia. Tumor targeting can be used for peptide oligonucleotide complexes of this disclosure as tumors often have high levels of TfR and are often vascularized enough for rapid perfusion of serum-resident TfR-binding peptides and their oligonucleotide cargo. Targets for the peptide oligonucleotide complexes can include oncogenes, for example by designing the nucleic acid portion of the complex to target overexpressed genes or those for which the tumor is lacking a redundant ortholog (i.e., normal cells function by using X or Y, tumors do not express Y, so X is targeted). In addition, disease-selective targeting can be used to treat conditions where the transcript is selectively found in the diseased tissue, and preferentially accumulate there, to improve safety and reduce off-target effects. For example, pathogen sequences (e.g., HIV in bone marrow or coronavirus in lung and airways) may be targeted by a peptide oligonucleotide complex to treat an infection. Furthermore, the CNS may not function as a site of active recruitment, but as a site of permissive accumulation via TfR-mediated transcytosis where other oligonucleotide delivery technology may struggle to penetrate, so transcripts with expression restricted to the CNS are amenable targets. This includes targets relevant to neurodegeneration, neuroinflammation, Alzheimer's, ALS, Huntington's Parkinson's, frontotemporal dementia, stroke, traumatic brain injury, neurodevelopmental disorders, and epilepsy (such as by modulating ion channel activity). The peptide oligonucleotide complexes of the present disclosure target liver, kidney, skin, bone marrow, CNS (e.g., brain, spinal cord), muscle, adipose, tumor, or gastrointestinal tissue.

### Diseases and Gene Targets

The nucleotide portion of the peptide oligonucleotide complexes described herein may target specific RNAs (e.g., mRNAs or pre-mRNAs) from genes expressed in cancer and other diseases. For example, the nucleotide sequence in the complex may be complementary to any target provided in TABLE 4, TABLE 5, TABLE 6, TABLE 3, or TABLE 18. The nucleotide sequence in the complex may be complementary to the target RNA, or in the case of an aptamer, may bind a target protein or other macromolecule. The a nucleotide sequence may be single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter.

In some embodiments, a target of the nucleotide in a peptide oligonucleotide complex may be a gastrointestinal target, such as a gene with pro-inflammatory, extracellular matrix-modifying, or immune cell recruitment functionality. Peptide oligonucleotide complexes described herein (e.g., a peptide oligonucleotide complex comprising a TfR-binding peptide and a nucleotide that binds a gene target mRNA) that target gastrointestinal gene targets may be used to treat various gastrointestinal disorders, including inflammatory bowel disease (IBD), ulcerative colitis, and Crohn's disease.

In some embodiments, a target of the nucleotide in a peptide oligonucleotide complex may be a central nervous system (CNS) target, such as a gene or pathways for which familial mutations are associated with a high risk of onset of neurodegenerative diseases. If disease is dominant (gain of function), the gene's transcript itself may be the target. If a disease is recessive (loss-of-function), a transcript or an ortholog may be modified to replace the lost function. For example, the gene target SMN2 may be targeted to replace loss of SMN1 function by altering splicing of SMN2 to convert the semi-functional ortholog to a fully functional replacement for SMN1. If the disease is associated with protein aggregation, clearing a portion of the target protein may be sufficient to alleviate symptoms of the disease. For example, targeting the poly-CAG (encoding poly-Q) expanded region of exon 1 of the aggregating form of Huntingtin may be used to achieve allele specificity for mutant Huntingtin over wild type Huntingtin. Peptide oligonucleotide complexes described herein (e.g., a peptide oligonucleotide complex comprising a TfR-binding peptide and a nucleotide that binds a gene target mRNA) that target CNS gene targets may be used to treat various neuronal or neurodegenerative disorders such as hereditary neurodegeneration, Huntington's disease, Alzheimer's disease, Parkinson's disease, amyotrophic lateral sclerosis (ALS), frontotemporal dementia (FTD), epilepsy, neurodevelopmental disorders, and Dravet syndrome.

In some embodiments, a target of the nucleotide in a peptide oligonucleotide complex may be a cancer target, such as a gene involved in oncogenic signaling, anti-apoptotic genes, pro-inflammatory signaling genes, protein homeostasis genes, developmental regulatory genes, or adapter protein genes that initiate downstream cell growth signaling. For example, targeting an over-expressed growth factor like HER2 can be challenging, but HER2 and other RTK (e.g., EGFR, ERBB3) signaling depends on adapter proteins like Grb2 to initiate cell growth signaling downstream. Knockdown of Grb2 can halt signaling in a way that is difficult to mutationally compensate as Grb2 loss is epistatic to HER2 activity. Cancer cells are typically under low levels of proteotoxic stress, as they are growing so quickly that their protein folding machinery struggles to keep up, so targeting protein homeostasis genes, such as heat shock proteins (HSPs), hypoxia-sensing proteins (e.g., HIF), and upregulators of the heat shock response, may reduce proteotoxic stress by helping to fold or stabilize proteins during folding. In some embodiments, a pro-inflammatory cytokine may be delivered via an mRNA in a peptide oligonucleotide complex, or an antisense construct targeting an anti-inflammatory signal may be delivered. Delivery of a pro-inflammatory signal or reduction of an anti-inflammatory signal may help to recruit B cells, T cells, macrophages, or other immune infiltrates to a tumor microenvironment. Peptide oligonucleotide complexes described herein (e.g., a peptide oligonucleotide complex comprising a TfR-binding peptide and a nucleotide that binds a gene target mRNA) that target cancer gene targets may be used to treat various cancers, including solid tumors. Developmental regulators, such as transcription factors involved in early cell fate and pluripotency, and chromatin remodeling enzymes, may be targeted to specifically harm de-differentiated cells which may be present in advanced tumors and associated with a more mobile and/or mitotic cell state. A peptide oligonucleotide construct targeting a cancer target may treat or prevent cancer by reducing oncogenic signaling, reducing target over-expression, reducing oncogenic antisense activity (e.g., miRNAs targeting tumor suppressors), and/or eliminating the source of the oncogenic signaling cascade.

Examples of gene targets (e.g., gastrointestinal, CNS, or cancer gene targets) are provided in TABLE 4.

TABLE 4
Examples of Disease-Specific Gene Targets

| Type | Target Gene | Associated Disorders |
|---|---|---|
| Gastrointestinal | Pro-inflammatory genes | IBD, Ulcerative Colitis, Crohn's Disease |
| Gastrointestinal | ECM-modifying genes | IBD, Ulcerative Colitis, Crohn's Disease |
| Gastrointestinal | Immune cell recruitment genes | IBD, Ulcerative Colitis, Crohn's Disease |
| Gastrointestinal | TNF-α | IBD, Ulcerative Colitis, Crohn's Disease |
| Gastrointestinal | ICAM-1 | IBD, Ulcerative Colitis, Crohn's Disease |
| Gastrointestinal | NF-κB | IBD, Ulcerative Colitis, Crohn's Disease |
| Gastrointestinal | NF-κB subunits | IBD, Ulcerative Colitis, Crohn's Disease |
| Gastrointestinal | NF-κB p65 subunit | IBD, Ulcerative Colitis, Crohn's Disease |
| Gastrointestinal | Smad7 | IBD, Ulcerative Colitis, Crohn's Disease |
| Gastrointestinal | Carbohydrate Sulfotransferase | IBD, Ulcerative Colitis, Crohn's Disease |
| | 15 (CHST15) | |
| Gastrointestinal | IL-23 | IBD, Ulcerative Colitis, Crohn's Disease |
| Gastrointestinal | IL-12 | IBD, Ulcerative Colitis, Crohn's Disease |
| Gastrointestinal | IL-17 | IBD, Ulcerative Colitis, Crohn's Disease |
| CNS | Poly-Q Expanded Huntingtin | Huntington's Disease |
| CNS | SOD1 | Amyotrophic Lateral Sclerosis (ALS) |
| CNS | Amyloid Precursor Protein | Alzheimer's Disease |
| | (APP) | |
| CNS | Microtubule Associated | Alzheimer's Disease, Frontotemporal |
| | Protein Tau (MAPT) | Dementia, Traumatic Brain Injury |
| CNS | SMN2 | Hereditary Neurodegeneration, |
| | | Huntington's Disease, ALS, Parkinson's |
| | | Disease, Alzheimer's Disease |
| CNS | SCN1A | Neurodevelopment Disorders, Dravet |
| | | Syndrome, Neuronal Hyperexcitability, |
| | | Epilepsy, Encephalopathy |
| CNS | ASO | Neurodevelopment Disorders, Dravet |
| | | Syndrome, Neuronal Hyperexcitability, |
| | | Epilepsy, Encephalopathy |
| CNS | SCN8A | Neurodevelopment Disorders, Dravet |
| | | Syndrome, Neuronal Hyperexcitability, |
| | | Epilepsy, Encephalopathy |
| Cancer Cells | Insulin-like Growth Factor 1 | Cancers, Solid Tumors |
| | (IGF-1) | |
| Cancer Cells | IGF-1 Receptor | Cancers, Solid Tumors |
| Cancer Cells | Androgen Receptor | Cancers, Solid Tumors |
| Cancer Cells | EGFR | Cancers, Solid Tumors |
| Cancer Cells | EGFR Isoforms | Cancers, Solid Tumors |
| Cancer Cells | EGFRvIII Isoform | Cancers, Solid Tumors |
| Cancer Cells | ERBB3 | Cancers, Solid Tumors |
| Cancer Cells | HER2 | Cancers, Solid Tumors |
| Cancer Cells | GRB2 | Cancers, Solid Tumors |
| Cancer Cells | KRAS | Cancers, Solid Tumors |
| Cancer Cells | Mutant KRAS | Cancers, Solid Tumors |
| Cancer Cells | KRAS-G12D | Cancers, Solid Tumors |
| Cancer Cells | MYC | Cancers, Solid Tumors |
| Cancer Cells | YAP1 | Cancers, Solid Tumors |
| Cancer Cells | Heat Shock Proteins | Cancers, Solid Tumors |
| Cancer Cells | HSP90 | Cancers, Solid Tumors |
| Cancer Cells | HSP70 | Cancers, Solid Tumors |
| Cancer Cells | HSP27 | Cancers, Solid Tumors |
| Cancer Cells | Hypoxia-sensing Proteins | Cancers, Solid Tumors |
| Cancer Cells | HIF1A | Cancers, Solid Tumors |
| Cancer Cells | HIF2A | Cancers, Solid Tumors |
| Cancer Cells | Pro-Inflammatory Cytokines | Cancers, Solid Tumors |
| Cancer Cells | IL-12 | Cancers, Solid Tumors |
| Cancer Cells | IL-12 mRNA | Cancers, Solid Tumors |
| Cancer Cells | TGF1B | Cancers, Solid Tumors |
| Cancer Cells | miR-21 mRNA | Cancers, Solid Tumors |
| Cancer Cells | MDM2 | Cancers, Solid Tumors |
| Cancer Cells | BCL2 | Cancers, Solid Tumors |
| Cancer Cells | FOXP3 | Cancers, Solid Tumors |
| Cancer Cells | DNMT1 | Cancers, Solid Tumors |
| Cancer Cells | HDACs | Cancers, Solid Tumors |
| Cancer Cells | Myb | Cancers, Solid Tumors |
| Cancer Cells | Fos | Cancers, Solid Tumors |
| Cancer Cells | Jun | Cancers, Solid Tumors |

Additional targets for the nucleotide portion of the peptide oligonucleotide complex herein are described in TABLE 5. The nucleic acid may complement the gene or RNA of the “Targets for downregulation” in TABLE 5. The nucleotide may be a target-binding agent be capable of binding the target or a DNA or RNA sequence encoding the target. The target-binding agent may comprise a nucleotide sequence is single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter.

TABLE 5
Targets for Downregulation

| Disease | Organ | Cell type | Target |
|---|---|---|---|
| Malaria | Bone marrow | Reticulocytes | PvRBSA |
| | | | PvRBP2b |
| | | | PfEMP1 |
| | | | pfmdr1, pfgch1 |
| Malaria | Liver | | GPX4 |
| | | | SLC7a11 |
| Parkinson's Disease | Brain | Neurons | alpha-synuclein |
| | | | TfR |
| Erythroleukemia | Bone marrow | Blasts | NUP98-KDM5A |
| | | | NTRK1 |
| | | | JAK2, K-/N-RAS |
| Acute myeloid leukemia | Bone marrow | Blasts | JAK-STAT pathway; |
| | | | Hedgehog pathway; |
| | | | PI3K/AKT pathway; |
| | | | RAF/MEK/ERK pathway; |
| | | | mTOR pathway |
| Myeloproliferative | Bone marrow | varies | JAK-STAT pathway |
| neoplasms | | | HDAC |
| | | | MDM2, LSD1, CALR |
| HIV/AIDS | Blood | Memory CD4+ | PKC, NF-κB, HSP90, HIV |
| | | T cells | Tat, TNF-α |
| HIV/AIDS | CNS | monocyte, | CCR2, CCR5; viral |
| | | macrophage | sequences; inflammatory |
| | | | pathways |
| HIV/AIDS | Bone marrow | HSC | Viral sequences such as |
| | | | TAR (tat), RRE (rev), vpr, |
| | | | U5 leader, Nef, Gag, Vif, |
| | | | Env |
| BK virus | Kidney | Uroepithelial | Viral sequences |
| | | cells | |
| MS | Varies | Varies | Viral sequences |
| Parkinson's Disease | Brain | Microglia, | Proinflammatory mediators, |
| | | astrocytes | such as IL1b, IL6, TNFa, |
| | | | IFNg, chemokines |
| Parkinson's Disease | Brain | Neurons | LRRK2 |
| Muscular disorders | Muscle | Myocytes | Myostatin |
| | Skeletal | | |
| | Cardiac | | |

Additional targets for the nucleic acid portion of the peptide oligonucleotide complex herein are described in TABLE 6. The disease and disease target can be addressed with the current oligonucleotide drug listed or with other oligonucleotides that address the same target. The nucleic acid complements the gene or mRNA of the “Target” in TABLE 6 and comprises a nucleotide sequence is single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-MIR, splice blocker ASO, or UT Adapter.

TABLE 6
Additional Gene Targets

| | | | | Exemplary | |
|---|---|---|---|---|---|
| | Exemplary | Exemplary | Binding | Admin | Exemplary |
| Disease | Oligo Drug | Modality | Target | Rout | Tissue |
| CMV retinitis | Fomivirsen | Gapmer | HCMV | IVI | Eye |
| Neovascular age- | Pegaptanib | Aptamer | VEGF-165 | IVI | Eye |
| related macular | | | | | |
| degeneration | | | | | |
| Hypercholesterolemia | Mipomersen | Gapmer | APOB | SC | Liver |
| SMA | Nusinersen | Splice | SMN2 | IT | Spinal cord |
| | | blocker | | | |
| DMD | Eteplirsen | Splice | DMD | IV | Muscle |
| | | blocker | | | |
| DMD | Golodirsen | Splice | DMD | IV | Muscle |
| | | blocker | | | |
| DMD | Viltolarsen | Splice | DMD | IV | Muscle |
| | | blocker | | | |
| DMD | Casimersen | Splice | DMD | IV | Muscle |
| | | blocker | | | |
| ADPKD | | Antagomir | miR-17 | | Kidney |
| DM1 | | ASO/siRNA | DMPK | | Muscle |
| Transthyretin- | Inotersen | Gapmer | TTR | SC | Liver |
| mediated | | | | | |
| amyloidosis | | | | | |
| Transthyretin- | Patisiran | siRNA | TTR | IV | Liver |
| mediated | | | | | |
| amyloidosis | | | | | |
| Transthyretin- | Vutrisiran | siRNA | TTR | IV | Liver |
| mediated | | | | | |
| amyloidosis | | | | | |
| Transthyretin- | Revusiran | siRNA | TTR | SC | Liver |
| mediated | | | | | |
| amyloidosis | | | | | |
| Familial | Volanesorsen | Gapmer | APOC-III | SC | Liver |
| chylomicronemia | | | | | |
| syndrome | | | | | |
| Hemophilia A + B | Fitusiran | siRNA | SERPINC1 | SC | Liver |
| Primary | Lumasiran | siRNA | HAO1 | SC | Liver, kidney |
| hyperoxaluria | | | | | |
| type 1 | | | | | |
| Acute hepatic | Givosiran | siRNA | ALAS1 | SC | Liver |
| porphyria | | | | | |
| ALS | Tofersen | Gapmer | SOD1 | IT | Spinal cord |
| ALS | | | C9orf72 | IT | Spinal cord |
| HD | Tominersen | Gapmer | HTT | IT | CNS |
| Leber's | Sepofarsen | Splice | LCA10 | IVI | Eye |
| congenital | | blocker | | | |
| amaurosis type | | | | | |
| 10 | | | | | |
| CVD | Pelacarsen | Gapmer | ApoA | SC | Liver |
| CVD | Inclisiran | siRNA | PCSK9 | SC | Liver |
| CVD | | | APOC-III | | |
| UC | Alicaforsen | Gapmer | ICAM-1 | IV | GI |
| AD | | Gapmer | MAPT | IT | CNS |
| Clotting disorder | | | Factor XI | | |
| T2D | | | GCGR | | |
| NASH | | | DGAT2 | | Liver |
| Cancer | Apatorsen | Gapmer | HSP27 | IV | Various solid |
| | | | | | tumors |
| Cancer | | | STAT3 | | |
| Cancer | | | AR | | |
| Cancer | Cobomarsen | Antagomir | miR-155 | | |
| Cancer | Prexigebersen | Translation | GRB2 | IV | AML |
| | | blocker | | | |
| Cancer | Trabedersen | Gapmer | TGFB2 | | Glioma, |
| | | | | | prostate |
| | | | | | cancer |
| MS, DMD | | | CD49d | | |
| Acromegaly | | | GHR | | |
| HBV | | | HBV surface | | |
| | | | antigen | | |
| Centronuclear | | | DNM2 | | |
| myopathy | | | | | |
| CF | | | ENAC | Nebulized | |
| Dyslipidemia | | | ANGPTL3 | | |
| Hypertension | | | AGT | | |
| Primary IgA | | | Factor B | | |
| nephropathy | | | | | |
| HAE | | | Kallikrein | | |
| | | | B1 | | |
| Retinitis | | | RHO | | |
| pigmentosa | | | | | |
| Retinitis | | | USH2A | | |
| pigmentosa | | | | | |
| Dystrophic | | | COL7A1 | | |
| epidermolysis | | | | | |
| bullosa | | | | | |
| Alport syndrome | | Antagomir | miR-21 | | |
| PD | | | LRRK2 | IT | |
| MSA | | | SNCA | IT | |
| Psoriasis | | | IL17RA | Topical | |

Any targets for the nucleic acid portion of the peptide oligonucleotide complex described herein can be used in conjunction with a U1 adapter to degrade targeted mRNAs. The target recognition (or complementary nucleic acid to the target mRNA) portion directs the peptide oligonucleotide complex to the targeted mRNA selected for degradation, while the U1 portion prevents the addition of polyA to the mRNA resulting in degradation of the targeted mRNA. U1 adapters can comprise any nucleotide sequence complementary to the ssRNA component of the U1 small nuclear ribonucleoprotein (U1 snRNP). In some embodiments, the U1 adapter sequences engage the U1 snRNP near its poly A site. In some embodiments, the length of the U1 adapter is 15 to 25 nt in length, or about 20 nt in length. In some embodiments, the U1 adapter is above 40% in its G/C content. Exemplary U1 adapters are shown in TABLE 7, in conjunction with a target nucleic acid “target recognition” portion which comprises a nucleotide sequence is single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, or splice blocker ASO. The 10-19 nt U1 Adapter is italicized.

TABLE 7
Examples of Target Recognition Constructs with U1 Adapters
5′-Nucleic Acid complementary to Target mRNA-U1 Adapter-3′

5′-[Target_recognition]UCCCCUGCCAGGUAAGUAU-3′ [19 nt]
5′-[Target_recognition]CCCUGCCAGGUAAGUAU-3′ [17 nt]
5′-[Target_recognition]CUGCCAGGUAAGUAU-3′ [15 nt]
5′-[Target_recognition]UGCCAGGUAAGUAU-3′ [14 nt]
5′-[Target_recognition]GCCAGGUAAGUAU-3′ [13 nt]
5′-[Target_recognition]CCAGGUAAGUAU-3′ [12 nt]
5′-[Target_recognition]CAGGUAAGUAU-3′ [11 nt]
5′-[Target_recognition]CAGGUAAGUA-3′ [10 nt]

Exemplary U1 adapters include:
(SEQ ID NO: 364)
UCCCCUGCCAGGUAAGUAU;
(SEQ ID NO: 365)
CCCUGCCAGGUAAGUAU;
(SEQ ID NO: 366)
CUGCCAGGUAAGUAU;
(SEQ ID NO: 367)
UGCCAGGUAAGUAU;
(SEQ ID NO: 368)
GCCAGGUAAGUAU;
(SEQ ID NO: 369)
CCAGGUAAGUAU;
(SEQ ID NO: 370)
CAGGUAAGUAU;
and
(SEQ ID NO: 371)
CAGGUAAGUA

The target-binding agent (e.g., a nucleotide of a peptide oligonucleotide complex) may be capable of binding the targets described in TABLE 4, TABLE 5, or TABLE 6, TABLE 3, or TABLE 18, or a DNA or RNA molecule encoding the targets described in TABLE 4, TABLE 5, or TABLE 6, TABLE 3, or TABLE 18. It is understood that any oligonucleotide may be used that is complementary to a portion of the target DNA or RNA molecule. Such target binding agent may comprise a nucleotide sequence is single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter. Such oligos may be about 5 to 30 nt in length, 10 to 25 nt in length, 15 to 25 nt in length, 19 to 23 nt in length, or at least 10 nt in length, at least 15 nt in length, at least 20 nt in length, at least 25 nt in length, or at least 30 nt in length, at least 50 nt in length, at least 100 nucleotides in length across any portion of the target RNA. Examples of sequences to which such oligonucleotides may bind (e.g., are complementary to) include SEQ ID NO: 395-SEQ ID NO: 428, provided in TABLE 3, or any genomic or ORF sequence referenced in TABLE 18. One of skill in the art can readily design or determine the length of the target binding agent and whether the target binding agent is complementary to the reference target RNA sequence, and can thus determine using the chemistry of RNA and DNA where such target binding agent will bind to such reference target RNA sequence for the designed length across any portion of the target RNA. Consequently, for any RNA target described herein, including for any of the targets or molecules encoding the targets described in TABLE 4, TABLE 5, and TABLE 6, and SEQ ID NO: 395-SEQ ID NO: 428, provided in TABLE 3, or any genomic or ORF sequence referenced in TABLE 18 such target binding agent of any nt length is described.

In some embodiments, a nucleotide binds to the target molecule with a melting temperature of not less than 37° C. and not more than 99° C. In some embodiments, a nucleotide binds to the target molecule with a melting temperature of not less than 40° C. and not more than 85° C., not less than 40° C. and not more than 65° C., not less than 40° C. and not more than 55° C., not less than 50° C. and not more than 85° C., not less than 60° C. and not more than 85° C., or not less than 55° C. and not more than 65° C.

In some embodiments, a nucleotide binds the target molecule with an affinity of not more than 500 nM, not more than 100 nM, not more than 50 nM, not more than 10 nM, not more than 1 nM, not more than 500 pM, not more than 400 pM, not more than 300 pM, not more than 200 pM, or not more than 100 pM. In some embodiments, a nucleotide binds the target molecule with an affinity of not more than 500 nM and not less than 100 pM, not more than 100 nM and not less than 200 pM, not more than 50 nM and not less than 300 pM, not more than 10 nM and not less than 400 pM, or not more than 1 nM and not less than 500 pM.

In some embodiments, a nucleotide comprises at least 90%, at least 92%, at least 93%, at least 94%, at least 95%, at least 96%, at least 97%, at least 98%, or at least 99% sequence identity to any one of SEQ ID NO: 364-SEQ ID NO: 394. In some embodiments, a nucleotide comprises a sequence of any one of SEQ ID NO: 364-SEQ ID NO: 394, any one of SEQ ID NO: 364-SEQ ID NO: 394 wherein U is replaced with T, or any one of SEQ ID NO: 364-SEQ ID NO: 394 wherein T is replaced with U. In some embodiments, a nucleotide comprises no more than 1, 2, 3, 4, or 5 base changes relative to a sequence of any one of SEQ ID NO: 364-SEQ ID NO: 394.

In some embodiments, a nucleotide is at least 60%, at least 70%, at least 80%, at least 90%, at least 92%, at least 93%, at least 94%, at least 95%, at least 96%, at least 97%, at least 98%, at least 99%, or 100% reverse complementary to the target molecule. In some embodiments, a nucleotide is 100% reverse complementary to the target molecule. In some embodiments, a nucleotide comprises no more than 1, 2, 3, 4, or 5 base pair mismatches upon binding to the target molecule. In some embodiments, a nucleotide comprises at least 1, 2, 3, 4, or 5 base pair mismatches upon binding to the target molecule.

In some embodiments, a nucleotide may modulate an activity of a target molecule. In some embodiments, modulating the activity of the target molecule comprises reducing expression of the target molecule, increasing the expression of the target molecule, reducing translation of the target molecule, degrading the target molecule, reducing a level of the target molecule, modifying the processing of the target molecule, modifying the splicing of the target molecule, inhibiting processing of the target molecule, reducing a level of a protein encoded by the target molecule, or blocking an interaction with the target molecule. In some embodiments, the expression of the target molecule is reduced by at least 10%, 25%, 30%, 40%, 50%, 60%, 75%, 80%, 90%, 95%, 99%, 99.5%, or 99.9%. In some embodiments, the translation of the target molecule is reduced by at least 10%, 25%, 30%, 40%, 50%, 60%, 75%, 80%, 90%, 95%, 99%, 99.5%, or 99.9%. In some embodiments, the expression of the target molecule is reduced by a factor of at least 2, 4, 8, 10, 15, 16, 20, 32, 50, 64, 100, 128, 200, 256, 500, 512, or 1000. In some embodiments, the translation of the target molecule is reduced by a factor of at least 2, 4, 8, 10, 15, 16, 20, 32, 50, 64, 100, 128, 200, 256, 500, 512, or 1000. In some embodiments, at least 10%, 25%, 30%, 40%, 50%, 60%, 75%, 80%, 90%, 95%, 99%, 99.5%, or 99.9% of the target molecule is degraded. In some embodiments, the level of the protein encoded by the target molecule is reduced by at least 10%, 25%, 30%, 40%, 50%, 60%, 75%, 80%, 90%, 95%, 99%, 99.5%, or 99.9%. In some embodiments, modifying the splicing of the target molecule increases a level of a protein encoded by the target molecule by at least 10%, 25%, 30%, 40%, 50%, 60%, 75%, 80%, 90%, 95%, 99%, 99.5%, or 99.9%.

### Nucleotides

A peptide oligonucleotide complex of the present disclosure may comprise a nucleotide complexed with a protein (e.g., a TfR-binding peptide). The nucleotide may comprise single stranded DNA, single stranded RNA, double stranded DNA, double stranded RNA, or combinations thereof. In some embodiments, a nucleotide of a peptide oligonucleotide complex may be non-naturally occurring, also referred to as an “engineered nucleotide”. In some embodiments, a nucleotide may comprise a naturally occurring sequence. A nucleotide may be exogenously expressed, enzymatically synthesized in vitro, or chemically synthesized. For example, a nucleotide may be expressed in a bacterial, yeast, or mammalian cell line and purified for use in a peptide oligonucleotide complex of the present disclosure. In another example, a nucleotide may be enzymatically synthesized in vitro using an RNA or DNA polymerase. In another example, a nucleotide may be chemically synthesized on a solid support using protected nucleotides.

One example of a chemical synthesis method that may be used to prepare a nucleotide for use in a peptide oligonucleotide complex of the present disclosure is phosphoramidite synthesis. Briefly, single nucleotide residues may be sequentially added from 3′ to 5′ to the growing nucleotide chain by repeating the steps of de-blocking (detritylation), coupling, capping, and oxidation. Phosphoramidite synthesis may be performed on a solid support such as controlled pore glass (CPG) or macroporous polystyrene (MPPS). Similarly, thiophosphorodiamidate may be used.

A nucleotide of a peptide oligonucleotide complex may bind to a target molecule (e.g., a target DNA, a target RNA, or a target protein). In some embodiments, binding of the oligonucleotide to the target molecule may alter an activity of the target molecule. For example, binding of an oligonucleotide (e.g., an siRNA, an miRNA, a gapmer, or a U1 adaptor) to a target mRNA or pre-mRNA may increase or decrease translation of the target mRNA or pre-mRNA. In another example, binding of a nucleotide to a target DNA may increase or decrease expression of a gene encoded by the target DNA. In another example, binding of a nucleotide to an RNA (such as a transcript, pre-RNA, unspliced RNA, nuclear RNA, complimentary sequence to a NAT, or mRNA) expressed from a target DNA such as a gene or ORF may increase or decrease expression of a gene encoded by the target DNA. In another example, binding of an oligonucleotide (e.g., an aptamer) to a target protein may increase or decrease activity (e.g., an enzymatic activity or a binding activity) of the target protein. In some embodiments, the target molecule may be associated with a disease or condition and increasing or decreasing the activity of the target molecule may treat the disease or condition.

A sequence of the oligonucleotide of a peptide oligonucleotide complex may be selected for its ability to bind to or modulate the activity of a target molecule. In some embodiments, an oligonucleotide may be reverse complementary to a target DNA or RNA molecule. For example, an siRNA oligonucleotide may be reverse complementary to a target RNA molecule. In some embodiments, am oligonucleotide may be partially reverse complementary (e.g., comprising one or more mis-matched base pairs) to a target DNA or RNA molecule. For example, an siRNA oligonucleotide may comprise a base mismatch relative to a target RNA molecule. In some embodiments, a sequence of the oligonucleotide may be selected for its annealing temperature relative to a target DNA or RNA molecule. A preferred annealing temperature may be achieved by selecting the length of the nucleotide, the degree of complementarity of the nucleotide to the target molecule, the chemistry of the nucleotides, or any combination thereof. Nucleotide sequence parameters (e.g., complementarity, annealing temperature, melting temperature, base mismatches, and binding affinity) may be calculated using any available software, such as ITD OligoAnalyzer and the like. In some embodiments, an oligonucleotide may adopt a secondary structure that binds to a target DNA, RNA, or protein molecule. For example, an aptamer may adopt a secondary structure to bind to a target protein. The aptamer sequence may be selected to adopt a secondary structure that binds to a target protein. Nucleotide secondary structure may be predicted using any available software, such as RNAfold and the like. In some embodiments, a nucleotide sequence may be determined experimentally by selecting for the ability to bind to a target molecule. For example, a nucleotide library may be contacted to a target molecule, and sequences that bind to the target molecule may be identified.

In some embodiments, a nucleotide comprises a G/C content of not less than 20% and not more than 80%. In some embodiments, a nucleotide comprises a G/C content of not less than 30% and not more than 65%. In some embodiments, the nucleotide comprises a G/C content of not less than 20%, not less than 25%, not less than 30%, not less than 35%, not less than 40%, not less than 45%, or not less than 50%. In some embodiments, the nucleotide comprises a G/C content of not more than 80%, not more than 75%, not more than 70%, not more than 65%, or not more than 50%. In some embodiments, a nucleotide comprises an A/T content or A/U content of not less than 20% and not more than 80%. In some embodiments, a nucleotide comprises an A/T content or A/U content of not less than 30% and not more than 65%. In some embodiments, the nucleotide comprises a A/U (or A/T, or combination of A/U and A/T) content of not less than 20%, not less than 25%, not less than 30%, not less than 35%, not less than 40%, not less than 45%, or not less than 50%. In some embodiments, the nucleotide comprises a A/U content (or A/T, or combination of A/U and A/T) of not more than 80%, not more than 75%, not more than 70%, not more than 65%, or not more than 50%. In some embodiments, a nucleotide has a length of no more than 1000 nt, 600 nt, 200 nt, 100 nt, 60 nt, 56 nt, 52 nt, 50 nt, 48 nt, 46 nt, 44 nt, 22 nt, 40 nt, 38 nt, 36, nt, 34 nt, 32 nt, 30 nt, or 24 nt. In some embodiments, a nucleotide has a length of from 24 to 100 nt, from 24 to 60 nt, from 24 to 50 nt, or from 36 to 50 nt. In some embodiments, a nucleotide has a length of about 42 nt.

In some embodiments, a nucleotide has a length of no more than 500 nt, 300 nt, 100 nt, 50 nt, 30 nt, 28 nt, 26 nt, 25 nt, 24 nt, 23 nt, 22 nt, 21 nt, 20 nt, 19 nt, 18, nt, 17 nt, 16 nt, 15 nt, or 12 nt. In some embodiments, a nucleotide has a length of from 12 to 50 nt, from 12 to 30 nt, from 12 to 25 nt, from 18 to 25 nt, from 18 to 25 nt, from 19 to 23 nt, or from 20 to 22 nt. In some embodiments, a nucleotide has a length of about 21 nt.

### Nucleotide Modifications

In some embodiments, the nucleic acid portion of the peptide oligonucleotide complexes described herein contain one or more bases within the nucleic acid molecule that are modified. Such modifications can occur whether the nucleic acid portion a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter. One or more bases in a given nucleotide sequence may be modified to increase in vivo stability, to increase resistance to enzymes such as nucleases, increase protein binding including to serum proteins, increase in vivo half-life, to modify the tissue biodistribution, or to modify how the immune system responds. The phosphonate, the ribose, or the base may be modified. In some aspects, the modification comprises a phosphorothioate modification, a phosphodiester modification, a thio-phosphoramidate modification, a methyl phosphonate modification, a phosphorodithioate modification, a methoxypropylphosphonate modification, a 5′-(E)-vinylphosphonate modification, a 5′methyl phosphonate modification, an (S)-5′-C-methyl with phosphate modification, a 5′-phosphorothioate modification, a peptide nucleic acid (PNA), a 2′-O methyl modification, a 2′-O-methoxyethyl (2′-O-Me) modification, a 2′-fluoro (2′-F) modification, a 2′-deoxy-2′-fluoro modification, a 2′arabino-fluor modification, a 2′-O-benyzl modification, a 2′-O-methyl-4-pyridine modification, a locked nucleic acid (LNA), an amino-LNA, a thio-LNA, an ENA, an amino ENA, a carbo-ENA, a (S)-cEt-bridged nucleic acid, an (S)-MOE, a bridged nucleic acid, a tricyclo-DNA, a morpholino nucleic acid (PMO), an unlocked nucleic acid (UNA), a glycol nucleic acid (GNA), a bridged nucleic acid (BNA), an ethyl (S)-cEt nucleic acid, a pseudouridine, a 2′-thiouridine, an N6′methyadenosine, a 5′-methylcytidine, a 5′-fluoro-2′-deoxyuridine, a N′ethylpiperidine 7′-EAA triazole modified adenine, an N-ethylpiperidine 6′-triazole modified adenine, a 6′-phenylpyrrolocytosine, a 2′,4′-difluorotoluyl ribonucleoside, a 5′nitroindole, a 5′ methyl, a 5′ phosphonate, an inverted A base, a 2′-H (deoxyribose), a 2′-OH (ribose), or any combination thereof. The oligonucleotide may be comprised entirely of a combination of 2′-O-Me and 2′-F modifications. Diastereomers or one or both stereoisomers may be used. Any of the stabilization chemistries or patterns, including STC, ESC, advanced, ESC, AD1-3, AD5, disclosed in Hu Signal Transduction and Targeted Therapy 2020, 5:101 can be used. Pyrimidines can be 2′-fluoro-modified, which can increase stability to nucleases but can also increase immune system activation. The RNA backbone can be phosphorothioate-substituted (where the non-bridging oxygen is replaced with sulfur), which can increase resistance to nuclease digestion as well as altering the biodistribution and tissue retention and increasing the pharmacokinetics such as by increasing protein binding, but can also induce more immune stimulation. Methyl phosphonate modification of an RNA can also be used. 2′-Omethyl and 2′-F RNA bases can be used, which can protect against base hydrolysis and nucleases and increase the melting temperature of duplexes. Bridged, Locked, and other similar forms of Bridged Nucleic Acids (BNA, LNA, cEt) where any chemical bridge such as an N—O linkage between the 2′ oxygen and 4′ carbons in ribose can be incorporated to increase resistance to exo- and endonucleases and enhance biostability. These include BNA where an N—O linkage between the 2′ and 4′ carbons occur and where any chemical modification of the nitrogen (including but not limited to N—H, N—CH3, N-benzene) in the bridge can be added to increase stability RNA backbone or base modifications can be placed anywhere in the RNA sequence, at one, multiple, or all base locations. Optionally, phosophorothioate nucleic acid linkages may be used between the 2-4 terminal nucleic acids of one or both sequences. Optionally 2′F modified nucleic acids may be used at least at 2-4 positions, at least 5%, at least 10% at least 25% of internal positions, at least 50%, at least 75%, or up to 100% of internal positions, all internal positions or all positions. Optionally, one or more of 2′F base, an LNA base, a BNA base, an ENA base, a 2′O-MOE base, a morpholino base, a 2′OMe base, a 5′-Me base, a (S)-cEt base or combinations thereof may be used at least at 2-4 positions, at least 5%, at least 10% at least 25% of internal positions, at least 50%, at least 75%, or up to 100% of internal positions, all internal positions or all positions.

Modified bases can be used to increase in the in vivo half-life of the oligonucleotide. They can allow the oligonucleotide to remaining intact in the serum, endosome, cytosol, or nucleus, including for days, weeks, or months. This can allow ongoing activity, including if the oligonucleotide is slowly released from the endosome over days, weeks, or months within a given cell (such as described in Brown et al., Nucleic Acids Research, 2020, p11827-11844).

In some embodiments, a nucleotide comprises at least one phosphorothioate linkage. In some embodiments, a peptide oligonucleotide complex comprises from 1 to 12 phosphorothioate linkages. In some embodiments, a nucleotide comprises at least one thiophosphoroamidate linkage. In some embodiments, a nucleotide comprises from 1 to 12 thiophosphoroamidate linkages. In some embodiments, a nucleotide comprises at least one modified base. In some embodiments, at least modified base comprises a 2′F base, an LNA base, a BNA base, an ENA base, a 2′O-MOE base, a 5′-Me base, a (S)-cEt base, a 2′OMe base, a morpholino base, or combinations thereof.

Peptide Oligonucleotide Complexes with Additional Active Agents

A peptide oligonucleotide complex of the present disclosure (e.g., a peptide oligonucleotide complex comprising a TfR-binding peptide and a nucleotide) may be further conjugated, linked, or fused to an active agent in addition to the nucleotide active agent (e.g., a target-binding agent capable of binding a target molecule). Such additional active agent may be complexed, fused, linked or conjugated to one or more of the peptide, nucleotide, or linker within the peptide oligonucleotide complex. In some embodiments, the active agent may be directly or indirectly linked to the peptide of the peptide oligonucleotide complex or the nucleotide of the peptide oligonucleotide complex. A peptide nucleic acid complex further comprising an additional active agent may be referred to as a peptide-active agent conjugate or a peptide construct.

The peptide oligonucleotide complexes of the present disclosure can also be used to deliver another active agent. Peptides according to the present disclosure can be conjugated to, linked to, or fused to an agent for use in the treatment of tumors and cancers or other diseases. For example, in certain embodiments, the peptides described herein are fused or conjugated to another molecule, such as an active agent that provides an additional functional capability. A peptide or nucleotide can be fused with an active agent through expression of a vector containing the sequence of the peptide with the sequence of the active agent. In various embodiments, the sequence of the peptide and the sequence of the active agent can be expressed from the same Open Reading Frame (ORF). In various embodiments, the sequence of the peptide and the sequence of the active agent can comprise a contiguous sequence. The peptide and the active agent can each retain similar functional capabilities in the peptide construct compared with their functional capabilities when expressed separately. In certain embodiments, examples of active agents can include other peptides.

In certain embodiments, examples of active agents include other peptides such as neurotensin peptide. Neurotensin is a 13 amino acid neuropeptide that can be involved in the regulation of luteinizing hormone and prolactin release, and can interact with the dopaminergic system, but does not cross the blood brain barrier and is also rapidly metabolized by peptidases (Wang, et al., Curr. Pharm. Des. 21(7); 840-848 (2015)).

Neurotensin is a 13-amino acid peptide found in the central nervous system and the gastrointestinal tract and plays a role in a wide range of physiologic and pathologic processes. Various activities of neurotensin and potential therapeutic roles of neurotensin agonists are described in the following: Mustain, Rychahou, and Evers, Curr Opin Endocrinol Diabetes Obes (2011) and Boules, Li, Smith, Fredrickson, and Richelson, Front Endocrinol (Lausanne) (2013). NT plays a role in gut motility, modulation of the cardiovascular system, naloxone-independent antinociception, hypothermia, controls of anterior pituitary hormone secretion, muscle relaxation, central blood pressure, and inflammation. NT mediates its effects through three receptors NTS1, NTS2, an NTS3. NTS1 is a high affinity receptor and is expressed broadly throughout the CNS, including medial septal nucleus, nucleus basalis magnocellularis, suprachiasmatic nucleus, SN, and VTA, small dorsal root ganglion neurons of the spinal cord, in both neurons and glial cells. NTS2 is localized mainly in the olfactory system, the cerebral and cerebellar cortices, the hippocampal formation, and selective hypothalamic nuclei, VTA, and SN. NTS3 is also expressed at various locations throughout the brain. NT is involved in modulating dopamine neurotransmission, may play a role in the serotonergic system including antinociception, sleep-wake cycle, and stress, a role in glutamate release and may have antipsychotic effects. NT is involved in neuroendocrine regulation, including paracrine and autocrine roles, CRH, GnRH, GHRH, prolactin, ACTH, gonadotropic hormones, growth hormones, prolactin, thyroid hormone modulation, as well as gut motility, intestinal inflammation, lipid metabolism, appetite, and food intake. NT has been implicated in the pathophysiology of several CNS disorders such as schizophrenia, drug abuse, Parkinson's disease (PD), pain, central control of blood pressure, eating disorders, stroke, Alzheimer's, as well as, cancer and inflammation and other disorders of endocrine function. Disruption in the normal mechanisms can lead to diseases ranging from schizophrenia to colorectal cancer. By selective targeting or blockade of specific neurotensin receptors, investigators have identified potential drugs for use in the treatment of schizophrenia, alcoholism, chronic pain, or cancer. NT can provide opioid-independent analgesia and can treat thermal, visceral, and persistent inflammatory pain, somatic pain and visceral pain, and plays a role in stress-induced antinociception. NT exerts a mu-opioid-independent, anti-nociceptive effects. NT agonists can block effects of psychostimulants such as cocaine and amphetamine and nicotine. Plasma NT levels are higher in Parkinson's disease patients, and administration of NT can reduce muscular rigidity and tremors. NT can also reduce body temperature and reduce blood pressure.

TABLE 8 discloses exemplary neurotensin (NT) peptide variants (Boules, et al., Diverse roles of neurotensin agonists in the central nervous system, Frontiers in Endocrinology, 2013). Any peptides of the present disclosure (e.g., any one of SEQ ID NO: 1-SEQ ID NO: 134 or SEQ ID NO: 306-SEQ ID NO: 335) can be fused or otherwise conjugated to any of the NT peptide variants disclosed herein (e.g., any of the NT peptide variants disclosed in TABLE 8), or fragments thereof. In some embodiments, the peptides of the disclosure are fused or otherwise conjugated a neurotensin (NT) peptide variant comprising a at least 53%, at least 60%, at least 69%, at least 75%, at least 77%, at least 85%, least 90%, at least 92% sequence identity with SEQ ID NO: 341. In other embodiments, the peptides of the disclosure are fused or otherwise conjugated a neurotensin (NT) peptide variant comprising the sequence ELYENKP (SEQ ID NO: 355) or ELYENKP-X₁-X₂-P-X₃-X₄-L (SEQ ID NO: 356), wherein X₁ and X₂ can be Lys or Arg, X₃ can be Trp or Tyr, and X₄ can be Ile or Leu, or wherein none, or one or more of X₁-X₄ and Leu comprises a modified amino acid residue or non-natural amino acid residue.

TABLE 8
Neurotensin Peptide Variants

| SEQ ID NO | Amino Acid Sequence |
|---|---|
| SEQ ID NO: 341 | ELYENKPRRPYIL |
| SEQ ID NO: 350 | ELYENKP-[N-methyl-Arg]-KP-[l-neo-Trp]-[tert-Leu]-L |
| SEQ ID NO: 351 | ELYENKPR-[d-Lys]-P-[l-neo-Trp]-[tert-Ile]-L |
| SEQ ID NO: 352 | ELYENKPR-[d-Orn]-P-[l-neo-Trp]-[tert-Leu]-L |
| SEQ ID NO: 353 | ELYENKP-[N-methyl-Arg]-RP-[d-3,1-Nal]-[tert-Leu]-L |
| SEQ ID NO: 354 | ELYENKP-[H-Lys-psi\|CH2NH]]-KPY-[Tle]-[Leu-Oet] |
| SEQ ID NO: 355 | ELYENKP |
| SEQ ID NO: 356 | ELYENKPX₁X₂PX₃X₄L |

Therefore, the fusion of neurotensin peptide and one of the peptides described herein that can cross the blood brain barrier can produce a fusion peptide capable of crossing the blood barrier which can retain the functional capabilities of neurotensin peptide. For example, the DNA sequence of a peptide of the present disclosure is inserted into the gene of neurotensin to manufacture peptide-neurotensin fusions. In some embodiments, the peptides of this disclosure are fused to neurotensin or a functional fragment thereof. A functional fragment of neurotensin can be at least 5 amino acid residues, at least 6 amino acid residues, at least 7 amino acid residues, at least 8 amino acid residues, at least 9 amino acid residues, at least 10 amino acid residues, at least 11 amino acid residues, at least 12 amino acid residues, or at least 13 amino acid residues long.

As another example, in certain embodiments, the peptides or nucleotides described herein are attached to another molecule, such as an active agent that provides a functional capability. In some embodiments, an active agent is an immunotherapeutic agent, a CTLA-4 targeting agent, a PD-1 targeting agent, a PDL-1 targeting agent, an IL15 agent, a fused IL-15/IL-15Ra complex agent, an IFNgamma agent, an anti-CD3 agent, an ion channel modulator, a Kᵥ1.3 inhibitor, an auristatin, MMAE, a maytansinoid, DM1, DM4, doxorubicin, a calicheamicin, a platinum compound, cisplatin, a taxane, paclitaxel, SN-38, a BACE inhibitor, a Bcl-xL inhibitor, WEHI-539, venetoclax, ABT-199, navitoclax, AT-101, obatoclax, a pyrrolobenzodiazepine or pyrrolobenzodiazepine dimer, a dolastatin, or a neurotransmitter such as neurotensin.

In some embodiments, TfR-binding peptides can direct the active agent (e.g., a target-binding nucleotide, small molecule, peptide, or protein active agent) into the cell. In further embodiments, TfR-binding peptides can direct the active agent into the nucleus. In some embodiments, the active agent has intrinsic tumor-homing properties, or the active agent can be engineering to have tumor-homing properties. In some embodiments, 1, 2, 3, 4, 5, 6, 7, 8, 9, or 10 active agents can be linked to a peptide or nucleotide. Multiple active agents (e.g., multiple target-binding nucleotides) can be attached by methods such as conjugating to multiple lysine residues and/or the N-terminus, or by linking the multiple active agents to a scaffold, such as a polymer or dendrimer and then attaching that agent-scaffold to the peptide (such as described in Yurkovetskiy, A. V., Cancer Res 75(16): 3365-72 (2015)). Examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an oligonucleotide, antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter an antibody, a single chain variable fragment (scFv), an antibody fragment, an aptamer, a cytokine, an interferon, a hormone, an enzyme, a growth factor, a checkpoint inhibitor, a PD-1 inhibitor, a PD-L1 inhibitor, a CD47 inhibitor, a CTLA4 inhibitor, a CD antigen, a chemokine, a neurotransmitter, an ion channel inhibitor, an ion channel activator, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, a complement fixing peptide or protein, a tumor necrosis factor inhibitor, a tumor necrosis factor activator, a tumor necrosis factor receptor family agonist, a tumor necrosis receptor antagonist, a Tim-3 inhibitor, a protease inhibitor, an amino sugar, a chemotherapeutic, a cytotoxic molecule, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, polyethylene glycol, a lipid, a dendrimer, a fatty acid, or an Fc region, or an active fragment or a modification thereof.

In some embodiments, the peptide or nucleotide is covalently or non-covalently linked to an active agent, e.g., directly or via a linker. For example, cytotoxic molecules that can be used include auristatins, MMAE, MMAF, dolostatin, auristatin F, monomethylaurstatin D, DM1, DM4, maytansinoids, maytansine, calicheamicins, N-acetyl-γ-calicheamicin, pyrrolobenzodiazepines, PBD dimers, doxorubicin, vinca alkaloids (4-deacetylvinblastine), duocarmycins, cyclic octapeptide analogs of mushroom amatoxins, epothilones, and anthracylines, CC-1065, taxanes, paclitaxel, cabazitaxel, docetaxel, SN-38, irinotecan, vincristine, vinblastine, platinum compounds, cisplatin, methotrexate, BACE (beta-secretase 1) inhibitors such as verubecestat, chlorambucil, mitomycin C. Additional examples of active agents are described in McCombs, J. R., AAPS J, 17(2): 339-51 (2015), Ducry, L., Antibody Drug Conjugates (2013), and Singh, S. K., Pharm Res. 32(11): 3541-3571 (2015). Additional examples of therapeutic payloads which therapeutic efficacy can be significantly improved when used in combination with the compositions and methods of the present disclosure include Carmustine, Cisplatin, Cyclophosphamide, Etoposide, Irinotecan, Lomustine, Procarbazine, Temozolomide, Vincristine, and Bevacizumab. Additional examples of therapeutic payloads are compounds that have therapeutic benefit in neurodegenerative diseases such as BACE inhibitors or auto-immunity diseases, and effective treatments for pain and migraine such as CGRP Receptor antagonists. Active agents used to treat pain are also consistent with the present disclosure. For example, in some embodiments, the active agent is a neurotransmitter, such as neurotensin. Thus, peptide constructs disclosed herein are peptide-neurotensin constructs. Exemplary linkers suitable for use with the embodiments herein are discussed in further detail below.

Only a small fraction of currently available drug molecules have applicability in CNS diseases due to their poor BBB penetration capabilities. About 98% of small molecule drugs do not or do only to a very limited degree cross the BBB. In addition, nearly 100% of macromolecular drug molecules (e.g., antibodies) do not exhibit significant BBB penetration capabilities. (See e.g., Mikitsh et al. Pathways for Small Molecule Delivery to the Central Nervous System Across the Blood-Brain Barrier, Perspect Medicin Chem. 2014; 6: 11-24). Thus, drug molecules available or currently in preclinical or clinical development can be used in combination with the presently described methods and compositions to provide improved therapeutic activity of these drugs in the cells, tissues, or organs expressing TfR (e.g., cancer cells or immune cells) and thus provide superior clinical outcomes compared to conventional therapies. For example, a drug molecule that shows activity against a specific biological target (e.g., a protein or a nucleic acid) but is prevented from reaching said target in vivo due to cellular barriers or cell membranes, said drug molecule can be conjugated to, linked to, or fused to a TfR-binding peptide of the present disclosure for enhanced delivery via TfR-mediated transport across those cellular barriers (e.g., BBB) or cell membranes (e.g., a cell membrane of a cancer cell). The peptide-drug conjugate or fusion molecule can cross the BBB via TfR-mediated transcytosis and thus provide significantly higher drug concentrations (e.g., therapeutic concentrations in the CNS to treat or prevent a CNS disorder (e.g., a brain cancer). For example, palbociclib has been shown efficacy in the treatment of breast cancer, but activity against CNS tumors has been limited. Thus, conjugating the drug palbociclib to a TfR-binding peptide of the present disclosure can enable its use in the treatment and/or prevention of tumors that are not accessible for the drug molecule itself, for instance those located in the CNS (e.g., brain tumors). Brain tumors that can be treated or prevented using conjugates or fusion molecules comprising one or more TfR-binding peptides of the present disclosure can include glioblastoma, astrocytoma, glioma, medulloblastoma, ependymoma, choroid plexus carcinoma, midline glioma, and diffuse intrinsic pontine glioma.

In some embodiments, the TfR-binding peptides and peptide oligonucleotide complexes as described herein can be used to treat and/or prevent diseases in non-CNS and/or peripheral cells, tissues, or organs. For example, the TfR-binding peptides as described herein can be used to treat and/or prevent muscle diseases. Drug or drug candidates that may be used in combination with the TfR-binding peptides to treat or prevent muscle diseases can include drugs or drug candidates for muscular dystrophy, spinal bulbar muscular dystrophy (SBMA), myotonic dystrophy, cachexia, or sarcopenia. In some embodiments, drugs can be senolytics or other anti-aging molecules (e.g., caspases), or gene therapies for diseases of the muscle.

In some embodiments, the TfR-binding peptides as described herein can be used to treat pain. The TfR binding peptides can be linked to neurotensin (NT) to generate CDP-NT peptide constructs (e.g., SEQ ID NO: 135-SEQ ID NO: 140). These peptide constructs can target neurotensin receptors in the CNS to suppress pain. In some embodiments, the peptide is capable of reducing a level of pain in a subject upon administration.

Kinds of pain include chronic pain, acute pain, nociceptive pain, neuropathic pain, allodynia, phantom pain, visceral pain, breakthrough pain. Neurotensin is a non-opioid but can provide analgesia. Opioid can cause addiction and neurotensin delivery to the CNS offers the possibility of non-addictive pain relief. Neurotensin peptide fusions can also be used to treat dopaminergic, serotonergic, GABAergic, glutamatergic, and cholinergic systems, schizophrenia, psychosis, drug abuse, Parkinson's disease (PD), pain, central control of blood pressure, eating disorders, as well as cancer and inflammation. Neurotensin peptide fusions can be used to treat pain including chronic pain, acute pain, injury, mechanical pain, heat pain, cold pain, ischemic pain, and chemical-induced pain, inflammatory pain, migraine-related pain, headache-related pain, irritable bowel syndrome-related pain, fibromyalgia-related pain, arthritic pain, skeletal pain, joint pain, gastrointestinal pain, muscle pain, angina pain, facial pain, pelvic pain, claudication, postoperative pain, post traumatic pain, tension-type headache, obstetric pain, gynecological pain, or chemotherapy-induced pain, nociceptive pain, neuropathic pain, allodynia, phantom pain, visceral pain, breakthrough pain, familial episodic pain syndrome, paroxysmal extreme pain disorder, congenital indifference to pain, pain associated with opioid withdrawal. Neurotensin peptide fusions can be used in neuroendocrine regulation, such as of the ACTH axis, the HPA axis, hormone regulation, and ion channel regulation, such as in diseases and disorders including Bartter's syndrome, Andersen's syndrome, congenital hyperinsulinism, neonatal diabetes, dilated cardiomyopathy, episodic ataxia type 1, long QT syndrome, short QT syndrome, benign neonatal febrile convulsions, nonsyndromic deafness, long QT syndrome, short QT syndrome, polycystic kidney disease, familial episodic pain syndrome, focal segmental glomerulosclerosis, retinitis pigmentosa, epilepsy, severe myoclonic epilepsy, long QT syndrome, cerebellar ataxia, erythromelalgia, paroxysmal extreme pain disorder, congenital indifference to pain, benign familial neonatal seizures, timothy syndrome, episodic ataxia type 2, stiff baby syndrome, juvenile myoclonic epilepsy, and autosomal dominant nocturnal frontal lobe epilepsy. In some embodiments, peptide oligonucleotide complexes (e.g., peptide-NT containing oligonucleotide complexes) interact with dopamine-signaling neurons to reduce pain.

In some embodiments, the TfR-binding peptides of the present disclosure can be used for the treatment and prevention of various neurological diseases including but not limited to epilepsy, schizophrenia, depression, anxiety, bipolar disorder, developmental brain disorders (e.g., autism spectrum), or mood disorder.

Binding of the herein described engineered peptide constructs and peptide complexes (e.g., peptide oligonucleotide complexes, peptide conjugates, fusion peptides, or recombinantly produced peptide constructs) to TfR and subsequent transport across a cell layer or barrier such as the BBB (e.g., via vesicular transcytosis) or a cell membrane (e.g., via endocytosis) can have implications in a number of diseases, conditions, or disorders associated with neurodegeneration. Neurodegenerative diseases that can treated, prevented, or diagnosed with the herein described TfR-binding peptides can include Alzheimer's disease, Amyotrophic lateral sclerosis, Friedreich's ataxia, Huntington's disease, Lewy body disease, Parkinson's disease, Spinal muscular atrophy, Motor neuron disease, Lyme disease, Ataxia-telangiectasia, Autosomal dominant cerebellar ataxia, Batten disease, Corticobasal syndrome, Creutzfeldt-Jakob disease, Fragile X-associated tremor/ataxia syndrome, Kufor-Rakeb syndrome, Machado-Joseph disease, multiple sclerosis, chronic traumatic encephalopathy, or frontotemporal dementia. In some embodiments, the TfR-binding peptide can be used in combination with BACE inhibitors, galantamine, amantadine, benztropine, biperiden, bromocriptin, carbidopa, donepezil, entacapone, levodopa, pergolie, pramipexole, procyclidine, rivastigmine, ropinirole, selegiline, tacrine, tolcapone, or trihexyphenidyl to treat and/or prevent a neurodegenerative disease.

In some embodiments, modulation (e.g., inhibition) of ion channels such as Kᵥ1.3 potassium channels using TfR-binding peptides and ion channel modulator such as Kᵥ1.3 potassium channel inhibitor conjugates oligonucleotide complexes can be used for treating or preventing inflammation in the brain. Kᵥ1.3 potassium channels, for example, can be highly expressed on microglia in the brain. Thus, neuroinflammatory and neurodegenerative diseases such as multiple sclerosis, Alzheimer's, Parkinson's, traumatic brain injury, radiation therapy toxicity and other neurodegenerative and neuroinflammatory diseases can be treated with TfR-binding peptides that are conjugated to, linked to, or fused to a Kᵥ1.3 inhibitor. These diseases can be marked by upregulation of Kᵥ1.3. In some cases, Kᵥ1.3 inhibition can be used for treatment of psoriasis and other non-brain autoimmune diseases due to its effect on effector T cells. In some embodiments, a Kᵥ1.3 inhibitor peptide may be Vm24 (SEQ ID NO: 359 or SEQ ID NO: 362, GSAAAISCVGSPECPPKCRAQGCKNGKCMNRKCKCYYC) or ShK-186 (SEQ ID NO: 360). A Kᵥ1.3 inhibitor peptide may be conjugated to any of the peptides disclosed herein (e.g., any one of SEQ ID NO: 1-SEQ ID NO: 134 or SEQ ID NO: 306-SEQ ID NO: 335). Exemplary Kᵥ1.3 inhibitor peptides are provided in TABLE 9.

TABLE 9
Exemplary Kv1.3 Inhibitor Peptides

| SEQ ID NO | Amino Acid Sequence |
|---|---|
| SEQ ID NO: 359 | AAAISCVGSPECPPKCRAQGCKNGKCMNRKCKCYYC |
| SEQ ID NO: 360 | RSCIDTIPKSRCTAFQCKHSMKYRLSFCRKTCGTC |
| SEQ ID NO: 361 | GSEWSSRSCIDTIPKSRCTAFQCKHSMKYRLSFCRKTCGTC |
| SEQ ID NO: 362 | GSAAAISCVGSPECPPKCRAQGCKNGKCMNRKCKCYYC |
| SEQ ID NO: 363 | EWSSRSCIDTIPKSRCTAFQCKHSMKYRLSFCRKTCGTC |

In some embodiments, a Kᵥ1.3 inhibitor, for example a Kᵥ1.3 peptide inhibitor, may be conjugated to the TfR-binding peptides of the present disclosure. In some embodiments, a Kᵥ1.3 peptide inhibitor may comprise a linker for fusing or conjugating to a TfR-binding peptide. In some embodiments, a Kᵥ1.3 peptide inhibitor fused to a TfR-binding peptide may comprise a peptide with a sequence of SEQ ID NO: 361 or SEQ ID NO: 363. In some embodiments, a linker may have a sequence of any one of SEQ ID NO: 234-SEQ ID NO: 297. A Kᵥ1.3 binding peptide (e.g., SEQ ID NO: 359, SEQ ID NO: 360, SEQ ID NO: 362, or SEQ ID NO: 363) may be conjugated to a TfR-binding peptide with a sequence of any one of SEQ ID NO: 1-SEQ ID NO: 134 or SEQ ID NO: 306-SEQ ID NO: 335.

In some embodiments, the TfR-binding peptides of the present disclosure can be used for the treatment and prevention of Crohn's disease or, more generally, inflammatory bowel diseases. In some cases, the TfR-binding peptides of the present disclosure show high uptake and retention in glandular cells of the intestine, which can express high amounts of TfR.

In some embodiments, the TfR-binding peptides as described herein can be used to treat and/or prevent cancers located both in and outside the CNS, including but not limited to ovarian cancer, colon cancer, glioblastoma, astrocytoma, glioma, medulloblastoma, ependymoma, choroid plexus carcinoma, midline glioma, diffuse intrinsic pontine glioma, and lung cancer, cancer located in the bone or bone marrow, glioblastoma, astrocytoma, glioma, medulloblastoma, ependymoma, choroid plexus carcinoma, midline glioma, and diffuse intrinsic pontine glioma (DIPG), breast cancer, liver cancer, colon cancer, brain cancer, spleen cancer, cancers of the salivary gland, kidney cancer, muscle cancers, bone marrow cell cancers, or skin cancer, genitourinary cancer, osteosarcoma, muscle-derived sarcoma, melanoma, head and neck cancer, a neuroblastoma, or a CMYC-overexpressing cancer.

In some embodiments, TfR-binding peptide constructs that can be used to prevent and/or treat a cancer are those comprising a TfR binding peptide and an active agent with anti-tumor activity such as an IL15, a fused IL15/ILT5Ra, IFNgamma, and anti-CD3 agents. These exemplary peptide constructs can comprise secretion murine IgKappa leader sequences, an anti-CD3 scFv, lysine to arginine mutants of the scFv, shorty-Flag sequences, His-tags, IL-15Ra, IL-15, IFN gamma, any linker disclosed herein, any peptides disclosed herein, and any fragments or derivatives thereof.

The peptides of SEQ ID NO: 1-SEQ ID NO: 134 or SEQ ID NO: 306-SEQ ID NO: 335 could further be fused to neurotensin. Each of said peptides or peptide-NT fusions may include a peptide of SEQ ID NO: 65, SEQ ID NO: 66, or SEQ ID NO: 96 (or SEQ ID NO: 1, SEQ ID NO: 2, or SEQ ID NO: 32 with no N-terminal GS), and each of which can further include an expression tag or sequence to improve protein expression, secretion, and/or purification, such as the genetic fusion expression tag siderocalin. In some embodiments, the TfR-binding peptides of the present disclosure are conjugated to, linked to, or fused to anti-cancer drugs that can include an EGFRvIII inhibitor, Copiktra (duvelisib), Erleada (apalutamide), Libtayo (cemiplimab-rwlc), Lorbrena (lorlatinib), Lumoxiti (moxetumomab pasudotox-tdf), Lutathera (lutetium Lu 177 dotatate), Talzenna (talazoparib), Vizimpro (dacomitinib), Aliqopa (copanlisib), Alunbrig (brigatinib), Bavencio (avelumab), Besponsa (inotuzumab ozogamicin), Calquence (acalabrutinib), IDHIFA (enasidenib), Imfinzi (durvalumab), Kisqali (ribociclib), Kymriah (tisagenlecleucel), Nerlynx (neratinib), Rydapt (midostaurin), Vyxeos (daunorubicin and cytarabine), Xermelo (telotristat ethyl), Yescarta (axicabtagene ciloleucel), Zejula (niraparib), Cabometyx (cabozantinib), Keytruda (pembrolizumab), Lartruvo (olaratumab), Lenvima (lenvatinib), Opdivo (nivolumab), Rubraca (rucaparib), Sustol (granisetron), Syndros (dronabinol oral solution), Tecentriq (atezolizumab), Venclexta (venetoclax), Alecensa (alectinib), Cotellic (cobimetinib), Darzalex (daratumumab), Empliciti (elotuzumab), Farydak (panobinostat), Ibrance (palbociclib), Imlygic (talimogene laherparepvec), Lenvima (lenvatinib), Lonsurf (trifluridine and tipiracil), Ninlaro (ixazomib), Odomzo (sonidegib), Onivyde (irinotecan liposome injection), Portrazza (necitumumab), Tagrisso (osimertinib), Unituxin (dinutuximab), Varubi (rolapitant), Vistogard (uridine triacetate), Yondelis (trabectedin), Akynzeo (netupitant and palonosetron), Beleodaq (belinostat), Blincyto (blinatumomab), Cyramza (ramucirumab), Imbruvica (ibrutinib), Lynparza (olaparib), Zydelig (idelalisib), Zykadia (ceritinib), Gazyva (obinutuzumab), Gilotrif (afatinib), Imbruvica (ibrutinib), Kadcyla (ado-trastuzumab emtansine), Mekinist (trametinib), Pomalyst (pomalidomide), Revlimid (lenalidomide), Stivarga (regorafenib), Tafinlar (dabrafenib), Valchlor (mechlorethamine) gel, Xgeva (denosumab), Xofigo (radium Ra 223 dichloride), Abraxane (paclitaxel protein-bound particles for injectable suspension), Afinitor (everolimus), Afinitor (everolimus), Bosulif (bosutinib), Cometriq (cabozantinib), Erivedge (vismodegib), Iclusig (ponatinib), Inlyta (axitinib), Kyprolis (carfilzomib), Marqibo (vinCRIStine sulfate LIPOSOME injection), Neutroval (tbo-filgrastim), Perjeta (pertuzumab), Picato (ingenol mebutate) gel, Stivarga (regorafenib), Subsys (fentanyl sublingual spray), Synribo (omacetaxine mepesuccinate), Votrient (pazopanib), Xtandi (enzalutamide), Zaltrap (ziv-aflibercept), Abstral (fentanyl sublingual tablets), Adcetris (brentuximab vedotin), Afinitor (everolimus), Erwinaze (asparaginase Erwinia chrysanthemi), Lazanda (fentanyl citrate) nasal spray, Sutent (sunitinib malate), Sylatron (peginterferon alfa-2b), Vandetanib (vandetanib), Xalkori (crizotinib), Yervoy (ipilimumab), Zelboraf (vemurafenib), Zytiga (abiraterone acetate), Halaven (eribulin mesylate), Herceptin (trastuzumab), Jevtana (cabazitaxel), Provenge (sipuleucel-T), Xgeva (denosumab), Zuplenz (ondansetron oral soluble film), Afinitor (everolimus), Arzerra (ofatumumab), Avastin (bevacizumab), Cervarix [Human Papillomavirus Bivalent (Types 16 and 18) Vaccine, Recombinant, Elitek (rasburicase), Folotyn (pralatrexate injection), Istodax (romidepsin), Onsolis (fentanyl buccal), Votrient (pazopanib), Degarelix (degarelix for injection), Fusilev (levoleucovorin); Mozobil (plerixafor injection), Sancuso (granisetron), Treanda (bendamustine hydrochloride), Evista (raloxifene hydrochloride), Hycamtin (topotecan hydrochloride), Ixempra (ixabepilone), Tasigna (nilotinib hydrochloride monohydrate), Torisel (temsirolimus), Tykerb (lapatinib), Gardasil (quadrivalent human papillomavirus (types 6, 11, 16, 18) recombinant vaccine), Sprycel (dasatinib), Sutent (sunitinib), Vectibix (panitumumab), Arranon (nelarabine), Nexavar (sorafenib), Alimta (pemetrexed for injection), Avastin (bevacizumab); Clolar (clofarabine), Erbitux (cetuximab), Sensipar (cinacalcet), Tarceva (erlotinib, OSI 774), Aloxi (palonosetron), Emend (aprepitant), Iressa (gefitinib); Plenaxis (abarelix for injectable suspension); Premarin (conjugated estrogens); UroXatral (alfuzosin HCl extended-release tablets); Velcade (bortezomib); Eligard (leuprolide acetate); Eloxatin (oxaliplatin/5-fluorouracil/leucovorin); Faslodex (fulvestrant); Gleevec (imatinib mesylate); Neulasta; SecreFlo (secretin); Zevalin (ibritumomab tiuxetan); Zometa (zoledronic acid); Campath; Femara (letrozole); Gleevec (imatinib mesylate); Kytril (granisetron) solution; Trelstar LA (triptorelin pamoate); Xeloda; Zometa (zoledronic acid); Mylotarg (gemtuzumab ozogamicin); Trelstar Depot (triptorelin pamoate); Trisenox (arsenic trioxide); Viadur (leuprolide acetate implant); Aromasin Tablets; Busulflex; Doxil (doxorubicin HCl liposome injection); Ellence; Ethyol (amifostine); Temodar; UVADEX Sterile Solution; Zofran; Actiq; Anzemet; Camptosar; Gemzar (gemcitabine HCL); Herceptin; Inform HER-2/neu breast cancer test; Neupogen; Nolvadex; Photofrin; Proleukin; Sclerosol Intrapleural Aerosol; Valstar; Xeloda; Zofran; Anzemet; Bromfenac; Femara (letrozole); Gliadel Wafer (polifeprosan 20 with carmustine implant); Intron A (interferon alfa-2b, recombinant); Kytril (granisetron) tablets; Lupron Depot (leuprolide acetate for depot suspension); Miraluma test; Neumega; Quadramet (Samarium Sm 153 Lexidronam Injection); Rituxan; Taxol; Anexsia; Aredia (pamidronate disodium for injection); Arimidex (anastrozole); Campostar; Elliotts B Solution (buffered intrathecal electrolyte/dextrose injection); Eulexin (flutamide); Gemzar (gemcitabine HCL); Hycamtin (topotecan hydrochloride); Kadian; Leukine (sargramostim); Lupron Depot (leuprolide acetate for depot suspension); Photodynamic Therapy; Taxotere (Docetaxel); Visipaque (iodixanol); Ethyol (amifostine); Intron A (Interferon alfa-2b, recombinant), and Leukine (sargramostim). In some embodiments, the TfR-binding peptides of the present disclosure are conjugated to, linked to, or fused to Abemaciclib, Acalabrutinib, Afatinib, Alectinib, Axitinib, Baricitinib, Binimetinib, Bosutinib, Brigatinib, Cabozantinib, Ceritinib, Cobimetinib, Crizotinib, Dabrafenib, dacomitinib, Dasatinib, encorafenib, Erlotinib, Everolimus, Fostamatinib, Gefitinib, Ibrutinib, Imatinib, Lapatinib, Lenvatinib, Lorlatinib, Midostaurin, Neratinib, Nilotinib, Nintedanib, Osimertinib, Palbociclib, Pazopanib, Ponatinib, Regorafenib, Ribociclib, Ruxolitinib, Sirolimus, Sorafenib, Sunitinib, Temsirolimus, Tofacitinib, Trametinib, Vandetanib, Vemurafenib, an IL15, a fused IL15/IL15Ra, an IFNgamma, and anti-CD3 agents, or fragment(s) thereof.

In some embodiments, the TfR-binding peptides as described herein can be used to treat and/or prevent an infection including infections of the central nervous system. In some embodiments, the TfR-binding peptides of the present disclosure are conjugated to, linked to, or fused to an anti-infective agent. Anti-infective agents can include antibiotic, antiviral, and antifungal agents. Anti-infective agent that can be used include artemisinin, Aemcolo (rifamycin), Biktarvy (bictegravir/emtricitabine/tenofovir alafenamide), Nuzyra (omadacycline), Trogarzo (ibalizumab-uiyk), Xofluza (baloxavir marboxil), Baxdela (delafloxacin) tablets and injection, Benznidazole, Giapreza (angiotensin II), Heplisav-B [Hepatitis B Vaccine (Recombinant), Adjuvanted], Juluca (dolutegravir and rilpivirine), KedRab [Rabies Immune Globulin (Human)], Mavyret (glecaprevir and pibrentasvir), Prevymis (letermovir), Shingrix (Zoster Vaccine Recombinant, Adjuvanted), Solosec (seenidazole), Vabomere (meropenem and vaborbactam), Xepi (ozenoxacin), Anthim (obiltoxaximab), Descovy (emtricitabine and tenofovir alafenamide), Epclusa (sofosbuvir and velpatasvir), Odefsey (emtricitabine, rilpivirine, and tenofovir alafenamide), Vaxchora (Cholera Vaccine, Live, Oral), Vemlidy (tenofovir alafenamide), Zepatier (elbasvir and grazoprevir), Zinplava (bezlotoxumab), Avycaz (ceftazidime-avibactam), Bexsero (Meningococcal Group B Vaccine), Cresemba (isavuconazonium sulfate), Daklinza (daclatasvir), Evotaz (atazanavir and cobicistat), Fluad (trivalent influenza vaccine), Genvoya (elvitegravir, cobicistat, emtricitabine, and tenofovir alafenamide), Prezcobix (darunavir and cobicistat), Technivie, (ombitasvir, paritaprevir and ritonavir), Dalvance (dalbavancin), Harvoni (ledipasvir and sofosbuvir), Impavido (miltefosine), Kerydin (tavaborole), Orbactiv (oritavancin), Rapivab (peramivir injection), Sivextro (tedizolid phosphate), Triumeq (abacavir, dolutegravir, and lamivudine), Zerbaxa (ceftolozane+tazobactam), Flublok (seasonal influenza vaccine), Luzu (luliconazole) Cream 1%, Olysio (simeprevir), Sitavig (acyclovir) buccal tablets, Sovaldi (sofosbuvir), VariZIG, Varicella Zoster Immune Globulin (Human), Abthrax (raxibacumab), Afinitor (everolimus), Cystaran (cysteamine hydrochloride), Dymista (azelastine hydrochloride and fluticasone propionate), Flucelvax, Influenza Virus Vaccine, Jetrea (ocriplasmin), Linzess (linaclotide), Mytesi (crofelemer), Sirturo (bedaquiline), Sklice (ivermectin) lotion, Stribild (elvitegravir, cobicistat, emtricitabine, tenofovir disoproxil fumarate), Tudorza Pressair (aclidinium bromide inhalation powder), Afinitor (everolimus), Complera (emtricitabine/rilpivirine/tenofovir disoproxil fumarate), Dificid (fidaxomicin), Edurant (rilpivirine), Eylea (aflibercept), Firazyr (icatibant), Gralise (gabapentin), Incivek (telaprevir), Victrelis (boceprevir), Egrifta (tesamorelin for injection), Menveo (meningitis vaccine), Zymaxid (gatifloxacin ophthalmic solution), Afinitor (everolimus), Bepreve (bepotastine besilate ophthalmic solution), Hiberix (Haemophilus b Conjugate Vaccine, Tetanus Toxoid Conjugate), Vibativ (telavancin), Aptivus (tipranavir), Astepro (azelastine hydrochloride nasal spray), Intelence (etravirine), Patanase (olopatadine hydrochloride), Viread (tenofovir disoproxil fumarate), Isentress (raltegravir), Selzentry (maraviroc), Veramyst (fluticasone furoate), Xyzal (levocetirizine dihydrochloride), Eraxis (anidulafungin), Noxafil (posaconazole), Prezista (darunavir), Tyzeka (telbivudine), Veregen (kunecatechins), Aptivus (tipranavir), Baraclude (entecavir), FluMist (Influenza Virus Vaccine), Fuzeon (enfuvirtide), Lexiva (fosamprenavir calcium), Reyataz (atazanavir sulfate), Botox Cosmetic (botulinum toxin type A), Clarinex, Hepsera (adefovir dipivoxil), Pediarix Vaccine, Pegasys (peginterferon alfa-2a), Sustiva, Vfend (voriconazole), Peg-Intron (peginterferon alfa-2b), Rebetol (ribavirin), Spectracef, Tavist (clemastine fumarate), Twinrix, Valcyte (valganciclovir HCl), Viread (tenofovir disoproxil fumarate), Xigris (drotrecogin alfa [activated]), ABREVA (docosanol), Cefazolin and Dextrose USP, Children's Motrin Cold, REBETRON (TM) Combination Therapy, Synagis, Viroptic, Aldara (imiquimod), Ceftin (cefuroxime axetil), Combivir, Famvir (famciclovir), Floxin otic, Fortovase, INFERGEN (interferon alfacon-1), Intron A (interferon alfa-2b, recombinant), Taxol, Timentin, Trovan, VIRACEPT (nelfinavir mesylate), Zerit (stavudine), AK-Con-A (naphazoline ophthalmic), Allegra (fexofenadine hydrochloride), Atrovent (ipratropium bromide), Augmentin (amoxicillin/clavulanate), Crixivan (Indinavir sulfate), Elmiron (pentosan polysulfate sodium), Havrix, IvyBlock, Leukine (sargramostim), Merrem I.V. (meropenem), Tavist (clemastine fumarate), Videx (didanosine), Viramune (nevirapine), Zithromax (azithromycin), Cedax (ceftibuten), Clarithromycin (Biaxin), Epivir (lamivudine), Invirase (saquinavir), Valtrex (valacyclovir HCl), Zerit (stavudine), Zyrtec (cetirizine HCl), or a functional fragment thereof.

In some embodiments, the TfR-binding peptides as described herein can be used to treat and/or prevent an inflammation including inflammation of the central nervous system and the gastrointestinal (GI) tract. In some embodiments, the TfR-binding peptides of the present disclosure are conjugated to, linked to, or fused to an anti-inflammatory agent. Anti-inflammatory agents can include steroidal and nonsteroidal anti-inflammatory agents. Nonsteroidal anti-inflammatory agents can include aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, piroxicam, flurbiprofen, and fenoprofen, Anti-inflammatory drugs that can be used in combination with the herein disclosed methods and compositions can include corticosteroids and aminosalicylates including but not limited to mesalamine, balsalazide, and olsalazine, In various cases, anti-inflammatory drugs include tumor necrosis factor (TNF)-alpha inhibitors such as infliximab, adalimumab, and golimumab, and biological drug (e.g., antibodies, fragments or derivatives thereof) targeting other biological targets including natalizumab, vedolizumab, and ustekinumab.

As compared to antibody-drug conjugates (e.g., Adcetris, Kadcyla, Mylotarg) or transferrin, in some aspects the peptide conjugated to, linked to, or fused to an active agent as described herein can exhibit better penetration of solid tumors or of the CNS due to its smaller size. In certain aspects, the peptide conjugated to, linked to, or fused to an active agent as described herein can carry different or higher doses of active agents as compared to antibody-drug conjugates. In some embodiments, the peptide as described conjugated to, linked to, or fused to an active agent as described herein can deliver higher doses of an active agent to skeletal muscle tissue compared to antibody-drug conjugates. Thus, treatment of muscular dystrophy, SBMA, or muscle-derived sarcomas can be of particular interest using the peptides of the present disclosure. In some aspects, the TfR-binding peptides as described herein can exhibit significantly lower plasma half-lives compared to antibodies or antibody-drug conjugates, and thus can allow for a more rapid peripheral clearance (can be advantageous e.g., when radiolabeled conjugates are used providing significantly lower organ dosimetries). In still other aspects, the peptide conjugated to, linked to, or fused to an active agent as described herein can have better site-specific delivery of defined drug ratio as compared to antibody-drug conjugates. In other aspects, the peptide can be amenable to solvation in organic solvents (in addition to water), which can allow more synthetic routes for solvation and conjugation of a drug (which often has low aqueous solubility) and higher conjugation yields, higher ratios of drug conjugated to, linked to, or fused to peptide (versus an antibody), and/or reduce aggregate/high molecular weight species formation during conjugation. Additionally, a unique amino acid residue(s) can be introduced into the peptide via a residue that is not otherwise present in the short sequence or via inclusion of a non-natural amino acid, allowing site specific conjugation to the peptide.

The peptide oligonucleotide complexes of the present disclosure can also be conjugated to, linked to, or fused to other moieties that can serve other roles, such as providing an affinity handle (e.g., biotin) for retrieval of the peptides from tissues or fluids. For example, peptide oligonucleotide complexes of the present disclosure can also be conjugated to, linked to, or fused to biotin. In addition to extension of half-life, biotin can also act as an affinity handle for retrieval of peptide oligonucleotide complexes from tissues or other locations. In some embodiments, fluorescent biotin conjugates that can act both as a detectable label and an affinity handle can be used. Non limiting examples of commercially available fluorescent biotin conjugates can include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexa fluor 488 biocytin, Alexa flour 546, Alexa Fluor 549, lucifer yellow cadaverine biotin-X, Lucifer yellow biocytin, Oregon green 488 biocytin, biotin-rhodamine and tetramethylrhodamine biocytin. In some other examples, the conjugates can include chemiluminescent compounds, colloidal metals, luminescent compounds, enzymes, radioisotopes, and paramagnetic labels. In some embodiments, the peptide described herein can also be attached to another molecule. For example, the peptide sequence also can be attached to another active agent (e.g., small molecule, peptide, polypeptide, polynucleotide, antibody, aptamer, cytokine, growth factor, neurotransmitter, an active fragment or modification of any of the preceding agents, fluorophore, radioisotope, radionuclide chelator, acyl adduct, chemical linker, or sugar). In some embodiments, the peptide can be conjugated to, linked to, or fused with, or covalently or non-covalently linked to an active agent.

In some embodiments, peptide oligonucleotide complexes of the present disclosure can also be conjugated to, linked to, or fused to other affinity handles. Other affinity handles may include genetic fusion affinity handles. Genetic fusion affinity handles may include 6×His (HHHHHH (SEQ ID NO: 336); immobilized metal affinity column purification possible), FLAG (DYKDDDDK (SEQ ID NO: 337); anti-FLAG immunoprecipitation), “shorty” FLAG (DYKDE (SEQ ID NO: 338); anti-FLAG immunoprecipitation), hemagglutinin (YPYDVPDYA (SEQ ID NO: 339); anti-HA immunoprecipitation), and streptavidin binding peptide (DVEAWLGAR (SEQ ID NO: 340); streptavidin-mediated precipitation). In some embodiments, peptide oligonucleotide complexes of the present disclosure can also be conjugated to, linked to, or fused to an expression tag or sequence to improve protein expression and/or purification. Such expression tags may include genetic fusion expression tags. Genetic fusion expression tags may include siderocalin (SEQ ID NO: 342, METDTLLLWVLLLWVPGSTGDYKDEHHHHHHGGSQDSTSDLIPAPPLSKVPLQQNFQD NQFQGKWYVVGLAGNAILREDKDPQKMYATIYELKEDKSYNVTSVLFRKKKCDYWIR TFVPGSQPGEFTLGNIKSYPGLTSYLVRVVSTNYNQHAMVFFKKVSQNREYFKITLYGR TKELTSELKENFIRFSKSLGLPENHIVFPVPIDQCIDGGGSENLYFQ). Exemplary siderocalin peptide fusions include human soluble transferrin fused to siderocalin (METDTLLLWVLLLWVPGSTGDYKDEHHHHHHGGSQDSTSDLIPAPPLSKVPLQQNFQ DNQFQGKWYVVGLAGNAILREDKDPQKMYATIYELKEDKSYNVTSVLFRKKKCDYWI RTFVPGSQPGEFTLGNIKSYPGLTSYLVRVVSTNYNQHAMVFFKKVSQNREYFKITLYG RTKELTSELKENFIRFSKSLGLPENHIVFPVPIDQCIDGGGSENLYFQGSRLYWDDLKRKL SEKLDSTDFTSTIKLLNENSYVPREAGSQKDENLALYVENQFREFKLSKVWRDQHFVKI QVKDSAQNSVIIVDKNGRLVYLVENPGGYVAYSKAATVTGKLVHANFGTKKDFEDLY TPVNGSIVIVRAGKITFAEKVANAESLNAIGVLIYMDQTKFPIVNAELSFFGHAHLGTGD PYTPGFPSFNHTQFPPSRSSGLPNIPVQTISRAAAEKLFGNMEGDCPSDWKTDSTCRMVT SESKNVKLTVSNVLKEIKILNIFGVIKGFVEPDHYVVVGAQRDAWGPGAAKSGVGTALL LKLAQMFSDMVLKDGFQPSRSIIFASWSAGDFGSVGATEWLEGYLSSLHLKAFTYINLD KAVLGTSNFKVSASPLLYTLIEKTMQNVKHPVTGQFLYQDSNWASKVEKLTLDNAAFP FLAYSGIPAVSFCFCEDTDYPYLGTTMDTYKELIERIPELNKVARAAAEVAGQFVIKLTH DVELNLDYERYNSQLLSFVRDLNQYRADIKEMGLSLQWLYSARGDFFRATSRLTTDFG NAEKTDRFVMKKLNDRVMRVEYHFLSPYVSPKESPFRHVFWGSGSHTLPALLENLKLR KQNNGAFNETLFRNQLALATWTIQGAANALSGDVWDIDNEFGGGSHHHHHHGGGSLN DIFEAQKIEWHE; SEQ ID NO: 343) and various peptides of the present disclosure fused to siderocalin, such as METDTLLLWVLLLWVPGSTGDYKDEHHHHHHGGSQDSTSDLIPAPPLSKVPLQQNFQD NQFQGKWYVVGLAGNAILREDKDPQKMYATIYELKEDKSYNVTSVLFRKKKCDYWIR TFVPGSQPGEFTLGNIKSYPGLTSYLVRVVSTNYNQHAMVFFKKVSQNREYFKITLYGR TKELTSELKENFIRFSKSLGLPENHIVFPVPIDQCIDGGGSENLYFQGSREGCASRCTKYN AELEKCEARVSSMSNTEETCVQELFDLLHCVDHCVSQ) (SEQ ID NO: 346), METDTLLLWVLLLWVPGSTGDYKDEHHHHHHGGSQDSTSDLIPAPPLSKVPLQQNFQD NQFQGKWYVVGLAGNAILREDKDPQKMYATIYELKEDKSYNVTSVLFRKKKCDYWIR TFVPGSQPGEFTLGNIKSYPGLTSYLVRVVSTNYNQHAMVFFKKVSQNREYFKITLYGR TKELTSELKENFIRFSKSLGLPENHIVFPVPIDQCIDGGGSENLYFQGSREGCASRCTKYN AELEKCEARVMSMSNTEEDCEQELEDLLHCLDHCHSQ (SEQ ID NO: 347), and (METDTLLLWVLLLWVPGSTGDYKDEHHHHHHGGSQDSTSDLIPAPPLSKVPLQQNFQ DNQFQGKWYVVGLAGNAILREDKDPQKMYATIYELKEDKSYNVTSVLFRKKKCDYWI RTFVPGSQPGEFTLGNIKSYPGLTSYLVRVVSTNYNQHAMVFFKKVSQNREYFKITLYG RTKELTSELKENFIRFSKSLGLPENHIVFPVPIDQCIDGGGSENLYFQGSREGCASRCMKY NDELEKCEARMMSMSNTEEDCEQELEDLLYCLDHCHSQ (SEQ ID NO: 348). Notably CDP-NT peptide constructs can be expressed with N-terminal siderocalin.

In some embodiments, peptide oligonucleotide complexes of the present disclosure can also be conjugated to, linked to, or fused to other moieties that can serve other roles, such as providing an affinity handle (e.g., biotin) for recruiting a non-conjugated therapeutic to or into the tissue that is targeted such as a tumor. In some cases, the therapeutic is a small molecule, peptide, antibody, or cellular therapeutic (e.g., a CAR T cell or another autologous or allogeneic engineered cell). For example, CAR T cells engineered with an affinity label (e.g., avidin) can be recruited to tissues that are targeted by the TfR-binding peptides that comprise a biotin moiety. In addition, biotin- (or other affinity labeled)-TfR-binding peptides as described herein can be administered (e.g., co-administered) with a variety of different avidin-tagged drugs, such as small molecules, peptides, or proteins that can provide pharmacologic activity in the brain, a tumor, or other tissues that the TfR-binding peptides target or accumulate in.

Additionally, more than one peptide sequence derived from a toxin or knotted venom protein can be present on, conjugated to, linked to, or fused with a particular peptide. A peptide can be incorporated into a biomolecule by various techniques. A peptide can be incorporated by a chemical transformation, such as the formation of a covalent bond, such as an amide bond. A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule or can substitute for a subsequence of the sequence that encodes the biomolecule.

### Detectable Agent Peptide Conjugates

A peptide oligonucleotide complex of the present disclosure (e.g., a peptide oligonucleotide complex comprising a TfR-binding peptide and a nucleotide) may be further conjugated, linked, or fused to a detectable agent. In some embodiments, the detectable agent may be directly or indirectly linked to the peptide of the peptide oligonucleotide complex or the nucleotide of the peptide oligonucleotide complex. A peptide nucleic acid complex further comprising a detectable agent may be referred to as a detectable agent peptide conjugate. A peptide nucleotide complex can be conjugated to, linked to, or fused to an agent used in imaging, research, therapeutics, theranostics, pharmaceuticals, chemotherapy, chelation therapy, targeted drug delivery, and radiotherapy. In some embodiments, a peptide is conjugated to, linked to, or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging.

In some embodiments, 1, 2, 3, 4, 5, 6, 7, 8, 9, or 10 detectable agents can be linked to a peptide or nucleotide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177.

In some embodiments, A peptide oligonucleotide complex of the present disclosure (e.g., a peptide oligonucleotide complex comprising a TfR-binding peptide and a nucleotide) further comprise a radioisotope, radiochelator, radiosensitizer, or photosensitizer. In some embodiments, the radioisotope, radiochelator, radiosensitizer, or photosensitizer may be incorporated into, or directly or indirectly linked to the peptide of the peptide oligonucleotide complex or the nucleotide of the peptide oligonucleotide complex. The radioisotope, radiochelator, radiosensitizer, photosensitizer may function as a detectable or therapeutic moiety. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. Additionally, the following radionuclides can be used for diagnosis and/or therapy: carbon (e.g., ¹¹C or ¹⁴C), nitrogen (e.g., ¹³N), fluorine (e.g., ¹⁸F), gallium (e.g., ⁶⁷Ga or ⁶⁸Ga), copper (e.g., ⁶⁴Cu or ⁶⁷Cu), zirconium (e.g., ⁸⁹Zr), lutetium (e.g., ¹⁷⁷Lu).

Peptides or nucleotides of a peptide oligonucleotide complex can be conjugated to, linked to, or fused to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers can include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach can allow for highly specific targeting of diseased cells (e.g., cancer cells) using both a therapeutic agent (e.g., drug) and electromagnetic energy (e.g., radiation or light) concurrently. In some embodiments, the peptide is conjugated to, linked to, fused with, or covalently or non-covalently linked to the agent, e.g., directly or via a linker. Exemplary linkers suitable for use with the embodiments herein are discussed in further detail below.

In some embodiments, a peptide oligonucleotide complex of the present disclosure (e.g., a peptide oligonucleotide complex comprising a TfR-binding peptide and a nucleotide) may be conjugated, linked, or fused to a radionuclide via chelator. In some embodiments, the radionuclide may be linked to the peptide of the peptide oligonucleotide complex or the nucleotide of the peptide oligonucleotide complex via the chelator. In some aspects of the present disclosure, the radionuclide is attached to a peptide oligonucleotide complex as described herein using a chelator. Exemplary chelator moieties can include 2,2′,2′-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid; 2,2′,2′-(3-(4-(3-(1-(4-(1,2,4,5-tetrazin-3-yl)phenyl)-1-oxo-5,8,11,14,17,20,23,26,29,32,35-undecaoxa-2-azaheptatriacontain-37-yl)thioureido)benzyl)-1,4,7-triazonane-2,5,8-triyl)triacetic acid; 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontain-37-yl)thioureido)benzyl)-1,4,7-triazonane-1,4-diyl)diacetic acid; 2,2′,2′-(3-(4-(3-(1-(4-(1,2,4,5-tetrazin-3-yl)phenyl)-3,7-dioxo-11,14,17,20,23,26,29-heptaoxa-2,8-diazahentriacontain-31-yl)thioureido)benzyl)-1,4,7-triazonane-2,5,8-triyl)triacetic acid; 2,2,2′-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontain-43-yl)thioureido)benzyl)-1,4,7-triazonane-2,5,8-triyl)triacetic acid; 2,2′,2′-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid; 2,2′,2′-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid; 2,2′,2′-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid; 2,2′,2′-(2-(4-(1-(4-(6-methyl-1,2,4,5-tetrazin-3-yl)phenoxy)-3,6,9,12,15,18,21,24,27,30,33-undecaoxahexatriacontain-36-amido)benzyl)-1,4,7-triazonane-1,4,7-triyl)triacetic acid; 2,2′,2′-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid; 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid; 2,2,2′-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and 2,2′,2′-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

### Linkers

A peptide oligonucleotide complex of the present disclosure (e.g., a peptide oligonucleotide complex comprising a TfR-binding peptide and a nucleotide) may be linked to an active agent or a detectable agent via a linker. In some embodiments, the linker may be conjugated to the peptide of the peptide oligonucleotide complex or the nucleotide of the peptide oligonucleotide complex. In some embodiments, a linker may connect the peptide of the peptide oligonucleotide complex to the nucleotide of the peptide oligonucleotide complex.

Peptides or nucleotides according to the present disclosure can be attached one another or to an additional cargo molecule (e.g., an additional therapeutically active agent or a detectable agent), such as one or more of a small molecule, another peptide, a protein, an antibody, an antibody fragment, an aptamer, polypeptide, polynucleotide, a fluorophore, a radioisotope, a radionuclide chelator, a polymer, a biopolymer, a fatty acid, an acyl adduct, a chemical linker, or sugar or other active agent or detectable agent described herein through a linker, or directly in the absence of a linker. In case a linker is absent, a cargo molecule is conjugated, linked, or fused directly to the N-terminus, the C-terminus, or an amino acid of a peptide. In cases where a linker is present, the linker can be a cleavable or stable linker. Cleavable linkers can be used for in vivo delivery of a cargo molecule, as a linker can be cleaved upon entry in a tissue, cell, endosome, or a nucleus. Stable linkers can be used for delivery of a cargo molecule that is active when conjugated or is degraded such as by catabolism. As described herein, a linker can also be used to covalently attach a TfR-binding peptide to another a cargo moiety, such as an oligonucleotide, having a separate function, such a targeting, cytotoxic, therapeutic, homing, imaging, or diagnostic functions.

In some embodiments, a molecule (e.g., a nucleotide or an additional active agent) can be conjugated to, linked to, or fused to the N-terminus and/or the C-terminus of a peptide to create an active agent or detectable agent peptide construct or a peptide oligonucleotide complex. In some embodiments, the TfR-targeting peptide can be attached at the N-terminus, an internal lysine, glutamic acid, or aspartic acid residue, or the C-terminus of another functional peptide or protein via a linker. An oligonucleotide can advantageously be conjugated to the N-terminus of a TfR-targeting peptide of this disclosure, as the TfR-targeting peptides of this disclosure have already been shown to be tolerant and TfR-binding when fused to another moiety at the N-terminus. In some embodiments, the peptide can be attached to another functional peptide or protein via a side chain, such as the side chain of a lysine, serine, threonine, cysteine, tyrosine, aspartic acid, a non-natural amino acid residue, or glutamic acid residue. As used herein, molecules can be linked, attached, or fused to each other via an amide bond, an ester bond, an ether bond, a carbamate bond, a carbonate bond, a carbon-nitrogen bond, a triazole, a macrocycle, an oxime bond, a thioester bond, a thioether bond a hydrazone bond, an azo bond, a carbon-carbon single, double, or triple bond, a disulfide bond, a two carbon bridge between two cysteines, a three carbon bridge between two cysteines, or a thioether bond. In some embodiments, the peptide comprises one or more non-natural amino acid, wherein the non-natural amino acid can be an insertion, appendage, or substitution for another amino acid. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single, double, or triple bond, a disulfide bond, a thioether bond, or other linker as described herein) can be used to link other molecules. In some embodiments, the peptide is attached to a terminus of the amino acid sequence of a larger polypeptide or peptide molecule, or is attached to a side chain, such as the side chain of a lysine, serine, threonine, cysteine, tyrosine, aspartic acid, a non-natural amino acid residue, or glutamic acid residue.

Attachment via a linker can involve incorporation of a linker moiety between a first molecule (e.g., an oligonucleotide, an additional active agent, and/or a detectable agent) and a second molecule (e.g., the peptide or the oligonucleotide). For example, a linker moiety may be incorporated between the nucleotide and the peptide of a peptide oligonucleotide complex, between the nucleotide and an additional active agent, or between the peptide and the additional active agent. The linker moiety may be any linker described herein (e.g., the linkers provided in TABLE 10 and TABLE 11). The first molecule and the second molecule can both be covalently attached to the linker. The linker can be cleavable, stable, self-immolating, hydrophilic, or hydrophobic. The linker can have bulky side groups or chains that sterically limit access of enzymes, water, or other chemicals to the linking group. The linker can have at least two functional groups, one bonded to the other molecule, and one bonded to the peptide, and a linking portion between the two functional groups. Some example linkers are described in Jain, N., Pharm Res. 32(11): 3526-40 (2015), Doronina, S. O., Bioconj Chem. 19(10): 1960-3 (2008), Pillow, T. H., J Med Chem. 57(19): 7890-9 (2014), Dorywalksa, M., Bioconj Chem. 26(4): 650-9 (2015), Kellogg, B. A., Bioconj Chem. 22(4): 717-27 (2011), and Zhao, R. Y., J Med Chem. 54(10): 3606-23 (2011).

Non-limiting examples of the functional groups for attachment can include functional groups capable of forming, for example, an amide bond, an ester bond, an ether bond, a carbonate bond, a carbamate bond, a carbon-nitrogen bond, a triazole, a macrocycle, an oxime bond, a hydrazone bond, a carbon-carbon single, double, or triple bond, a disulfide bond or a thioether bond. Non-limiting examples of functional groups capable of forming such bonds can include amino groups; carboxyl groups; hydroxyl groups; aldehyde groups; azide groups; alkyne and alkene groups; ketones; hydrazides; hydrazines; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; maleimides; linkers containing maleimide groups that are designed to hydrolyze; maleimidocaproyl; MCC ([N-maleimidomethyl]cyclohexane-1-carboxylate); N-ethylmaleimide; maleimide alkane; mc-vc-PABC; DUBA (DuocarmycinhydroxyBenzamide-Azaindole linker); SMCC Succinimidyl-4-(N-maleimidomethyl) cyclohexane-1-carboxylate; SPDP (N-succinimidyl-3-(2-pyridyldithio) propionate); SPDB N-succinimidyl-4-(2-pyridyldithio) butanoate; sulfo-SPDB N-succinimidyl-4-(2-pyridyldithio)-2-sulfo butanoate; SPP N-succinimidyl 4-(2-pyridyldithio)pentanoate; a dithiopyridylmaleimide (DTM); a hydroxylamine, a vinyl-halo group; haloacetamido groups; bromoacetamido; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion can include alkylene, alkenylene, alkynylene, polyether, such as polyethylene glycol (PEG), hydroxy carboxylic acids, polyester, polyamide, polyamino acids, polypeptides, cleavable peptides, Val-Cit, Phe-Lys, Val-Lys, Val-Ala, other peptide linkers as given in Doronina et al., 2008, linkers cleavable by beta glucuronidase, linkers cleavable by a cathepsin or by cathepsin B, D, E, H, L, S, C, K, O, F, V, X, or W, Val-Cit-p-aminobenzyloxycarbonyl, glucuronide-MABC, valine-citrulline, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, charged groups, zwitterionic groups, and ester groups. Other non-limiting examples of reactions to link, fuse, or conjugate molecules together include click chemistry (copper-accelerated azide-alkyne cycloaddition), copper-free click chemistry (strain-promoted azide-alkyne cycloaddition), HIPS ligation, Staudinger ligation, and hydrazine-iso-Pictet-Spengler.

In some embodiments, a linker can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acid residues in length. A linker can be about 10 amino acid residues in length. A linker can be about 1 amino acid residues in length. A linker can be about 2 amino acid residues in length. A linker can be about 3 amino acid residues in length. A linker can be about 4 amino acid residues in length. A linker can be about 5 amino acid residues in length. A linker can be about 11 amino acid residues in length. A linker can be about 12 amino acid residues in length. A linker can be about 13 amino acid residues in length. A linker can be about 14 amino acid residues in length. A linker can be about 15 amino acid residues in length. A linker can be about 16 amino acid residues in length. A linker can be about 17 amino acid residues in length. A linker can be about 18 amino acid residues in length. A linker can be about 19 amino acid residues in length. A linker can be about 20 amino acid residues in length. A linker can be about 21 amino acid residues in length. A linker can be about 22 amino acid residues in length. A linker can be about 23 amino acid residues in length. A linker can be about 24 amino acid residues in length. A linker can be about 25 amino acid residues in length. A linker can be about 26 amino acid residues in length. A linker can be about 27 amino acid residues in length. A linker can be about 28 amino acid residues in length. A linker can be about 29 amino acid residues in length. A linker can be about 30 amino acid residues in length.

In some embodiments, a linker of the present disclosure can comprise a cleavable or stable linker moiety. In some embodiments, cleavable linkers of the present disclosure can include, for example, protease cleavable peptide linkers, nuclease sensitive nucleic acid linkers, lipase sensitive lipid linkers, glycosidase sensitive carbohydrate linkers, pH sensitive linkers, hydrolytic linkers, reducible linkers, hypoxia sensitive linkers, photo-cleavable linkers, heat-labile linkers, enzyme cleavable linkers (e.g., esterase cleavable linker), ultrasound-sensitive linkers, and X-ray cleavable linkers. In some embodiments, the linker is not a cleavable linker.

A linker can comprise multiple amino acids. A linker can comprise 1, 2, 3, 4, 5, 6, 7, 8, 9, 10, 20 or more amino acids. A linker can comprise any of the linkers in the below TABLE 10 (where X=6-azidohexanoic acid and Z=citrulline). In some cases, an active agent including an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter can be attached to a peptide using any one or more of the linkers shown below in TABLE 10. In some embodiments, a linker provided in TABLE 10 can link a nucleotide to a peptide, an additional active agent, or a detectable agent. In some embodiments, a linker may comprise a sequence of any of SEQ ID NO: 234-SEQ ID NO: 297.

TABLE 10
Examples of Amino Acid Linkers

| SEQ ID NO | Sequence |
|---|---|
| SEQ ID NO: 234 | GGGS |
| SEQ ID NO: 235 | GₓS_{y} |
| SEQ ID NO: 236 | (GS)x |
| SEQ ID NO: 237 | GGSSG |
| SEQ ID NO: 238 | GGGGG |
| SEQ ID NO: 239 | GSGSGSGS |
| SEQ ID NO: 240 | GSGG |
| SEQ ID NO: 241 | GGGGS |
| SEQ ID NO: 242 | GGS |
| SEQ ID NO: 243 | GGGSGGGSGGGS |
| SEQ ID NO: 244 | KKYKPYVPVTTN |
| SEQ ID NO: 245 | EPKSSDKTHT |
| SEQ ID NO: 246 | GGGSGGSGGGS |
| SEQ ID NO: 247 | G |
| SEQ ID NO: 248 | A |
| SEQ ID NO: 249 | GS |
| SEQ ID NO: 250 | GA |
| SEQ ID NO: 251 | GSS |
| SEQ ID NO: 252 | GSSS |
| SEQ ID NO: 253 | GSSSS |
| SEQ ID NO: 254 | (GSSS)_{y} where y = 1 to 10 |
| SEQ ID NO: 255 | VA |
| SEQ ID NO: 256 | VZ |
| SEQ ID NO: 257 | GVZG |
| SEQ ID NO: 258 | FK |
| SEQ ID NO: 259 | VK |
| SEQ ID NO: 260 | GVAG |
| SEQ ID NO: 261 | GVZGG |
| SEQ ID NO: 262 | XG |
| SEQ ID NO: 263 | XGG |
| SEQ ID NO: 264 | XA |
| SEQ ID NO: 265 | XAA |
| SEQ ID NO: 266 | XGS |
| SEQ ID NO: 267 | XGA |
| SEQ ID NO: 268 | XGVZG |
| SEQ ID NO: 269 | XGVAG |
| SEQ ID NO: 270 | XGVZGG |
| SEQ ID NO: 271 | Val-Arg |
| SEQ ID NO: 272 | Val-Cit |
| SEQ ID NO: 273 | Phe-Lys |
| SEQ ID NO: 274 | Met-Lys |
| SEQ ID NO: 275 | Asn-Lys |
| SEQ ID NO: 276 | Ile-Pro |
| SEQ ID NO: 277 | Gly-Ile |
| SEQ ID NO: 278 | Gly-Leu |
| SEQ ID NO: 279 | Gly-Tyr |
| SEQ ID NO: 280 | Gly-Met |
| SEQ ID NO: 281 | Met-Ile |
| SEQ ID NO: 282 | Ala-Ile |
| SEQ ID NO: 283 | Pro-Ile |
| SEQ ID NO: 284 | Gly-Pro-Gln-Gly-Ile-Ala-Gly-Gln |
| SEQ ID NO: 285 | Gly-Pro-Gln-Gly-Ile-Phe-Gly-Gln |
| SEQ ID NO: 286 | Gly-Pro-Gln-Gly-Ile-Trp-Gly-Gln |
| SEQ ID NO: 287 | Gly-Pro-Gln-Gly-Ile-Leu-Gly-Gln |
| SEQ ID NO: 288 | Gly-Pro-Gln-Gly-Ile-Arg-Gly-Gln |
| SEQ ID NO: 289 | Gly-Pro-Leu-Gly-Ile-Ala-Gly-Gln |
| SEQ ID NO: 290 | Gly-Pro-Met-Gly-Ile-Ala-Gly-Gln |
| SEQ ID NO: 291 | Gly-Pro-Tyr-Gly-Ile-Ala-Gly-Gln |
| SEQ ID NO: 292 | GSVAGS |
| SEQ ID NO: 293 | GGGGSVAGGGGS |
| SEQ ID NO: 294 | GGGGSGGGGSVAGGGGSGGGGS |
| SEQ ID NO: 295 | GGGGSGGGGSPLGLAGGGGGSGGGGS |
| SEQ ID NO: 296 | AEAAAKEAAAKAVAAEAAAKEAAAKA |
| SEQ ID NO: 297 | GGGSGGGS |

A linker can provide a minimum distance between the TfR-binding peptide and the oligonucleotide, such that the oligonucleotide does not inhibit or prevent binding of the peptide to TfR. A linker can provide a minimum distance between the TfR-binding peptide and the oligonucleotide, such that the peptide does not inhibit or prevent binding of the oligonucleotide to its pairing target or the approach of necessary protein complexes. A linker can be long enough to avoid steric hindrance of the oligonucleotide inhibiting binding of the peptide to TfR. A linker can be longer than the shortest distance of the N-terminal amine in the peptide to TfR when bound, which can be around 5 angstroms. A linker can hold the oligonucleotide at an adequate distance from TfR so that electrostatic repulsion caused by the negative charge on the oligonucleotide does not inhibit or prevent the peptide from binding TfR. A linker can be longer than a salt bridge, which can be 2-4 angstroms long. A linker can be at least 5, 10, 20, 40 or more angstroms long. A linker can comprise at least 1, at least 2, at least 3, at least 5, at least 10, at least 15, at least 20, and least 25, or more carbon, oxygen, nitrogen, sulfur, and/or phosphorous atoms in the linker backbone between the peptide and the oligonucleotide. A linker can include 1, 2, 3, 4, 5, 10, 15, 20 or more amino acids. A linker can include 1, 2, 3, 4, 5, 10, 20 or more nucleotide bases.

A peptide oligonucleotide complex according to the present disclosure may be attached to another moiety such as a small molecule, a second peptide, a protein, an immune-oncology agent, a cytokine, a cytokine-receptor chain complex, an antibody, an antibody fragment, an aptamer, polypeptide, polynucleotide, a double stranded (ds) DNA or RNA, a single stranded DNA or RNA, a microRNA, a siRNA, a panhandle RNA, a hairpin RNA, a cyclic dinucleotide, a fluorophore, a radioisotope, a radionuclide chelator, a polymer, a biopolymer, a fatty acid, an acyl adduct, a chemical linker, or sugar, immune-oncology agent, or other active agent described herein through a linker, or directly in the absence of a linker.

A peptide or nucleotide of a peptide oligonucleotide complex can be conjugated a nucleotide, an active agent, or a detectable agent via a linker that can be described with the formula Peptide-A-B-C-active agent. A can be a stable amide link to an amine or carboxylic acid on the peptide and the linker and can be achieved via a tetrafluorophenyl (TFP) ester, an NHS ester, or an ATT group (thiazolidine-thione). A can be a stable carbamate linker such as that formed by reacting an amine on the peptide with an imidazole carbamate active intermediate formed by reaction of CDI with a hydroxyl on the linker. A can be a stable secondary amine linkage such as that formed by reductive alkylation of the amine on the peptide with an aldehyde or ketone group on the linker. A can be a stable thioether linker formed using a maleimide or bromoacetamide in the linker with a thiol in the peptide, a triazole linker, a stable oxime linker, or an oxacarboline linker. A can comprise a triazole. B can comprise (—CH2—)ₓ—, with or without branching a short PEG (—CH₂CH₂O—)ₓ (x is 1-20), or a short polypeptide such as GGGSGGGS (SEQ ID NO: 297), Val-Ala (SEQ ID NO: 255), Val-Cit (SEQ ID NO: 272), Val-Cit-PABC, Gly-Ile (SEQ ID NO: 277), Gly-Leu (SEQ ID NO: 278), other spacers, or no spacer. C can be a disulfide bond, an amide bond, a triazole bond, carbamate, a carbon-carbon single double or triple bond, or an ester bond to a thiol, an amine, a hydroxyl, or carboxylic acid on the active agent. C can be a thioether formed between a maleimide on the linker and a sulfhydroyl on the active agent, a secondary or tertiary amine, a carbamate, or other stable bond. In some embodiments, C can refer to the “cleavable” or “stable” part of the linker. In other embodiments, A and/or B can also be the “cleavable” or stable part. In some embodiments, A can be amide, carbamate, thioether via maleimide or bromoacetamide, triazole, oxime, or oxacarboline. Any linker chemistry described in “Current ADC Linker Chemistry,” Jain et al., Pharm Res, 2015 DOI 10.1007/s11095-015-1657-7 or in Bioconjugate Techniques, 3^{rd} edition, by Greg Hermanson can be used.

In some cases, a linker can comprise a triazole group, such as any one of the heterocyclic compounds with molecular formula C₂H₃N₃, having a five-membered ring of two carbon atoms and three nitrogen atoms, optionally with a hydrogen atom bonded to N at any position in the ring, such as:

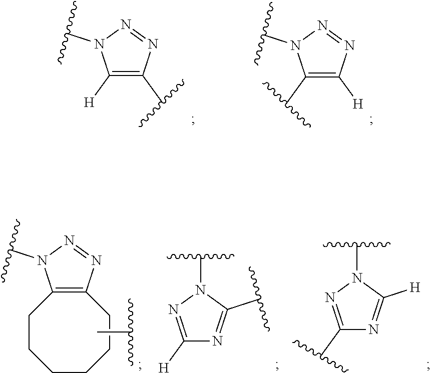

for example, a 1, 2, 3-Triazole (such as 1H1,2,3-Triazole, 2H1,2,3-Triazole, or 1-methyl-4,5,6,7,8,9-hexahydro-1H-cycloocta[d][1,2,3]triazole) or a 1,2,4-Triazole (such as 1H1,2,4-Triazole or 4H1,2,4-Triazole).

Additional non-limiting examples of linkers include linear or non-cyclic linkers such as:

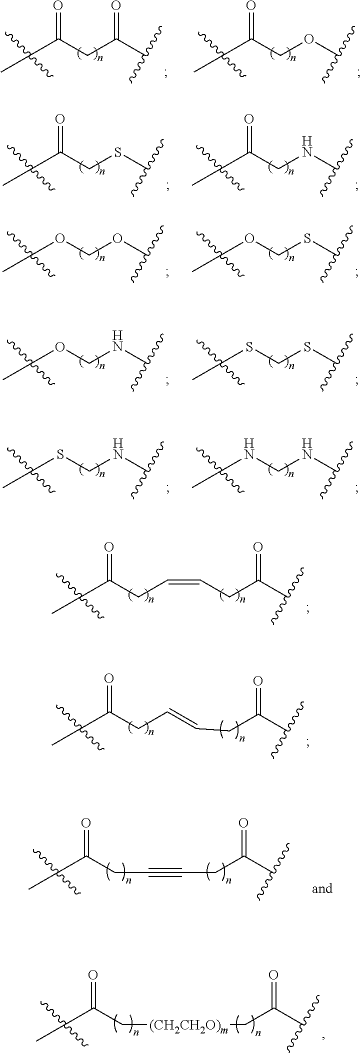

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50, or any linker as disclosed in Jain, N., Pharm Res. 32(11): 3526-40 (2015) or Ducry, L., Antibody Drug Conjugates (2013).

In some cases, a linker can comprise a cyclic group, such as an organic nonaromatic or aromatic ring, optionally with 3-10 carbons in the ring, optionally built from a carboxylic acid,

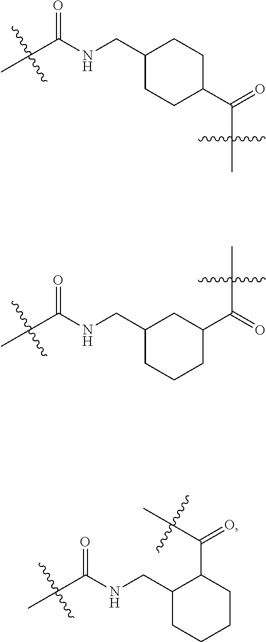 [SMILES: *C(=O)NCC1CCC(C(*)=O)CC1 ; *C(=O)NCC1CCCC(C(*)=O)C1 ; *C(=O)NCC1CCCCC1C(*)=O]

for example trans-4-(aminomethyl) cyclohexane carboxylic acid,

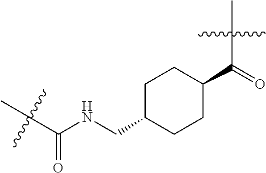 [SMILES: *C(=O)NC[C@H]1CC[C@H](C(*)=O)CC1]

or a substituted analog or a stereoisomer thereof. This linker can optionally be used to form a carbamate linkage. In some cases, a carbamate linkage can be more resistant to cleavage, such as by hydrolysis, enzymes such as esterases, or other chemical reactions, than an ester linkage.

In some cases, a linker can comprise a cyclic carboxylic acid, for example a cyclic dicarboxylic acid, for example one of the following groups: 1,4-cyclohexane dicarboxylic acid, 1,2-cyclohexane dicarboxylic acid, or 1,3-cyclohexane dicarboxylic acid, 1,1-cyclopentanediacetic acid,

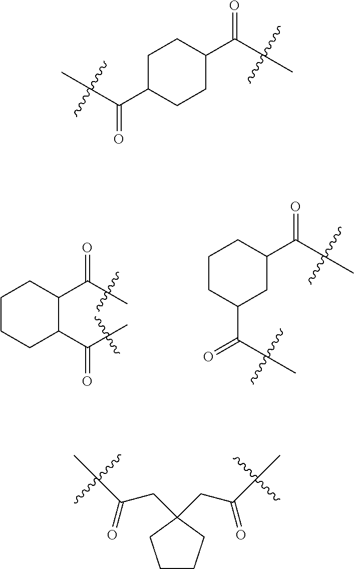 [SMILES: *C(=O)C1CCC(C(*)=O)CC1 ; *C(=O)C1CCCCC1C(*)=O ; *C(=O)C1CCCC(C(*)=O)C1 ; *C(=O)CC1(CC(*)=O)CCCC1]

or a substituted analog or a stereoisomer thereof. For example, the linker can comprise one of the following groups.

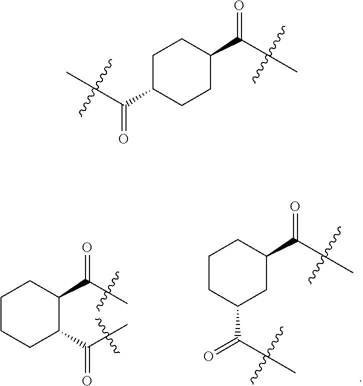 [SMILES: *C(=O)[C@H]1CC[C@H](C(*)=O)CC1 ; *C(=O)[C@@H]1CCCC[C@H]1C(*)=O ; *C(=O)[C@@H]1CCC[C@@H](C(*)=O)C1]

In some instances, the linker can optionally be used to form an ester linkage. In some cases, a cyclic ester linkage can be more sterically resistant to cleavage, such as by hydrolysis by water, enzymes such as esterases, or other chemical reactions, than a noncyclic or linear ester linkage.

In some cases, a linker can comprise an aromatic dicarboxylic acid, for example terephthalic acid, isophthalic acid, phthalic acid

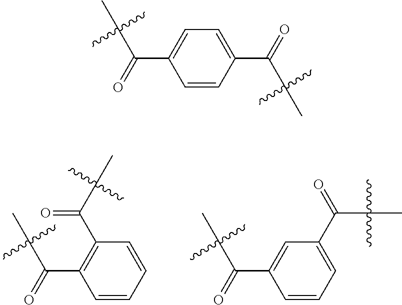 [SMILES: *C(=O)c1ccc(C(*)=O)cc1 ; *C(=O)c1ccccc1C(*)=O ; *C(=O)c1cccc(C(*)=O)c1]

or a substituted analog thereof.

In some cases, a linker can comprise a natural or non-natural amino acid, for example cysteine,

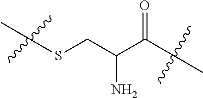 [SMILES: *SCC(N)C(*)=O]

or a substituted analog or a stereoisomer thereof. In some instances, a linker can comprise alanine (A, Ala); arginine (R, Arg); asparagine (N, Asn); aspartic acid (D, Asp); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val); or any plurality or combination thereof. In some embodiments, the non-natural amino acid can comprise one or more functional groups, e.g., alkene or alkyne, that can be used as functional handles.

In some cases, a linker can comprise one of the following groups:

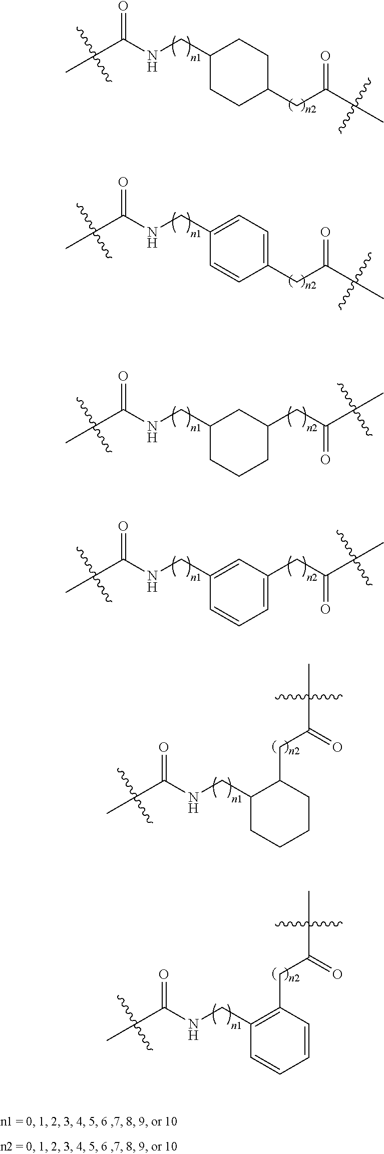

or a substituted analog or a stereoisomer thereof. In some instances, the linker is selected from one of the following groups:

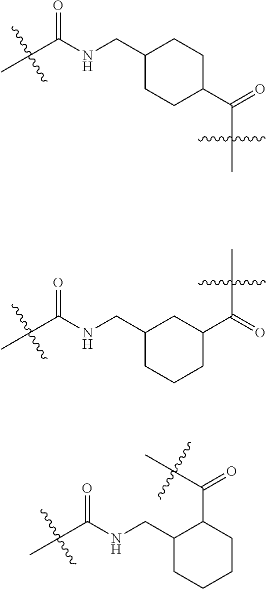 [SMILES: *C(=O)NCC1CCC(C(*)=O)CC1 ; *C(=O)NCC1CCCC(C(*)=O)C1 ; *C(=O)NCC1CCCCC1C(*)=O]

or a substituted analog or a stereoisomer thereof.

In some cases, a linker can comprise one of the following groups:

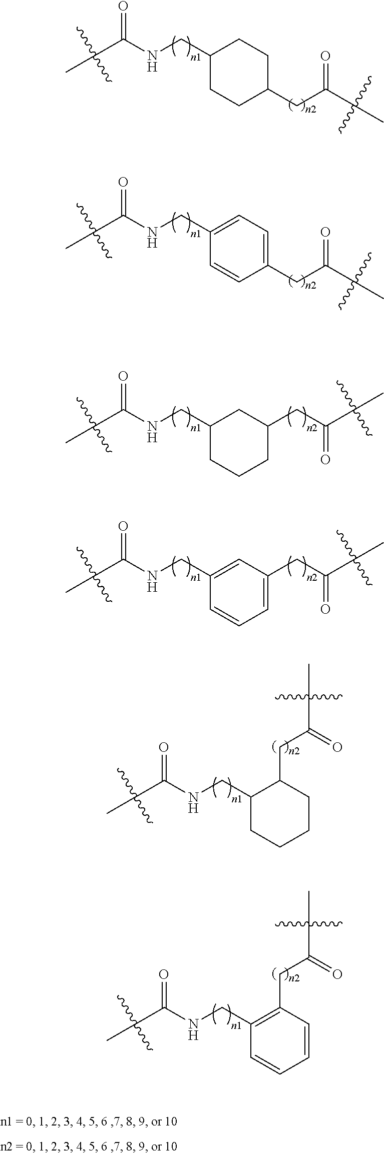

or a substituted analog or a stereoisomer thereof. In some instances, the linker is selected from one of the following groups:

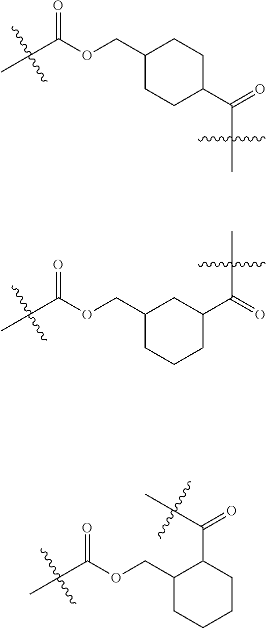 [SMILES: *C(=O)OCC1CCC(C(*)=O)CC1 ; *C(=O)OCC1CCCC(C(*)=O)C1 ; *C(=O)OCC1CCCCC1C(*)=O]

or a substituted analog or a stereoisomer thereof.

In some cases, a substituted analog or a stereoisomer is a structural analog of a compound disclosed herein, for which one or more hydrogen atoms of the compound can be substituted by one or more groups of halo (e.g., Cl, F, Br), alkyl (e.g., methyl, ethyl, propyl), alkenyl, alkynyl, arylalkyl, cycloalkyl, cycloalkylalkyl, heteroalkyl, heterocycloalkyl, or any combination thereof. In some cases, a stereoisomer can be an enantiomer, a diastereomer, a cis or trans stereoisomer, a E or Z stereoisomer, or a R or S stereoisomer.

Non-limiting examples of linear linkers include;

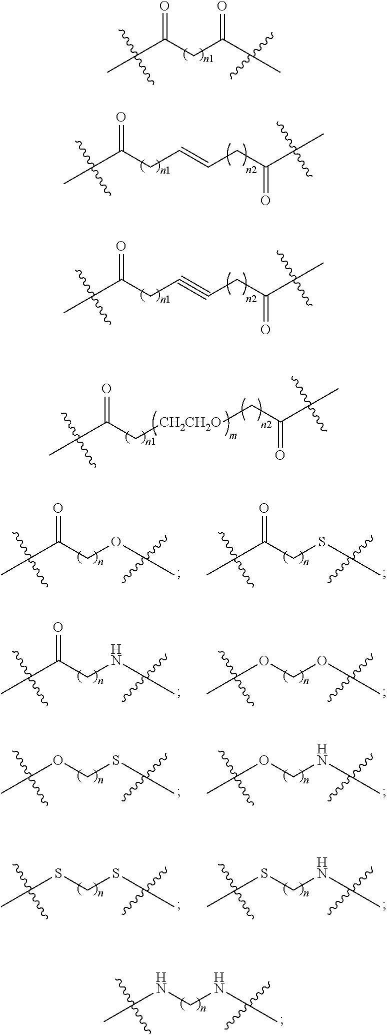

wherein each n1, or n2 or m is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some instances, the linker can comprise a linear dicarboxylic acid, e.g., one of the following groups: succinic acid, 2,3-dimethylsuccinic acid, glutaric acid, adipic acid, 2,5-dimethyladipic acid,

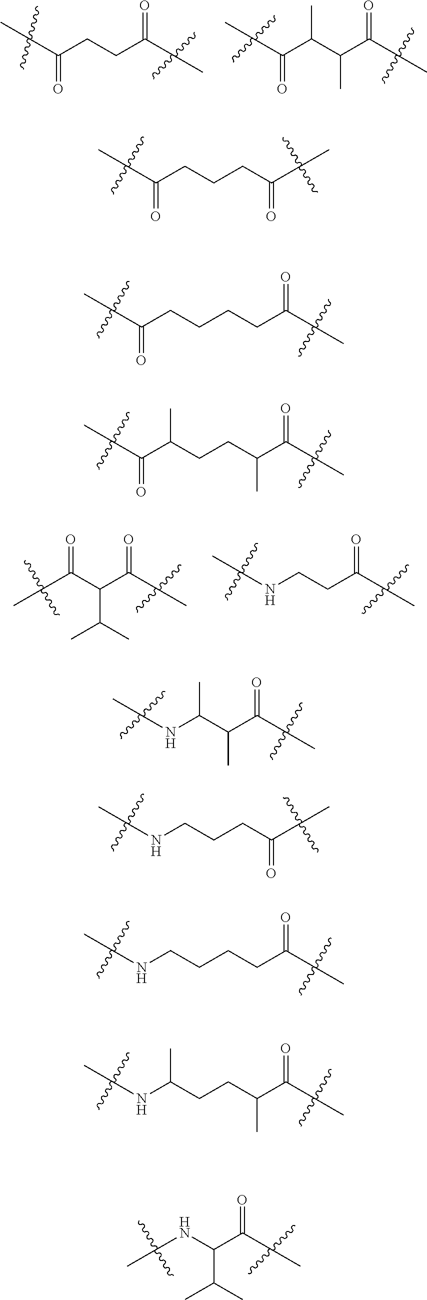

or a substituted analog or a stereoisomer thereof. In some cases, the linker can be used to form a carbamate linkage. In some embodiments, the carbamate linkage can be more resistant to cleavage, such as by hydrolysis, enzymes such as esterases, or other chemical reactions, than an ester linkage. In some cases, the linker can be used to form a linear ester linkage. In some embodiments, the linear ester linkage can be more susceptible to cleavage, such as by hydrolysis, enzymes such as esterases, or other chemical reactions, than a cyclic ester or carbamate linkage. Side chains such as methyl groups on the linear ester linkage can optionally make the linkage less susceptible to cleavage than without the side chains.

In some cases a linker can be a succinic linker, and a targeting agent (e.g., a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter) or other active agent or detectable agent can be attached to a peptide via an ester bond or an amide bond with two methylene carbons in between. In other cases, a linker can be any linker with both a hydroxyl group and a carboxylic acid, such as hydroxy hexanoic acid or lactic acid.

In some cases, a nucleotide (e.g., a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter), an active agent, or a detectable agent can be attached to a peptide using any one or more of the linkers shown below in TABLE 11. In some embodiments, a peptide, an additional active agent, or a detectable agent can be attached to a nucleotide any one or more of the linkers shown below in TABLE 11.

TABLE 11
Exemplary Linkers for Use in Peptide Conjugates or Complexes

| Compound | | |
|---|---|---|
| number | Chemical structure | Linker is based on: |
| 1 | 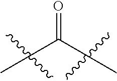 [SMILES: *C(*)=O] | Carbonic acid |
| 2 | 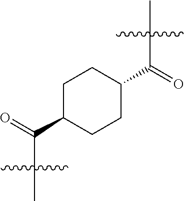 [SMILES: *C(=O)[C@H]1CC[C@H](C(*)=O)CC1] | trans-1,4- cyclohexanedicarboxylic acid |
| 3 | 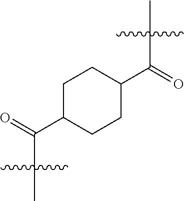 [SMILES: *C(=O)C1CCC(C(*)=O)CC1] | 1,4-cyclohexanedicarboxylic acid |
| 4 | 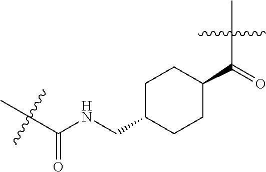 [SMILES: *C(=O)NC[C@H]1CC[C@H](C(*)=O)CC1] | trans-1-aminomethylamine- cyclohexane-4-carboxylic acid (carbamate) |
| 5 | 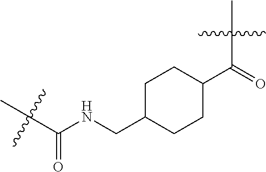 [SMILES: *C(=O)NCC1CCC(C(*)=O)CC1] | 1 -aminomethylamine- cyclohexane-4-carboxylic acid |
| 6 | 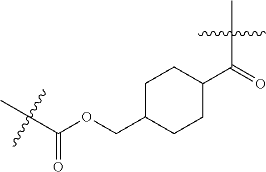 [SMILES: *C(=O)OCC1CCC(C(*)=O)CC1] | 4-(carboxyoxy)-methyl- cyclohexane-1-carboxylic acid |
| 7 | 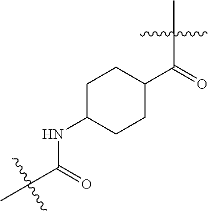 [SMILES: *C(=O)NC1CCC(C(*)=O)CC1] | 4-(carboxyamino)- cyclohexane-1-carboxylic acid |
| 8 | 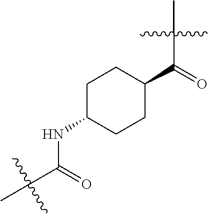 [SMILES: *C(=O)N[C@H]1CC[C@H](C(*)=O)CC1] | trans-4-(carboxyamino)- cyclohexane-1-carboxylic acid |
| 9 | 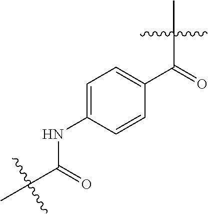 [SMILES: *C(=O)Nc1ccc(C(*)=O)cc1] | 4-(carboxyamino)benzoic acid |
| 10 | 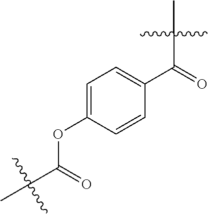 [SMILES: *C(=O)Oc1ccc(C(*)=O)cc1] | 4-(carboxyoxy)benzoic acid |
| 11 | 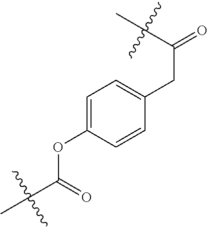 [SMILES: *C(=O)Cc1ccc(OC(*)=O)cc1] | 2-(4- (carboxyoxy)phenyl)acetic acid |
| 12 | 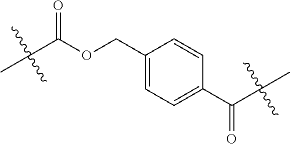 [SMILES: *C(=O)OCc1ccc(C(*)=O)cc1] | 4- ((carboxyoxy)methyl)benzoic acid |
| 13 | 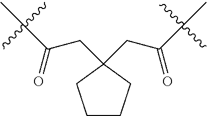 [SMILES: *C(=O)CC1(CC(*)=O)CCCC1] | 2,2′-(cyclopentane-1,1- diyl)diacetic acid |
| 14 | 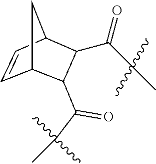 [SMILES: *C(=O)C1C2C=CC(C2)C1C(*)=O] | cis-5-norborene-endo-2,3- dicarboxylic acid |
| 15 | 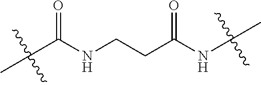 [SMILES: *NC(=O)CCNC(*)=O] | (3-amino-3- oxypropyl)carbamic acid |
| 16 | 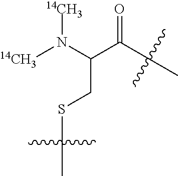 | [¹⁴C]-Cysteine |
| 17 | 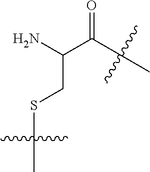 | Cysteine |
| 18 | 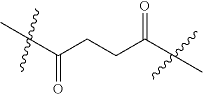 [SMILES: *C(=O)CCC(*)=O] | Succinic acid |
| 19 | 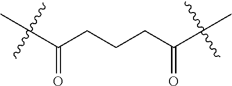 [SMILES: *C(=O)CCCC(*)=O] | Glutaric acid |
| 20 | 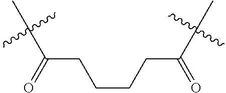 [SMILES: *C(=O)CCCCC(*)=O] | Adipic acid |
| 21 | 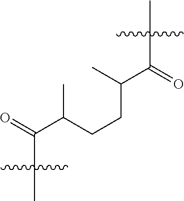 [SMILES: *C(=O)C(C)CCC(C)C(*)=O] | 2,5-dimethyladipic acid (DMA) |
| 22 | 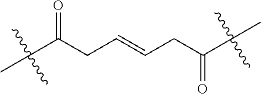 [SMILES: *C(=O)C/C=C/CC(*)=O] | trans-β-hydromuconic acid |
| 23 | 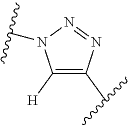 [SMILES: *c1cn(*)nn1] | 1,4-dimethyl-1H-1,2,3-triazole |
| 24 | 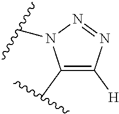 [SMILES: *c1cnnn1*] | 1,5-dimethyl-1H-1,2,3-triazole |
| 25 | 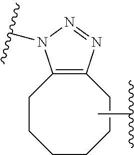 | 1-methyl-4,5,6,7,8,9- hexahydro-1H- cycloocta[d][1,2,3]triazole |
| 26 | 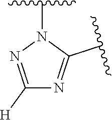 [SMILES: *c1ncnn1*] | 1,5-dimethyl-1H-1,2,4-triazole |
| 27 | 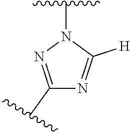 [SMILES: *c1ncn(*)n1] | 1,3-dimethyl-1H-1,2,4-triazole |

In some cases, a targeting agent (e.g., a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-MIR, splice blocker ASO, or U1 Adapter) or other active agent or detectable agent is attached to a linker wherein a nucleophilic functional group (e.g., a hydroxyl group) of the targeting agent molecule acts as the nucleophile and replaces a leaving group on the linker moiety, thereby attaching it to the linker.

In other cases, a targeting agent (e.g., a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter) or other active agent or detectable agent is attached to a linker wherein a nucleophilic functional group (e.g., thiol group, amine group, etc) of the linker replaces a leaving group on the targeting agent, active agent, or detectable agent molecule, thereby attaching it to the linker. Such leaving group (or functional group that may be converted into a leaving group) may be a primary alcohol to form a thioether bond, thereby attaching it to the linker. A primary alcohol can be converted into a leaving group such as a mesylate, a tosylate, or a nosylate in order to accelerate the nucleophilic substitution reaction.

The peptide-active agent conjugates of the present disclosure (e.g., peptide oligonucleotide complexes) can comprise an active agent (e.g., a nucleotide targeting agent, a therapeutic agent, a detectable agent, or a combination thereof), a linker, and/or a peptide of the present disclosure. A general connectivity between these three components can be active agent-linker-peptide, such that the linker is attached to both the active agent and the peptide. In many cases, the peptide is attached to a linker via an amide bond. Amide bonds can be relatively stable (e.g., in vivo) compared to other bonds described herein, such as esters, carbonates, etc. The amide bond between the peptide and the linker may thus provide advantageous properties due to its in vivo stability of the active agent (e.g., the nucleotide targeting agent, the therapeutic agent, or the detectable agent) is sought to be cleaved from a peptide-active agent-conjugate without the linker being attached to the active agent after such in vivo cleavage. Thus, in various cases, an active agent is attached to the linker-peptide moiety via linkages such as ester, carbonate, carbamate, etc., wherein the peptide or active agent is attached to the linker via an amide bond. This can allow for selective cleavage of the active agent-linker bond (as opposed to the linker-peptide bond) allowing the active agent to be released without a linker moiety attached to it after cleavage. The use of such different active agent-linker bonds or linkages can allow the modulation of active agent release in vivo, e.g., in order to achieve a therapeutic function while minimizing off-target effects (e.g., reduction in drug release during circulation).

The linker can be a cleavable or a stable linker. The use of a cleavable linker permits release of the conjugated moiety (e.g., a nucleotide targeting agent, a therapeutic agent, a detectable agent, or a combination thereof) from the peptide, e.g., after targeting to the target tissue or cell or subcellular compartment or after endocytosis. In some cases, the linker is enzyme cleavable, e.g., a valine-citrulline linker that can be cleavable by cathepsin, or an ester linker that can be cleavable by esterase. In some embodiments, the linker contains a self-immolating portion. In other embodiments, the linker includes one or more cleavage sites for a specific protease, such as a cleavage site for matrix metalloproteases (MMPs), thrombin, urokinase-type plasminogen activator, or cathepsin (e.g., cathepsin K).

Thus, in some cases, a peptide-active agent conjugate of the present disclosure can comprise one or more, about two or more, about three or more, about five or more, about ten or more, or about 15 or more amino acids that can form an amino acid sequence cleavable by an enzyme. Such enzymes can include proteinases. A peptide-drug conjugate can comprise an amino acid sequence that can be cleaved by a Cathepsin, a Chymotrypsin, an Elastase, a Subtilisin, a Thrombin I, or a Urokinas, or any combination thereof.

Alternatively or in combination, the cleavable linker can be cleaved, dissociated, or broken by other mechanisms, such as via pH, reduction, or hydrolysis. Hydrolysis can occur directly due to water reaction, or be facilitated by an enzyme, or be facilitated by presence of other chemical species. A hydrolytically labile linker, (amongst other cleavable linkers described herein) can be advantageous in terms of releasing active agents from the peptide. For example, an active agent in a conjugate form with the peptide may not be active, but upon release from the conjugate after targeting to the target tissue or cell or subcellular compartment, the active agent is active. The cleaved active agent may retain the chemical structure of the active agent before cleavage, or may be modified. In some embodiments, a stable linker may optionally not cleave in buffer over extended periods of time (e.g., hours, days, or weeks). In some embodiments, a stable linker may optionally not cleave in body fluids such as plasma or synovial fluid over extended periods of time (e.g., hours, days, or weeks). In some embodiments, a stable linker optionally may cleave, such as after exposure to enzymes, reactive oxygen species, other chemicals or enzymes that may be present in cells (such as macrophages), cellular compartments (such as endosomes and lysosomes), inflamed areas of the body (such as inflamed joints), or tissues or body compartments. In some embodiments, a stable linker may optionally not cleave in vivo but present an active agent that is still active when conjugated to, linked to, or fused to the peptide.

The rate of hydrolysis of the linker (e.g., a linker of a peptide conjugate) can be tuned. For example, the rate of hydrolysis of linkers with unhindered esters may be faster compared to the hydrolysis of linkers with bulky groups next an ester carbonyl. A bulky group can be a methyl group, an ethyl group, a phenyl group, a ring, or an isopropyl group, or any group that provides steric bulk. In another example, the rate of hydrolysis can be faster with hydrophilic groups, such as alcohols, acids, or ethers, or near an ester carbonyl. In another example, hydrophobic groups present as side chains or by having a longer hydrocarbon linker can slow cleavage of the ester. In some embodiments, cleavage of a carbamate group can also be tuned by hindrance, hydrophobicity, and the like. In another example, using a less labile linker, such as a carbamate rather than an ester, can slow the cleavage rate of the linker. In some cases, the steric bulk can be provided by the drug itself, such as by ketorolac when conjugated via its carboxylic acid. The rate of hydrolysis of the linker can be tuned according to the residency time of the conjugate in the target tissue or cell or subcellular compartment, according to how quickly the peptide accumulates in the target tissue or cell or subcellular compartment, or according to the desired time frame for exposure to the active agent in the target tissue or cell or subcellular compartment. For example, when a peptide is cleared from the target tissue or cell or subcellular compartment relatively quickly, the linker can be tuned to rapidly hydrolyze. In contrast, for example, when a peptide has a longer residence time in the target tissue or cell or subcellular compartment, a slower hydrolysis rate can allow for extended delivery of an active agent. This can be important when the peptide is used to deliver a drug to the target tissue or cell or subcellular compartment (e.g., a tumor cell or a tumor tissue). “Programmed hydrolysis in designing paclitaxel prodrug for nanocarrier assembly” Sci Rep 2015, 5, 12023 Fu et al., provides an example of modified hydrolysis rates. In some embodiments, rates of cleavage can vary by species, body compartment, and disease state. For instance, cleavage by esterases may be more rapid in rat or mouse plasma than in human plasma, such as due to different levels of carboxyesterases. In some embodiments, a linker may be tuned for different cleavage rates for similar cleavage rates in different species.

In some cases, a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid.

In some embodiments, the linker can release the active agent in an unmodified form. In other embodiments, the active agent can be released with chemical modification. In still other embodiments, catabolism can release the active agent still linked to parts of the linker and/or peptide.

The linker can be a stable linker or a cleavable linker. In some embodiments, the stable linker can slowly release the conjugated moiety by an exchange of the conjugated moiety onto the free thiols on serum albumin. In some embodiments, the use of a cleavable linker can permit release of the conjugated moiety (e.g., a therapeutic agent) from the peptide, e.g., after administration to a subject in need thereof. In other embodiments, the use of a cleavable linker can permit the release of the conjugated therapeutic from the peptide. In some cases, the linker is enzyme cleavable, e.g., a valine-citrulline linker. In some embodiments, the linker contains a self-immolating portion. In other embodiments, the linker includes one or more cleavage sites for a specific protease, such as a cleavage site for matrix metalloproteases (MMPs), thrombin, cathepsins, peptidases, or beta-glucuronidase. Alternatively or in combination, the linker is cleavable by other mechanisms, such as via pH, reduction, or hydrolysis.

The rate of hydrolysis or reduction of the linker can be fine-tuned or modified depending on an application. For example, the rate of hydrolysis of linkers with unhindered esters can be faster compared to the hydrolysis of linkers with bulky groups next to an ester carbonyl. A bulky group can be a methyl group, an ethyl group, a phenyl group, a ring, or an isopropyl group, or any group that provides steric bulk. In some cases, the steric bulk can be provided by the drug itself, such as by ketorolac when conjugated, linked, or fused via its carboxylic acid. The rate of hydrolysis of the linker can be tuned according to the residency time of the conjugate or fusion in the target location. For example, when a peptide is cleared from a tumor, or the brain, relatively quickly, the linker can be tuned to rapidly hydrolyze. When a peptide has a longer residence time in the target location, a slower hydrolysis rate would allow for extended delivery of an active agent.

The rate of hydrolysis of the linker (e.g., a linker of a peptide conjugate) can be measured. Such measurements can include determining free active agent in plasma, or synovial fluid, or other fluid or tissue of a subject in vivo and/or by incubating a linker or a peptide conjugate comprising a linker of the present disclosure with a buffer (e.g., PBS) or blood plasma from a subject (e.g., rat plasma, human plasma, etc.) or synovial fluid or other fluids or tissues ex vivo. The methods for measuring hydrolysis rates can include taking samples during incubation or after administration and determine free active agent, free peptide, or any other parameter indicate of hydrolysis, including also measuring total peptide, total active agent, or conjugated active agent-peptide. The results of such measurements can then be used to determine a hydrolysis half-life of a given linker or peptide conjugate comprising the linker. A hydrolysis half-life of a linker can differ depending on the plasma or fluid or species or other conditions used to determine such half-life. This can be due to certain enzymes or other compounds present in a certain plasma (e.g., rat plasma). For instance, different fluids (such as plasma or synovial fluid) can contain different amounts of enzymes such as esterases, and these levels of these compounds can also vary depending on species (such as rat versus human) as well as disease state (such as normal versus arthritic).

The conjugates of the present disclosure can be described as having a modular structure comprising various components, wherein each of the components (e.g., peptide, linker, active agent and/or detectable agent) can be selected dependently or independently of any other component. For example, a conjugate for use in the treatment of pain can comprise a TfR-binding peptide of the present disclosure (e.g., those having the amino acid sequence of any one of SEQ ID NO: 1-SEQ ID NO: 134 or SEQ ID NO: 306-SEQ ID NO: 335), a linker (e.g., any linker described in TABLE 10 or TABLE 11, SEQ ID NO: 234-SEQ ID NO: 297, or otherwise described) and an active agent (e.g., an oligonucleotide targeting a gene related to pain, an NSAID pain reliever, ibuprofen, or a molecule to treat disorders of the CNS or of cancer). The linker, for example, can be selected and/or modified to achieve a certain active agent release (e.g., a certain release rate) via a certain mechanism (e.g., via hydrolysis, such as enzyme and/or pH-dependent hydrolysis) at the target site (e.g., in the brain) and/or to minimize systemic exposure to the active agent. During the testing of a conjugate any one or more of the components of the conjugate can be modified and/or altered to achieve certain in vivo properties of the conjugate, e.g., pharmacokinetic (e.g., clearance time, bioavailability, uptake and retention in various organs) and/or pharmacodynamic (e.g., target engagement) properties. Thus, the conjugates of the present disclosure can be modulated to prevent, treat, and/or diagnose a variety of diseases and conditions, while reducing side effects (e.g., side effects that occur if such active agents are administered alone (i.e., not conjugated to a peptide)).

In some embodiments, the non-natural amino acid can comprise one or more functional groups, e.g., alkene or alkyne, that can be used as functional handles. For example, a multiple bond of such functional groups can be used to add one or more molecules to the conjugate. The one or more molecules can be added using various synthetic strategies, some of which may include addition and/or substitution chemistries. For example, an addition reaction using a multiple bond can comprise the use of hydrobromic acid, wherein the bromine can act as a leaving group and thus be substituted with various moieties, e.g., active agents, detectable agents, agents that can modify or alter the pharmacokinetic (e.g., plasma half-life, retention and/or uptake in central nervous system (CNS) or elsewhere) and/or pharmacodynamic (e.g., hydrolysis rate such as an enzymatic hydrolysis rate) properties of the conjugate.

In some embodiments, a conjugate as described herein comprises one or more non-natural amino acid and/or one or more linkers. Such one or more non-natural amino acid and/or one or more linkers can comprise one or more functional groups, e.g., alkene or alkyne (e.g., non-terminal alkenes and alkynes), which can be used as functional handles. For example, a multiple bond of such functional groups can be used to add one or more molecules to the conjugate. The one or more molecules can be added using various synthetic strategies, some of which may include addition and/or substitution chemistries, cycloadditions, etc. For example, an addition reaction using a multiple bond can comprise the use of hydrogen bromide (e.g., via hydrohalogenation reactions), wherein the bromide substituent, once attached, can act as a leaving group and thus be substituted with various moieties comprising a nucleophilic functional groups, e.g., active agents, detectable agents, agents. As another example, a multiple bond can be used as a functional handle in a cycloaddition reaction. Cycloaddition reactions can comprise 1,3-dipolar cycloadditions, [2+2]-cycloadditions (e.g., photocatalyzed), Diels-Alder reactions, Huisgen cycloadditions, nitrone-olefin cycloadditions, etc. Such cycloaddition reactions can be used to attached various functional groups, functional moieties, active agents, detectable agents, and so forth to the conjugate. For example, a 1,3-dipolar cycloaddition reaction can be used to attach a molecule to a conjugate, wherein the molecule comprises a 1,3-dipolar that can react with, e.g., an alkyne to form a 5-membered ring, thereby attaching said molecule to the conjugate.

The addition of such agents or molecules (e.g., via nucleophilic or electrophilic addition followed by nucleophilic substitution) can have various application. For example, attaching such molecule or agent can modify or alter the pharmacokinetic (e.g., plasma half-life, retention and/or uptake in CNS or biodistribution) and/or pharmacodynamic (e.g., hydrolysis rate such as an enzymatic hydrolysis rate) properties of the conjugate. Attaching such molecule or agent can also alter (e.g., increase) the depot effect of a conjugate, or provide functionality for in vivo tracking, e.g., using fluorescence or other types of detectable agents.

In some embodiments, a conjugate of the present disclosure can comprise a linker comprising one or more of the following groups:

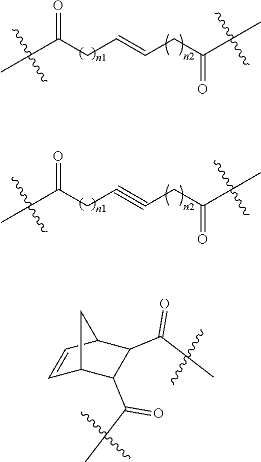

or a substituted analog or a stereoisomer thereof, wherein each n1 and n2 is independently a value from 1 to 10. Such a group can be used as a handle to attach one or more molecules to a conjugate, e.g., to alter the pharmacokinetic (e.g., plasma half-life, retention and/or uptake in central nervous system (CNS) or elsewhere) and/or pharmacodyna via nucleophilic or electrophilic addition followed by nucleophilic substitution mic properties of the conjugate. Functionalization of such a group can occur using one or more multiple bonds (e.g., double bonds, triple bonds, etc.) of the groups. Such functionalization can comprise addition and/or substitution chemistries. For example, a functional group of a linker, such as a double bond, can be converted into a single bond (e.g., via an addition reaction such as a nucleophilic addition reaction), wherein one or both of the carbon atoms of the newly formed single bond can have a leaving group (e.g., a bromine) attached to them. Such a leaving group can then be used (e.g., via nucleophilic substitution reaction) to attach a specific molecule (e.g., an active agent, a detectable agent, etc.) to that carbon atom(s) of the linker.

As another example, a multiple bond can be used as a functional handle in a cycloaddition reaction. Cycloaddition reactions can comprise 1,3-dipolar cycloadditions, [2+2]-cycloadditions (e.g., photocatalyzed), Diels-Alder reactions, Huisgen cycloadditions, nitrone-olefin cycloadditions, etc. Such cycloaddition reactions can be used to attached various functional groups, functional moieties, active agents, detectable agents, and so forth to the conjugate. For example, a 1,3-dipolar cycloaddition reaction can be used to attach a molecule to a conjugate, wherein the molecule comprises a 1,3-dipolar that can react with, e.g., an alkyne to form a 5-membered ring, thereby attaching said molecule (e.g., active agent, detectable agent, etc.) to the conjugate. In some cases, molecules may be attached to a conjugate to e.g., modulate the half-life, increase the depot effect, or provide new functionality of a conjugate, such as fluorescence for tracking.

### Conjugation, Linkers, and Methods of Manufacture

The peptide in the peptide nucleotide complexes described herein can be attached to a nucleotide (e.g., a targeting nucleic acid), for example at the 3′ end of the oligonucleotide, the 5′ end of the oligonucleotide, or on one of the residues within the sequence, including a modified nucleotide base or 3′ or 5′ end with a chemical handle such as an amine, sulfhydryl, azide, alkyne, carboxylic acid, or any other functional group. The peptide can also be attached to a cyclic dinucleotide, such as to a nitrogen, oxygen, carbon, or sulfur atom of the cyclic dinucleotide. The bond can also include a thioester bond, a phosphoester bond, a phosphodiester bond, dithioanalogs of phosphodiester bonds.

The peptide can also be synthesized to contain an azide group, such as an azidohexanoic acid, azidonorleucine, azidohomoalanine, azidoalanine, azidolysine, azidolphenylalanine, azidoornithine; the azidohexanoic acid can be on the N-terminus of the peptide. The peptide can be synthesized to contain an alkyne group, such as homopropargyl glycine, propargyl glycine, or propargyloxyphenylalanine. The nucleotide (e.g., a DNA or RNA polynucleotide or oligonucleotide) can be synthesized to contain, or have appended a linker, that contains an alkyne or an azide, including a alkyne-containing strained cyclooctane ring including those with multiple fused rings such as DIFO, DIBO, DIBAC, BARAC, DIMAC, BCN, OCT, MOFO DIFO2, DIFO3, a Sondheimer diyne, TMDIBO, COBO, S-DIBO, and PYRROC. The peptide-oligonucleotide conjugate can be linked together using a strain-promoted or copper-catalyzed azide-alkyne cycloaddition to create a triazole bond.

The rate of cleavage around a cleavable bond can be varied by varying the local environment around the bond, including carbon length (—CH2—)x, steric hindrance or lack thereof (including adjacent side groups such as methyl, ethyl, cyclic as well as adjacent spacers such as peptidic spacers, which can comprise amino acids such as G, A, or S), hydrophilicity (such as adding hydroxyl, carboxylic acid, or oligoethylene glycol groups), or hydrophobicity (such as adding fluorines, hydrocarbon groups, or fatty tails), adding electron withdrawing or electron donating groups. In some embodiments, cleavage rate can be affected by local pH.

In some embodiments, a nucleotide comprises at least one phosphorothioate linkage. In some embodiments, a peptide oligonucleotide complex comprises from 1 to 12 phosphorothioate linkages. In some embodiments, a nucleotide comprises at least one thiophosphoroamidate linkage. In some embodiments, a nucleotide comprises from 1 to 12 thiophosphoroamidate linkages. In some embodiments, a nucleotide comprises at least one modified base. In some embodiments, at least modified base comprises a 2′F base, an LNA base, a BNA base, an ENA base, a 2′O-MOE base, a 5′-Me base, a (S)-cEt base, a 2′OMe base, a morpholino base, or combinations thereof.

### Pharmacokinetics of Peptide Oligonucleotide Complexes

The pharmacokinetics of any of the peptide oligonucleotide complexes of the present disclosure can be determined after administration of the peptide oligonucleotide complex via different routes of administration. For example, the pharmacokinetic parameters of a peptide oligonucleotide complex of this disclosure can be quantified after intravenous, subcutaneous, intramuscular, rectal, suppository, aerosol, parenteral, ophthalmic, pulmonary, transdermal, vaginal, optic, nasal, oral, sublingual, inhalation, dermal, intrathecal, intranasal, peritoneal, buccal, synovial, intratumoral, intravitreal, or topical administration. Peptide oligonucleotide complexes of the present disclosure can be analyzed by using tracking agents such as radiolabels or fluorophores. For example, radiolabeled peptide oligonucleotide complexes of this disclosure can be administered via various routes of administration. Peptide oligonucleotide complex concentration or dose recovery in various biological samples such as plasma, urine, feces, any organ, skin, muscle, and other tissues can be determined using a range of methods including HPLC, fluorescence detection techniques (TECAN quantification, flow cytometry, iVIS), or liquid scintillation counting.

The methods and compositions described herein relate to pharmacokinetics of peptide oligonucleotide complex administration via any route to a subject. Pharmacokinetics can be described using methods and models, for example, compartmental models or noncompartmental methods. Compartmental models include but are not limited to monocompartmental model, the two compartmental model, the multicompartmental model or the like. Models are often divided into different compartments and can be described by the corresponding scheme. For example, one scheme is the absorption, distribution, metabolism and excretion (ADME) scheme. For another example, another scheme is the liberation, absorption, distribution, metabolism and excretion (LADME) scheme. In some aspects, metabolism and excretion can be grouped into one compartment referred to as the elimination compartment. For example, liberation includes liberation of the active portion of the composition from the delivery system, absorption includes absorption of the active portion of the composition by the subject, distribution includes distribution of the composition through the blood plasma and to different tissues, metabolism, which includes metabolism or inactivation of the composition and finally excretion, which includes excretion or elimination of the composition or the products of metabolism of the composition. Compositions administered intravenously to a subject can be subject to multiphasic pharmacokinetic profiles, which can include but are not limited to aspects of tissue distribution and metabolism/excretion. As such, the decrease in plasma or serum concentration of the composition is often biphasic, including, for example an alpha phase and a beta phase, occasionally a gamma, delta or other phase is observed.

Pharmacokinetics includes determining at least one parameter associated with administration of a peptide oligonucleotide complex to a subject. In some aspects, parameters include at least the dose (D), dosing interval (x), area under curve (AUC), maximum concentration (Cₘₐₓ), minimum concentration reached before a subsequent dose is administered (Cₘᵢₙ), minimum time (Tₘᵢₙ), maximum time to reach Cₘₐₓ (Tₘₐₓ), volume of distribution (V_{d}), steady-state volume of distribution (Vₛₛ), back-extrapolated concentration at time 0 (C₀), steady state concentration (Cₛₛ), elimination rate constant (kₑ), infusion rate (kᵢₙ), clearance (CL), bioavailability (f), fluctuation (% PTF) and elimination half-life (t_{1/2}).

In certain embodiments, the peptide oligonucleotide complexes comprising a peptide of any of SEQ ID NO: 1-SEQ ID NO: 134 or SEQ ID NO: 306-SEQ ID NO: 335 exhibit optimal pharmacokinetic parameters after oral administration. In other embodiments, the peptide oligonucleotide complexes comprising a peptide of any of SEQ ID NO: 1-SEQ ID NO: 134 or SEQ ID NO: 306-SEQ ID NO: 335 exhibit optimal pharmacokinetic parameters after any route of administration, such as oral administration, inhalation, intranasal administration, topical administration, intravenous administration, subcutaneous administration, intra-articular administration, intramuscular administration, intraperitoneal administration, intrα-synovial, intrathecal, intravitreal, intratumoral, or any combination thereof.

In some embodiments, any peptide nucleotide complex comprising a peptide of SEQ ID NO: 1-SEQ ID NO: 134 or SEQ ID NO: 306-SEQ ID NO: 335 exhibits an average Tₘₐₓ of 0.05-12 hours, or 1-48 hours at which the Cₘₐₓ is reached, an average bioavailability in serum of 0.1%-10% in the subject after administering the peptide to the subject by an oral route, an average bioavailability in serum of less than 0.1% after oral administration to a subject for delivery to the GI tract, an average bioavailability in serum of 1-100% after parenteral administration, an average t_{1/2} of 0.1 hours-168 hours, or 0.25 hours-48 hours in a subject after administering the peptide to the subject, an average clearance (CL) of 0.5-100 L/hour or 0.5-50 L/hour of the peptide after administering the peptide to a subject, an average volume of distribution (V_{d}) of 200-20,000 mL in the subject after systemically administering the peptide to the subject, or optionally no systemic uptake, any combination thereof.

### Peptide Stability

A peptide oligonucleotide complex of the present disclosure can be stable in various biological or physiological conditions, such as the pH or reducing environments inside a cell, in the cytosol, in a cell nucleus, or endosome or a tumor. For example, any peptide oligonucleotide complex comprising a peptide of SEQ ID NO: 1-SEQ ID NO: 134 or SEQ ID NO: 306-SEQ ID NO: 335 can exhibit resistance to reducing agents, proteases, oxidative conditions, or acidic conditions.

In some cases, biologic molecules (such as peptides, proteins, and nucleotides) can provide therapeutic functions, but such therapeutic functions are decreased or impeded by instability caused by the in vivo environment. (Moroz et al. Adv Drug Deliv Rev101:108-21 (2016), Mitragotri et al. Nat Rev Drug Discov13(9):655-72 (2014), Bruno et al. Ther Deliv (11):1443-67 (2013), Sinha et al. Crit Rev Ther Drug Carrier Syst. 24(1):63-92 (2007), Hamman et al. BioDrugs 19(3):165-77 (2005)). For instance, the GI tract can contain a region of low pH (e.g., pH˜1), a reducing environment, or a protease-rich environment that can degrade peptides and proteins. Proteolytic activity in other areas of the body, such as the mouth, eye, lung, intranasal cavity, joint, skin, vaginal tract, mucous membranes, and serum, can also be an obstacle to the delivery of functionally active peptides and polypeptides. Additionally, the half-life of peptides in serum can be very short, in part due to proteases, such that the peptide can be degraded too quickly to have a lasting therapeutic effect when administering reasonable dosing regimens. Likewise, proteolytic activity in cellular compartments such as lysosomes and reduction activity in lysosomes and the cytosol can degrade peptides and proteins such that they may be unable to provide a therapeutic function on intracellular targets. Therefore, peptides that are resistant to reducing agents, proteases, and low pH may be able to provide enhanced therapeutic effects or enhance the therapeutic efficacy of co-formulated or conjugated, linked, or fused active agents in vivo.

Additionally, oral delivery of active agents (e.g., nucleotide targeting agents, therapeutic agents, or detectable agents) can be desirable in order to target certain areas of the body (e.g., disease in the GI tract such as colon cancer, irritable bowel disorder, infections, metabolic disorders, and constipation) despite the obstacles to the delivery of functionally active peptides and polypeptides presented by this method of administration. For example, oral delivery of active agents can increase compliance by providing a dosage form that is more convenient for patients to take as compared to parenteral delivery. Oral delivery can be useful in treatment regimens that have a large therapeutic window. Therefore, peptides that are resistant to reducing agents, proteases, and low pH can allow for oral delivery of peptides without nullifying their therapeutic function.

Peptide Resistance to Reducing Agents. Peptides of this disclosure (e.g., a TfR-binding peptide within a peptide oligonucleotide complex) can contain one or more cysteines, which can participate in disulfide bridges that can be integral to preserving the folded state of the peptide. Exposure of peptides to biological environments with reducing agents can result in unfolding of the peptide and loss of functionality and bioactivity. For example, glutathione (GSH) is a reducing agent that can be present in many areas of the body and in cells and can reduce disulfide bonds. As another example, a peptide can become reduced during trafficking of a peptide across the gastrointestinal epithelium after oral administration. A peptide can become reduced upon exposure to various parts of the GI tract. The GI tract can be a reducing environment, which can inhibit the ability of therapeutic molecules with disulfide bonds to have optimal therapeutic efficacy, due to reduction of the disulfide bonds. A peptide can also be reduced upon entry into a cell, such as after internalization by endosomes or lysosomes or into the cytosol, or other cellular compartments. Reduction of the disulfide bonds and unfolding of the peptide can lead to loss of functionality or affect key pharmacokinetic parameters such as bioavailability, peak plasma concentration, bioactivity, and half-life. Reduction of the disulfide bonds can also lead to loss of functionality due to increased susceptibility of the peptide to subsequent degradation by proteases, resulting in rapid loss of intact peptide after administration. In some embodiments, a peptide that is resistant to reduction can remain intact and can impart a functional activity for a longer period of time in various compartments of the body and in cells, as compared to a peptide that is more readily reduced.

In certain embodiments, the peptides of this disclosure can be analyzed for the characteristic of resistance to reducing agents to identify stable peptides. In some embodiments, the peptides of this disclosure can remain intact after being exposed to different molarities of reducing agents such as 0.00001 M-0.0001 M, 0.0001 M-0.001 M, 0.001 M-0.01 M, 0.01 M-0.05 M, 0.05 M-0.1 M, for 15 minutes or more. In some embodiments, the reducing agent used to determine peptide stability can be dithiothreitol (DTT), Tris(2-carboxyethyl)phosphine HCl (TCEP), 2-Mercaptoethanol, (reduced) glutathione (GSH), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a reducing agent. In some embodiments, peptides are completely resistant to GSH reducing conditions and are partially resistant to degradation in DTT reducing conditions. In some embodiments, peptides described herein can withstand or are resistant to degradation in physiological reducing conditions.

Peptide Resistance to Proteases. The stability of peptides of this disclosure can be determined by resistance to degradation by proteases. Proteases, also referred to as peptidases or proteinases, are enzymes that can degrade peptides and proteins by breaking bonds between adjacent amino acids. Families of proteases with specificity for targeting specific amino acids can include serine proteases, cysteine proteases, threonine proteases, aspartic proteases, glutamic proteases, and asparagine proteases. Additionally, metalloproteases, matrix metalloproteases, elastase, carboxypeptidases, Cytochrome P450 enzymes, and cathepsins can also digest peptides and proteins. Proteases can be present at high concentration in blood, in mucous membranes, lungs, skin, the GI tract, the mouth, nose, eye, and in compartments of the cell. Misregulation of proteases can also be present in various diseases such as rheumatoid arthritis and other immune disorders. Degradation by proteases can reduce bioavailability, biodistribution, half-life, and bioactivity of therapeutic molecules such that they are unable to perform their therapeutic function. In some embodiments, peptides that are resistant to proteases can better provide therapeutic activity at reasonably tolerated concentrations in vivo.

In some embodiments, peptides of this disclosure can resist degradation by any class of protease. In certain embodiments, peptides of this disclosure resist degradation by pepsin (which can be found in the stomach), trypsin (which can be found in the duodenum), serum proteases, or any combination thereof. In some embodiments, the proteases used to determine peptide stability can be pepsin, trypsin, chymotrypsin, or any combination thereof. In certain embodiments, peptides of this disclosure can resist degradation by lung proteases (e.g., serine, cysteinyl, and aspartyl proteases, metalloproteases, neutrophil elastase, alpha-1 antitrypsin, secretory leucoprotease inhibitor, and elafin), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a protease.

In some embodiments, from 5% to 10%, from 10% to 20%, from 20% to 30%, from 30% to 40%, from 40% to 50%, from 50% to 60%, from 60% to 70%, from 70% to 80%, from 80% to 90%, or from 90% to 100% of the peptide or the peptide oligonucleotide complex remains intact after exposure to serum, or a solution comprising a peptidase, pepsin, trypsin, or chymotrypsin. Exposure to peptidase, serum, tissue, cell, or other relevant condition, stability of the peptide or the peptide oligonucleotide complexes may be shown when exposed to the serum or enzyme-containing solution, or other relevant condition, relative to a control using a buffer like PBS that does not contain enzyme or serum. Alternatively, exposure to peptidase, serum, tissue, cell, or other relevant condition, stability of peptide or the peptide oligonucleotide complexes may be shown when exposed to the serum or enzyme-containing solution, or other relevant condition, relative to a control using peptides or peptide oligonucleotide complexes comprising the primary (unmodified) peptide sequence exposed to comparable conditions which would be expected to degrade the primary (unmodified) peptide. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide or the peptide oligonucleotide complex remains intact after incubation in human serum at 37° C. for up to 5 min, 15 min, 30 min, 45 min, 1 hour, 2 hours, 4 hours, 8 hours, 12 hours, or 24 hours.

Peptide Stability in Acidic Conditions. Peptides of this disclosure can be administered in biological environments that are acidic. For example, after oral administration, peptides can experience acidic environmental conditions in the gastric fluids of the stomach and gastrointestinal (GI) tract. The pH of the stomach can range from about 1-4 and the pH of the GI tract ranges from acidic to normal physiological pH descending from the upper GI tract to the colon. In addition, the vagina, late endosomes, and lysosomes can also have acidic pH values, such as less than pH 7. These acidic conditions can lead to denaturation of peptides and proteins into unfolded states. Unfolding of peptides and proteins can lead to increased susceptibility to subsequent digestion by other enzymes as well as loss of biological activity of the peptide. In certain embodiments, the peptides of this disclosure can resist denaturation and degradation in acidic conditions and in buffers, which simulate acidic conditions. In certain embodiments, peptides of this disclosure can resist denaturation or degradation in buffer with a pH less than 1, a pH less than 2, a pH less than 3, a pH less than 4, a pH less than 5, a pH less than 6, a pH less than 7, or a pH less than 8. In some embodiments, peptides of this disclosure remain intact at a pH of 1-3. In certain embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH less than 1, a pH less than 2, a pH less than 3, a pH less than 4, a pH less than 5, a pH less than 6, a pH less than 7, or a pH less than 8. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH of 1-3. In other embodiments, the peptides of this disclosure can be resistant to denaturation or degradation in simulated gastric fluid (pH 1-2). In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to simulated gastric fluid. In some embodiments, low pH solutions such as simulated gastric fluid can be used to determine peptide stability.

In some embodiments, the peptides described herein are resistant to degradation in vivo, in the serum of a subject, or inside a cell. In some embodiments, the peptides are stable at physiological pH ranges, such as about pH 7, about pH 7.5, between about pH 5 to 7.5, between about 6.5 to 7.5, between about pH 5 to 8, or between about pH 5 to 7. In some embodiments, the peptides described herein are stable in acidic conditions, such as less than or equal to about pH 5, less than or equal to about pH 3, or within a range from about 3 to about 5. In some embodiments, the peptides are stable in conditions of an endosome or lysosome, or inside a nucleus.

Peptide Stability at High Temperatures. Peptides of this disclosure can be administered in biological environments with high temperatures. For example, after oral administration, peptides can experience high temperatures in the body. Body temperature can range from 36° C. to 40° C. High temperatures can lead to denaturation of peptides and proteins into unfolded states. Unfolding of peptides and proteins can lead to increased susceptibility to subsequent digestion by other enzymes as well as loss of biological activity of the peptide. In some embodiments, a peptide of this disclosure can remain intact at temperatures from 25° C. to 100° C. High temperatures can lead to faster degradation of peptides. Stability at a higher temperature can allow for storage of the peptide in tropical environments or areas where access to refrigeration is limited. In certain embodiments, 5%-100% of the peptide can remain intact after exposure to 25° C. for 6 months to 5 years. 5%-100% of a peptide can remain intact after exposure to 70° C. for 15 minutes to 1 hour. 5%-100% of a peptide can remain intact after exposure to 100° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 25° C. for at least 6 months to 5 years. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 70° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 100° C. for 15 minutes to 1 hour.

Oligonucleotide Resistance to Nucleases. The stability of oligonucleotide target-binding agents of this disclosure can be determined by resistance to degradation by nucleases. Incorporation of modified nucleotides into the oligonucleotide offers advantages, such as the increased stability against nuclease degradation, improved affinities, expanded chemical functionality, and increased molecular diversity. Nucleases, also referred to as hydrolases, are enzymes that can degrade DNA and or RNA by hydrolyzing phosphoric acid ester in the nucleic acid backbone. Families of nucleases with specificity for targeting DNA or RNA can include exonucleases, endonucleases, DNases or RNases, topoisomerases, recombinases, ribozymes, or RNA splicing enzymes. Additionally, nucleases can be protein or RNA and use water, (deoxy)ribose, inorganic phosphate, or the sidechains of Ser, Tyr or His as a nucleophile. Catalysis may or may not require metal ions. In some embodiments, oligonucleotide target-binding agents that are resistant to nucleases can better provide therapeutic activity at reasonably tolerated concentrations in vivo.

In some embodiments, oligonucleotide target-binding agents of this disclosure or or peptide oligonucleotide complexes can resist degradation by any class of nuclease. In certain embodiments, oligonucleotide target-binding agents of this disclosure or peptide oligonucleotide complexes resist degradation by exonucleases, endonucleases, DNases or RNases, topoisomerases, recombinases, ribozymes, or RNA splicing enzymes or any combination thereof. In certain embodiments, oligonucleotide target-binding agents of this disclosure or peptide oligonucleotide complexes can resist degradation by nucleases in target tissues and cells, and as present in modes of delivery including, for example, in serum, intravenous, subcutaneous, intramuscular, rectal, suppository, aerosol, parenteral, ophthalmic, pulmonary, transdermal, vaginal, optic, nasal, oral, sublingual, inhalation, dermal, intrathecal, intratumoral, intranasal, intravitreal, intravesical (such as by administration through a catheter to the bladder), and topical administration, or any combination thereof. In some embodiments, at least about 5%, at least about 10%, at least about 20%, at least about 30%, at least about 40%, at least about 50%, at least about 60%, at least about 70%, at least about 80%, at least about 90%, or about 100% of the oligonucleotide or peptide oligonucleotide complex remains intact after exposure to a nuclease. In some embodiments, from 5% to 10%, from 10% to 20%, from 20% to 30%, from 30% to 40%, from 40% to 50%, from 50% to 60%, from 60% to 70%, from 70% to 80%, from 80% to 90%, or from 90% to 100% of the oligonucleotide or peptide oligonucleotide complex remains intact after exposure to a nuclease. Alternatively, to determine stability in serum, the oligonucleotides or peptide oligonucleotide complexes can be mixed in serum and incubated for 30 m at 37° C. and analyzed by RP-HPLC.

In some embodiments, from 5% to 10%, from 10% to 20%, from 20% to 30%, from 30% to 40%, from 40% to 50%, from 50% to 60%, from 60% to 70%, from 70% to 80%, from 80% to 90%, or from 90% to 100% of the nucleotide or the peptide oligonucleotide complex remains intact after exposure to serum, or a solution comprising a nuclease. Exposure to nuclease, serum, tissue, cell, or other relevant condition, stability of the nucleotide or the peptide oligonucleotide complexes may be shown when exposed to the serum or nuclease-containing solution, or other relevant condition, relative to a control using a buffer like PBS that does not contain nuclease or serum. Alternatively, Exposure to nuclease, serum, tissue, cell, or other relevant condition, stability of the nucleotide or the peptide oligonucleotide complexes may be shown when exposed to the serum or nuclease-containing solution, or other relevant condition, relative to a control using nucleotides or peptide oligonucleotide complexes comprising the primary (unmodified) nucleotide sequence exposed to comparable conditions which would be expected to degrade the primary (unmodified) nucleotide. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the nucleotide or the peptide oligonucleotide complex remains intact after incubation in human serum at 37° C. for up to 5 min, 15 min, 30 min, 45 min, 1 hour, 2 hours, 4 hours, 8 hours, 12 hours, or 24 hours.

### Methods

Peptide Production and Purification. In some embodiments, amino acid sequences that are part of a peptide library are synthesized using expression vectors or solid phase or solution phase peptide synthesis methods. For example, TfR-binding peptides can be cloned into a secreted, soluble protein production/expression vector and subsequently be purified. Peptide purification methods include, but are not limited to, affinity purification columns, ion exchange (cation and/or anion column chromatography), reversed-phase chromatography, hydrophobic interaction chromatography, and size exclusion column chromatography. SDS-PAGE followed by Coomassie staining and reverse phase high-pressure liquid chromatography (HPLC) can be used to analyze a sample of the purified protein. Protein concentrations were determined by UV spectral absorption (e.g., absorbance at 280 nm) and/or amino acid analysis. In some embodiments, the peptides may be produced and purified using the methods described in Crook Z R et al. Methods Mol Biol. 2020; 2070:363-396.

Fluorophore Conjugation. In some embodiments, screening peptides or a library of peptides involves labeling of a protein of interest or a protein partner with a fluorophore or any other detectable moiety, such that detection of a signal from the fluorophore or detectable moiety is indicative of binding to the peptide. An example of a fluorophore that can be used is Alexa Fluor 647 NHS Ester (Life Technologies), which can be used to label a protein of interest as per manufacturer's protocol. Saturation is approximately 1 fluorophore per molecule, as determined by mass spectrometry. Free dye can be removed following labeling (e.g., by spin column dialysis).

Surface Plasmon Resonance (SPR) Interaction Analyses. In some embodiments, TfR-binding can be assessed using surface plasmon resonance (SPR). The peptides of the present disclosure can be tobacco etch virus (TEV)-cleavable and thus can be analyzed as independent, soluble proteins.

Various peptides of the present disclosure can be analyzed for binding affinity to TfR. Binding affinity can be analyzed by SPR experiments using captured biotinylated TfR, which can be performed at 25° C. on a Biacore T100 instrument (GE Healthcare) with Series S SA chips. HBS-EP+ (10 mM HEPES, pH 7.4, 150 mM NaCl, 3 mM EDTA, 0.05% surfactant P20) can be used as a running buffer in the experiments with 0.1 mg/mL bovine serum albumin (BSA). Soluble TfR-binding peptides can be evaluated for binding by incubation of a dilution series, in which the concentration range can be varied depending on the TfR-binding peptide being tested with 2 μg/ml TfR, capturing ˜300 resonance units (RUs) of protein for SPR experiments.

In some cases, SPR is performed by injecting the peptides over two densities of captured and biotinylated hTfR and the resulting data can then be analyzed globally. Binding constants (e.g., K_{D} values) and on- and off-rates can be calculated for the interactions between TfR and the TfR-binding peptide.

In some embodiments, SPR is performed with purified apo-transferrin and holo-transferrin. Holo-transferrin can be produced from apo-transferrin by loading apo-transferrin with an iron solution (e.g., 0.4 equivalents of Fe(NTA)₂ added every 5 minutes until 3.2 equivalents have been added). Excess iron can be subsequently removed through buffer exchange (e.g., spin column dialysis into PBS).

Protease Resistance Testing. Tumor environments, for instance, contain high concentrations of proteases. Furthermore, resistance to proteolysis (degradation or cleaving by proteases) reduces the likelihood of peptide degradation and subsequent immunogenicity, for example, in circulation. Resistance to proteolysis can further increase a peptide's serum half-life and the overall bioavailability of the peptide. Thus, it is advantageous for peptides of the present disclosure to be resistant to degradation by proteases.

To determine resistance to proteolytic digestion, various peptides of the present disclosure can be mixed with 50 U of porcine pepsin (Sigma-Aldrich P7012) in Simulated Gastric Fluid at pH 1.05, or 50 U of porcine trypsin (Sigma-Aldrich 6567) in PBS, incubated for 30 minutes at 37° C., and analyzed by reverse phase HPLC (RP-HPLC). Separately, the peptides can be mixed with 50 U of porcine pepsin (Sigma-Aldrich P7012) in Simulated Gastric Fluid43 at pH 1.05, or 50 U of porcine trypsin (Sigma-Aldrich 6567) in PBS, incubated for 30 m at 37° C., and analyzed by RP-HPLC. Control peptides can be incubated in PBS. All peptides can be incubated in non-reducing conditions. Alternatively, to determine stability in serum, the peptides or peptide oligonucleotide complexes can be mixed in serum and incubated for 30 m at 37° C. and analyzed by RP-HPLC.

In some embodiments, SDPR protease resistance testing can be performed having a sequence of SEQuencing-grade enzymes, including Trypsin and Chymotrypsin. Trypsin and trypsin inhibitor can be used for HPLC analysis.

Peptide Stability testing under Reducing Conditions. Peptides that can be designed or engineered to interact with one or more target proteins in the cell, such as proteins in the nucleus, can be exposed to reducing conditions in the cytosolic compartment of cells. Thus, it can be advantageous for peptides of this disclosure to display stability in reducing conditions. Stability of peptides of this disclosure can be tested in 10 mM dithiothreitol (DTT) and/or 10 mM glutathione (GSH). GSH can be a more physiologically relevant reducing agent for testing peptide stability in intracellular conditions. An example of a peptide-target protein interaction is a peptide binding to a TfR protein. Stability to the tested peptides can be evaluated using HPLC (or radio-HPLC when radiolabeled peptides are used), for example.

Thermal Shift Assay. Protein melting temperature (Tm) determination can be performed by monitoring protein unfolding using SYPRO Orange dye (Molecular Probes). In brief, 0.1 mg/mL protein sample in 20 μL total volume PBS buffer can be mixed with 2 μL of 10×SYPRO Orange dye. Dye intercalation into the hydrophobic protein core following protein unfolding was assayed using the C1000 Touch Thermal Cycler with CFX96 Deep Well Real-Time System (BioRad). Samples were heated from 20° C. to 95° C. with stepwise increments of 0.5° C. per minute and a 5 sec hold step for every point, followed by fluorescence reading. Tm were calculated by analyzing the derivatives of Relative florescence Units (RFU).

Cross Reactivity of TfR-binding Peptides to Murine TfR. Cross reactivity to human and murine Tf R of TfR-binding peptides as disclosed herein can be performed using cell surface binding assays. 293F cells expressing either human or mouse TfR from their surface were stained with soluble TfR-binding peptides that were directly labeled with AlexaFluor 647 dye. Flow cytometry can subsequently be used for data analysis to assess human versus mouse reactive if TfR-binding peptides.

Whole-body autoradiography to elucidate biodistribution of the TfR-binding peptides in mice. Whole body autoradiography (WBA) sagittal sectioning can be performed using the following steps. Each peptide can be radiolabeled by methylating lysines at the N-terminus. As such, the peptide can contain methyl or dimethyl lysines and a methylated or dimethlyated amino terminus. A dose of 100 nmol radiolabeled peptide can be administered via tail vein injection in Female Harlan athymic nude mice with intact kidneys, weighing 20-25 g. Each radiolabeled peptide can be allowed to freely circulate within the animal for the described time period before the animals are euthanized and sectioned.

All mice of treatment cohorts can be dosed with 100 nmol radiolabeled peptide oligonucleotide complex (˜20 mg/kg). Control mice can be injected with saline or PBS. At the end of the dosing period, mice are frozen in a hexane/dry ice bath and then embedded in a frozen block of carboxymethylcellulose. Whole animal sagittal slices can be prepared that resulted in thin frozen sections for imaging. Thin frozen sections can be obtained using a microtome and allowed visualization of tissues such as brain, tumor, liver, kidney, lung, heart, spleen, pancreas, muscle, adipose, gall bladder, upper gastrointestinal tract, lower gastrointestinal tract, bone, bone marrow, reproductive tract, eye, cartilage, stomach, skin, spinal cord, bladder, salivary gland, and more. Sections are allowed to desiccate in a freezer prior to imaging.

For the autoradiography imaging, tape mounted thin sections were freeze dried and radioactive samples can be exposed to phosphoimager plates for about 7 days. These plates are developed and the signal (densitometry) from each organ was normalized to the signal found in the cardiac blood of each animal. A signal in tissue darker than the signal expected from blood in that tissue indicates accumulation in a region, tissue, structure, or cell.

Reported quantitation of peptide in tissue (nmol/g) was accomplished by referencing an in-block standard curve and the peptide's specific ¹⁴C activity.

Serum and tissue quantitation of peptide levels over a time course. Depending on the utilized radionuclide either ex vivo liquid scintillation counting (e.g., ¹⁴C) or gamma counting (e.g., all positron emitting nuclides) can be used to determine peptide levels of various organs at specific time points as well as peptide behavior over time such as for blood half-life and organ uptake and clearance. All mice of treatment cohorts can be dosed with 100 nmol radiolabeled peptide oligonucleotide complex (˜20 mg/kg). Control mice can be injected with saline or PBS. Each radiolabeled peptidecan be allowed to freely circulate within the animal for the described time period before the animals were euthanized and sectioned. Collected organs including kidney, liver, brain, spleen, muscle, and skin can be homogenized or weighed before or after counting. Internal reference standards and control samples can also be counted. The peptide uptake data can be visualized as amount of peptide per gram tissue, or as percent injected dose per gram (or per mole or per volume).

General determination of peptide uptake in cells or tissues. The uptake of a peptide oligonucleotide complex as disclosed herein can be determined in a specific cell, cell population, tissue, or organ either ex vivo or in vivo. In the same way, the efficacy of cargo or payload delivery can be determined ex vivo or in vivo, for example, in cases where the cargo molecule comprises a detectable agent. Ex vivo analyses include organ harvest and fixation (e.g., using 4% formaldehyde) of harvested tissue prior to analyses. Tissue samples can be analyzed using a variety of analytical methods including microscopy, spectroscopy, flow cytometry, polymerase chain reaction (PCR), and via measurements of ultrasound, electromagnetic radiation (e.g., UV/VIS, X-ray) or radioactivity. For example, tissue uptake can be determined by measuring luminescence or bioluminescence of a cell, cell population, tissue, or organ sample, or by measuring radioactivity of a cell, cell population, tissue, or organ sample and by calculating uptake values such as percent injected dose per gram (or per mole or per volume). In addition, the acquisition of nuclear images visualizing the biodistribution at a specific point in time (e.g., static imaging) or over a time course (e.g., dynamic imaging) can be performed using various techniques (e.g., PET or SPECT) and appropriately radiolabeled peptides.

MTR BBB transcytosis model, and capillary depletion quantitation of parenchyma/capillary distribution. Mice (strain CD-1, N=1 per time point) can be anesthetized, and the left jugular vein and right carotid arteries can be surgically exposed. Labeled peptides (e.g., 0.25-1 μCi in about 200 μL volume; specific activities were 22-40 Ci/mol for SEQ ID NO: 65 and 127-150 Ci/mol for SEQ ID NO: 96) can be injected into the jugular vein. After a given period time within a time course (0-30 minutes), arterial blood can be collected, followed by decapitation and brain collection. Brains and serum can be tested via liquid scintillation counting for ¹⁴C levels, and the data can be incorporated into a multiple time regression mathematic model to quantify CNS accumulation rate. To determine whether this CNS accumulation is limited to capillaries or actually represents vascular transcytosis, a separate set of animals (N=3/peptide) can be euthanized 5 minutes after dosage, and brain homogenates are subjected to centrifugation through a dextran density gradient. This can allow separation of capillaries from parenchyma, and the two tissues can be subjected separately to scintillation counting to quantitate ¹⁴C signal. Normalized signal ratios can be an indicator of BBB penetration; for example, SEQ ID NO: 65 can have a substantially higher signal in the parenchyma than the capillaries, interpreted as evidence of BBB transcytosis.

### Methods for NT Fusions and Activation of the Neuronal CRE Reporter Mouse.

Transgenic mice expressing firefly luciferase under the control of a cyclic AMP response element (CRE) can be used. Under conditions of elevated cyclic AMP (cAMP) or other mechanisms that activate the transcription factor CRE Binding Protein 1 (CREB), cells in these mice can express luciferase, which can be measured in whole animal luminescence instrumentation (IVIS imager) after IV dosage with a luciferin formulated for animal use. Mice can be validated to express the transgene under proper response element control by dosing these mice with forskolin and rolipram, two drugs that stimulate neuronal cAMP production and inhibit its degradation (respectively). Animals dosed with forskolin and rolipram can then be tested for luciferase expression 4 hours later as described herein. High levels of luminescence from the animal's brain can be typical in these control experiments. Animals that validate in this assay can be given about 1 week to return to steady state (low) luciferase expression and can then be tested with neurotensin or TfR binding proteins fused with neurotensin. Neurotensin receptor expression can be limited to subsets of cells in the CNS, including the frontal cortex. Upon activation, a signal transduction pathway can be activated that culminates in CREB phosphorylation and CRE-mediated transcription. Its ligand, neurotensin, is a neuropeptide that cannot cross the BBB. Therefore, IV dosage of a fusion protein comprising neurotensin and a TfR binding peptide can demonstrate BBB penetration if CRE-driven luciferase is induced in the brains of these mice.

Peptide constructs, which are NT fusions include any sequence having at least 90%, at least 92%, at least 93%, at least 94%, at least 95%, at least 96%, at least 97%, at least 98%, at least 99%, or 100% sequence identity with any one of SEQ ID NO: 135-SEQ ID NO: 140.

### Methods of Manufacture

Nucleic acids, including RNA and DNA polynucleotides, used in the peptide nucleotide complexes described therein can also be produced using the methods described in U.S. Pat. No. 9,279,149, and is incorporated herein by reference. In some embodiments, RNA or DNA polynucleotides are synthesized by enzymatic/PCR methods. For example, RNA polynucleotides can be synthesized using an enzyme, such as a nucleotidyl transferase (e.g., E. coli poly(A) polymerase or E. coli poly(U) polymerase), which can add RNA nucleotides to the 3′ end. Alternatively, E. coli poly(U) polymerase can be used. A 3′ unblocked reversible terminator ribonucleotide triphosphates (rNTPs) can be used during polynucleotide synthesis. Alternatively, 3′blocked, 2′blocked, or 2′-3′ blocked rNTPs can be used alongside either enzyme described above. RNA or DNA polynucleotides can also be synthesized using standard solid-phase synthesis techniques and phosphoramidite-based methods or thiophosphorodiamidate methods. RNA or DNA polynucleotides of the present disclosure can be prepared by conventional solid phase oligonucleotide synthesis. For example any method of solid-phase synthesis can be employed including, but not limited to methods described, as shown at https://www.atdbio.com/content/17/Solid-phase-oligonucleotide-synthesis, and in Albericio (Solid-Phase Synthesis: A practical guide, CRC Press, 2000), Lambert et al. (Oligonucleotide Synthesis: Solid-Phase Synthesis, DNA, DNA Sequencing, RNA, Small Interfering RNA, Nucleoside, Nucleic Acid, Nucleotide, Phosphoramidite, Sense, Betascript Publishing, 2010), and Guzaev, A. P. et al. (Current Protocols in Nucleic Acid Chemistry. 2013; 53:3.1:3.1.1-3.1.60), each of which are incorporated herein by reference. Solid supports such as CPG or polystyrene can be used. Protected 2′-deoxynucleosides (dA, dC, dG, and T), ribonucleosides (A, C, G, and U), or chemically modified nucleosides, such as LNA or BNA can be used. Phosphoramidite chemistry can be used by cycling through the following steps: detritylation of the support-bound 3′-nucleoside, activation and coupling, capping, and oxidation. At the end of synthesis, the protected nucleotide can be cleaved from the support and then deprotected. The product can be purified by HPLC. Protecting groups used in solid-phase synthesis of RNA polynucleotides can include t-butyldimethylsilyl (TBDMS) or tri-iso-propylsilyloxymethyl (TOM). The RNA or DNA polynucleotides can have a modified backbone to enhance stability. Additionally, non-natural or modified bases can be used to serve as unique functional handles for subsequent chemical conjugation. In some embodiments, modification of the 5′ and or 3′ ends of the RNA or DNA can be performed to result in desired functional groups, stability, or activity. In some embodiments, the functional handles comprise modified bases including one or more modified uridine, modified guanosine, modified cytidine, or modified adenosine base of the RNA. An example of such modified base is a uridine with an extended amine. Nucleic acids, including a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter can be made using such methods. It may be advantageous to manufacture the oligonucleotide and the peptide by synthetic methods and then conjugate them together, with improved purity, safety, and cost of goods. Oligonucleotides, including modified oligonucleotides, may be manufactured by any of the methods disclosed in Glazier et al. Chemical synthesis and biological application of modified oligonucleotides. Bioconjugate Chemistry, 2020, 31, 1213-1233.

Various expression vector/host systems can be utilized for the recombinant expression of peptides described herein. Non-limiting examples of such systems include microorganisms such as bacteria transformed with recombinant bacteriophage DNA, plasmid DNA or cosmid DNA expression vectors containing a nucleic acid sequence encoding peptides, peptide constructs, or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV)), or transformed with recombinant plasmid expression vectors (e.g., Ti plasmid) containing the aforementioned nucleic acid sequence, or animal cell systems infected with recombinant virus expression vectors (e.g., adenovirus, vaccinia virus, lentivirus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secrete minimal amounts of proteolytic enzymes.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, peptides can also be synthesized in a cell-free system prior to extraction using a variety of known techniques employed in protein and peptide synthesis.

In some cases, a host cell produces a peptide that has an attachment point for a cargo molecule (e.g., a therapeutic agent). An attachment point could comprise a lysine residue, an N-terminus, a cysteine residue, a cysteine disulfide bond, a glutamic acid or aspartic acid residue, a C-terminus, or a non-natural amino acid. The peptide could also be produced synthetically, such as by solid-phase peptide synthesis, or solution-phase peptide synthesis. Peptide synthesis can be performed by fluorenylmethyloxycarbonyl (Fmoc) chemistry or by butyloxycarbonyl (Boc) chemistry. The peptide could be folded (formation of disulfide bonds) during synthesis or after synthesis or both. Peptide fragments could be produced synthetically or recombinantly. Peptide fragments can then be joined together enzymatically or synthetically.

In other aspects, the peptides of the present disclosure can be prepared by conventional solid phase chemical synthesis techniques, for example according to the Fmoc solid phase peptide synthesis method (“Fmoc solid phase peptide synthesis, a practical approach,” edited by W. C. Chan and P. D. White, Oxford University Press, 2000).

In some embodiments, the peptides of this disclosure can be more stable during manufacturing. For example, peptides of this disclosure can be more stable during recombinant expression and purification, resulting in lower rates of degradation by proteases that are present in the manufacturing process, a higher purity of peptide, a higher yield of peptide, or any combination thereof. In some embodiments, the peptides can also be more stable to degradation at high temperatures and low temperatures during manufacturing, storage, and distribution. For example, in some embodiments peptides of this disclosure can be stable at 25° C. In other embodiments, peptides of this disclosure can be stable at 70° C. or higher than 70° C. In some embodiments, peptides of this disclosure can be stable at 100° C. or higher than 100° C.

### Pharmaceutical Compositions

A pharmaceutical composition of the disclosure can be a combination of any peptide or peptide oligonucleotide complexes as described herein with other chemical components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients. The pharmaceutical composition facilitates administration of a peptide described herein to an organism. In some cases, the pharmaceutical composition comprises factors that extend half-life of the peptide or the nucleotide and/or help the peptide oligonucleotide complex to penetrate or be endocytosed by the target cells.

Pharmaceutical compositions can be administered in therapeutically-effective amounts as pharmaceutical compositions by various forms and routes including, for example, intravenous, subcutaneous, intramuscular, rectal, suppository, aerosol, parenteral, ophthalmic, pulmonary, transdermal, vaginal, optic, nasal, oral, sublingual, inhalation, dermal, intrathecal, intratumoral, intranasal, intravitreal, intravesical (such as by administration through a catheter to the bladder), and topical administration. A pharmaceutical composition can be administered in a local or systemic manner, for example, via injection of the peptide described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptide-antibody complexes described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such peptide-antibody complexes described herein to allow for the preparation of highly concentrated solutions.

### Formulation/Delivery

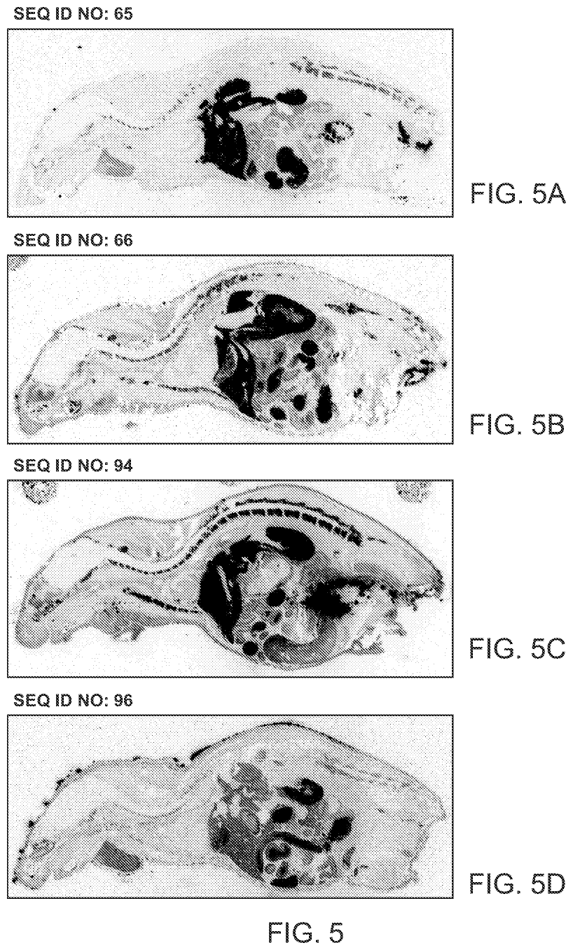

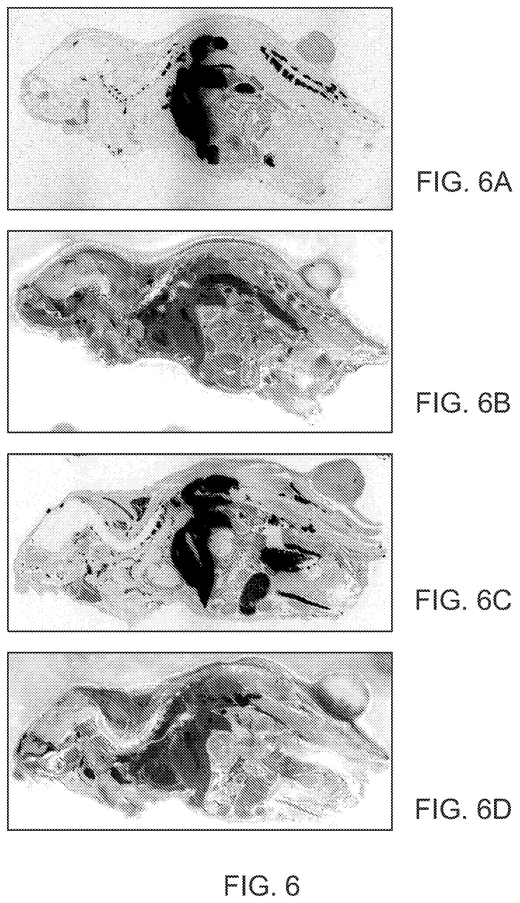

Peptides of the disclosure can be formulated to deliver a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter in various ways. The peptides can be conjugated, complexed, formulated or otherwise combined with oligonucleotides. The peptides and oligonucleotides or peptide-oligonucleotide conjugate (e.g., peptide oligonucleotide complex) can be formulated any of the ways disclosed in Hu Signal Transduction and Targeted Therapy 2020, 5:101 (hereby incorporated by reference, including FIGS. 5 and 6 therein). The peptides or peptide-oligonucleotide conjugates of this disclosure can be incorporated into lipid nanoparticles, decorated on lipid, peptidic, polymeric, or inorganic nanoparticles, incorporated in nanoplexes, liposomes, or exosomes, linked with polymers, or chemically conjugated to oligonucleotides.

The formulated peptide-oligonucleotide complex can be administered by intravenous, subcutaneous, intramuscular, intrathecal, intravitreal, or intratumoral injection. It can be delivered by electroporation into cells or microneedle delivery. It can be formulated as a liquid or as a solid or as a lyophilized solid.

On delivery to an endosome or any other cellular compartment, the oligonucleotide or any fragment of the peptide oligonucleotide complex may be slowly released to the cytosol or binding targets within the cell over the period of minutes, hours, days, weeks, or months. The delivery of the oligonucleotide or any fragment of the peptide oligonucleotide complex can optionally be further enhanced by adding an additional a cell penetrating peptide function to the formulation, complex, or conjugation. For clarity, a cell penetrating peptide functionality is not required for all embodiments as the TfR binding peptide itself is adequate for delivery of the peptide oligonucleotide complexes or any fragment of the peptide oligonucleotide complexes to tissues, cells, and subcellular compartments.

A peptide such as an albumin binding domain can be fused, conjugated, or combined with the peptide-oligonucleotide complex to extend the serum half-life or tissue exposure of the peptide-oligonucleotide. An albumin binding domain can include any of the proteins or sequences describe in Jacobs et al. Protein Engineering Design and selection, 2015, 1-9 which are incorporated herein by reference. An example of an albumin binding domain that can be included in any of the proteins, protein sequences, or protein complexes described herein is SEQ ID NO: 357.

Formulations or approaches to increase cell penetration or a peptide as describe herein can include, but not limited to, using high dosage of a peptide as described herein, such as up to 10 μM; conjugating or fusing a Tat peptide or an additional cell-penetrating peptide, or a variant or derivative thereof to a peptide; co-delivering a Tat peptide or an additional cell-penetrating peptide, or a variant or derivative thereof with a peptide; conjugating or fusing an Arg patch to a peptide; or co-delivering a peptide with an Arg patch peptide, such as up to 10 μM. In some embodiments, protein transfection agents, direct cytosolic expression of the peptide, or electrophoration of the peptide can be used to increase cell penetration. In some embodiments, other excipients can be formulated with a peptide in order to increase the cell penetration of the peptide, such as those approaches described in “Protein and Peptide Drug Delivery: Oral Approaches” Indian J. Pharm. Sci., Shaji and Patole, v70(3) 269-277, 2008. Any combination of these formulations or approaches can be used to increase cell penetration of a peptide as described herein.

Alternatively, the peptide described herein can be lyophilized or in powder form for re-constitution with a suitable vehicle, e.g., sterile pyrogen-free water, buffer, saline, or sugar solution, before use. In some embodiments, a purified peptide is administered intravenously. A peptide described herein can be administered to a subject in order to home, target, migrate to, or be directed to a CNS cell, a brain cell, a cancerous cell, or a tumor. In some embodiments, a peptide can be conjugated to, linked to, or fused to another peptide that provides a targeting function to a specific target cell type in the central nervous system or across the blood brain barrier. Exemplary target cells include a CNS cell, erythrocyte, an erythrocyte precursor cell, an immune cell, a stem cell, a muscle cell, a brain cell, a thyroid cell, a parathyroid cell, an adrenal gland cell, a bone marrow cell, an appendix cell, a lymph node cell, a tonsil cell, a spleen cell, a muscle cell, a liver cell, a gallbladder cell, a pancreas cell, a cell of the gastrointestinal tract, a glandular cell, a kidney cell, a urinary bladder cell, an endothelial cell, an epithelial cell, a choroid plexus epithelial cell, a neuron, a glial cell, an astrocyte, or a cell associated with a nervous system.

A peptide of the disclosure can be applied directly to an organ, or an organ tissue or cells, such as the CNS, brain or brain tissue or cells, or eye or eye cells during a surgical procedure. The recombinant peptide described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically effective amounts of a peptide oligonucleotide complex described herein can be administered in pharmaceutical compositions to a subject suffering from a condition that affects the immune system. In some embodiments, the subject is a mammal such as a human or a primate. A therapeutically effective amount can vary widely depending on the severity of the disease, the age and relative health of the subject, the potency of the compounds used, and other factors.

In some embodiments, a peptide is cloned into a viral or non-viral expression vector. Such expression vector can be packaged in a viral particle, a virion, or a non-viral carrier or delivery mechanism, which is administered to patients in the form of gene therapy. In other embodiments, patient cells are extracted and modified to express a peptide capable of binding TfR ex vivo before the modified cells are returned back to the patient in the form of a cell-based therapy, such that the modified cells will express the peptide once transplanted back in the patient.

Pharmaceutical compositions can be formulated using one or more physiologically acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically acceptable salt form.

Methods for the preparation of peptide described herein comprising the compounds described herein include formulating peptide described herein with one or more inert, pharmaceutically acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Pharmaceutical compositions can also include permeation or absorption enhancers (Aungst et al. AAPS J. 14(1):10-8. (2012) and Moroz et al. Adv Drug Deliv Rev101:108-21. (2016)). Permeation enhancers can facilitate uptake of molecules from the GI tract into systemic circulation. Permeation enhancers can include salts of medium chain fatty acids, sodium caprate, sodium caprylate, N-(8-[2-hydroxybenzoyl]amino)caprylic acid (SNAC), N-(5-chlorosalicyloyl)-8-aminocaprylic acid (5-CNAC), hydrophilic aromatic alcohols such as phenoxyethanol, benzyl alcohol, and phenyl alcohol, chitosan, alkyl glycosides, dodecyl-2-N,N-dimethylamino propionate (DDAIPP), chelators of divalent cations including EDTA, EGTA, and citric acid, sodium alkyl sulfate, sodium salicylate, lecithin-based, or bile salt-derived agents such as deoxycholates.

Compositions can also include protease inhibitors including soy bean trypsin inhibitor, aprotinin, sodium glycocholate, camostat mesilate, vacitracin, or cyclopentadecalactone.

### Use of Peptide Oligonucleotide Complexes in Treatments

In some embodiments, the method includes administering an effective amount of a peptide oligonucleotide complex as described herein to a subject in need thereof. In one embodiment, the method includes administering an effective amount of a peptide oligonucleotide as described herein to a subject in need thereof.

TfR can be expressed in various tissues such as the brain, the stomach, the liver, of the gall bladder. Hence, the TfR-binding peptides and peptide oligonucleotide complexes of the present disclosure can be used in the diagnosis and treatment of disease and conditions associated with various tissues and organs. For example, drug delivery to these tissues and organs can be improved by using the herein described peptides, peptide constructs, and peptide complexes carrying a diagnostic and/or therapeutic payload. In some embodiments, the therapeutic payload may comprise a nucleotide targeting agent, an additional therapeutic agent, a detectable agent, or combinations thereof.

A peptide oligonucleotide complex may reduce a phenotype or symptom associated with a disease upon administration to a subject. In some embodiments, a phenotype is reduced by at least 10%, 25%, 30%, 40%, 50%, 60%, 75%, 80%, 90%, 95%, 99%, 99.5%, or 99.9%. In some embodiments, a symptom is reduced by at least 10%, 25%, 30%, 40%, 50%, 60%, 75%, 80%, 90%, 95%, 99%, 99.5%, or 99.9%.

A peptide oligonucleotide complex may be stable upon administration to a subject. In some embodiments, at least 50% of the peptide oligonucleotide complex remains intact up to 5 min, 15 min, 30 min, 45 min, 1 hour, 2 hours, 4 hours, 8 hours, 12 hours, or 24 hours after administering to a subject. In some embodiments, at least 50% of the peptide oligonucleotide complex remains intact up to 5 min, 15 min, 30 min, 45 min, 1 hour, 2 hours, 4 hours, 8 hours, 12 hours, or 24 hours after contacting to human serum.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case can be determined using techniques, such as a dose escalation study.

The methods, compositions, and kits of this disclosure can comprise a method to prevent, treat, arrest, reverse, or ameliorate the symptoms of a condition. The treatment can comprise treating a subject (e.g., an individual, a domestic animal, a wild animal, or a lab animal afflicted with a disease or condition) with a peptide of the disclosure. The disease can be a cancer or tumor. In treating the disease, the peptide can contact the tumor or cancerous cells. The subject can be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment can be provided to the subject for more than 1 day, 1 week, 1 month, 6 months, 12 months, 2 years or more after clinical onset of disease. Treatment can be provided to the subject for less than 1 day, 1 week, 1 month, 6 months, 12 months, or 2 years after clinical onset of the disease. Treatment can also include treating a human in a clinical trial. A treatment can comprise administering to a subject a pharmaceutical composition, such as one or more of the pharmaceutical compositions described throughout the disclosure. A treatment can comprise a once daily dosing. A treatment can comprise delivering a peptide of the disclosure to a subject, either intravenously, subcutaneously, intramuscularly, by inhalation, dermally, topically, by intra-articular injection, orally, sublingually, intrathecally, transdermally, intranasally, via a peritoneal route, directly into a tumor e.g., injection directly into a tumor, directly into the brain, e.g., via and intracerebral ventricle route, or directly onto a joint, e.g. via topical, intra-articular injection route. A treatment can comprise administering a peptide-active agent complex to a subject, either intravenously, subcutaneously, intramuscularly, by inhalation, by intra-articular injection, dermally, topically, orally, intrathecally, transdermally, intranasally, parenterally, orally, via a peritoneal route, nasally, sublingually, or directly onto cancerous tissues.

Binding of the herein described peptides, peptide constructs, and peptide complexes (e.g., peptide conjugates, complexes, or recombinantly produced peptide constructs) to TfR and subsequent transport across a cell layer or barrier such as the BBB (e.g., via vesicular transcytosis) or a cell membrane (e.g., via endocytosis such as receptor-mediated endocytosis) or accumulation in a tissue can have implications in a number of diseases, conditions, or disorders such as those associated with cell growth, cell proliferation, angiogenesis, organogenesis, tumor progression, and/or metastasis, cell death (e.g., senescence), neurodegeneration, and pain in various cells, tissues and organs, particularly those cells tissues or organs that express and/or overexpress TfR. In some embodiments, peptide constructs (e.g., peptide-NT constructs) interact with dopamine-signaling neurons to reduce pain. Compositions comprising any one of the peptides disclosed herein that bind TfR, or a pharmaceutical composition thereof, can be used in a method of treating, preventing, or diagnosing a cancer, tumor progression, dysregulated cell growth in a variety of cancers including ovarian cancer, colon cancer, glioblastoma, astrocytoma, glioma, medulloblastoma, ependymoma, choroid plexus carcinoma, midline glioma, diffuse intrinsic pontine glioma, and lung cancer, cancer located in the bone or bone marrow, glioblastoma, astrocytoma, glioma, medulloblastoma, ependymoma, choroid plexus carcinoma, midline glioma, and diffuse intrinsic pontine glioma (DIPG), breast cancer, liver cancer, colon cancer, brain cancer, spleen cancer, cancers of the salivary gland, kidney cancer, muscle cancers, bone marrow cell cancers, or skin cancer, genitourinary cancer, osteosarcoma, muscle-derived sarcoma, melanoma, head and neck cancer, a neuroblastoma, or a CMYC-overexpressing cancer. In some embodiments, the TfR-binding peptides of the present disclosure enable delivery to tumor cells, bone marrow cells (e.g., erythroid precursor cells), spleen cells, immune cells, cancers of the salivary gland, or muscle cells. The TfR-binding peptides of the present disclosure can be used in combination with drug used for the treatment or prevention of neurological and CNS-related disorder (e.g., neurodegenerative diseases). Additional diseases, disorders, or conditions can include multiple myeloma, plastic anemia, myelodysplasia, and related bone marrow failure syndromes, myeloproliferative diseases, acute and chronic myeloid leukemia, malignancies of lymphoid cells, hematologic malignancies, plasma cell disorders, skeletal muscle disorder, myopathy, muscular dystrophy (e.g., Becker muscular dystrophy, Duchenne muscular dystrophy, Emery-Dreifuss muscular dystrophy, Facioscapulohumoeral muscular dystrophy, Myotonia congentia, and myotonic dystrophy), and chronic obstructive pulmonary disorder. In some embodiments, compositions/peptides disclosed herein are used to treat dysregulated cell growth, cancer, tumor, and/or metastasis associated with any of the following cell, tissue, or organ types: brain, lung, liver, spleen, kidney, lymph node, small intestine, blood cell, bone marrow cell, hematopoietic stem cell, pancreatic, colon, stomach, cervix, breast, endometrial, muscular, connective, prostate, testicle, ovarian, skin, head and neck, esophageal, oral tissue, and bone marrow. In further embodiments, compositions/peptides disclosed herein are used to treat nay of the following: sarcoma, osteosarcoma, muscle-derived sarcoma, hepatocellular carcinoma, malignant mesothelioma, schwannoma, meningioma, renal carcinoma, cholangiocarcinoma, bile duct hamartoma, soft tissue carcinoma, myeloma, multiple myeloma, leukemia, lymphoma, ovarian carcinoma, colonic adenoma, T cell acute lymphoblastic leukemia, gastrointestinal hyperplasia, fibrosarcoma, pancreatic ductal metaplasia, squamous cell carcinoma, kaposis sarcoma, and HIV-induced non-Hodgkin's lymphoma.

In various embodiments, the present disclosure provides methods and compositions that enable TfR-mediated transport across cellular layers (e.g., endothelial cells or epithelial cells) or cell membranes. In addition to the BBB, various other cells, tissues, and organs express TfR. Single cells expressing TfR can include hepatocytes, erythrocytes and erythrocyte precursors in bone marrow, immune cells, stem cells, and rapidly dividing cells. Tissues and organs expressing TfR can include the brain (e.g., cerebral cortex, hippocampus, caudate, cerebellum), endocrine tissues (e.g., thyroid, parathyroid, and adrenal glands), bone marrow and immune system (e.g., appendix, lymph node, tonsil, spleen), muscle tissues (e.g., heart, skeletal, and smooth muscle), liver, gallbladder, pancreas, gastrointestinal tract (e.g., oral mucosa, esophagus, stomach, duodenum, small intestine, colon, rectum), kidney, urinary bladder, female tissues (e.g., fallopian tube, breast, vagina, cervix, endometrium, ovary, and placenta), adipose and soft tissue, and skin. Thus, the TfR-binding peptides of the present disclosure can be used to target these cells, tissues, and organs and deliver an active agent to these cells, tissues, and organs via, for example, TfR-mediated transcytosis (e.g., across cellular barrier such as the BBB) or TfR-mediated endocytosis (e.g., across cell membranes into cells).

In various embodiments, the present disclosure provides methods and compositions that enable TfR-mediated transport and delivery to tumor cells expressing TfR. Cancers overexpressing TfR can include ovarian cancer, colon cancer, lung cancer, cancer located in the bone or bone marrow, glioblastoma, astrocytoma, glioma, medulloblastoma, ependymoma, choroid plexus carcinoma, midline glioma, diffuse intrinsic pontine glioma (DIPG), breast cancer, liver cancer, colon cancer, brain cancer, spleen cancer, cancers of the salivary gland, kidney cancer, muscle cancers, bone marrow cell cancers, skin cancer, genitourinary cancer, osteosarcoma, muscle-derived sarcoma, melanoma, head and neck cancer, neuroblastoma, prostate cancer, bladder cancer, acute lymphocytic leukemia, acute myeloid leukemia, chronic lymphocytic leukemia, chronic myeloid leukemia, Hodgkin lymphoma, Non-Hodgkin lymphoma, or a CMYC-overexpressing cancer. Brain cancers include but are not limited to glioblastoma, astrocytoma, glioma, medulloblastoma, ependymoma, choroid plexus carcinoma, midline glioma, and diffuse intrinsic pontine glioma.

TfR-binding peptide oligonucleotide complexes that can be used to prevent and/or treat a cancer are those comprising a TfR binding peptide and an active agent with anti-tumor activity such as an IL15, a fusion of IL15/IL15Ra, IFNgamma, and anti-CD3 agents. An active agent with active tumor activity (e.g., IL15, a fusion of IL15/IL15Ra, IFNgamma, or anti-CD3 agents) may be fused to a TfR-binding peptide (e.g., any one of SEQ ID NO: 1-SEQ ID NO: 134 or SEQ ID NO: 306-SEQ ID NO: 335) to form a peptide-active agent construct.

Binding of the herein described peptides and peptide constructs and peptide complexes (e.g., peptide conjugates or recombinantly produced peptide constructs) to TfR and subsequent transport across a cell layer or barrier such as the BBB (e.g., via TfR-mediated vesicular transcytosis) or a cell membrane (e.g., via TfR-mediated endocytosis) can have implications in a number of diseases, conditions, or disorders associated with chronic pain (e.g., headaches or migraine), neuropathic pain, obesity, insulin resistance, opioid addiction, or other neurologic or psychiatric disorders in a subject (e.g., a human). In some embodiments, peptide constructs (e.g., peptide-NT constructs) interact with dopamine-signaling neurons to reduce pain.

Binding of the herein described peptides, peptide constructs, and peptide complexes (e.g., peptide conjugates, complexes, fusion peptides, or recombinantly produced peptide constructs) to TfR and subsequent transport across a cell layer or barrier such as the BBB (e.g., via vesicular transcytosis) or a cell membrane (e.g., via endocytosis) can have implications in a number of diseases, conditions, or disorders associated with neurodegeneration. Neurodegenerative diseases that can treated, prevented, or diagnosed with the herein described TfR-binding peptides can include Alzheimer's disease, Amyotrophic lateral sclerosis, Friedreich's ataxia, Huntington's disease, Lewy body disease, Parkinson's disease, Spinal muscular atrophy, Motor neuron disease, Lyme disease, Ataxia-telangiectasia, Autosomal dominant cerebellar ataxia, Batten disease, Corticobasal syndrome, Creutzfeldt-Jakob disease, Fragile X-associated tremor/ataxia syndrome, Kufor-Rakeb syndrome, Machado-Joseph disease, multiple sclerosis, chronic traumatic encephalopathy, or frontotemporal dementia. In some cases, the TfR-binding peptide can be used in combination with BACE inhibitors, galantamine, amantadine, benztropine, biperiden, bromocriptin, carbidopa, donepezil, entacapone, levodopa, pergolie, pramipexole, procyclidine, rivastigmine, ropinirole, selegiline, tacrine, tolcapone, or trihexyphenidyl to treat and/or prevent a neurodegenerative disease.

In some embodiments, modulation (e.g., inhibition) of ion channels such as Kᵥ1.3 potassium channels using TfR-binding peptides and ion channel modulators such as Kᵥ1.3 potassium channel inhibitor conjugates oligonucleotide complexes can be used for treating or preventing inflammation in the brain. Kᵥ1.3 potassium channels, for instance, can be highly expressed on microglia in the brain. Thus, neuroinflammatory and neurodegenerative diseases such as multiple sclerosis, Alzheimer's, Parkinson's, traumatic brain injury, radiation therapy toxicity and other neurodegenerative and neuroinflammatory diseases can be treated with TfR-binding peptides that are conjugated to, linked to, or fused to a Kᵥ1.3 inhibitor. These diseases can be marked by upregulation of Kᵥ1.3. In some cases, Kᵥ1.3 inhibition can be used for treatment of psoriasis and other non-brain autoimmune diseases due to its effect on effector T cells.

In some embodiments, the TfR-binding peptides of the present disclosure can be used for the treatment and prevention of Crohn's disease or, more generally, inflammatory bowel diseases. In some embodiments, the TfR-binding peptides of the present disclosure show high uptake and retention in glandular cells of the intestine, which can express high amounts of TfR.

In some embodiments, the peptide oligonucleotide complexes of the present disclosure (e.g., comprising a peptide of SEQ ID NO: 1-SEQ ID NO: 134 or SEQ ID NO: 306-SEQ ID NO: 335) are used to access and treat these disorders due to their enhanced stability in various biological environments, including low pH, protease-rich environments, acidic environments, reducing environments, or environments with varying temperatures.

In some embodiments, compositions comprise any one of the TfR-binding peptides disclosed herein and are capable of transporting one or more cargo molecules or active agents per TfR-binding peptides across cell layers or cell barriers such as endothelial layers (e.g., the BBB) or epithelial layers, or cell membranes. In some embodiments, the cargo molecule or active agent is an immunotherapeutic agent, a CTLA-4 targeting agent, a PD-1 targeting agent, a PDL-1 targeting agent, an IL15, a fusion of IL-15/IL-15Ra complex agent, an IFNgamma agent, an anti-CD3 agent, an ion channel modulator, a Kᵥ1.3 inhibitor, an auristatin, MMAE, a maytansinoid, DM1, DM4, doxorubicin, a calicheamicin, a platinum compound, cisplatin, a taxane, paclitaxel, SN-38, a BACE inhibitor, a Bcl-xL inhibitor, WEHI-539, venetoclax, ABT-199, navitoclax, AT-101, obatoclax, a pyrrolobenzodiazepine or pyrrolobenzodiazepine dimer, a dolastatin, or a neurotransmitter such as neurotensin.

### Peptide Kits

In one aspect, peptides described herein can be provided as a kit. In another embodiment, peptide constructs described herein can be provided as a kit. In another embodiment, a kit comprises amino acids encoding a peptide described herein, a vector, a host organism, and an instruction manual. In some embodiments, a kit includes written instructions on the use or administration of the peptides.

While certain embodiments of the present disclosure have been exemplified or shown and described herein, it will be apparent to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all embodiments of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

### EXAMPLES

The following examples are included to further describe some aspects of the present disclosure and should not be used to limit the scope of the invention.

### Example 1

### Synthesis of a Peptide Oligonucleotide Complex for Antisense Therapy

A gene targeted for silencing in order to address a disease is identified and the desired single-stranded antisense oligonucleotide sequence is designed and synthesized based on the target coding or complementary sequence. The antisense oligonucleotide is conjugated to any TfR binding peptide disclosed herein, including peptides of any one of SEQ ID NO: 1-SEQ ID NO: 134 or SEQ ID NO: 306-SEQ ID NO: 335 by any of the methods disclosed herein, for example, in accordance with EXAMPLE 8-EXAMPLE 13, such as with a cleavable or stable linker. Optionally, a nucleotide (including the backbone) is modified, such as to increase in vivo stability, to increase resistance to enzymes such as nucleases, increase protein binding including to serum proteins, increase in vivo half-life, to modify the tissue biodistribution, or to reduce immune system activation.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 2

### Synthesis of a Peptide-Oligonucleotide Conjugate for RNAi Therapy

A gene targeted for silencing in order to address a disease is identified and the desired double-stranded RNAi sequence is designed and synthesized based on the target coding or complementary sequence. The sense or the antisense oligonucleotide of the RNAi is conjugated to any TfR binding peptide disclosed herein, including a peptide of any one of SEQ ID NO: 1-SEQ ID NO: 134 or SEQ ID NO: 306-SEQ ID NO: 335 by any of the methods disclosed herein, for example, in accordance with EXAMPLE 8-EXAMPLE 13, such as with a cleavable or stable linker. Optionally the peptide is conjugate to the sense (passenger) strand of the oligonucleotide. Optionally, a nucleotide (including the backbone) is modified, such as to increase in vivo stability, to increase resistance to enzymes such as nucleases, increase protein binding including to serum proteins, increase in vivo half-life, to modify the tissue biodistribution, or to reduce immune system activation. The sense and antisense strands are hybridized together, either before or after the conjugation.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 3

### Synthesis of a Peptide-Oligonucleotide Conjugate for U1 Adaptor Therapy

A gene targeted for silencing in order to address a disease is identified and the desired oligonucleotide sequence for U1 adaptor therapy is designed and synthesized based on the target coding or complementary sequence. The oligonucleotide is conjugated to any to any TfR binding peptide disclosed herein, including peptide of SEQ ID NO: 1-SEQ ID NO: 134 or SEQ ID NO: 306-SEQ ID NO: 335 by any of the methods disclosed herein, for example, in accordance with EXAMPLE 8-EXAMPLE 13, such as with a cleavable or stable linker. Optionally, a nucleotide (including the backbone) is modified, such as to increase in vivo stability, to increase resistance to enzymes such as nucleases, increase protein binding including to serum proteins, increase in vivo half-life, to modify the tissue biodistribution, or to reduce immune system activation.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 4

### Synthesis of a Peptide-Oligonucleotide Conjugate for Aptamer Therapy

An aptamer sequence that interacts with a target molecule is selected to address a disease is identified against the target and synthesized. The aptamer oligonucleotide is conjugated to any TfR binding peptide disclosed herein, including any peptide of SEQ ID NO: 1-SEQ ID NO: 134 or SEQ ID NO: 306-SEQ ID NO: 335 by any of the methods disclosed herein, for example, in accordance with EXAMPLE 8-EXAMPLE 13, such as with a cleavable or stable linker. Optionally, a nucleotide (including the backbone) is modified, such as to increase in vivo stability, to increase resistance to enzymes such as nucleases, increase protein binding including to serum proteins, increase in vivo half-life, to modify the tissue biodistribution, or to reduce immune system activation.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 5

### Conjugation of an Oligonucleotide and a Peptide Using Click Chemistry

An alkyne or azide group is installed in an oligonucleotide, such as by adding a hexynyl group to the 5′ end or the 3′ end of the oligonucleotide, installation of a 5-Octadiynyl dU, installation of a DIBO at the 5′ end using, which is optionally installed using a DIBO phosphoramidite, or installation of an azide group by use of an NHS ester reaction linking an azide group to a dT base. An azide or an alkyne group is installed on a peptide, such as by incorporating an N-terminal 6-azidohexanoic acid, an azidohomoalanine residue, or homopropargyl glycine residue. Optionally, the alkyne group comprises a strained ring such as strained cyclooctyne ring, such as DIBO. The oligonucleotide is conjugated to any TfR binding peptide disclosed herein, including any peptide of SEQ ID NO: 1-SEQ ID NO: 134 or SEQ ID NO: 306-SEQ ID NO: 335. The oligonucleotide and the peptide are conjugated together by combining an azide group on one with the alkyne group on the other using a copper-catalyzed azide-alkyne cycloaddition or a strain-promoted azide-alkyne cycloaddition to form a triazole bond.

Any peptide oligonucleotide complexes of the present disclosure described in EXAMPLE 1-EXAMPLE 7 alkyne or azide group may be so modified and are described. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 6

### Conjugation of an RNAi Sequence and a Peptide Using Click Chemistry

An alkyne group within a strained cyclooctyne is installed on an oligonucleotide, optionally linked to either the 5′ or the 3′ end of a sense or antisense strand. Optionally the strained cyclooctyne is DIBO, which is optionally installed on the 5′ end using a DIBO phosphoramidite. An azide group is installed on a peptide. The peptide is optionally the sequence given in TABLE 12. Optionally, the peptide is prepared as a TFA salt form. The alkyne-containing oligonucleotide and the azide-containing peptide are contacted together, such as in a buffer, solution, or solvent. The azide and the alkyne react to form a triazole bond that links the oligonucleotide and the peptide. The sense and antisense strands of the RNAi are hybridized together, either before or after the conjugation reaction.

TABLE 12
Examples of Azide-Containing Peptides

| SEQ ID NO | Sequence |
|---|---|
| SEQ ID NO: 129 | XREGCASRCMKYNDELEKCEARMMSMSNTEEDCEQELEDLLYC |
| | LDHCHSQ |
| SEQ ID NO: 130 | XREGCASRCTKYNAELEKCEARVMSMSNTEEDCEQELEDLLHCL |
| | DHCHSQ |
| SEQ ID NO: 131 | XGVZGREGCASRCMKYNDELEKCEARMMSMSNTEEDCEQELED |
| | LLYCLDHCHSQ |
| SEQ ID NO: 132 | XGREGCASRCMKYNDELEKCEARMMSMSNTEEDCEQELEDLLY |
| | CLDHCHSQ |
| SEQ ID NO: 133 | XGREGCASRCTKYNAELEKCEARVMSMSNTEEDCEQELEDLLH |
| | CLDHCHSQ |
| SEQ ID NO: 134 | XGVZGGREGCASRCMKYNDELEKCEARMMSMSNTEEDCEQELE |
| | DLLYCLDHCHSQ |

X = 6-azidohexanoyl
Z = citrulline

Any peptide oligonucleotide complexes of the present disclosure described in EXAMPLE 1-EXAMPLE 7 alkyne group within a strained cyclooctyne may be so installed and are described. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 7

### Conjugation of a U1 Adapter Sequence and a Peptide Using Click Chemistry

An alkyne group within a strained cyclooctyne is installed on an oligonucleotide, optionally linked to either the 5′ or the 3′ end of a sequence, designed for U1 adapter therapy. Optionally the strained cyclooctyne is DIBO, which is optionally installed on the 5′ end using a DIBO phosphoramidite. An azide group is installed on a peptide. The peptide is optionally the sequence provided in TABLE 12 of EXAMPLE 5. Optionally, the peptide is prepared as a TFA salt form. The alkyne-containing oligonucleotide and the azide-containing peptide are contacted together, such as in a buffer, solution, or solvent. The azide and the alkyne react to form a triazole bond that links the oligonucleotide and the peptide.

Any peptide oligonucleotide complexes of the present disclosure described in EXAMPLE 1-EXAMPLE 7 alkyne group within a strained cyclooctyne may be so installed and are described. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 8

### Installation of a Thiol Group, an Amine Group, or an Aldehyde Group in an Oligonucleotide

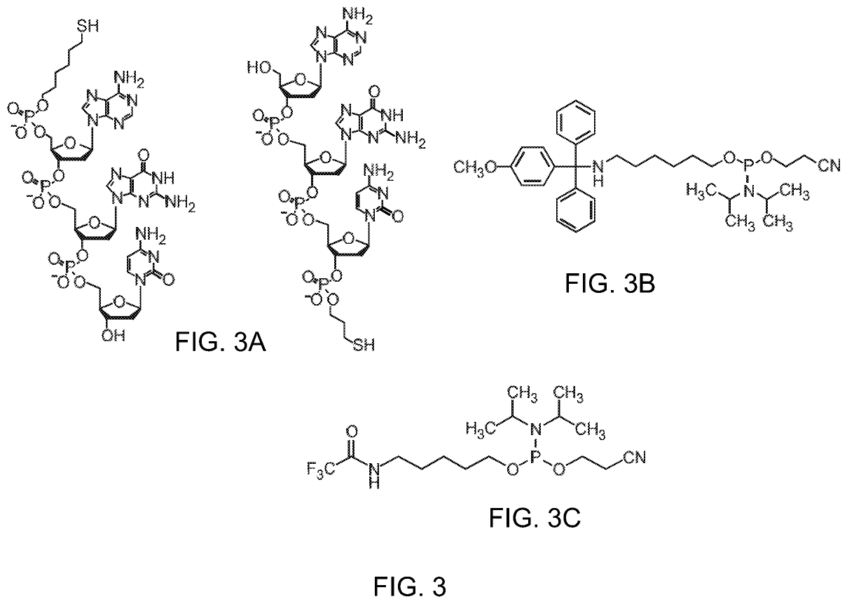

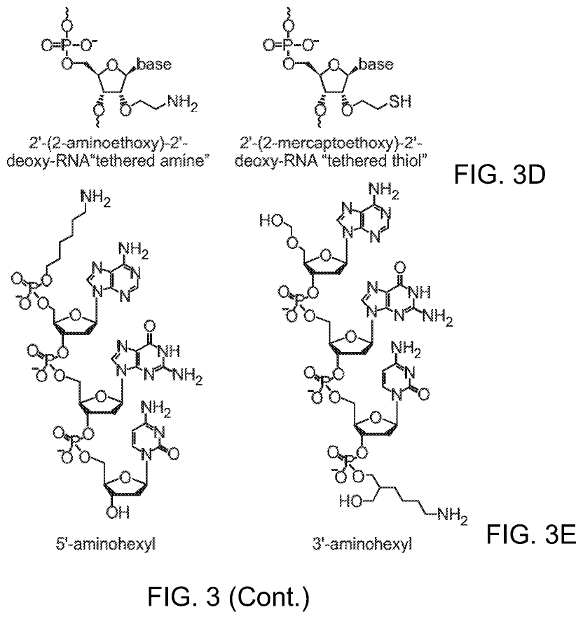

This example describes incorporation of a thiol group, an amine group, or an aldehyde group in RNA or DNA or any oligonucleotide. FIG. 3 illustrates incorporation or addition of these groups on RNA or DNA. A thiol group is added on an oligonucleotide, using EDC and imidazole to activate the 5′ phosphate group to a phosphorylimidazolide, and by subsequently reacting the resulting product with cystamine. This is followed by reduction with dithiothreitol (DTT) to form a phosphoramidite linkage to a free thiol group. A thiol group is, alternatively, added on an oligonucleotide by incorporating a phosphoramidite that contains a thiol during solid-phase phosphoramidite oligonucleotide synthesis, at either the 5′-end or the 3′-end of the oligonucleotide as shown in FIG. 3. The phosphoramidite used during synthesis can have a protecting group on the thiol during synthesis that is removed during cleavage, purification, and workup. FIG. 3A illustrates structures of oligonucleotides containing a 5′-thiol (thiohexyl; C6) modification (left), and a 3′-thiol (C3) modification (right), as shown at https://www.atdbio.com/content/50/Thiol-modified-oligonucleotides.

An amine group is added on RNA or DNA by incorporating a phosphoramidite during synthesis that contains a protected amino group that is later deprotected. FIG. 3B illustrates an MMT-hexylaminolinker phosphoramidite. FIG. 3C illustrates a TFA-pentylaminolinker phosphoramidite, as shown at https://www.sigmaaldrich.com/catalog/product/sigma/m01023hh?lang=en&region=US.

Alternatively, thiol or amine containing oligonucleotide residues are included within the sequence at any chosen location in RNA or DNA, such as described by Jin et al. (J Org Chem. 2005 May 27; 70(11):4284-99). FIG. 3D illustrates RNA residues incorporating amine or thiol residues, as presented in Jin et al. (J Org Chem. 2005 May 27; 70(11):4284-99). Also, an oligonucleotide residue that contains a phosphorothioate group within the phosphodiester backbone (where a sulfur atom replaces a non-bridging oxygen in the phosphate backbone of the oligonucleotide) provides a reactive group that is similarly used for conjugation to a thiol group. Use of the phosphorothioate containing residues can also make the RNA more resistant to nuclease degradation.

FIG. 3E illustrates oligonucleotides with aminohexyl modifications at the 5′ (left) and 3′ ends (right).

Aldehyde functional groups can be incorporated at the 3′ end of RNA by using periodate oxidation to convert the diol into two aldehyde groups.

Other methods of incorporating or modifying functional groups are carried out using techniques set forth in Bioconjugate Techniques, by Greg Hermanson, 3rd edition.

Any peptide oligonucleotide complexes of the present disclosure described in EXAMPLE 1-EXAMPLE 7 thiol group, an amine group, or an aldehyde group may be so installed and are described. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 9

Generation of Cleavable Linkers Between an Oligonucleotide with a Peptide

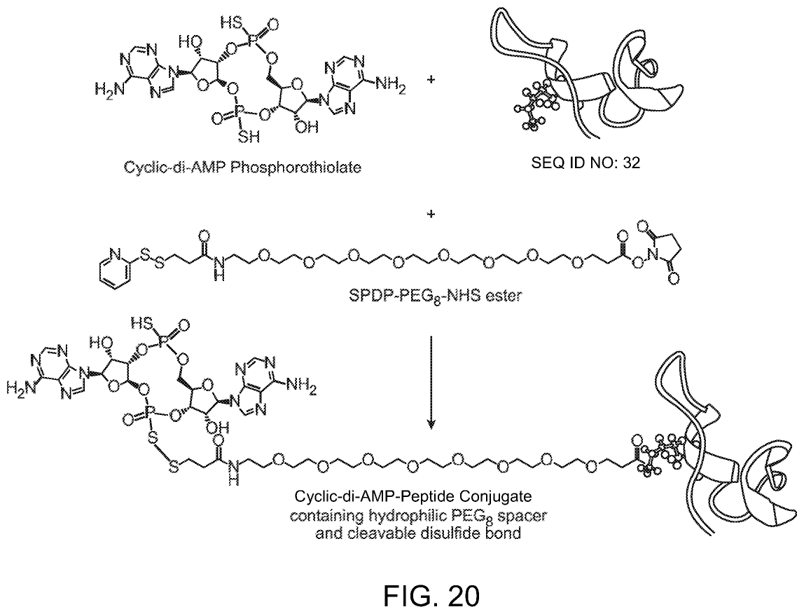

This example describes generation of cleavable linkers between an oligonucleotide with any one of peptides of SEQ ID NO: 1-SEQ ID NO: 134 or SEQ ID NO: 306-SEQ ID NO: 335. A disulfide linker is generated by combining a thiol-containing oligonucleotide with a peptide comprising a free thiol group. The thiol is incorporated on the peptide using Traut's reagent, SATA, SPDP or other appropriate reagents on a reactive amine (such as a heterobifunctional SPDP and NHS ester linker with the N-terminus or a lysine residue), or by incorporating a free cysteine residue in the peptide, as shown in FIG. 20. The disulfide linker is cleaved in the reducing environment of the cytoplasm or in the endosomal/lysosomal pathway.

An ester linkage is generated by combining a free hydroxyl group (such as on the 3′ end of an oligonucleotide) with a carboxylic acid group on the peptide (such as from the C-terminus, an aspartic acid, glutamic acid residue, or introduced via a linker to a lysine residue or the N-terminus) such as via Fisher esterification or via use of an acyl chloride. The ester linker is cleaved by hydrolysis, which is accelerated by the lower pH of endosomes and lysosomes, or by enzymatic esterase cleavage.

An oxime or hydrazone linkage is generated by combining an aldehyde group on the oligonucleotide with a peptide that has been functionalized with an aminooxy group (to form an oxime linkage) or a hydrazide group (to form a hydrazone linkage). The stability or lability of an oxime or hydrazone linkage is tailored by neighboring groups (Kalia et al., Angew Chem Int Ed Engl. 2008; 47(39):7523-6), and hydrolytic cleavage is accelerated in acidic compartments such as the endosome/lysosome.

A hydrazide group is incorporated on a peptide by reacting adipic acid dihydrazide or carbohydrazide with carboxylic acid groups in the C-terminus or in aspartic or glutamic acid residues. An aminooxy group is incorporated on a peptide by reacting the N-terminus or a lysine residue with a heterobifunctional molecule containing an NHS ester on one end and a phthalimidoxy group on the other end, followed by cleavage with hydrazine. The reaction is, optionally, catalyzed by addition of aniline.

The cleavage rate of any linker is tuned, for example, by modifying the electron density in the vicinity of the cleavable link or by sterically affecting access to the cleavage site (e.g., by adding bulky groups, such as methyl groups, ethyl groups, or cyclic groups).

Cleavable linkers are, alternatively, generated using methods set forth in Bioconjugate Techniques, by Greg Hermanson, 3rd edition.

Installation of a thiol, amine, or aldehyde group in RNA or DNA, as a functional handle, is carried out as described above in EXAMPLE 8.

Any peptide oligonucleotide complexes of the present disclosure described in EXAMPLE 1-EXAMPLE 7 may contain a cleavable linker and are described. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 10

### Generation of Stable Linkers Between an Oligonucleotide and a Peptide

This example describes generation of a stable linkers between RNA, DNA, or any oligonucleotide, with any one of peptides of SEQ ID NO: 1-SEQ ID NO: 134 or SEQ ID NO: 306-SEQ ID NO: 335. A stable linker through a secondary amine is generated by reductive amination, achieved by combining an aldehyde-containing oligonucleotide with the amine at the N-terminus of a peptide or in a lysine residue, followed by reduction with sodium cyanoborohydride.

A stable amide linkage is generated by combining an amine group on an oligonucleotide with the carboxylate at the C-terminus of a peptide or in an aspartic acid or glutamic acid residues.

A stable carbamate linkage is generated by activating a hydroxyl group in an oligonucleotide with carbonyldiimidazole (CDI) or N,N′-disuccinimidyl carbonate (DSC) and subsequently reacted with a peptide's N-terminus or lysine residue.

A maleimide linker is created by combining a thiol-containing oligonucleotide with a maleimide functionalized peptide. The peptide is functionalized using an NHS-X-maleimide heterobifunctional agent on a reactive amine in the peptide, wherein X is any linker. A maleimide linker is used as a stable linker or as a slowly cleavable linker, which is cleaved by exchange with other thiol-containing molecules in biological fluids. The maleimide linker is also stabilized by hydrolyzing the succinimide moiety of the linker using various substituents, including those described in Fontaine et al., Bioconjugate Chem., 2015, 26 (1), pp 145-152.

Other methods of incorporating, adding, or modifying functional groups in polynucleotides, for example, are carried out using techniques set forth in Bioconjugate Techniques, by Greg Hermanson, 3rd edition.

Installation of a thiol, amine, or aldehyde group in an oligonucleotide, as a functional handle, is carried out as described above in EXAMPLE 8.

Any peptide oligonucleotide complexes of the present disclosure described in EXAMPLE 1-EXAMPLE 7 may contain a stable linker and are described. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 11

### Generation of an Enzyme Cleavable Linkage Between an Oligonucleotide and a Peptide

This example describes generation of an enzyme cleavable linkage between RNA, DNA, or any oligonucleotide, and any one of peptides of SEQ ID NO: 1-SEQ ID NO: 134 or SEQ ID NO: 306-SEQ ID NO: 335. An enzymatically cleavable linkage is generated between an oligonucleotide and a peptide. The conjugate with a cleavable linkage is administered in vitro or in vivo and is cleaved by enzymes in the cells or body, releasing the oligonucleotide. The enzyme is present in the endosome/lysosome, the cytosol, the cell surface, or is upregulated in the tumor microenvironment or the tissue microenvironment. These enzymes include, but are not limited to, cathepsins (such as all those listed in Kramer et al., Trends Pharmacol Sci. 2017 October; 38(10):873-898) such as cathepsin B, glucoronidases including beta-glucuronidase, hyaluronidase and matrix metalloproteases, such as MMP-1, 2, 7, 9, 13, or 14 (Kessenbrock et al., Cell. 2010 Apr. 2; 141(1): 52-67). Cathepsin or MMPs cleave amino acid sequences of any one of SEQ ID NO: 255, SEQ ID NO: 259, or SEQ ID NO: 271-SEQ ID NO: 296, shown below in TABLE 13 (see also Nagase, Hideaki. “Substrate specificity of MMPs.” Matrix Metalloproteinase Inhibitors in Cancer Therapy. Humana Press, 2001. 39-66; Dal Corso et al., Bioconjugate Chem., 2017, 28 (7), pp 1826-1833; Dal Corso et al., Chemistry-A European Journal 21.18 (2015): 6921-6929; Doronina et al., Bioconjug Chem. 2008 October; 19(10):1960-3). Glucuronidase linkers include any one of those described in Jeffrey et al., Bioconjugate Chem., 2006, 17 (3), pp 831-840.

TABLE 13
Enzymatically Cleavable Linkers

| | | May be |
|---|---|---|
| SEQ ID NO | Sequence | Cleaved By |
| SEQ ID NO: 255 | Val-Ala | Cathepsin |
| SEQ ID NO: 259 | Val-Lys | Cathepsin |
| SEQ ID NO: 271 | Val-Arg | Cathepsin |
| SEQ ID NO: 272 | Val-Cit | Cathepsin |
| SEQ ID NO: 273 | Phe-Lys | Cathepsin |
| SEQ ID NO: 274 | Met-Lys | Cathepsin |
| SEQ ID NO: 275 | Asn-Lys | Cathepsin |
| SEQ ID NO: 276 | Ile-Pro | Cathepsin |
| SEQ ID NO: 277 | Gly-Ile | MMP |
| SEQ ID NO: 278 | Gly-Leu | MMP |
| SEQ ID NO: 279 | Gly-Tyr | MMP |
| SEQ ID NO: 280 | Gly-Met | MMP |
| SEQ ID NO: 281 | Met-Ile | MMP |
| SEQ ID NO: 282 | Ala-Ile | MMP |
| SEQ ID NO: 283 | Pro-Ile | MMP |
| SEQ ID NO: 284 | Gly-Pro-Gln-Gly-Ile-Ala-Gly-Gln | MMP |
| SEQ ID NO: 285 | Gly-Pro-Gln-Gly-Ile-Phe-Gly-Gln | MMP |
| SEQ ID NO: 286 | Gly-Pro-Gln-Gly-Ile-Trp-Gly-Gln | MMP |
| SEQ ID NO: 287 | Gly-Pro-Gln-Gly-Ile-Leu-Gly-Gln | MMP |
| SEQ ID NO: 288 | Gly-Pro-Gln-Gly-Ile-Arg-Gly-Gln | MMP |
| SEQ ID NO: 289 | Gly-Pro-Leu-Gly-Ile-Ala-Gly-Gln | MMP |
| SEQ ID NO: 290 | Gly-Pro-Met-Gly-Ile-Ala-Gly-Gln | MMP |
| SEQ ID NO: 291 | Gly-Pro-Tyr-Gly-Ile-Ala-Gly-Gln | MMP |
| SEQ ID NO: 292 | GSVAGS | Cathepsin |
| SEQ ID NO: 293 | GGGGSVAGGGGS | Cathepsin |
| SEQ ID NO: 294 | GGGGSGGGGSVAGGGGSGGGGS | Cathepsin |
| SEQ ID NO: 295 | GGGGSGGGGSPLGLAGGGGGSGGGGS | |
| SEQ ID NO: 296 | AEAAAKEAAAKAVAAEAAAKEAAAKA | |

A Val-Cit-PABC enzymatically cleavable linker, such as described in Jain et al., Pharm Res. 2015 November; 32(11):3526-40, is created by conjugating the PABC end to an amine group on the oligonucleotide. The valine end is further linked to the peptide, for example, by generating an amide bond to the C-terminus of the peptide. A spacer on either side of the molecule is optionally incorporated in order to facilitate steric access of the enzyme to the Val-Cit linkage (SEQ ID NO: 272). The linkage to the peptide is, alternatively, generated by activating the N-terminus of the peptide with SATA and creating a thiol group, which is subsequently reacted to a maleimidocaproyl group attached to the N-terminus of the Val-Cit pair (SEQ ID NO: 272). Upon cleavage by cathepsin B, the self-immolative PABC group spontaneously eliminates, releasing the amine-containing oligonucleotide with no further chemical modifications. Other amino acid pairs include Glu-Glu, Glu-Gly, and Gly-Phe-Leu-Gly.

Installation of a thiol, amine, or aldehyde group in RNA or DNA, as a functional handle, is carried out as described above in EXAMPLE 8.

Any peptide oligonucleotide complexes of the present disclosure described in EXAMPLE 1-EXAMPLE 7 may contain an enzyme cleavable linker and are described. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 12

### Conjugation of an Oligonucleotide and a Peptide

This example describes conjugation of an oligonucleotide to a peptide of the present disclosure. The peptide is SEQ ID NO: 32. The N-terminus of SEQ ID NO: 32 is conjugated via reductive amination to 4-formyl-PBA. The PBA-containing peptide is complexed to the 3′ diol group of the oligonucleotide to form a boronate ester.

Alternatively, the oligonucleotide has a thiol-containing or phosphorothioate-containing nucleotide residue included in the sequence, during synthesis. The N-terminus of SEQ ID NO: 32 is modified with SATA (with subsequent deprotection using hydroxylamine) to form a thiol group.

Alternatively, The N-terminus of SEQ ID NO: 32) is modified with SPDP-PEG₄-NHS ester to form a protected thiol group, with a flexible hydrophilic PEG spacer. The two thiol groups in the modified oligonucleotide and SEQ ID NO: 32 are combined to form a cleavable disulfide bond. Alternatively, The N-terminus of SEQ ID NO: 32 is modified with bromoacetamido-PEG₄-TFP ester to form an amide bond, and then reacted with the thiol group within the oligonucleotide, to form a stable thioether bond.

Alternatively, the oligonucleotide has an amine-containing nucleotide included in the sequence, during synthesis. The N-terminus of SEQ ID NO: 32 is modified with SATA to form a thiol group. A maleimidocaproyl-Val-Cit-PABC linker is conjugated to the amine in the oligonucleotide and to the thiol in SEQ ID NO: 32.

Alternatively, the oligonucleotide is conjugated to the N-terminus of SEQ ID NO: 32 via reductive amination after oxidation of the 3′ diols to form a secondary amine conjugate.

Alternatively, the oligonucleotide has the 3′ end oxidized to aldehydes via periodate oxidation. The aldehyde is then reacted with the peptide of SEQ ID NO: 32, which is functionalized with an aminooxy group on the N-terminus to form a cleavable oxime bond.

Alternatively, a dsRNA is used. The 3′ end of the sense strand is synthesized with a thiol modification as shown in FIG. 3. The N-terminus of SEQ ID NO: 32 is modified with bromoacetamido-PEG₄-TFP ester to form an amide bond, and then reacted with the thiol group within the dsRNA to form a stable thioether bond. Alternatively, the 5′ end of the sense strand or amino terminated nucleotides serves as the site of modification.

Alternatively, the peptide is any one of SEQ ID NO: 1-SEQ ID NO: 134 or SEQ ID NO: 306-SEQ ID NO: 335.

Alternatively, rather than using the N-terminus of the peptide, a lysine residue in the peptide is used. Optionally, one or more or all of the lysine residues are mutated to arginine residues so only one, or no, lysine residues are available for amine-based reactions.

Installation of a thiol, amine, or aldehyde group in RNA or DNA, as a functional handle, is carried out as described above in EXAMPLE 8.

Optionally, the oligonucleotide is synthesized using any one or more modified bases in order to alter the stability, tissue biodistribution, immune reactivity, or any other property of the oligonucleotide.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

### Example 13

### Surface Plasmon Resonance (SPR) Analysis of Peptide Binding Interactions

This example illustrates surface plasmon resonance (SPR) analysis of peptide-oligonucleotide conjugate binding interactions with TfR.

A peptide-oligonucleotide conjugate (also referred to herein as a peptide oligonucleotide complex) is constructed by combining any of the TfR-binding peptides of this disclosure with an oligonucleotide. Optionally, the oligonucleotide is designed for RNase H-engaging antisense, splice-blocking antisense, siRNA, anti-miR, U1 adapter, or aptamer therapy. Optionally, a stable or cleavable linker is used between the peptide and the oligonucleotide.

The peptide-oligonucleotide conjugate is subjected to SPR (surface plasmon resonance) analysis. The affinity of the peptide-oligonucleotide to TfR is measured by SPR, using either a TfR moiety or the peptide-oligonucleotide conjugate as the analyte. Optionally the TfR is human, murine, rat, canine, or non-human primate (e.g., cynomolgus or rhesus). The kₒₙ, k_{off}, and/or K_{D} of the peptide-oligonucleotide conjugate for TfR is measured. Optionally the kₒₙ, k_{off}, and K_{D} of the peptide alone (not conjugated to the oligonucleotide) to TfR is also measured. The K_{D} of the peptide-oligonucleotide conjugate to TfR is found to be adequate to bind to the desired target cell and drive endocytic uptake or transcytosis of the peptide-oligonucleotide conjugate. The K_{D} of the peptide-oligonucleotide conjugate to the TfR is optionally found to be less than 1 μM, less than 100 nM, less than 10 nM, less than 1 nM, or less than 0.5 nM. Optionally, the K_{D} of the peptide-oligonucleotide conjugate to the TfR is found to be similar to the K_{D} of the peptide alone, such as within 100-fold, 10-fold, 5-fold, or 2-fold of each other. Optionally, the k_{off} of the peptide-oligonucleotide conjugate is found to be sufficient to allow peptide-oligonucleotide conjugate uptake into the endosome and release from TfR prior to recycling or release from TfR after transcytosis. In some cases, an increased TfR-binding affinity can correspond to a reduced transcytosis function, wherein in some cases, an increased TfR-binding affinity does not correspond to a change in transcytosis function compared to the reference peptide. It is assumed that the ratio of kₒₙ/k_{off} can affect the transcytosis function of a peptide, and thus modulation of kₒₙ and/or k_{off} can be used to generate TfR-binding peptides with optimal TfR binding affinity and transcytosis function. Optionally, the linker between the peptide and the oligonucleotide and/or the oligonucleotide or peptide sequence are changed such that the kₒₙ, k_{off}, and/or K_{D} of the modified peptide-oligonucleotide conjugate to TfR is closer to the desired values. Optionally, different peptide-oligonucleotide conjugates are compared, and the peptide-oligonucleotide conjugate with the most desirable kₒₙ, k_{off}, and/or K_{D} is selected for further use.

### Example 14

### Cross Reactivity of TfR-Binding Peptides to Murine TfR

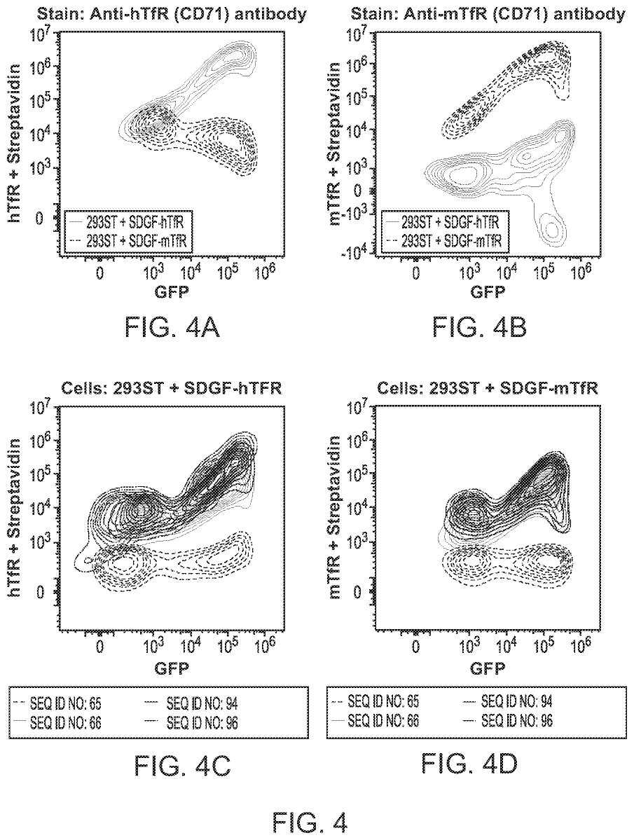

This example illustrates cross reactivity of TfR-binding peptides of the present disclosure to murine TfR in cell surface binding assays. Briefly, 293F cells expressing either human or mouse TfR on their surface were stained with soluble TfR-binding peptides that were directly labeled with AlexaFluor 647 dye via NHS-ester conjugation. FIG. 4A and FIG. 4B show flow cytometry plots that verify human versus mouse TfR expression using species-specific antibodies. FIG. 4C and FIG. 4D demonstrate that the peptides effectively bind to both homologs. In a similar experiment, flow cytometry was used to demonstrate effective binding of SEQ ID NO: 65, SEQ ID NO: 66, SEQ ID NO: 96, and Anti-Tf antibodies (positive controls). SDGF surface expression of the human and murine TfR ectodomains (HsTfR and MmTfr, respectively) was confirmed by species-specific antibodies. Staining of these cells with Alexa Fluor 647-labeled CDP variants (e.g., SEQ ID NO: 65, SEQ ID NO: 66, or SEQ ID NO: 67) demonstrated cross-reactivity and improved staining of higher affinity variants.

Any of peptide within the peptide oligonucleotide complex of the present disclosure (e.g., any one of SEQ ID NO: 1-SEQ ID NO: 134 or SEQ ID NO: 306-SEQ ID NO: 335) can be modified at one or more of the corresponding residues identified in the crystal structure above, to generate peptide variants with improved properties including enhanced stability and increased (or decreased) binding properties or modified TfR-binding affinity, and/or increased (or decreased) transcytosis function. Without being bound by any theory, the herein described data indicate that all amino acid residues of a TfR-binding peptide may be important for cross-reactivity. Cross-reactivity was confirmed in flow cytometry binding assays (FIG. 4).

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18. These peptide oligonucleotide complexes can exhibit enhanced stability and increased (or decreased) binding properties or modified TfR-binding affinity, and/or increased (or decreased) transcytosis function.

### Example 15

### Whole Body Autoradiography of TfR-Binding Peptides

This example illustrates the determination of biodistribution for the radiolabeled peptides having a sequence of SEQ ID NO: 65 and SEQ ID NO: 96 (SEQ ID NO: 65 and SEQ ID NO: 96 are SEQ ID NO: 1 and SEQ ID NO: 32, respectively, with an added N-terminal GS) acquired via autoradiography in vivo.

Each peptide was radiolabeled by methylating lysines and the N-terminus as described in EXAMPLE 19. As such, the peptide may contain methyl or dimethyl lysines and a methylated or dimethlyated amino terminus. A dose of 100 nmol radiolabeled peptide was administered via tail vein injection in Female Harlan athymic nude mice, weighing 20-25 g. ¹⁴C labeled CDPs were resuspended to 100 nmol in 100 μL PBS for intravenous injection into mice.

Each radiolabeled peptide was allowed to freely circulate within the animal for the described time period (30 minutes or 3 hours) before the animals were euthanized and sectioned.

Whole body autoradiography (WBA) sagittal sectioning was performed as follows. At the end of the dosing period, mice were frozen in a hexane/dry ice bath and then embedded in a frozen block of carboxymethylcellulose. Frozen carcasses were allowed to off gas hexane overnight at −20° C. prior to embedding in chilled 2% carboxymethylcellulose (Sigma Aldrich, C5013). Holes were then drilled into each block and radioactive ¹⁴C glycine controls (0.5 μCi mL⁻¹, American Radiolabeled Chemicals) in PBS+0.5% BSA were pipetted in and allowed to freeze, prior to sectioning (40 μm) on a H/I Bright 8250 Cryostat (Hacker Instruments). Whole animal sagittal slices were prepared that resulted in thin frozen sections for imaging. Thin frozen sections were obtained using a microtome and allowed visualization of tissues such as brain, tumor, liver, kidney, lung, heart, spleen, pancreas, muscle, adipose, gall bladder, upper gastrointestinal tract, lower gastrointestinal tract, bone, bone marrow, reproductive tract, eye, cartilage, stomach, skin, spinal cord, bladder, salivary gland, and more. Sections were collected onto 4-inch wide tape (Scotch 821, ULINE) at 2-6 depths to sample all of the tissues of interest, and freeze dried in the cryostat for 2-3 days, after which they were mounted on sturdy paper and covered with cellophane. Mounted sections were exposed to storage phosphor plates (Raytest) along with a radioactive standard curve (146S-PL, American Radiolabeled Chemicals) for 7 days. Sections were allowed to desiccate in a freezer prior to imaging.

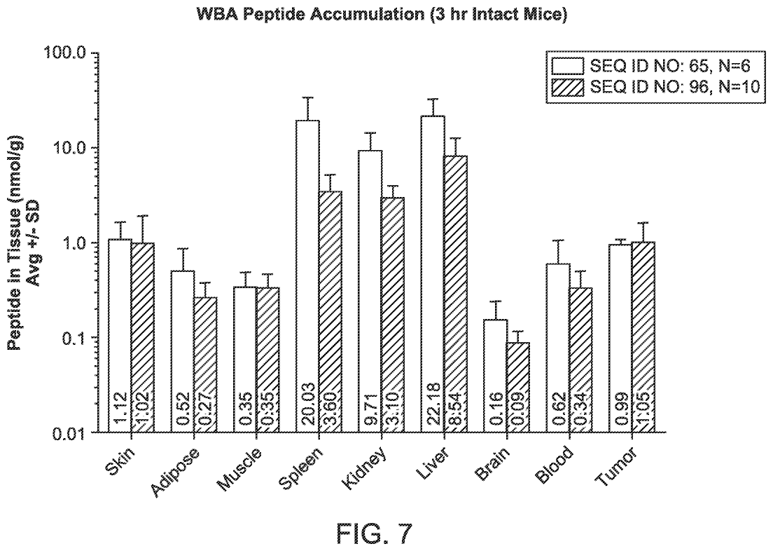

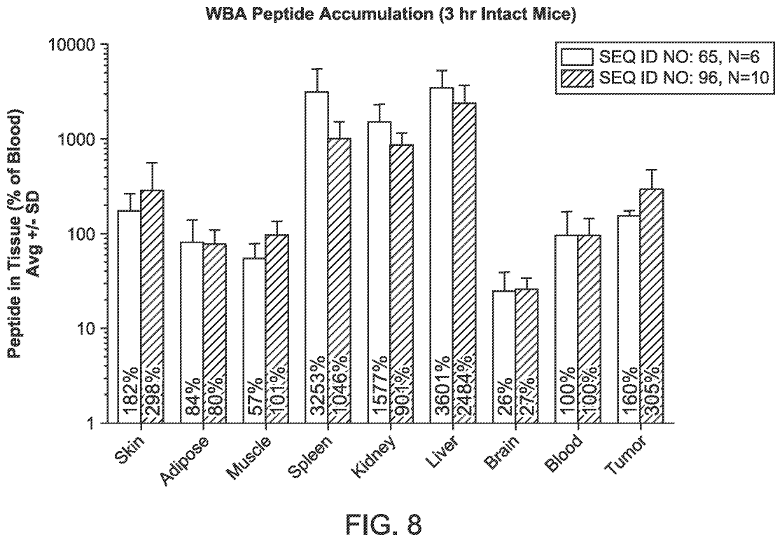

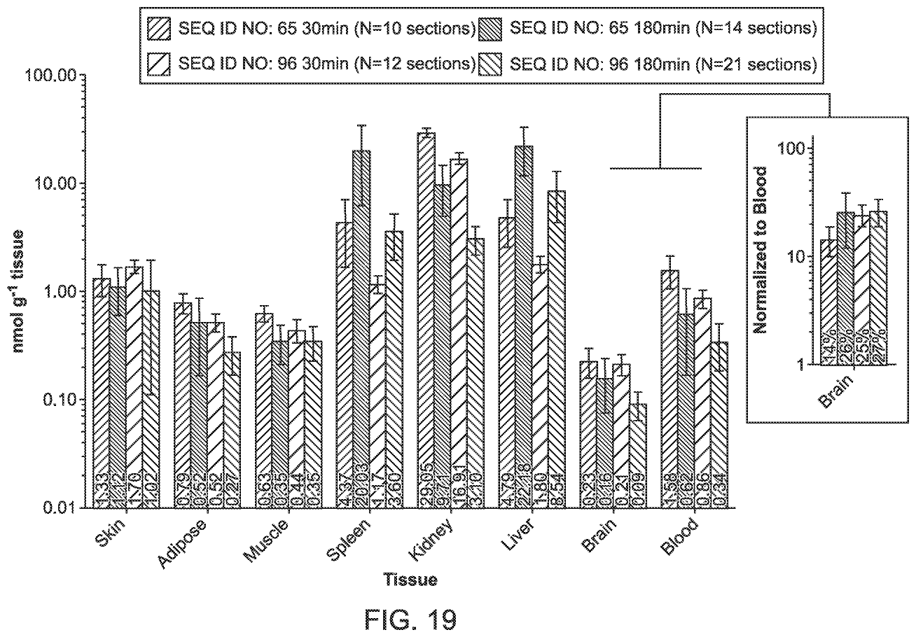

For the autoradiography imaging, tape mounted thin sections were freeze dried and radioactive samples were exposed to phophoimager plates for 7 days. These plates were developed and the signal (densitometry) from each organ was normalized to the signal found in the cardiac blood of each animal. A signal in tissue darker than the signal expected from blood in that tissue indicates accumulation in a region, tissue, structure, or cell. Scanning took place on a Raytest CR-35 imager at the “25 μm sensitive” setting. Quantitation was performed by background-adjusted densitometry using AIDA Image Analyzer v5.1 Whole Body Autoradiography Professional software (Raytest). For quantification, all region of interest measurements from a single tissue within a single section were averaged, adjusting for area. Blood values were assessed in the heart. Reporting values in nmol g⁻¹ tissue required calibration to the commercial standard strips co-exposed with each WBA sample set. The strips were themselves calibrated so that a known amount of radioactive counts per minute (CPM) per gram of tissue, determined by liquid scintillation counting (LSC), corresponded to the measured WBA densitometry at 11 standard strip concentration data points (linear range corresponding to 6.3 nCi g⁻¹ to 3.3 μCi g⁻¹). This calibration, combined with the peptides' known concentrations and specific activities (determined by liquid scintillation counting), permitted the conversion to nmol g⁻¹. For the quantitation shown in FIG. 7, FIG. 8, and FIG. 19, total number of sections analyzed is indicated in the key; not every section contained every tissue, but every tissue quantitation represents 1-3 regions of interest per section over 1-4 sections per mouse from 3-10 mice. Of note, at the 100 nmol dose and average mouse weight of 25 g, 4 nmol/g represents “100% injected dose”. Some tissues (spleen/kidney/liver) exceed this value because the peptide accumulates at the tissue beyond that expected by simple whole-body diffusion. This can indicate that the peptide preferentially accumulates in those tissues. Therefore, calculating a percent injected dose remaining in non-serum tissues can lead to uneven tissue distribution numbers, since there is no tissue-specific “time zero” amount.

FIG. 5 shows autoradiography images of mice that were administered with TfR-binding peptides having a sequence of SEQ ID NO: 65 (FIG. 5A), SEQ ID NO: 66 (FIG. 5B), SEQ ID NO: 94 (FIG. 5C), and SEQ ID NO: 96 (FIG. 5D). Whole body autoradiography was used with IV-injected ¹⁴C-labeled peptides to assess biodistribution. Images were taken 3 hours after dose with 100 nmol drug (˜20 mg/kg in ˜25 g mice). The images demonstrate substantial accumulation in spleen, liver, kidney, and also high accumulation in muscle, bone marrow, and skin. CNS accumulation was less than other tissues but substantial compared to serum levels (>25% of serum). Background blood levels were found in brain to be 3% of the cardiac blood signal.

FIG. 6 illustrates whole body autoradiography of a mouse injected intravenously with ¹⁴C-labeled peptides (SEQ ID NO: 65 and SEQ ID NO: 96) to assess biodistribution. Substantial accumulation in flank tumors was also seen. FIG. 6A illustrates white light images of mice injected intravenously with ¹⁴C-labeled SEQ ID NO: 65. FIG. 6B illustrates white light images of mice injected intravenously with ¹⁴C-labeled SEQ ID NO: 65. FIG. 6C illustrates whole body autoradiography of mice injected intravenously with ¹⁴C-labeled SEQ ID NO: 96. FIG. 6D illustrates whole body autoradiography of mice injected intravenously with ¹⁴C-labeled SEQ ID NO: 96. Notably, U87 tumor distribution of both peptides is complete as shown by significant levels of peptide throughout the flank tumor on the animal's back, even though the tumors appear poorly vascularized in the white light images to the right. The tumors were subcutaneous U87 brain tumors.

In addition to autoradiography, ex vivo scintillation counting was performed using the peptides having a sequence of SEQ ID NO: 65 and SEQ ID NO: 96 (FIG. 7, FIG. 8, and FIG. 19). FIG. 7 and FIG. 8 shows bar graphs of organ uptake data normalized to radiolabeled control samples in block and to peptide specific activity given in nmol/g levels, and only normalized to % of serum levels, respectively. Peptide specific activity given in nmol/g levels at both 30 min and 3 hour timepoints is show in FIG. 19. FIG. 7 illustrates the quantitation of peptide biodistribution and organ accumulation after a single 20 mg/kg IV dose of either a TfR-binding peptide having a sequence of SEQ ID NO: 65 or a peptide having a sequence of SEQ ID NO: 96. This dose achieved levels of >150 nM in the CNS, ˜1 μM in tumor after 3 hours. FIG. 8 illustrates the quantitation of peptide biodistribution and organ accumulation for the two peptides comprising SEQ ID NO: 65 and SEQ ID NO: 96, respectively. The values are shown as Percent of Blood Levels, which is a common BBB penetration metric (3% baseline). The data shows that for the TfR-binding peptides having a sequence of SEQ ID NO: 65 and SEQ ID NO: 96 biodistribution and organ accumulation for the peptides occurred in spleen, liver, kidney, skin, bones, and brain.

For the brain, the mice were not perfused prior to sectioning. Therefore, CNS capillaries can remain full of blood, and any peptide still in circulation can create a weak signal in the brain. However, it is generally accepted that CNS blood/capillaries occupy roughly 3% of the brain volume, and thus a signal of 3% vs blood may be considered equal to “no CNS accumulation.” Any signal above 3% is considered evidence of peptide CNS accumulation, either stuck in capillary cells or actual BBB penetration. Thus, at >25% blood levels, both peptides demonstrate substantial CNS accumulation by this metric.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18. These peptide oligonucleotide complexes can exhibit CNS accumulation or BBB penetration or accumulation in tissues that express TfR.

### Example 16

### Quantification of TfR Binding Peptide Accumulation in Tissues by Scintillation Counting

This example illustrates quantification of peptide in serum in tissues by scintillation counting. Peptides were radiolabeled by the methods described in EXAMPLE 19 and then administered intravenously to female Harlan athymic nude mice with intact kidneys. Radiolabeled peptides were administered intravenously at a dose of 100 nmol (14.7 Ci, 20 mg/kg). Mice were euthanized at various time points by CO₂ asphyxiation and tissues and biological fluids were collected including kidney, liver, brain, spleen, muscle, and skin. Solid tissues were homogenized prior to liquid scintillation counting. Quantitation of the amount of peptide in tissue (nmol/g) was performed by referencing peptide's molecular weight and specific ¹⁴C activity. FIG. 9 shows quantification of tissue accumulation determined by scintillation counting after administration of a peptide having a sequence of SEQ ID NO: 96 (SEQ ID NO: 96 is SEQ ID NO: 32 with an added N-terminal GS). FIG. 9A illustrates scintillation counting over time in the kidneys. FIG. 9B illustrates scintillation counting over time in the liver. FIG. 9C illustrates scintillation counting over time in the brain (CNS). FIG. 9D illustrates scintillation counting over time in the spleen. FIG. 9E illustrates scintillation counting over time in the muscle. FIG. 9F illustrates scintillation counting over time in the skin. The data shows that for the peptide having a sequence of SEQ ID NO: 96 accumulation of peptides was confirmed in various tissues including spleen, liver, kidney, skin, bones, and brain. Over the time course of 64 hours, the amount of peptide having a sequence of SEQ ID NO: 96 significantly decreased in tissues such as kidney, liver, spleen, and muscle starting about 1-2 hours after administration, wherein the amount of peptide in the brain slightly decreased but then stabilized over the course of the experiment. SEQ ID NO: 96 demonstrated biphasic plasma elimination kinetics, with a first elimination half-life of 15.6 minutes. The highest levels are seen in the kidney, liver, and spleen; the kidney is responsible for excretion of small plasma solutes, while both hepatic and splenic tissues reportedly express high levels of TfR. The brain showed smaller but measurable levels.

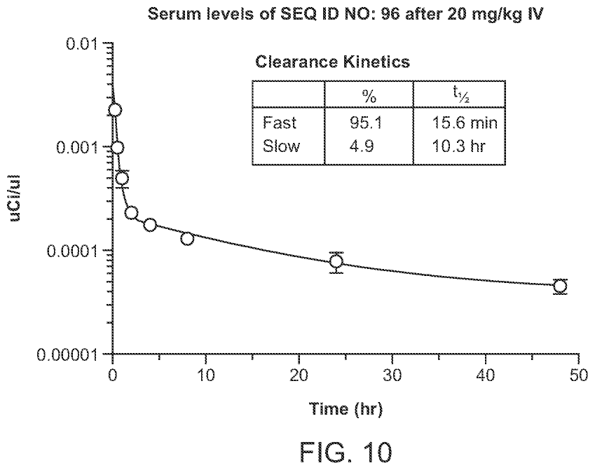

Elimination of a peptide of SEQ ID NO: 96 was also tested in female Harlan athymic nude mice. SEQ ID NO: 96 was intravenously administered at a dose of 100 nmol (14.7 μCi, 20 mg/kg). FIG. 10 illustrates a serum elimination plot, including two-phase elimination regression analysis showing fast (95%, 15.6 min t_{1/2}) and slow (5%, 10.3 hr t_{1/2}) phase kinetics in mice intravenously administered SEQ ID NO: 96.

This data demonstrates that the uptake and retention of these peptides in organs such as spleen, liver, kidney, skin, bones, and brain allows effective targeting of and delivery of active and/or detectable agents to these organs using the TfR-binding peptides of the present disclosure.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18. These peptide oligonucleotide complexes can exhibit effective targeting of and delivery of active and/or detectable agents.

### Example 17

### Multiple Time Regression and Capillary Depletion Analysis of Peptides

This example illustrates multiple time regression (MTR) and capillary depletion analysis of peptides of the present disclosure.

Mice (strain CD-1, N=1 per time point) were anesthetized and the left jugular vein and right carotid arteries were surgically exposed. Labeled peptides (0.45 μCi in 200 μL volume; specific activities were 96 Ci/mol for SEQ ID NO: 65 and 147 Ci/mol for SEQ ID NO: 96 (SEQ ID NO: 65 and SEQ ID NO: 96 are SEQ ID NO: 1 and SEQ ID NO: 32, respectively, with an added N-terminal GS), according to methods described in EXAMPLE 19) were injected into the jugular vein. After a given time within a time course (0-30 minutes), arterial blood was collected, followed by decapitation and brain collection. Brains and serum were tested via liquid scintillation counting for ¹⁴C levels, and the data was incorporated into a multiple time regression mathematic model to quantify CNS accumulation rate. To determine whether this CNS accumulation is limited to capillaries or actually represents vascular transcytosis, a separate set of animals (N=3/peptide) were euthanized 5 minutes after dosage, and brain homogenates were subject to centrifugation through a dextran density gradient. This allowed separation of capillaries from the parenchyma, and the two tissues were separately subjected to scintillation counting to quantify the ¹⁴C signal. Normalized signal ratios were an indicator of BBB penetration; for example, SEQ ID NO: 65 exhibited a substantially higher signal in the parenchyma than the capillaries, which demonstrated transcytosis across the BBB.

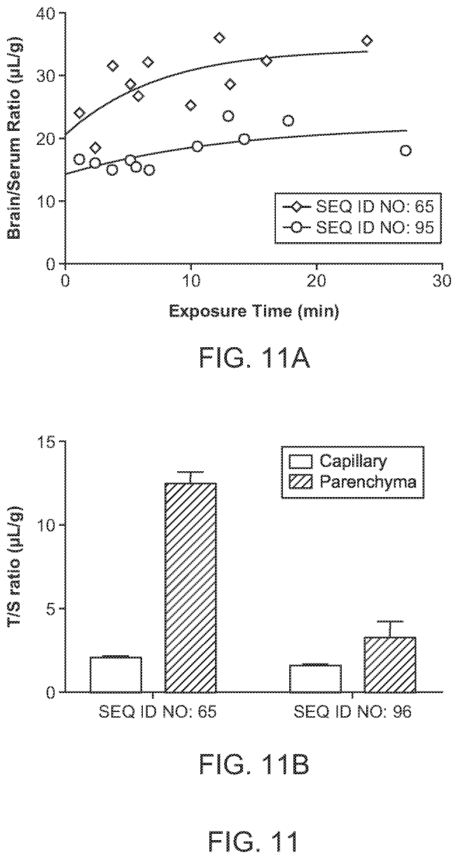

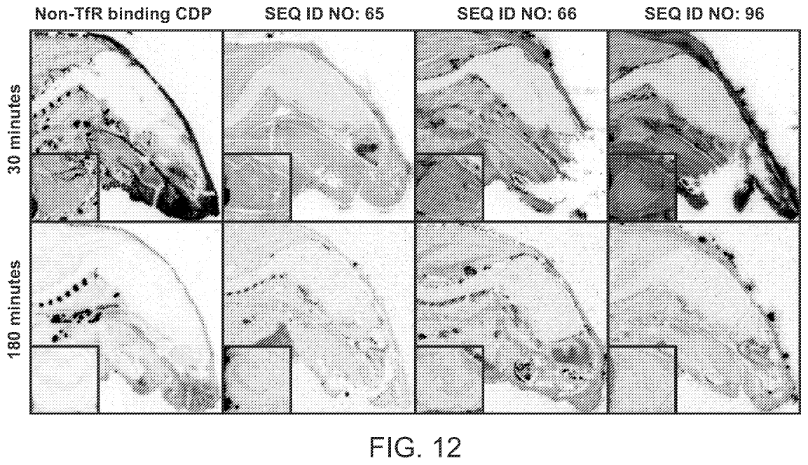

FIG. 11 illustrates multiple time regression analysis and capillary depletion analysis of TfR-binding peptides having a sequence of SEQ ID NO: 65 and SEQ ID NO: 96. FIG. 11A illustrates multiple regression analysis of SEQ ID NO: 65 and SEQ ID NO: 96 in a single plot, wherein the y-axis indicates the brain to serum ratio. FIG. 11B illustrates parenchyma and capillary distribution of TfR-binding peptides having a sequence of SEQ ID NO: 65 and SEQ ID NO: 96, wherein the y-axis indicates the tissue-to-serum (T-to-S) ratio (μL/g), that is the ratio of radioactivity measured in the respective tissue (in this case, parenchyma or capillaries) to the radioactivity measured in circulating serum at the same time point.

The bar graph of FIG. 11B, in particular, strongly suggests BBB transport and parenchyma access of a peptide of SEQ ID NO: 65, demonstrating the peptides of the present disclosure are in fact capable of promoting TfR-mediated transport across the BBB (e.g., vesicular transcytosis). Any peptide within the peptide oligonucleotide complex of the present disclosure (e.g., any one of SEQ ID NO: 1-SEQ ID NO: 134 or SEQ ID NO: 306-SEQ ID NO: 335) can be similarly capable of promoting TfR-mediated transport across the BBB (e.g., via vesicular transcytosis). The short time point for the capillary depletion experiment (30 mins) likely explains lack of obvious transcytosis of SEQ ID NO: 96 observed in this assay, but CRE-Luc reporter in mice was successfully induced (4 hr timepoint).

This data demonstrates that the peptides of the present disclosure enable TfR-mediated transport enabling the delivery of therapeutic and/or diagnostic agents into tissues expressing TfR such as brain (transcytosis via the BBB), tumors, bone and bone marrow, and immune cells. The transport can occur across cell layers such as endothelial cells (e.g., an intact BBB) or across cell membranes. Thus, the peptides of the present disclosure can be used as effective delivery vehicles for a variety of active agents into any cell, tissue, or organ that expresses TfR. In addition, this data demonstrates binding of the herein described peptides to murine TfR. Accessing and specifically targeting tumors behind an intact BBB may be a particular advantage (e.g., high unmet clinical need) of the herein described TfR-binding peptides compared to conventional methods.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18. These peptide oligonucleotide complexes can exhibit CNS accumulation or BBB penetration, and specifically targeting tumors behind an intact BBB may be a particular advantage.

### Example 18

### Extension of Peptide Oligonucleotide Complex Plasma Half-Life

This example demonstrates a method of extending the serum or plasma half-life of a peptide as disclosed herein. A peptide oligonucleotide complex having a peptide sequence of any one of SEQ ID NO: 1-SEQ ID NO: 134 or SEQ ID NO: 306-SEQ ID NO: 335 is modified (such as modified in the peptide, the oligonucleotide, the linker, or either end) in order to increase its plasma half-life. Conjugation of the peptide oligonucleotide complex to a near infrared dye, such as Cy5.5 is used to extend serum half-life of the peptide construct. Alternatively, conjugation of the peptide oligonucleotide complex to an albumin binder, such as Albu-tag or a C₁₄-C₁₈ fatty acid, is used to extend plasma half-life. Optionally, plasma half-life is extended as a result of reduced immunogenicity by using minimal non-human protein sequences.

### Example 19

Peptide Radiolabeling with ¹⁴C

This example describes the radiolabeling of peptides with ¹⁴C. Peptides of the present disclosure were radiolabeled by reductive methylation with ¹⁴C formaldehyde and sodium cyanoborohydride using standard techniques (such as those described in Jentoft et al. J Biol Chem. 254(11):4359-65. 1979). Briefly, peptides (10 mg) were dissolved in 3.2 mL water with 470 μL 10×PBS. 0.72 mCi (12.6 μM) ¹⁴C-formaldehyde (57 Ci mol⁻¹, Pharmaron) and sodium cyanoborohydride (to 100 mmol, in water) were added, followed by vortexing for 15 seconds. The reactions were incubated at RT overnight. 10 μL reaction was set aside in 1 mL water for analysis, after which the ¹⁴C methylated peptide was purified on a Strata-X reversed-phase column (30 mg, Phenomenex, washed with 3 mL methanol and 3 mL water). After loading, the column was washed with 4 mL water and eluted with 4 mL 2% formic acid in methanol. Labeled CDPs were dried in a blow-down evaporator (40° C. under nitrogen stream) and stored at 18° C. until use. Specific activity was determined by scintillation counting. Variant-specific activities of the variants of SEQ ID NO: 65 were as follows: SEQ ID NO: 65: 32 Ci/mol for 180 minute animals, 187 Ci/mol for 30 minute animals. SEQ ID NO: 66: 260 Ci/mol for 180 minute animals, 179 Ci/mol for 30 minute animals. SEQ ID NO: 96: 273 Ci/mol for 180 minute animals, 147 Ci/mol for 30 minute animals. The sequences were engineered to have the amino acids, “G” and “S” at the N terminus. See Methods in Enzymology V91:1983 p.570 and JBC 254(11):1979 p. 4359. An excess of formaldehyde was used to drive complete methylation (dimethylation of every free amine).

### Example 20

### Peptide Oligonucleotide Complex Detectable Agent Conjugates

This example describes the dye labeling of peptide oligonucleotide complexes. The peptide and the oligonucleotide of a peptide oligonucleotide complex of the disclosure are produced as described herein and conjugated together with a linker. The peptide nucleotide complex, optionally via the N-terminus or a lysine residue of the peptide, is conjugated to, linked to, or fused to a detectable agent via an activated ester (e.g., NHS ester) in the presence of either DCC or EDC to produce a peptide-detectable agent conjugate. The detectable agent can comprise an ultraviolet (UV) dye, a blue dye, or both. Exemplary UV and blue dyes for fluorophores include: ALEXA FLUOR 350 and AMCA dyes (e.g., AMCA-X Dyes), derivatives of 7-aminocoumarin dyes, dialkylaminocoumarin reactive versions of ALEXA FLUOR 350 dyes, ALEXA FLUOR 430 (and reactive UV dyes that absorb between 400 nm and 450 nm have appreciable fluorescence beyond 500 nm in aqueous solution), Marina Blue and Pacific Blue dyes (based on the 6,8-difluoro-7-hydroxycoumarin fluorophore), exhibit bright blue fluorescence emission near 460 nm, hydroxycoumarin and alkoxycoumarin derivatives, Zenon ALEXA FLUOR 350, Zenon ALEXA FLUOR 430 and Zenon Pacific Blue, succinimidyl ester of the Pacific Orange dye, Cascade Blue acetyl azide and other pyrene derivatives, ALEXA FLUOR 405 and its derivatives, pyrene succinimidyl esters, Cascade Yellow dye, PyMPO and pyridyloxazole derivatives, aminonaphthalene-based dyes and dansyl chlorides, dapoxyl dyes (e.g., Dapoxyl sulfonyl chloride, amine-reactive Dapoxyl succinimidyl ester, carboxylic acid-reactive Dapoxyl (2-aminoethyl)sulfonamide), bimane dyes (e.g., bimane mercaptoacetic acid) and its derivatives, NBD dyes and its derivatives, QsY 35 dyes and its derivatives, fluorescein and its derivatives. The marking dye can comprise an infrared dye, near infrared dye or both. Exemplary infrared and near infrared dyes for fluorophores include: DyLight-680, DyLight-750, VivoTag-750, DyLight-800, IRDye-800, VivoTag-680, Cy5.5, or an indocyanine green (ICG) and any derivative of the foregoing, cyanine dyes, acradine orange or yellow, ALEXA FLUORs and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are known and described in international patent application no. PCT/US2014/77.

The peptide oligonucleotide complex detectable agent conjugates are administered to a subject. The subject can be a human or a non-human animal.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18. These peptide oligonucleotide complexes can exhibit CNS accumulation or BBB penetration or endocytosis in TfR-containing cells, and specifically targeting tumors behind an intact BBB may be a particular advantage, including wherein the detectable agent is a fluorophore, with respect to diagnostic or fluorescence guided surgical techniques.

In a similar manner a detectable agent (e.g., a near-infrared dye, a contrast agent, a nanoparticle, a metal-containing nanoparticle, a metal chelate, an X-ray contrast agent, a PET agent, a radionuclide, or a radionuclide chelator) can be complexed with the peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335). Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18. These peptide oligonucleotide complexes can exhibit CNS accumulation or BBB penetration or endocytosis in TfR-containing cells, and specifically targeting tumors behind an intact BBB may be a particular advantage, with respect to radiodiagnostic or radiotherapeutic techniques.

### Example 21

### Peptide Oligonucleotide Complex Active Agent Conjugates

This example describes the peptide oligonucleotide complex active agent conjugates. The peptide and the oligonucleotide of a peptide oligonucleotide complex of the disclosure are expressed recombinantly or chemically synthesized and then conjugated together with a linker. Then the peptide nucleotide complex, optionally via N-terminus or a lysine residue of the peptide is conjugated to, linked to, or fused to an additional active agent via an activated ester (e.g., NHS ester) in the presence of either DCC or EDC to produce a peptide-active agent conjugate. The additional active agent may be an additional nucleotide active agent, or the additional active agent may be a small molecule, peptide, or protein active agent.

The peptide oligonucleotide complex active agent conjugates are administered to a subject in need thereof. The subject can be a human or a non-human animal. After administration, the peptide oligonucleotide complex active agent conjugates transcytoses into an organ across an endothelial barrier via TfR-mediated transcytosis. The endothelial barrier is the blood brain barrier (BBB). The peptide active agent conjugate ameliorates the condition the subject is being treated for, for example brain cancer.

### Example 22

### Mutation of Peptide Oligonucleotide Complexes to Improve Binding Affinity

This example illustrates mutation of peptides within the peptide oligonucleotide complexes to decrease the off-rate and/or increase the on-rate of receptor binding.

A peptide of the present disclosure is mutated using Site Saturation Mutagenesis (SSM) or any random mutagenesis method to increase or decrease the off rate and/or increase the on rate, thereby increasing or decreasing the binding affinity. The peptide within the peptide nucleotide complex is selected from any one of the peptides shown in SEQ ID NO: 1-SEQ ID NO: 134 or SEQ ID NO: 306-SEQ ID NO: 335. The rate of delivery of the peptide to the target tissue, such as the brain parenchyma, is measured to determine the optimal binding affinity for the peptide within the peptide nucleotide complex for the given application.

### Example 23

### Treatment of Cancer

This example illustrates treatment of cancer using peptide nucleotide complexes of the present disclosure. The peptide and the oligonucleotide of a peptide nucleotide complex of the present disclosure are recombinantly expressed or chemically synthesized and then conjugated together with a linker. The peptide nucleotide complex administrated in a pharmaceutical composition to a subject in need thereof as a therapeutic for cancer. The peptide within the peptide oligonucleotide complex is selected from any one of the peptides having a sequence of SEQ ID NO: 1-SEQ ID NO: 134 or SEQ ID NO: 306-SEQ ID NO: 335. One or more peptide nucleotide complexes are administered to a subject. The subject can be a human or an animal.

The pharmaceutical composition is administered intravenously, subcutaneously, intramuscularly, intrathecally, intratumorally, intravitreally, or orally. The cancer to be treated with a peptide nucleotide complex as described herein can be a brain cancer or a CNS-related cancer.

### Example 24

### Treatment of Bladder Cancer

This example illustrates treatment of cancer using peptide oligonucleotide complexes of the present disclosure. The peptide and the oligonucleotide of a peptide oligonucleotide complex of the present disclosure are recombinantly expressed or chemically synthesized and then conjugated together with a linker. The peptide oligonucleotide complex administrated in a pharmaceutical composition to a subject in need thereof as a therapeutic for cancer. The peptide within the peptide nucleotide complex is selected from any one of the peptides having a sequence of SEQ ID NO: 1-SEQ ID NO: 134 or SEQ ID NO: 306-SEQ ID NO: 335. One or more peptide oligonucleotide complexes are administered to a subject. The subject can be a human or an animal. The pharmaceutical composition is administered by intravesical administration using a catheter to the bladder, or is administered intravenously, subcutaneously, intramuscularly, intrathecally, intratumorally, intravitreally, or orally. The cancer to be treated with a peptide oligonucleotide complex as described herein is a bladder cancer.

### Example 25

### Combination Treatment of Cancer

This example illustrates combination treatment of cancer using peptide nucleotide complexes of the present disclosure. The peptide and the oligonucleotide of a peptide nucleotide complex of the present disclosure are recombinantly expressed or chemically synthesized and then conjugated together with a linker and used directly. The peptide oligonucleotide complex is administrated in a pharmaceutical composition to a subject in need thereof as a therapeutic for cancer. The peptide within the peptide oligonucleotide complex is selected from any one of the peptides having a sequence of SEQ ID NO: 1-SEQ ID NO: 134 or SEQ ID NO: 306-SEQ ID NO: 335. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

One or more peptide oligonucleotide complexes are administered to a subject along with a treatment regimen of standard small molecule or chemotherapy, such as cisplatin, methotrexate, docetaxel, and etoposide, among others. The subject is a human or an animal. The pharmaceutical composition is administered intravenously, subcutaneously, intramuscularly, orally, intrathecally, intratumorally, intravitreally, or injected directly into the tumor microenvironment. The administered peptide oligonucleotide complex with standard small molecule therapy, treats a cancer condition in the subject. The cancer condition may be brain cancer, breast cancer, liver cancer, lung cancer, head and neck cancer, or colon cancer.

### Example 26

Imaging a Brain Condition with a TfR-Binding Peptide Oligonucleotide Complex

This example illustrates the imaging of a brain condition using a TfR-binding peptide oligonucleotide complex disclosed herein to transport detectable agent molecules attached to the peptides into the CNS. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

A peptide oligonucleotide complex of the present disclosure is conjugated to a detectable agent as described in EXAMPLE 20. The peptide detectable agent conjugate is administered to a subject in need thereof. The subject has a condition, such as brain cancer or Alzheimer's disease. The subject is a human or an animal. The peptide detectable agent conjugate is administered intravenously, subcutaneously, intramuscularly, orally, or injected directly into the tumor microenvironment.

The peptide is, optionally, further linked to an affinity moiety, for example, an affinity moiety such as F-18 for amyloid plaques. The peptide detectable agent crosses the BBB via TfR-peptide mediated transcytosis. Non-invasive imaging is performed to visualize the detectable agent in the brain, allowing for diagnosis or tracking of the condition in a subject.

### Example 27

Treatment of Brain Cancer with a TfR-Binding Peptide Oligonucleotide Complex

This example illustrates the treatment of a brain cancer using a TfR-binding peptide oligonucleotide complex as described herein to transport the nucleotide attached to the peptide into the CNS and into tumor cells. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18.

Optionally, peptide of the present disclosure is linked to an additional active agent, such as temozolomide, as described in EXAMPLE 21. The subject is a human or an animal. The peptide oligonucleotide complex is administered intravenously, subcutaneously, intramuscularly, orally, intrathecally, intravitreally, or injected directly into the tumor microenvironment. The peptide active agent conjugate is administered to a subject in need thereof. The subject has a brain cancer. Upon administration, the peptide oligonucleotide complex crosses the blood brain barrier (BBB) and accesses the brain parenchyma where the cancer is localized. The peptide active agent conjugate ameliorates and/or eradicates the brain cancer.

This data demonstrates that active agents that are generally prevented from accessing the brain (e.g., nucleotides are generally excluded by the BBB, temozolomide that is a substrate of P-glycoprotein transporters that prevent crossing of endothelial cells of the BBB) can be transported across the BBB efficiently to prevent and/or treat diseases and conditions (e.g., cancers) located in the brain.

### Example 28

### Cell-Penetrating Peptide Oligonucleotide Complex Fusions

This example describes peptide fusions with additional cell penetrating peptides. A TfR-binding peptide oligonucleotide complex of the present disclosure is chemically conjugated or recombinantly expressed as a fusion to an additional cell penetrating peptide moiety. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18. The additional cell penetrating peptide moiety is one or multiple Arg residues, such as an RRRRRRRR (SEQ ID NO: 143) sequence conjugated to, linked to, or fused at the N-terminus or C-terminus, or a Tat peptide with the sequence YGRKKRRQRRR (SEQ ID NO: 168) that is conjugated to, linked to, or fused to the N-terminus or C-terminus of any TfR-binding peptide of the present disclosure. Alternatively, the additional cell penetrating peptide moiety is selected from maurocaline, imperatoxin, hadrucalcin, hemicalcin, oplicalin-1, opicalcin-2, midkine (62-104), MCoTI-II, or chlorotoxin, which is fused to the N-terminus or C-terminus of any TfR-binding peptide of the present disclosure. Alternatively, the additional cell penetrating peptide moiety is selected from TAT such as CysTAT (SEQ ID NO: 141), S19-TAT (SEQ ID NO: 142), R8 (SEQ ID NO: 143), pAntp (SEQ ID NO: 144), Pas-TAT (SEQ ID NO: 145), Pas-R8 (SEQ ID NO: 146), Pas-FHV (SEQ ID NO: 147), Pas-pAntP (SEQ ID NO: 148), F2R4 (SEQ ID NO: 149), B55 (SEQ ID NO: 150), azurin (SEQ ID NO: 151), IMT-P8 (SEQ ID NO: 152), BR2 (SEQ ID NO: 153), OMOTAG1 (SEQ ID NO: 154), OMOTAG2 (SEQ ID NO: 155), pVEC (SEQ ID NO: 156), SynB3 (SEQ ID NO: 157), DPV1047 (SEQ ID NO: 158), C105Y (SEQ ID NO: 159), transportan (SEQ ID NO: 160), MTS (SEQ ID NO: 161), hLF (SEQ ID NO: 162), PFVYLI (SEQ ID NO: 163), or yBBR (SEQ ID NO: 164), which is fused to the N-terminus or C-terminus of any TfR-binding peptide of the present disclosure. Alternatively, the additional cell penetrating peptide moiety is fused to the N-terminus or C-terminus of any TfR-binding peptide of the present disclosure by a linker. The linker is selected from GGGSGGGSGGGS (SEQ ID NO: 243), KKYKPYVPVTTN (SEQ ID NO: 244) (linker from DkTx), or EPKSSDKTHT (SEQ ID NO: 245) (linker from human IgG3), or any other linker. Alternatively, the TfR-binding peptide, the additional cell penetrating peptide moiety, and, optionally, the linker are joined by other means. For example, the other means includes, but is not limited to, chemical conjugation at any location, fusion of the additional cell penetrating peptide moiety and/or the linker to the C-terminus of the TfR-binding peptide, co-formulation with liposomes, or other methods.

Cell-penetrating peptide fusions or conjugates are administered to a subject in need thereof. The subject is a human or animal and has a disease, such as a brain cancer or other brain condition. Upon administration, TfR-binding peptides promote transcytosis across the BBB and the additional cell-penetrating peptides promote crossing the cellular membranes to access intracellular compartments. Alternatively, upon administration, the TfR-binding peptides promote endocytosis into cells expressing TfR and the TfR-binding peptides and/or the the additional cell penetrating peptides promote release of the oligonucleotide into the cytoplasm or other subcellular compartments.

### Example 29

### Peptide Oligonucleotide Complexes to Promote Nuclear Localization

This example describes peptide complexes to promote nuclear localization. The peptide and the oligonucleotide of a TfR-binding peptide oligonucleotide complex of the present disclosure are recombinantly expressed or chemically synthesized and then conjugated together with a linker. The peptide within the peptide oligonucleotide complex is selected from any sequence of SEQ ID NO: 1-SEQ ID NO: 134 or SEQ ID NO: 306-SEQ ID NO: 335. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6, or to any of SEQ ID NO: 395-SEQ ID NO: 428 provided in TABLE 3, or to any genomic or ORF sequence provided in TABLE 18. The peptide oligonucleotide complexes are conjugated to, linked to, or fused to a nuclear localization signal, such as a four-residue sequence of K-K/R-X-K/R (SEQ ID NO: 299), wherein X can be any amino acid, or a variant thereof (Lange et al, J Biol Chem. 2007 Feb. 23; 282(8):5101-5). The complexes are administered to a subject in need thereof. The subject is a human or animal and has a disease, such as a brain cancer or other brain condition. Upon administration, TfR-binding peptides promote transcytosis across the BBB and the nuclear localization signal promotes trafficking to the nucleus and or the TfR-binding peptides promote endocytosis into cells expressing TfR.

### Example 30

Treatment of Brain Cancer with a Peptide Oligonucleotide Complex Comprising an Immunotherapy Agent

This example illustrates treatment of brain cancer (e.g., a glioblastoma, an astrocytoma, a midline glioma, a DIPG, a medulloblastoma, a MYC- or MYCN-amplified brain tumor, or an ependymoma) using a peptide oligonucleotide complex of the present disclosure. Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are comprise or are conjugated to, linked to, or fused to one or more target binding agents capable of binding a target molecule that comprises or encodes a protein such as a CTLA-4, a PD-1, or a PDL-1, or IL15, or a fused IL15/IL15Ra, IFNgamma, or anti-CD3 fusion polypeptides. In this experiment, the immunotherapeutic agent is a target binding agent capable of binding a target molecule of the PD-L1 RNA.

A TfR-binding peptide in the peptide within the peptide oligonucleotide complex having a sequence of any one of SEQ ID NO: 1-SEQ ID NO: 134 or SEQ ID NO: 306-SEQ ID NO: 335 is expressed recombinantly or chemically synthesized. The peptide is conjugated, linked, or complexed with an oligonucleotide that targets PD-L1 RNA. The conjugate is purified and formulated for injection into a subject (e.g., a mouse or a human).

Following administration to a subject in need thereof, the peptide construct comprising a TfR-binding peptide oligonucleotide complex targeting PD-L1 or a functional fragment thereof, penetrates the BBB via vesicular transcytosis and accumulates in the CNS and/or is taken up into a cell expressing TfR by endocytosis. Nuclear imaging (e.g., positron emission tomography, computed tomography (PET), or magnetic resonance imaging) and tissue or biopsy samples taken from the subject suffering from the brain cancer show that the peptide construct significantly reduces tumor burden in the subject. Optionally, whole body imaging (e.g., WBA or PET) shows that the peptide construct not only reduced the tumor size and sites of disease in the brain, but also at other location within the body of the subject (e.g., liver, lungs, and bone).

### Example 31

### Ibuprofen Peptide Oligonucleotide Complex

This example describes the conjugation of ibuprofen to a TfR-binding peptide oligonucleotide complex as described herein using a PEG linker. The peptide and the oligonucleotide of a TfR-binding peptide oligonucleotide complex are expressed recombinantly or can be chemically synthesized and then conjugated together with a linker. The peptide is optionally purified using liquid chromatography (e.g., reversed-phase, ion exchange, or size-exclusion chromatograph), or other known methods. A peptide oligonucleotide ibuprofen complex is produced using ibuprofen and a PEG linker, which forms an ester bond that can hydrolyze as described in “In vitro and in vivo study of poly(ethylene glycol) conjugated ibuprofen to extend the duration of action,” Scientia Pharmaceutica, 2011, 79:359-373, Nayak and Jain. Fischer esterification is used to conjugate ibuprofen with a short PEG, e.g., with triethylene glycol, to yield ibuprofen-ester-PEG-OH.

Following preparation of the PEG-ibuprofen conjugate as shown above, the hydroxyl moiety of PEG is activated with N,N′-disuccinimidyl carbonate (DSC) to form ibuprofen-ester-PEG-succinimidyl carbonate, which is then reacted with a lysine or the N-terminus of a peptide oligonucleotide complex to form an ibuprofen-ester-PEG-peptide oligonucleotide conjugate. The ibuprofen-peptide oligonucleotide complex is formulated for injection into a subject (e.g., a mouse or a human). Following administration of the conjugate, the peptide oligonucleotide complex penetrates the BBB via vesicular transcytosis and accumulates in the CNS. The conjugate can display anti-inflammatory activity, or free ibuprofen is released from the conjugate to provide anti-inflammatory activity. The free ibuprofen can result from hydrolysis that occurs after administration, such as hydrolysis at the ester bond.

The peptide oligonucleotide complex itself can modulate pain or it can be conjugated to an agent that modulates pain (e.g., ibuprofen). Such pain modulation may operate by various mechanisms such as modulating inflammation, autoimmune responses, direct or indirect action on pain receptors, cell killing, or programmed cell death. Ibuprofen-peptide oligonucleotide complexes are administered to a subject in need thereof. The subject can be a human or a non-human animal.

### Example 32

### Peptide Oligonucleotide Kᵥ1.3 Regulating Complex

This example describes a TfR-binding peptide oligonucleotide complex as described herein wherein the oligonucleotide modulates the expression of Kᵥ1.3 potassium channels for treatment of autoimmune diseases. In this case, the Kᵥ1.3 inhibitor is an oligonucleotide that can target a target molecule that comprises or encodes Kᵥ1.3 potassium channels. The peptide is complexed with a target binding agent capable of binding a target molecule that encodes a Kᵥ1.3 potassium channel. The target binding agent can be a nucleotide capable of binding an RNA encoding a Kᵥ1.3 potassium channel.

A peptide of the disclosure is expressed recombinantly or chemically synthesized, and then the N-terminus of the peptide is conjugated to, linked to, or fused to the oligonucleotide via an activated ester (e.g., NHS ester) in the presence of either DCC or EDC to produce a TfR-binding peptide-oligonucleotide conjugate.

The peptide oligonucleotide complex is administered to a subject in need thereof. The peptide construct is administered intravenously, subcutaneously, intramuscularly, intrathecally, intravitreally, or orally. The subject can be a human or a non-human animal. After administration, the peptide-Kᵥ1.3 inhibitor conjugate is delivered to the tissue or organ of interest via TfR-mediated transcytosis and/or TfR-mediated endocytosis. The peptide-dalazatide oligonucleotide complex ameliorates the autoimmune disease or condition the subject is being treated for, for example those caused by self-reactive T lymphocytes.

This data demonstrates that the TfR-binding oligonucleotide complexes of the present disclosure provide therapeutically effective concentrations of the Kᵥ1.3 inhibitor in the CNS or to other tissue to treat autoimmune disorders. The therapeutic activity is improved compared to treatment with the Kᵥ1.3 inhibitor alone.

### Example 33

### TfR-Binding Peptide Oligonucleotide Complex Kᵥ1.3 Inhibitor Conjugates for Treatment of a Neurodegenerative Disease

This example describes a TfR-binding peptide oligonucleotide complex that is an ion channel modulator. The ion channel modulator may be a target binding agent capable of binding a target molecule that is a molecule encoding a Kᵥ1.3 potassium. The ion channel modulator may be used for treatment of neurodegenerative diseases (e.g., Alzheimer's disease, Amyotrophic lateral sclerosis, Friedreich's ataxia, Huntington's disease, Lewy body disease, Parkinson's disease, Spinal muscular atrophy, Motor neuron disease, Lyme disease, Ataxia-telangiectasia, Autosomal dominant cerebellar ataxia, Batten disease, Corticobasal syndrome, Creutzfeldt-Jakob disease, Fragile X-associated tremor/ataxia syndrome, Kufor-Rakeb syndrome, Machado-Joseph disease, multiple sclerosis, chronic traumatic encephalopathy, or frontotemporal dementia).

A peptide of the disclosure is expressed recombinantly or chemically synthesized, and then the N-terminus of the peptide is conjugated to, linked to, or fused to one or ion channel modulators (e.g., a target binding agent capable of binding a target molecule that comprises a Kᵥ1.3 potassium channel via, for example, an activated ester (e.g., NHS ester) in the presence of either DCC or EDC to produce a TfR-binding peptide-anti-Kᵥ1.3 peptide oligonucleotide complex. The peptide oligonucleotide complex is administered to a subject in need thereof. The peptide oligonucleotide complex is administered intravenously, subcutaneously, intramuscularly, intrathecally, intravitreally, or orally. The subject can be a human or a non-human animal. After administration, the TfR-binding peptide-Kᵥ1.3 inhibitor peptide oligonucleotide complex is delivered to the tissue or organ of interest via TfR-mediated transcytosis and/or TfR-mediated endocytosis. The TfR-binding peptide-Kᵥ1.3 inhibitor peptide oligonucleotide complex penetrates the BBB via vesicular transcytosis and accumulates in the CNS. The TfR-binding oligonucleotide complex ameliorates the neurodegenerative disease or condition the subject is being treated for.

This demonstrates that the TfR-binding peptide oligonucleotide complexes of the present disclosure provide therapeutically effective level of Kᵥ1.3 inhibition to treat neurodegenerative diseases. The therapeutic activity is improved compared to treatment with the Kᵥ1.3 inhibitor alone.

### Example 34

### TfR-Binding Peptide Oligonucleotide BACE Regulating Complex

This example describes a TfR-binding peptide complexed with a nucleotide that is a BACE modulator, for treatment of Alzheimer's Disease. The peptide may be complexed with a nucleotide target binding agent capable of binding a target molecule that encodes BACE. The target molecule can be an RNA molecule encoding BACE.

A peptide and a nucleotide of a peptide oligonucleotide complex of the disclosure are expressed recombinantly or chemically synthesized, and then the N-terminus of the peptide, or any part of the peptide is conjugated to the nucleotide via, for example, an activated ester (e.g., NHS ester) in the presence of either DCC or EDC to produce a TfR-binding peptide oligonucleotide complex.

The peptide oligonucleotide complex construct is administered to a subject in need thereof. The peptide construct is administered intravenously, subcutaneously, intramuscularly, intrathecally, or orally. The subject can be a human or a non-human animal. After administration, the TfR-binding peptide-oligonucleotide complex is delivered to the tissue or organ of interest via TfR-mediated transcytosis and/or TfR-mediated endocytosis. The TfR-binding peptide-oligonucleotide complex is efficiently transported across the BBB via TfR-mediated vesicular transcytosis and accumulates in the CNS. The TfR-binding peptide-oligonucleotide complex ameliorates the Alzheimer's Disease in the subject.

This demonstrates that the TfR-binding peptide oligonucleotide complexes of the present disclosure provide therapeutically effective concentration of the BACE inhibitor to treat Alzheimer's Disease. The therapeutic activity is improved compared to treatment with the BACE inhibitor alone.

### Example 35

Treatment of Brain Cancer with a Peptide Oligonucleotide Complex Construct Comprising a Non-BBB Penetrating Active Agent

This example illustrates the treatment of a brain cancer using a TfR-binding peptide oligonucleotide complex comprising a TfR-binding peptide as described herein (e.g., a peptide having a sequence of any one of SEQ ID NO: 1-SEQ ID NO: 134 or SEQ ID NO: 306-SEQ ID NO: 335) conjugated to, linked to, or fused to a non-BBB penetrating active agent (e.g., a nucleotide target binding agent or an additional active agent such as doxorubicin) to transport that agent into the CNS via TfR-mediated transcytosis to treat a brain tumor (e.g., glioblastoma, astrocytoma, midline glioma, DIPG, medulloblastoma, MYC- or MYCN-amplified brain tumors, or ependymoma) that would otherwise not be treatable or only show very limited efficacy with the active agent alone. The nucleotide active agent may be a nucleotide target binding agent capable of binding a target molecule that is upregulated in cancer, such as a molecule encoding Myc, Myb, Fos, Jun, or NfKB.

A peptide oligonucleotide complex of the disclosure is constructed. The peptide may be expressed recombinantly or chemically synthesized. The peptide of the present disclosure is conjugated to, linked to, or fused to the oligonucleotide active agent as described in EXAMPLE 5 and EXAMPLE 6 and optionally to an additional active agent as described in EXAMPLE 21. The peptide oligonucleotide complex construct is administered intravenously, subcutaneously, intramuscularly, orally, intrathecally, intravitreally, or injected directly into a tumor microenvironment. The peptide construct is administered to a subject in need thereof. The subject is a human or an animal. The subject has a brain cancer. Upon administration, the peptide oligonucleotide complex crosses the blood brain barrier (BBB) and accesses the region(s) of the brain where the cancer is localized. The peptide oligonucleotide complex comprising the non-BBB penetrating agent ameliorates and/or eradicates the brain cancer.

This data demonstrates that the TfR-binding peptide oligonucleotide complexes of the present disclosure can be used to provide therapeutically effective concentrations of therapeutic agents (e.g., nucleotide target binding agents or additional active agents such as doxorubicin) in cells, tissues, or organs that may otherwise not be accessible for the therapeutic agents alone.

### Example 36

Targeting Bone Marrow using TfR-binding Peptide Oligonucleotide Complex

This example describes targeting bone marrow using a TfR-binding peptide oligonucleotide complex described herein. Optionally, the TfR-binding peptide oligonucleotide complexes can be conjugated to, linked to, or fused to an additional active or detectable agent for treatment or diagnosis of disease, respectively. The nucleotide active agent may be a nucleotide target binding agent capable of binding a target molecule that is upregulated in cancer, such as a molecule encoding Myc, Myb, Fos, Jun, or NfKB. The nucleotide may target gene expression in bone marrow.

The TfR-binding peptide oligonucleotide complex of the disclosure is constructed. The peptide may be expressed recombinantly or chemically synthesized. The TfR-binding peptide oligonucleotide complex is administered to a subject in need thereof. The TfR-binding peptide oligonucleotide complex is administered intravenously, subcutaneously, intramuscularly, orally, intrathecally, intravitreally, or injected directly into a tumor microenvironment. The subject is a human or an animal. After administration, the TfR-binding peptide oligonucleotide complex is delivered to the bone marrow via TfR-mediated transcytosis and/or TfR-mediated endocytosis.

The presence of the TfR-binding peptide oligonucleotide complex in the bone marrow of the subject is confirmed using tissue samples and/or non-invasive imaging, demonstrating that the TfR-binding peptide oligonucleotide complexes of the present disclosure successfully target TfR-expressing cells, tissues, or organs such as the bone marrow as an important tissue for therapeutic intervention.

### Example 37

### Targeting Erythroid Cells Using TfR-Binding Peptide Oligonucleotide Complex

This example describes targeting of erythroid cells or erythroid precursor cells using a TfR-binding peptide oligonucleotide complex as described herein. The TfR-binding peptide oligonucleotide complexes can optionally be conjugated to, linked to, or fused to an additional active or detectable agent. The peptide oligonucleotide complex can be used for treatment or diagnosis of disease. The nucleotide may be a target binding agent capable of binding a target molecule that is upregulated in cancer, such as a molecule encoding NUP98-KDM5A, NTRK1, JAK2, K-/N-RAS, HDAC, MDM2, LSD1, or CALR.

The TfR-binding peptide oligonucleotide complexes of the disclosure are constructed. The peptide may be expressed recombinantly or chemically synthesized. The peptide oligonucleotide complex is administered to a subject in need thereof. The peptide oligonucleotide complex is administered intravenously, subcutaneously, intramuscularly, intrathecally, intravitreally, or orally. The subject is a human or an animal. After administration, the TfR-binding peptide oligonucleotide complex is delivered to the erythroid cells or erythroid precursor cells via TfR-mediated transcytosis and/or TfR-mediated endocytosis.

The presence of the TfR-binding peptide oligonucleotide complex in the erythroid cells or erythroid precursor cells of the subject is confirmed using tissue (e.g., bone marrow samples) or blood samples or via non-invasive imaging, demonstrating that the TfR-binding peptide oligonucleotide complexes of the present disclosure successfully target TfR-expressing cells, tissues, or organs such as erythroid cells or erythroid precursor cells.

### Example 38

### Targeting Muscle Cells Using TfR-Binding Peptide Oligonucleotide Complex

This example describes targeting of muscle cells using a TfR-binding peptide oligonucleotide complex as described herein. The TfR-binding peptide oligonucleotide complexes can optionally be conjugated to, linked to, or fused to an additional active or detectable agent. The peptide oligonucleotide complex can be used for treatment or diagnosis of disease. The nucleotide may be a target binding agent capable of binding a target molecule that regulates muscle growth, such as a molecule encoding myostatin.

The TfR-binding peptide oligonucleotide complex of the disclosure is constructed. The peptide may be expressed recombinantly or chemically synthesized. The TfR-binding peptide oligonucleotide complex is administered to a subject in need thereof. The TfR-binding peptide oligonucleotide complex is administered intravenously, subcutaneously, intramuscularly, intrathecally, intravitreally, or orally. After administration, the TfR-binding peptide oligonucleotide complex is delivered to muscle cells or muscle tissue via TfR-mediated transcytosis and/or TfR-mediated endocytosis.

The presence of the TfR-binding peptide oligonucleotide complex in the muscle cells or muscle tissue (particularly the uptake and retention in skeletal muscle tissue) of the subject is confirmed using tissue (e.g., muscle or skeletal muscle tissue samples) or blood samples or via non-invasive imaging, demonstrating that the TfR-binding peptide oligonucleotide complexes of the present disclosure successfully target TfR-expressing cells, tissues, or organs such as muscle cells or muscle tissue. Optionally, growth of muscle is increased, or atrophy of muscle is reduced in the subject.

### Example 39

### Treatment of an Inflammatory Bowel Disease Using TfR-Binding Peptide Oligonucleotide Complex

This example describes treatment of an inflammatory bowel disease using a TfR-binding peptide oligonucleotide complex as described herein. Optionally, the TfR-binding peptide oligonucleotide complex is conjugated to, linked to, or fused to an additional active or detectable agent. The peptide oligonucleotide complex can be used for treatment or diagnosis of disease. The oligonucleotide may be a target binding agent capable of binding a target molecule that is pro-inflammatory in IBD, such as a molecule encoding TNF-α, ICAM-1, NF-κB, Smad7, CHST15, IL-12, IL-23, or IL-17.

The TfR-binding peptide oligonucleotide complex of the disclosure is constructed. The peptide may be expressed recombinantly or chemically synthesized. The TfR-binding peptide construct is administered to a subject in need thereof. The TfR-binding peptide oligonucleotide complex is administered intravenously, subcutaneously, intramuscularly, by suppository, or orally. The subject is a human or an animal. After administration, the TfR-binding peptide oligonucleotide complex is delivered to the intestines and accumulates in the glandular cells of the intestines.

The peptide oligonucleotide complex ameliorates and/or eradicates the inflammatory bowel disease. This demonstrates that the TfR-binding peptides of the present disclosure can be used in peptide oligonucleotide complexes to provide therapeutically effective concentrations within the intestinal tract for treatment of various diseases such as an inflammatory bowel disease.

### Example 40

TfR-Binding Peptide Oligonucleotide Complex for pH-Dependent Endosomal Delivery

This example describes development and in vitro testing of TfR-binding peptide oligonucleotide complexes capable of pH-dependent dissociation from TfR, for example, at endosomal pH (e.g., pH 5.4).

One or more additional histidine residues are introduced into the sequence of TfR-binding peptides within the peptide oligonucleotide complex (e.g., any one of SEQ ID NO: 1-SEQ ID NO: 134 or SEQ ID NO: 306-SEQ ID NO: 335) complexed with an oligonucleotide. The resulting histidine-enriched TfR-binding peptide oligonucleotide complexes are evaluated for their TfR binding in comparative binding experiments at various pH levels or ranges. Peptides with high TfR binding affinity at physiological pH but a significantly reduced binding affinity at lower pH levels such as endosomal pH of 5.4 are selected for cellular binding, uptake, and intra-endosomal or intra-vesicular release experiments.

TfR-binding peptide oligonucleotide complexes with high endosomal delivery capabilities are identified and characterized. These results demonstrate that the TfR-binding peptide oligonucleotide complexes of the present disclosure can exhibit, or can be modified to exhibit pH-dependent TfR binding kinetics that allows intra-vesicular release of TfR-binding peptide oligonucleotide complexes and TfR-binding peptide oligonucleotide complex comprising one or more active agents for endosomal and/or intracellular delivery. Higher levels of the peptide oligonucleotide complex may be delivered to or accumulate in the endosome due to dissociation from TfR prior to TfR recycling back to the cell surface.

In order to improve the intracellular delivery functions, the TfR-binding peptide oligonucleotide complexes as described herein are optionally modified to comprise a motif that facilitates low-pH endosomal release or escape of the peptide oligonucleotide complex or are constructed with a cleavable linker.

Cellular uptake and release experiments demonstrate that the TfR-binding peptide oligonucleotide complexes that comprise a motif for low-pH endosomal escape show are present in the cytosol at higher concentrations compared to peptides that do not comprise the motif for low-pH endosomal escape. This data demonstrates that the TfR-binding oligonucleotide complexes of the present disclosure can be successfully modified for enhanced intra-vesicular and intra-cellular delivery, including to subcellular compartments, while retaining their TfR binding capabilities. These peptide oligonucleotide complexes can optionally be used in combination with various therapeutic and/or compounds for treatment and/or diagnosis of diseases and conditions.

### Example 41

### TfR-Binding Peptide Oligonucleotide Complex for Treatment of a Disease Located in Non-CNS Tissue

This example describes treatment of a disease that is located in a non-CNS tissue that expresses TfR using the TfR-binding peptide oligonucleotide complexes described herein.

The peptide and the oligonucleotide of the TfR-binding peptide oligonucleotide complex of the disclosure are expressed recombinantly or chemically synthesized and then conjugated together with a linker. Optionally, an additional active agent used to treat the disease or condition can be conjugated to, linked to, or fused to the TfR-binding oligonucleotide complex. The active agent is, optionally, any NT disclosed herein (e.g., neurotensin (SEQ ID NO: 341) or a neurotensin variant (e.g., any one of SEQ ID NO: 350-SEQ ID NO: 356)), or a functional fragment thereof, fused to a TfR-binding peptide (e.g., any one of SEQ ID NO: 1-SEQ ID NO: 134 or SEQ ID NO: 306-SEQ ID NO: 335). The peptide-NT fusions may be any one of SEQ ID NO: 135-SEQ ID NO: 140. The TfR-binding peptide oligonucleotide complex is administered to a subject in need thereof. The TfR-binding peptide oligonucleotide complex is administered intravenously, subcutaneously, intramuscularly, intrathecally, intravitreally, intratumorally, intravesicular to the bladder, by suppository, or orally. The subject is a human or an animal. After administration, the TfR-binding peptide oligonucleotide complex accumulates in the non-CNS tissue that expresses TfR. The TfR-binding peptide oligonucleotide complex ameliorates the disease or condition.

Optionally the peptide oligonucleotide complex is administered directly to the eye, such as by intravitreal injection. The peptide nucleotide complex is endocytosed by TfR-expressing cells present and reaches therapeutic concentrations in the necessary cells of the eyes. Optionally the safety of the treatment is improved because lower concentrations are present in other tissues due to direct injection into the eye. The TfR-binding nucleotide complex ameliorates the disease or condition of the eye.

This data demonstrates that the TfR-binding peptide oligonucleotide complexes of the present disclosure effectively accumulate in TfR-expressing non-CNS tissue, and thus can be used to treat and/or prevent a disease or condition located in one or more of these tissues.

### Example 42

### TfR-Binding Peptide Nucleotide Complex for Treatment of a Disease Located in a CNS Tissue

This example describes treatment of a disease that is located in a CNS tissue using the TfR-binding peptide nucleotide complexes described herein.

The peptide and the oligonucleotide of the TfR-binding peptide nucleotide complex of the disclosure are expressed recombinantly or chemically synthesized and then conjugated together with a linker. Optionally, an additional active agent used to treat the disease or condition can be conjugated to, linked to, or fused to the TfR-binding nucleotide complex. The active agent is, optionally, any NT disclosed herein (e.g., neurotensin (SEQ ID NO: 341) or a neurotensin variant (e.g., any one of SEQ ID NO: 350-SEQ ID NO: 356)), or a functional fragment thereof, fused to a TfR-binding peptide (e.g., any one of SEQ ID NO: 1-SEQ ID NO: 134 or SEQ ID NO: 306-SEQ ID NO: 335). The peptide-NT fusions may be any one of SEQ ID NO: 135-SEQ ID NO: 140. The TfR-binding peptide nucleotide complex is administered to a subject in need thereof. The TfR-binding peptide nucleotide complex is administered intravenously, subcutaneously, intramuscularly, intrathecally, intravitreally, intratumorally, or orally. The subject is a human or an animal. After administration, the TfR-binding peptide nucleotide complex accumulates in the CNS tissue. The TfR-binding nucleotide complex ameliorates the disease or condition.

This data demonstrates that the TfR-binding peptide nucleotide complexes of the present disclosure effectively accumulate in CNS tissue, and thus can be used to treat and/or prevent a disease or condition located in one or more of these tissues.

### Example 43

### TfR-Binding Peptide Oligonucleotide Complex for Treatment of a Peripheral Cancer

This example describes treatment of a peripheral (e.g., non-CNS) cancer that expresses and/or overexpresses TfR using a TfR-binding peptide oligonucleotide complex described herein. The peripheral cancer can be breast cancer, liver cancer, lung cancer, colon cancer, brain cancer, spleen cancer, cancers of the salivary gland, kidney cancer, muscle cancers, bone marrow cell cancers, or skin cancer, genitourinary cancer, osteosarcoma, muscle-derived sarcoma, melanoma, head and neck cancer, a neuroblastoma, a CMYC-overexpressing tumor. The nucleotide may be a target binding agent capable of binding a target molecule that is upregulated peripheral cells in cancer, such as a molecule encoding IGF-1, androgen receptor, EGFR, ERBB3, Her2, GRB2, KRAS, MYC, YAP1, heat shock proteins, HIF1A, HIF1A, miR-21, MDM2, BCL2, FOXP3, DNMT1, HDACs, Myb, Fos, Jun, or NF-κB.

The TfR-binding peptide oligonucleotide complex of the disclosure is expressed recombinantly or chemically synthesized, by producing the peptide and producing the oligonucleotide, and then conjugate them together. Optionally, an additional active agent used to treat the peripheral cancer can be conjugated to, linked to, or fused to the TfR-binding peptide oligonucleotide complex to form a TfR-binding peptide oligonucleotide complex comprising an additional active agent. The TfR-binding peptide oligonucleotide complex is administered to a subject in need thereof. The TfR-binding peptide oligonucleotide complex is administered intravenously, subcutaneously, intramuscularly, orally, intrathecally, intravitreally, or intratumorally. The TfR-binding peptide oligonucleotide complex may be administrated intravitreally for melanoma in the eye. The subject is a human or an animal. After administration, the TfR-binding peptide oligonucleotide complex accumulates in the peripheral tumor tissue that expresses and/or overexpresses TfR. The TfR-binding peptide oligonucleotide complex ameliorates the peripheral cancer.

This data demonstrates that the TfR-binding peptide oligonucleotide complexes of the present disclosure effectively accumulate in TfR-expressing and/or TfR-overexpress peripheral cancers, and thus can be used to treat and/or prevent a cancer located in one or more of these tissues.

### Example 44

### TfR-Binding Peptide Oligonucleotide Complex for Treatment of a Spleen Cancer

This example describes treatment of a spleen cancer that expresses and/or overexpresses TfR using a TfR-binding peptide oligonucleotide complex described herein.

The TfR-binding peptide construct of the disclosure is expressed recombinantly or chemically synthesized, by producing the peptide and producing the oligonucleotide, and then conjugate them together. Optionally, an additional active agent active agent used to treat the spleen cancer can be conjugated to, linked to, or fused to the TfR-binding peptide oligonucleotide complex to form a TfR-binding peptide oligonucleotide complex with an additional active agent. The TfR-binding peptide oligonucleotide complex is administered to a subject in need thereof. The TfR-binding peptide oligonucleotide complex is administered intravenously, subcutaneously, intramuscularly, orally, intrathecally, intravitreally, or intratumorally. The subject is a human or an animal. After administration, the TfR-binding peptide oligonucleotide complex accumulates in the spleen cancer as demonstrated by molecular imaging (e.g., CT, MRI) and/or the analysis of tissue sample. The TfR-binding peptide oligonucleotide complex ameliorates the spleen cancer.

This data demonstrates that the TfR-binding peptide oligonucleotide complexes of the present disclosure effectively accumulate in TfR-expressing and/or TfR-overexpress cancers, such as spleen cancer, and thus can be used to treat and/or prevent a spleen cancer or other peripheral cancers.

### Example 45

### TfR-Binding Peptide Oligonucleotide Complex for Treatment of a Bone Marrow Cancer

This example describes treatment of a cancer located in the bone marrow and that expresses and/or overexpresses TfR using a TfR-binding peptide oligonucleotide complex described herein.

The TfR-binding peptide oligonucleotide complex of the disclosure is expressed recombinantly or chemically synthesized, by producing the peptide and producing the oligonucleotide, and then conjugate them together. Optionally, an additional active agent used to treat the bone marrow cancer can be conjugated to, linked to, or fused to the TfR-binding peptide oligonucleotide complex to form a TfR-binding peptide oligonucleotide complex with an additional active agent. The TfR-binding peptide oligonucleotide complex is administered to a subject in need thereof. The TfR-binding peptide oligonucleotide complex is administered intravenously, subcutaneously, intramuscularly, orally, intrathecally, intravitreally, or intratumorally. The subject is a human or an animal. After administration, the TfR-binding peptide oligonucleotide complex accumulates in the bone marrow cancer as demonstrated by molecular imaging (e.g., CT, MRI) and/or the analysis of tissue sample. The TfR-binding peptide oligonucleotide complex ameliorates the bone marrow cancer.

This data demonstrates that the TfR-binding peptide oligonucleotide complexes of the present disclosure effectively accumulate in TfR-expressing and/or TfR-overexpress peripheral cancers, such as cancers of or located in the bone marrow, and thus can be used to treat and/or prevent a bone marrow cancer or other peripheral cancers.

### Example 46

### Neurotensin Comprising Peptide Oligonucleotide Complex, Delivery to CNS, and Activation of Neuronal CRE Reporter Mice

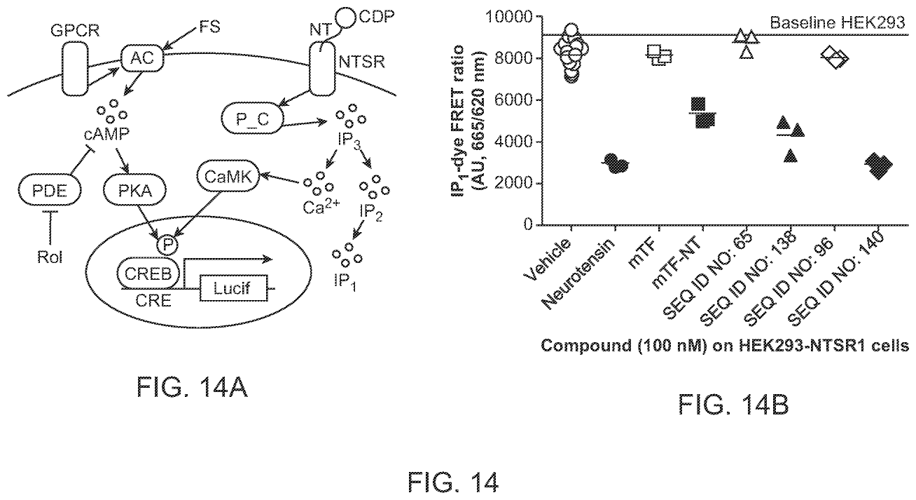

This example describes activation of neuronal CRE transporter mice using peptide constructs comprising one or more TfR-binding peptides as described herein. In this case, a fusion peptide comprising TfR-binding peptides and a neurotensin peptide was used. Peptides corresponding to SEQ ID NO: 65, SEQ ID NO: 66, and SEQ ID NO: 96 (SEQ ID NO: 65, SEQ ID NO: 66, and SEQ ID NO: 96 are SEQ ID NO: 1, SEQ ID NO: 2, and SEQ ID NO: 32, respectively, with an added N-terminal GS) were fused with neurotensin at the C-terminus of each peptide to produce the peptide-NT complexes SEQ ID NO: 138 (GSREGCASRCTKYNAELEKCEARVSSMSNTEETCVQELFDLLHCVDHCVSQGSSELYE NKPRRPYIL), SEQ ID NO: 139 (GSREGCASRCTKYNAELEKCEARVMSMSNTEEDCEQELEDLLHCLDHCHSQGSSELYE NKPRRPYIL), and SEQ ID NO: 140 (GSREGCASRCMKYNDELEKCEARMMSMSNTEEDCEQELEDLLYCLDHCHSQGSSELY ENKPRRPYIL), respectively (SEQ ID NO: 135 (REGCASRCTKYNAELEKCEARVSSMSNTEETCVQELFDLLHCVDHCVSQGSSELYENK PRRPYIL), SEQ ID NO: 136 (REGCASRCTKYNAELEKCEARVMSMSNTEEDCEQELEDLLHCLDHCHSQGSSELYEN KPRRPYIL), and SEQ ID NO: 137 (REGCASRCMKYNDELEKCEARMMSMSNTEEDCEQELEDLLYCLDHCHSQGSSELYEN KPRRPYIL) with N-terminal GS, respectively). The downstream activity of neurotensin involves intracellular Ca²⁺ regulation and cAMP response element (CRE) driven transcriptional programs (FIG. 14A), and its modulation has been explored for suppression of chronic pain. CDP-NT peptide constructs engage the neurotensin receptor were generated. SEQ ID NO: 138, SEQ ID NO: 139, and SEQ ID NO: 140 were expressed recombinantly in 293F cells and purified. Purified peptide-NT fusions (e.g., SEQ ID NO: 138-SEQ ID NO: 140) treated with DTT (a reducing agent) display a gel shift relative to the unreduced sample, suggesting that the recombinant NT peptides contain cystines. Molecular weights of the purified peptides were verified using mass spectrometry.

Binding to the neurotensin receptor was demonstrated with a HEK-293 cell line expressing NTSR1. To demonstrate that the neurotensin extension on various proteins was functional, NTSR activity in HEK293 cells, or HEK293 cells transduced with a lentivector delivering human NTSR1 (HEK293-NTSR1), was measured using the IP-One-Gq kit (CisBio 62IPAPEB, FIG. 14B). Cells were grown in DMEM+10% fetal bovine serum, removed from the plates with Accutase, pelleted, and suspended in Hanks Buffered Salt Solution at a density of 1.5×10⁶ cells per mL. HTFR reactions were set up in HTFR 96 well low volume plates (CisBio #66PL96025) according to the manufacturer's instructions. 10,000 cells (7 μL) were used per 25 μL reaction. The plate was incubated for 60 mins at 37° C. Then 3 μL IP1-d2 working solution was then added, followed by 3 μL Anti IP1-Cryptate working solution. After a 1 hour incubation at room temperature, the plate was scanned in a Perkin Elmer 2104 EnVision Multilabel Reader for fluorescence emission after excitement at 665 nm and 620 nm wavelengths. FRET ratio was calculated as 10,000×(Signal 665 nm/Signal 620 nm). In mammalian HEK-293 cells neurotensin (NT) receptor engagement showed IP1 accumulation only in response to NT or NT peptide constructs (SEQ ID NO: 138 and SEQ ID NO: 140, as well as mTf-NT and NT, but not for SEQ ID NO: 65 or SEQ ID NO: 96, vehicle, or mTf), N=3 wells for all except vehicle, which had N=36 (FIG. 14B). Horizontal bar indicates sample mean.

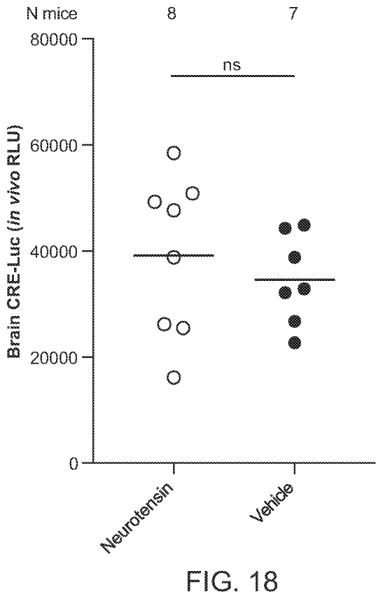

Delivery of NT to the CNS was demonstrated with transgenic mice expressing firefly luciferase under the control of a cyclic AMP response element (CRE). Under conditions of elevated cyclic AMP (cAMP) or other mechanisms that activate the transcription factor CRE Binding Protein 1 (CREB), cells in these mice express luciferase, which are measured in whole animal luminescence instrumentation (IVIS imager) after IV dosage with a luciferin formulated for animal use. Neurotensin receptor expression was limited to subsets of cells in the CNS, including the frontal cortex. Upon activation, a signal transduction pathway is activated that culminates in CREB phosphorylation and CRE-mediated transcription. The endogenous ligand of the neurotensin receptor, neurotensin, is a neuropeptide that cannot cross the BBB by itself (demonstrated in FIG. 18, mice dosed with 300 nmol NT or vehicle showed identical CRE-Luc-driven luminescence levels). Therefore, IV dosage of NT alone will not result in luciferase production in the brains of the CRE reporter mice. However, IV dosage of a fusion peptide comprising neurotensin and a TfR binding protein of the disclosure demonstrated BBB penetration as evinced by CRE-driven luciferase induction in the brains of the CRE reporter mice. All three of the of peptide-NT complexes (SEQ ID NO: 138, SEQ ID NO: 139, and SEQ ID NO: 140) tested exhibited delivery across the BBB into the brain, as determined by immunohistochemistry analysis, as well as functional engagement by NT on the NT receptor.

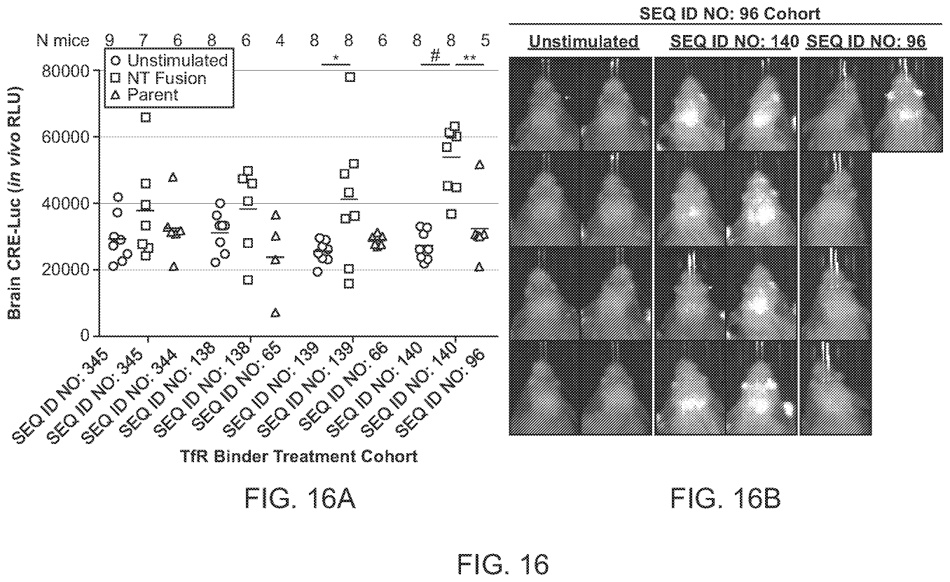

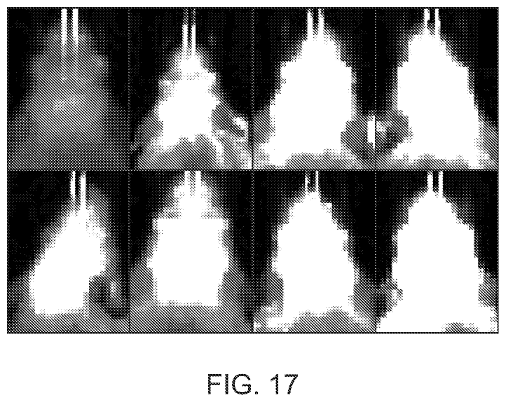

CRE-driven luciferase induction in vivo was verified by luciferin dosage four hours after administration of forskolin and rolipram (FIG. 17), potent CRE inducers via activation of adenylyl cyclase and inhibition of cAMP phosphodiesterase, respectively. CRE-luc GPCR reporter mice were administered 1.68 mg rolipram (Sigma Aldrich R6520) and 0.84 mg forskolin (Sigma Aldrich 344270) by intraperitoneal (IP) injection (200 μL in 17% DMSO solution), or TfR-binding peptide or other test and control peptides by intravenous tail vein injection; dosages were 100 nmol of TfR-binding peptides with or without NT (SEQ ID NO: 65, SEQ ID NO: 66, SEQ ID NO: 96, or SEQ ID NO: 138-SEQ ID NO: 140) or 12 nmol transferrin (SEQ ID NO: 344, MRLAVGALLVCAVLGLCLADYKDEHHHHHHGLNDIFEAQKIEWHEGGGSVPDKTVK WCAVSEHENTKCISFRDHMKTVLPPDGPRLACVKKTSYPDCIKAISASEADAMTLDGG WVYDAGLTPNNLKPVAAEFYGSVEHPQTYYYAVAVVKKGTDFQLNQLEGKKSCHTG LGRSAGWVIPIGLLFCKLSEPRSPLEKAVSSFFSGSCVPCADPVAFPKLCQLCPGCGCSST QPFFGYVGAFKCLKDGGGDVAFVKHTTIFEVLPEKADRDQYELLCLDNTRKPVDQYED CYLARIPSHAVVARKNNGKEDLIWEILKVAQEHFGKGKSKDFQLFSSPLGKDLLFKDSA FGLLRVPPRMDYRLYLGHNYVTAIRNQQEGVCPEGSIDNSPVKWCALSHLERTKCDEW SIISEGKIECESAETTEDCIEKIVNGEADAMTLDGGHAYIAGQCGLVPVMAEYYESSNCA IPSQQGIFPKGYYAVAVVKASDTSITWNNLKGKKSCHTGVDRTAGWNIPMGMLYNRIN HCKFDEFFSQGCAPGYEKNSTLCDLCIGPLKCAPNNKEEYNGYTGAFRCLVEKGDVAF VKHQTVLDNTEGKNPAEWAKNLKQEDFELLCPDGTRKPVKDFASCHLAQAPNHVVVS RKEKAARVKAVLTSQETLFGGSDCTGNFCLFKSTTKDLLFRDDTKCFVKLPEGTTPEKY LGAEYMQSVGNMRKCSTSRLLEACTFHKH) or transferrin-NT (SEQ ID NO: 345, MRLAVGALLVCAVLGLCLADYKDEHHHHHHGLNDIFEAQKIEWHEGGGSVPDKTVK WCAVSEHENTKCISFRDHMKTVLPPDGPRLACVKKTSYPDCIKAISASEADAMTLDGG WVYDAGLTPNNLKPVAAEFYGSVEHPQTYYYAVAVVKKGTDFQLNQLEGKKSCHTG LGRSAGWVIPIGLLFCKLSEPRSPLEKAVSSFFSGSCVPCADPVAFPKLCQLCPGCGCSST QPFFGYVGAFKCLKDGGGDVAFVKHTTIFEVLPEKADRDQYELLCLDNTRKPVDQYED CYLARIPSHAVVARKNNGKEDLIWEILKVAQEHFGKGKSKDFQLFSSPLGKDLLFKDSA FGLLRVPPRMDYRLYLGHNYVTAIRNQQEGVCPEGSIDNSPVKWCALSHLERTKCDEW SIISEGKIECESAETTEDCIEKIVNGEADAMTLDGGHAYIAGQCGLVPVMAEYYESSNCA IPSQQGIFPKGYYAVAVVKASDTSITWNNLKGKKSCHTGVDRTAGWNIPMGMLYNRIN HCKFDEFFSQGCAPGYEKNSTLCDLCIGPLKCAPNNKEEYNGYTGAFRCLVEKGDVAF VKHQTVLDNTEGKNPAEWAKNLKQEDFELLCPDGTRKPVKDFASCHLAQAPNHVVVS RKEKAARVKAVLTSQETLFGGSDCTGNFCLFKSTTKDLLFRDDTKCFVKLPEGTTPEKY LGAEYMQSVGNMRKCSTSRLLEACTFHKHGSSELYENKPRRPYIL) in PBS. 4 hours after administration, mice were administered 100 μL of 30 mg/mL D-Luciferin (RediJect D-Luciferin Ultra, PerkinElmer 770505) by IP injection and anesthetized by isoflurane exposure after 10 minutes of unrestricted activity. Luminescence was measured in anesthetized mice on a Xenogen IVIS instrument with a 1 min exposure. For dosing with rolipram and forskolin as positive controls, high levels of luminescence from the animal's brain are seen in the miced dosed (FIG. 17). For the test peptides for comparison, identical regions of interest encompassing the entire brain were draw and quantified using LivingImage software (version 4.0, PerkinElmer, FIG. 16). Mice were censored if their luminescence levels were >4 SD from the mean/SD luminescence of the other mice in the cohort, or if their luciferin injections failed to hit the peritoneal cavity. For the former, this was interpreted as an incidental physiological response unrelated to drug treatment or NT receptor modulation. For the latter, this was identified by failure of a fluorescent dye (pre-formulated in the RediJect D-Luciferin Ultra, read on the ICG filter set over 5 seconds) to distribute evenly throughout the animal's abdomen; this indicates insufficient perfusion and incomplete exposure of the whole mouse to luciferin, rendering the animal's brain luminescence quantitation questionable. Animals that were validated in this assay were given 1 week to return to steady state (low) luciferase expression and were then tested with neurotensin peptide or a fusion peptide comprising a TfR binding peptide fused to neurotensin.

Luminescence (via intraperitoneal luciferin dosage) was imaged either before (“Unstimulated”) or four hours after intravenous administration of parent TfR-binding peptides (SEQ ID NO: 65, SEQ ID NO: 66, SEQ ID NO: 96, or SEQ ID NO: 354) or CDP-NT peptide constructs (SEQ ID NO: 138-SEQ ID NO: 140), or the murine transferrin-NT construct (SEQ ID NO: 345), using matched cohorts (FIG. 16A). Transgenic Mice were first dosed with luciferin and luminescence was measured (“unstimulated”). Then the same mice were treated with either (i) the parent peptide (SEQ ID NO: 65, SEQ ID NO: 66, SEQ ID NO: 96, or SEQ ID NO: 354), and then dosed again with luciferin and luminescence was measured (“parent”), or (ii) the CDP-NT peptide constructs and murine transferrin-NT construct (SEQ ID NO: 345, SEQ ID NO: 138, SEQ ID NO: 139, SEQ ID NO: 140), and then dosed again with luciferin, and luminescence was measured (“NT fusion”). Quantitation is shown in FIG. 16A. Horizontal bar indicates sample mean. Significance was determined using a T-test (unpaired, 2-tailed). (*: P<0.05. **: P<0.01. #: P<0.0001.) Images for the SEQ ID NO: 96 cohort are show with pseudocolor luminescence in FIG. 16B. The mice had a measurable basal level of CRE activity driving luciferase expression in the brain (“unstimulated” readings), and this was not increased by dosing of the parent peptides (“parent” readings). However, the level of CRE activity was significantly elevated in mice administered the peptide fusions comprising TfR-binding peptides of this disclosure and NT, the CDP-NT peptide constructs of SEQ ID NO: 139 and SEQ ID NO: 140 (FIG. 16). The level of CRE activity was also elevated for mice treated with the CDP-NT peptide construct SEQ ID NO 138 and for the murine transferrin-NT fusion SEQ ID NO: 344, but was not statistically elevated as it was in the other CDP-NT peptide constructs (SEQ ID NO: 139 and SEQ ID NO: 140). Naked NT peptide alone, administered at 300 nmol (three times the dose of the peptide-NT fusion constructs), fails to activate the reporter in the brain after intravenous administration (FIG. 18), as expected as it does not cross the BBB. Altogether, this demonstrates not only CNS accumulation, but BBB penetration and neuronal access of a molecule fused to SEQ ID NO: 65 variants, as well as engagement of neurotensin receptor by NT fusions of peptides of this disclosure.

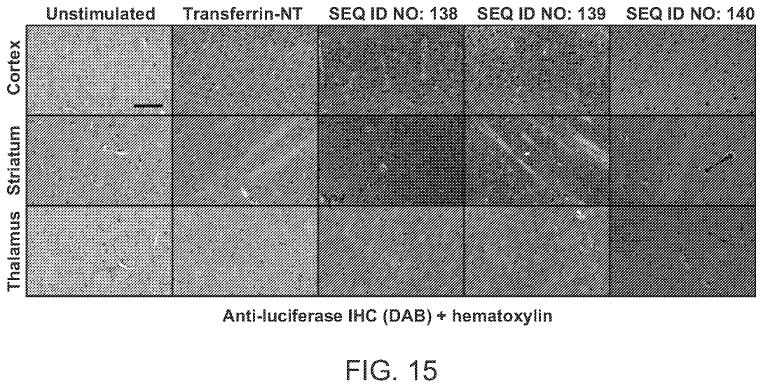

The ability of peptides to deliver NT to areas of the CNS was confirmed by immunohistochemistry (IHC). By IHC staining, luciferase levels were visibly higher in mice treated with neurotensin variants of SEQ ID NO: 65, SEQ ID NO: 66, and SEQ ID NO: 96 (e.g., SEQ ID NO: 138-SEQ ID NO: 140) (FIG. 15), particularly SEQ ID NO: 34 and SEQ ID NO: 35, whereas lower staining is visible in mice treated with Transferrin-NT (SEQ ID NO: 345) and much lower with mice that received no peptides (unstimulated). Formalin-fixed, paraffin-embedded brains were sectioned on a Microm HM 355S microtome at 6 μm and mounted on positively charged Superfrost Plus slides (Fisher 12-550-15). Staining was automated on a Ventana Discovery Ultra IHC/ISH autostainer using kits and reagents designed for use on this instrument: EZ Prep deparaffinization reagents (Ventana 950-100), Protease 3 antigen retrieval kit (Ventana 760-2020), anti-rabbit HQ (Ventana 760-4815), anti-HQ-HRP (Ventana 760-4815), ChromoMap DAB kit (Ventana 760-159), hematoxylin (Ventana 760-2021), and bluing reagent (Ventana 760-2037), all at manufacturer's default settings. The primary anti-luciferase antibody was from Abcam (ab21176, concentration 1:350) and was diluted with Ventana antibody diluent with casein (Ventana 760-219). The sequence was as follows. Sections were deparaffinized at 69° C., and then antigen retrieval took place at 35° C. for 4 mins. Slides were rinsed and warmed to 37° C. prior to primary antibody addition by hand (1:350). Slides were incubated with the primary antibody for 28 minutes and rinsed. Subsequent antibody additions (followed by rinsing) were anti-rabbit HQ and then anti-HQ HRP, incubating 16 mins apiece. Slides were then DAB treated and counterstained with hematoxylin and bluing reagent (8 minutes apiece). Images in FIG. 15 were mildly contrast-enhanced, with all image modifications performed identically across all images within a set without regard to treatment group. Immunohistochemistry showed enhanced luciferase expression in the cortex, striatum, and thalamus of mice 4 hours after CDP-NT administration. Scale bar (top left panel) is 100 μm (FIG. 15). All panels show the same magnification.

The peptide constructs of the present disclosure comprising a TfR binding peptide fused to neurotensin SEQ ID NO: 138-SEQ ID NO: 140 induced CRE-driven luciferase in the brains of the CRE reporter mice, demonstrating that the TfR binding peptides of the present disclosure are capable of transporting active agents (e.g., peptides such as neurotensin) efficiently across the BBB that are otherwise not able to access the brain. Other peptide constructs of the present disclosure comprising a TfR binding peptide fused to neurotensin may also induce CRE-driven luciferase in the brains of CRE reporter mice.

Any TfR-binding peptide disclosed herein can be used fused to neurotensin to induce CRE-driven luciferase in the brains of the CRE reporter mice, demonstrating that the TfR binding peptides of the present disclosure are capable of transporting active agents (e.g., peptides such as neurotensin) efficiently across the BBB that are otherwise not able to access the brain.

This data demonstrates that the TfR-binding peptides oligonucleotide complexes of the present disclosure of the present disclosure can provide therapeutically effective concentration of the NT receptor binding peptides in the CNS to effectively treat nociceptive or neuropathic pain. Any peptide oligonucleotide complex disclosed herein can be used to transport neurotensin (NT) into the CNS. The peptides can be complexed to NT to form a CDP-NT peptide construct via recombinant fusion, chemical conjugation, or by other means.

Any peptide oligonucleotide complexes of the present disclosure (e.g., including any one of oligonucleotide sequences provided in TABLE 7, EXAMPLE 50-EXAMPLE 54, or any one of SEQ ID NO: 364-SEQ ID NO: 394, linked or conjugated to SEQ ID NO: 1-SEQ ID NO: 134 or SEQ ID NO: 306-SEQ ID NO: 335) are described. Any peptide oligonucleotide complexes of the present disclosure can have oligonucleotides complementary to any target in TABLE 4, TABLE 5, TABLE 6 or to any of SEQ ID NO: 395-SEQ ID NO: 428 provided in TABLE 3, or to any genomic or ORF sequence provided in TABLE 18. These peptide oligonucleotide complexes can be used in transporting active agents (e.g., peptides such as neurotensin, or a target binding agent capable of binding a target molecule can target an upregulated cancer gene or RNA) efficiently across the BBB that are otherwise not able to access the brain.

### Example 47

Treatment or Management of Pain with a Peptide Oligonucleotide Complex-Active Agent Construct

This example describes a method for treating or managing pain. This method is used as a treatment for acute and/or chronic symptoms. A peptide oligonucleotide complex of the disclosure is constructed and administered in a pharmaceutical composition to a patient as a therapeutic for pain as a result of injury or other condition as described herein. The peptide oligonucleotide complex of the present disclosure comprises an oligonucleotide targeting agent that targets a gene encoding an ion channel, such as Naᵥ1.7. The peptide is expressed recombinantly or chemically synthesized, wherein the peptide within the peptide oligonucleotide complex is selected from a TfR-binding peptide as described herein (e.g., SEQ ID NO: 1-SEQ ID NO: 134 or SEQ ID NO: 306-SEQ ID NO: 335). In some aspects, the active agent is the oligonucleotide which acts to regulate gene or protein expression such as that of Naᵥ1.7 ion channel. In some aspects, an additional active agent is linked to, fused to, or complexed with the peptide oligonucleotide complex. In some aspects, the additional active agent is an NSAID pain reliever. In some aspects, the additional active agent is lidocaine. In some aspects, the additional active agent is a Naᵥ1.7 ion channel inhibitor. The peptide oligonucleotide complex is formulated for administration and administered to the subject. Following administration, the peptide oligonucleotide complex targets to the area, tissue, or system affected by pain. Such pain modulation may operate by various mechanisms such as modulating inflammation, autoimmune responses, direct or indirect action on pain receptors, cell killing, or programmed cell death. One or more peptide oligonucleotide complexes are administered to a human or animal subcutaneously, intravenously, intramuscularly, intrathecally, or orally, or are injected.

### Example 48

### Extension of Peptide Plasma Half-Life Using Serum Albumin-Binding Peptide Oligonucleotide Complex Constructs

This example demonstrates a method of extending the serum or plasma half-life of a peptide oligonucleotide complex using serum albumin-binding peptide oligonucleotide complexes as disclosed herein. A peptide, a nucleotide, a linker, or any other component within the peptide oligonucleotide complex is modified in order to increase its plasma half-life. The peptide nucleotide complex and the serum half-life extending moiety are fused recombinantly, chemically synthesized as a single fusion, separately recombinantly expressed and conjugated, or separately chemically synthesized and conjugated or otherwise constructed. Fusing the peptide to a serum albumin-binding peptide extends the serum half-life of the peptide oligonucleotide complex. The peptide oligonucleotide complex is, for example, conjugated or fused to a serum albumin-binding peptide, such as SA21 (SEQ ID NO: 357). Optionally, the peptide fused to SA21 is linked to SA21 via a peptide linker having a sequence of SEQ ID NO: 358. The linker having a sequence corresponding to SEQ ID NO: 358 links two separately functional CDPs to incorporate serum half-life extension function into the peptide or peptide construct. The linker having a sequence corresponding to SEQ ID NO: 358 enables SA21 to cyclize without steric impediment from either member of the peptide construct. Alternatively, conjugation of the peptide oligonucleotide complex to an albumin binder, such as Albu-tag or a fatty acid, such as a C₁₄-C₁₈ fatty acid, is used to extend plasma half-life. Plasma half-life is also optionally extended as a result of reduced immunogenicity by using minimal non-human protein sequences.

### Example 49

### Comparison of Dose Toxicity of a TfR-binding Peptide Oligonucleotide Complex to a TfR-binding Antibody Oligonucleotide Complex

This example describes the comparison of the dose toxicity of a TfR-binding peptide oligonucleotide complex of this disclosure to anti-TfR antibody oligonucleotide complex when administered to a murine subject. Optionally, the oligonucleotide targeting agent targets a gene that encodes for BACE. An anti-TfR antibody oligonucleotide complex is administered to a subject at doses of 5 mg/kg, 25 mg/kg or 50 mg/kg, corresponding to molar doses per 25 g mouse mass of about 0.84 nmol, 4.2 nmol, and 8.4 nmol, respectively, as described in Couch, et al, 2013 (Couch et al, Sci Transl Med. 2013 May 1; 5(183):183ra57). A TfR-binding peptide oligonucleotide complex of this disclosure is administered to a subject at doses of about 31 mg/kg, corresponding to a molar concentration of about 100 nmol per 25 g mouse mass. Alternatively, a TfR-binding peptide oligonucleotide complex of this disclosure is administered to a subject at doses of 0.84 nmol, 4.2 nmol and 8.4 nmol or 100 nmol. Subjects receiving 31 mg/kg, or about 100 nmol per 25 g mouse mass, of the TfR-binding peptide oligonucleotide complex show effective pharmacodynamic and pharmacokinetic properties without signs of distress or toxicity over the course of at least 24 hours. Meanwhile subjects receiving 5 mg/kg, or about 0.84 nmol per 25 g mouse mass, of the anti-TfR antibody oligonucleotide complex show reduced therapeutic efficacy, such as reduced pharmacodynamic amyloid beta inhibition, as compared to the subjects receiving 25 or 50 mg/kg, or 4.2 or 0.4 nmol per 25 kg mouse mass. The 25 or 50 mg/kg, or 4.2 or 0.4 nmol per 25 kg mouse mass, doses of the anti-TfR antibody induce lethargy, distress, and hemolysis, or reduced reticulocyte count or other toxicities, within at least 30 minutes of administration. The results demonstrate that the therapeutic window (the dosage above which a therapeutic pharmacodynamic response is seen but below which toxicity is observed) is wider for peptide-based therapeutics than for antibody-based therapeutics. The results further demonstrate that TfR-binding peptide oligonucleotide complex-based therapeutics show less off-target binding and lower immune response as compared to TfR-binding antibody-based therapeutics, due to the smaller protein lengths (approximately 50 amino acids) providing fewer epitopes for an adaptive immune response and smaller surface area.

### Example 50

TfR-Binding Peptide Oligonucleotide Complex Using siRNA for Treatment of IBD

This example describes treatment of inflammatory bowel (IBD) using a TfR-binding peptide nucleotide complex described herein. The receptor for IL-23, IL-23R (gene name IL23R), is a pro-inflammatory cytokine that recruits NK and T cells to gut mucosa. IL-23R is expressed in NK and T cells and mutations in IL23R polymorphisms are associated with hereditary ulcerative colitis. Anti-IL-23 antibodies have shown therapeutic utility in IBD. The nucleic acid portion of the peptide oligonucleotide complex comprises siRNA targeting the IL23R transcript. Short sequences in the IL-23R mRNA are identified (e.g., 21 nt sequences in the IL-23R mRNA), beginning with AA and ending in TT (or UU in RNA) that are between 30-60% G/C in content and complementary sequence to the IL-23R mRNA used in the complex. For example, any 21 mer complementary across the IL-23R mRNA that has imperfect complementarity (e.g., no more than 85% complementarity, or having at least 3 to 4 mismatches) or no to low complementarity (e.g., no more than 75%, 65%, 50%, or 30% complementarity) relative to other sequences in transcriptome (to reduce off target effects) may be used, with an optimal length that fits into RISC complex (e.g., a 21 mer+/−up to 5 nt).

The siRNA may bind a target molecule of SEQ ID NO: 396. Duplex structures (e.g., dsRNA) for modulating IL-23R mRNA can include: SEQ ID NO: 387-SEQ ID NO: 394 provided in TABLE 14 describes exemplary IL-23R siRNAs which are four siRNA pairs. It is understood to that within each pair of complimentary sequences described (e.g., SEQ ID NO: 387 and SEQ ID NO: 388, SEQ ID NO: 389 and SEQ ID NO: 390, etc.) are together part of the same complex and are partial reverse complements to one another.

TABLE 14
Examples of IL-23R siRNAs

| SEQ ID NO | Sequence |
|---|---|
| SEQ ID NO: 387 | 3′-AAGUGAUUCGUUGUGUUAUAA-5′ |
| SEQ ID NO: 388 | 5′-AAUUCACUAAGCAACACAAUA-3′ |
| SEQ ID NO: 389 | 3′-UAGAAGUGUACCUGUGUACAA-5′ |
| SEQ ID NO: 390 | 5′-AAAUCUUCACAUGGACACAUG-3′ |
| SEQ ID NO: 391 | 3′-CGACGGAACGUUAGACUUGAA-5′ |
| SEQ ID NO: 392 | 5′-AAGCUGCCUUGCAAUCUGAAC-3′ |
| SEQ ID NO: 393 | 3′-CCACCGUUCUUCAUGAACCAA-5′ |
| SEQ ID NO: 394 | 5′-AAGGUGGCAAGAAGUACUUGG-3′ |

Flanking ˜2-3 nucleotides are joined by phosphodiester (PO) or phosphorothioate (PS) linkages. All other backbones are PO linkages. Sugar chemistries are RNA, either regular (—OH) or 2′ modified (such as 2′-O-Me, 2′-F).

The TfR-binding peptide and the oligonucleotide of the peptide oligonucleotide complex are each expressed recombinantly or chemically synthesized and then conjugated together with a linker. Optionally, the linker is cleavable. Optionally, the TfR-binding peptide has reduced affinity for TfR at pH lower than 7.4. The nucleic acid portion of the peptide oligonucleotide complex is, targeted against any portion of the IL23R mRNA (NCBI Refseq ID NM_144701.3 IL23R [organism=Homo sapiens][GeneID=149233], SEQ ID NO: 396), or a functional fragment thereof. The TfR-binding peptide oligonucleotide complex is administered to a subject in need thereof. The TfR-binding peptide oligonucleotide complex is administered intravenously, subcutaneously, intramuscularly, by suppository, or orally. The subject is a human or an animal. Mouse model experiments may demonstrate pharmacodynamic response in immunodeficient mice that have been engrafted with human T cells and treated with gut microbes known to induce IBD in this model. After administration, the TfR-binding peptide oligonucleotide complex accumulates in tissue related to the disease, including peripheral or resident immune cells and the IL-23R mRNA is degraded. The TfR-binding peptide nucleotide complex ameliorates the IBD: Reduced symptoms of IBD are exhibited. In patients these symptoms may include diarrhea, fatigue, cramping, fever, rectal bleeding. In mice these symptoms may include weight loss, hunching, loose stools, scruffy coat, gut hyperplasia and/or goblet cell depletion, histopathological markers of inflammation).

This data demonstrates that the TfR-binding peptide oligonucleotide complexes of the present disclosure effectively treat IBD.

### Example 51

### TfR-Binding Peptide Oligonucleotide Complex Using a Gapmer for Treatment of CNS Disease

This example describes treatment of CNS disease (e.g., Alzheimer's, Frontotemporal Dementia, and chronic traumatic encephalopathy) using a TfR-binding peptide oligonucleotide complex described herein. Alzheimer's, Frontotemopral Dementia, and chronic traumatic encephalopathy, exhibit aggregates of Tau protein which generate pro-inflammatory signals and drive neuronal death. The MAPT gene expresses the Tau protein, and reduction of Tau can have utility in CNS disease (e.g., Alzheimer's, Frontotemporal Dementia, and chronic traumatic encephalopathy). The nucleic acid portion of the peptide oligonucleotide complex comprises a gapmer targeting the MAPT gene. Short sequences in the Tau mRNA are identified (e.g., 20 nt sequences in the Tau mRNA), that are greater than 40% G/C in content and complementary sequence to Tau mRNA used in the complex. For example, any 20 mer complementary to the Tau mRNA that has imperfect complementarity (e.g., no more than 85% complementarity, or having at least 3 to 4 mismatches) or no to low complementarity (e.g., no more than 75%, 65%, 50%, or 30% complementarity) relative to other sequences in transcriptome (to reduce off target effects) may be used (e.g., a 20 mer only found in the MAPT gene).

Single stranded structures (e.g., ssRNA or ssDNA) for modulating Tau mRNA can include:

TABLE 15
Examples of Single Stranded Nucleotides
to Modulate Tau mRNA

| SEQ ID NO | Sequence |
|---|---|
| SEQ ID NO: 381 | TGACTTGTCAAGTCATCCTT |
| SEQ ID NO: 382 | GCAGGTTAAGTGATTAACCA |
| SEQ ID NO: 383 | TCCTCTCCACAATTATTGAC |
| SEQ ID NO: 384 | GACGTATTTAGGAGAGGAAG |
| SEQ ID NO: 385 | CTGATACTATGCATGTGGAG |
| SEQ ID NO: 386 | GCCGGGTCATTATTCTTTTT |

Any of SEQ ID NO: 381-SEQ ID NO: 386 may be synthesized as the corresponding RNA sequence, with U substituted for T. The gapmer may bind a target molecule of any one of the MAPT transcript sequences derived from its open reading frame (NCBI Refseq ID NG_011498.1), which could include sequences found in its mature transcripts including NCBI Refseq IDs NM_016835.5 MAPT [organism=Homo sapiens][GeneID=4137][transcript=1], NM_005910.6 MAPT [organism=Homo sapiens][GeneID=4137][transcript=2], NM_016834.5 MAPT [organism=Homo sapiens][GeneID=4137][transcript=3], NM_016841.5 MAPT [organism=Homo sapiens][GeneID=4137][transcript=4], NM 001123067.4 MAPT [organism=Homo sapiens][GeneID=4137][transcript=5], NM_001123066.4 MAPT [organism=Homo sapiens][GeneID=4137][transcript=6], NM 001203251.2 MAPT [organism=Homo sapiens][GeneID=4137][transcript=7], NM 001203252.2 MAPT [organism=Homo sapiens][GeneID=4137][transcript=8], NM 001377265.1 MAPT [organism=Homo sapiens][GeneID=4137][transcript=9], NM 001377266.1 MAPT [organism=Homo sapiens][GeneID=4137][transcript=10], NM_001377267.1 MAPT [organism=Homo sapiens][GeneID=4137][transcript=11], or NM_001377268.1 MAPT [organism=Homo sapiens][GeneID=4137][transcript=12], SEQ ID NO: 397-SEQ ID NO: 408.

For this example, one could construct ASOs with full backbone PS linkages, where all C bases are 5-methyl-C. For this example, the middle 10 nt are DNA sugars and the flanking 5 nt on each side are 2′O-MOE RNA sugars.

The peptide and the oligonucleotide of the TfR-binding peptide oligonucleotide complex of the disclosure are each expressed recombinantly or chemically synthesized and then conjugated together via a linker. Optionally the linker is cleavable. Optionally, the TfR binding peptide has reduced affinity for TfR at pH lower than 7.4. The nucleic acid portion of the peptide oligonucleotide complex is, targeted against any portion of the MAPT pre-mRNA sequence derived from its open reading frame (NCBI Refseq ID NG_011498.1), or a functional fragment thereof including its mature transcripts such as NCBI Refseq IDs NM_016835.5 MAPT [organism=Homo sapiens][GeneID=4137][transcript=1], NM_005910.6 MAPT [organism=Homo sapiens][GeneID=4137][transcript=2], NM_016834.5 MAPT [organism=Homo sapiens][GeneID=4137][transcript=3], NM_016841.5 MAPT [organism=Homo sapiens][GeneID=4137][transcript=4], NM_001123067.4 MAPT [organism=Homo sapiens][GeneID=4137][transcript=5], NM_001123066.4 MAPT [organism=Homo sapiens][GeneID=4137][transcript=6], NM 001203251.2 MAPT [organism=Homo sapiens][GeneID=4137][transcript=7], NM 001203252.2 MAPT [organism=Homo sapiens][GeneID=4137][transcript=8], NM 001377265.1 MAPT [organism=Homo sapiens][GeneID=4137][transcript=9], NM 001377266.1 MAPT [organism=Homo sapiens][GeneID=4137][transcript=10], NM_001377267.1 MAPT [organism=Homo sapiens][GeneID=4137][transcript=11], or NM_001377268.1 MAPT [organism=Homo sapiens][GeneID=4137][transcript=12]. The TfR-binding peptide oligonucleotide complex is administered to a subject in need thereof. The TfR-binding peptide nucleotide complex is administered direct intracranial, intravenously, subcutaneously, intramuscularly, orally, or intrathecally. The subject is a human or an animal. Mouse model experiments may demonstrate pharmacodynamic response upon direct intracranial (control) or IV (experimental) dosage, using any of a number of transgenic MAPT mouse models known to induce CNS disease (e.g., Alzheimer's, Frontotemopral Dementia, and chronic traumatic encephalopathy) in this model. After administration, the TfR-binding peptide nucleotide complex accumulates diseased tissue and the MAPT mRNA is degraded. The TfR-binding peptide nucleotide complex ameliorates the CNS disease (e.g., Alzheimer's, Frontotemporal Dementia, and chronic traumatic encephalopathy), reducing symptoms of CNS disease (e.g., Alzheimer's, Frontotemporal Dementia, and chronic traumatic encephalopathy). Reduced symptoms of tauopathy are exhibited. In patients, symptoms can be cognitive decline, dementia, capabilities at carrying out day to day tasks. In mice, symptoms can be seen in tests for cognitive decline (spatial memory, 8-arm radial maze), motor deficits (walking, rotarod, clasping), hunched posture, Neurofibrillary tangle pathology.

This data demonstrates that the TfR-binding peptide oligonucleotide complexes of the present disclosure effectively treat CNS disease (e.g., Alzheimer's, Frontotemporal Dementia, and chronic traumatic encephalopathy).

### Example 52

### TfR-Binding Peptide Oligonucleotide Complex Using an Anti-miR for Treatment of Solid Tumor

This example describes treatment of Cancers (e.g., glioblastoma multiforme (GBM), pancreatic cancer, breast cancer, colon cancer, lung cancer, head and neck cancer) using a TfR-binding peptide nucleotide complex described herein. Healthy tissues can express tumor suppressor genes such as PDCD4 and PTEN which control cell growth and apoptosis. The miRNA, miR-21 is a repressor of several such tumor suppressor genes, including PDCD4 and PTEN. The reduction of miR-21 hence can have utility in cancers (e.g., GBM, pancreatic cancer, breast cancer, colon cancer, lung cancer, or head and neck cancer) by restoring proper expression of tumor suppressor genes and enabling tumor suppression systems to work. The nucleic acid portion of the peptide nucleotide complex comprises an anti-miR targeting the miR-21 (i.e., anti-miR-21).

Mature miRNA guide strand of miR-21 is as follows: 5′-UAGCUUAUCAGACUGAUGUUGA-3′ (SEQ ID NO: 395). The anti-miR nucleotide may bind a target molecule of SEQ ID NO: 395. Base pairing to an anti-miR sequence would be as follows to generate a complementary anti-MIR-21 nucleic acid:

TABLE 16
Example of MIR-21 miRNA and Anti-miR Base Pairing

| SEQ ID NO: | Sequence | Name |
|---|---|---|
| SEQ ID NO: 395 | 3′-AGUUGUAGUCAGACUAUUCGAU-5′ | miRNA |
| SEQ ID NO: 372 | 5′-UCAACAUCAGUCUGAUAAGCUA-3′ | Full length anti-miR |

The optimal anti-miRNA must match at the seed region, typically sites 2-7 from the miRNA's 5′ end. Hence, truncations to test (to minimize length while maintaining potency) will truncate from the 5′ end of the anti-miR to maintain the 3′ end matching to the miRNA seed sequence:

TABLE 17
Examples of Anti-miR Truncations

| SEQ ID NO: | Sequence | Length |
|---|---|---|
| SEQ ID NO: 372 | UCAACAUCAGUCUGAUAAGCUA | 22 nucleotides |
| SEQ ID NO: 373 | CAACAUCAGUCUGAUAAGCUA | 21 nucleotides |
| SEQ ID NO: 374 | AACAUCAGUCUGAUAAGCUA | 20 nucleotides |
| SEQ ID NO: 375 | ACAUCAGUCUGAUAAGCUA | 19 nucleotides |
| SEQ ID NO: 376 | CAUCAGUCUGAUAAGCUA | 18 nucleotides |
| SEQ ID NO: 377 | AUCAGUCUGAUAAGCUA | 17 nucleotides |
| SEQ ID NO: 378 | UCAGUCUGAUAAGCUA | 16 nucleotides |
| SEQ ID NO: 379 | CAGUCUGAUAAGCUA | 15 nucleotides |

For such an exemplary anti-miR strategy, PO or PS backbone linkages are used; optionally 1-3 terminal linkages are PS. Sugars can be a mixture of DNA, 2′-O-Me, 2′-F, and/or LNA. C bases can be 5-methyl-C.

The peptide and the oligonucleotide of the TfR-binding peptide oligonucleotide complex of the disclosure are expressed recombinantly or chemically synthesized and the conjugated together via a linker. Optionally the linker is cleavable. Optionally the peptide has reduced affinity for TfR at pH less than 7.4. The nucleic acid portion of the peptide oligonucleotide complex is, targeted against any portion of the miR-21 guide strand RNA (SEQ ID NO: 395), or a functional fragment thereof. The TfR-binding peptide oligonucleotide complex is administered to a subject in need thereof. The TfR-binding peptide oligonucleotide complex is administered intravenously, subcutaneously, intramuscularly, orally, intrathecally, intravitreally, or intratumorally. The subject is a human or an animal. Mouse models can include any of a number of xenografts of human tumor lines or primary tumor cells or other relevant cancer models. After administration, the TfR-binding peptide nucleotide complex accumulates diseased tissue and the miR-21 mRNA is degraded. The TfR-binding peptide nucleotide complex causes tumors or cancer cells (e.g., GBM, pancreatic cancer, breast cancer, colon cancer, lung cancer, or head and neck cancer) to grow more slowly, stop growing, or die. Reduced symptoms of cancers (e.g., GBM pancreatic cancer, breast cancer, colon cancer, lung cancer, head and neck cancer) may result. In patients: Reduced symptoms of cancer are exhibited and, reduction of tumor masses and prevention of re-growth (disease control).

This data demonstrates that the TfR-binding peptide oligonucleotide complexes of the present disclosure effectively treat Cancers (e.g., GBM, pancreatic cancer, breast cancer, colon cancer, lung cancer, or head and neck cancer).

### Example 53

### TfR-Binding Peptide Oligonucleotide Complex Using an Aptamer for Treatment of HIV

This example describes treatment of Human Immunodeficiency Virus (HIV) using a TfR-binding peptide oligonucleotide complex described herein. HIV uses CCR5 as a major receptor for infection. With the TfR-binder's bone marrow accumulation ability, bringing a CCR5 inhibiting aptamer to the tissue could reduce HIV proliferation and can therefore have utility in treating HIV. The nucleic acid portion of the peptide oligonucleotide complex comprises an aptamer targeting the CCR5 protein, optionally determined using a SELEX-based screening strategy. CCR5 is membrane-embedded, so soluble CCR5 could be a difficult reagent against which to screen. However, cells or membrane vesicles from cells over-expressing CCR5 could be exposed to a library of 20-40mer sequences of a random nature flanked by a primer-binding site. Cells or vesicles would be rinsed thoroughly and then lysed to release nucleic acids that are bound, which would be amplified by PCR. Negative selection would occur in CCR5-negative material to remove sequences non-specific to CCR5. After several rounds of positive and negative selection and amplification, individual sequences would be synthesized and tested for the ability to bind only to CCR5-expressing cells.

For such a CCR5-targeting aptamer, backbone linkages can be PO or PS; one clinical example of an aptamer, pegaptanib, uses all PO linkages. Sugars could be a mixture of DNA, RNA, 2′-O-Me, 2′-O-MOE, 2′-F, or LNA among others. Optionally, the bases are chemically modified to facilitate tighter binding or even covalent binding.

The peptide and the oligonucleotide of the TfR-binding peptide oligonucleotide complex of the disclosure are expressed recombinantly or chemically synthesized and then conjugated together via a linker. The nucleic acid portion of the peptide oligonucleotide complex is, targeted against any portion of the CCR5 protein, or a functional fragment thereof. The TfR-binding peptide oligonucleotide complex is administered to a subject in need thereof. The TfR-binding peptide oligonucleotide complex is administered intravenously, subcutaneously, intramuscularly, orally, intrathecally, intravitreally, or intratumorally. The subject is a human or an animal. In mouse models, the mice may be immunocompromised and then engrafted with human T cells for HIV infection control testing. After administration, the TfR-binding peptide oligonucleotide complex reduces the ability of HIV to infect or reinfect immune cells. The TfR-binding peptide oligonucleotide complex offers protection from infection and/or reduction of productive infection upon exposure to HIV or HIV-glycoprotein-pseudotyped viral particles. In conjunction with other therapies immune function for HIV patients can be regained and opportunistic infections reduced, enhancing quality of life and potentially increasing life expectancy.

This data demonstrates that the TfR-binding peptide oligonucleotide complexes of the present disclosure effectively treat Human Immunodeficiency Virus (HIV).

### Example 54

### TfR-Binding Peptide Oligonucleotide Complex Using a U1 Adapter for Treatment of Skin Cancer

This example describes treatment of skin cancer (e.g., melanoma) using a TfR-binding peptide oligonucleotide complex described herein. BCL2 is an anti-apoptotic protein implicated in a number of solid tumors. Melanoma, in particular, expresses high levels of BCL2, rendering it resistant to many chemotherapeutics known to induce apoptosis. The BCL2 gene expresses the BCL2 protein, and reduction of BCL2 can have utility in skin cancer (e.g., melanoma). The nucleic acid portion of the peptide oligonucleotide complex which targets the BCL2 gene comprising a complementary nucleotide to BCL2 pre-mRNA linked to a U1 adapter. The 3′ end of the BCL2 pre-mRNA transcript maps to chromosome 18, and polyA mapping software PolyASite identifies the region near Base 63,126,800 (on hg38 genome assembly) as a likely polyA site. Short sequences in the BCL2 gene or pre-mRNA are identified (e.g., overlapping 20 nt sequences in the BCL2 pre-mRNA within 5000 bases on either side of this PolyA region), that are 30%-60% G/C in content and complementary sequence to BCL2 pre-mRNA and placed 5′ or 3′ (this example demonstrates 5′ placement) of a U1-recognition domain used in the complex. For example, any 20 mer complementary to the BCL2 pre-mRNA region that has imperfect complementarity (e.g., no more than 85% complementarity, or having at least 3 to 4 mismatches) or no to low complementarity (e.g., no more than 75%, 65%, 50%, or 30% complementarity) relative to other sequences in transcriptome (to reduce off target effects) may be tested.

An exemplary nucleic acid sequence contains a U1 adapter for modulating BCL2 mRNA that is highly active against BCL2 can include:
5′GCCGUACAGUUCCACAAAGGGCCAGGUAAGUAU-3′ (SEQ ID NO: 380), wherein the underlined portion (GCCGUACAGUUCCACAAAGG; SEQ ID NO: 298) corresponds to the BCL2 recognition sequence and the italicized portion (GCCAGGUAAGUAU; SEQ ID NO: 368) corresponds to the U1 recognition sequence. A U1 adapter may bind a target pre-mRNA molecule derived from the BCL2 open reading frame (NCBI Refseq ID: NG_009361.1). Any of the U1 adapters in TABLE 7 can also be linked to the BCL2 recognition sequence. Sugar modifications may include 2′-O-Me, LNA, or standard RNA or DNA among others. Backbone linkages can include PO or PS linkages.

The peptide and the oligonucleotide of the TfR-binding peptide oligonucleotide complex of the disclosure are expressed recombinantly or chemically synthesized and then conjugated together via a linker. Optionally, the linker is cleavable. Optionally, the peptide has reduced affinity to TfR at pH less than 7.4. The nucleic acid portion of the peptide oligonucleotide complex is, targeted against BCL2 pre-mRNA derived from the BCL2 open reading frame (NCBI Refseq ID: NG_009361.1), or a functional fragment thereof including mRNA NCBI Refseq IDs NM_000633.3 BCL2 [organism=Homo sapiens][GeneID=596][transcript=alpha] or NM_000657.3 BCL2 [organism=Homo sapiens][GeneID=596][transcript=beta], SEQ ID NO: 409, or SEQ ID NO: 410. The TfR-binding peptide oligonucleotide complex is administered to a subject in need thereof. The TfR-binding peptide oligonucleotide complex is administered intravenously, subcutaneously, intramuscularly, orally, intrathecally, intravitreally, or intratumorally. The subject is a human or an animal. In mouse models, one would test in mouse xenograft models with flank tumors of human melanoma cells, or other relevant model. After administration, the TfR-binding peptide nucleotide complex accumulates in diseased tissue and the BCL2 mRNA transcription is reduced and the mRNA degraded, induction of apoptotic markers and reduced tumor growth results in treated animals. The TfR-binding peptide oligonucleotide complex ameliorates the skin cancer (e.g., melanoma). Reduced symptoms of skin cancer (e.g., melanoma) are exhibited.

This data demonstrates that the TfR-binding peptide oligonucleotide complexes of the present disclosure effectively treat skin cancer (e.g., melanoma).

### Example 55

### Design of an Oligonucleotide Sequence for a Peptide Oligonucleotide Complex

This example describes design of an oligonucleotide sequence for a target binding agent capable of binding a target molecule for use in a peptide oligonucleotide complex. A gene is targeted for modulation by a peptide oligonucleotide complex of this disclosure, optionally by a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), complementary oligonucleotide to natural antisense transcripts (NATs) sequences, siRNA, snRNA, gapmer, anti-miR, splice blocker ASO, or U1 Adapter. The gene may be targeted for downregulation to improve a disease condition. Short overlapping sequences (e.g., 12, 15, 20, 21, 25, or 30 nt in length) complementary to the gene, walking along up to the entire length of the gene, are generated and tested to determine which provides the most effective regulation. The sequence may be chosen to contain 3 or more mismatches to other sequences in the transcriptome. The sequence may be chosen to avoid any that have 14 or more matches with a nontarget or undesired complementary sequence. The sequence may be chosen to avoid the most common seed regions of 2-8 nts on the 5′ end of siRNA. Chemical modifications to the oligonucleotide are also tested (concurrently or after sequence testing). Chemical modifications may include modifications on the termini of the oligonucleotides to reduce exonuclease cleavage, such as by placing 1-3 phosphorothioate linkages on all ends. Chemical modifications may include 2′F bases such as 2′F pyrimidine bases for increased stabilization and binding. Chemical modifications may also include 2′-OMe or 2′-Omethoxyethyl bases to decrease immune activation, including to offset that which may be increased by the including of 2′F bases. Chemical modifications may also include using BNA or LNA or any other modification of this disclosure. Optionally, the oligonucleotides are tested in pool, such as 5-10 sequences at once, to narrow down to the best sequences. Optionally, the sequences are also tested for immune activation, such as with an IFIT (Interferon-induced proteins with tetratricopeptide repeats) or T cell activation assay or innate immune activation assay such as qRT-PCR, immune cell activation or proliferation or cytokine secretion, and the sequences with lower immune activation are prioritized. Optionally, nontarget AA/TT sequences are added on the ends of the siRNA. Optionally, sequence overhangs are added on the ends of the siRNA. The oligonucleotide sequences may be selected for homology to both human and other species (such as mouse, rat, and non-human primate). Alternatively, a different oligonucleotide sequence to the same target may be used in other species for preclinical development (e.g., mouse or rat) than the oligonucleotide sequence complementary to the human target which is used for clinical development and to treat human patients. Optionally, an siRNA sequence is designed using the methods of: Fakhr et al. Precise and efficient siRNA design: a key point in competent gene silencing Cancer Gene Therapy. 2016; 23, 73-82.

The oligonucleotide or the peptide oligonucleotide complex is tested for its ability to reduce the level of intact functional RNA or to reduce the level of protein which is encoded by the targeted RNA. The oligonucleotide or the peptide oligonucleotide complex is tested for its ability to generate the desired phenotypic response in the cells, tissue, or animals, such as reduced tumor growth rate, reduced cognitive decline, or reduced inflammation. The oligonucleotide or peptide oligonucleotide complex is also tested for safety or undesirable side effects. The testing is performed in vitro, in vivo, or in humans. The oligonucleotide or the peptide oligonucleotide complex with the most desired attributes is selected.

### Example 56

### Design of an Oligonucleotide Sequence for a Peptide Oligonucleotide Complex

A target gene for making target binding agent capable of binding a target molecule is selected based on the association between its expression and disease; this could be direct (e.g. either the transcript itself or a protein encoded by the transcript is associated with or leads to disease phenotype) or indirect (e.g. either the transcript itself or a protein encoded by that transcript modifies a different gene or transcript or protein whose activity is associated with or leads to disease phenotype). The target sequence is derived from the gene's open reading frame. The target sequence may be found in the coding region or in the non-coding region, and it may be found in the mature mRNA (which has been spliced, polyadenylated, capped, and exported to the cytosol for translation) or in the immature pre-mRNA. The target binding agent will be the complement to such open reading frame. If the target sequence is found in the mature mRNA (for example, when planning to use siRNA), then the search for appropriate sequences will begin with identification of the appropriate transcript isoform, taking into consideration such variables as alternative splicing or alternative transcription start sites. If the target sequence is found in the immature pre-mRNA (for example, when planning to use gapmers, splice-blocking oligonucleotides, or U1 adapters), then the search for appropriate sequences will begin with identification of the full open reading frame of the gene in question, taking into consideration such variables as alternative transcription start sites but with less consideration for alternative splice isoforms. If the target is an antisense sequence (e.g., miRNA to be targeted by an anti-miR), the sequence would be based on the mature guide strand sequence. These reference sequences can be found in public genome databases, including but not limited to the National Center for Biotechnology Information (NCBI) or the University of California Santa Cruz (UCSC) Genome Browser. The pre-mRNA sequence is the same as the genomic sequence. Optionally the reference sequences are as given in TABLE 18.

TABLE 18
Examples of Open Reading Frame Reference Sequences

| | Exemplary | NCBI Refseq | NCBI Refseq |
|---|---|---|---|
| Target | Approach | Gene | RNA(s) |
| IL-23R (gene | siRNA | NG_011498.1 | NM_144701.3 |
| IL23R) | | | |
| Tau (gene | Gapmer | NG_007398.2 | NM_016835.5, |
| MAPT) | | | NM_005910.6, |
| | | | NM_016834.5, |
| | | | NM_001377268.1, |
| | | | NM_016841.5, |
| | | | NM_001123067.4, |
| | | | NM_001123066.4, |
| | | | NM_001203251.2, |
| | | | NM_001203252.2, |
| | | | NM_001377265.1, |
| | | | NM_001377266.1, |
| | | | NM_001377267.1 |
| miR-21 (gene | Anti-miR | | NR_029493.1 (NCBI) |
| MIR21) | | | MIMAT0000076 |
| | | | (miRBase) |
| BCL2 (gene BCL2) | U1 | NG_009361.1 | NM_000633.3, |
| | adapter | | NM_000657.3 |

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-126. (canceled)

127. A peptide oligonucleotide complex comprising a peptide and an oligonucleotide, wherein the peptide comprises a transferrin receptor-binding peptide capable of binding a transferrin receptor, and wherein the oligonucleotide comprises a target-binding agent capable of binding a target molecule.

128. A peptide oligonucleotide complex of claim 127, wherein the oligonucleotide comprises:
a) a U1 adapter;
b) an anti-miR sequence;
c) a small interfering RNA;
d) a gapmer; or
e) an aptamer.

129. The peptide oligonucleotide complex of claim 128, wherein
a) the U1 adaptor comprises a sequence of any one of SEQ ID NO: 364-SEQ ID NO: 371;
b) the anti-miR sequence comprises a sequence of any one of SEQ ID NO: 372-SEQ ID NO: 379;
c) the small interfering RNA comprises a sequence of any one of SEQ ID NO: 387-SEQ ID NO: 394; or
d) the gapmer comprises a sequence of any one of SEQ ID NO: 381-SEQ ID NO: 386.

130. The peptide oligonucleotide complex of claim 127, wherein the target molecule comprises:
a) a molecule listed in TABLE 4, TABLE 5, or TABLE 6;
b) a DNA sequence encoding a molecule listed in TABLE 4, TABLE 5, or TABLE 6; or
c) an RNA sequence encoding a molecule listed in TABLE 4, TABLE 5, or TABLE 6.

131. The peptide oligonucleotide complex of claim 127, wherein the oligonucleotide comprises a nucleotide antisense RNA, a complementary RNA, an inhibitory RNA, an interfering RNA, a nuclear RNA, an antisense oligonucleotide, a microRNA, a sequence complementary to a natural antisense transcript, a small interfering RNA, a small nuclear RNA, an aptamer, a gapmer, an anti-miR sequence, a splice blocker antisense oligonucleotide, or a U1 adapter.

132. The peptide oligonucleotide complex of claim 127, wherein the oligonucleotide comprises at least 90% sequence identity to any one of SEQ ID NO: 364-SEQ ID NO: 394.

133. The peptide oligonucleotide complex of claim 127, wherein the target molecule comprises a sequence of any one of SEQ ID NO: 395-SEQ ID NO: 428 provided in TABLE 3 or an open reading frame listed in TABLE 18, or a fragment thereof.

134. The peptide oligonucleotide complex of claim 127, wherein the target molecule encodes a pro-inflammatory cytokine, an extracellular matrix-modifying protein, TNF-α, ICAM-1, a p65 subunit of NF-κB, Smad7, carbohydrate sulfotransferase 15, IL-23, IL-12, IL-17, poly-Q expanded huntingtin, amyloid precursor protein, microtubule associated protein tau, SMN2, SCN1A, ASO, SCN8A, IGF-1, IGF-1 receptor, EGFR, ERBB3, HER2, GRB2, KRAS, MYC, YAP1, a heat shock protein, a hypoxia-sensing protein, MDM2, BCL2, FOXP3, DNMT1, an HDAC, a parasite surface protein, GPX4, SLC7a11, α-synuclein, a JAK-STAT pathway protein, a viral protein, or LRRK2.

135. The peptide oligonucleotide complex of claim 127, wherein the peptide comprises a sequence having at least 80% sequence identity to any one of SEQ ID NO: 1-SEQ ID NO: 134, a fragment thereof, a variant thereof, a homolog thereof, or an analog thereof.

136. The peptide oligonucleotide complex of claim 127, wherein the peptide comprises a sequence of any one of or SEQ ID NO: 306-SEQ ID NO: 335.

137. The peptide oligonucleotide complex of claim 135, wherein the peptide comprises a sequence of any one of SEQ ID NO: 1-SEQ ID NO: 134.

138. The peptide oligonucleotide complex of claim 127, wherein the peptide comprises a sequence of:
a) SEQ ID NO: 32
b) SEQ ID NO: 2;
c) SEQ ID NO: 64;
d) SEQ ID NO: 34; or
e) any one of SEQ ID NO: 129-SEQ ID NO: 134.

139. The peptide oligonucleotide complex of claim 127, wherein the oligonucleotide is linked to the peptide via a linker.

140. The peptide oligonucleotide complex of claim 139, wherein the linker is selected from TABLE 10, TABLE 11, any one of SEQ ID NO: 234-SEQ ID NO: 297, or combinations thereof.

141. The peptide oligonucleotide complex of claim 127, further comprising an additional cell penetrating moiety, wherein the additional cell penetrating moiety comprises:
a) a polycation, a polyorganic acid, an endosomal releasing polymer, poly(2-propylacrylic acid), poly(2-ethylacrylic acid), a Tat peptide, an Arg patch, a knotted peptide, CysTAT, S19-TAT, R8 (SEQ ID NO: 143), pAntp, Pas-TAT, Pas-R8 (SEQ ID NO: 146), Pas-FHV, Pas-pAntP, F2R4 (SEQ ID NO: 149), B55, aurein, IMT-P8, BR2, OMOTAG1, OMOTAG2, pVEC, SynB3, DPV1047, C105Y, Transportan, MTS, hLF, PFVYLI (SEQ ID NO: 163), maurocalcine, imperatoxin, hadrucalin, hemicalcin, opicalcin-1, opicalcin-2, midkine (62-104), MCoTI-II, chlorotoxin, DRI-TAT, cFΦR4 (SEQ ID NO: 166), R6W3 (SEQ ID NO: 189), myristate, yBBR, or a fragment or variant thereof, or any combination thereof,
b) a sequence of any one of SEQ ID NO: 141-SEQ ID NO: 233; or
c) a combination thereof.

142. The peptide oligonucleotide complex of claim 127 further comprising an active agent, wherein the active agent comprises a radionuclide, a radionuclide chelator, a chelator, an immunotherapeutic agent, a CTLA-4 targeting agent, a PD-1 targeting agent, a PDL-1 targeting agent, an IL15 agent, a fused IL-15/IL-15Ra complex agent, an IFNgamma agent, an anti-CD3 agent, an ion channel modulator, a Kᵥ1.3 inhibitor, an auristatin, MMAE, a maytansinoid, DM1, DM4, doxorubicin, a calicheamicin, a platinum compound, cisplatin, a taxane, paclitaxel, SN-38, a BACE inhibitor, a Bcl-xL inhibitor, WEHI-539, venetoclax, ABT-199, navitoclax, AT-101, obatoclax, a pyrrolobenzodiazepine or pyrrolobenzodiazepine dimer, a dolastatin, or a neurotransmitter.

143. The peptide oligonucleotide complex of claim 127 further comprising a detectable agent, wherein the detectable agent is a fluorophore, a near-infrared dye, a contrast agent, a nanoparticle, a metal-containing nanoparticle, a metal chelate, an X-ray contrast agent, a PET agent, a radionuclide, or a radionuclide chelator.

144. A method of modulating an activity of a target molecule, the method comprising:
contacting a cell with a peptide oligonucleotide complex of claim 127,
binding the peptide to a transferrin receptor;
transporting the peptide oligonucleotide complex across a cellular layer of the cell; and
binding the oligonucleotide to a target molecule, thereby modulating the activity of the target molecule.

145. A method of treating a condition in a subject in need thereof, the method comprising:
administering to the subject a composition comprising a peptide oligonucleotide complex of claim 127;
binding the peptide to a transferrin receptor;
delivering the peptide oligonucleotide complex across a cellular layer of the subject;
binding the nucleotide to a target molecule; and
modulating an activity of the target molecule associated with the condition, thereby treating the condition in the subject.

146. The method of claim 145, wherein the condition is a neuronal condition, a gastrointestinal condition, an inflammatory condition, an immune condition, a neurological condition, a muscular condition, an infectious condition, or a cancer.


SEQUENCE LISTING (432 sequences)

SEQ ID NO: 1 - protein, 49 aa, synthetic construct
REGCASRCTKYNAELEKCEARVSSMSNTEETCVQELFDLLHCVDHCVSQ

SEQ ID NO: 2 - protein, 49 aa, synthetic construct
REGCASRCTKYNAELEKCEARVMSMSNTEEDCEQELEDLLHCLDHCHSQ

SEQ ID NO: 3 - protein, 49 aa, synthetic construct
REGCASRCTKYNAELEKCEARVVSMSNTEEDCEQELEDLLHCLDHCHSQ

SEQ ID NO: 4 - protein, 49 aa, synthetic construct
REGCASRCTKYNAELEKCEARVMSMSNTEETCEQELEDLLHCLDHCHSQ

SEQ ID NO: 5 - protein, 49 aa, synthetic construct
REGCASRCTKYNAELEKCEARVMSMSNTEEDCVQELEDLLHCLDHCHSQ

SEQ ID NO: 6 - protein, 49 aa, synthetic construct
REGCASRCTKYNAELEKCEARVMSMSNTEEDCEQELFDLLHCLDHCHSQ

SEQ ID NO: 7 - protein, 49 aa, synthetic construct
REGCASRCTKYNAELEKCEARVMSMSNTEEDCEQELEDLLHCVDHCHSQ

SEQ ID NO: 8 - protein, 49 aa, synthetic construct
REGCASRCTKYNAELEKCEARVMSMSNTEEDCEQELEDLLHCLDHCVSQ

SEQ ID NO: 9 - protein, 49 aa, synthetic construct
REGCASRCTKYNAELEKCEARVVSMSNTEETCEQELEDLLHCLDHCHSQ

SEQ ID NO: 10 - protein, 49 aa, synthetic construct
REGCASRCTKYNAELEKCEARVVSMSNTEEDCVQELEDLLHCLDHCHSQ

SEQ ID NO: 11 - protein, 49 aa, synthetic construct
REGCASRCTKYNAELEKCEARVVSMSNTEEDCEQELFDLLHCLDHCHSQ

SEQ ID NO: 12 - protein, 49 aa, synthetic construct
REGCASRCTKYNAELEKCEARVVSMSNTEEDCEQELEDLLHCVDHCHSQ

SEQ ID NO: 13 - protein, 49 aa, synthetic construct
REGCASRCTKYNAELEKCEARVVSMSNTEEDCEQELEDLLHCLDHCVSQ

SEQ ID NO: 14 - protein, 49 aa, synthetic construct
REGCASRCTKYNAELEKCEARVMSMSNTEETCVQELEDLLHCLDHCHSQ

SEQ ID NO: 15 - protein, 49 aa, synthetic construct
REGCASRCTKYNAELEKCEARVMSMSNTEETCEQELFDLLHCLDHCHSQ

SEQ ID NO: 16 - protein, 49 aa, synthetic construct
REGCASRCTKYNAELEKCEARVMSMSNTEETCEQELEDLLHCVDHCHSQ

SEQ ID NO: 17 - protein, 49 aa, synthetic construct
REGCASRCTKYNAELEKCEARVMSMSNTEETCEQELEDLLHCLDHCVSQ

SEQ ID NO: 18 - protein, 49 aa, synthetic construct
REGCASRCTKYNAELEKCEARVMSMSNTEEDCVQELFDLLHCLDHCHSQ

SEQ ID NO: 19 - protein, 49 aa, synthetic construct
REGCASRCTKYNAELEKCEARVMSMSNTEEDCVQELEDLLHCVDHCHSQ

SEQ ID NO: 20 - protein, 49 aa, synthetic construct
REGCASRCTKYNAELEKCEARVMSMSNTEEDCVQELEDLLHCLDHCVSQ

SEQ ID NO: 21 - protein, 49 aa, synthetic construct
REGCASRCTKYNAELEKCEARVMSMSNTEEDCEQELFDLLHCVDHCHSQ

SEQ ID NO: 22 - protein, 49 aa, synthetic construct
REGCASRCTKYNAELEKCEARVMSMSNTEEDCEQELFDLLHCLDHCVSQ

SEQ ID NO: 23 - protein, 49 aa, synthetic construct
REGCASRCTKYNAELEKCEARVMSMSNTEEDCEQELEDLLHCVDHCVSQ

SEQ ID NO: 24 - protein, 49 aa, synthetic construct
REGCASRCMKYNAELEKCEARVMSMSNTEEDCEQELEDLLHCLDHCHSQ

SEQ ID NO: 25 - protein, 49 aa, synthetic construct
REGCASRCTKYNDELEKCEARVMSMSNTEEDCEQELEDLLHCLDHCHSQ

SEQ ID NO: 26 - protein, 49 aa, synthetic construct
REGCASRCTKYNAELEKCEARMMSMSNTEEDCEQELEDLLHCLDHCHSQ

SEQ ID NO: 27 - protein, 49 aa, synthetic construct
REGCASRCTKYNAELEKCEARVMSMSNTEEDCEQELEDLLYCLDHCHSQ

SEQ ID NO: 28 - protein, 49 aa, synthetic construct
REGCASRCMKYNDELEKCEARMMSMSNTEEDCEQELEDLLHCLDHCHSQ

SEQ ID NO: 29 - protein, 49 aa, synthetic construct
REGCASRCMKYNDELEKCEARVMSMSNTEEDCEQELEDLLYCLDHCHSQ

SEQ ID NO: 30 - protein, 49 aa, synthetic construct
REGCASRCMKYNAELEKCEARMMSMSNTEEDCEQELEDLLYCLDHCHSQ

SEQ ID NO: 31 - protein, 49 aa, synthetic construct
REGCASRCTKYNDELEKCEARMMSMSNTEEDCEQELEDLLYCLDHCHSQ

SEQ ID NO: 32 - protein, 49 aa, synthetic construct
REGCASRCMKYNDELEKCEARMMSMSNTEEDCEQELEDLLYCLDHCHSQ

SEQ ID NO: 33 - protein, 49 aa, synthetic construct
REGCASRCTRYNAELERCEARVSSMSNTEETCVQELFDLLHCVDHCVSQ

SEQ ID NO: 34 - protein, 49 aa, synthetic construct
REGCASRCTRYNAELERCEARVMSMSNTEEDCEQELEDLLHCLDHCHSQ

SEQ ID NO: 35 - protein, 49 aa, synthetic construct
REGCASRCTRYNAELERCEARVVSMSNTEEDCEQELEDLLHCLDHCHSQ

SEQ ID NO: 36 - protein, 49 aa, synthetic construct
REGCASRCTRYNAELERCEARVMSMSNTEETCEQELEDLLHCLDHCHSQ

SEQ ID NO: 37 - protein, 49 aa, synthetic construct
REGCASRCTRYNAELERCEARVMSMSNTEEDCVQELEDLLHCLDHCHSQ

SEQ ID NO: 38 - protein, 49 aa, synthetic construct
REGCASRCTRYNAELERCEARVMSMSNTEEDCEQELFDLLHCLDHCHSQ

SEQ ID NO: 39 - protein, 49 aa, synthetic construct
REGCASRCTRYNAELERCEARVMSMSNTEEDCEQELEDLLHCVDHCHSQ

SEQ ID NO: 40 - protein, 49 aa, synthetic construct
REGCASRCTRYNAELERCEARVMSMSNTEEDCEQELEDLLHCLDHCVSQ

SEQ ID NO: 41 - protein, 49 aa, synthetic construct
REGCASRCTRYNAELERCEARVVSMSNTEETCEQELEDLLHCLDHCHSQ

SEQ ID NO: 42 - protein, 49 aa, synthetic construct
REGCASRCTRYNAELERCEARVVSMSNTEEDCVQELEDLLHCLDHCHSQ

SEQ ID NO: 43 - protein, 49 aa, synthetic construct
REGCASRCTRYNAELERCEARVVSMSNTEEDCEQELFDLLHCLDHCHSQ

SEQ ID NO: 44 - protein, 49 aa, synthetic construct
REGCASRCTRYNAELERCEARVVSMSNTEEDCEQELEDLLHCVDHCHSQ

SEQ ID NO: 45 - protein, 49 aa, synthetic construct
REGCASRCTRYNAELERCEARVVSMSNTEEDCEQELEDLLHCLDHCVSQ

SEQ ID NO: 46 - protein, 49 aa, synthetic construct
REGCASRCTRYNAELERCEARVMSMSNTEETCVQELEDLLHCLDHCHSQ

SEQ ID NO: 47 - protein, 49 aa, synthetic construct
REGCASRCTRYNAELERCEARVMSMSNTEETCEQELFDLLHCLDHCHSQ

SEQ ID NO: 48 - protein, 49 aa, synthetic construct
REGCASRCTRYNAELERCEARVMSMSNTEETCEQELEDLLHCVDHCHSQ

SEQ ID NO: 49 - protein, 49 aa, synthetic construct
REGCASRCTRYNAELERCEARVMSMSNTEETCEQELEDLLHCLDHCVSQ

SEQ ID NO: 50 - protein, 49 aa, synthetic construct
REGCASRCTRYNAELERCEARVMSMSNTEEDCVQELFDLLHCLDHCHSQ

SEQ ID NO: 51 - protein, 49 aa, synthetic construct
REGCASRCTRYNAELERCEARVMSMSNTEEDCVQELEDLLHCVDHCHSQ

SEQ ID NO: 52 - protein, 49 aa, synthetic construct
REGCASRCTRYNAELERCEARVMSMSNTEEDCVQELEDLLHCLDHCVSQ

SEQ ID NO: 53 - protein, 49 aa, synthetic construct
REGCASRCTRYNAELERCEARVMSMSNTEEDCEQELFDLLHCVDHCHSQ

SEQ ID NO: 54 - protein, 49 aa, synthetic construct
REGCASRCTRYNAELERCEARVMSMSNTEEDCEQELFDLLHCLDHCVSQ

SEQ ID NO: 55 - protein, 49 aa, synthetic construct
REGCASRCTRYNAELERCEARVMSMSNTEEDCEQELEDLLHCVDHCVSQ

SEQ ID NO: 56 - protein, 49 aa, synthetic construct
REGCASRCMRYNAELERCEARVMSMSNTEEDCEQELEDLLHCLDHCHSQ

SEQ ID NO: 57 - protein, 49 aa, synthetic construct
REGCASRCTRYNDELERCEARVMSMSNTEEDCEQELEDLLHCLDHCHSQ

SEQ ID NO: 58 - protein, 49 aa, synthetic construct
REGCASRCTRYNAELERCEARMMSMSNTEEDCEQELEDLLHCLDHCHSQ

SEQ ID NO: 59 - protein, 49 aa, synthetic construct
REGCASRCTRYNAELERCEARVMSMSNTEEDCEQELEDLLYCLDHCHSQ

SEQ ID NO: 60 - protein, 49 aa, synthetic construct
REGCASRCMRYNDELERCEARMMSMSNTEEDCEQELEDLLHCLDHCHSQ

SEQ ID NO: 61 - protein, 49 aa, synthetic construct
REGCASRCMRYNDELERCEARVMSMSNTEEDCEQELEDLLYCLDHCHSQ

SEQ ID NO: 62 - protein, 49 aa, synthetic construct
REGCASRCMRYNAELERCEARMMSMSNTEEDCEQELEDLLYCLDHCHSQ

SEQ ID NO: 63 - protein, 49 aa, synthetic construct
REGCASRCTRYNDELERCEARMMSMSNTEEDCEQELEDLLYCLDHCHSQ

SEQ ID NO: 64 - protein, 49 aa, synthetic construct
REGCASRCMRYNDELERCEARMMSMSNTEEDCEQELEDLLYCLDHCHSQ

SEQ ID NO: 65 - protein, 51 aa, synthetic construct
GSREGCASRCTKYNAELEKCEARVSSMSNTEETCVQELFDLLHCVDHCVSQ

SEQ ID NO: 66 - protein, 51 aa, synthetic construct
GSREGCASRCTKYNAELEKCEARVMSMSNTEEDCEQELEDLLHCLDHCHSQ

SEQ ID NO: 67 - protein, 51 aa, synthetic construct
GSREGCASRCTKYNAELEKCEARVVSMSNTEEDCEQELEDLLHCLDHCHSQ

SEQ ID NO: 68 - protein, 51 aa, synthetic construct
GSREGCASRCTKYNAELEKCEARVMSMSNTEETCEQELEDLLHCLDHCHSQ

SEQ ID NO: 69 - protein, 51 aa, synthetic construct
GSREGCASRCTKYNAELEKCEARVMSMSNTEEDCVQELEDLLHCLDHCHSQ

SEQ ID NO: 70 - protein, 51 aa, synthetic construct
GSREGCASRCTKYNAELEKCEARVMSMSNTEEDCEQELFDLLHCLDHCHSQ

SEQ ID NO: 71 - protein, 51 aa, synthetic construct
GSREGCASRCTKYNAELEKCEARVMSMSNTEEDCEQELEDLLHCVDHCHSQ

SEQ ID NO: 72 - protein, 51 aa, synthetic construct
GSREGCASRCTKYNAELEKCEARVMSMSNTEEDCEQELEDLLHCLDHCVSQ

SEQ ID NO: 73 - protein, 51 aa, synthetic construct
GSREGCASRCTKYNAELEKCEARVVSMSNTEETCEQELEDLLHCLDHCHSQ

SEQ ID NO: 74 - protein, 51 aa, synthetic construct
GSREGCASRCTKYNAELEKCEARVVSMSNTEEDCVQELEDLLHCLDHCHSQ

SEQ ID NO: 75 - protein, 51 aa, synthetic construct
GSREGCASRCTKYNAELEKCEARVVSMSNTEEDCEQELFDLLHCLDHCHSQ

SEQ ID NO: 76 - protein, 51 aa, synthetic construct
GSREGCASRCTKYNAELEKCEARVVSMSNTEEDCEQELEDLLHCVDHCHSQ

SEQ ID NO: 77 - protein, 51 aa, synthetic construct
GSREGCASRCTKYNAELEKCEARVVSMSNTEEDCEQELEDLLHCLDHCVSQ

SEQ ID NO: 78 - protein, 51 aa, synthetic construct
GSREGCASRCTKYNAELEKCEARVMSMSNTEETCVQELEDLLHCLDHCHSQ

SEQ ID NO: 79 - protein, 51 aa, synthetic construct
GSREGCASRCTKYNAELEKCEARVMSMSNTEETCEQELFDLLHCLDHCHSQ

SEQ ID NO: 80 - protein, 51 aa, synthetic construct
GSREGCASRCTKYNAELEKCEARVMSMSNTEETCEQELEDLLHCVDHCHSQ

SEQ ID NO: 81 - protein, 51 aa, synthetic construct
GSREGCASRCTKYNAELEKCEARVMSMSNTEETCEQELEDLLHCLDHCVSQ

SEQ ID NO: 82 - protein, 51 aa, synthetic construct
GSREGCASRCTKYNAELEKCEARVMSMSNTEEDCVQELFDLLHCLDHCHSQ

SEQ ID NO: 83 - protein, 51 aa, synthetic construct
GSREGCASRCTKYNAELEKCEARVMSMSNTEEDCVQELEDLLHCVDHCHSQ

SEQ ID NO: 84 - protein, 51 aa, synthetic construct
GSREGCASRCTKYNAELEKCEARVMSMSNTEEDCVQELEDLLHCLDHCVSQ

SEQ ID NO: 85 - protein, 51 aa, synthetic construct
GSREGCASRCTKYNAELEKCEARVMSMSNTEEDCEQELFDLLHCVDHCHSQ

SEQ ID NO: 86 - protein, 51 aa, synthetic construct
GSREGCASRCTKYNAELEKCEARVMSMSNTEEDCEQELFDLLHCLDHCVSQ

SEQ ID NO: 87 - protein, 51 aa, synthetic construct
GSREGCASRCTKYNAELEKCEARVMSMSNTEEDCEQELEDLLHCVDHCVSQ

SEQ ID NO: 88 - protein, 51 aa, synthetic construct
GSREGCASRCMKYNAELEKCEARVMSMSNTEEDCEQELEDLLHCLDHCHSQ

SEQ ID NO: 89 - protein, 51 aa, synthetic construct
GSREGCASRCTKYNDELEKCEARVMSMSNTEEDCEQELEDLLHCLDHCHSQ

SEQ ID NO: 90 - protein, 51 aa, synthetic construct
GSREGCASRCTKYNAELEKCEARMMSMSNTEEDCEQELEDLLHCLDHCHSQ

SEQ ID NO: 91 - protein, 51 aa, synthetic construct
GSREGCASRCTKYNAELEKCEARVMSMSNTEEDCEQELEDLLYCLDHCHSQ

SEQ ID NO: 92 - protein, 51 aa, synthetic construct
GSREGCASRCMKYNDELEKCEARMMSMSNTEEDCEQELEDLLHCLDHCHSQ

SEQ ID NO: 93 - protein, 51 aa, synthetic construct
GSREGCASRCMKYNDELEKCEARVMSMSNTEEDCEQELEDLLYCLDHCHSQ

SEQ ID NO: 94 - protein, 51 aa, synthetic construct
GSREGCASRCMKYNAELEKCEARMMSMSNTEEDCEQELEDLLYCLDHCHSQ

SEQ ID NO: 95 - protein, 51 aa, synthetic construct
GSREGCASRCTKYNDELEKCEARMMSMSNTEEDCEQELEDLLYCLDHCHSQ

SEQ ID NO: 96 - protein, 51 aa, synthetic construct
GSREGCASRCMKYNDELEKCEARMMSMSNTEEDCEQELEDLLYCLDHCHSQ

SEQ ID NO: 97 - protein, 51 aa, synthetic construct
GSREGCASRCTRYNAELERCEARVSSMSNTEETCVQELFDLLHCVDHCVSQ

SEQ ID NO: 98 - protein, 51 aa, synthetic construct
GSREGCASRCTRYNAELERCEARVMSMSNTEEDCEQELEDLLHCLDHCHSQ

SEQ ID NO: 99 - protein, 51 aa, synthetic construct
GSREGCASRCTRYNAELERCEARVVSMSNTEEDCEQELEDLLHCLDHCHSQ

SEQ ID NO: 100 - protein, 51 aa, synthetic construct
GSREGCASRCTRYNAELERCEARVMSMSNTEETCEQELEDLLHCLDHCHSQ

SEQ ID NO: 101 - protein, 51 aa, synthetic construct
GSREGCASRCTRYNAELERCEARVMSMSNTEEDCVQELEDLLHCLDHCHSQ

SEQ ID NO: 102 - protein, 51 aa, synthetic construct
GSREGCASRCTRYNAELERCEARVMSMSNTEEDCEQELFDLLHCLDHCHSQ

SEQ ID NO: 103 - protein, 51 aa, synthetic construct
GSREGCASRCTRYNAELERCEARVMSMSNTEEDCEQELEDLLHCVDHCHSQ

SEQ ID NO: 104 - protein, 51 aa, synthetic construct
GSREGCASRCTRYNAELERCEARVMSMSNTEEDCEQELEDLLHCLDHCVSQ

SEQ ID NO: 105 - protein, 51 aa, synthetic construct
GSREGCASRCTRYNAELERCEARVVSMSNTEETCEQELEDLLHCLDHCHSQ

SEQ ID NO: 106 - protein, 51 aa, synthetic construct
GSREGCASRCTRYNAELERCEARVVSMSNTEEDCVQELEDLLHCLDHCHSQ

SEQ ID NO: 107 - protein, 51 aa, synthetic construct
GSREGCASRCTRYNAELERCEARVVSMSNTEEDCEQELFDLLHCLDHCHSQ

SEQ ID NO: 108 - protein, 51 aa, synthetic construct
GSREGCASRCTRYNAELERCEARVVSMSNTEEDCEQELEDLLHCVDHCHSQ

SEQ ID NO: 109 - protein, 51 aa, synthetic construct
GSREGCASRCTRYNAELERCEARVVSMSNTEEDCEQELEDLLHCLDHCVSQ

SEQ ID NO: 110 - protein, 51 aa, synthetic construct
GSREGCASRCTRYNAELERCEARVMSMSNTEETCVQELEDLLHCLDHCHSQ

SEQ ID NO: 111 - protein, 51 aa, synthetic construct
GSREGCASRCTRYNAELERCEARVMSMSNTEETCEQELFDLLHCLDHCHSQ

SEQ ID NO: 112 - protein, 51 aa, synthetic construct
GSREGCASRCTRYNAELERCEARVMSMSNTEETCEQELEDLLHCVDHCHSQ

SEQ ID NO: 113 - protein, 51 aa, synthetic construct
GSREGCASRCTRYNAELERCEARVMSMSNTEETCEQELEDLLHCLDHCVSQ

SEQ ID NO: 114 - protein, 51 aa, synthetic construct
GSREGCASRCTRYNAELERCEARVMSMSNTEEDCVQELFDLLHCLDHCHSQ

SEQ ID NO: 115 - protein, 51 aa, synthetic construct
GSREGCASRCTRYNAELERCEARVMSMSNTEEDCVQELEDLLHCVDHCHSQ

SEQ ID NO: 116 - protein, 51 aa, synthetic construct
GSREGCASRCTRYNAELERCEARVMSMSNTEEDCVQELEDLLHCLDHCVSQ

SEQ ID NO: 117 - protein, 51 aa, synthetic construct
GSREGCASRCTRYNAELERCEARVMSMSNTEEDCEQELFDLLHCVDHCHSQ

SEQ ID NO: 118 - protein, 51 aa, synthetic construct
GSREGCASRCTRYNAELERCEARVMSMSNTEEDCEQELFDLLHCLDHCVSQ

SEQ ID NO: 119 - protein, 51 aa, synthetic construct
GSREGCASRCTRYNAELERCEARVMSMSNTEEDCEQELEDLLHCVDHCVSQ

SEQ ID NO: 120 - protein, 51 aa, synthetic construct
GSREGCASRCMRYNAELERCEARVMSMSNTEEDCEQELEDLLHCLDHCHSQ

SEQ ID NO: 121 - protein, 51 aa, synthetic construct
GSREGCASRCTRYNDELERCEARVMSMSNTEEDCEQELEDLLHCLDHCHSQ

SEQ ID NO: 122 - protein, 51 aa, synthetic construct
GSREGCASRCTRYNAELERCEARMMSMSNTEEDCEQELEDLLHCLDHCHSQ

SEQ ID NO: 123 - protein, 51 aa, synthetic construct
GSREGCASRCTRYNAELERCEARVMSMSNTEEDCEQELEDLLYCLDHCHSQ

SEQ ID NO: 124 - protein, 51 aa, synthetic construct
GSREGCASRCMRYNDELERCEARMMSMSNTEEDCEQELEDLLHCLDHCHSQ

SEQ ID NO: 125 - protein, 51 aa, synthetic construct
GSREGCASRCMRYNDELERCEARVMSMSNTEEDCEQELEDLLYCLDHCHSQ

SEQ ID NO: 126 - protein, 51 aa, synthetic construct
GSREGCASRCMRYNAELERCEARMMSMSNTEEDCEQELEDLLYCLDHCHSQ

SEQ ID NO: 127 - protein, 51 aa, synthetic construct
GSREGCASRCTRYNDELERCEARMMSMSNTEEDCEQELEDLLYCLDHCHSQ

SEQ ID NO: 128 - protein, 51 aa, synthetic construct
GSREGCASRCMRYNDELERCEARMMSMSNTEEDCEQELEDLLYCLDHCHSQ

SEQ ID NO: 129 - protein, 50 aa, synthetic construct
  SITE 1: note = 6-azidohexanoyl
XREGCASRCMKYNDELEKCEARMMSMSNTEEDCEQELEDLLYCLDHCHSQ

SEQ ID NO: 130 - protein, 50 aa, synthetic construct
  SITE 1: note = 6-azidohexanoyl
XREGCASRCTKYNAELEKCEARVMSMSNTEEDCEQELEDLLHCLDHCHSQ

SEQ ID NO: 131 - protein, 54 aa, synthetic construct
  SITE 1: note = 6-azidohexanoyl
  SITE 4: note = citrulline
XGVXGREGCASRCMKYNDELEKCEARMMSMSNTEEDCEQELEDLLYCLDHCHSQ

SEQ ID NO: 132 - protein, 51 aa, synthetic construct
  SITE 1: note = 6-azidohexanoyl
XGREGCASRCMKYNDELEKCEARMMSMSNTEEDCEQELEDLLYCLDHCHSQ

SEQ ID NO: 133 - protein, 51 aa, synthetic construct
  SITE 1: note = 6-azidohexanoyl
XGREGCASRCTKYNAELEKCEARVMSMSNTEEDCEQELEDLLHCLDHCHSQ

SEQ ID NO: 134 - protein, 55 aa, synthetic construct
  SITE 1: note = 6-azidohexanoyl
  SITE 4: note = citrulline
XGVXGGREGCASRCMKYNDELEKCEARMMSMSNTEEDCEQELEDLLYCLDHCHSQ

SEQ ID NO: 135 - protein, 65 aa, synthetic construct
REGCASRCTKYNAELEKCEARVSSMSNTEETCVQELFDLLHCVDHCVSQGSSELYENKPRRPYIL

SEQ ID NO: 136 - protein, 65 aa, synthetic construct
REGCASRCTKYNAELEKCEARVMSMSNTEEDCEQELEDLLHCLDHCHSQGSSELYENKPRRPYIL

SEQ ID NO: 137 - protein, 65 aa, synthetic construct
REGCASRCMKYNDELEKCEARMMSMSNTEEDCEQELEDLLYCLDHCHSQGSSELYENKPRRPYIL

SEQ ID NO: 138 - protein, 67 aa, synthetic construct
GSREGCASRCTKYNAELEKCEARVSSMSNTEETCVQELFDLLHCVDHCVSQGSSELYENKPRRPYIL

SEQ ID NO: 139 - protein, 67 aa, synthetic construct
GSREGCASRCTKYNAELEKCEARVMSMSNTEEDCEQELEDLLHCLDHCHSQGSSELYENKPRRPYIL

SEQ ID NO: 140 - protein, 67 aa, synthetic construct
GSREGCASRCMKYNDELEKCEARMMSMSNTEEDCEQELEDLLYCLDHCHSQGSSELYENKPRRPYIL

SEQ ID NO: 141 - protein, 11 aa, synthetic construct
CYRKKRRQRRR

SEQ ID NO: 142 - protein, 29 aa, synthetic construct
PFVIGAGVLGALGTGIGGIGRKKRRQRRR

SEQ ID NO: 143 - protein, 8 aa, synthetic construct
RRRRRRRR

SEQ ID NO: 144 - protein, 16 aa, synthetic construct
RQIKIWFQNRRMKWKK

SEQ ID NO: 145 - protein, 17 aa, synthetic construct
FFLIPKGGRKKRRQRRR

SEQ ID NO: 146 - protein, 15 aa, synthetic construct
FFLIPKGRRRRRRRR

SEQ ID NO: 147 - protein, 22 aa, synthetic construct
FFLIPKGRRRRNRTRRNRRRVR

SEQ ID NO: 148 - protein, 23 aa, synthetic construct
FFLIPKGRQIKIWFQNRRMKWKK

SEQ ID NO: 149 - protein, 6 aa, synthetic construct
FFRRRR

SEQ ID NO: 150 - protein, 55 aa, synthetic construct
KAVLGATKIDLPVDINDPYDLGLLLRHLRHHSNLLANIGDPAVREQVLSAMQEEE

SEQ ID NO: 151 - protein, 28 aa, synthetic construct
LSTAADMQGVVTDGMASGLDKDYLKPDD

SEQ ID NO: 152 - protein, 15 aa, synthetic construct
RRWRRWNRFNRRRCR

SEQ ID NO: 153 - protein, 17 aa, synthetic construct
RAGLQFPVGRLLRRLLR

SEQ ID NO: 154 - protein, 13 aa, synthetic construct
KRAHHNALERKRR

SEQ ID NO: 155 - protein, 13 aa, synthetic construct
RRMKANARERNRM

SEQ ID NO: 156 - protein, 18 aa, synthetic construct
LLIILRRRIRKQAHAHSK

SEQ ID NO: 157 - protein, 10 aa, synthetic construct
RRLSYSRRRF

SEQ ID NO: 158 - protein, 19 aa, synthetic construct
VKRGLKLRHVRPRVTRMDV

SEQ ID NO: 159 - protein, 17 aa, synthetic construct
CSIPPEVKFNKPFVYLI

SEQ ID NO: 160 - protein, 27 aa, synthetic construct
GWTLNSAGYLLGKINLKALAALAKKIL

SEQ ID NO: 161 - protein, 17 aa, synthetic construct
KGEGAAVLLPVLLAAPG

SEQ ID NO: 162 - protein, 22 aa, synthetic construct
KCFQWQRNMRKVRGPPVSCIKR

SEQ ID NO: 163 - protein, 6 aa, synthetic construct
PFVYLI

SEQ ID NO: 164 - protein, 12 aa, synthetic construct
VLDSLEFIASKL

SEQ ID NO: 165 - protein, 11 aa, synthetic construct
  SITE 1: note = D-arginine
  SITE 2: note = D-arginine
  SITE 3: note = D-arginine
  SITE 4: note = D-glutamine
  SITE 5: note = D-arginine
  SITE 6: note = D-arginine
  SITE 7: note = D-lysine
  SITE 8: note = D-lysine
  SITE 9: note = D-arginine
  SITE 10: note = D-glycine
  SITE 11: note = D-tyrosine
RRRQRRKKRGY

SEQ ID NO: 166 - protein, 7 aa, synthetic construct
  SITE 2: note = l-2-naphthylalanine
FARRRRQ

SEQ ID NO: 167 - protein, 10 aa, synthetic construct
  VARIANT 1..10: note = This sequence may encompass 2-10 residues
RRRRRRRRRR

SEQ ID NO: 168 - protein, 11 aa, synthetic construct
YGRKKRRQRRR

SEQ ID NO: 169 - protein, 10 aa, synthetic construct
GRKKRRQRRR

SEQ ID NO: 170 - protein, 12 aa, synthetic construct
GRKKRRQRRRPQ

SEQ ID NO: 171 - protein, 16 aa, synthetic construct
RKKRRRESRKKRRRES

SEQ ID NO: 172 - protein, 18 aa, synthetic construct
RKGFYKRKQCKPSRGRKR

SEQ ID NO: 173 - protein, 34 aa, synthetic construct
NAATATRGRSAASRPTQRPRAPARSASRPRRPVQ

SEQ ID NO: 174 - protein, 21 aa, synthetic construct
AGYLLGKINLKALAALAKKIL

SEQ ID NO: 175 - protein, 18 aa, synthetic construct
KLALKLALKALKAALKLA

SEQ ID NO: 176 - protein, 28 aa, synthetic construct
MVKSKIGSWILVLFVAMWSDVGLCKKRP

SEQ ID NO: 177 - protein, 22 aa, synthetic construct
MVRRFLVTLRIRRACGPPRVRV

SEQ ID NO: 178 - protein, 30 aa, synthetic construct
WEAALAEALAEALAEHLAEALAEALEALAA

SEQ ID NO: 179 - protein, 18 aa, synthetic construct
VRLPPPVRLPPPVRLPPP

SEQ ID NO: 180 - protein, 27 aa, synthetic construct
GLAFLGFLGAAGSTMGAWSQPKKKRKV

SEQ ID NO: 181 - protein, 21 aa, synthetic construct
KETWWETWWTEWSQPKKKRKV

SEQ ID NO: 182 - protein, 8 aa, synthetic construct
WRWRWRWR

SEQ ID NO: 183 - protein, 23 aa, synthetic construct
MGLGLHLLVLAAALQGAKKKRKV

SEQ ID NO: 184 - protein, 17 aa, synthetic construct
AAVALLPAVLLAHLLAP

SEQ ID NO: 185 - protein, 26 aa, synthetic construct
GIGAVLKVLTTGLPALISWIKRKRQQ

SEQ ID NO: 186 - protein, 20 aa, synthetic construct
HGLASTLTRWAHYNALIRAF

SEQ ID NO: 187 - protein, 13 aa, synthetic construct
GRKKRRQRRRPPQ

SEQ ID NO: 188 - protein, 27 aa, synthetic construct
GALFLGFLGAAGSTMGAWSQPKKKRKV

SEQ ID NO: 189 - protein, 9 aa, synthetic construct
RRWWRRWRR

SEQ ID NO: 190 - protein, 13 aa, synthetic construct
CGYGPKKKRKVGG

SEQ ID NO: 191 - protein, 8 aa, synthetic construct
KKKKKKKK

SEQ ID NO: 192 - protein, 10 aa, synthetic construct
RRIPNRRPRR

SEQ ID NO: 193 - protein, 5 aa, synthetic construct
RLRWR

SEQ ID NO: 194 - protein, 20 aa, synthetic construct
KETWWETWWTEWSQPKKRKV

SEQ ID NO: 195 - protein, 18 aa, synthetic construct
QLALQLALQALQAALQLA

SEQ ID NO: 196 - protein, 26 aa, synthetic construct
DPKGDPKGVTVTVTVTVTGKGDPKPD

SEQ ID NO: 197 - protein, 24 aa, synthetic construct
RRIRPRPPRLPRPRPRPLPFPRPG

SEQ ID NO: 198 - protein, 9 aa, synthetic construct
PPRPPRPPR

SEQ ID NO: 199 - protein, 12 aa, synthetic construct
PPRPPRPPRPPR

SEQ ID NO: 200 - protein, 15 aa, synthetic construct
PPRPPRPPRPPRPPR

SEQ ID NO: 201 - protein, 18 aa, synthetic construct
PPRPPRPPRPPRPPRPPR

SEQ ID NO: 202 - protein, 24 aa, synthetic construct
GLFEAIEGFIENGWEGMIDGWYGC

SEQ ID NO: 203 - protein, 20 aa, synthetic construct
GLWRALWRLLRSLWRLLWRA

SEQ ID NO: 204 - protein, 15 aa, synthetic construct
SDLWEMMMVSLACQY

SEQ ID NO: 205 - protein, 12 aa, synthetic construct
TGNYKALHPHNG

SEQ ID NO: 206 - protein, 5 aa, synthetic construct
VPMLK

SEQ ID NO: 207 - protein, 14 aa, synthetic construct
RRRRRRGGRRRRRG

SEQ ID NO: 208 - protein, 19 aa, synthetic construct
KVKVKVKVDPPTKVKVKVK

SEQ ID NO: 209 - protein, 13 aa, synthetic construct
LAGRRRRRRRRRK

SEQ ID NO: 210 - protein, 15 aa, synthetic construct
RLWMRWYSPRTRAYG

SEQ ID NO: 211 - protein, 36 aa, synthetic construct
KKKKKKKKKKKKKKKKLRVRLASHLRKLRKRLLRDA

SEQ ID NO: 212 - protein, 19 aa, synthetic construct
TFFYGGSRGKRNNFKTEEY

SEQ ID NO: 213 - protein, 23 aa, synthetic construct
EEEEEEEEPLGLAGRRRRRRRRN

SEQ ID NO: 214 - protein, 23 aa, synthetic construct
KAFAKLAARLYRKALARQLGVAA

SEQ ID NO: 215 - protein, 24 aa, synthetic construct
LGTYTQDFNKFHTFPQTAIGVGAP

SEQ ID NO: 216 - protein, 34 aa, synthetic construct
DAATATRGRSAASRPTQRPRAPARSASRPRRPVE

SEQ ID NO: 217 - protein, 27 aa, synthetic construct
GALFLGFLGAAGSTMGAWSQPKSKRKV

SEQ ID NO: 218 - protein, 16 aa, synthetic construct
KPKRKRRKKKGHGWSR

SEQ ID NO: 219 - protein, 7 aa, synthetic construct
SGTQEEY

SEQ ID NO: 220 - protein, 13 aa, synthetic construct
RRRRRGGRRRRRG

SEQ ID NO: 221 - protein, 18 aa, synthetic construct
KWKLFKKIGAVLKVLTTG

SEQ ID NO: 222 - protein, 11 aa, synthetic construct
YARAAARQARA

SEQ ID NO: 223 - protein, 68 aa, synthetic construct
MKYFTLALTLLFLLLINPCKDMNFAWAESSEKVERASPQQAKYCYEQCNVNKVPFDQCYQMCSPLERS

SEQ ID NO: 224 - protein, 33 aa, Scorpio palmatus
GDCLPHLKLCKENKDCCSKKCKRRGTNIEKRCR

SEQ ID NO: 225 - protein, 33 aa, Pandinus imperator
GDCLPHLKRCKADNDCCGKKCKRRGTNAEKRCR

SEQ ID NO: 226 - protein, 35 aa, Hadrurus gertschi
SEKDCIKHLQRCRENKDCCSKKCSRRGTNPEKRCR

SEQ ID NO: 227 - protein, 33 aa, synthetic construct
GDCLPHLKLCKADKDCCSKKCKRRGTNPEKRCR

SEQ ID NO: 228 - protein, 33 aa, synthetic construct
GDCLPHLKRCKENNDCCSKKCKRRGTNPEKRCR

SEQ ID NO: 229 - protein, 33 aa, synthetic construct
GDCLPHLKRCKENNDCCSKKCKRRGANPEKRCR

SEQ ID NO: 230 - protein, 43 aa, synthetic construct
CKYKFENWGACDGGTGTKVRQGTLKKARYNAQCQETIRVTKPC

SEQ ID NO: 231 - protein, 34 aa, Momordica cochinchinensis
SGSDGGVCPKILKKCRRDSDCPGACICRGNGYCG

SEQ ID NO: 232 - protein, 36 aa, Leiurus quinquestriatus
MCMPCFTTDHQMARKCDDCCGGKGRGKCYGPQCLCR

SEQ ID NO: 233 - protein, 6 aa, synthetic construct
MLICLF

SEQ ID NO: 234 - protein, 4 aa, synthetic construct
GGGS

SEQ ID NO: 235 - protein, 20 aa, synthetic construct
  VARIANT 1..10: note = This region may encompass 1-10 residues
  VARIANT 11..20: note = This region may encompass 1-10 residues
GGGGGGGGGGSSSSSSSSSS

SEQ ID NO: 236 - protein, 20 aa, synthetic construct
  VARIANT 1..20: note = This sequence may encompass 1-10 GS repeating units
GSGSGSGSGSGSGSGSGSGS

SEQ ID NO: 237 - protein, 5 aa, synthetic construct
GGSSG

SEQ ID NO: 238 - protein, 5 aa, synthetic construct
GGGGG

SEQ ID NO: 239 - protein, 8 aa, synthetic construct
GSGSGSGS

SEQ ID NO: 240 - protein, 4 aa, synthetic construct
GSGG

SEQ ID NO: 241 - protein, 5 aa, synthetic construct
GGGGS

SEQ ID NO: 242 - skipped

SEQ ID NO: 243 - protein, 12 aa, synthetic construct
GGGSGGGSGGGS

SEQ ID NO: 244 - protein, 12 aa, synthetic construct
KKYKPYVPVTTN

SEQ ID NO: 245 - protein, 10 aa, synthetic construct
EPKSSDKTHT

SEQ ID NO: 246 - protein, 11 aa, synthetic construct
GGGSGGSGGGS

SEQ ID NO: 247-251 - skipped (5 entries)

SEQ ID NO: 252 - protein, 4 aa, synthetic construct
GSSS

SEQ ID NO: 253 - protein, 5 aa, synthetic construct
GSSSS

SEQ ID NO: 254 - protein, 40 aa, synthetic construct
  VARIANT 1..40: note = This sequence may encompass 1-10 GSSS repeating units
GSSSGSSSGSSSGSSSGSSSGSSSGSSSGSSSGSSSGSSS

SEQ ID NO: 255-259 - skipped (5 entries)

SEQ ID NO: 260 - protein, 4 aa, synthetic construct
GVAG

SEQ ID NO: 261 - protein, 5 aa, synthetic construct
  SITE 3: note = citrulline
GVXGG

SEQ ID NO: 262-268 - skipped (7 entries)

SEQ ID NO: 269 - protein, 5 aa, synthetic construct
  SITE 1: note = 6-azidohexanoyl
XGVAG

SEQ ID NO: 270 - protein, 6 aa, synthetic construct
  SITE 1: note = 6-azidohexanoyl
  SITE 4: note = citrulline
XGVXGG

SEQ ID NO: 271-283 - skipped (13 entries)

SEQ ID NO: 284 - protein, 8 aa, synthetic construct
GPQGIAGQ

SEQ ID NO: 285 - protein, 8 aa, synthetic construct
GPQGIFGQ

SEQ ID NO: 286 - protein, 8 aa, synthetic construct
GPQGIWGQ

SEQ ID NO: 287 - protein, 8 aa, synthetic construct
GPQGILGQ

SEQ ID NO: 288 - protein, 8 aa, synthetic construct
GPQGIRGQ

SEQ ID NO: 289 - protein, 8 aa, synthetic construct
GPLGIAGQ

SEQ ID NO: 290 - protein, 8 aa, synthetic construct
GPMGIAGQ

SEQ ID NO: 291 - protein, 8 aa, synthetic construct
GPYGIAGQ

SEQ ID NO: 292 - protein, 6 aa, synthetic construct
GSVAGS

SEQ ID NO: 293 - protein, 12 aa, synthetic construct
GGGGSVAGGGGS

SEQ ID NO: 294 - protein, 22 aa, synthetic construct
GGGGSGGGGSVAGGGGSGGGGS

SEQ ID NO: 295 - protein, 26 aa, synthetic construct
GGGGSGGGGSPLGLAGGGGGSGGGGS

SEQ ID NO: 296 - protein, 26 aa, synthetic construct
AEAAAKEAAAKAVAAEAAAKEAAAKA

SEQ ID NO: 297 - DNA, 20 bp, synthetic construct
  source 1..20: mol_type = other DNA
tgtacttccagggaggatcc

SEQ ID NO: 298 - DNA, 17 bp, synthetic construct
  source 1..17: mol_type = other DNA
aatggtgatgagcggcc

SEQ ID NO: 299 - skipped

SEQ ID NO: 300 - protein, 7 aa, synthetic construct
PKKKRRV

SEQ ID NO: 301 - protein, 16 aa, synthetic construct
KRPAATKKAGQAKKKK

SEQ ID NO: 302 - protein, 20 aa, synthetic construct
  VARIANT 1..10: note = This region may encompass 1-10 residues
  VARIANT 11..20: note = This region may encompass 1-10 residues
KKKKKKKKKKRRRRRRRRRR

SEQ ID NO: 303 - protein, 4 aa, synthetic construct
KKRR

SEQ ID NO: 304 - protein, 5 aa, synthetic construct
KKKRR

SEQ ID NO: 305 - protein, 4 aa, synthetic construct
KKKK

SEQ ID NO: 306 - protein, 51 aa, synthetic construct
  VARIANT 8: note = serine, threonine, aspartic acid, or asparagine
  VARIANT 11: note = alanine, methionine, isoleucine, or leucine
  VARIANT 14: note = aspartic acid, glutamic acid, histidine, lysine, arginine, asparagine, glutamine, serine, or threonine
  VARIANT 17: note = alanine, methionine, isoleucine, or leucine
  VARIANT 18: note = aspartic acid, glutamic acid, asparagine, glutamine, serine, or threonine
  VARIANT 21: note = aspartic acid, glutamic acid, asparagine, glutamine, serine, or threonine
  VARIANT 38: note = alanine, methionine, isoleucine, or leucine
  VARIANT 41: note = alanine, methionine, isoleucine, or leucine
  VARIANT 42: note = alanine, methionine, isoleucine, or leucine
  VARIANT 45: note = alanine, methionine, isoleucine, or leucine
  VARIANT 46: note = aspartic acid, glutamic acid, asparagine, glutamine, serine, or threonine
  VARIANT 47: note = histidine, lysine, arginine, asparagine, glutamine, serine, or threonine
  VARIANT 49: note = histidine, lysine, arginine, asparagine, glutamine, serine, or threonine
  VARIANT 50: note = serine, threonine, aspartic acid, or asparagine
  VARIANT 51: note = aspartic acid, glutamic acid, histidine, lysine, arginine, asparagine, glutamine, serine, or threonine
GSREGCAXRCXKYXDEXXKCXARMMSMSNTEEDCEQEXEDXXYCXXXCXXX

SEQ ID NO: 307 - protein, 51 aa, synthetic construct
  VARIANT 5: note = Any amino acid
  VARIANT 7: note = Any amino acid
  VARIANT 8: note = Any amino acid
  VARIANT 11: note = Any amino acid
  VARIANT 14: note = Any amino acid
  VARIANT 17: note = Any amino acid
  VARIANT 18: note = Any amino acid
  VARIANT 21: note = Any amino acid
GSREXCXXRCXKYXDEXXKCXARMMSMSNTEEDCEQELEDLLYCLDHCHSQ

SEQ ID NO: 308 - protein, 51 aa, synthetic construct
  VARIANT 38: note = Any amino acid
  VARIANT 41: note = Any amino acid
  VARIANT 42: note = Any amino acid
  VARIANT 45: note = Any amino acid
  VARIANT 46: note = Any amino acid
  VARIANT 47: note = Any amino acid
  VARIANT 49: note = Any amino acid
  VARIANT 50: note = Any amino acid
  VARIANT 51: note = Any amino acid
GSREGCASRCMKYNDELEKCEARMMSMSNTEEDCEQEXEDXXYCXXXCXXX

SEQ ID NO: 309 - protein, 51 aa, synthetic construct
  VARIANT 5: note = Any amino acid
  VARIANT 7: note = Any amino acid
  VARIANT 8: note = Any amino acid
  VARIANT 11: note = Any amino acid
  VARIANT 14: note = Any amino acid
  VARIANT 17: note = Any amino acid
  VARIANT 18: note = Any amino acid
  VARIANT 21: note = Any amino acid
  VARIANT 38: note = Any amino acid
  VARIANT 41: note = Any amino acid
  VARIANT 42: note = Any amino acid
  VARIANT 45: note = Any amino acid
  VARIANT 46: note = Any amino acid
  VARIANT 47: note = Any amino acid
  VARIANT 49: note = Any amino acid
  VARIANT 50: note = Any amino acid
  VARIANT 51: note = Any amino acid
GSREXCXXRCXKYXDEXXKCXARMMSMSNTEEDCEQEXEDXXYCXXXCXXX

SEQ ID NO: 310 - protein, 51 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 6: note = Any amino acid
  VARIANT 9: note = Any amino acid
  VARIANT 10: note = Any amino acid
  VARIANT 12: note = Any amino acid
  VARIANT 13: note = Any amino acid
  VARIANT 15: note = Any amino acid
  VARIANT 16: note = Any amino acid
  VARIANT 19: note = Any amino acid
  VARIANT 20: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 35: note = Any amino acid
  VARIANT 36: note = Any amino acid
  VARIANT 37: note = Any amino acid
  VARIANT 38: note = Any amino acid
  VARIANT 39: note = Any amino acid
  VARIANT 40: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 46: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
XXXXGXASXXMXXNXXLEXXEXXXXXXXXXXXXXXXXXXXXXXXXXXXXXX

SEQ ID NO: 311 - protein, 51 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 35: note = Any amino acid
  VARIANT 36: note = Any amino acid
  VARIANT 37: note = Any amino acid
  VARIANT 39: note = Any amino acid
  VARIANT 40: note = Any amino acid
  VARIANT 43: note = Any amino acid
  VARIANT 44: note = Any amino acid
  VARIANT 48: note = Any amino acid
XXXXXXXXXXXXXXXXXXXXXXXXXXXXXXXXXXXXXLXXLLXXLDHXHSQ

SEQ ID NO: 312 - protein, 51 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 6: note = Any amino acid
  VARIANT 9: note = Any amino acid
  VARIANT 10: note = Any amino acid
  VARIANT 12: note = Any amino acid
  VARIANT 13: note = Any amino acid
  VARIANT 15: note = Any amino acid
  VARIANT 16: note = Any amino acid
  VARIANT 19: note = Any amino acid
  VARIANT 20: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 35: note = Any amino acid
  VARIANT 36: note = Any amino acid
  VARIANT 37: note = Any amino acid
  VARIANT 39: note = Any amino acid
  VARIANT 40: note = Any amino acid
  VARIANT 43: note = Any amino acid
  VARIANT 44: note = Any amino acid
  VARIANT 48: note = Any amino acid
XXXXGXASXXMXXNXXLEXXEXXXXXXXXXXXXXXXXLXXLLXXLDHXHSQ

SEQ ID NO: 313 - protein, 51 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 6: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 15: note = Any amino acid
  VARIANT 16: note = Any amino acid
  VARIANT 19: note = Any amino acid
  VARIANT 20: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 35: note = Any amino acid
  VARIANT 36: note = Any amino acid
  VARIANT 37: note = Any amino acid
  VARIANT 39: note = Any amino acid
  VARIANT 40: note = Any amino acid
  VARIANT 41: note = Any amino acid
  VARIANT 43: note = Any amino acid
  VARIANT 44: note = Any amino acid
  VARIANT 48: note = Any amino acid
  VARIANT 49: note = Any amino acid
XXXXGXASXXXXXNXXLEXXEXXXXXXXXXXXXXXXXLXXXLXXLDHXXSQ

SEQ ID NO: 314 - protein, 51 aa, synthetic construct
  VARIANT 1: note = Any amino acid
  VARIANT 2: note = Any amino acid
  VARIANT 5: note = Any amino acid
  VARIANT 6: note = Any amino acid
  VARIANT 7: note = Any amino acid
  VARIANT 8: note = Any amino acid
  VARIANT 10: note = Any amino acid
  VARIANT 11: note = Any amino acid
  VARIANT 13: note = Any amino acid
  VARIANT 14: note = Any amino acid
  VARIANT 17: note = Any amino acid
  VARIANT 18: note = Any amino acid
  VARIANT 20: note = Any amino acid
  VARIANT 21: note = Any amino acid
  VARIANT 22: note = Any amino acid
  VARIANT 24: note = Any amino acid
  VARIANT 25: note = Any amino acid
  VARIANT 27: note = Any amino acid
  VARIANT 34: note = Any amino acid
  VARIANT 38: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 46: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
XXREXXXXRXXKXXDEXXKXXXRXXSXSNTEEDXEQEXEDXXXXXXXXXXX

SEQ ID NO: 315 - protein, 51 aa, synthetic construct
  VARIANT 3: note = aspartic acid, glutamic acid, histidine, lysine, arginine, asparagine, glutamine, serine, or threonine
  VARIANT 4: note = aspartic acid, glutamic acid, histidine, lysine, arginine, asparagine, glutamine, serine, or threonine
  VARIANT 9: note = aspartic acid, glutamic acid, histidine, lysine, arginine, asparagine, glutamine, serine, or threonine
  VARIANT 12: note = aspartic acid, glutamic acid, histidine, lysine, arginine, asparagine, glutamine, serine, or threonine
  VARIANT 15: note = aspartic acid, glutamic acid, histidine, lysine, arginine, asparagine, glutamine, serine, or threonine
  VARIANT 16: note = aspartic acid, glutamic acid, histidine, lysine, arginine, asparagine, glutamine, serine, or threonine
  VARIANT 19: note = aspartic acid, glutamic acid, histidine, lysine, arginine, asparagine, glutamine, serine, or threonine
  VARIANT 23: note = aspartic acid, glutamic acid, histidine, lysine, arginine, asparagine, glutamine, serine, or threonine
  VARIANT 26: note = aspartic acid, glutamic acid, histidine, lysine, arginine, asparagine, glutamine, serine, or threonine
  VARIANT 28: note = aspartic acid, glutamic acid, histidine, lysine, arginine, asparagine, glutamine, serine, or threonine
  VARIANT 29: note = aspartic acid, glutamic acid, histidine, lysine, arginine, asparagine, glutamine, serine, or threonine
  VARIANT 30: note = aspartic acid, glutamic acid, histidine, lysine, arginine, asparagine, glutamine, serine, or threonine
  VARIANT 31: note = aspartic acid, glutamic acid, histidine, lysine, arginine, asparagine, glutamine, serine, or threonine
  VARIANT 32: note = aspartic acid, glutamic acid, histidine, lysine, arginine, asparagine, glutamine, serine, or threonine
  VARIANT 33: note = aspartic acid, glutamic acid, histidine, lysine, arginine, asparagine, glutamine, serine, or threonine
  VARIANT 35: note = aspartic acid, glutamic acid, histidine, lysine, arginine, asparagine, glutamine, serine, or threonine
  VARIANT 36: note = aspartic acid, glutamic acid, histidine, lysine, arginine, asparagine, glutamine, serine, or threonine
  VARIANT 37: note = aspartic acid, glutamic acid, histidine, lysine, arginine, asparagine, glutamine, serine, or threonine
  VARIANT 39: note = aspartic acid, glutamic acid, histidine, lysine, arginine, asparagine, glutamine, serine, or threonine
  VARIANT 40: note = aspartic acid, glutamic acid, histidine, lysine, arginine, asparagine, glutamine, serine, or threonine
GSXXGCASXCMXYNXXLEXCEAXMMXMXXXXXXCXXXLXXLLYCLDHCHSQ

SEQ ID NO: 316 - protein, 51 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 36: note = Any amino acid
  VARIANT 37: note = Any amino acid
  VARIANT 38: note = Any amino acid
  VARIANT 40: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 46: note = Any amino acid
  VARIANT 47: note = Any amino acid
  VARIANT 48: note = Any amino acid
  VARIANT 50: note = Any amino acid
  VARIANT 51: note = Any amino acid
XXXXXXXXXXXXXXDXXXXXXXXXXXXXXXXXXXEXXXEXXXXXXXXXHXX

SEQ ID NO: 317 - protein, 51 aa, synthetic construct
  VARIANT 15: note = aspartic acid, glutamic acid, histidine, lysine, arginine, asparagine, glutamine, serine, or threonine
  VARIANT 35: note = aspartic acid, glutamic acid, histidine, lysine, arginine, asparagine, glutamine, serine, or threonine
  VARIANT 39: note = aspartic acid, glutamic acid, histidine, lysine, arginine, asparagine, glutamine, serine, or threonine
  VARIANT 49: note = aspartic acid, glutamic acid, histidine, lysine, arginine, asparagine, glutamine, serine, or threonine
GSREGCASRCMKYNXELEKCEARMMSMSNTEEDCXQELXDLLYCLDHCXSQ

SEQ ID NO: 318 - protein, 51 aa, synthetic construct; identical to SEQ ID NO: 317
  VARIANT 15: note = alanine, methionine, isoleucine, leucine, valine, phenylalanine, tryptophan, or tyrosine
  VARIANT 35: note = alanine, methionine, isoleucine, leucine, valine, phenylalanine, tryptophan, or tyrosine
  VARIANT 39: note = alanine, methionine, isoleucine, leucine, valine, phenylalanine, tryptophan, or tyrosine
  VARIANT 49: note = alanine, methionine, isoleucine, leucine, valine, phenylalanine, tryptophan, or tyrosine

SEQ ID NO: 319 - skipped

SEQ ID NO: 320 - protein, 51 aa, synthetic construct
  VARIANT 11: note = alanine, methionine, isoleucine, leucine, valine, phenylalanine, tryptophan, or tyrosine
  VARIANT 25: note = alanine, methionine, isoleucine, leucine, valine, phenylalanine, tryptophan, or tyrosine
  VARIANT 27: note = alanine, methionine, isoleucine, leucine, valine, phenylalanine, tryptophan, or tyrosine
GSREGCASRCXKYNDELEKCEARMXSXSNTEEDCEQELEDLLYCLDHCHSQ

SEQ ID NO: 321 - protein, 51 aa, synthetic construct; identical to SEQ ID NO: 320
  VARIANT 11: note = aspartic acid, glutamic acid, histidine, lysine, arginine, asparagine, glutamine, serine, or threonine
  VARIANT 25: note = aspartic acid, glutamic acid, histidine, lysine, arginine, asparagine, glutamine, serine, or threonine
  VARIANT 27: note = aspartic acid, glutamic acid, histidine, lysine, arginine, asparagine, glutamine, serine, or threonine

SEQ ID NO: 322 - skipped

SEQ ID NO: 323 - protein, 51 aa, synthetic construct
  VARIANT 45: note = alanine, methionine, isoleucine, leucine, or valine
GSREGCASRCMKYNDELEKCEARMMSMSNTEEDCEQELEDLLYCXDHCHSQ

SEQ ID NO: 324 - protein, 51 aa, synthetic construct
  VARIANT 12: note = lysine or arginine
  VARIANT 19: note = lysine or arginine
GSREGCASRCMXYNDELEXCEARMMSMSNTEEDCEQELEDLLYCLDHCHSQ

SEQ ID NO: 325 - protein, 49 aa, synthetic construct
  VARIANT 6: note = serine, threonine, aspartic acid, or asparagine
  VARIANT 9: note = alanine, methionine, isoleucine, leucine, or valine
  VARIANT 12: note = aspartic acid, glutamic acid, histidine, lysine, arginine, asparagine, glutamine, serine, or threonine
  VARIANT 15: note = alanine, methionine, isoleucine, leucine, or valine
  VARIANT 16: note = aspartic acid, glutamic acid, asparagine, glutamine, serine, or threonine
  VARIANT 19: note = aspartic acid, glutamic acid, asparagine, glutamine, serine, or threonine
  VARIANT 36: note = alanine, methionine, isoleucine, leucine, or valine
  VARIANT 39: note = alanine, methionine, isoleucine, leucine, or valine
  VARIANT 40: note = alanine, methionine, isoleucine, leucine, or valine
  VARIANT 43: note = alanine, methionine, isoleucine, leucine, or valine
  VARIANT 44: note = aspartic acid, glutamic acid, asparagine, glutamine, serine, or threonine
  VARIANT 45: note = histidine, lysine, arginine, asparagine, glutamine, serine, or threonine
  VARIANT 47: note = histidine, lysine, arginine, asparagine, glutamine, serine, or threonine
  VARIANT 48: note = serine, threonine, aspartic acid, or asparagine
  VARIANT 49: note = aspartic acid, glutamic acid, histidine, lysine, arginine, asparagine, glutamine, serine, or threonine
REGCAXRCXKYXDEXXKCXARMMSMSNTEEDCEQEXEDXXYCXXXCXXX

SEQ ID NO: 326 - protein, 49 aa, synthetic construct
  VARIANT 3: note = Any amino acid
  VARIANT 5: note = Any amino acid
  VARIANT 6: note = Any amino acid
  VARIANT 9: note = Any amino acid
  VARIANT 12: note = Any amino acid
  VARIANT 15: note = Any amino acid
  VARIANT 16: note = Any amino acid
  VARIANT 19: note = Any amino acid
REXCXXRCXKYXDEXXKCXARMMSMSNTEEDCEQELEDLLYCLDHCHSQ

SEQ ID NO: 327 - protein, 49 aa, synthetic construct
  VARIANT 36: note = Any amino acid
  VARIANT 39: note = Any amino acid
  VARIANT 40: note = Any amino acid
  VARIANT 43: note = Any amino acid
  VARIANT 44: note = Any amino acid
  VARIANT 45: note = Any amino acid
  VARIANT 47: note = Any amino acid
  VARIANT 48: note = Any amino acid
  VARIANT 49: note = Any amino acid
REGCASRCMKYNDELEKCEARMMSMSNTEEDCEQEXEDXXYCXXXCXXX

SEQ ID NO: 328 - protein, 49 aa, synthetic construct
  VARIANT 3: note = Any amino acid
  VARIANT 5: note = Any amino acid
  VARIANT 6: note = Any amino acid
  VARIANT 9: note = Any amino acid
  VARIANT 12: note = Any amino acid
  VARIANT 15: note = Any amino acid
  VARIANT 16: note = Any amino acid
  VARIANT 19: note = Any amino acid
  VARIANT 36: note = Any amino acid
  VARIANT 39: note = Any amino acid
  VARIANT 40: note = Any amino acid
  VARIANT 43: note = Any amino acid
  VARIANT 44: note = Any amino acid
  VARIANT 45: note = Any amino acid
  VARIANT 47: note = Any amino acid
  VARIANT 48: note = Any amino acid
  VARIANT 49: note = Any amino acid
REXCXXRCXKYXDEXXKCXARMMSMSNTEEDCEQEXEDXXYCXXXCXXX

SEQ ID NO: 329 - protein, 49 aa, synthetic construct
  VARIANT 1: note = aspartic acid, glutamic acid, histidine, lysine, arginine, asparagine, glutamine, serine, or threonine
  VARIANT 2: note = aspartic acid, glutamic acid, histidine, lysine, arginine, asparagine, glutamine, serine, or threonine
  VARIANT 7: note = aspartic acid, glutamic acid, histidine, lysine, arginine, asparagine, glutamine, serine, or threonine
  VARIANT 10: note = aspartic acid, glutamic acid, histidine, lysine, arginine, asparagine, glutamine, serine, or threonine
  VARIANT 13: note = aspartic acid, glutamic acid, histidine, lysine, arginine, asparagine, glutamine, serine, or threonine
  VARIANT 14: note = aspartic acid, glutamic acid, histidine, lysine, arginine, asparagine, glutamine, serine, or threonine
  VARIANT 17: note = aspartic acid, glutamic acid, histidine, lysine, arginine, asparagine, glutamine, serine, or threonine
  VARIANT 21: note = aspartic acid, glutamic acid, histidine, lysine, arginine, asparagine, glutamine, serine, or threonine
  VARIANT 24: note = aspartic acid, glutamic acid, histidine, lysine, arginine, asparagine, glutamine, serine, or threonine
  VARIANT 26: note = aspartic acid, glutamic acid, histidine, lysine, arginine, asparagine, glutamine, serine, or threonine
  VARIANT 27: note = aspartic acid, glutamic acid, histidine, lysine, arginine, asparagine, glutamine, serine, or threonine
  VARIANT 28: note = aspartic acid, glutamic acid, histidine, lysine, arginine, asparagine, glutamine, serine, or threonine
  VARIANT 29: note = aspartic acid, glutamic acid, histidine, lysine, arginine, asparagine, glutamine, serine, or threonine
  VARIANT 30: note = aspartic acid, glutamic acid, histidine, lysine, arginine, asparagine, glutamine, serine, or threonine
  VARIANT 31: note = aspartic acid, glutamic acid, histidine, lysine, arginine, asparagine, glutamine, serine, or threonine
  VARIANT 33: note = aspartic acid, glutamic acid, histidine, lysine, arginine, asparagine, glutamine, serine, or threonine
  VARIANT 34: note = aspartic acid, glutamic acid, histidine, lysine, arginine, asparagine, glutamine, serine, or threonine
  VARIANT 35: note = aspartic acid, glutamic acid, histidine, lysine, arginine, asparagine, glutamine, serine, or threonine
  VARIANT 37: note = aspartic acid, glutamic acid, histidine, lysine, arginine, asparagine, glutamine, serine, or threonine
  VARIANT 38: note = aspartic acid, glutamic acid, histidine, lysine, arginine, asparagine, glutamine, serine, or threonine
XXGCASXCMXYNXXLEXCEAXMMXMXXXXXXCXXXLXXLLYCLDHCHSQ

SEQ ID NO: 330 - protein, 49 aa, synthetic construct
  VARIANT 13: note = aspartic acid, glutamic acid, histidine, lysine, arginine, asparagine, glutamine, serine, or threonine
  VARIANT 33: note = aspartic acid, glutamic acid, histidine, lysine, arginine, asparagine, glutamine, serine, or threonine
  VARIANT 37: note = aspartic acid, glutamic acid, histidine, lysine, arginine, asparagine, glutamine, serine, or threonine
  VARIANT 47: note = aspartic acid, glutamic acid, histidine, lysine, arginine, asparagine, glutamine, serine, or threonine
REGCASRCMKYNXELEKCEARMMSMSNTEEDCXQELXDLLYCLDHCXSQ

SEQ ID NO: 331 - protein, 49 aa, synthetic construct; identical to SEQ ID NO: 330
  VARIANT 13: note = alanine, methionine, isoleucine, leucine, valine, phenylalanine, tryptophan, or tyrosine
  VARIANT 33: note = alanine, methionine, isoleucine, leucine, valine, phenylalanine, tryptophan, or tyrosine
  VARIANT 37: note = alanine, methionine, isoleucine, leucine, valine, phenylalanine, tryptophan, or tyrosine
  VARIANT 47: note = alanine, methionine, isoleucine, leucine, valine, phenylalanine, tryptophan, or tyrosine

SEQ ID NO: 332 - protein, 49 aa, synthetic construct
  VARIANT 9: note = alanine, methionine, isoleucine, leucine, valine, phenylalanine, tryptophan, or tyrosine
  VARIANT 23: note = alanine, methionine, isoleucine, leucine, valine, phenylalanine, tryptophan, or tyrosine
  VARIANT 25: note = alanine, methionine, isoleucine, leucine, valine, phenylalanine, tryptophan, or tyrosine
REGCASRCXKYNDELEKCEARMXSXSNTEEDCEQELEDLLYCLDHCHSQ

SEQ ID NO: 333 - protein, 49 aa, synthetic construct; identical to SEQ ID NO: 332
  VARIANT 9: note = aspartic acid, glutamic acid, histidine, lysine, arginine, asparagine, glutamine, serine, or threonine
  VARIANT 23: note = aspartic acid, glutamic acid, histidine, lysine, arginine, asparagine, glutamine, serine, or threonine
  VARIANT 25: note = aspartic acid, glutamic acid, histidine, lysine, arginine, asparagine, glutamine, serine, or threonine

SEQ ID NO: 334 - protein, 49 aa, synthetic construct
  VARIANT 43: note = alanine, methionine, isoleucine, leucine, or valine
REGCASRCMKYNDELEKCEARMMSMSNTEEDCEQELEDLLYCXDHCHSQ

SEQ ID NO: 335 - protein, 49 aa, synthetic construct
  VARIANT 10: note = lysine or arginine
  VARIANT 17: note = lysine or arginine
REGCASRCMXYNDELEXCEARMMSMSNTEEDCEQELEDLLYCLDHCHSQ

SEQ ID NO: 336 - protein, 6 aa, synthetic construct
HHHHHH

SEQ ID NO: 337 - protein, 8 aa, synthetic construct
DYKDDDDK

SEQ ID NO: 338 - protein, 5 aa, synthetic construct
DYKDE

SEQ ID NO: 339 - protein, 9 aa, synthetic construct
YPYDVPDYA

SEQ ID NO: 340 - protein, 9 aa, synthetic construct
DVEAWLGAR

SEQ ID NO: 341 - protein, 13 aa, Homo sapiens
ELYENKPRRPYIL

SEQ ID NO: 342 - protein, 221 aa, synthetic construct
METDTLLLWVLLLWVPGSTGDYKDEHHHHHHGGSQDSTSDLIPAPPLSKVPLQQNFQDNQFQGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDGGGSENLYFQ

SEQ ID NO: 343 - protein, 891 aa, synthetic construct
METDTLLLWVLLLWVPGSTGDYKDEHHHHHHGGSQDSTSDLIPAPPLSKVPLQQNFQDNQFQGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDGGGSENLYFQGSRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEFGGGSHHHHHHGGGSLNDIFEAQKIEWHE

SEQ ID NO: 344 - protein, 727 aa, synthetic construct
MRLAVGALLVCAVLGLCLADYKDEHHHHHHGLNDIFEAQKIEWHEGGGSVPDKTVKWCAVSEHENTKCISFRDHMKTVLPPDGPRLACVKKTSYPDCIKAISASEADAMTLDGGWVYDAGLTPNNLKPVAAEFYGSVEHPQTYYYAVAVVKKGTDFQLNQLEGKKSCHTGLGRSAGWVIPIGLLFCKLSEPRSPLEKAVSSFFSGSCVPCADPVAFPKLCQLCPGCGCSSTQPFFGYVGAFKCLKDGGGDVAFVKHTTIFEVLPEKADRDQYELLCLDNTRKPVDQYEDCYLARIPSHAVVARKNNGKEDLIWEILKVAQEHFGKGKSKDFQLFSSPLGKDLLFKDSAFGLLRVPPRMDYRLYLGHNYVTAIRNQQEGVCPEGSIDNSPVKWCALSHLERTKCDEWSIISEGKIECESAETTEDCIEKIVNGEADAMTLDGGHAYIAGQCGLVPVMAEYYESSNCAIPSQQGIFPKGYYAVAVVKASDTSITWNNLKGKKSCHTGVDRTAGWNIPMGMLYNRINHCKFDEFFSQGCAPGYEKNSTLCDLCIGPLKCAPNNKEEYNGYTGAFRCLVEKGDVAFVKHQTVLDNTEGKNPAEWAKNLKQEDFELLCPDGTRKPVKDFASCHLAQAPNHVVVSRKEKAARVKAVLTSQETLFGGSDCTGNFCLFKSTTKDLLFRDDTKCFVKLPEGTTPEKYLGAEYMQSVGNMRKCSTSRLLEACTFHKH

SEQ ID NO: 345 - protein, 743 aa, synthetic construct
MRLAVGALLVCAVLGLCLADYKDEHHHHHHGLNDIFEAQKIEWHEGGGSVPDKTVKWCAVSEHENTKCISFRDHMKTVLPPDGPRLACVKKTSYPDCIKAISASEADAMTLDGGWVYDAGLTPNNLKPVAAEFYGSVEHPQTYYYAVAVVKKGTDFQLNQLEGKKSCHTGLGRSAGWVIPIGLLFCKLSEPRSPLEKAVSSFFSGSCVPCADPVAFPKLCQLCPGCGCSSTQPFFGYVGAFKCLKDGGGDVAFVKHTTIFEVLPEKADRDQYELLCLDNTRKPVDQYEDCYLARIPSHAVVARKNNGKEDLIWEILKVAQEHFGKGKSKDFQLFSSPLGKDLLFKDSAFGLLRVPPRMDYRLYLGHNYVTAIRNQQEGVCPEGSIDNSPVKWCALSHLERTKCDEWSIISEGKIECESAETTEDCIEKIVNGEADAMTLDGGHAYIAGQCGLVPVMAEYYESSNCAIPSQQGIFPKGYYAVAVVKASDTSITWNNLKGKKSCHTGVDRTAGWNIPMGMLYNRINHCKFDEFFSQGCAPGYEKNSTLCDLCIGPLKCAPNNKEEYNGYTGAFRCLVEKGDVAFVKHQTVLDNTEGKNPAEWAKNLKQEDFELLCPDGTRKPVKDFASCHLAQAPNHVVVSRKEKAARVKAVLTSQETLFGGSDCTGNFCLFKSTTKDLLFRDDTKCFVKLPEGTTPEKYLGAEYMQSVGNMRKCSTSRLLEACTFHKHGSSELYENKPRRPYIL

SEQ ID NO: 346 - protein, 272 aa, synthetic construct
METDTLLLWVLLLWVPGSTGDYKDEHHHHHHGGSQDSTSDLIPAPPLSKVPLQQNFQDNQFQGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDGGGSENLYFQGSREGCASRCTKYNAELEKCEARVSSMSNTEETCVQELFDLLHCVDHCVSQ

SEQ ID NO: 347 - protein, 272 aa, synthetic construct
METDTLLLWVLLLWVPGSTGDYKDEHHHHHHGGSQDSTSDLIPAPPLSKVPLQQNFQDNQFQGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDGGGSENLYFQGSREGCASRCTKYNAELEKCEARVMSMSNTEEDCEQELEDLLHCLDHCHSQ

SEQ ID NO: 348 - protein, 272 aa, synthetic construct
METDTLLLWVLLLWVPGSTGDYKDEHHHHHHGGSQDSTSDLIPAPPLSKVPLQQNFQDNQFQGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDGGGSENLYFQGSREGCASRCMKYNDELEKCEARMMSMSNTEEDCEQELEDLLYCLDHCHSQ

SEQ ID NO: 349 - protein, 60 aa, Homo sapiens
MMDQARSAFSNLFGGEPLSYTRFSLARQVDGDNSHVEMKLAVDEEENADNNTKANVTKPK

SEQ ID NO: 350 - protein, 13 aa, synthetic construct
  SITE 8: note = N-methyl-arginine
  SITE 11: note = l-neo-tryptophan
  SITE 12: note = tert-leucine
ELYENKPRKPWLL

SEQ ID NO: 351 - protein, 13 aa, synthetic construct
  SITE 9: note = D-lysine
  SITE 11: note = l-neo-tryptophan
  SITE 12: note = tert-isoleucine
ELYENKPRKPWIL

SEQ ID NO: 352 - protein, 13 aa, synthetic construct
  SITE 9: note = D-ornithine
  SITE 11: note = neo-tryptophan
  SITE 12: note = tert-leucine
ELYENKPRXPWLL

SEQ ID NO: 353 - protein, 13 aa, synthetic construct
  SITE 8: note = N-methyl-arginine
  SITE 11: note = 3,1-naphthyl-D-alanine
  SITE 12: note = tert-leucine
ELYENKPRRPALL

SEQ ID NO: 354 - protein, 13 aa, synthetic construct
  SITE 8: note = lysine with CH2NH peptide bond isostere
  SITE 12: note = tert-leucine
  SITE 13: note = leucine ethyl ester
ELYENKPKKPYLL

SEQ ID NO: 355 - protein, 7 aa, synthetic construct
ELYENKP

SEQ ID NO: 356 - protein, 13 aa, synthetic construct
  VARIANT 8: note = lysine or arginine
  VARIANT 9: note = lysine or arginine
  VARIANT 11: note = tryptophan or tyrosine
  VARIANT 12: note = isoleucine or leucine
ELYENKPXXPXXL

SEQ ID NO: 357 - protein, 18 aa, synthetic construct
RLIEDICLPRWGCLWEDD

SEQ ID NO: 358 - protein, 38 aa, synthetic construct
GGGGSGGGGSRLIEDICLPRWGCLWEDDGGGGSGGGGS

SEQ ID NO: 359 - protein, 36 aa, synthetic construct
AAAISCVGSPECPPKCRAQGCKNGKCMNRKCKCYYC

SEQ ID NO: 360 - protein, 35 aa, synthetic construct
RSCIDTIPKSRCTAFQCKHSMKYRLSFCRKTCGTC

SEQ ID NO: 361 - protein, 41 aa, synthetic construct
GSEWSSRSCIDTIPKSRCTAFQCKHSMKYRLSFCRKTCGTC

SEQ ID NO: 362 - protein, 38 aa, synthetic construct
GSAAAISCVGSPECPPKCRAQGCKNGKCMNRKCKCYYC

SEQ ID NO: 363 - protein, 39 aa, synthetic construct
EWSSRSCIDTIPKSRCTAFQCKHSMKYRLSFCRKTCGTC

SEQ ID NO: 364 - RNA, 19 nt, synthetic construct
  source 1..19: mol_type = other RNA
tcccctgccaggtaagtat

SEQ ID NO: 365 - RNA, 17 nt, synthetic construct
  source 1..17: mol_type = other RNA
ccctgccaggtaagtat

SEQ ID NO: 366 - RNA, 15 nt, synthetic construct
  source 1..15: mol_type = other RNA
ctgccaggtaagtat

SEQ ID NO: 367 - RNA, 14 nt, synthetic construct
  source 1..14: mol_type = other RNA
tgccaggtaagtat

SEQ ID NO: 368 - RNA, 13 nt, synthetic construct
  source 1..13: mol_type = other RNA
gccaggtaagtat

SEQ ID NO: 369 - RNA, 12 nt, synthetic construct
  source 1..12: mol_type = other RNA
ccaggtaagtat

SEQ ID NO: 370 - RNA, 11 nt, synthetic construct
  source 1..11: mol_type = other RNA
caggtaagtat

SEQ ID NO: 371 - RNA, 10 nt, synthetic construct
  source 1..10: mol_type = other RNA
caggtaagta

SEQ ID NO: 372 - RNA, 22 nt, synthetic construct
  source 1..22: mol_type = other RNA
tcaacatcagtctgataagcta

SEQ ID NO: 373 - RNA, 21 nt, synthetic construct
  source 1..21: mol_type = other RNA
caacatcagtctgataagcta

SEQ ID NO: 374 - RNA, 20 nt, synthetic construct
  source 1..20: mol_type = other RNA
aacatcagtctgataagcta

SEQ ID NO: 375 - RNA, 19 nt, synthetic construct
  source 1..19: mol_type = other RNA
acatcagtctgataagcta

SEQ ID NO: 376 - RNA, 18 nt, synthetic construct
  source 1..18: mol_type = other RNA
catcagtctgataagcta

SEQ ID NO: 377 - RNA, 17 nt, synthetic construct
  source 1..17: mol_type = other RNA
atcagtctgataagcta

SEQ ID NO: 378 - RNA, 16 nt, synthetic construct
  source 1..16: mol_type = other RNA
tcagtctgataagcta

SEQ ID NO: 379 - RNA, 15 nt, synthetic construct
  source 1..15: mol_type = other RNA
cagtctgataagcta

SEQ ID NO: 380 - RNA, 33 nt, synthetic construct
  source 1..33: mol_type = other RNA
gccgtacagttccacaaagggccaggtaagtat

SEQ ID NO: 381 - DNA, 20 bp, synthetic construct
  source 1..20: mol_type = other DNA
tgacttgtcaagtcatcctt

SEQ ID NO: 382 - DNA, 20 bp, synthetic construct
  source 1..20: mol_type = other DNA
gcaggttaagtgattaacca

SEQ ID NO: 383 - DNA, 20 bp, synthetic construct
  source 1..20: mol_type = other DNA
tcctctccacaattattgac

SEQ ID NO: 384 - DNA, 20 bp, synthetic construct
  source 1..20: mol_type = other DNA
gacgtatttaggagaggaag

SEQ ID NO: 385 - DNA, 20 bp, synthetic construct
  source 1..20: mol_type = other DNA
ctgatactatgcatgtggag

SEQ ID NO: 386 - DNA, 20 bp, synthetic construct
  source 1..20: mol_type = other DNA
gccgggtcattattcttttt

SEQ ID NO: 387 - RNA, 21 nt, synthetic construct
  source 1..21: mol_type = other RNA
aatattgtgttgcttagtgaa

SEQ ID NO: 388 - RNA, 21 nt, synthetic construct
  source 1..21: mol_type = other RNA
aattcactaagcaacacaata

SEQ ID NO: 389 - RNA, 21 nt, synthetic construct
  source 1..21: mol_type = other RNA
aacatgtgtccatgtgaagat

SEQ ID NO: 390 - RNA, 21 nt, synthetic construct
  source 1..21: mol_type = other RNA
aaatcttcacatggacacatg

SEQ ID NO: 391 - RNA, 21 nt, synthetic construct
  source 1..21: mol_type = other RNA
aagttcagattgcaaggcagc

SEQ ID NO: 392 - RNA, 21 nt, synthetic construct
  source 1..21: mol_type = other RNA
aagctgccttgcaatctgaac

SEQ ID NO: 393 - RNA, 21 nt, synthetic construct
  source 1..21: mol_type = other RNA
aaccaagtacttcttgccacc

SEQ ID NO: 394 - RNA, 21 nt, synthetic construct
  source 1..21: mol_type = other RNA
aaggtggcaagaagtacttgg

SEQ ID NO: 395 - RNA, 22 nt, synthetic construct
  source 1..22: mol_type = other RNA
tagcttatcagactgatgttga

SEQ ID NO: 396 - DNA, 2858 bp, Homo sapiens
  source 1..2858: mol_type = genomic DNA
aggcagtgaggaaagaagacatgacacagccaacaagggtggcagcctggctctgaagtggaattatgtgcttcaaacaggttgaaagagggaaacagtcttttcctgcttccagacatgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagttccccaggtcacatcaaaagcattccaacatgacacatggaattctgggctaacagttgcttccatctctacagggcaccttacttctgacaacagaggagacattggacttttattgggaatgatcgtctttgctgttatgttgtcaattctttctttgattgggatatttaacagatcattccgaactgggattaaaagaaggatcttattgttaataccaaagtggctttatgaagatattcctaatatgaaaaacagcaatgttgtgaaaatgctacaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctgggccatatgataagcatatgtttcagttctaccaatcttgtttccagagtagtgacatttctgtgctcctaccatcaccatgtaagaattcccgggagctccatgcctttttaattttagccattcttctgcctcatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtaggtggatcacctgaggtcaggagttcgagtccagcctggccaatatgctgaaaccctgtctctactaaaattacaaaaattagccggccatggtggcaggtgcttgtaatcccagctacttgggaggctgaggcaggagaatcacttgaaccaggaaggcagaggttgcactgagctgagattgtgccactgcactccagcctgggcaacaagagcaaaactctgtctggaaaaaaaaaaaaaa

SEQ ID NO: 397 - DNA, 6590 bp, Homo sapiens
  source 1..6590: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 398 - DNA, 5639 bp, Homo sapiens
  source 1..5639: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 399 - DNA, 5465 bp, Homo sapiens
  source 1..5465: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 400 - DNA, 5372 bp, Homo sapiens
  source 1..5372: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 401 - DNA, 5552 bp, Homo sapiens
  source 1..5552: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 402 - DNA, 6644 bp, Homo sapiens
  source 1..6644: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 403 - DNA, 5459 bp, Homo sapiens
  source 1..5459: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 404 - DNA, 5546 bp, Homo sapiens
  source 1..5546: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 405 - DNA, 6815 bp, Homo sapiens
  source 1..6815: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 406 - DNA, 6524 bp, Homo sapiens
  source 1..6524: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 407 - DNA, 4139 bp, Homo sapiens
  source 1..4139: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 408 - DNA, 5525 bp, Homo sapiens
  source 1..5525: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 409 - DNA, 6881 bp, Homo sapiens
  source 1..6881: mol_type = genomic DNA
acccctcgccgcaccacacacagcgcgggcttctagcgctcggcaccggcgggccaggcgcgtcctgccttcatttatccagcagcttttcggaaaatgcatttgctgttcggagtttaatcagaagaggattcctgcctccgtccccggctccttcatcgtcccctctcccctgtctctctcctggggaggcgtgaagcggtcccgtggatagagattcatgcctgtgcccgcgcgtgtgtgcgcgcgtgtaaattgccgagaaggggaaaacatcacaggacttctgcgaataccggactgaaaattgtaattcatctgccgccgccgctgccttttttttttctcgagctcttgagatctccggttgggattcctgcggattgacatttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 410 - DNA, 1595 bp, Homo sapiens
  source 1..1595: mol_type = genomic DNA
acccctcgccgcaccacacacagcgcgggcttctagcgctcggcaccggcgggccaggcgcgtcctgccttcatttatccagcagcttttcggaaaatgcatttgctgttcggagtttaatcagaagaggattcctgcctccgtccccggctccttcatcgtcccctctcccctgtctctctcctggggaggcgtgaagcggtcccgtggatagagattcatgcctgtgcccgcgcgtgtgtgcgcgcgtgtaaattgccgagaaggggaaaacatcacaggacttctgcgaataccggactgaaaattgtaattcatctgccgccgccgctgccttttttttttctcgagctcttgagatctccggttgggattcctgcggattgacatttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctgaggccacaggtccgagatgcgggggttggagtgcgggtgggctcctggggcaatgggaggctgtggagccggcgaaataaaatcagagttgttgct

SEQ ID NO: 411 - DNA, 72 bp, synthetic construct
  source 1..72: mol_type = other DNA
tgtcgggtagcttatcagactgatgttgactgttgaatctcatggcaacaccagtcgatgggctgtctgaca

SEQ ID NO: 412 - DNA, 3661 bp, Homo sapiens
  source 1..3661: mol_type = genomic DNA
cttcagatagattatatctggagtgaagaatcctgccacctatgtatctggcatagtattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataa

SEQ ID NO: 413 - DNA, 895 bp, Homo sapiens
  source 1..895: mol_type = genomic DNA
gcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaa

SEQ ID NO: 414 - DNA, 9905 bp, Homo sapiens
  source 1..9905: mol_type = genomic DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 415 - DNA, 9770 bp, Homo sapiens
  source 1..9770: mol_type = genomic DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacagggccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 416 - DNA, 9104 bp, Homo sapiens
  source 1..9104: mol_type = genomic DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaaggtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 417 - DNA, 3983 bp, Homo sapiens
  source 1..3983: mol_type = genomic DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctggtgagtggcttgtctggaaacagtcctgctcctcaacctcctcgacccactcagcagcagccagtctccagtgtccaagccaggtgctccctccagcatctccagagggggaaacagtggcagatttgcagacacagtgaagggcgtaaggagcagataaacacatgaccgagcctgcacaagctctttgttgtgtctggttgtttgctgtacctctgttgtaagaatgaatctgcaaaatttctagcttatgaagcaaatcacggacatacacatctgtgtgtgtgagtgttcatgatgtgtgtacatctgtgtatgtgtgtgtgtgtatgtgtgtgtttgtgacagatttgatccctgttctctctgctggctctatcttgacctgtgaaacgtatatttaactaattaaatattagttaatattaataaattttaagctttatccaga

SEQ ID NO: 418 - DNA, 3848 bp, Homo sapiens
  source 1..3848: mol_type = genomic DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacagggccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctggtgagtggcttgtctggaaacagtcctgctcctcaacctcctcgacccactcagcagcagccagtctccagtgtccaagccaggtgctccctccagcatctccagagggggaaacagtggcagatttgcagacacagtgaagggcgtaaggagcagataaacacatgaccgagcctgcacaagctctttgttgtgtctggttgtttgctgtacctctgttgtaagaatgaatctgcaaaatttctagcttatgaagcaaatcacggacatacacatctgtgtgtgtgagtgttcatgatgtgtgtacatctgtgtatgtgtgtgtgtgtatgtgtgtgtttgtgacagatttgatccctgttctctctgctggctctatcttgacctgtgaaacgtatatttaactaattaaatattagttaatattaataaattttaagctttatccaga

SEQ ID NO: 419 - DNA, 2872 bp, Homo sapiens
  source 1..2872: mol_type = genomic DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggccaggaaatgagagtctcaaagccatgttattctgcctttttaaactatcatcctgtaatcaaagtaatgatggcagcgtgtcccaccagagcgggagcccagctgctcaggagtcatgcttaggatggatcccttctcttctgccgtcagagtttcagctgggttggggtggatgcagccacctccatgcctggccttctgcatctgtgatcatcacggcctcctcctgccactgagcctcatgccttcacgtgtctgttccccccgcttttcctttctgccacccctgcacgtgggccgccaggttcccaagagtatcctacccatttccttccttccactccctttgccagtgcctctcaccccaactagtagctaaccatcacccccaggactgacctcttcctcctcgctgccagatgattgttcaaagcacagaatttgtcagaaacctgcagggactccatgctgccagccttctccgtaattagcatggccccagtccatgcttctagccttggttccttctgcccctctgtttgaaattctagagccagctgtgggacaattatctgtgtcaaaagccagatgtgaaaacatctcaataacaaactggctgctttgttcaatgctagaacaacgcctgtcacagagtagaaactcaaaaatatttgctgagtgaatgaacaaatgaataaatgcataataaataattaaccaccaatccaa

SEQ ID NO: 420 - DNA, 2254 bp, Homo sapiens
  source 1..2254: mol_type = genomic DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggtcctaataaatcttcactgtctgactttagtctcccactaaaactgcatttcctttctacaatttcaatttctccctttgcttcaaataaagtcctgacactattcatttga

SEQ ID NO: 421 - DNA, 1575 bp, Homo sapiens
  source 1..1575: mol_type = genomic DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacaggtttgagctgaattatcacatgaatataaatgggaaatcagtgttttagagagagaacttttcgacatatttcctgttcccttggaataaaaacatttcttctgaaa

SEQ ID NO: 422 - DNA, 9676 bp, Homo sapiens
  source 1..9676: mol_type = genomic DNA
agttctccactggacttcagaacaagagggagctctgggctgctggctggttgtgcatttgctgtgggttccctccggcaggcgacctctccgcgctgagaaggttatccggataaccaatttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 423 - DNA, 5293 bp, Homo sapiens
  source 1..5293: mol_type = genomic DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 424 - DNA, 5417 bp, Homo sapiens
  source 1..5417: mol_type = genomic DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 425 - DNA, 5306 bp, Homo sapiens
  source 1..5306: mol_type = genomic DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 426 - DNA, 5430 bp, Homo sapiens
  source 1..5430: mol_type = genomic DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 427 - DNA, 2967 bp, Homo sapiens
  source 1..2967: mol_type = genomic DNA
gagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgcc

SEQ ID NO: 428 - DNA, 1211 bp, Homo sapiens
  source 1..1211: mol_type = genomic DNA
acattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacta

SEQ ID NO: 429 - protein, 8 aa, synthetic construct
GGGSGGGS

SEQ ID NO: 430 - protein, 4 aa, synthetic construct
GAGA

SEQ ID NO: 431 - protein, 4 aa, synthetic construct
GFLG

SEQ ID NO: 432 - RNA, 20 nt, synthetic construct
  source 1..20: mol_type = other RNA
gccgtacagttccacaaagg